(19) United States
(12) Patent Application Publication
(10) Pub. No.: US 2023/0277553 A1
(43) Pub. Date: Sep. 7, 2023

(54) THERAPEUTIC COMPOSITION OF CURE-PRO COMPOUNDS FOR TARGETED DEGRADATION OF BET DOMAIN PROTEINS, AND METHODS OF MAKING AND USAGE

(71) Applicant: CORNELL UNIVERSITY, Ithaca, NY (US)

(72) Inventors: Francis BARANY, New York, NY (US); Sarah F. GIARDANA, New York, NY (US); David J. WARREN, New York, NY (US)

(21) Appl. No.: 18/020,019

(22) PCT Filed: Aug. 4, 2021
(86) PCT No.: PCT/US2021/044432

§ 371 Date: Feb. 6, 2023

Related U.S. Application Data
(60) Provisional application No. 63/062,573, filed on Aug. 7, 2020.

(51) Int. Cl. A61K 31/5517

(52) U.S. Cl. CPC A61K 31/5517

(57) ABSTRACT

The present application is directed to a therapeutic composition, comprising two precursor compounds (monomers) that are suitable for assembly via two or more reversible covalent bonds. The monomers are polyfunctionalized molecules comprising a bioorthogonal linker element and ligand or pharmacophore, wherein the linker and ligand/pharmacophore are covalently coupled to each other either directly or through an optional connector moiety.

This application claims the priority benefit of U.S. Provisional Patent Application Ser. No. 63/062,573, filed Aug. 7, 2020, which is hereby incorporated by reference in its entirety

### FIELD

The present application is directed to therapeutically useful CURE-PRO compounds for targeted degradation of BET domain proteins, and methods of making and using them.

### BACKGROUND

Cancer is the leading cause of death in developed countries and the second leading cause of death in developing countries. Cancer has now become the biggest cause of mortality worldwide, with an estimated 9.6 million deaths from cancer in 2018. Cancer cases worldwide are forecast to rise by 75% and reach close to 25 million over the next two decades. Cancers arise due to mutations or dysregulation of genes involved in DNA replication and repair, cell cycle control, anchorage-independent growth, angiogenesis, apoptosis, tissue invasion, and metastasis (Hanahan et al., Cell 100(1):57-70 (2000)). These processes are controlled by networks of genes in the p53, cell cycle, apoptosis, Wnt signaling, RPTK signaling, and TGF-beta signaling pathways. Such genes and their protein products are the targets of many current and developing therapies.

Signaling pathways are used by cells to generate biological responses to external or internal stimuli. A few thousand gene products control both ontogeny/development of higher organisms and sophisticated behavior by their many different cell types. These gene products work in different combinations to achieve their goals via protein-protein interactions. The evolutionary architecture of such proteins is through modular protein domains that recognize and/or modify certain motifs. For example, different tyrosine kinases (such as Abl) will add phosphate groups to specific tyrosines imbedded in particular peptide sequences, while other enzymes (such as PTEN) act as phosphatases to remove certain signals. Proteins and other macromolecules may also be modified through methylation, acetylation, SUMOylation, neddylation, ubiquitination, and these signals in turn are recognized by specific domains that activate the next step in the pathway. Such pathways usually are initiated through signals to receptors on the surface, which move to intracellular protein interactions and often lead to signaling through transcription factor interactions that regulate gene transcription. For example, in the Wnt pathway, Wnt interacts with the Frizzled receptor, signaling through Disheveled, which inhibits the Axin-APC-GSK3 complex, which binds to beta-catenin to inhibit the combination of beta-catenin with TCF4, translocation of this complex into the nucleus, and activation of Myc, Cyclin D, and other oncogenic protein transcription (Polakis et al., Genes Dev. 14(15):1837-1851 (2000); Nelson et al., Science 303(5663):1483-1487 (2004)). Signaling may also proceed from the nucleus to secreted factors such as chemokines and cytokines (Charo et al., N. Engl. J. Med. 354(6):610-621 (2006)). Protein-protein and protein-nucleic acid recognition often work through protein interactions domains, such as the SH2, SH3, and PDZ domains. Currently, there are over 75 such motifs reported in the literature (Hunter et al., Cell 100:113-127 (2000); Pawson et al., Genes Dev. 14:1027-1047 (2000)). These protein-interaction domains comprise a rich opportunity for developing targeted therapies.

Traditional small molecule drugs are designed to inhibit enzyme active sites by fitting into deep pockets of proteins, which generally represents no more than 2-5% of the protein's surface area. These drugs have MW generally under 750 Daltons enabling diffusion across cellular membranes to reach their intracellular targets and are often orally bioavailable. However, because of their limited reach or “wingspan”, they are poorly suited to engage the shallower, more solvent-exposed, surfaces of proteins involved in protein-protein or protein-nucleic acid interactions. Thus, it is difficult to design small-molecule inhibitors targeted to these much more common regions of a protein found in transcription factors, scaffolding proteins, or proteins that lack a traditional enzymatic pocket. Further, even small molecules that bind to a protein-protein interaction surface may lack the ability to inhibit signaling or may be easily displaced by the protein-binding partner. In contrast, biologics, such as antibodies, do this quite well due to their large size. However, biologics cannot cross membranes, relegating them to solely extracellular targets. Thus, a fundamental conundrum is how to develop compounds capable of engaging relatively shallow surfaces of proteins via multi-point binding without becoming so large that cell permeability is compromised.

One approach to overcome some of these drug design limitations is the Coferon platform. Coferons are self-assembling molecules that are designed to come together upon binding to their target, where they form reversible covalent dimers through bio-orthogonal linker chemistries. These dimeric compounds demonstrate the enhanced binding affinities and selectivity of large molecules and exhibit superior cell permeability and properties of small molecules, for example, to achieve improved inhibition of Human beta-tryptase, BRD4, or c-MYC (U.S. Pat. Nos. 9,771,345; 8,853,185; and U.S. Pat. No. 9,943,603 to Barany et al.; Wanner et al., PloS one 10: e0121793 (2015); Giardina et al., ACS Med. Chem. Lett. 9(8): 827-831 (2018); Giardina et al., J. Med. Chem. 63(6):3004-3027 (2020)). Using the Coferon self-assembling drug molecule technology one can effectively deliver a bivalent molecule in two parts, cutting the molecular weight (MW) in half and permitting the flexibility to “tune” the structures for improved permeability, metabolic stability, bioavailability and pharmacokinetics, while retaining the superior affinity and specificity in the dimeric assembly. Even if the individual pharmacophores have average or poor binding affinities, the dimers may bind over a hundred-fold tighter than the monomers (Giardina et al., ACS Med. Chem. Lett. 9(8): 827-831 (2018); Giardina et al., J. Med. Chem. 63(6):3004-3027 (2020)). Several reversible linker chemistries have been developed and validated: Hindered diols and partner aryl boronic acids-based heterodimeric linkers (Wanner et al., PloS one 10: e0121793 (2015)); α-hydroxyketone-based homodimeric linkers (Giardina et al., ACS Med. Chem. Lett. 9(8): 827-831 (2018)); and benzoyl catechols, hydroxymethyl phenols, benzoyl methyl hydroxamates and partners benzoxaboroles or aryl boronic acids-based heterodimeric linkers (Giardina et al., J. Med. Chem. 63(6):3004-3027 (2020)).

An emerging theme for targeting “undruggable” proteins is to shift from an “occupancy” based strategy to an event-based strategy by targeting the protein for degradation using PROTACs (proteolysis-targeting chimeras) (Lu et al., Chem. Biol. 18; 22(6):755-63 (2015); Tanaka et al., Nat. Chem. Biol. 12(12):1089-1096 (2016); Lai and Crews, Nat Rev Drug Discov. 16(2):101-114 (2017); Bondeson and Crews, Annu. Rev. Pharmacol. Toxicol. 57:107-123 (2017); Salami and Crews, Science 355(6330):1163-1167 (2017)). PROTACs are bifunctional molecules that bind both a target protein and a member of an E3 ubiquitin ligase complex, bringing the two into proximity. The E3 ligase then mediates the transfer of ubiquitin from an E2 enzyme to the target protein, marking it for degradation by the proteasome (Sakamoto et al., Proc. Natl. Acad. Sci. USA 98: 8554-8559 (2001)). PROTACs have several advantages over conventional drugs. Whereas a classical drug must remain engaged with the target in order to inhibit its function, PROTACS can operate via a “hit and run” mechanism, where even a transient association of the bifunctional molecule with the target results in its ubiquitination and subsequent destruction. Thus, even if a target lacks a “molecular canyon” that can be targeted by classic small molecule with high affinity, one can make do with a lower affinity molecule that targets a surface feature of a protein in the context of a PROTAC (Zengerle et al., ACS Chem. Biol. 10:1770-1777 (2015); Lai et al., Angew. Chem. Int. Ed. 55:807-810 (2016); Gadd et al., Nat. Chem. Biol. 13:514-521 (2017)). Classical drug binding may stabilize proteins or lead to compensatory upregulation. In contrast, PROTACs have been shown to maintain protein knockdown (Lu et al., Chem. Biol. 22:755-763 (2015)), and PROTACs are therefore suitable for targeting proteins which accumulate or emerge as resistant upon inhibition. Further, PROTACs targeted against an oncogenic kinase (BTK) or a viral protein (HepC NS3/4a protease) suggest that they can overcome mutational escape (Buhimschi, et al.; Biochemistry. 3; 57(26):3564-3575 (2018); de Wispelaere, et al.; Nat. Commun. 10(1):3468 (2019)). However, considerable optimization is required to determine the ideal linker length for each target (Cyrus et al., Molecular bioSystems 7: 359-364 (2011); Cyrus et al., ChemMedChem 5:979-985 (2010)) in efforts to design PROTACs with good efficacy and bioavailability. The large size of these heterobifunctional compounds can produce poor drug-like properties, and with molecular weights typically in the 900-1000 Da range, the delivery and bioavailability of PROTAC drugs remain major challenges of this technology (Bondeson et al., Nat. Chem. Biol. 11:611-617 (2015); Neklesa et al., Pharmacol. Ther. 174:138-144 (2017)). One approach to try to overcome the high molecular weight and poor drug-like properties of PROTACs is to use “click chemistry” to irreversibly synthesize PROTACs within cells (Lebraud et al., ACS Cent. Sci. 2(12):927-934 (2016)). The authors used a tetrazine moiety appended to thalidomide and a trans-cyclo-octene moiety appended to the ligand of the target protein, which reacts in cells to form a cereblon E3 ligase recruiting “CLIPTAC” molecule. While an elegant demonstration for in vitro studies, this approach is not suitable for human use, since it requires providing the drug precursors to the patient sequentially, such that they do not form the product outside the target cells. Not only does this severely limit product yield, but products formed within the off-target cells cannot migrate into the target cells. Further, the irreversibly formed CLIPTAC creates high molecular weight compounds with the potential for causing liver damage. Two subsequent approaches assemble PROTAC molecules outside cells prior to testing them on cell lines, and thus teach away from the art of the current application. Using traditional azide and acetylene derivatives, click chemistry was used to assemble a BRD4 ligand (JQ1) to E3 ligase binders targeting cereblon (CRBN) and Von Hippel—Lindau (VHL) proteins to generate a family of PROTACs (Wurz et al., J. Med. Chem. 61(2):453-461 (2018)). In a two-stage strategy to identify optimal linker lengths, for the first stage, a few compounds comprising the estrogen receptor ligand connected to a hydrazide functional group were mixed with a few compounds comprising an E3 ligase ligand connected to a terminal aldehyde group. In the second stage, the acylhydrazone linkage of the best combination is replaced with a more stable amide linker to generate the full-length PROTAC (Roberts et al., ACS Chem. Biol. 15(6):1487-1496 (2020)). These approaches were specifically designed to assemble PROTACs as stable irreversible linkages prior to administering them—were they used in an attempt for in cell assembly, the azide moiety (Wurz et al.) or aldehyde moiety (Roberts et al.) appended to one of the ligands would react with off-target components in the cell, with the risk of significant toxicity or death.

Finally, it may be difficult to optimize the concentration of PROTACS for therapeutic use since too high a dose results in drug molecules fully binding the target, and fully binding the E3 ligase, but not simultaneously, while too low a dose results in binding either the target or E3 ligase, but again, not at the same time. This phenomenon is known as the “hook effect” and increases the risk for off-target degradation while trying to match the drug concentration to achieve optimal binding of both E3 ligase and the desired target (Bondenon et al., Cell Chem. Biol. 25(1):78-87 (2018)).

Thus, there is a need to design new small molecules that reversibly associate with good affinities for one another under physiological conditions to bring biological macromolecules into proximity with each other, enabling one or more subsequent macromolecule modification and/or degradation, and/or change in cellular transcription, epigenetic regulation, signal transduction, differentiation, apoptosis, or other cellular responses. The present application is directed at overcoming these and other deficiencies in the art.

### SUMMARY

One aspect of the present application is directed to a therapeutic composition comprising two precursor compounds (monomers) that are suitable for assembly via two or more reversible covalent bonds of the linker elements of each monomer. The monomers are polyfunctionalized molecules comprising a bioorthogonal linker element and ligand or pharmacophore, wherein the linker and ligand/pharmacophore are covalently coupled to each other either directly or through an optional connector moiety.

A first aspect of the present application relates to a therapeutic composition comprising:
a first precursor compound having the chemical structure:

E3ULB—C₁-L₁,

or a pharmaceutically acceptable salt, enantiomer, stereoisomer, solvate, or polymorph thereof, and
a second precursor compound having the chemical structure:

TPB—C₂-L₂,

or a pharmaceutically acceptable salt, enantiomer, stereoisomer, solvate, or polymorph thereof, wherein:
E3ULB is a small molecule E3 ubiquitin ligase binding moiety that binds an E3 ubiquitin ligase, an E3 ubiquitin ligase complex, or subunit thereof,
TPB is a small molecule comprising a BET domain protein binding moiety,
C₁ and C₂ are independently a bond or a connector element,
L₁ and L₂ are linker element pairs suitable for binding to one another by two or more reversible covalent bonds that form under physiological conditions, each linker element having a molecular weight of 54 to 420 Daltons, said linker element pairs being selected from the group consisting of.
(1) one linker element comprising an aromatic 1,2-diol-containing moiety and the other linker element comprising an aromatic or heteroaromatic boronic acid- or boronic ester-containing moiety;
(2) one linker element comprising an aromatic 1,2-carbonyl and alcohol-containing moiety and the other linker element comprising an aromatic or heteroaromatic boronic acid- or boronic ester-containing moiety;
(3) one linker element comprising a cis-dihydroxycoumarin-containing moiety and the other linker element comprising an aromatic or heteroaromatic boronic acid- or boronic ester-containing moiety;
(4) one linker element comprising an α-hydroxycarboxylic acid-containing moiety and the other linker element comprising an aromatic or heteroaromatic boronic acid- or boronic ester-containing moiety;
(5) one linker element comprising an aromatic 1,3-diol-containing moiety and the other linker element comprising an aromatic or heteroaromatic boronic acid- or boronic ester-containing moiety;
(6) one linker element comprising an aromatic 2-(aminomethyl)phenol-containing moiety and the other linker element comprising an aromatic or heteroaromatic boronic acid- or boronic ester- or 1,2-boronic acid and carbonyl-containing moiety;
(7) one linker element comprising a cis-1,2-diol-, or cis-1,3-diol-, or a ring system comprising a trans-1,2-diol-containing moiety and the other linker element comprising an aromatic or heteroaromatic boronic acid- or boronic ester-containing moiety;
(8) one linker element comprising a [2.2.1] bicyclic ring system comprising a cis-1,2-diol-, or a cis-1,2-diol and cis-1,3-diol-, or a cis-1,2-diol and a β-hydroxyketone-containing moiety and the other linker element comprising an aromatic or heteroaromatic boronic acid- or boronic ester-containing moiety;
(9) one linker element comprising a [2.2.1] bicyclic ring system comprising a cis-1,2-diol and cis-1,2-aminoalcohol-, or a cis-1,2-diol and cis-1,3-aminoalcohol-, or a cis-1,2-diol and cis-1,2-hydrazine-alcohol-containing moiety and the other linker element comprising an aromatic or heteroaromatic boronic acid- or 1,2-boronic acid and carbonyl-containing moiety;
(10) one linker element comprising a [2.2.1] bicyclic ring system comprising a cis-1,2-aminoalcohol and cis-1,3-diol- or a cis-1,2-aminoalcohol and a β-hydroxyketone-containing moiety and the other linker element comprising an aromatic or heteroaromatic boronic acid- or 1,2-boronic acid and carbonyl-containing moiety;
(11) one linker element comprising a cis-1,2-aminoalcohol-, or a ring system comprising a trans-1,2-aminoalcohol-containing an aromatic or heteroaromatic boronic acid- or boronic ester- or 1,2-boronic acid and carbonyl-containing moiety;
(12) one linker element comprising a cis-1,3-aminoalcohol-containing moiety and the other linker element comprising an aromatic or heteroaromatic boronic acid- or boronic ester- or 1,2-boronic acid and carbonyl-containing moiety;
(13) one linker element comprising an acyl or aromatic hydrazine-containing moiety and the other linker molecule comprising an aromatic or heteroaromatic 1,2-boronic acid and carbonyl-containing moiety; and
(14) one linker element comprising an α-hydroxyketone-containing moiety and the other linker molecule comprising an α-hydroxyketone-containing moiety.

A second aspect of the present application relates to a method of binding to and redirecting the specificity of an E3 ubiquitin ligase, an E3 ubiquitin ligase complex, or subunit thereof to induce the ubiquitination and degradation of a BET domain protein in a biological sample. The method includes contacting the sample with the therapeutic composition of the present application.

A further aspect of the present application relates to a method of treating a BET domain protein mediated disorder, condition, or disease in a patient. The method includes administering to the patient the therapeutic composition of the present application.

A final aspect of the present application relates to a method of treatment including selecting a subject with a BET domain protein mediated disorder, condition, or disease; and administering to the selected subject the therapeutic composition of the present application.

CURE-PROs (Combinatorial Ubiquitination REal-time PROteolysis) are orally active drugs that can enter cells and, once inside, reversibly combine with each other under physiological conditions to bring biological macromolecules into proximity with each other, preferably resulting in the degradation of one of these macromolecules. CURE-PROs have repurposed the reversible linkers from the Coferon platform to generate reversible hetero-bifunctional PROTAC compounds from two smaller precursors. The modular design of CURE-PROS allows for the rapid and cost-effective optimization of the connector length and is readily amenable to screening for new targets.

A CURE-PRO monomer is composed of a pharmacophore or ligand and a linker element (FIG. 1A). The linker element has a molecular weight in the range of about 54-420 Daltons; it is responsible for covalently combining with its partner linker element under physiological conditions using reversible chemistry. The linker element can have a dissociation constant up to 1 M, preferably in the range of 100 nM to 100 μM. A pharmacophore or ligand is provided to bind to a target protein, such as a BET domain target protein (i.e. BRD4), and generally has a molecular weight in the range of about 150 to 800 Daltons with a dissociation constant of less than 300 μM, preferably in the range of 1 nM to 100 μM. A ligand is provided to bind to an E3 ligase or ligase machinery (E3ULB) and generally has a molecular weight in the range of about 150 to 800 Daltons with a dissociation constant of less than 300 μM, preferably in the range of 1 nM to 100 μM. The linker element and the pharmacophore may be directly attached to each other or linked together by a connector moiety. The pharmacophore (or ligand) may comprise of a portion of the linker or connector, and the linker or connector may comprise of a portion of the pharmacophore (or ligand). Thus, a given monomer always comprises of a pharmacophore (or ligand) moiety and a linker element, but certain moieties or structures within the monomer may play dual roles as both pharmacophore (or ligand) moiety and linker element, which are coupled through one or more chemical bonds or connectors

### BRIEF DESCRIPTION OF THE DRAWINGS

FIGS. 1A-1B are schematic drawing of the CURE-PRO drug platform. FIG. 1A is a schematic drawing of the components used in CURE-PRO monomers. FIG. 1B is a schematic drawing of CURE-PRO monomers in equilibrium with CURE-PRO dimers in equilibrium with the CURE-PRO components binding to both the protein target and E3 ligase, bringing them in proximity to enable polyubiquitination of the protein target via the E2 ubiquitin-conjugating enzyme, thus marking the protein target for degradation by the 26S Proteasome.

FIG. 2 shows variations of CURE-PRO heterodimers are designed to exploit different ubiquitin-proteasome degradation pathways. Part A is a schematic drawing of a CURE-PRO heterodimer recruiting the MDM2 E3 ligase to the protein target, enabling polyubiquitination via E2, and subsequent degradation via the 26S proteasome. Part B is a schematic drawing of a CURE-PRO heterodimer recruiting the CULLIN2-Elongin B-Elongin C-VHL complex to the protein target. Part C is a schematic drawing of a CURE-PRO heterodimer recruiting the CULLIN4-DDB1-CRBN complex to the protein target. After protein degradation the CURE-PRO monomers are liberated and available for catalytic degradation of another molecule of the protein target.

FIG. 3 is a schematic drawing of an AlphaScreen assay to identify potential pharmacophores that preferentially recruit an E3 ligase or adaptor protein to the target protein.

FIG. 4 is a schematic drawing of an in cellular screen for native protein target degradation in the presence of a CURE PRO molecule comprising a pharmacophore for the native protein target and a CURE PRO molecule comprising a ligand to an endogenous E3 ligase (machinery). Degradation of the native protein target results in a phenotypic change (illustrated as a change in cell shape in the bottom diagram) that is scored by a fluorescent, colorimetric, or luminescent assay.

FIG. 5 is a schematic drawing of an in cellular screen for native protein target degradation in the presence of a CURE PRO molecule comprising a pharmacophore for the native protein target and a CURE PRO molecule comprising a ligand to an endogenous E3 ligase (machinery). A host protein is genetically or chemically modified with a first reporter group (R-1), and the target protein is modified with a second reporter group (R-2). Degradation of the native protein target results in loss of R-2 but not R-1 reporter signal that is scored by a fluorescent, colorimetric, or luminescent assay.

FIGS. 6A-6B depict the CURE-PRO-mediated BRD4 degradation, as detected by Western blot (FIG. 6A), and the structures (FIG. 6B) of the reversibly binding ligands, with the BRD ligand (BRD-N69, top) and cereblon binding ligands (8048, bottom left; 8049, bottom right). Degradation is noted with BRD-N69 and 8048, but not 8049.

FIGS. 7A-7B depict the CURE-PRO-mediated BRD4 degradation, as detected by Western blot (FIG. 7A), and the structures (FIG. 7B) of the reversibly binding ligands, with the BRD ligand (BRD-N70, top) and cereblon binding ligands (8048, bottom left; 8049, bottom right). Degradation is noted with BRD-N70 and 8048, but not 8049.

FIGS. 8A-B depict the CURE-PRO-mediated BRD4 degradation for 8048, 8049 and BRD-N71 monomers in combination in a 1:1 ratio as detected by Western blot (FIG. 8A). The structures (FIG. 8B) of the reversibly binding ligands, with the BRD ligand (BRD-N71, top) and cereblon binding ligands (8048, bottom left, and 8049, bottom right) are shown. Some selectivity for BRD-N71 and 8048 is noted.

FIGS. 9A-9C depict the CURE-PRO-mediated BRD4 degradation (FIG. 9A: BRD-N30; FIG. 9B: BRD-N38), as detected by Western blot, and the structures (FIG. 9C) of the reversibly binding ligands, with the BRD ligands (BRD-N30, BRD-N38; left) and cereblon binding ligand (8048, right).

FIGS. 10A-10C depict the CURE-PRO-mediated BRD4 degradation (FIG. 10A: BRD-N44; FIG. 10B: BRD-N67), as detected by Western blot, and the structures (FIG. 10C) of the reversibly binding ligands, with the BRD ligands (BRD-N44, BRD-N67; left) and cereblon binding ligand (8048, right).

FIGS. 11A-11C depict the CURE-PRO-mediated BRD4 degradation (FIG. 11A: BRD-N39; FIG. 11B: BRD-N67), as detected by Western blot, and the structures (FIG. 11C) of the reversibly binding ligands, with the BRD ligands (BRD-N39, BRD-N67: left) and cereblon binding ligands (8048, 8049: right).

FIGS. 12A-12B depict the CURE-PRO-mediated BRD4 degradation, as detected by Western blot (FIG. 12A), and the structures (FIG. 12B) of the reversibly binding ligands, with the BRD ligand (BRD-N1, top) and cereblon binding ligands (8048, bottom left; 8049, bottom right). Degradation is noted with BRD-N1 and 8048, but not 8049.

FIGS. 13A-13B depict the CURE-PRO-mediated BRD4 degradation, as detected by Western blot (FIG. 13A), and the structures (FIG. 13B) of the reversibly binding ligands, with the BRD ligand (BRD-N5, top) and cereblon binding ligands (8048, bottom left; 8049, bottom right). Degradation is noted with BRD-N5 and 8048, but not 8049.

FIGS. 14A-14B depict the CURE-PRO-mediated BRD4 degradation, as detected by Western blot (FIG. 14A), and the structures (FIG. 14B) of the reversibly binding ligands, with the BRD ligand (BRD-N6, top) and cereblon binding ligands (8048, bottom left, 8049 bottom right). Degradation is noted with BRD-N6 and 8048, but not 8049.

FIGS. 15A-15B depict the CURE-PRO-mediated BRD4 degradation, as detected by Western blot (FIG. 15A), and the structures (FIG. 15B) of the reversibly binding ligands, with the BRD ligand (BRD-N22, top) and cereblon binding ligands (8048, bottom left; 8049, bottom right). Degradation is noted with BRD-N22 and 8048, but not 8049.

FIGS. 16A-16B depict the CURE-PRO-mediated BRD4 degradation, as detected by Western blot (FIG. 16A), and the structures (FIG. 16B) of the reversibly binding ligands, with the BRD ligand (BRD-N39, top) and cereblon binding ligands (8048, bottom left; 8049, bottom right). Degradation is noted with BRD-N39 and 8048, but not 8049.

FIGS. 17A-17B depict the concentration-dependence of CURE-PRO-mediated BRD4 degradation, as detected by Western blot (FIG. 17A), and the structures (FIG. 17B) of the reversibly binding ligands, with the BRD ligand (BRD-N67, left) and cereblon binding ligand (8048, right) and the heterodimer are shown.

FIGS. 18A-18B depict the concentration-dependence of CURE-PRO-mediated BRD4, as detected by Western Blot (FIG. 18A), degradation and the structures (FIG. 18B) of the reversibly binding ligands, with the BRD ligand (BRD-N10, top) and cereblon binding ligands (8048, bottom left, and 8049, bottom right). Both 8048 and 8049 at high concentrations with BRD-N10 are capable of degrading BRD4.

FIGS. 19A-19B depict dose-response curves for CURE-PRO-mediated toxicity in MV-411 cells as measured using Cell-titer Glo (Promega) (FIG. 19A). BRD-N2 (left) increased toxicity when cotreated with cereblon ligand 8049 (right), at 1:1 ratios RLU, relative luminescence units. The monomers and self-assembled dimer are shown in FIG. 19B.

FIGS. 20A-20B depict dose-response curves for CURE-PRO-mediated toxicity in MV-411 cells as measured using Cell-titer Glo (Promega) (FIG. 20A). BRD-N8 (left) increased toxicity when cotreated with cereblon ligand 8049 (right), at 1:1 ratios RLU, relative luminescence units. The monomers and self-assembled dimer are shown in FIG. 20B.

FIGS. 21A-B depict dose-response curves for CURE-PRO-mediated toxicity in MV-411 cells as measured using Cell-titer Glo (Promega) (FIG. 21A). BRD-N10 (left) increased toxicity when cotreated with cereblon ligand 8049 (right), at 1:1 ratios RLU, relative luminescence units. The monomers and self-assembled dimer are shown in FIG. 21B.

FIGS. 22A-22B depict dose-response curves for CURE-PRO-mediated toxicity in MV-411 cells as measured using Cell-titer Glo (Promega) (FIG. 22A). BRD-N25 (left) increased toxicity when cotreated with cereblon ligand 8049 (right), at 1:1 ratios RLU, relative luminescence units. The monomers and self-assembled dimer are shown in FIG. 22B.

FIGS. 23A-23B depicts the CURE-PRO-mediated BRD4 degradation, as detected by Western blot (FIG. 23A), and the structures (FIG. 23B) of the reversibly binding ligands, with the BRD ligand (BRD-E8, top) and cereblon binding ligands (8046, bottom left; 8047, bottom middle; 8066, bottom right). Degradation is noted with BRD-E8 and 8046 and 8066, but not 8047.

FIGS. 24A-24B depict the CURE-PRO-mediated BRD4 degradation, as detected by Western blot (FIG. 24A), and the structures (FIG. 24B) of the reversibly binding ligands, with the BRD ligand (BRD-E14, top) and cereblon binding ligands (8046, bottom left; 8047, bottom middle; 8066, bottom right). Degradation is noted with BRD-E14 and 8047, but not 8046 nor 8066.

FIGS. 25A-25B depict the CURE-PRO-mediated BRD4 degradation, as detected by Western blot (FIG. 25A), and the structures (FIG. 25B) of the reversibly binding ligands, with the BRD ligand (BRD-E20, top) and cereblon binding ligands (8046, bottom left; 8047, bottom middle; 8066, bottom right). Degradation is noted with BRD-E20 and 8046 and 8066, but not 8047.

FIGS. 26A-26B depict the CURE-PRO-mediated BRD4 degradation, as detected by Western blot (FIG. 26A), and the structures (FIG. 26B) of the reversibly binding ligands, with the BRD ligand (BRD-E29, top) and cereblon binding ligands (8046, bottom left; 8047, bottom middle; 8066, bottom right). Degradation is noted with BRD-E29 and all cereblon ligands.

FIGS. 27A-27B depict the CURE-PRO-mediated BRD4 degradation, as detected by Western blot (FIG. 27A), and the structures (FIG. 27B) of the reversibly binding ligands, with the BRD ligand (BRD-E4, top) and cereblon binding ligands (8046, bottom left; 8047, bottom middle; 8066, bottom right). Degradation is noted with BRD-E4 in combination at a 1:1 ratio with all the cereblon ligands.

FIGS. 28A-28B depict the CURE-PRO-mediated BRD4 degradation, as detected by Western blot (FIG. 28A), and the structures (FIG. 28B) of the reversibly binding ligands, with the BRD ligand (BRD-E46, top) and cereblon binding ligands (8046, bottom left; 8066, bottom right). Degradation is noted with BRD-E46 in a 1:1 ratio with 8066 and 8046.

FIGS. 29A-29B depict the CURE-PRO-mediated BRD4 degradation, as detected by Western blot (FIG. 29A), and the structures (FIG. 29B) of the reversibly binding ligands, with the BRD ligand (BRD-E43, top) and cereblon binding ligands (8046, bottom left; 8066, bottom right). Degradation is noted with BRD-E43 and 8066, but not 8046.

FIGS. 30A-30B depict the CURE-PRO-mediated BRD4 degradation, as detected by Western blot (FIG. 30A), and the structures (FIG. 30B) of the reversibly binding ligands, with the BRD ligand (BRD-E79, top) and cereblon binding ligands (8046, bottom left; 8066, bottom right). Degradation is noted with BRD-E79 and 8066 and 8046.

FIGS. 31A-31B depict the CURE-PRO-mediated BRD4 degradation, as detected by Western blot (FIG. 31A), and the structures (FIG. 31B) of the reversibly binding ligands, with the BRD ligand (BRD-E5, top) and cereblon binding ligands (8046, bottom left; 8047, bottom middle, 8066, bottom right). Degradation is noted with BRD-E5 and 8047 and 8066 cereblon ligands.

FIGS. 32A-32C depict the CURE-PRO-mediated BRD4 degradation (FIG. 32A: BRD-E42; FIG. 32B: BRD-E43), as detected by Western blot, and the structures (FIG. 32C) of the reversibly binding ligands, with the BRD ligands (BRD-E42, BRD-E43; left) and cereblon binding ligand (8047, right).

FIGS. 33A-33C depict the CURE-PRO-mediated BRD4 degradation (FIG. 33A: BRD-E52; FIG. 33B: BRD-E27), as detected by Western blot, and the structures (FIG. 33C) of the reversibly binding ligands, with the BRD ligands (BRD-E52, BRD-E27; left) and cereblon binding ligand (8047, right).

FIGS. 34A-34C depict the CURE-PRO-mediated BRD4 degradation (FIG. 34A: BRD-E76; FIG. 34B: BRD-E8), as detected by Western blot, and the structures (FIG. 34C) of the reversibly binding ligands, with the BRD ligands (BRD-E76, BRD-E8; left) and cereblon binding ligands (8046, 8066; right).

FIGS. 35A-35C depict the CURE-PRO-mediated BRD4 degradation, as detected by Western blot, (FIG. 35A: BRD-E45; FIG. 35B: BRD-E74), and the structures (FIG. 35C) of the reversibly binding ligands, with the BRD ligands (BRD-E45, BRD-E74; left) and cereblon binding ligands (8066, 8046; right).

FIGS. 36A-36C depict the CURE-PRO-mediated BRD4 degradation (FIG. 36A: BRD-E40; FIG. 36B: BRD-E41), as detected by Western blot, and the structures (FIG. 36C) of the reversibly binding ligands, with the BRD ligands (BRD-E40, BRD-E41; left) and cereblon binding ligand (8066, right).

FIGS. 37A-37B depict the CURE-PRO-mediated BRD4 degradation for the BRD-E4 monomer and for BRD-E4 and 8046 combined in a 1:1 ratio, as detected by Western blot (FIG. 37A). The (FIG. 37B) structures of the reversibly binding ligands, with the BRD ligand (BRD-E4, left) and cereblon binding ligand (8046, right) and the heterodimer are shown.

FIGS. 38A-38B depict the CURE-PRO-mediated BRD4 degradation, as detected by Western blot, and the structures of the reversibly binding ligands, with the BRD ligand (BRD-E10, top and cereblon binding ligands (8046, bottom left; 8047, bottom middle; 8066, bottom right).

FIGS. 39A-39B depict the concentration-dependence of CURE-PRO-mediated BRD4 degradation, as detected by Western blot (FIG. 39A), and the structures (FIG. 39B) of the reversibly binding ligands, with the BRD ligand (BRD-E8, left) and cereblon binding ligand (8046, right).

FIGS. 40A-40B depict the concentration-dependence of CURE-PRO-mediated BRD4 degradation, as detected by Western blot (FIG. 40A), and the structures (FIG. 40B) of the reversibly binding ligands, with the BRD ligand (BRD-E21, left), the cereblon binding ligand (8047, right) and the reversible heterodimer (bottom).

FIGS. 41A-41B depict the concentration-dependence of CURE-PRO-mediated BRD4 degradation, as detected by Western Blot (FIG. 41A), and the structures (FIG. 41B) of the reversibly binding ligands, with the BRD ligand (BRD-E30, left), the cereblon binding ligand (8047, right), and the reversible heterodimer (bottom).

FIGS. 42A-42B depict the concentration-dependence of CURE-PRO-mediated BRD4 degradation, as detected by Western Blot (FIG. 42A), and the structures (FIG. 42B) of the reversibly binding ligands, with the BRD ligand (BRD-E72, left), the cereblon binding ligand (8047, right), and the reversible heterodimer (bottom).

FIGS. 43A-43B depict the concentration-dependence of CURE-PRO-mediated BRD4, as detected by Western Blot (FIG. 43A), degradation and the structures (FIG. 43B) of the reversibly binding ligands, with the BRD ligand (BRD-E79, left), the cereblon binding ligand (8047, right), and the reversible heterodimer (bottom).

FIGS. 44A-44B depict the CURE-PRO-mediated BRD4 degradation, as detected on a WES capillary electrophoresis instrument (Proteinsimple) (FIG. 44A), and the structures (FIG. 44B) of the reversibly binding ligands, with the BRD4 ligand (BRD-E52, top) and CRBN binding ligands (8046, bottom left, 8047, bottom middle, and 8066, bottom right). Co-dosing with CRBN ligand 8047 demonstrates marked BRD4 degradation after 4h with sustained degradation for up to 8h after drugs are washed out.

FIGS. 45A-45B depict the CURE-PRO-mediated BRD4 degradation, as detected on a WES capillary electrophoresis instrument (Proteinsimple) (FIG. 45A), and the structures (FIG. 45B) of the reversibly binding ligands, with the BRD4 ligand (BRD-E72, top) and CRBN binding ligands (8046, bottom left, 8047, bottom middle, and 8066, bottom right). Co-dosing with CRBN ligand 8047 demonstrates marked BRD4 degradation after 4h with sustained degradation for up to 8h after drugs are washed out.

FIGS. 46A-46B depict the concentration-dependent CURE-PRO-mediated BRD4 degradation, as detected on a WES capillary electrophoresis instrument (Proteinsimple) (FIG. 46A), and the structures (FIG. 46B) of the reversibly binding ligands, with the BRD4 ligand (BRD-E52, top), the non-dimerizable control (BRD-E52C) and CRBN binding ligand (8047, bottom). Co-dosing with CRBN ligand 8047 demonstrates marked BRD4 degradation in the presence of the monomer capable of forming a dimer (BRD-E52), but not BRD-E52C. Monomers alone did not alter BRD4 expression.

FIGS. 47A-47B depict dose-response curves for CURE-PRO-mediated toxicity in MV-411 cells as measured using Cell-titer Glo (Promega) (FIG. 47A) and the relevant structures (FIG. 47B). BRD-E20 (top) increased toxicity when cotreated with cereblon ligands 8066 (bottom right), 8046 (bottom left), and 8047 (bottom middle), when compared to treatment with monomers alone. RLU, relative luminescence units.

FIGS. 48A-48B depict dose-response curves for CURE-PRO-mediated toxicity in MV-411 cells as measured using Cell-titer Glo (Promega) (FIG. 48A) and the relevant structures (FIG. 48B). BRD-E29 (top) increased toxicity when cotreated with cereblon ligands 8066 (bottom right), 8046 (bottom left), and 8047 (bottom middle), when compared to treatment with monomers alone. RLU, relative luminescence units.

FIGS. 49A-49B depict dose-response curves for CURE-PRO-mediated toxicity in MV-411 cells as measured using Cell-titer Glo (Promega) (FIG. 49A) and the relevant structures (FIG. 49B). BRD-E41 (top) increased toxicity when cotreated with cereblon ligands 8066 (bottom right), 8046 (bottom left), and 8047 (bottom middle), when compared to treatment with monomers alone. RLU, relative luminescence units.

FIGS. 50A-50B depict dose-response curves for CURE-PRO-mediated toxicity in MV-411 cells as measured using Cell-titer Glo (Promega) (FIG. 50A) and the relevant structures (FIG. 50B). BRD-E46 (top) increased toxicity when cotreated with cereblon ligands 8066 (bottom right), 8046 (bottom left), and 8047 (bottom middle), when compared to treatment with monomers alone. RLU, relative luminescence units.

FIGS. 51A-51B depict dose-response curves for CURE-PRO-mediated toxicity in MV-411 cells as measured using Cell-titer Glo (Promega) (FIG. 51A) and the relevant structures (FIG. 51B). BRD-E73 (top) increased toxicity when cotreated with cereblon ligands 8066 (bottom right), 8046 (bottom left), and 8047 (bottom middle), when compared to treatment with monomers alone. RLU, relative luminescence units.

FIGS. 52A-52B depict dose-response curves for CURE-PRO-mediated toxicity in MV-411 cells as measured using Cell-titer Glo (Promega) (FIG. 52A) and the relevant structures (FIG. 52B). BRD-E75 (top) increased toxicity when cotreated with cereblon ligands 8066 (bottom right), 8046 (bottom left), and 8047 (bottom middle), when compared to treatment with monomers alone. RLU, relative luminescence units.

FIGS. 53A-53B depict dose-response curves for CURE-PRO-mediated toxicity in MV-411 cells as measured using Cell-titer Glo (Promega) (FIG. 53A) and the relevant structures (FIG. 53B). BRD-E51 (top) increased toxicity when cotreated with cereblon ligands 8046 (bottom left), 8066 (bottom right), and 8047 (bottom middle), when compared to treatment with monomers alone. RLU, relative luminescence units.

FIGS. 54A-54B depict dose-response curves for CURE-PRO-mediated toxicity in MV-411 cells as measured using Cell-titer Glo (Promega) (FIG. 54A) and the relevant structures (FIG. 54B). BRD-E76 (top) increased toxicity when cotreated with cereblon ligands 8046 (bottom left), 8066 (bottom right), and 8047 (bottom middle), when compared to treatment with monomers alone. RLU, relative luminescence units.

FIGS. 55A-55B depict dose-response curves for CURE-PRO-mediated toxicity in MV-411 cells as measured using Cell-titer Glo (Promega) (FIG. 55A) and the relevant structures (FIG. 55B). BRD-E78 (top) increased toxicity when cotreated with cereblon ligands 8046 (bottom left), 8066 (bottom right), and 8047 (bottom middle), when compared to treatment with monomers alone. RLU, relative luminescence units.

FIGS. 56A-56B depict the concentration-dependence loss of cell viability as determined by CellTiter-Glo® Luminescent Cell Viability Assay (Promega) (FIG. 56A), the structures (FIG. 56B) of the reversibly binding ligands, with the BRD4 ligand (BRD-E72, left) and CRBN binding ligand (8047, right middle). Co-dosing BRD-E72 with the CRBN ligand, 8047, demonstrates marked loss in cell viability when compared to monomer treatment alone.

FIGS. 57A-57B depict the CURE-PRO-mediated caspase activation, as detected by Caspase-Glo® 3/7 Assay System (Promega) (FIG. 57A), and the structures (FIG. 57B) of the reversibly binding ligands, with the BRD4 ligands (BRD-E52 top left, and BRD-E72, top right) and CRBN binding ligand (8047, bottom). Co-dosing BRD ligands with CRBN ligand 8047 demonstrates marked caspase activation, but not with monomers alone.

FIGS. 58A-58C depict the inhibition of CURE-PRO-mediated BRD4 degradation, as detected by Western Blot, by the preincubation of pomalidomide at equimolar final concentrations with FIG. 58A: BRD-E52 or FIG. 58B: BRD-E72. The structures (FIG. 58C) of the reversibly binding ligands are shown with the BRD4 ligand (BRD-E52, BRD-E72; left) and cereblon binding ligands (8047, right).

FIGS. 59A-59B depict the activity of CURE-PRO-mediated degradation, as detected on a WES capillary electrophoresis instrument (Proteinsimple) (FIG. 59A), against BRD4. The structures (FIG. 59B) of the reversibly binding ligands are shown with the BRD ligand (BRD-E8, top) and MDM2 binding ligands (8314, bottom left, and 8313, bottom right). Degradation is observed when BRD-E8 is co-dosed with 8314, but not and 8313.

FIGS. 60A-60B depicts the activity of CURE-PRO-mediated degradation, as detected on a WES capillary electrophoresis instrument (Proteinsimple) (FIG. 60A), against BRD4. The structures (FIG. 60B) of the reversibly binding ligands are shown with the BRD4 ligands (BRD-E14, top left; BRD-E20, top middle; BRD-E21, top right) and MDM2 binding ligands (8314, bottom left, and 8313, bottom right). Degradation is observed when BRD-E14, BRD-E20 and BRD-E21 are co-dosed with 8314, but not and 8313.

FIGS. 61A-61B depict the activity of CURE-PRO-mediated degradation, as detected on a WES capillary electrophoresis instrument (Proteinsimple) (FIG. 61A), against BRD4. The structures (FIG. 61B) of the reversibly binding ligands are shown with the BRD ligand (BRD-E79, top) and MDM2 binding ligands (8314, bottom left, and 8313, bottom right). Degradation of BRD4 is observed when BRD-E79 is co-dosed with 8313, but not and 8314.

FIGS. 62A-62B depict the CURE-PRO-mediated BRD4 degradation, as detected on a WES capillary electrophoresis instrument (Proteinsimple) (FIG. 62A), and the structures (FIG. 62B) of the reversibly binding ligands, with the BRD4 ligand (BRD-N25, top) and MDM2 binding ligands (8310, bottom left, and 8312, bottom right). Ligands 8310 and 8312 caused degradation when co-dosed with BRD-N25.

FIGS. 63A-63B depict the CURE-PRO-mediated BRD4 degradation, as detected on a WES capillary electrophoresis instrument (Proteinsimple) (FIG. 63A), and the structures (FIG. 63B) of the reversibly binding ligands, with the BRD4 ligand (BRD-N39, top) and MDM2 binding ligands (8310, bottom left, and 8312, bottom right). Ligands 8310 and 8312 caused degradation when co-dosed with BRD-N39.

FIGS. 64A-64B depict the activity of CURE-PRO-mediated degradation, as detected by Western Blot (FIG. 64A), against the BRD protein family. The structures (FIG. 64B) of the reversibly binding ligands are shown with the BRD ligand (BRD-N25, top) and MDM2 binding ligands (8310, bottom left, and 8312, bottom right). Degradation is evident for BRD2, BRD3 and BRD4, while some selectivity for BRD3 and BRD4 over BRD2 for 8310 is noted.

FIGS. 65A-65B depict the activity of CURE-PRO-mediated degradation, as detected by Western Blot (FIG. 65A), against the BRD protein family. The structures (FIG. 65B) of the reversibly binding ligands are shown with the BRD ligand (BRD-N39, top) and MDM2 binding ligands (8310, bottom left, and 8312, bottom right). Degradation is evident for BRD2, BRD3 and BRD4, while some selectivity for BRD4 over BRD2 and BRD3 is noted.

FIGS. 66A-66B depict the activity of CURE-PRO-mediated suppression of the downstream target gene of c-MYC, SLC19A1, after BRD4 degradation (FIG. 66A). The structures (FIG. 66B) of the reversibly binding ligands are shown with the BRD4 ligand (BRD-N25, top) and MDM2 binding ligands (8310, bottom left, and 8312, bottom right). SLC19A1 is suppressed more substantially with BRD-N25 and 8312, than BRD-N25 and 8310, JQ1 pomalidomide (pom) or the ligands alone. ARV-825 completely suppressed SLC19A1 expression. UD, undetermined. ACTINB and GAPDH indicated equal RNA loading. Ct values are shown.

FIGS. 67A-67B depict the activity of CURE-PRO-mediated suppression of the downstream target gene of c-MYC, SLC19A1, after BRD4 degradation (FIG. 67A). The structures (FIG. 67B) of the reversibly binding ligands are shown with the BRD4 ligand (BRD-N39, top) and MDM2 binding ligands (8310, bottom left, and 8312, bottom right). SLC19A1, is completely suppressed with BRD-N39 and 8312, whereas BRD-N39 and 8310 show suppression to levels comparable to that of JQ1 treatment. Pomalidomide (pom) or the ligands alone show no suppression of SLC19A1 expression. ARV-825 completely suppressed SLC19A1 expression. UD, undetermined. ACTINB and GAPDH indicated equal RNA loading. Ct values are shown.

FIGS. 68A-68B depict the dependence of the proteasome for CURE-PRO-mediated BRD4 degradation, as detected on a WES capillary electrophoresis instrument (Proteinsimple) (FIG. 68A), and the structures (FIG. 68B) of the reversibly binding ligands, with the BRD ligand (BRD-N25, left), and MDM2 binding ligand (8310, right). Co-dosing with MDM2 ligand 8310 demonstrates marked BRD4 degradation that is inhibited with the proteasome inhibitors, MG-132 and Carfilzomib.

FIGS. 69A-69B depict the CURE-PRO-mediated BRD4 degradation, as determined by Western Blot (FIG. 69A) and the structures (FIG. 69B) of the reversibly binding ligands, with the BRD ligand (BRD-E9, left), the VHL binding ligand (8305, right, and the reversible heterodimer (bottom).

FIGS. 70A-70B depict the CURE-PRO-mediated BRD4 degradation, as detected by Western Blot (FIG. 70A), and the structures (FIG. 70B) of the reversibly binding ligands, with the BRD ligand (BRD-E20, left), the VHL binding ligand (8305, right), and the reversible heterodimer (bottom).

FIGS. 71A-71B depict the CURE-PRO-mediated BRD4 degradation, as determined by Western Blot (FIG. 71A), and the structures (FIG. 71B) of the reversibly binding ligands, with the BRD4 ligand (BRD-E50, top) and VHL binding ligands (8304, bottom left, and 8305, bottom right). Co-dosing with VHL ligand 8305 demonstrates marked BRD4 degradation, whereas no degradation is noted with 8304.

FIGS. 72A-72B depict the CURE-PRO-mediated BRD4 degradation, as detected on a WES capillary electrophoresis instrument (Proteinsimple) (FIG. 72A), and the structures (FIG. 72B) of the reversibly binding ligands, with the BRD4 ligand (BRD-E50, top) and VHL binding ligands (8304, bottom left, and 8305, bottom right). Co-dosing with VHL ligand 8305 demonstrates marked BRD4 degradation after 4h with sustained degradation for up to 8h after drugs are washed out. The VHL ligand, VHL298, inhibited CURE-PRO mediated degradation.

FIGS. 73A-73B depict the dependence of the proteasome for CURE-PRO-mediated BRD4 degradation, as detected on a WES capillary electrophoresis instrument (Proteinsimple) (FIG. 73A), and the structures (FIG. 73B) of the reversibly binding ligands, with the BRD ligand (BRD-E20, top), and VHL binding ligands (8304, bottom left) and 8305 (bottom right). Co-dosing with VHL ligand 8305 demonstrates marked BRD4 degradation that is inhibited with the proteasome inhibitors, MG-132 and Carfilzomib.

FIGS. 74A-74B depict the inhibition of CURE-PRO-mediated BRD4 degradation, as detected by Proteinsimple (FIG. 74A) and methods described above. The preincubation of VHL298 at equimolar final concentrations with BRD-E50 and the VHL CURE-PRO ligand, 8305, inhibits the degradation of BRD4. The structures (FIG. 74B) of the reversibly binding ligands are shown with the BRD4 ligand (BRD-E50, top left) and the VHL binding ligands (8305, top right), and the self-assembled dimer (bottom) are shown.

FIGS. 75A-75B depict the dependence of the proteasome for CURE-PRO-mediated BRD4 degradation, as detected on a WES capillary electrophoresis instrument (Proteinsimple) (FIG. 75A), and the structures (FIG. 75B) of the reversibly binding ligands, with the BRD ligand (BRD-E2, top left), and the VHL binding ligand (8305, top right), and the self-assembled dimer (bottom) are shown. Co-dosing with VHL ligand 8305 demonstrates marked BRD4 degradation that is inhibited with the proteasome inhibitors, MG-132 and Carfilzomib.

FIGS. 76A-76B depict the CURE-PRO-mediated caspase activation, as detected by Caspase-Glo® 3/7 Assay System (Promega). Co-dosing BRD-E50 ligands with the VHL ligand 8305 demonstrates marked caspase activation in MOLM13 cells (FIG. 76A) and Namalwa cells (FIG. 76B), but not with monomers alone. Co-treatment for BRD-E50 with the VHL ligand, VHL298, does not increase caspase activity in either cell line.

FIGS. 77A-77B depict the CURE-PRO-mediated loss in cell viability, as detected by the CelltitreGlo® 3/7 Assay (Promega). Co-dosing BRD-E50 ligands with the VHL ligand 8305 demonstrates a greater loss of cell viability in MOLM13 cells after 24h (FIG. 77A) and 72h (FIG. 77B), with some loss in viability with the monomers alone. Co-treatment for BRD-E50 with the VHL ligand, VHL298, decreases levels of cell viability to similar levels of BRD-E50 treatment alone.

FIG. 78 is a photograph and schematic representation of an Alizarin Red optical reporter system to determine the relative binding affinities of 8 aromatic boronic acids (ABA). Chemicals were dissolved in 100% DMSO at 100 mM concentrations. Serial dilutions (from 30 mM to 0.01 mM) of the boronic acid was made into 0.1 mM Alizarin Red S. (ARS) in 0.1M phosphate buffer, pH 7.4. At higher concentrations of ABA, the ARS changed colors.

FIG. 79 is the absorbance plot from 350 nm to 750 nm of serial dilutions of 2-(hydroxymethyl)phenylboronic acid, row B from the experimental result shown in FIG. 78.

FIG. 80 is the absorbance plot from 350 nm to 750 nm of serial dilutions of 3,5-difluorophenylboronic acid, row G from the experimental result shown in FIG. 78.

FIG. 81 is a photograph and schematic representation of an Alizarin Red optical reporter system to determine the relative binding affinities of cis-diols, aromatic cis-diols, and salicylamide derivatives to an aromatic boronic acid. Chemicals were dissolved in 100% DMSO at 100 mM concentrations. 2 mM of the benzofuran-2-boronic acid was mixed with 0.1 mM ARS in 0.1M phosphate buffer, pH 7.4, and then serial dilutions (from 30 mM to 0.1 mM) of the cis-diols, aromatic cis-diols, and salicylamide derivatives were made. In these experiments, the benzofuran-2-boronic acid was in 20-fold excess over ARS, so it completely changed color, but then the diols were added at an even higher concentration, where they compete the ABA away from ARS, so the ARS turns back to its original color.

FIG. 82 is the absorbance plot from 350 nm to 750 nm of serial dilutions of catechol, row B from the experimental result shown in FIG. 81.

FIG. 83 is the absorbance plot from 350 nm to 750 nm of serial dilutions of 2,6-dihydroxybenzamide, row H from the experimental result shown in FIG. 81.

FIG. 84 is a summary of average calculated K_{eq} for various aromatic boronic acids in the Alizarin Red optical reporter system.

FIGS. 85A-C are summaries of average calculated K_{eq}2 for various diols, α-hydroxy carboxylic acids, α-hydroxyketones and other partners to a variety of boronic acids (phenylboronic acid, furan-2-boronic acid, 2-(hydroxymethyl)phenylboronic acid, benzofuran-2-boronic acid, benzothiophene-2-boronic acid, 2-fluorophenylboronic acid, 3,5-difluorophenylboronic acid, and (5-amino-2-hydroxymethylphenyl)boronic acid, HCl, dehydrate) in the Alizarin Red optical reporter system.

### DETAILED DESCRIPTION

One aspect of the present application relates to a therapeutic composition comprising:
a first precursor compound having the chemical structure:

E3ULB—C₁-L₁,

or a pharmaceutically acceptable salt, enantiomer, stereoisomer, solvate, or polymorph thereof, and
a second precursor compound having the chemical structure:

TPB—C₂-L₂,

or a pharmaceutically acceptable salt, enantiomer, stereoisomer, solvate, or polymorph thereof, wherein:
E3ULB is a small molecule E3 ubiquitin ligase binding moiety that binds an E3 ubiquitin ligase, an E3 ubiquitin ligase complex, or subunit thereof,
TPB is a small molecule comprising a BET domain protein binding moiety,
C₁ and C₂ are independently a bond or a connector element,
L₁ and L₂ are linker element pairs suitable for binding to one another by two or more reversible covalent bonds that form under physiological conditions, each linker element having a molecular weight of 54 to 420 Daltons, said linker element pairs being selected from the group consisting of:
(1) one linker element comprising an aromatic 1,2-diol-containing moiety and the other linker element comprising an aromatic or heteroaromatic boronic acid- or boronic ester-containing moiety;
(2) one linker element comprising an aromatic 1,2-carbonyl and alcohol-containing moiety and the other linker element comprising an aromatic or heteroaromatic boronic acid- or boronic ester-containing moiety;
(3) one linker element comprising a cis-dihydroxycoumarin-containing moiety and the other linker element comprising an aromatic or heteroaromatic boronic acid- or boronic ester-containing moiety;
(4) one linker element comprising an α-hydroxycarboxylic acid-containing moiety and the other linker element comprising an aromatic or heteroaromatic boronic acid- or boronic ester-containing moiety;
(5) one linker element comprising an aromatic 1,3-diol-containing moiety and the other linker element comprising an aromatic or heteroaromatic boronic acid- or boronic ester-containing moiety;
(6) one linker element comprising an aromatic 2-(aminomethyl)phenol-containing moiety and the other linker element comprising an aromatic or heteroaromatic boronic acid- or boronic ester- or 1,2-boronic acid and carbonyl-containing moiety;
(7) one linker element comprising a cis-1,2-diol-, or cis-1,3-diol-, or a ring system comprising a trans-1,2-diol-containing moiety and the other linker element comprising an aromatic or heteroaromatic boronic acid- or boronic ester-containing moiety;
(8) one linker element comprising a [2.2.1] bicyclic ring system comprising a cis-1,2-diol-, or a cis-1,2-diol and cis-1,3-diol-, or a cis-1,2-diol and a β-hydroxyketone-containing moiety and the other linker element comprising an aromatic or heteroaromatic boronic acid- or boronic ester-containing moiety;
(9) one linker element comprising a [2.2.1] bicyclic ring system comprising a cis-1,2-diol- and cis-1,2-aminoalcohol-, or a cis-1,2-diol and cis-1,3-aminoalcohol-, or a cis-1,2-diol and cis-1,2-hydrazine-alcohol-containing moiety and the other linker element comprising an aromatic or heteroaromatic boronic acid- or 1,2-boronic acid and carbonyl-containing moiety;
(10) one linker element comprising a [2.2.1] bicyclic ring system comprising a cis-1,2-aminoalcohol and cis-1,3-diol- or a cis-1,2-aminoalcohol and a β-hydroxyketone-containing moiety and the other linker element comprising an aromatic or heteroaromatic boronic acid- or 1,2-boronic acid and carbonyl-containing moiety;
(11) one linker element comprising a cis-1,2-aminoalcohol-, or a ring system comprising a trans-1,2-aminoalcohol-containing moiety and the other linker element comprising an aromatic or heteroaromatic boronic acid- or boronic ester- or 1,2-boronic acid and carbonyl-containing moiety;
(12) one linker element comprising a cis-1,3-aminoalcohol-containing moiety and the other linker element comprising an aromatic or heteroaromatic boronic acid- or boronic ester- or 1,2-boronic acid and carbonyl-containing moiety;
(13) one linker element comprising an acyl or aromatic hydrazine-containing moiety and the other linker molecule comprising an aromatic or heteroaromatic 1,2-boronic acid and carbonyl-containing moiety; and
(14) one linker element comprising an α-hydroxyketone-containing moiety and the other linker molecule comprising an α-hydroxyketone-containing moiety.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs.

As used herein, the term “halogen” means fluoro, chloro, bromo, or iodo.

The term “alkyl” means an aliphatic hydrocarbon group which may be straight or branched having about 1 to about 6 carbon atoms in the chain (or the number of carbons designated by “Cn-Cn”, where n is the numerical range of carbon atoms). Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkoxy” means groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

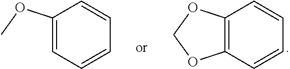

The term “aryl” means an aromatic monocyclic or multi-cyclic (polycyclic) ring system of 6 to about 19 carbon atoms, preferably of 6 to about 10 carbon atoms, and includes arylalkyl groups. The ring system of the aryl group may be optionally substituted. Representative aryl groups of the present application include, but are not limited to, groups such as phenyl, naphthyl, azulenyl, phenanthrenyl, anthracenyl, fluorenyl, pyrenyl, triphenylenyl, chrysenyl, and naphthacenyl.

The term “heteroaryl” means an aromatic monocyclic or multi-cyclic ring system of about 5 to about 19 ring atoms, or about 5 to about 10 ring atoms, in which one or more of the atoms in the ring system is/are element(s) other than carbon, for example, nitrogen, oxygen, or sulfur. In the case of multi-cyclic ring system, only one of the rings needs to be aromatic for the ring system to be defined as “heteroaryl.” Particular heteroaryls contain about 5 to 6 ring atoms. The prefix aza, oxa, thia, or thio before heteroaryl means that at least a nitrogen, oxygen, or sulfur atom, respectively, is present as a ring atom. A nitrogen, carbon, or sulfur atom in the heteroaryl ring may be optionally oxidized; the nitrogen may optionally be quaternized. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

The term “carbocycle” means a non-aromatic, saturated or unsaturated, mono- or multi-cyclic ring system of about 3 to about 8 carbon atoms. Exemplary carbocyclic groups include, without limitation, cyclopropyl, cyclobutyl, cyclopentyl, cyclohexyl, and cycloheptyl.

As used herein, “heterocycle” refers to a stable 3- to 18-membered ring (radical) of carbon atoms and from one to five heteroatoms selected from nitrogen, oxygen, and sulfur. The heterocycle may be a monocyclic or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone.

Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “alkyl amine” means groups of from 1 to 8 carbon atoms of a straight, branched, or cyclic configuration, and combinations thereof, which contains a nitrogen within, or at the end of the carbon chain. The nitrogen can further be substituted with additional carbon subtiuents.

The term “substituted” specifically envisions and allows for one or more substitutions that are common in the art. However, it is generally understood by those skilled in the art that the substituents should be selected so as to not adversely affect the useful characteristics of the compound or adversely interfere with its function. Suitable substituents may include, for example, halogen groups, perfluoroalkyl groups, perfluoroalkoxy groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, amino groups, alkyl- and dialkylamino groups, carbamoyl groups, alkylaminocarbonyl groups, dialkylamino carbonyl groups, arylcarbonyl groups, aryloxycarbonyl groups, alkylsulfonyl groups, arylsulfonyl groups, cycloalkyl groups, cyano groups, C₁-C₆ alkylthio groups, arylthio groups, nitro groups, boronate or boronyl groups, phosphate or phosphonyl groups, sulfamyl groups, sulfonyl groups, sulfinyl groups, and combinations thereof. In the case of substituted combinations, such as “substituted arylalkyl,” either the aryl or the alkyl group may be substituted, or both the aryl and the alkyl groups may be substituted with one or more substituents. Additionally, in some cases, suitable substituents may combine to form one or more rings as known to those of skill in the art.

According to one embodiment, the compounds of the present application are unsubstituted. “Unsubstituted” atoms bear all of the hydrogen atoms dictated by their valency.

According to another embodiment, the compounds of the present application are substituted. By “substituted” it is meant that a group may have a substituent at each substitutable atom of the group (including more than one substituent on a single atom), provided that the designated atom's normal valency is not exceeded, and the identity of each substituent is independent of the others. For example, up to three H atoms in each residue are replaced with substituents such as halogen, haloalkyl, hydroxy, loweralkoxy, carboxy, carboalkoxy (also referred to as alkoxycarbonyl), carboxamido (also referred to as alkylaminocarbonyl), cyano, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. When a substituent is keto (i.e., =0), then two hydrogens on the atom are replaced. Combinations of substituents and/or variables are permissible only if such combinations result in stable compounds; by “stable compound” it is meant a compound that is sufficiently robust to survive isolation to a useful degree of purity from a reaction mixture, and formulation into an agent intended for a suitable use.

By “compound(s) of the application” and equivalent expressions, it is meant compounds herein described, which expression includes the prodrugs, the pharmaceutically acceptable salts, the oxides, and the solvates, e.g. hydrates, where the context so permits.

Compounds described herein may contain one or more asymmetric centers and may thus give rise to enantiomers, diastereomers, and other stereoisomeric forms. Each chiral center may be defined, in terms of absolute stereochemistry, as (R)- or (S)—. The present application is meant to include all such possible isomers, as well as mixtures thereof, including racemic and optically pure forms. Optically active (R)- and (S)—, (−)- and (+)-, or (D)- and (L)-isomers may be prepared using chiral synthons or chiral reagents, or resolved using conventional techniques. All tautomeric forms are also intended to be included.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the present application, a compound as described herein, including in the contexts of pharmaceutical compositions, methods of treatment, and compounds per se, is provided as the salt form.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

The term “pharmaceutically acceptable salt” refers to salts prepared from pharmaceutically acceptable non-toxic acids or bases including inorganic acids and bases and organic acids and bases. Suitable pharmaceutically acceptable acid addition salts for the compounds described herein include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds described herein include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine, and tris (hydroxymethyl) aminomethane; alkali metal salts, such as but not limited to lithium, potassium, and sodium; alkali earth metal salts, such as but not limited to barium, calcium, and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids, and boronic acids. Pharmaceutical acceptable enol ethers include, but are not limited to, derivatives of formula C═C (OR) where R is hydrogen, alkyl, alkenyl, alkynyl, aryl, heteroaryl, cycloalkyl, or heterocyclyl. Pharmaceutically acceptable enol esters include, but are not limited to, derivatives of formula C═C(OC(O) R) where R is hydrogen, alkyl, alkenyl, alkynyl, aryl, heteroaryl, cycloalkyl, or heterocyclyl. Pharmaceutical acceptable solvates and hydrates are complexes of a compound with one or more solvent or water molecules, or 1 to about 100, or 1 to about 10, or one to about 2,3 or 4, solvent or water molecules.

The term “method of treating” means amelioration or relief from the symptoms and/or effects associated with the disorders described herein. As used herein, reference to “treatment” of a patient is intended to include prophylaxis.

The term “reversible covalent bonds” refers to reversible or labile bonds which may be selected from the group comprising: physiologically labile bonds, cellular physiologically labile bonds, pH labile bonds, very pH labile bonds, and extremely pH labile bonds.

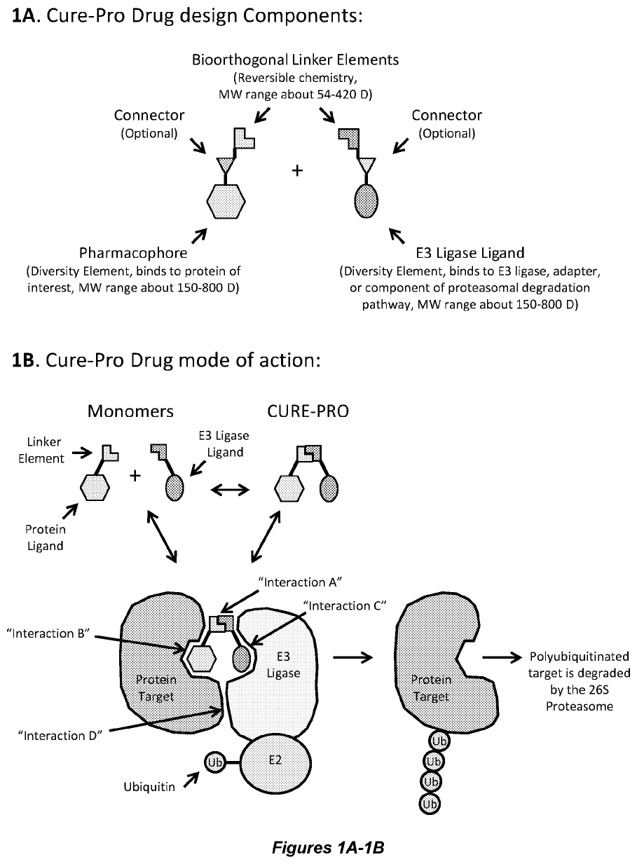

In one embodiment of the present application, the pharmacophore recruits the target protein and the ligand recruits an E3 ubiquitin ligase (or adaptor protein as part of the E3 ligase machinery) together, resulting in proximity-mediated ubiquitination (via an E2 ubiquitin-conjugating enzyme) and subsequent protein degradation by the 26S Proteasome (FIG. 1B). Central to the success of this approach is designing the pharmacophore, ligand, (optional) connector lengths, and linkers to enable additional interactions between the target protein and the E3 ubiquitin ligase machinery. Thus, for optimum activity and selectivity, the basic CURE-PRO design encompasses four binding interactions: (Interaction A) Pharmacophore linker to ligand linker; (Interaction B) Pharmacophore to target protein; (Interaction C) Ligand to E3 ligase or ligase-machinery; and (Interaction D) Target protein to E3 ligase or ligase machinery (see FIG. 1B). While the dissociation constant of any single of these interactions may be in the 1 μM to 100 μM range, combined they can create a highly effective therapeutic that works at nanomolar concentrations. Since the CURE-PRO molecules effect the target protein through catalytic degradation, therapeutic efficacy may be achieved as long as the rate of target protein degradation exceeds the rate of re-synthesis. Further, as exhibited for PROTACs using a promiscuous ATP binding site pharmacophore, the protein kinase with the tightest affinity to the pharmacophore is not always the most highly degraded, reaffirming the conclusion that target protein—E3 ligase tertiary interactions play a crucial role in the overall efficacy of the molecule (Bondenon et al., Cell Chem. Biol. 25(1):78-87 (2018); Huang et al., Cell Chem. Biol. 25(1):88-99.e6 (2018), which are hereby incorporated by reference in their entirety).

One aspect of the present application is directed to a therapeutic composition, comprising of two precursor compounds (monomers) that are suitable for assembly via two or more reversible covalent bonds. The monomer is a polyfunctionalized molecule comprising a bioorthogonal linker element and ligand or pharmacophore, wherein the linker and ligand/pharmacophore are covalently coupled to each other either directly or through an optional connector moiety. The monomer comprises of:
1) bioorthogonal linker element having the generic structure:

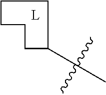 [SMILES: *C1CCCCC1]

2) optional connector moiety having the general structure:

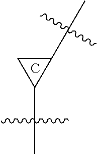

and 3) ligand or pharmacophore having the general structure:

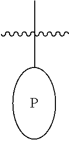

where the lines crossed with a dashed line illustrate the one or more bonds formed joining the linkers, pharmacophores, or ligands to each other directly or through a connector. The pharmacophore (or ligand) moiety may bind to the target protein (TPB, which may be, for example, a small molecule comprising a BET domain protein binding moiety or some other moiety) or E3 ubiquitin ligase or ligase machinery (i.e., E3ULB). While each monomer is depicted in the figures or text as a linear connection of “pharmacophore-connector-linker” (i.e., E3ULB-C₁-L₁, or TPB-C₂-L₂), the pharmacophore (or ligand) may comprise of a portion of the linker or connector, and the linker or connector may comprise of a portion of the pharmacophore (or ligand). Thus, a given monomer always comprises of a pharmacophore (or ligand) moiety and a linker element, but certain moieties or structures within the monomer may play dual roles as both pharmacophore (or ligand) moiety and linker element, which are coupled through one or more chemical bonds or connectors. Further, either of the pharmacophores (or ligands), connectors, or linker elements of the individual or assembled monomers may have additional interactions with the target protein (TPB) or E3 ubiquitin ligase or ligase machinery (E3ULB) to facilitate or stabilize formation of the quaternary complex.

### Linkers

Linker elements have a molecular weight of about 54 to 420 Daltons and have a dissociation constant of less than 300 μM under physiological conditions. Linker elements form reversible covalent bonds to their partner(s) and may have dissociation constants up to 1 M in aqueous solutions.

In a first embodiment of the therapeutic composition of the present application, one of the linker elements, L₁ or L₂, is derived from an aromatic 1,2-diol-containing compound comprising the following structure, or salt, enantiomer, stereoisomer, or polymorph thereof.

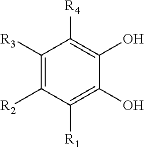

wherein
R₁ to R₄ are independently —H, —OH, —C₁₋₆ alkyl, —C₁₋₆ alkoxy, alkyl amine, —C(O)NH₂, —CN, aryl, heteroaryl, an electron donating moiety, or a bond to —C₁-E3ULB or —C₂-TPB; wherein when two of R₁ to R₄ are adjacent they may optionally be taken together to form one or more fused 5- or 6-membered aromatic, heteroaromatic, carbocyclic, or heterocyclic rings; and wherein one of R₁ to R₄ comprises a bond to —C₁-E3ULB or —C₂-TPB;
and the other linker element, L₂ or L₁ respectively, is derived from an aromatic or heteroaromatic boronic acid- or boronic ester-containing compound is comprised of one of the following structures, or salts, enantiomers, stereoisomers, or polymorphs thereof:

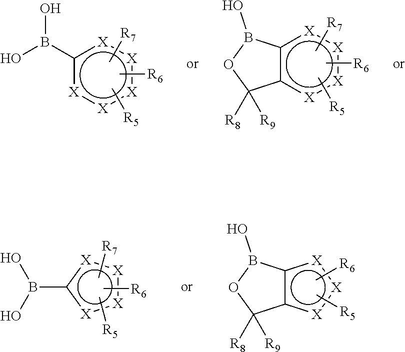

wherein
R₅ to R₇ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₈ and R₉ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₅ to R₇ are adjacent they may optionally be taken together to form one or more fused 5- or 6-membered aromatic, heteroaromatic, carbocyclic, or heterocyclic rings; and one of R₅ to R₇ comprises a bond to —C₂-TPB, or one of R₅ to R₇ comprises a bond to —C₁-E3ULB.

In accordance with the first embodiment of the linkers of the present application, the one of the linker elements L₁ or L₂ is comprised of one of the following structures, or salts, enantiomers, stereoisomers, or polymorphs thereof:

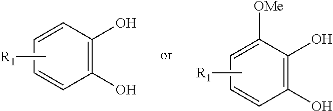

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

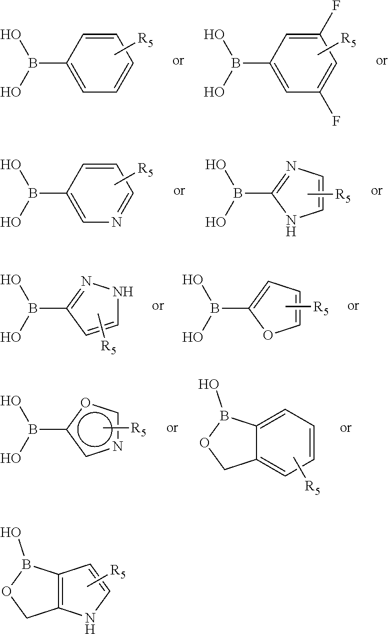

wherein
R₅ is a bond to either —C₁-E3ULB or —C₂-TPB.

In a second embodiment of the linkers of the therapeutic composition of the present application, one of the linker elements L₁ or L₂ is derived from an aromatic 1,2-carbonyl and alcohol-containing compound comprising the following structure, or salt, enantiomer, stereoisomer, or polymorph thereof:

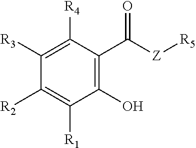

wherein
R₁ to R₄ are independently —H, —OH, —C₁₋₆ alkyl, —C₁₋₆ alkoxy, alkyl amine, —C(O)NH₂, —CN, aryl, heteroaryl, an electron donating moiety, an acyl, or a bond to —C₁-E3ULB or —C₂-TPB;
R₅ is —H, —OH, —C₁₋₆ alkoxy, —OPh, or a bond to —C₁-E3ULB or —C₂-TPB; and
Z is O or NH;
   wherein when two of R₁ to R₄ are adjacent they may optionally be taken together to form one or more fused 5- or 6-membered aromatic, heteroaromatic, carbocyclic, or heterocyclic rings; and wherein one of R₁ to R₅ comprises a bond to —C₁-E3ULB, or one of R₁ to R₅ comprises a bond to —C₂-TPB;
and the other linker element, L₂ or L₁, respectively, is derived from an aromatic or heteroaromatic boronic acid- or boronic ester-containing compound comprising of one of the following structures, or salts, enantiomers, stereoisomers, or polymorphs thereof:

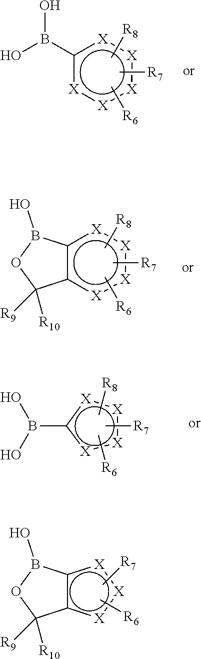

wherein
R₆ to R₈ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₉ and R₁₀ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
wherein when two of R₆ to R₈ are adjacent they may optionally be taken together to form one or more fused 5- or 6-membered aromatic, heteroaromatic, carbocyclic, or heterocyclic rings; and one of R₆ to R₈ comprises a bond to —C₂-TPB, or one of R₆ to R₈ comprises a bond to —C₁-E3ULB.

In accordance with the second embodiment of the linkers of the present application, one of the linker elements L₁ or L₂ comprises the following structure, or salt, enantiomer, stereoisomer, or polymorph thereof:

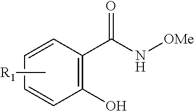

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

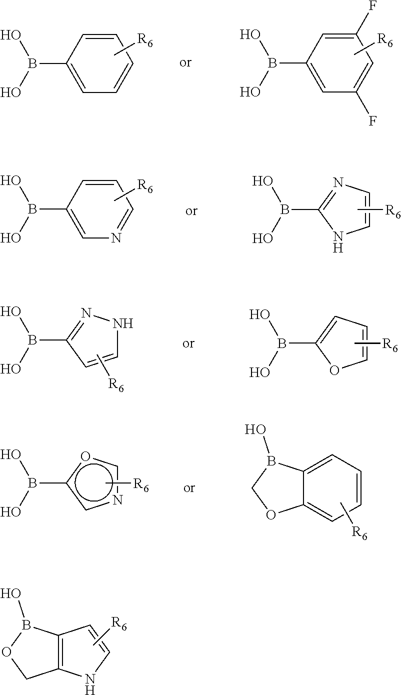

wherein
R₆ is a bond to either —C₁-E3ULB or —C₂-TPB.

In a third embodiment of the linkers of the therapeutic composition of the present application, one of the linker elements L₁ or L₂ is derived from a cis-dihydroxycoumarin-containing compound comprising of the following structure, or salt, enantiomer, stereoisomer, or polymorph thereof:

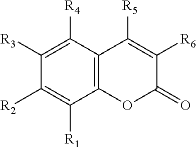

wherein
R₁ to R₆ are independently —H, —OH, —C₁₋₆ alkyl, —C₁₋₆ alkoxy, alkyl amine, aryl, heteroaryl, —C(O)NH₂, —CN, an electron donating moiety, an acyl, or bond to —C₁-E3ULB or —C₂-TPB;
   wherein at least two consecutive Rgroups within R₁ to R₄ are —OH; and wherein one of R₁ to R₆ comprises a bond to —C₁-E3ULB or —C₂-TPB;
and the other linker element, L₂ or L₁, respectively, is derived from an aromatic or heteroaromatic boronic acid- or boronic ester-containing compound comprising of one of the following structures, or salts, enantiomers, stereoisomers, or polymorphs thereof:

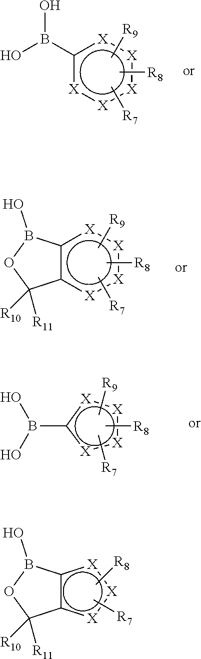

wherein
R₇ to R₉ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₁₀ and R₁₁ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
wherein when two of R₇ to R₉ are adjacent they may optionally be taken together to form one or more fused 5- or 6-membered aromatic, heteroaromatic, carbocyclic, or heterocyclic rings; and one of R₇ to R₉ comprises a bond to —C₂-TPB, or one of R₇ to R₉ comprises a bond to —C₁-E3ULB.

In accordance with the third embodiment of the linkers of the present application, one of the linker elements L₁ or L₂ comprises the following structure, or salt, enantiomer, stereoisomer, or polymorph thereof:

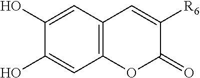

wherein
R₆ is a bond to either-C₁-E3ULB or —C₂-TPB;
and the other linker element, L₂ or L₁, respectively, is comprised of one of the following structures, or salts, enantiomers, stereoisomers, or polymorphs thereof:

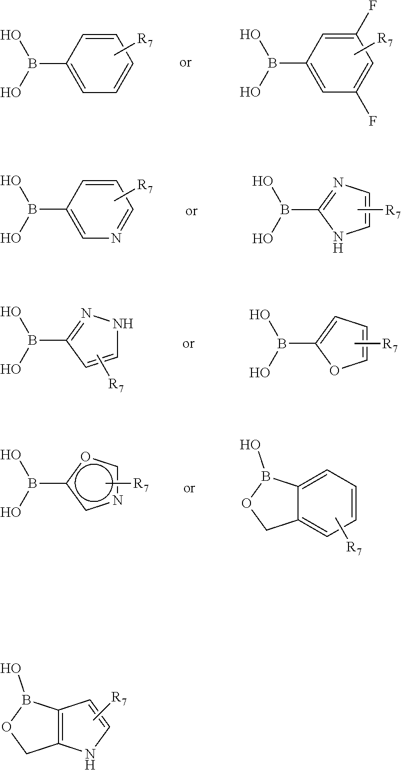

wherein
R₇ is a bond to either —C₁-E3ULB or —C₂-TPB.

In a fourth embodiment of the linkers of the present application, one of the linker elements L₁ or L₂ is derived from an α-hydroxycarboxylic acid-containing compound comprised of the following structure, or salt, enantiomer, stereoisomer, or polymorph thereof:

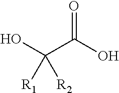

wherein
R₁ and R₂ are independently —H, —OH, —C₁₋₆ alkyl, —C₁₋₆ alkoxy, alkyl amine, —C₁₋₆ cycloalkyl, aryl, heteroaryl, a bond to —C₁-E3ULB or —C₂-TPB, or can be connected to each other via a spiro 3-, 4-, 5-, or 6-membered ring;
   wherein one of R₁ and R₂ comprises a bond to —C₁-E3ULB or —C₂-TPB;
and the other linker element, L₂ or L₁, respectively, is derived from an aromatic or heteroaromatic boronic acid- or boronic ester-containing compound comprising of one of the following structures, or salts, enantiomers, stereoisomers, or polymorphs thereof:

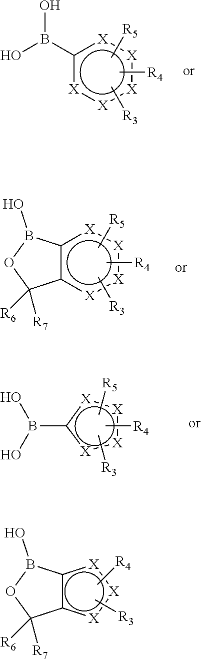

wherein
R₃ to R₅ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₃ to R₅ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₃ to R₅ are adjacent they may optionally be taken together to form one or more fused 5- or 6-membered aromatic, heteroaromatic, carbocyclic, or heterocyclic rings; and one of R₃ to R₅ comprises a bond to —C₂-TPB, or one of R₃ to R₅ comprises a bond to —C₁-E3ULB.

In accordance with the fourth embodiment of the linkers, one of the linker elements L₁ or L₂ comprises the following structure, or salt, enantiomer, stereoisomer, or polymorph thereof:

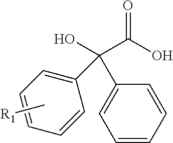

wherein
R₁ is a bond to —C₁-E3ULB or —C₂-TPB;
and the other linker element, L₂ or L₁, respectively, is comprised of one of the following structures, or salts, enantiomers, stereoisomers, or polymorphs thereof:

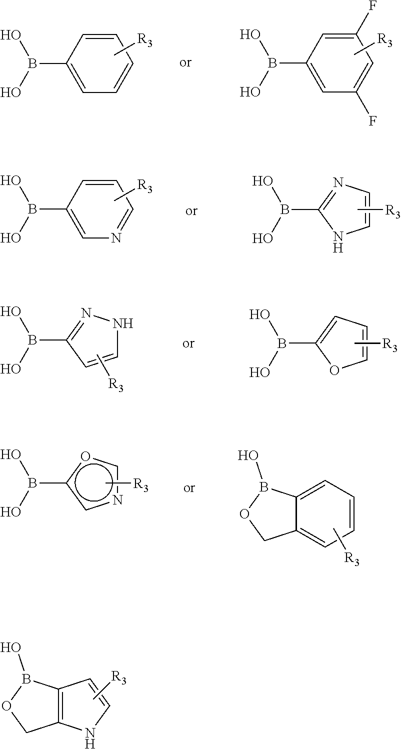

wherein
R₃ is a bond to either —C₁-E3ULB or —C₂-TPB.

In a fifth embodiment of the linkers of the present application, one of the linker elements L₁ or L₂ is derived from an aromatic 1,3-diol-containing compound comprising of the following structure, or salt, enantiomer, stereoisomer, or polymorph thereof:

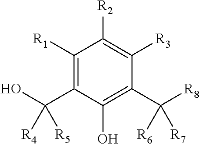

wherein
R₁ to R₃ are independently —H, —OH, —C₁₋₆ alkyl, —C₁₋₆ alkoxy, alkyl amine, -acyl, aryl, heteroaryl, —C(O)NH₂, —CN, an electron donating moiety, or a bond to —C₁-E3ULB or —C₂-TPB; wherein when two of R₁ to R₃ are adjacent they may optionally be taken together to form one or more fused 5- or 6-membered aromatic, heteroaromatic, carbocyclic, or heterocyclic rings;
R₄ to R₇ are independently —H, —C₁₋₆ alkyl, aryl, or a bond to —C₁-E3ULB or —C₂-TPB; and R₈ is —H; —OH; —C₁₋₆ alkyl, aryl, or a bond to —C₁-E3ULB or —C₂-TPB;
   wherein one of R₁ to R₅ comprises a bond to —C₁-E3ULB or —C₂-TPB;
and the other linker element, L₂ or L₁, respectively, is derived from an aromatic or heteroaromatic boronic acid- or boronic ester-containing compound comprising of one of the following structures, or salts, enantiomers, stereoisomers, or polymorphs thereof:

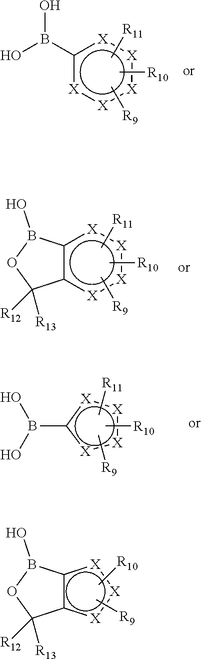

wherein
R₉ to R₁₁ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₁₂ and R₁₃ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₉ to R₁₁ are adjacent they may optionally be taken together to form one or more fused 5- or 6-membered aromatic, heteroaromatic, carbocyclic, or heterocyclic rings; and one of R₆ to R₈ comprises a bond to —C₂-TPB, or one of R₉ to R₁₁ comprises a bond to —C₁-E3ULB.

In accordance with the fifth embodiment of the linkers of the present application, one of the linker elements L₁ or L₂ comprises the following structure, or salt, enantiomer, stereoisomer, or polymorph thereof:

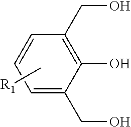

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

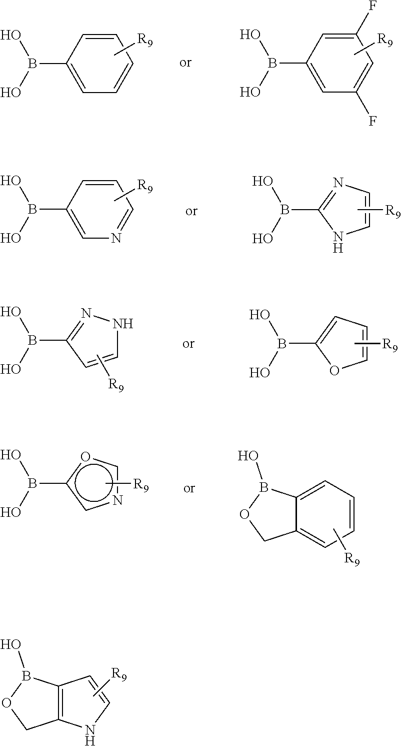

wherein
R₉ is a bond to either —C₁-E3ULB or —C₂-TPB.

In a sixth embodiment of the linkers of the present application, one of the linker elements L₁ or L₂ is derived from an aromatic 2-(aminomethyl)phenol-containing compound comprising of the following structure, or salt, enantiomer, stereoisomer, or polymorph thereof:

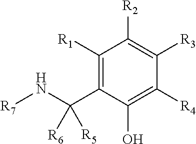

wherein
R₁ to R₄ are independently —H, —OH, —C₁₋₆ alkyl, —C₁₋₆ alkoxy, alkyl amine, -acyl, aryl, heteroaryl, —C(O)NH₂, —CN, an electron donating moiety, or a bond to —C₁-E3ULB or —C₂-TPB; wherein when two of R₁ to R₄ are adjacent they may optionally be taken together to form one or more fused 5- or 6-membered aromatic, heteroaromatic, carbocyclic, or heterocyclic rings;
R₅ to R₆ are independently —H, —C₁₋₆ alkyl, aryl, or a bond to —C₁-E3ULB or —C₂-TPB; and
R₇is —H; —OH, —C₁₋₆ alkyl, aryl, or a bond to —C₁-E3ULB or —C₂-TPB;
   wherein one of R₁ to R₇ comprises a bond to —C₁-E3ULB or —C₂-TPB;
and the other linker element, L₂ or L₁, respectively, is derived from an aromatic or heteroaromatic boronic acid- or boronic ester- or 1,2-boronic acid and carbonyl-containing compound comprising of one of the following structures, or salts, enantiomers, stereoisomers, or polymorphs thereof:

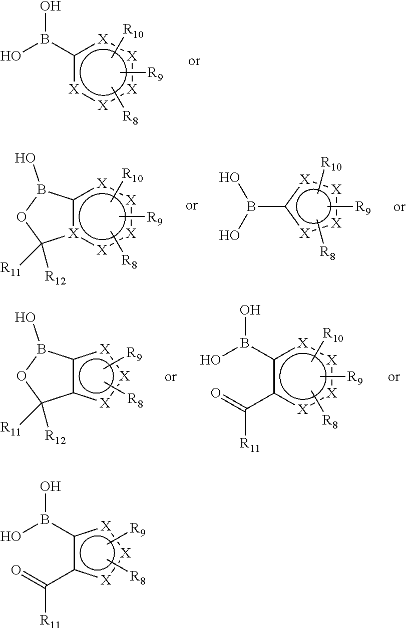

wherein
R₈ to R₁₀ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB; R₁₁ and R₁₂ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₈ to R₁₀ are adjacent they may optionally be taken together to form one or more fused 5- or 6-membered aromatic, heteroaromatic, carbocyclic, or heterocyclic rings; and one of R₈ to R₁₀ comprises a bond to —C₂-TPB, or one of R₈ to R₁₀ comprises a bond to —C₁-E3ULB.

In accordance with the sixth embodiment of the linkers of the present application, one of the linker elements L₁ or L₂ comprises the following structure, or salt, enantiomer, stereoisomer, or polymorph thereof:

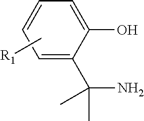

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

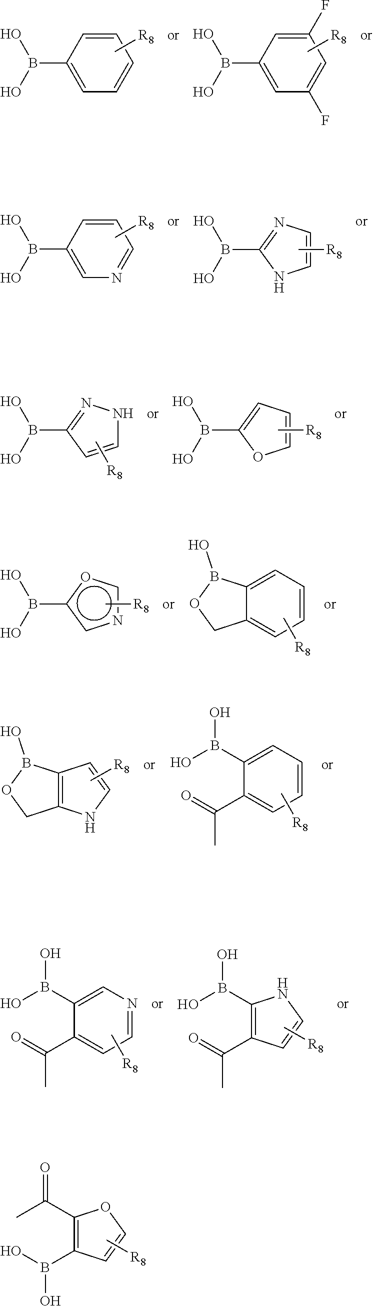

wherein
R₈ is a bond to either —C₁-E3ULB or —C₂-TPB.

In a seventh embodiment of the linkers of the present application, one of the linker elements L₁ or L₂ is derived from a cis-1,2-diol or cis-1,3-diol-, or a ring system comprising a trans-1,2-diol-containing compound and is comprised of one of the following structures, or salts, enantiomers, stereoisomers, or polymorphs thereof:

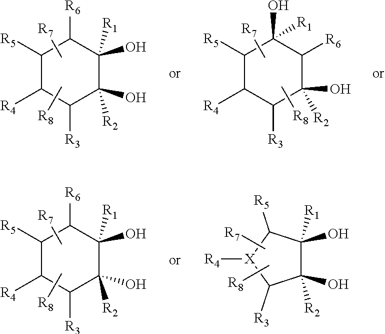

wherein
R₁ and R₂ are independently —H, —C₁₋₆ alkyl, —C₁₋₆ alkoxy, aryl, heteroaryl, or or a bond to —C₁-E3ULB R₁ or —C₂-TPB;
R₃ to R₈ are independently —H, —OH, —NH₂, —C₁₋₆ alkyl, —C₁₋₆ alkoxy, aryl, heteroaryl, —NHMe, —NMe₂, or a bond to —C₁-E3ULB R₁ or —C₂-TPB;
X is independently C or N; and
   wherein R₇ and R₈ can optionally be connected to each other to form [3.1.1], [2.2.1], and [2.2.2]bicyclic ring systems, such that the hydroxyls are cis to each other; and wherein one of R₁ to R₈ independently comprises a bone to —C₁-E3ULB or —C₂-TPB;
and the other linker element, L₂ or L₁, respectively, is derived from an aromatic or heteroaromatic boronic acid- or boronic ester-containing compound comprising of one of the following structures, or salts, enantiomers, stereoisomers, or polymorphs thereof:

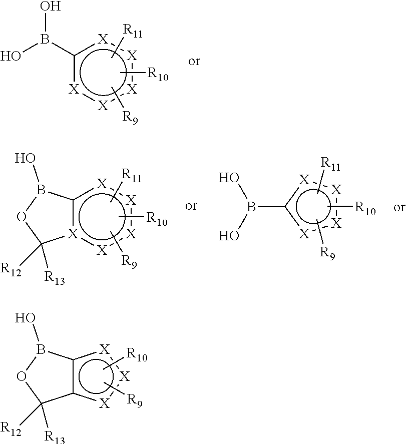

wherein
R₉ to R₁₁ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₁₂ and R₁₃ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₉ to R₁₁ are adjacent they may optionally be taken together to form one or more fused 5- or 6-membered aromatic, heteroaromatic, carbocyclic, or heterocyclic rings; and one of R₉ to R₁₁ comprises a bond to —C₂-TPB, or one of R₉ to R₁₁ comprises a bond to —C₁-E3ULB.

In accordance with the seventh embodiment of the linkers of the present application, one of the linker elements L₁ or L₂ is comprised of one of the following structures, or salts, enantiomers, stereoisomers, or polymorphs thereof:

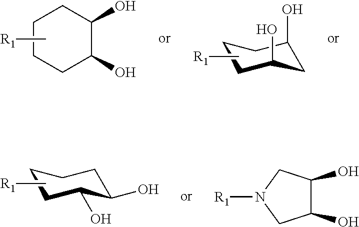

wherein
R₁ is a bond to either —C₁-E3ULB or —C₂-TPB.
and the other linker element, L₂ or L₁, respectively, is comprised of one of the following structures, or salts, enantiomers, stereoisomers, or polymorphs thereof:

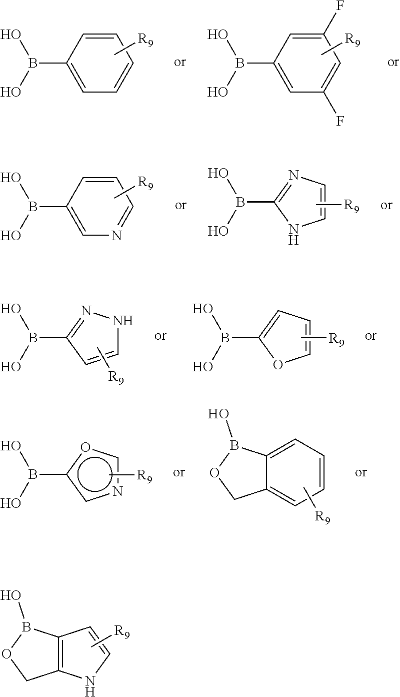

wherein
R₉ is a bond to either —C₁-E3ULB or —C₂-TPB.

In an eighth embodiment of the linkers of the present application, one of the linker elements L₁ or L₂ is derived from a [2.2.1] bicyclic ring system comprising a cis-1,2-diol, or a cis-1,2-diol and cis-1,3-diol, or a cis-1,2-diol and a β-hydroxyketone-containing compound comprising of the following structure, or salt, enantiomer, stereoisomer, or polymorph thereof:

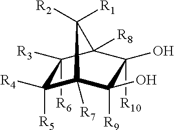

wherein
R₁ to R₅ are independently —H, —OH, —C₁₋₆ alkyl, —C₁₋₆ alkoxy, aryl, heteroaryl, or a bond to —C₁-E3ULB or —C₂-TPB; and
R₉ and R₁₀ are independently —H, —C₁₋₆ alkyl, —C₁₋₆ alkoxy, aryl, heteroaryl, or bond to —C₁-E3ULB or —C₂-TPB;
wherein R₁ and R₂ are optionally oxygen, thus forming a ketone; and wherein one of R₁ to R₁₀ comprises a bond to —C₁-E3ULB or —C₂-TPB;

and the other linker element, L₂ or L₁, respectively, is derived from an aromatic or heteroaromatic boronic acid- or boronic ester-containing compound comprising of one of the following structures, or salts, enantiomers, stereoisomers, or polymorphs thereof:

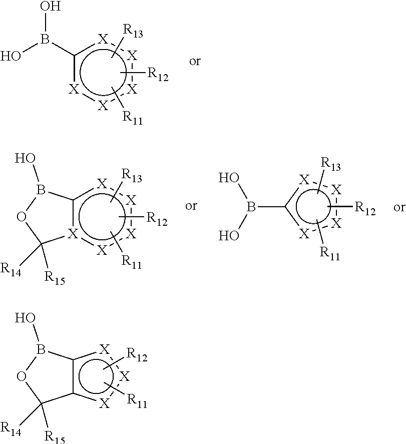

wherein
R₁₁ to R₁₃ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₁₄ and R₁₅ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₁₁ to R₁₃ are adjacent they may optionally be taken together to form one or more fused 5- or 6-membered aromatic, heteroaromatic, carbocyclic, or heterocyclic rings; and one of R₁₁ to R₁₃ comprises a bond to —C₂-TPB, or one of R₁₁ to R₁₃ comprises a bond to —C₁-E3ULB.

In accordance with the eighth embodiment of the linkers of the present application, one of the linker elements L₁ or L₂ is comprised of one of the following structures, or salts, enantiomers, stereoisomers, or polymorphs thereof:

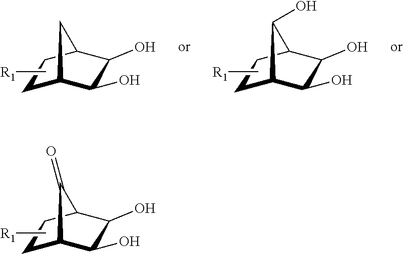

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

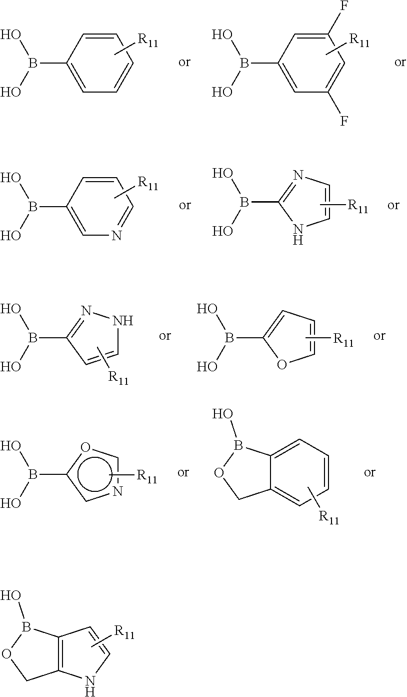

wherein
R₁₁ is a bond to either —C₁-E3ULB or —C₂-TPB.

In a ninth embodiment of the linkers of the present application, one of the linker elements L₁ or L₂ is derived from a [2.2.1] bicyclic ring system comprising a cis-1,2-diol and cis-1,2-aminoalcohol-, or a cis-1,2-diol and cis-1,3-aminoalcohol-, or a cis-1,2-diol and cis-1,2-hydrazine-alcohol-containing compound comprising of the following structure, or salt, enantiomer, stereoisomer, or polymorph thereof:

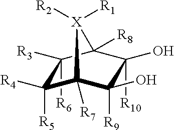

wherein
R₁ is either NH₂, NHMe, or a lone pair;
R₂ is either a lone pair, —H, —OH, —C₁₋₆ alkyl, —C₁₋₆ alkoxy, aryl, heteroaryl, or a bond to —C₁-E3ULB or —C₂-TPB;
R₃ to R₈ are independently —H, —OH, —C₁₋₆ alkyl, —C₁₋₆ alkoxy, aryl, heteroaryl, or a bond to —C₁-E3ULB or —C₂-TPB;
R₉ and R₁₀ are independently —H, —C₁₋₆ alkyl, —C₁₋₆ alkoxy, aryl, or heteroaryl, or a bond to —C₁-E3ULB or —C₂-TPB;
X is either C or N; and
   wherein one of R₂ to R₁₀ comprises a bond to —C₁-E3ULB or —C₂-TPB;
and the other linker element, L₂ or L₁, respectively, is derived from an aromatic or heteroaromatic boronic acid- or 1,2-boronic acid and carbonyl-containing compound comprising of one of the following structures, or salts, enantiomers, stereoisomers, or polymorphs thereof:

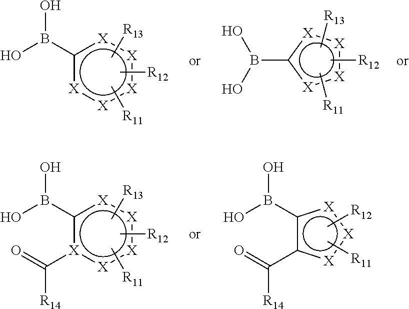

wherein
R₁₁ to R₁₃ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₁₄ is independently —H, —C₁₋₆ alkyl, aryl, heteroaryl, a bond to —C₁-E3ULB or —C₂-TPB;
X is independently C, N, O, or S; and
   wherein when two of R₁₁ to R₁₃ are adjacent they may optionally be taken together to form one or more fused 5- or 6-membered aromatic, heteroaromatic, carbocyclic, or heterocyclic rings; and one of R₁₁ to R₁₃ comprises a bond to —C₂-TPB, or one of R₁₁ to R₁₃ comprises a bond to —C₁-E3ULB.

In accordance with the ninth embodiment of the linkers of the present application, one of the linker elements L₁ or L₂ is comprised of one of the following structures, or salts, enantiomers, stereoisomers, or polymorphs thereof:

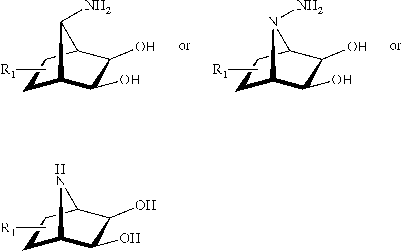

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

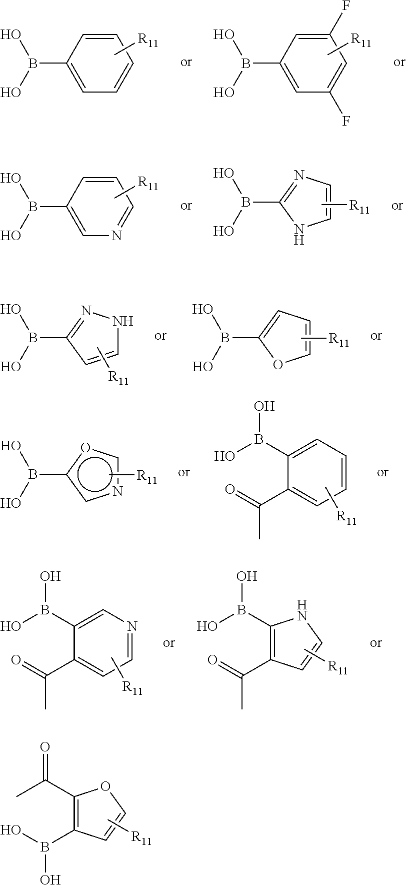

wherein
R₁₁ is a bond to either —C₁-E3ULB or —C₂-TPB.

In a tenth embodiment of the linkers of the present application, one of the linker elements L₁ or L₂ is derived from a [2.2.1] bicyclic ring system comprising a cis-1,2-aminoalcohol and cis-1,3-diol- or a cis-1,2-aminoalcohol and a β-hydroxyketone-containing compound comprising of the following structure, or salt, enantiomer, stereoisomer, or polymorph thereof:

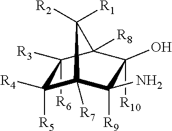

wherein
R₁ and R₂ are optionally oxygen, thus forming a ketone, or R₁ is OH
R₂ to R₈ are independently —H, —OH, —C₁₋₆ alkyl, —C₁₋₆ alkoxy, aryl, heteroaryl, or a bond to —C₁-E3ULB or —C₂-TPB; and
R₉ and R₁₀ are independently —H, —C₁₋₆ alkyl, —C₁₋₆ alkoxy, aryl, or heteroaryl; wherein one of R₂ to R₁₀ comprises a bond to —C₁-E3ULB or —C₂-TPB;
and the other linker element, L₂ or L₁, respectively, is derived from an aromatic or heteroaromatic boronic acid- or 1,2-boronic acid and carbonyl-containing compound comprised of one of the following structures, or salts, enantiomers, stereoisomers, or polymorphs thereof:

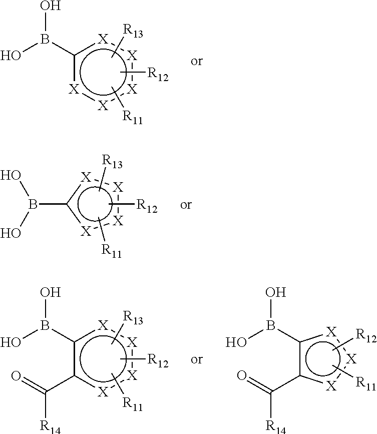

wherein
R₁₁ to R₁₃ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₁₄ is independently —H, —C₁₋₆ alkyl, aryl, heteroaryl, a bond to —C₁-E3ULB or —C₂-TPB;
X is independently C, N, O, or S; and
   wherein when two of R₁₁ to R₁₃ are adjacent they may optionally be taken together to form one or more fused 5- or 6-membered aromatic, heteroaromatic, carbocyclic, or heterocyclic rings; and one of R₁₁ to R₁₃ comprises a bond to —C₂-TPB, or one of R₁₁ to R₁₃ comprises a bond to —C₁-E3ULB.

In accordance with the tenth embodiment of the linkers of the present application, one of the linker elements L₁ or L₂ is comprised of one of the following structures, or salts, enantiomers, stereoisomers, or polymorphs thereof:

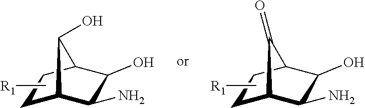

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

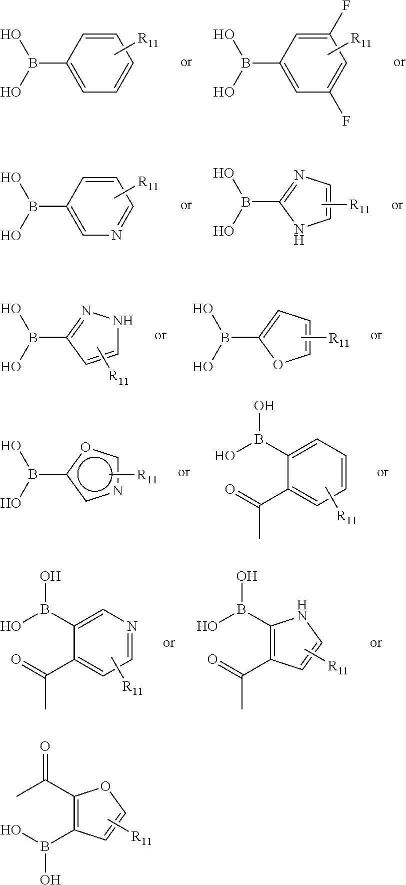

wherein
R₁₁ is a bond to either —C₁-E3ULB or 2-TPB.

In an eleventh embodiment of the linkers of the present application, one of the linker elements L₁ or L₂ is derived from a cis-1,2-aminoalcohol-, or a ring system comprising a trans-1,2-aminoalcohol-containing compound comprising of the following structure, or salt, enantiomer, stereoisomer, or polymorph thereof:

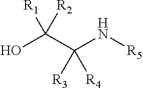

wherein
R₁ to R₄ are independently —H, —CH₂OH, —CH₂NH₂, —COOH, —CONH₂, —C₁₋₆ alkyl, —C₁₋₆ alkoxy, aryl, heteroaryl, or a bond to —C₁-E3ULB or —C₂-TPB;
R₅ is —H, —NH₂, —NHMe, —NMe₂, —CH₂COOH, —C₁₋₆ alkyl, —C₁₋₆ alkoxy, aryl, heteroaryl, or a bond to —C₁-E3ULB or —C₂-TPB;
   wherein R₁ or R₂ can optionally be connected to either R₃, R₄, or R₅ to make a ring, such that the amino and alcohol moieties are cis with respect to each other; R₃ or R₄ can optionally be connected to R₅ to make a ring, such that the amino and alcohol moieties are cis with respect to each other; and wherein one of R₁ to R₅ comprises a bond to —C₁-E3ULB or —C₂-TPB;

and the other linker element, L₂ or L₁, respectively, is derived from an aromatic or heteroaromatic boronic acid- or boronic ester- or 1,2-boronic acid and carbonyl-containing compound comprising of one of the following structures, or salts, enantiomers, stereoisomers, or polymorphs thereof:

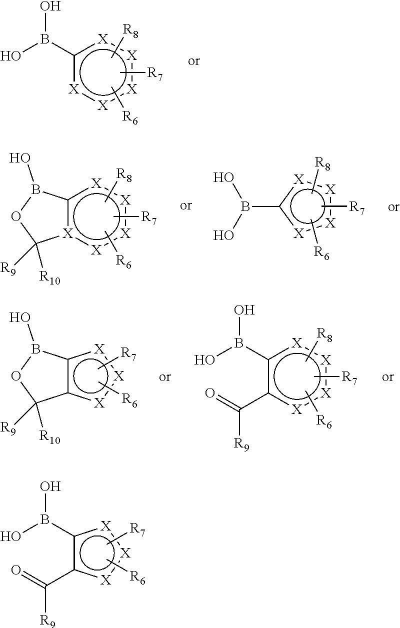

wherein
R₆ to R₈ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₉ and R₁₀ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₆ to R₈ are adjacent they may optionally be taken together to form one or more fused 5- or 6-membered aromatic, heteroaromatic, carbocyclic, or heterocyclic rings; and one of R₆ to R₈ comprises a bond to —C₂-TPB, or one of R₆ to R₈ comprises a bond to —C₁-E3ULB.

In accordance with the eleventh embodiment of the linkers of the present application, one of the linker elements L₁ or L₂ comprises the following structure, or salt, enantiomer, stereoisomer, or polymorph thereof:

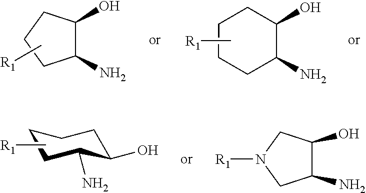

wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof:

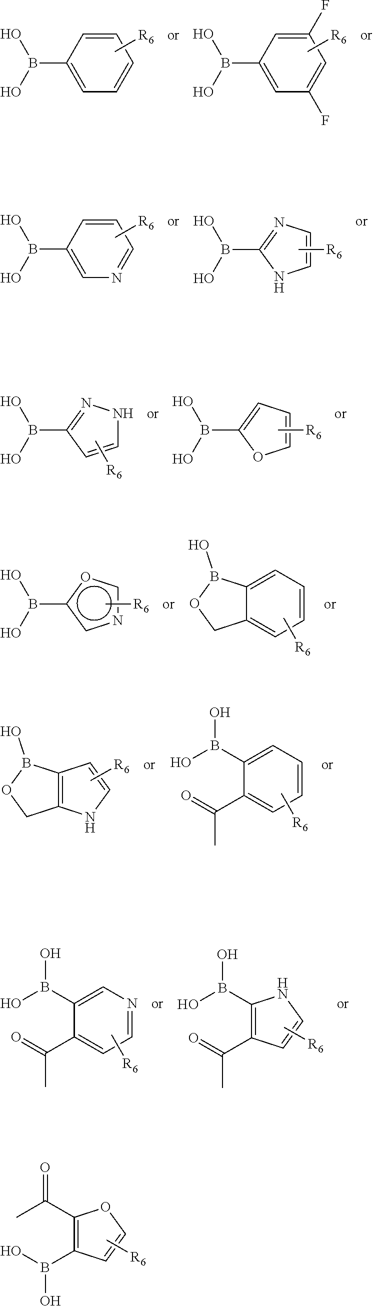

wherein
R₆ is a bond to either —C₁-E3ULB or —C₂-TPB.

In a twelfth embodiment of the linkers of the present application, one of the linker elements L₁ or L₂ is derived from a cis-1,3-aminoalcohol-containing compound comprising of the following structure, or salt, enantiomer, stereoisomer, or polymorph thereof:

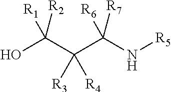

wherein
R₁ to R₄ and R₆ to R₇ are independently —H, —CH₂OH, —CH₂NH₂, —COOH, —CONH₂, —C₁₋₆ alkyl, —C₁₋₆ alkoxy, aryl, heteroaryl, or a bond to —C₁-E3ULB or —C₂-TPB;
R₅ is —H, —NH₂, —NHMe, —NMe₂, —CH₂COOH, —C₁₋₆ alkyl, —C₁₋₆ alkoxy, aryl, heteroaryl, or a bond to —C₁-E3ULB or —C₂-TPB;
   wherein R₁ or R₂ can optionally be connected to either R₃, R₄, R₅, R₆, or R₇ to make a ring, such that the amino and alcohol moieties are cis with respect to each other; R₃ or R₄ can optionally be connected to R₅, R₆, or R₇ to make a ring, such that the amino and alcohol moieties are cis with respect to each other; R₅ or R₆ can optionally be connected to R₇ to make a ring, such that the amino and alcohol moieties are cis with respect to each other; and wherein one of R₁ to R₇ comprises a bond to —C₁-E3ULB or —C₂-TPB;

and the other linker element, L₂ or L₁, respectively, is derived from an aromatic or heteroaromatic boronic acid- or boronic ester- or 1,2-boronic acid and carbonyl-containing compound comprising of one of the following structures, or salts, enantiomers, stereoisomers, or polymorphs thereof:

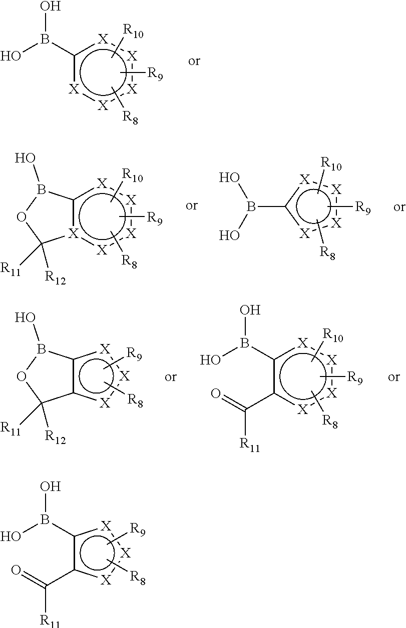

wherein
R₈ to R₁₀ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₁₁ and R₁₂ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
   wherein when two of R₈ to R₁₀ are adjacent they may optionally be taken together to form one or more fused 5- or 6-membered aromatic, heteroaromatic, carbocyclic, or heterocyclic rings; and one of R₈ to R₁₀ comprises a bond to —C₂-TPB, or one of R₈ to R₁₀ comprises a bond to —C₁-E3ULB.

In accordance with the twelfth embodiment of the of the present application, one of the linker elements L₁ or L₂ is comprised of one of the following structures, or salts, enantiomers, stereoisomers, or polymorphs thereof:

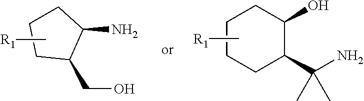

wherein
R₁ is a bond to either —C₁-E3ULB or —C₂-TPB.
and the other linker element, L₂ or L₁, respectively, is comprised of one of the following structures, or salts, enantiomers, stereoisomers, or polymorphs thereof:

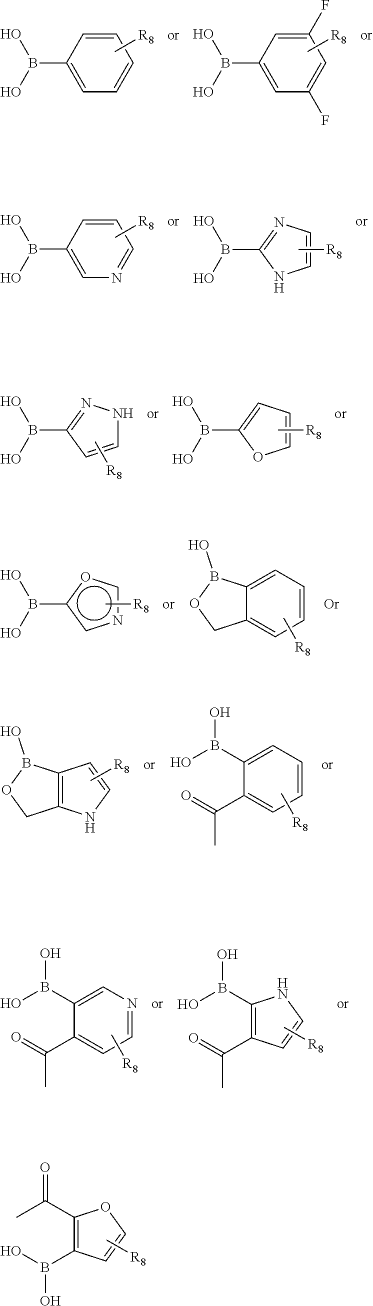

wherein
R₈ is a bond to either —C₁-E3ULB or —C-TPB.

In a thirteenth embodiment of the linkers of the therapeutic composition of the present application, one of the linker elements L₁ or L₂ is derived from an acyl or aromatic hydrazine-containing compound and is comprised of one of the following structures, or salts, enantiomers, stereoisomers, or polymorphs thereof:

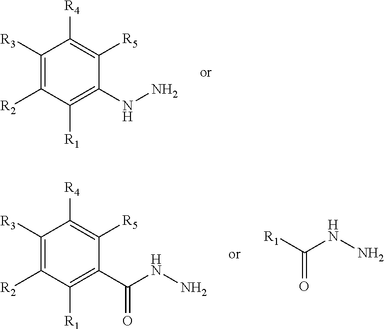

wherein
R₁ to R₅ are independently —H, —OH, —C₁₋₆ alkyl, —C₁₋₆ alkoxy, alkyl amine, aryl, heteroaryl, —C(O)NH₂, —CN, acyl, or a bond to —C₁-E3ULB or —C₂-TPB;
   wherein when two of R₁ to R₅ are adjacent they may optionally be taken together to form one or more fused 5- or 6-membered aromatic, heteroaromatic, carbocyclic, or heterocyclic rings; and wherein one of R₁ to R₅ comprises a bond to —C₁-E3ULB or —C₂-TPB;
and the other linker element, L₂ or L₁, respectively, is derived from an aromatic or heteroaromatic 1,2-boronic acid and carbonyl-containing compound is comprised of one of the following structures, or salts, enantiomers, stereoisomers, or polymorphs thereof:

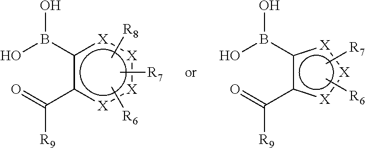

wherein
R₉ can be —C₁₋₆ alkyl, —C₁₋₆ alkoxy, aryl, heteroaryl, or a bond to —C₁-E3ULB or —C₂-TPB; and
R₆ to R₈ are independently —H, -halogen, —CF₃, —NO₂, —CN, —C₁₋₆ alkyl, —C₁₋₆ alkoxy, —C(O)CH₃, —C(O)CH₂CH₃,-acyl,-aryl, -heteroaryl, or a bond to —C₁-E3ULB or —C₂-TPB; and
X is independently C, N, O, or S; and
   wherein when two of R₆ to R₈ are adjacent they may optionally be taken together to form one or more fused 5- or 6-membered aromatic, heteroaromatic, carbocyclic, or heterocyclic rings; and wherein one of R₆ to R₈ comprises a bond to —C₂-TPB, or one of R₆ to R₈ comprises a bond to —C₁-E3ULB.

In accordance with the thirteenth embodiment of the linkers of the therapeutic composition of the present application, one of the linker elements L₁ or L₂ is comprised of one of the following structures, or salts, enantiomers, stereoisomers, or polymorphs thereof:

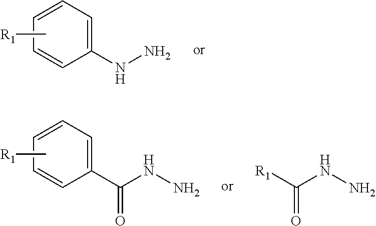

wherein
R₁ is a bond to —C₁-E3ULB or —C₂-TPB;
and the other linker element, L₂ or L₁, respectively, is comprised of one of the following structures, or salts, enantiomers, stereoisomers, or polymorphs thereof:

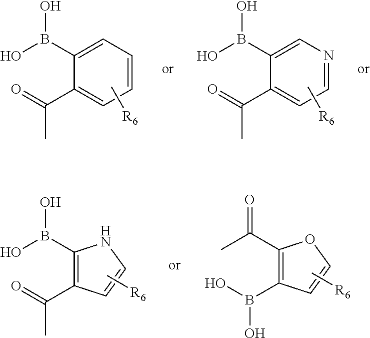

wherein
R₆ is a bond to either —C₁-E3ULB or —C₂-TPB.

In a fourteenth embodiment of the linkers of the present application, one of the linker elements L₁ or L₂ is derived from an α-hydroxyketone-containing compound and is comprised of one of the following structures, or salts, enantiomers, stereoisomers, or polymorphs thereof:

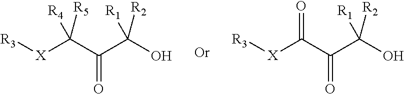

wherein
X is independently N or O; and
R₁ to R₅ are independently —H, —CH₃, -Ph, a bond to —C₁-E3ULB or —C₂-TPB, or can be connected to each other via a 3-, 4-, 5-, or 6-membered ring; and wherein one of R₁ to R₅ independently comprises a bond to —C₁-E3ULB or —C₂-TPB.

In accordance with the fourteenth embodiment of the linkers of the present application, one of the linker elements L₁ or L₂ is comprised of one of the following structures, or salts, enantiomers, stereoisomers, or polymorphs thereof:

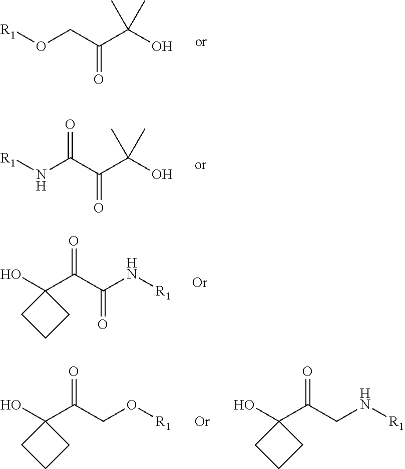

wherein
R₁ is a bond to —C₁-E3ULB or —C₂-TPB.

The above linker elements are suitable for assembly via two or more reversible covalent bonds that form under physiological conditions to generate therapeutically useful dimers in vivo to bring an E3 ligase or ligase machinery in close proximity to the BET domain target protein (i.e., BRD4).

In one embodiment, the therapeutic composition comprises a first precursor compound comprising an aromatic 1,2-diol-containing moiety of the linker element that is suitable for forming reversible covalent bonds with a second precursor compound comprising an aromatic or heteroaromatic boronic acid- or boronic ester-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising an aromatic 1,2-carbonyl and alcohol-containing moiety of the linker element that is suitable for forming reversible covalent bonds with a second precursor compound comprising an aromatic or heteroaromatic boronic acid- or boronic ester-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising a cis-dihydroxycoumarin-containing moiety of the linker element that is suitable for forming reversible covalent bonds with a second precursor compound comprising an aromatic or heteroaromatic boronic acid- or boronic ester-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising an α-hydroxycarboxylic acid-containing moiety of the linker element that is suitable for forming reversible covalent bonds with a second precursor compound comprising an aromatic or heteroaromatic boronic acid- or boronic ester-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising an aromatic 1,3-diol-containing moiety of the linker element that is suitable for forming reversible covalent bonds with a second precursor compound comprising an aromatic or heteroaromatic boronic acid- or boronic ester-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising an aromatic 2-(aminomethyl)phenol-containing moiety of the linker element that is suitable for forming reversible covalent bonds with a second precursor compound comprising an aromatic or heteroaromatic boronic acid- or boronic ester- or 1,2-boronic acid and carbonyl-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising a cis-1,2-diol or cis-1,3-diol-containing moiety of the linker element that is suitable for forming reversible covalent bonds with a second precursor compound comprising an aromatic or heteroaromatic boronic acid- or boronic ester-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising a [2.2.1] bicyclic ring system comprising a cis-1,2-diol-, or a cis-1,2-diol and cis-1,3-diol-, or a cis-1,2-diol and a β-hydroxyketone-containing moiety of the linker element that is suitable for forming reversible covalent bonds with a second precursor compound comprising an aromatic or heteroaromatic boronic acid- or boronic ester-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising a [2.2.1] bicyclic ring system comprising a cis-1,2-diol and amino or hydrazine-containing moiety of the linker element that is suitable for forming reversible covalent bonds with a second precursor compound comprising an aromatic or heteroaromatic boronic acid- or 1,2-boronic acid and carbonyl-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising a [2.2.1] bicyclic ring system comprising a cis-1,2-aminoalcohol and cis-1,3-diol- or a cis-1,2-aminoalcohol and a β-hydroxyketone-containing moiety of the linker element that is suitable for forming reversible covalent bonds with a second precursor compound comprising an aromatic or heteroaromatic boronic acid- or 1,2-boronic acid and carbonyl-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising a cis-1,2-aminoalcohol-containing moiety of the linker element that is suitable for forming reversible covalent bonds with a second precursor compound comprising an aromatic or heteroaromatic boronic acid- or boronic ester- or 1,2-boronic acid and carbonyl-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising a cis-1,3-aminoalcohol-containing moiety of the linker element that is suitable for forming reversible covalent bonds with a second precursor compound comprising an aromatic or heteroaromatic boronic acid- or boronic ester- or 1,2-boronic acid and carbonyl-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising an acyl or aromatic hydrazine containing moiety of the linker element that is suitable for forming reversible covalent bonds with a second precursor compound comprising an aromatic or heteroaromatic 1,2-boronic acid and carbonyl-containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

In another embodiment, the therapeutic composition comprises a first precursor compound comprising an α-hydroxyketone containing moiety of the linker element that is suitable for forming reversible covalent bonds with a second precursor compound comprising an α-hydroxyketone containing moiety of the linker element, wherein one compound independently comprises the E3 ligase or ligase machinery binding moiety bound to a connector, —C₁-E3ULB, and the other compound independently comprises the BET domain target protein binding moiety bound to a connector, —C₂-TPB.

Some of the above linker element families as well as additional reversible linker families are described in detail in U.S. Pat. Nos. 9,771,345; 8,853,185; and U.S. Pat. No. 9,943,603 to Barany et al., which are hereby incorporated by reference in their entirety.

### Connectors

Connectors are used to connect the linker element to the pharmacophore or ligand. The connector enables the correct spacing and geometry between the linker element and the pharmacophore such that the CURE-PRO dimer formed from the monomers orients the pharmacophores or ligands to allow high affinity binding of the pharmacophores or ligands to the protein target and the E3 ligase machinery during formation of the quaternary complex. The connector itself may function as a secondary pharmacophore by forming favorable interactions with the protein target and/or the E3 ligase machinery, which may enhance the direct interaction between the protein target and the E3 ligase machinery. The ideal connectors allow for modular assembly of CURE-PRO monomers through facile chemical reactions between reactive groups on the connector and complementary reactive groups on the linker elements and pharmacophores. Additionally, the portions of the embodiments below may be combined to form composite connector elements.

In a first embodiment of the connector element of the therapeutic composition of the present application, connector element C₁ and/or C₂ comprises the following structure, or salt, enantiomer, stereoisomer, or polymorph thereof:

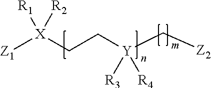

wherein
n and m are independently integers from 0 to 6;
X and Y are independently O, N, C, S, Si, P, or B;
R₁ to R₄ can independently be —H, —OH, —C₁₋₆ alkyl, —C₁₋₆ alkoxy, alkyl amine, aryl, heteroaryl, or —C(O)NH₂; and
Z₁ and Z₂ are independently a bond to -E3ULB, -TPB, -L₁ or -L₂; wherein when Z₁ is a bond to -E3ULB or -TPB, Z₂ is a bond to -L₁, or -L₂; and wherein when Z₁ is a bond to -L₁, or -L₂, Z₂ is a bond to -E3ULB or -TPB.

In accordance to the first embodiment of the connector element of the therapeutic composition of the present application, connector element C₁ and/or C₂ is comprised of one of the following structures, or salts, enantiomers, stereoisomers, or polymorphs thereof:

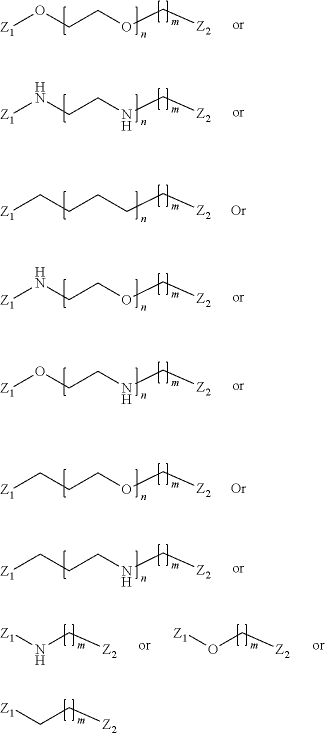

wherein
n and m are independently integers from 0 to 6; and
Z₁ and Z₂ are independently a bond to -E3ULB, -TPB, -L₁ or -L₂; wherein when Z₁ is a bond to E3ULB or -TPB, Z₂ is a bond to L₁, or -L₂; and wherein when Z₁ is a bond to -L₁, or -L₂, Z₂ is a bond to -E3ULB or -TPB.

In a second embodiment of the connector element of the therapeutic composition of the present application, connector element C₁ and/or C₂ comprises the following structure, or salt, enantiomer, stereoisomer, or polymorph thereof:

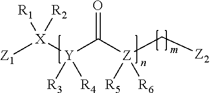

wherein
n and m are independently integers from 0 to 6;
X, Y, and Z are independently O, N, C, S, Si, P, or B; and
R₁ to R₆ are independently be —H, —OH, —C₁₋₆ alkyl, —C₁₋₆ alkoxy, alkyl amine, aryl, heteroaryl, or —C(O)NH₂;
   wherein R₃ to R₆ may optionally be fused to form 3-, 4-, 5-, 6-, 7-, or 8-membered cyclic or heterocyclic moieties; and
Z₁ and Z₂ are independently a bond to -E3ULB, -TPB, -L₁ or -L₂; wherein when Z₁ is a bond to -E3ULB or -TPB, Z₂ is a bond to -L₁ or -L₂; and wherein when Z₁ is a bond to -L₁ or -L₂, Z₂ is a bond to -E3ULB or -TPB.

In accordance with the second embodiment of the connector element of the therapeutic composition of the present application, connector element C₁ and/or C₂ is comprised of one of the following structures, or salts, enantiomers, stereoisomers, or polymorphs thereof:

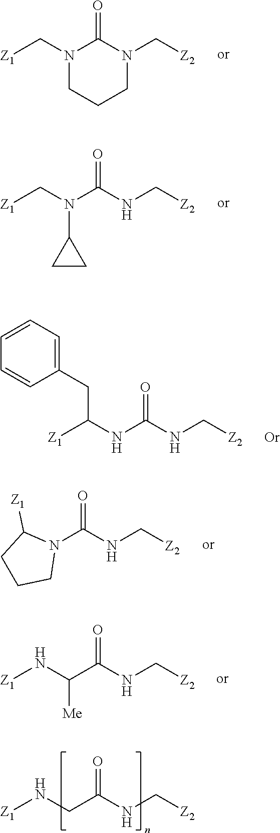

wherein
n is an integer from 0 to 6; and
Z₁ and Z₂ are independently a bond to -E3ULB, -TPB, -L₁ or -L₂;
   wherein when Z₁ is a bond to -E3ULB or -TPB, Z₂ is a bond to -L₁ or -L₂; and wherein when Z₁ is a bond to -L₁ or -L₂, Z₂ is a bond to -E3ULB or -TPB.

In a third embodiment of the connector element of the therapeutic composition of the present application, connector element C₁ and/or C₂ is comprised of one of the following structures, or salts, enantiomers, stereoisomers, or polymorphs thereof:

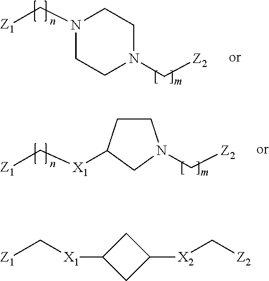

wherein
n and m are independently integers from 0-10; and
X₁ and X₂ are independently C, O, or N; and
Z₁ and Z₂ are independently a bond to -E3ULB, -TPB, -L₁ or -L₂;
wherein when Z₁ is a bond to -E3ULB or -TPB, Z₂ is a bond to -L₁ or -L₁; and wherein when Z₁ is a bond to -L₁ or -L₂, Z₂ is a bond to -E3ULB or -TPB.

In a fourth embodiment of the connector element of the therapeutic composition of the present application, connector element C₁ and/or C₂ is comprised of one of the following structures, or salts, enantiomers, stereoisomers, or polymorphs thereof:

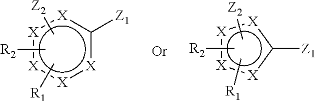

wherein
X is independently C, N, O, or S;
R₁ and R₂ can be independently —H, —OH, —C₁₋₆ alkyl, —C₁₋₆ alkoxy, alkyl amine, aryl, heteroaryl, or —C(O)NH₂, and
Z₁ and Z₂ are independently a bond to -E3ULB, -TPB, -L₁ or -L₂;
wherein when Z₁ is a bond to -E3ULB or -TPB, Z₂ is a bond to -L₁ or -L₂; and wherein when Z₁ is a bond to -L₁ or -L₂, Z₂ is a bond to -E3ULB or -TPB.

In a fifth embodiment of the connector element of the therapeutic composition of the present application, connector element C₁ and/or C₂ is comprised of one of the following structures, or salts, enantiomers, stereoisomers, or polymorphs thereof:

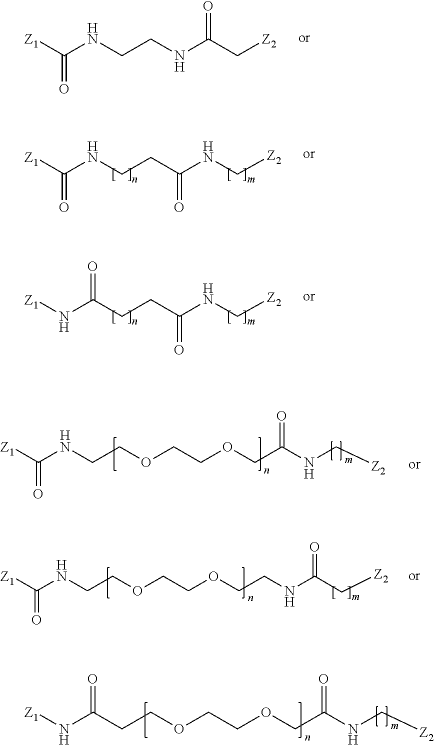

wherein
n and m are independently integers from 0-10; and
Z₁ and Z₂ are independently a bond to -E3ULB, -TPB, -L₁ or -L₂;
   wherein when Z₁ is a bond to -E3ULB or -TPB, Z₂ is a bond to -L₁ or -L₂; and wherein when Z₁ is a bond to -L₁ or -L₂, Z₂ is a bond to -E3ULB or -TPB.

In a sixth embodiment of the connector element of the therapeutic composition of the present application, connector element C₁ and/or C₂ is comprised of one of the following structures, or salts, enantiomers, stereoisomers, or polymorphs thereof:

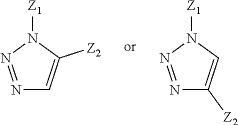

wherein
Z₁ and Z₂ are independently a bond to -E3ULB, -TPB, -L₁ or -L₂;
   wherein when Z₁ is a bond to -E3ULB or -TPB, Z₂ is a bond to -L₁ or -L₂; and wherein when Z₁ is a bond to L₁ or L₂, Z₂ is a bond to E3ULB or -TPB.

In a seventh embodiment of the connector element of the therapeutic composition of the present application, connector element C₁ and/or C₂ comprises the following structure, or salt, enantiomer, stereoisomer, or polymorph thereof:

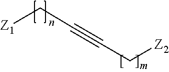

wherein
n and m are independently integers from 0-10; and
Z₁ and Z₂ are independently a bond to -E3ULB, -TPB, -L₁ or -L₂;
   wherein when Z₁ is a bond to E3ULB or -TPB, Z₂ is a bond to L₁ or -L₂; and wherein when Z₁ is a bond to -L₁ or -L₂, Z₂ is a bond to -E3ULB or -TPB.

In an eighth embodiment of the connector element of the therapeutic composition of the present application, the connector element C₁ and/or C₂ comprises the following structure, or salt, enantiomer, stereoisomer, or polymorph thereof:

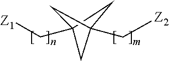

wherein
n and m are independently integers from 0-10; and
Z₁ and Z₂ are independently a bond to -E3ULB, -TPB, -L₁ or -L₂;
   wherein when Z₁ is a bond to -E3ULB or -TPB, Z₂ is a bond to -L₁ or -L₂; and wherein when Z₁ is a bond to -L₁ or -L₂, Z₂ is a bond to -E3ULB or -TPB.

### Pharmacophores or Ligands

Most drugs work by blocking protein activity, clogging an enzymatic pocket, and thus inhibiting activity. In order for a drug to bind, there needs to be sufficient complementarity and surface area of contact such that van der Waals, hydrogen bonding, and ionic interactions provide the requisite binding energy. The field of combinatorial chemistry is based on the principle of creating ligands or pharmacophores of different shapes and sizes, some of which can bind to the desired surface of the target, and thus serve as lead molecules for subsequent medicinal chemistry.

CURE-PROs have the advantage of being able to bind the target—E3 ligase macromolecular complex through two or more ligands or pharmacophores. These pharmacophores combine to give the CURE-PROs a tighter binding to the macromolecular complex than would be achieved through a single pharmacophore. Thus, even if one of the pharmacophores binds with poor affinity, i.e., dissociation constant around 10 μM, as long as the quaternary complex comprising: 1) the target protein, 2) the target-binding CURE-PRO, 3) the E3 ligase binding CURE-PRO, 4) the E3 ligase holds together long enough for the E2 enzyme to append ubiquitin(s) to the target protein, the CURE-PROs will work. In other words, the CURE-PRO drugs do not need to occupy an active site and inhibit activity to the 80-90% level (as required by traditional drugs), they just need to achieve an event (ubiquitination) to send the target protein to proteasomal destruction. In addition, CURE-PROs provide a linker element (and an optional connector), which may provide additional opportunities to maximize the surface area of interaction between the CURE-PRO and protein target—E3 ligase complex.

Pharmacophores may be moieties derived from molecules previously known to bind to target proteins, molecules that have been discovered to bind to target proteins after performing high-throughput screening of previously synthesized commercial or non-commercial combinatorial compound libraries, molecules that comprise of either natural or synthesized macrocycles, or molecules that are discovered to bind to target proteins by screening of newly synthesized combinatorial libraries. In contrast to traditional drugs, such pharmacophores do not need to inhibit activity, they just need to have affinity to the protein target.

Further, pharmacophores may be derived from traditional approaches such as fragment-based drug design and structure-based drug design. Those skilled in the art will recognize that any pharmacophore including pre-existing pharmacophores such as approved drugs are amenable to be designed as CURE-PROs through the incorporation of the appropriate linker elements and connectors. Previously approved drugs that have poor efficacy due to a low affinity for the protein target may still be utilized as a pharmacophore component of a CURE-PRO monomer. When such “poor binders” are combined with a second CURE-PRO monomer comprising a ligand that binds the E3 ligase, which in turn interacts with the protein target, the quaternary interactions result in overall enhanced binding and therefore higher efficacy.

The bromodomain and extra-terminal domain (BET) protein family includes BRD2, BRD3, BRD4 and the testis-specific BRDT (Segura et al., Cancer Res. 73:6264-6276 (2013), which is hereby incorporated by reference in its entirety). BET proteins are epigenetic readers that bind to acetylated histones at promoters and enhancers (Padmanabhan et al., J. Biosci. 41:295-311(2016), which is hereby incorporated by reference in its entirety) and subsequently activate RNA polymerase II-driven transcriptional elongation (Jang et al., Mol. Cell, 4:523-534 (2005), which is hereby incorporated by reference in its entirety) to play roles in the regulation of genes related to apoptosis and cell proliferation, including the proto-oncogenes MYC, Mcl1 and Bcl2 (Segura, M. F., Cancer Res. 73:6264-6276 (2013); Zong et al., Cancer Res. (2020), which are hereby incorporated by reference in their entirety). Recent work has shown the BRD4 BET domain protein plays a key role in driving MYC expression, and thus inhibition of BRD4 has been proposed to inhibit cancer progression (Zuber et al., Nature 478:524-528 (2011), which is hereby incorporated by reference in its entirety). BET domain protein binding moieties include JQ1 and OTX015, and suppress BET-dependent gene expression through the competitively displacement of the BRD proteins from the acetylated histones (Filippakopoulos et al., Nature 468: 1067-1073 (2010); Vizquez et al., Oncotarget 8: 7598-7613 (2017), which are hereby incorporated by reference in their entirety).

Several groups have developed PROTACS that destroy BRD4, using both CRBN and VHL ligands to recruit the E3 ligase (Lu et al., Chem. Biol. 18; 22(6):755-63 (2015); Tanaka et al., Nat. Chem. Biol. 12(12):1089-1096 (2016); Zengerle et al., ACS Chem. Biol. 10:1770-1777 (2015); Gadd et al., Nat Chem. Biol. 13: 514-521 (2017), which are hereby incorporated by reference in their entirety).

TPBs useful in the therapeutic composition of the present application target the following molecules: (1) G-protein coupled receptors; (2) nuclear receptors; (3) voltage gated ion channels; (4) ligand gated ion channels; (5) receptor tyrosine kinases; (6) growth factors; (7) proteases; (8) sequence specific proteases; (9) phosphatases; (10) protein kinases; (11) tumor suppressor genes; (12) cytokines; (13) chemokines; (14) viral proteins; (15) cell division proteins; (16) scaffold proteins; (17) DNA repair proteins; (18) ubiquitin ligases and ubiquitin complexes; (19) histone modifying enzymes; (20) apoptosis regulators; (21) chaperone proteins; (22) serine/threonine protein kinases: (23) cyclin dependent kinases; (24) growth factor receptors; (25) proteasome; (26) signaling protein complexes; (27) protein/nucleic acid transporters; (28) viral capsids; (29) viral proteins; (30) chromatin remodeling proteins; (31) extracellular matrix proteins; (32) cell adhesion proteins; (33) transmembrane proteins; (34) DNA modifying enzymes; (35) RNA modifying enzymes; (36) hormones; (37) transmembrane receptors; (38) intracellular receptors; (39) DNA binding proteins; (40) transcription factors; (41) oncogenes; (42) RNA binding proteins; (43) immune system proteins; and (44) multi-component protein complexes.

In one embodiment of a TPB useful in the therapeutic composition of the present application, the TPB is a BET domain protein binding moiety. The BET domain protein binding moiety can have the following structure, or salts, enantiomers, stereoisomers, or polymorphs thereof:

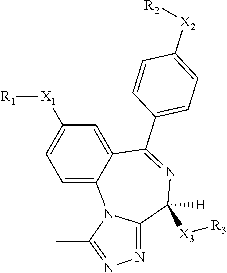

wherein
X₁ to X₃ are independently C, O, N, S, B, F, Cl, or Br;
R₁ to R₃ are independently a lone pair of electrons, —H, —C₁₋₆ alkyl, —C₁₋₆ alkoxy, alkyl amine, aryl, heteroaryl, —C₁₋₄ ester, —C(O)OH, an amide, or a bond to —C₂-L₂; and wherein one of R₁ to R₃ comprises a bond to —C₂-L₂.

In one embodiment, the TPB BET domain protein binding moiety has the structure:

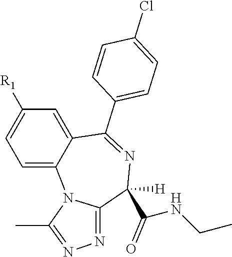

wherein R₁ comprises —C₂-L₂.

In a further embodiment, the BET domain protein binding moiety has the following structure, or salts, enantiomers, stereoisomers, or polymorphs thereof:

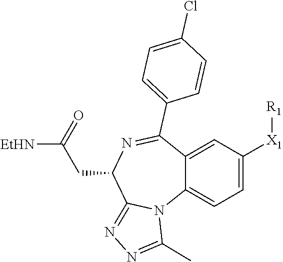

wherein
X₁ is C, O, N, S, or B; and
R₁ comprises a bond to —C₂-L₂;
and the E3ULB-C₁-L₁ first precursor compound is one of the following structures, or salts, enantiomers, stereoisomers, or polymorphs thereof:

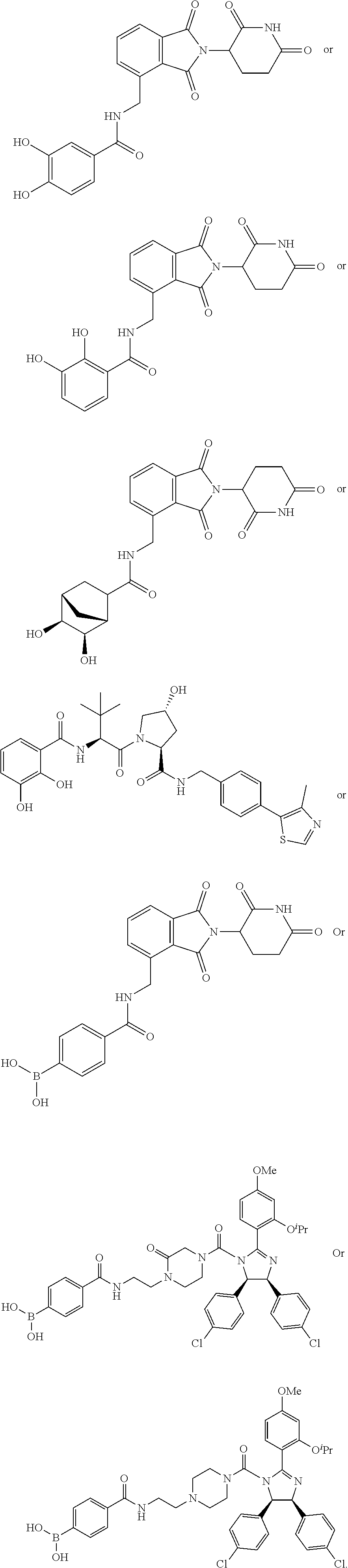

In another embodiment, the BET domain protein binding-containing second precursor compound is one of the following structures, or salts, enantiomers, stereoisomers, or polymorphs thereof:

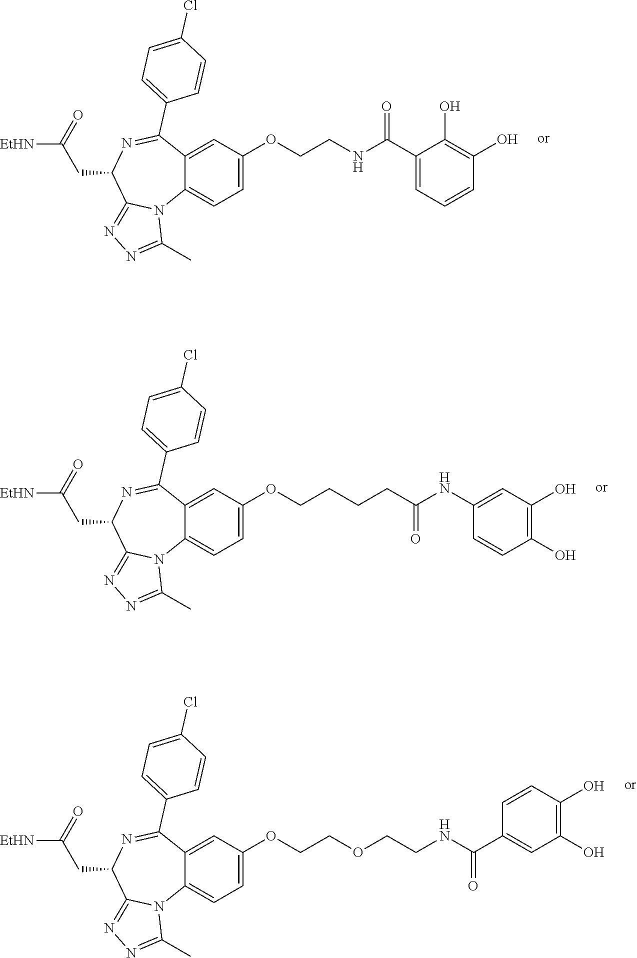

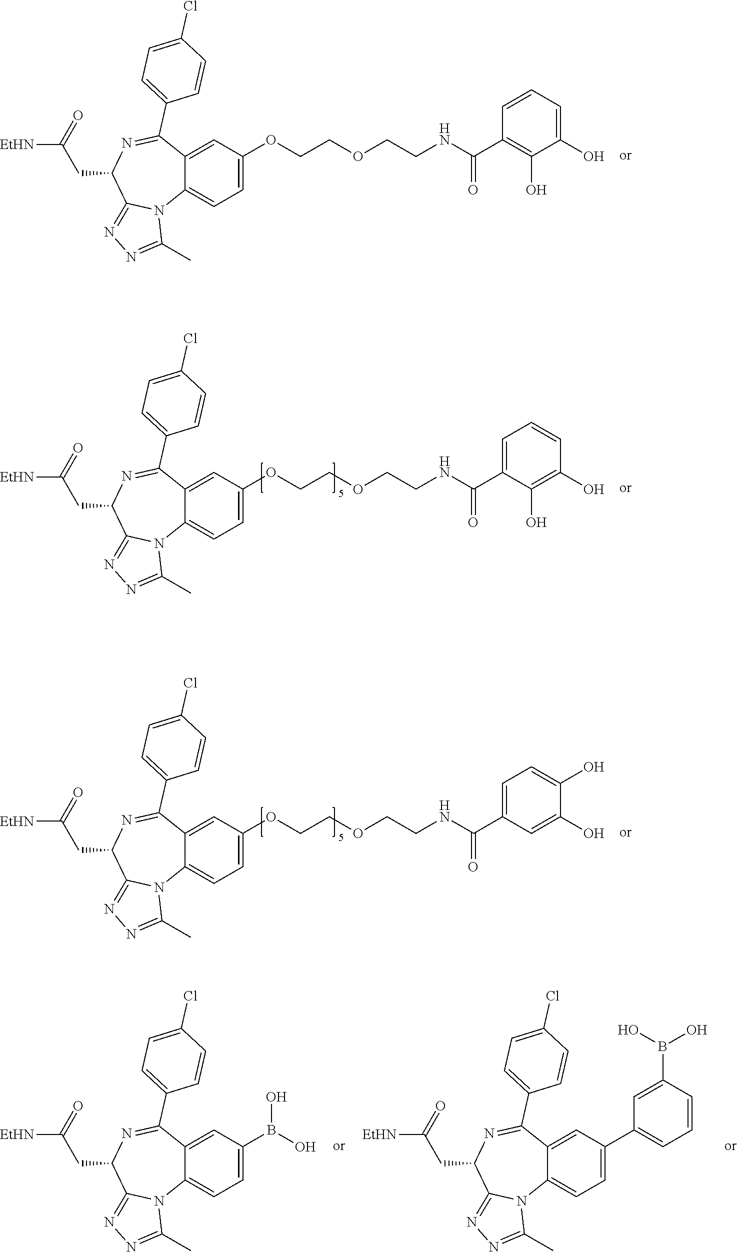

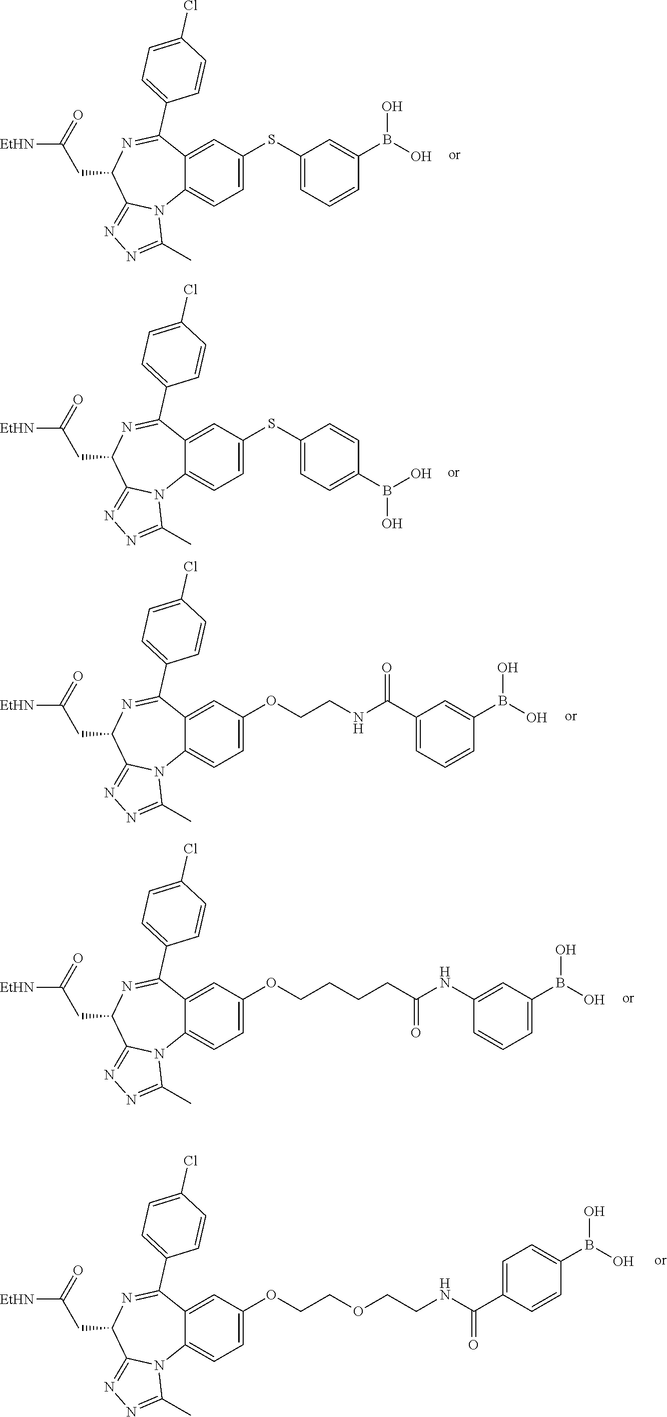

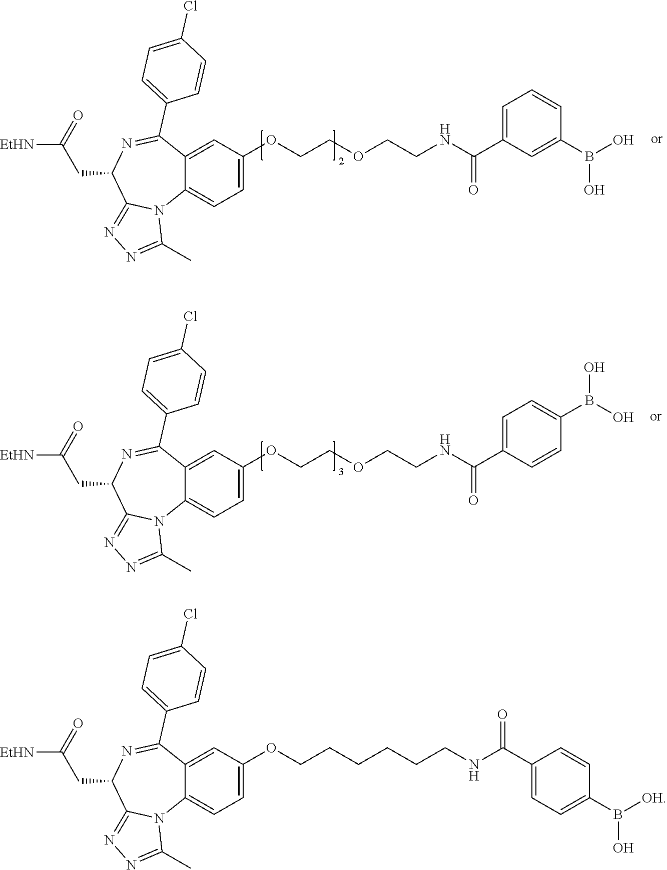

In a further embodiment, the BET domain protein binding moiety has the following structure, or salts, enantiomers, stereoisomers, or polymorphs thereof:

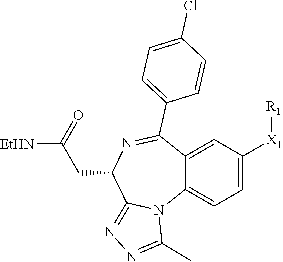

wherein
X₁ is C, O, N, S, or B;
R₁ comprises a bond to —C₂-L₂;
the linker element L₂ is comprised of an aromatic or heteroaromatic boronic acid- or boronic ester-containing moiety; and
the E3ULB-C₁-L₁ first precursor compound is one of the following structures, or salts, enantiomers, stereoisomers, or polymorphs thereof:

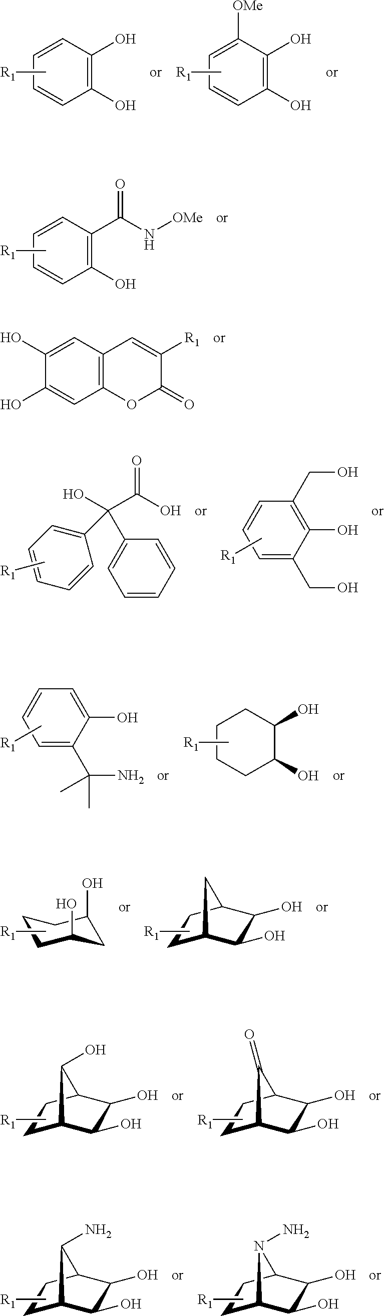

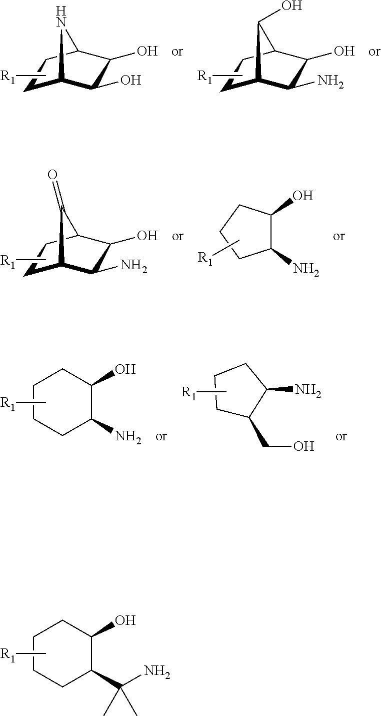

wherein
R₁ is —C₁-E3ULB.

In another embodiment, the BET domain protein binding-containing second precursor compound is one of the following structures, or salts, enantiomers, stereoisomers, or polymorphs thereof:

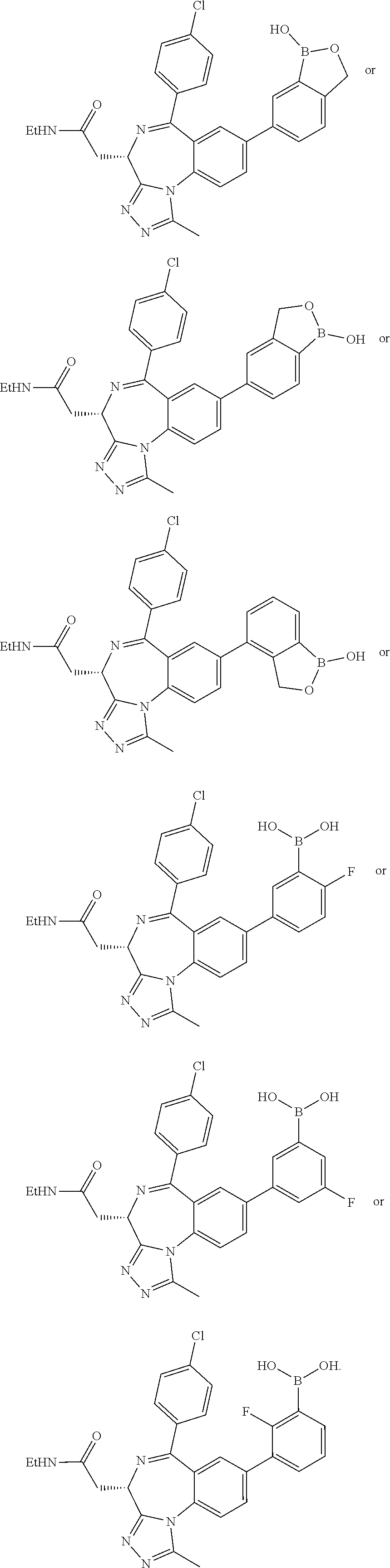

In yet another embodiment, the BET domain protein binding moiety has the following structure, or salts, enantiomers, stereoisomers, or polymorphs thereof:

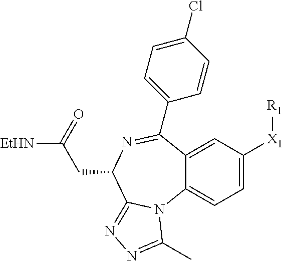

wherein
X₁ is C, O, N, S, or B; and
R₁ comprises a bond to —C₂-L₂;
the linker element L₂ is comprised of an aromatic or heteroaromatic boronic acid- or 1,2-boronic acid and carbonyl-containing moiety; and
the E3ULB-C₁-L₁ first precursor compound is one of the following structures, or salts, enantiomers, stereoisomers, or polymorphs thereof:

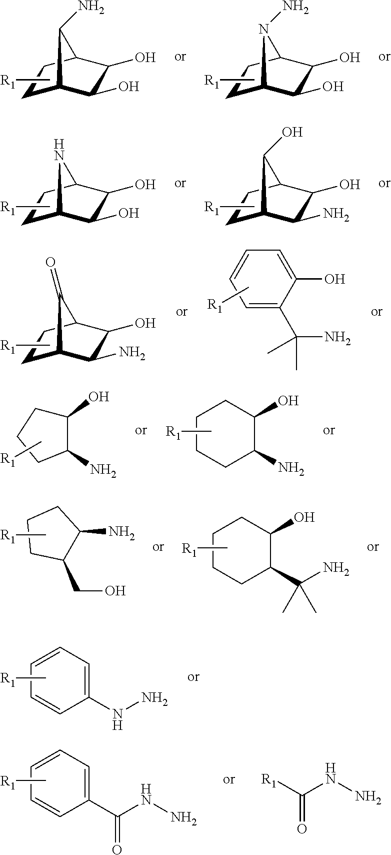

wherein
R₁ is —C₁-E3ULB.

In a further embodiment, the BET domain protein binding-containing second precursor compound is one of the following structures, or salts, enantiomers, stereoisomers, or polymorphs thereof:

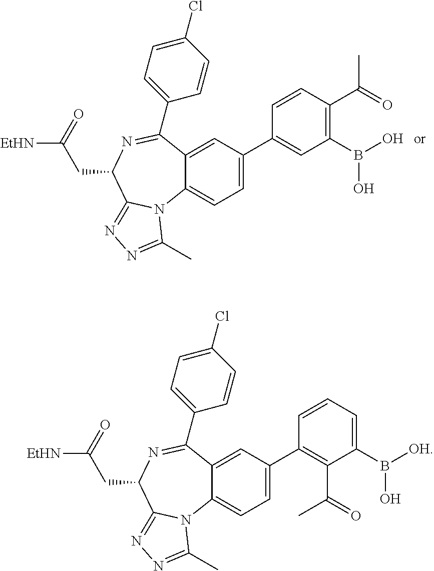

In another embodiment, the BET domain protein binding moiety has the following structure, or salts, enantiomers, stereoisomers, or polymorphs thereof:

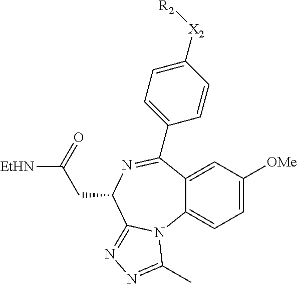

wherein
X₂ is C, O, N, S, or B; and
R₂ comprises a bond to —C₂-L₂;
   and the E3ULB-C₁-L₁-containing first precursor compound is one of the following structures, or salts, enantiomers, stereoisomers, or polymorphs thereof:

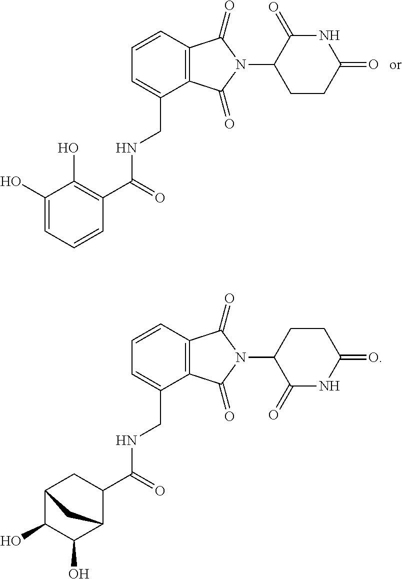 [SMILES: O=C1CCC(N2C(=O)c3cccc(CNC(=O)c4cccc(O)c4O)c3C2=O)C(=O)N1 ; O=C1CCC(N2C(=O)c3cccc(CNC(=O)C4C[C@@H]5C[C@H]4[C@@H](O)[C@H]5O)c3C2=O)C(=O)N1]

In yet another embodiment, the BET domain protein binding-containing second precursor compound has the following structure, or salts, enantiomers, stereoisomers, or polymorphs thereof:

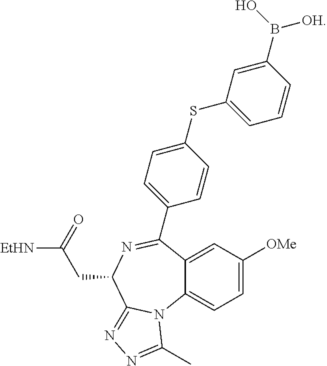

In a further embodiment, the BET domain protein binding has the following structure, or salts, enantiomers, stereoisomers, or polymorphs thereof:

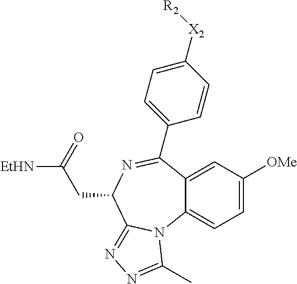

wherein
X₂ is C, O, N, S, or B; and
R₂ comprises a bond to —C₂-L₂;
the linker element L₂ is comprised of an aromatic or heteroaromatic boronic acid- or boronic ester-containing moiety; and
the E3ULB-C₁-L₁ first precursor compound is one of the following structures, or salts, enantiomers, stereoisomers, or polymorphs thereof:

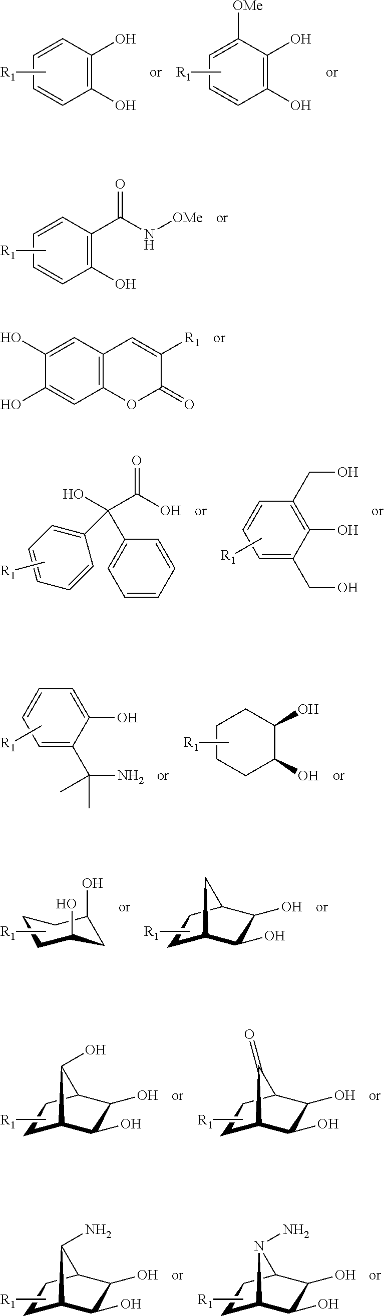

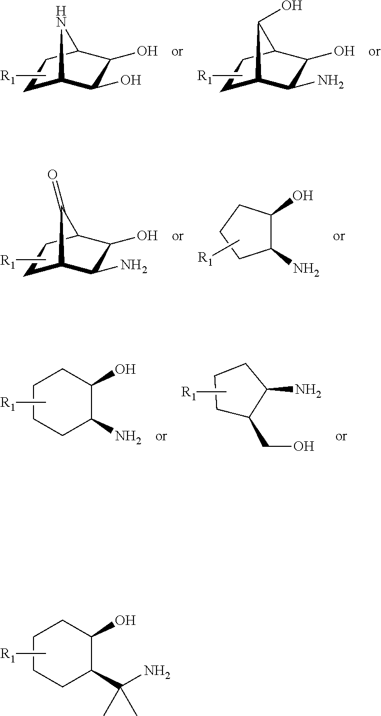

wherein
R₁ is —C₁-E3ULB.

In another embodiment, the BET domain protein binding moiety-containing second precursor compound is one of the following structures, or salts, enantiomers, stereoisomers, or polymorphs thereof:

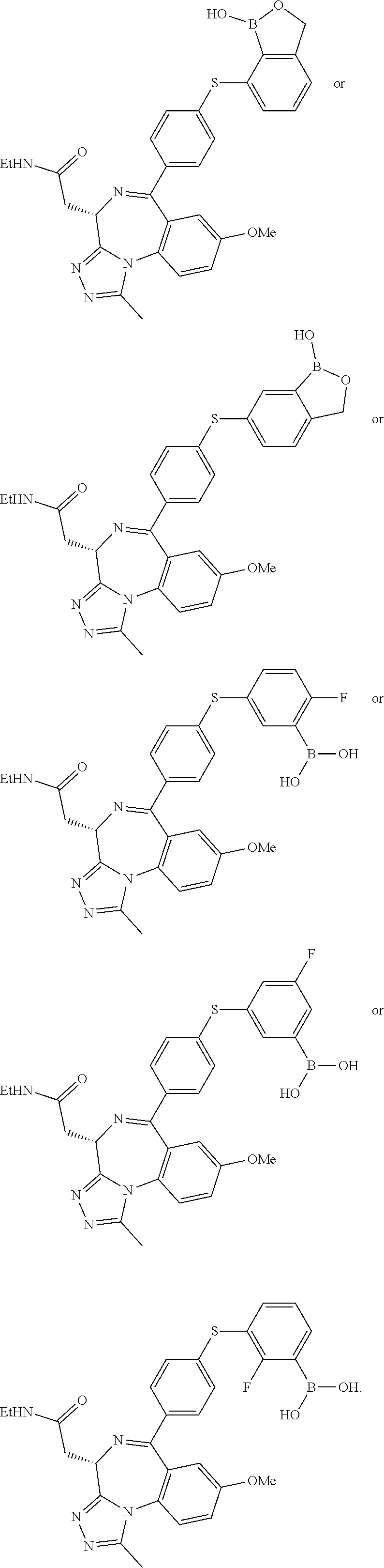

In yet another embodiment, the BET domain protein binding moiety has the following structure, or salts, enantiomers, stereoisomers, or polymorphs thereof:

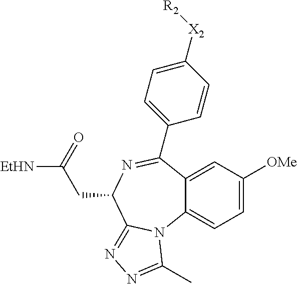

wherein
X₂ is C, O, N, S, or B; and
R₂ comprises a bond to —C₂-L₂;
the linker element L₂ is comprised of an aromatic or heteroaromatic boronic acid- or 1,2-boronic acid and carbonyl-containing moiety; and
the E3ULB-C₁-L₁ first precursor compound has one of the following structures, or salts, enantiomers, stereoisomers, or polymorphs thereof:

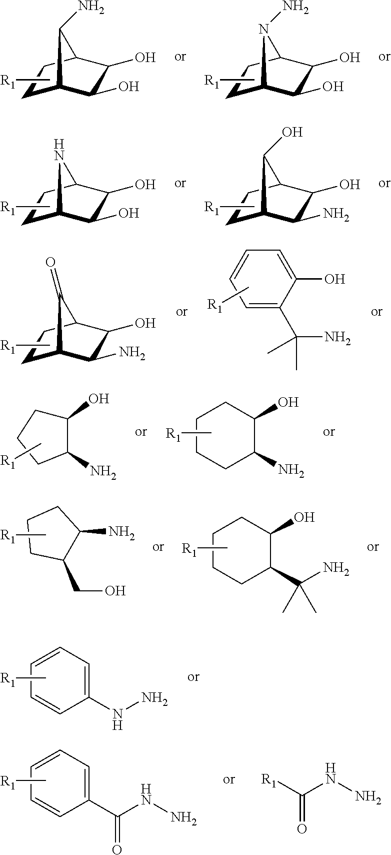

wherein
R₁ is-C₁-E3ULB.

In a further embodiment, the BET domain protein binding moiety-containing second precursor compound is one of the following structures, or salts, enantiomers, stereoisomers, or polymorphs thereof:

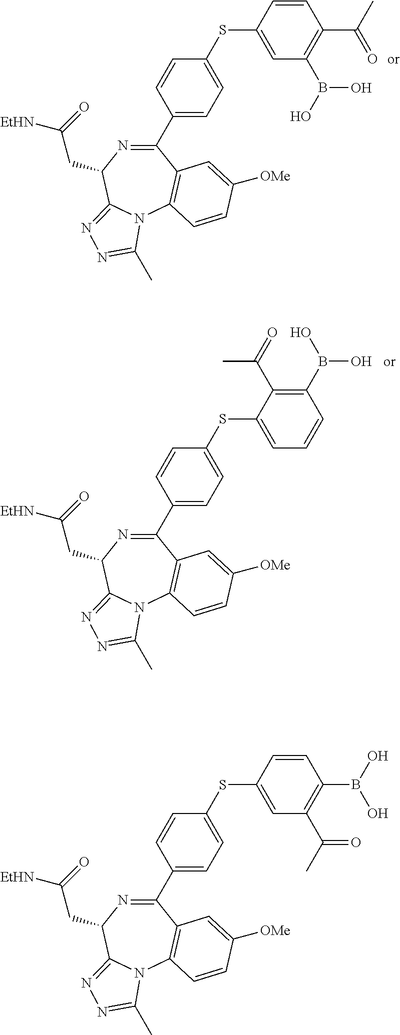

In another embodiment, the BET domain protein binding moiety has the following structure, or salts, enantiomers, stereoisomers, or polymorphs thereof:

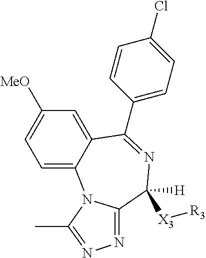

wherein
X₃ is C, O, N, S, or B; and
R₃ comprises a bond to —C₂-L₂;
the linker element L₂ is comprised of an aromatic or heteroaromatic boronic acid- or boronic ester-containing moiety; and
   and the E3ULB-C₁-L₁-containing first precursor molecule has one of the following structures, or salts, enantiomers, stereoisomers, or polymorphs thereof:

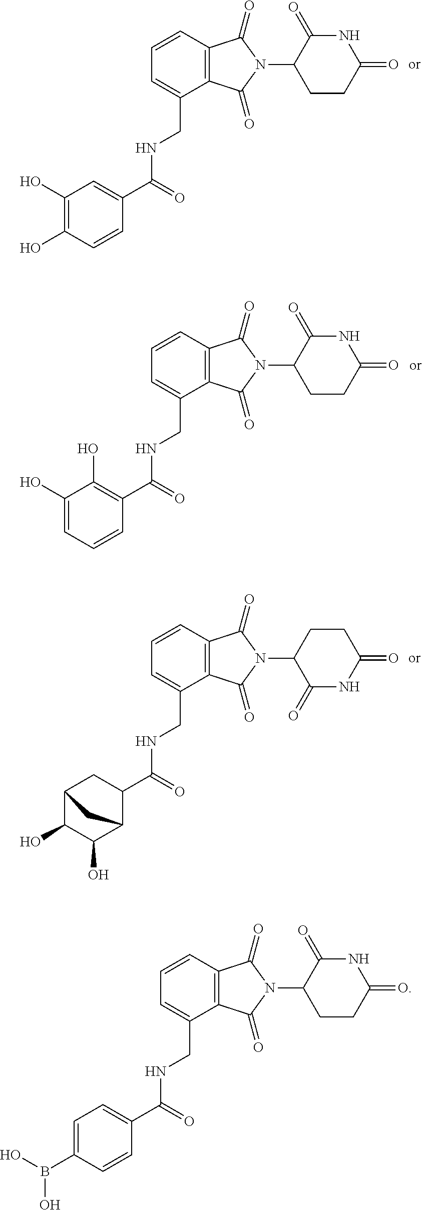 [SMILES: O=C1CCC(N2C(=O)c3cccc(CNC(=O)c4ccc(O)c(O)c4)c3C2=O)C(=O)N1 ; O=C1CCC(N2C(=O)c3cccc(CNC(=O)c4cccc(O)c4O)c3C2=O)C(=O)N1 ; O=C1CCC(N2C(=O)c3cccc(CNC(=O)C4C[C@@H]5C[C@H]4[C@@H](O)[C@H]5O)c3C2=O)C(=O)N1 ; O=C1CCC(N2C(=O)c3cccc(CNC(=O)c4ccc(B(O)O)cc4)c3C2=O)C(=O)N1]

In another embodiment, the TPB BET domain protein binding moiety has the structure:

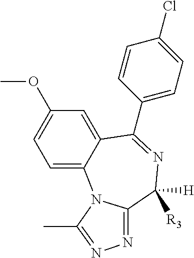

wherein R₃ comprises a bond to —C₂-L₂.

In yet another embodiment, the BET domain protein binding moiety-containing second precursor compound is one of the following structures, or salts, enantiomers, stereoisomers, or polymorphs thereof:

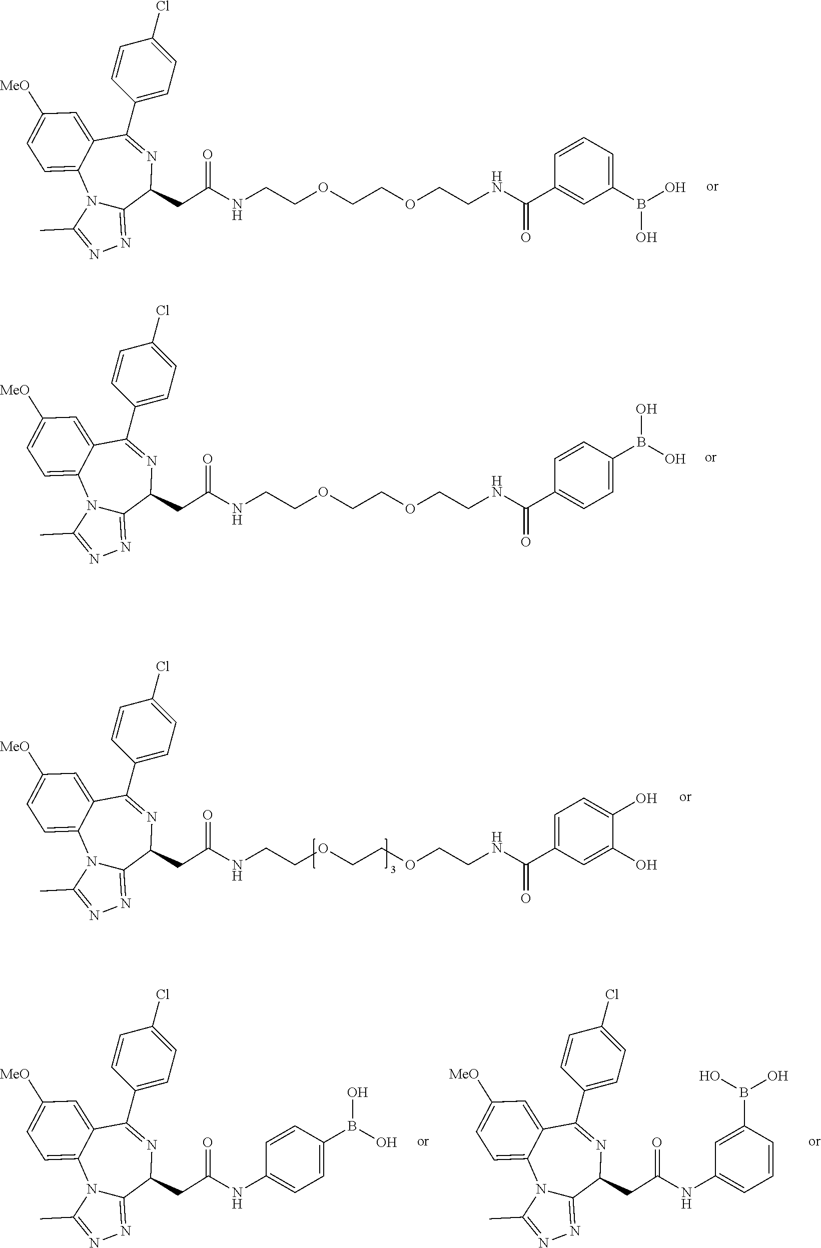

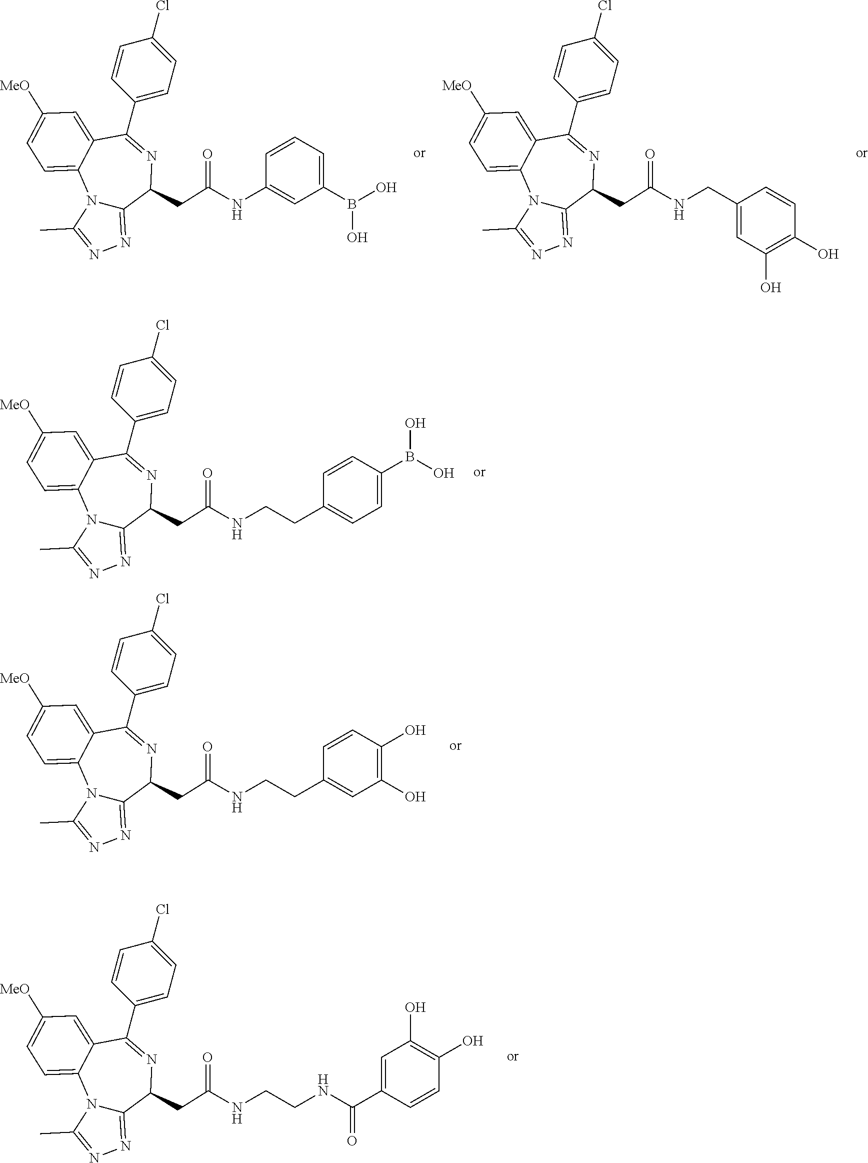

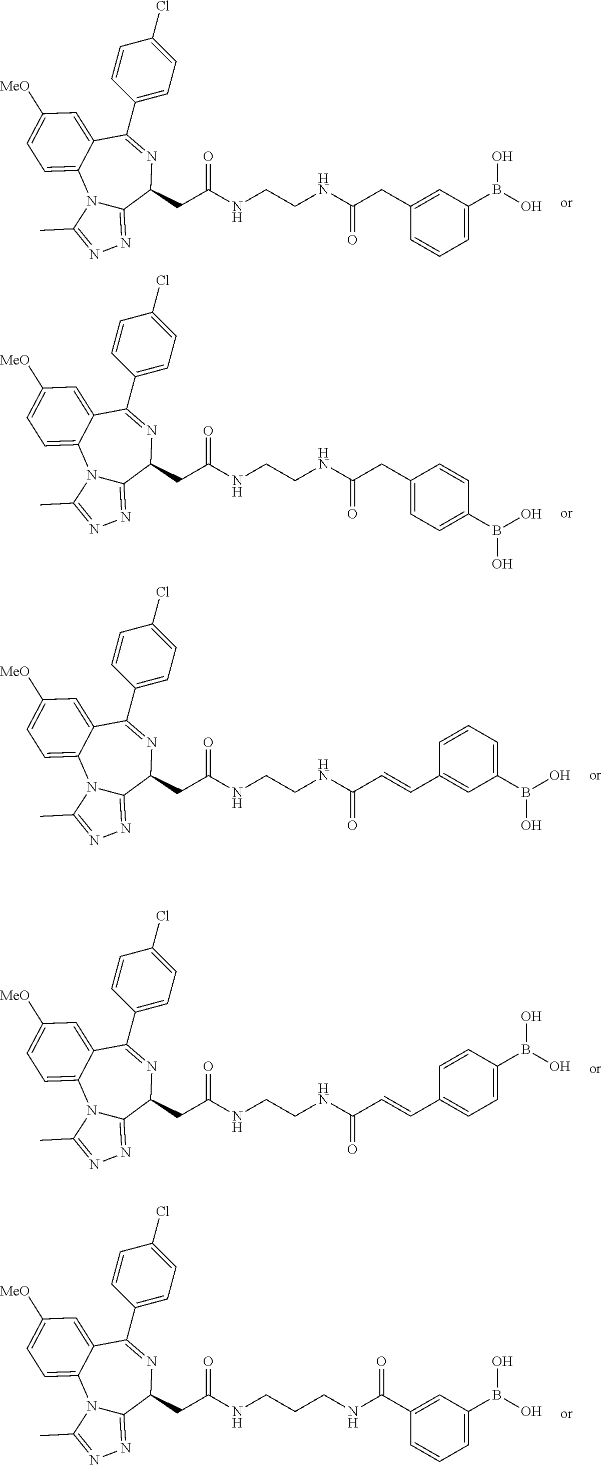 [SMILES: COc1ccc2c(c1)C(c1ccc(Cl)cc1)=N[C@@H](CC(=O)NCCNC(=O)Cc1cccc(B(O)O)c1)c1nnc(C)n1-2 ; COc1ccc2c(c1)C(c1ccc(Cl)cc1)=N[C@@H](CC(=O)NCCNC(=O)Cc1ccc(B(O)O)cc1)c1nnc(C)n1-2 ; COc1ccc2c(c1)C(c1ccc(Cl)cc1)=N[C@@H](CC(=O)NCCNC(=O)/C=C/c1cccc(B(O)O)c1)c1nnc(C)n1-2 ; COc1ccc2c(c1)C(c1ccc(Cl)cc1)=N[C@@H](CC(=O)NCCNC(=O)/C=C/c1ccc(B(O)O)cc1)c1nnc(C)n1-2 ; COc1ccc2c(c1)C(c1ccc(Cl)cc1)=N[C@@H](CC(=O)NCCCNC(=O)c1cccc(B(O)O)c1)c1nnc(C)n1-2]

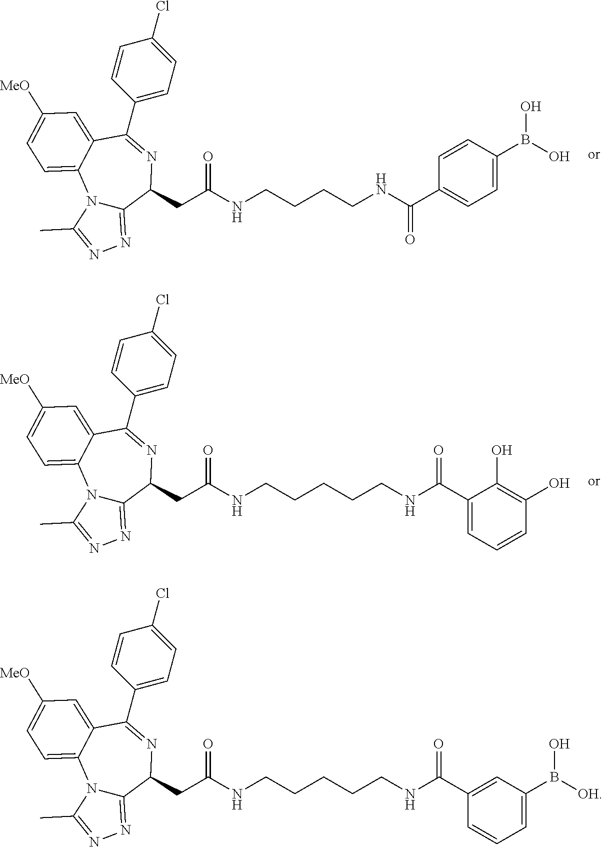 [SMILES: COc1ccc2c(c1)C(c1ccc(Cl)cc1)=N[C@@H](CC(=O)NCCCCNC(=O)c1ccc(B(O)O)cc1)c1nnc(C)n1-2 ; COc1ccc2c(c1)C(c1ccc(Cl)cc1)=N[C@@H](CC(=O)NCCCCCNC(=O)c1cccc(O)c1O)c1nnc(C)n1-2 ; COc1ccc2c(c1)C(c1ccc(Cl)cc1)=N[C@@H](CC(=O)NCCCCCNC(=O)c1cccc(B(O)O)c1)c1nnc(C)n1-2]

### CURE-PRO Molecules for Targeted Protein Degradation

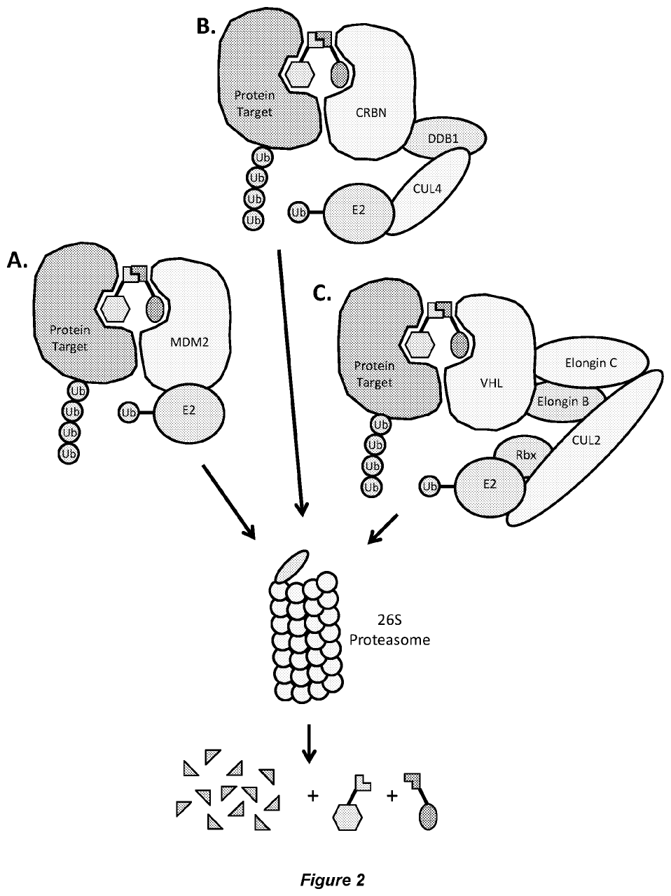

The regulation of cellular protein levels is achieved through control of their synthesis (i.e., transcriptional control), as well as control of their degradation. Intracellular degradation of proteins in eukaryotes is achieved by the ubiquitin-proteasome system, wherein motifs within proteins (known as degrons) are recognized by the E3 ubiquitin ligase machinery, which then marks the target proteins with ubiquitin to designate them for destruction (Mészáros, et al., Sci. Signal. 10(470) (2017), which is hereby incorporated by reference in its entirety). CURE-PRO molecules may be designed to exploit different ubiquitin-proteasome degradation pathways, as illustrated in FIG. 2. In one embodiment of the present application, HECT-type E3 ligases (i.e., HERC or NEDD4 family), RING-between-RING E3 ligases (i.e., MDM2, CBL), or other RING domain variants (i.e., TRIM subfamily) may be recruited by a suitable CURE-PRO ligand and CURE-PRO pharmacophore to bind a desired protein target forming a complex that facilitates transfer of ubiquitin from E2 to the E3 ligase and then to the target (see FIG. 2, part A). Alternatively, Cullin-RING E3 ligase complexes (i.e., CULLIN2-Elongin B-Elongin C-VHL complex, or CULLIN4-DDB1-CRBN complex) may be recruited by a suitable CURE-PRO ligand (binding the substrate receptor subunit, i.e., VHL or CRBN) and CURE-PRO pharmacophore to bind a desired protein target forming a complex that facilitates transfer of ubiquitin from E2 directly to the target (see FIG. 2, part B and C). Alternatively, in some cases a chaperonin (i.e., HSP70) may be recruited by a suitable CURE-PRO ligand (i.e., comprising a hydrophobic surface that binds to HSP70) and CURE-PRO pharmacophore to bind a desired protein target forming a complex wherein an E3 ligase complex is recruited to HSP70 that facilitates transfer of ubiquitin from E2 to the target. In all the above examples, the resultant poly-ubiquitinated protein target is degraded by the 26S proteasome, releasing the two CURE-PRO monomers, which may then be recycled to facilitate catalytic degradation of additional molecules of the same protein targets, analogous to the PROTAC drugs (Bondeson and Crews, Annu. Rev. Pharmacol. Toxicol. 57:107-123(2017); Ottis and Crews, ACS Chem. Biol. 12(4):892-898 (2017); Lai and Crews, Nat. Rev. Drug Discov. 16(2):101-114 (2017), which are hereby incorporated by reference in their entirety). Note that FIG. 2, as well as subsequent figures, the proteins are not drawn to scale, relative to the CURE-PRO molecules, or other proteins, that other configurations that accomplish the same goal of facilitated target degradations are also envisioned, that the designation of “E3 Ligase” also encompasses E3 ligase complexes (e.g., FIG. 2 parts B and C), and that the E2 ubiquitination enzyme may append the ubiquitin either directly to the target(s) or indirectly through E3, and then to the target.

The success of the CURE-PRO approach relies on the combination of four interactions working simultaneously to create a quaternary structure: “Interaction A”—The reversible covalent link between the target binding CURE-PRO monomer and the E3 ligase binding CURE-PRO monomer; “Interaction B”—The affinity of the target-binding CURE-PRO monomer pharmacophore to the target; “Interaction C”—The affinity of the E3 ligase (machinery) binding CURE-PRO monomer ligand to the E3 ligase (machinery), and last but not least; “Interaction D”—The E3 ligase (machinery) interaction with the target. Manipulating any one of these four interactions may profoundly alter the selectivity, specificity, rate, or efficacy of CURE-PRO mediated target destruction.

It is estimated that there are over 600 E3 ubiquitin ligases encoded within the human genome, with only a small subset of these having a known substrate sequence, and even fewer with a known small molecule that binds to the substrate recognition pocket (Mesziros et al., Sci Signal. 10(470), (2107); Cromm and Crews, Cell Chem. Biol. 24(9):1181-1190, (2017); Schapira et al., Nat Rev Drug Discov. 18(12):949-963 (2019), which are hereby incorporated by reference in their entirety). Nevertheless, there are several known E3 ubiquitin ligase pharmacophores or ligands that bind to an E3 ligase or complex which are suitable for use in the CURE-PRO design.

A first embodiment of an E3 ubiquitin ligase pharmacophore or ligand that binds to the CRBN subunit of the CULLIN4A or CULLIN4B E3 ligase machinery are derived from thalidomide. These imide-based moieties have been widely used within the PROTAC field (Chan et al., J Med. Chem. 61(2): 504-513 (2017), which is hereby incorporated by reference in its entirety).

In one embodiment of the therapeutic composition of the present application, the E3ULB ubiquitin binding moiety binds to the CRBN subunit of the CULLIN4A or CULLIN4B E3 ligase machinery.

A generic structure of a CRBN ligand suitable for CURE-PRO degradation has the following structure, or salts, enantiomers, stereoisomers, or polymorphs thereof:

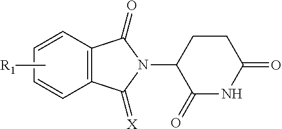

wherein
X is H₂, NH, O, or S; and
R₁ comprises a bond to —C₁-L₁.

In certain embodiments, the imide-based moiety is related to either pomalidomide or lenalidomide or has the following structure, or salts, enantiomers, stereoisomers, or polymorphs thereof:

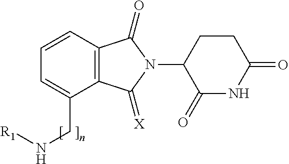

wherein
X is —H₂, —NH, —O, or —S;
n is an integer from 0-10; and
R₁ comprises a bond to —C₁-L₁;

A second generic structure of a CRBN ligand suitable for CURE-PRO degradation has the following structure, or salts, enantiomers, stereoisomers, or polymorphs thereof:

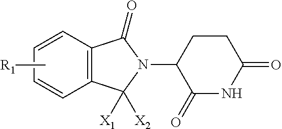

wherein
X₁ and X₂ are independently —H or —C₁₋₆ alkyl; and
R₁ comprises a bond to —C₁-L₁.

In certain embodiments, similar to CRBN ligands developed by Scheepstra et al., (Scheepstra et al., Comput. Struct. Biotechnol. J. 17:160-176 (2019), which is hereby incorporated by reference in its entirety), the imide-based moiety has the following structure, or salts, enantiomers, stereoisomers, or polymorphs thereof:

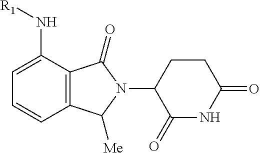

wherein
R₁ comprises —C₁-L₁.

A third generic structure of a CRBN ligand suitable for CURE-PRO degradation has the following structure, or salts, enantiomers, stereoisomers, or polymorphs thereof:

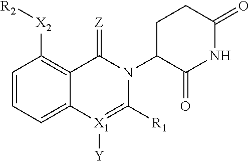

wherein
X₁ and X₂ are independently be C, O, N, or S;
R₁ or R₂ are independently —H; —C₁₋₆ alkyl, —C₁₋₆ alkoxy, alkyl amine, —C(O)NH₂, or a bond to —C₁-L₁;
Y is a lone pair, —H, —C₁₋₆ alkyl, —C₁₋₆ alkoxy, alkyl amine, —C(O)NH₂, or a bond to C₁-L₁; and
Z is —H₂, —NH, —O, or —S; and
wherein one of R₁, R₂, or Y comprises a bond to —C₁-L₁.

In certain embodiments, similar to CRBN ligands developed by Chamberlain & Cathers (Chamberlain & Cathers, Drug Discov. Today: Tech. 31: 29-34 (2019), which is hereby incorporated by reference in its entirety), the imide-based moiety has the following structure, or salts, enantiomers, stereoisomers, or polymorphs thereof:

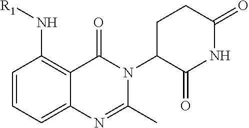

wherein R₁ comprises —C₁-L₁.

In a further embodiment, the E3ULB ubiquitin binding moiety that binds to the CRBN subunit of the CULLIN4A or CULLIN4B E3 ligase machinery is one of the following structures, or salts, enantiomers, stereoisomers, or polymorphs thereof:

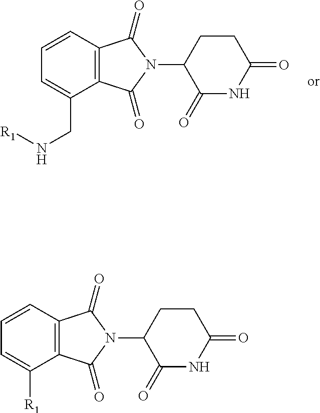

wherein
R₁ comprises a bond to —C₁-L₁.

In another embodiment, the E3ULB ubiquitin binding moiety that binds to the CRBN subunit of the CULLIN4A or CULLIN4B E3 ligase machinery is one of the following structures, or salts, enantiomers, stereoisomers, or polymorphs thereof:

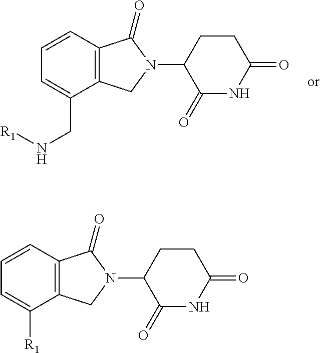

wherein
R₁ comprises a bond to —C₁-L₁.

In another embodiment of the therapeutic composition of compounds, the E3ULB-C₁-L₁-containing first precursor compound has the following structure, or salts, enantiomers, stereoisomers, or polymorphs thereof:

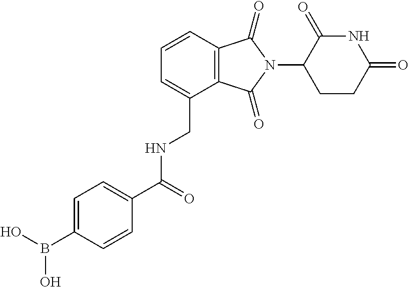 [SMILES: O=C1CCC(N2C(=O)c3cccc(CNC(=O)c4ccc(B(O)O)cc4)c3C2=O)C(=O)N1]

and the TPB-C₂-L₂ moiety-containing second precursor compound is one of the following structures, or salts, enantiomers, stereoisomers, or polymorphs thereof:

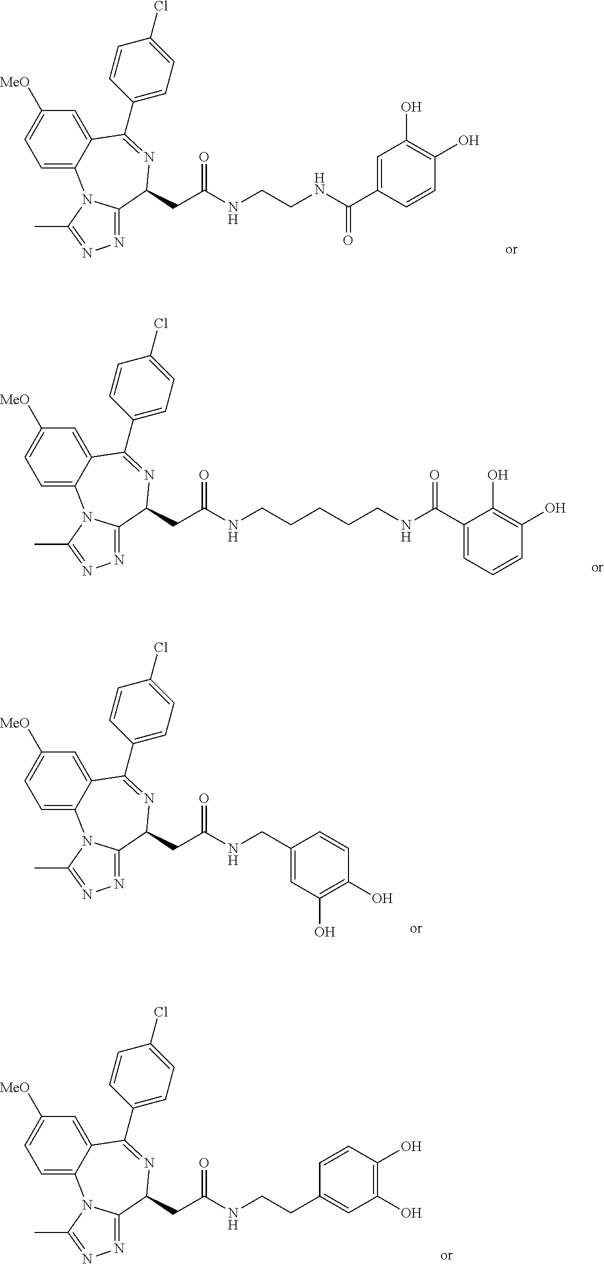 [SMILES: COc1ccc2c(c1)C(c1ccc(Cl)cc1)=N[C@@H](CC(=O)NCCNC(=O)c1ccc(O)c(O)c1)c1nnc(C)n1-2 ; COc1ccc2c(c1)C(c1ccc(Cl)cc1)=N[C@@H](CC(=O)NCCCCCNC(=O)c1cccc(O)c1O)c1nnc(C)n1-2 ; COc1ccc2c(c1)C(c1ccc(Cl)cc1)=N[C@@H](CC(=O)NCc1ccc(O)c(O)c1)c1nnc(C)n1-2 ; COc1ccc2c(c1)C(c1ccc(Cl)cc1)=N[C@@H](CC(=O)NCCc1ccc(O)c(O)c1)c1nnc(C)n1-2]

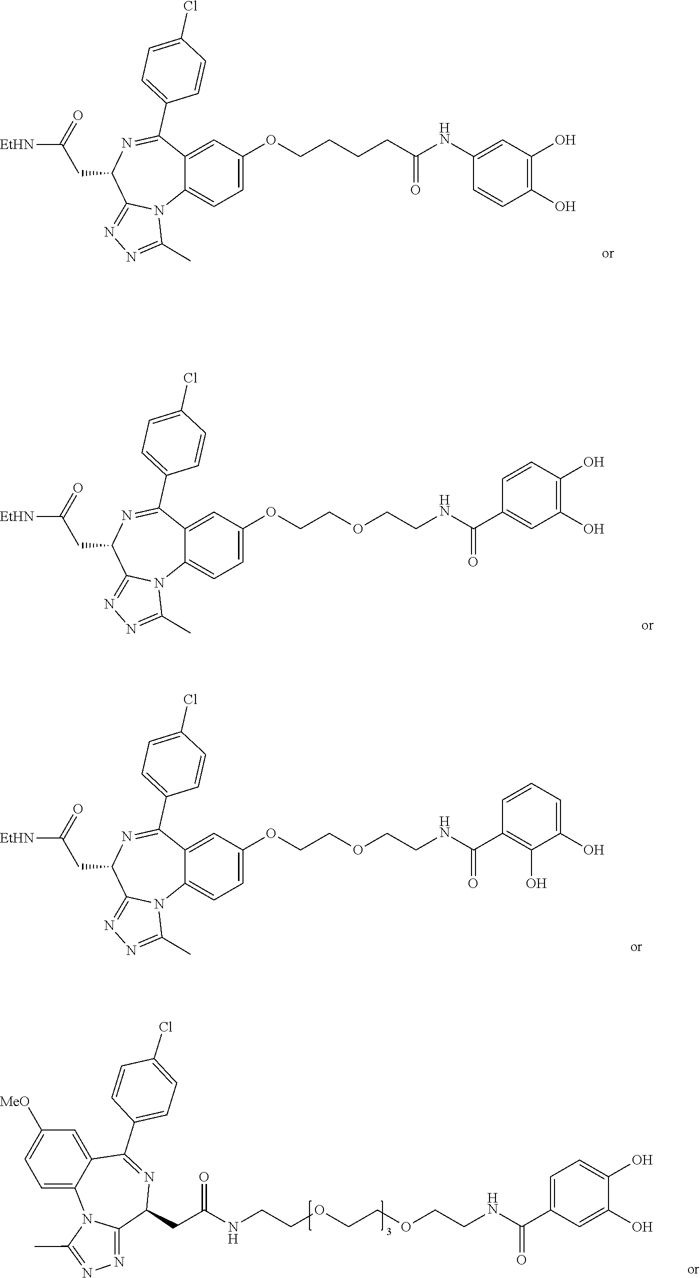

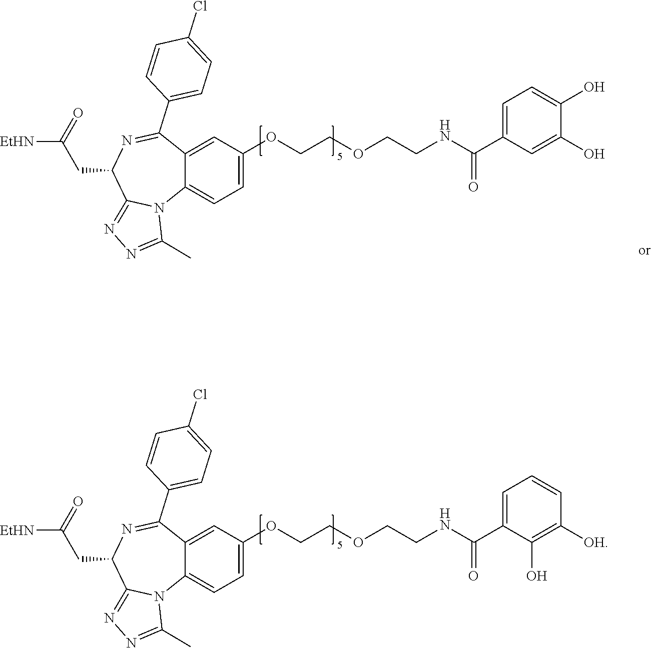

In a further embodiment of the therapeutic composition of compounds, the E3ULB-C₁-L₁ moiety-containing first precursor compound has the following structure, or salts, enantiomers, stereoisomers, or polymorphs thereof:

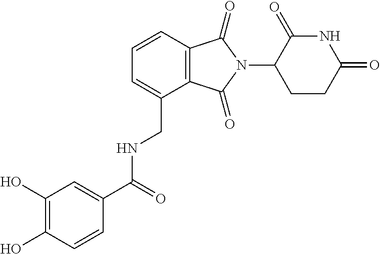 [SMILES: O=C1CCC(N2C(=O)c3cccc(CNC(=O)c4ccc(O)c(O)c4)c3C2=O)C(=O)N1]

and the TPB-C₂-L₂ moiety-containing second precursor compound is one of the following structures, or salts, enantiomers, stereoisomers, or polymorphs thereof:

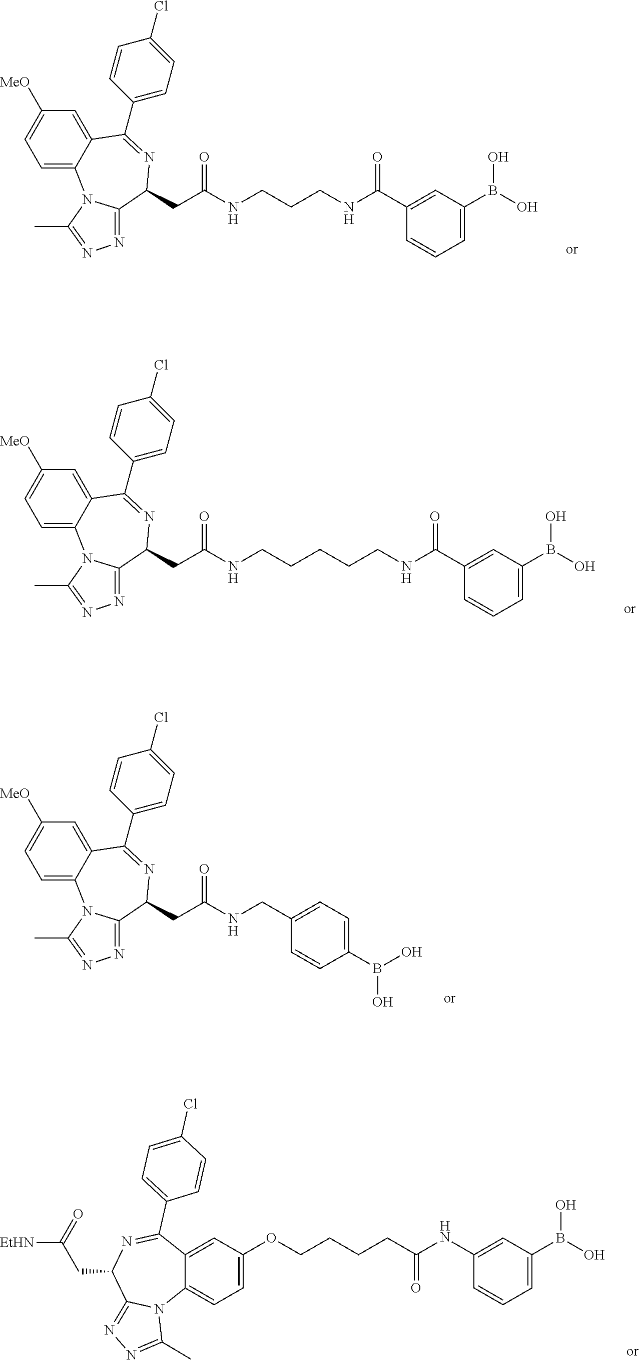

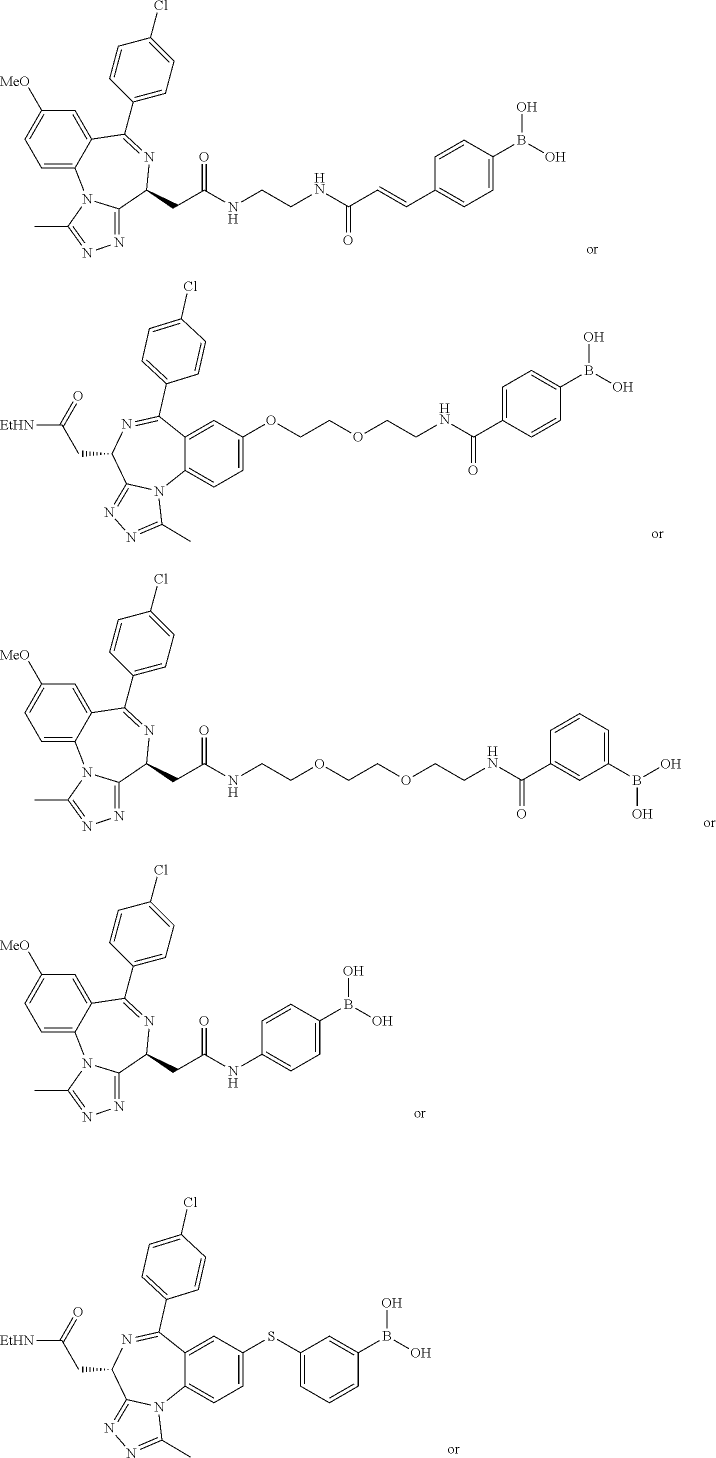

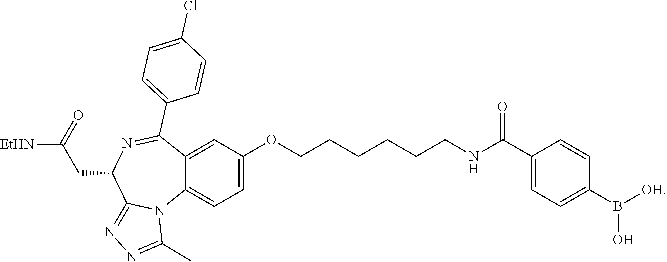

In yet another embodiment of the therapeutic composition of compounds, the E3ULB-C₁-L₁ moiety-containing first precursor compound has the following structure, or salts, enantiomers, stereoisomers, or polymorphs thereof:

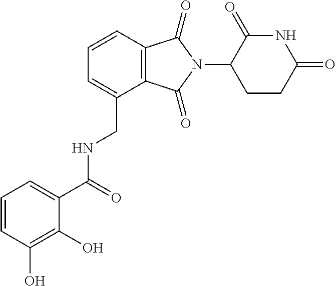 [SMILES: O=C1CCC(N2C(=O)c3cccc(CNC(=O)c4cccc(O)c4O)c3C2=O)C(=O)N1]

and the TPB-C₂-L₂ moiety-containing second precursor compound is one of the following structures, or salts, enantiomers, stereoisomers, or polymorphs thereof:

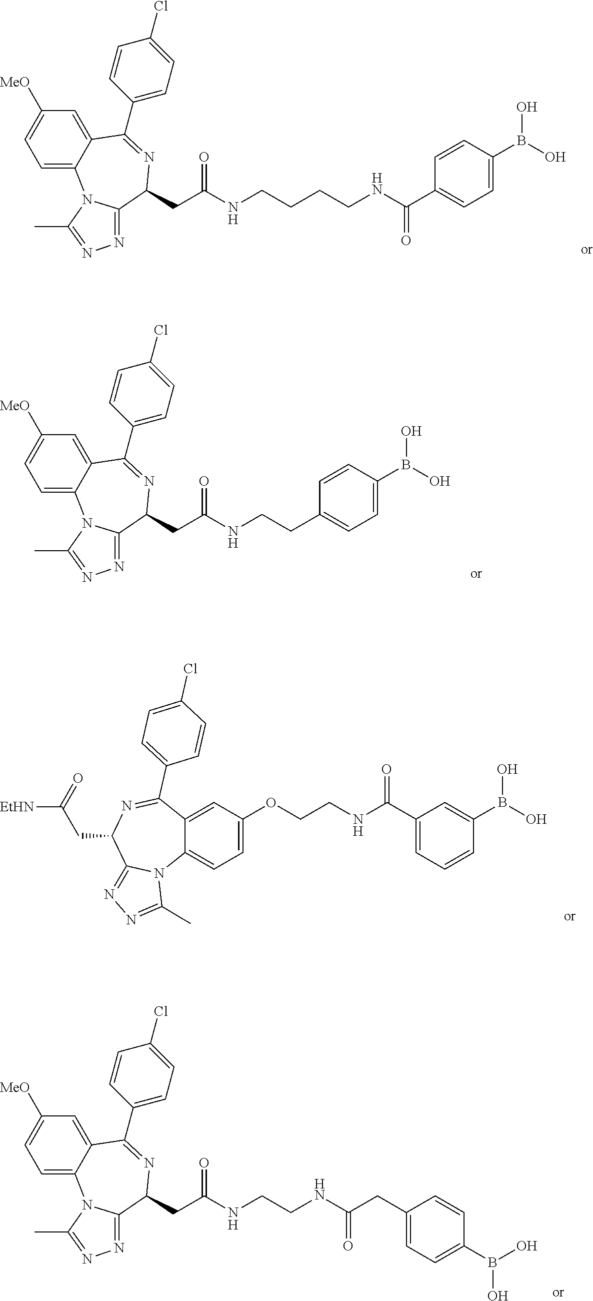

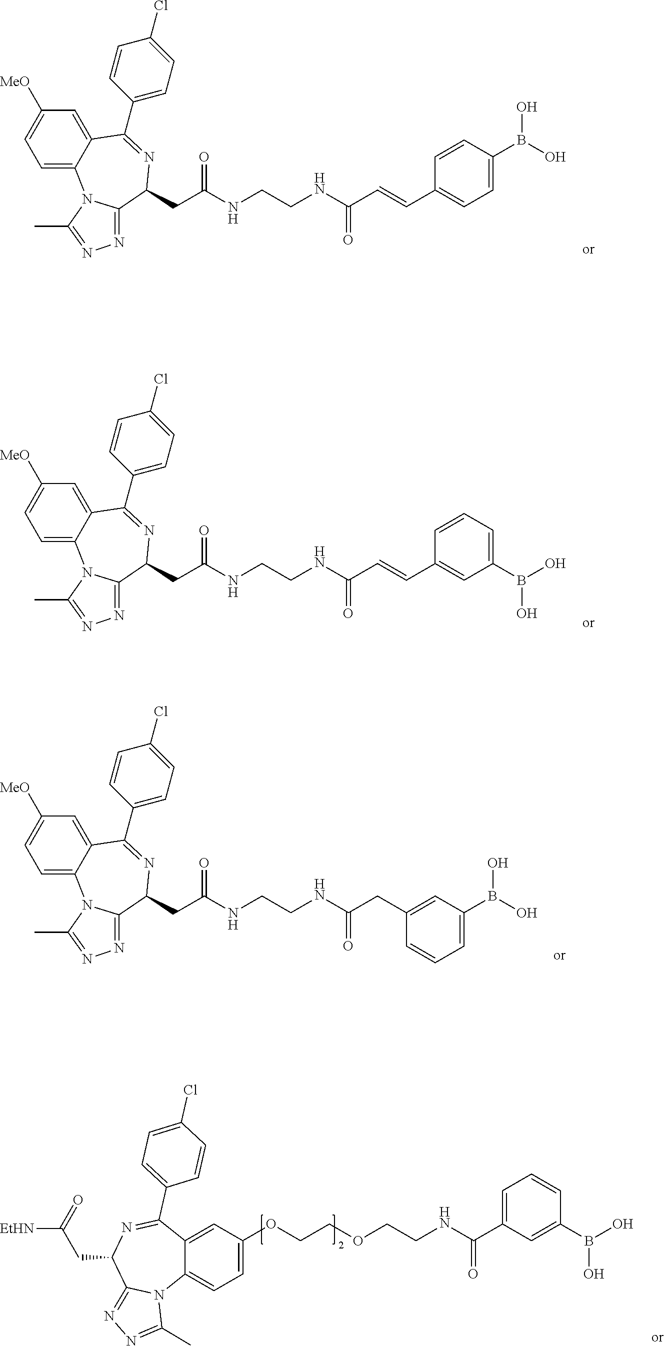

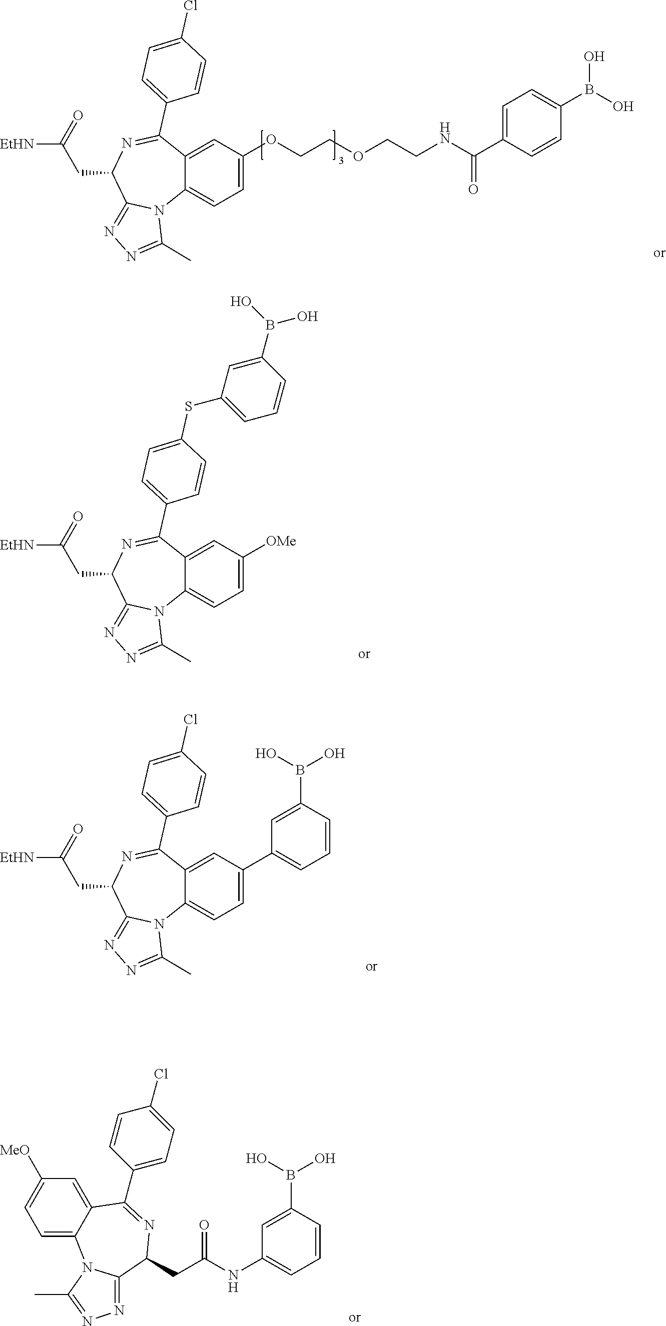

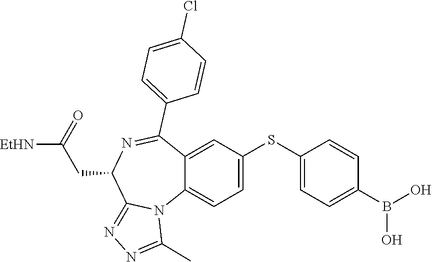

In another embodiment of the therapeutic composition of compounds, the E3ULB-C₁-L₁ moiety-containing first precursor compound has the following structure, or salts, enantiomers, stereoisomers, or polymorphs thereof:

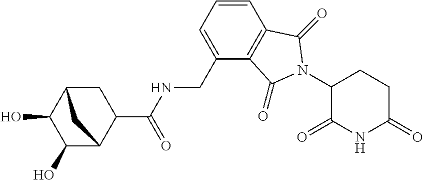 [SMILES: O=C1CCC(N2C(=O)c3cccc(CNC(=O)C4C[C@@H]5C[C@H]4[C@@H](O)[C@H]5O)c3C2=O)C(=O)N1]

and the TPB-C₂-L₂ moiety-containing second precursor compound is one of the following structures, or salts, enantiomers, stereoisomers, or polymorphs thereof:

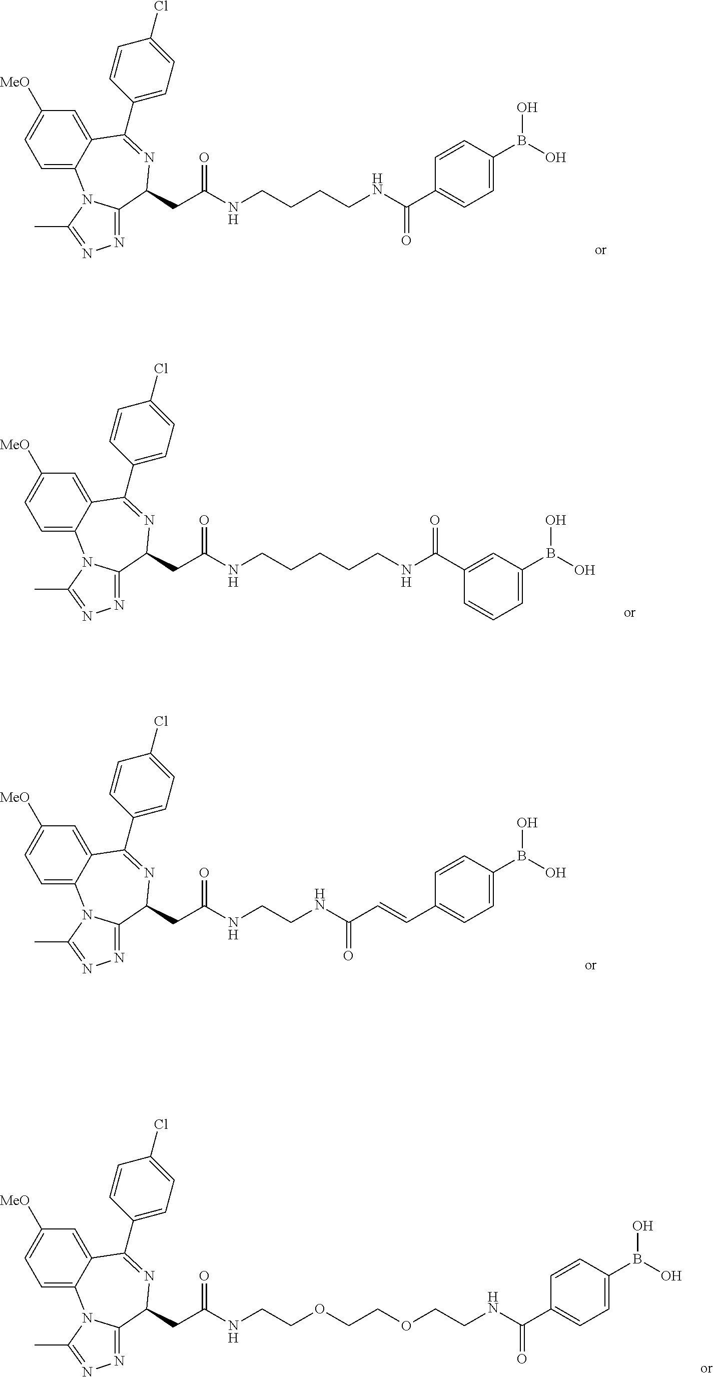 [SMILES: COc1ccc2c(c1)C(c1ccc(Cl)cc1)=N[C@@H](CC(=O)NCCCCNC(=O)c1ccc(B(O)O)cc1)c1nnc(C)n1-2 ; COc1ccc2c(c1)C(c1ccc(Cl)cc1)=N[C@@H](CC(=O)NCCCCCNC(=O)c1cccc(B(O)O)c1)c1nnc(C)n1-2 ; COc1ccc2c(c1)C(c1ccc(Cl)cc1)=N[C@@H](CC(=O)NCCNC(=O)/C=C/c1ccc(B(O)O)cc1)c1nnc(C)n1-2 ; COc1ccc2c(c1)C(c1ccc(Cl)cc1)=N[C@@H](CC(=O)NCCOCCOCCNC(=O)c1ccc(B(O)O)cc1)c1nnc(C)n1-2]

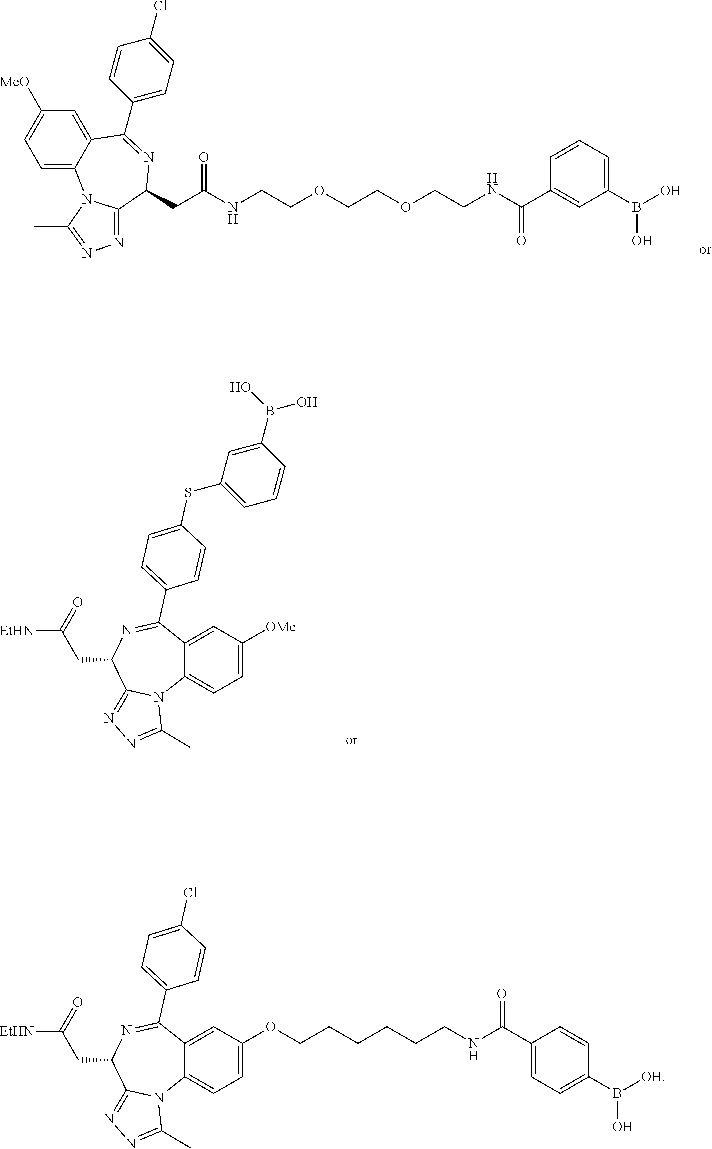

A second embodiment of an E3 ubiquitin ligase pharmacophore or ligand is one that binds to the VHL subunit of the CULLIN2 or CULLIN5 E3 ligase machinery. Such moieties have been successfully used within the PROTAC field, and often provide better selectivity in protein binding partner than those targeting CRBN (Fulcher et al., Open Biol. 7:170066 (2017); Chu et al., Cell Chem. Biol. 23(4):453-61 (2016); Cromm and Crews, Cell Chem. Biol. pii:S2451-9456(17)30187-3 (2017); Gadd et al., Nat Chem. Biol. 13(5):514-521 (2017), which are hereby incorporated by reference in their entirety).

A generic structure of a VHL ligand suitable for CURE-PRO degradation has the following structure, or salts, enantiomers, stereoisomers, or polymorphs thereof:

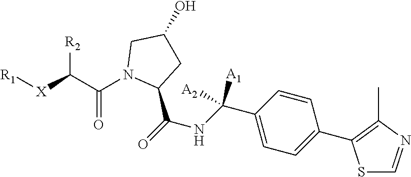

wherein
R₁ to R₂ are independently —H, —C₁₋₆ alkyl, or a bond to —C₁-L₁;
A₁ and A₂ are independently —H, —C₁₋₆ alkyl, C₁₋₆ alkoxy, alkyl amine, —C(O)NH₂, or a bond to —C₁-L₁; and
X is independently —H, C₁₋₆ alkyl, heteroalkyl, aryl, heteroaryl, alkyl(aryl), alkyl(heteroaryl), or a natural or unnatural amino acid;
   wherein one of R₁, R₂, A₁, or A₂ comprises a bond to —C₁-L₁. In one exemplary embodiment the compound has a formula of:

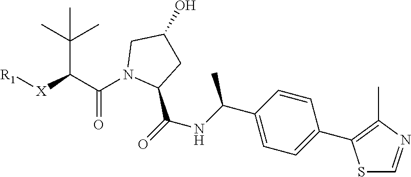

wherein, A₁ is a methyl group, A₂ is a proton, R₂ is a ⁱBu group, and R₁ comprises a bond to —C₁-L₁.

A second generic structure of a VHL ligand suitable for CURE-PRO degradation has the following structure, or salts, enantiomers, stereoisomers, or polymorphs thereof:

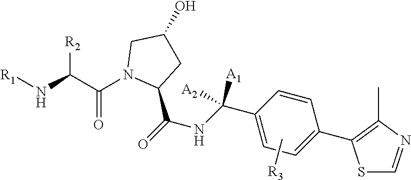

wherein
R₁ to R₃ are independently —H, —C₁₋₆ alkyl, aryl, heteroaryl, —C₁₋₆ alkyl aryl, —C₁₋₆ alkyl(heteroaryl), an amino acid or a bond to —C₁-L₁; and
A₁ and A₂ are independently —H, —C₁₋₆ alkyl, —C₁₋₆ alkoxy, alkyl amine, —CH₂C(O)OH; —CH₂C(O)NH₂, —C(O)NH₂, or a bond to —C₁-L₁;
   wherein one of R₁ to R₃, A₁, or A₂ comprises a bond to —C₁-L₁. In one exemplary embodiment, the compound has a formula of:

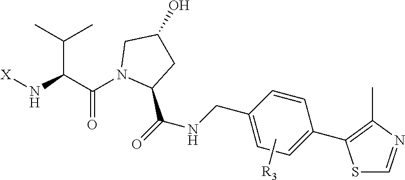

wherein A₁ and A₂ are each a hydrogen, and R₂ is an ⁱPr group, R₃ comprises —C₁-L₁, and X can be exemplified by:

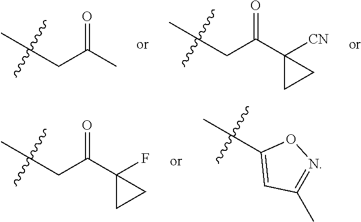

A third generic structure a VHL ligand suitable for CURE-PRO degradation has the following structure, or salts, enantiomers, stereoisomers, or polymorphs thereof:

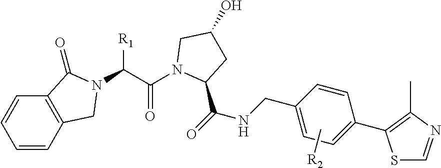

wherein
R₁ to R₂ are independently —H, —C₁₋₆ alkyl, or a bond to —C₁-L₁;
   wherein one of R₁ to R₂ comprises a bond to —C₁-L₁

Two further generic structures of VHL ligands suitable for CURE-PRO degradation have one of the following structures, or salts, enantiomers, stereoisomers, or polymorphs thereof:

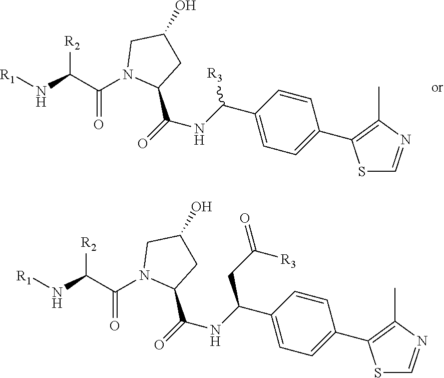

wherein
R₁ to R₃ are independently —H, —C₁₋₆ alkyl, heteroalkyl, aryl, heteroaryl, alkyl(aryl), alkyl(heteroaryl), natural or unnatural amino acid, or a bond to —C₁-L₁; wherein one of R₁ to R₃ comprises a bond to —C₁-L₁.

In an exemplary embodiment of the therapeutic composition of compounds, the E3ULB ubiquitin binding moiety that binds to the VHL subunit of the CULLIN2 or CULLIN5 E3 ligase machinery has the following structure, or salts, enantiomers, stereoisomers, or polymorphs thereof:

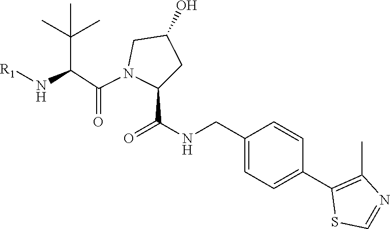

wherein
R₁ comprises a bond to —C₁-L₁.

In another embodiment of the therapeutic composition of compounds, the E3ULB-C₁-L₁ moiety-containing first precursor compound has the following structure, or salts, enantiomers, stereoisomers, or polymorphs thereof:

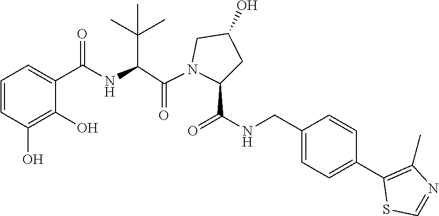 [SMILES: Cc1ncsc1-c1ccc(CNC(=O)[C@@H]2C[C@@H](O)CN2C(=O)[C@@H](NC(=O)c2cccc(O)c2O)C(C)(C)C)cc1]

and the TPB-C₂-L₂ moiety-containing second precursor compound has the following structure, or salts, enantiomers, stereoisomers, or polymorphs thereof:

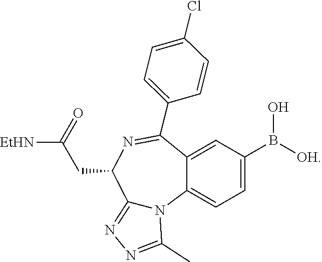

In a further exemplary embodiment the compound has a formula of:

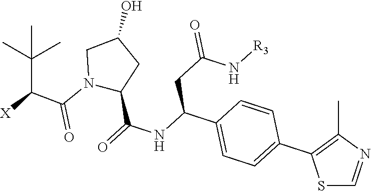

wherein R₃ comprises a bond to —C₁-L₁, and X can be exemplified by:

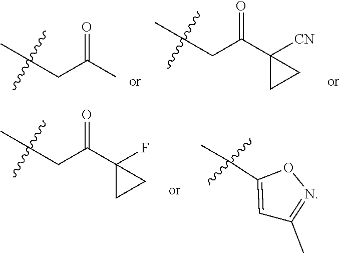

A third embodiment of an E3 ubiquitin ligase pharmacophore or ligand is one that binds to the MDM2 E3 ligase. Ligands targeting MDM2 have been successfully used within the PROTAC field, both for using MDM2 to target degradation of BRD4, as well as using CRBN to target the degradation of MDM2 (Hines et al., Cancer Res. 79(1):251-262 (2019); L₁ et al., J. Med. Chem. 62(2):448-466 (2019), which are hereby incorporated by reference in their entirety).

A generic structure of a MDM2 ligand suitable for CURE-PRO degradation has the following structure, or salts, enantiomers, stereoisomers, or polymorphs thereof:

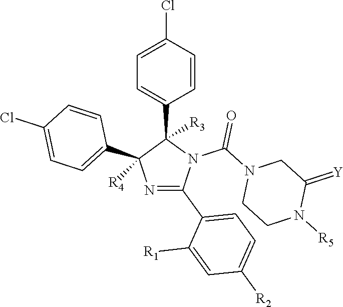

wherein
R₁ to R₅ are independently —H, —OH, —C₁₋₆ alkyl, —C₁₋₆ alkoxy, alkyl amine, aryl, heteroaryl, —C(O)NH₂, or a bond to —C₁-L₁; and
Y is independently H₂ or O;
   wherein one of R₁ to R₅ comprises a bond to —C₁-L₁.

In an exemplary embodiment, the generic MDM2 ligand may be depicted by:

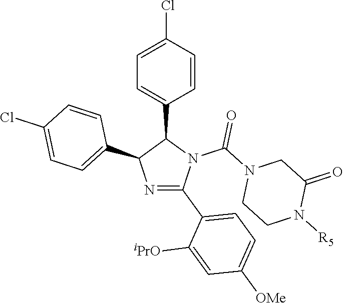

wherein R₅ comprises a bond to —C₁-L₁.

In a further embodiment of the therapeutic composition of compounds, the E3ULB ubiquitin binding moiety that binds to the MDM2 E3 ligase is one of the following structures, or salts, enantiomers, stereoisomers, or polymorphs thereof:

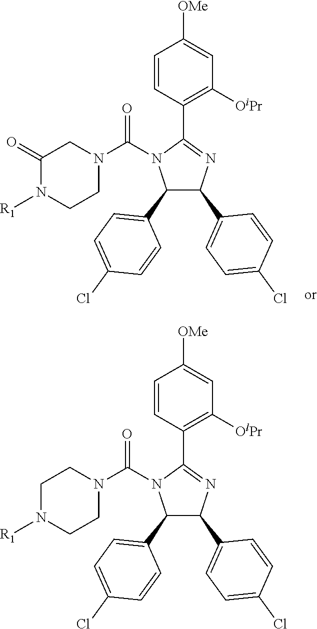

wherein
R₁ comprises a bond to —C₁-L₁.

Ligands targeting MDM2 have been successfully used within the PROTAC field (Skalniak et al., Expert Opin. Ther. Pat. 29(3):151-170 (2019), which is hereby incorporated by reference in its entirety). An exemplary ligand suitable for CURE-PRO has the following structure, or salts, enantiomers, stereoisomers, or polymorphs thereof:

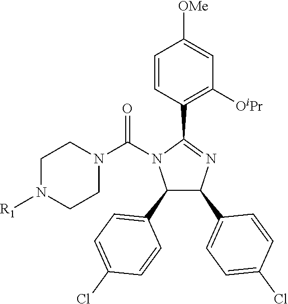

wherein
R₁ is a bond to —C₁-L₁.

In a further exemplary embodiment of the therapeutic composition of the CURE-PRO compounds, the E3ULB-C₁-L₁ moiety-containing first precursor compound has the following structure, or salts, enantiomers, stereoisomers, or polymorphs thereof:

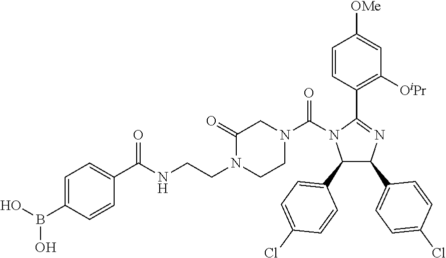 [SMILES: COc1ccc(C2=N[C@@H](c3ccc(Cl)cc3)[C@@H](c3ccc(Cl)cc3)N2C(=O)N2CCN(CCNC(=O)c3ccc(B(O)O)cc3)C(=O)C2)c(OC(C)C)c1]

and the TPB-C₂-L₂ moiety-containing second precursor compound has the following structure, or salts, enantiomers, stereoisomers, or polymorphs thereof:

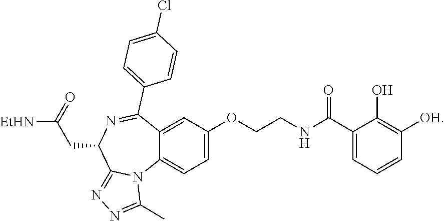

A further embodiment of an E3 ubiquitin ligase pharmacophore or ligand that binds to the MDM2 E3 ligase has the following structure, or salts, enantiomers, stereoisomers, or polymorphs thereof:

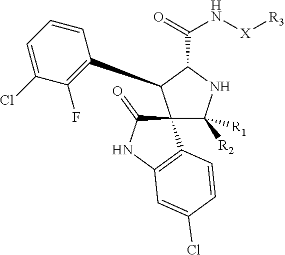

wherein
R₁ to R₃ are independently —H, —OH, —C₁₋₆ alkyl, —C₁₋₆ alkoxy, alkyl amine, aryl, heteroaryl, or a bond to —C₁-L₁; and
X is independently H₂, R₃, a carbocycle, heterocycle, aryl, heteroaryl, -alkyl(aryl), or -alkyl(heteroaryl) group; and
   wherein one of R₁ to R₃ comprises a bond to —C₁-L₁. In certain exemplary embodiments, X is:

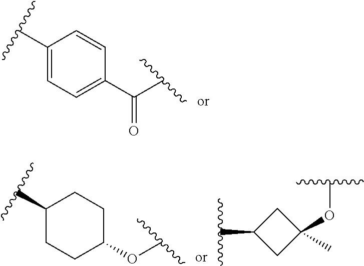

In one exemplary embodiment, the generic MDM2 ligand has the following structure, or salts, enantiomers, stereoisomers, or polymorphs thereof:

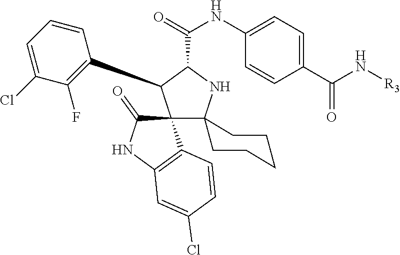

wherein R₃ comprises —C₁-L₁.

Another embodiment of an E3 ubiquitin ligase pharmacophore that binds to the MDM2 E3 ligase has the following structure, or salts, enantiomers, stereoisomers, or polymorphs thereof:

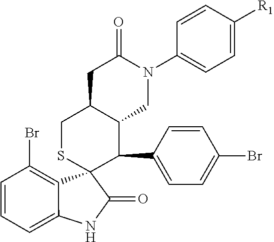

wherein R₁ comprises a bond to —C₁-L₁.

Ligands using MDM2 or targeting M1DM2 have been successfully used within the PROTAC field (Holzer et al., J Med. Chem. 58(16):6348-58 (2015), which is hereby incorporated by reference in its entirety). An exemplary ligand suitable for CURE-PRO has the following structure, or salts, enantiomers, stereoisomers, or polymorphs thereof:

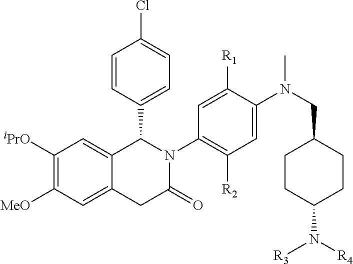

wherein
R₁ to R₄ are independently —H, —OH, —C₁₋₆ alkyl, —C₁₋₆ alkoxy, alkyl amine, aryl, heteroaryl, or a bond to —C₁-L₁; wherein one of R₁ to R₄ comprises a bond to —C₁-L₁.

In another embodiment, the generic MDM2 ligand may be depicted by:

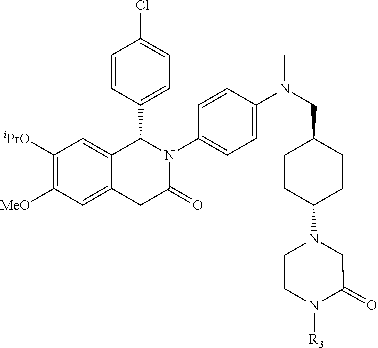

wherein R₃ comprises a bond to —C₁-L₁.

Additional ligands targeting MDM2 or inhibiting MDM2 include Spirooxindoles (Wang et al., J. Am. Chem. Soc., 135(19): 7223-7234 (2013), which is hereby incorporated by reference in its entirety). An exemplary ligand suitable for CURE-PRO has the following structure, or salts, enantiomers, stereoisomers, or polymorphs thereof:

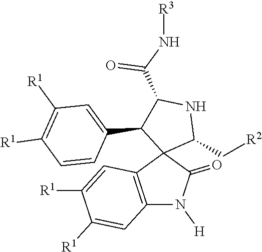

wherein
R¹ are independently —H, —OH, or halogen; and
R² and R³ are independently —H, —C₁₋₆ alkyl, —C₁₋₆ alkoxy, aryl, heteroaryl, alkyl amine, —C(O)NH₂, or a bond to —C₁-L₁, wherein one of R² or R₃ comprises a bond to —C₁-L₁.

Additional ligands include piperidinone inhibitors of the MDM2-p53 interaction (Sun et al., J. Med Chem., 57(4): 1454-1472 (2014), which is hereby incorporated by reference in its entirety). An exemplary ligand suitable for CURE-PRO has the following structure, or salts, enantiomers, stereoisomers, or polymorphs thereof:

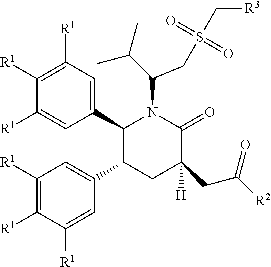

wherein
R¹ are independently —H, —OH, or halogen; and
R² and R³ are independently —H, —C₁₋₆ alkyl, —C₁₋₆ alkoxy, aryl, heteroaryl, alkyl amine, —C(O)NH₂, or a bond to —C₁-L₁, wherein one of R² or R₃ comprises a bond to —C₁-L₁.

Additional ligands include RG7388-based inhibitors of the MDM2-p53 interaction (Graves et al., J. Med Chem., 56(14) 5979-5983 (2013), which is hereby incorporated by reference in its entirety). An exemplary ligand suitable for CURE-PRO has the following structure, or salts, enantiomers, stereoisomers, or polymorphs thereof:

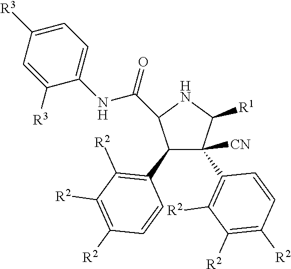

wherein
R₂ are independently —H, —OH, or halogen; and
R₁ and R³ are independently —H, —C₁₋₆ alkyl, —C₁₋₆ alkoxy, aryl, heteroaryl, alkyl amine, —C(O)NH₂, COOH, or a bond to —C₁-L₁, wherein one of R₁ or R³ comprises a bond to —C₁-L₁.

Additional ligands include tetra-substituted imidazole inhibitors of the MDM2-p53 interaction (Furet et al., Bioorg. Med Chem. Lett., 24 (9): 2110-2114 (2014), and Furet et al., Bioorg. Med Chem. Lett., 26(19): 4837-4841 (2016), which are hereby incorporated by reference in its entirety). An exemplary ligand suitable for CURE-PRO has the following structure, or salts, enantiomers, stereoisomers, or polymorphs thereof:

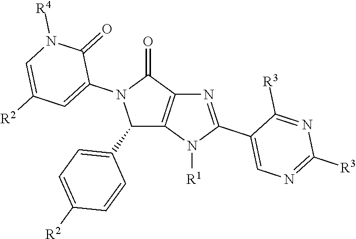

wherein
R² are independently —H, —OH, or halogen; and
R₁, R³ and R⁴ are independently —H, —C₁₋₆ alkyl, —C₁₋₆ alkoxy, aryl, heteroaryl, alkyl amine, —C(O)NH₂, or a bond to —C₁-L₁, wherein one of R₁, R³ or R⁴ comprises a bond to —C₁-L₁.

Additional ligands include Spirooxindoles inhibitors of the MDM2-p53 interaction (Bakarat et al., Biorg. Chem., 86: 598-604 (2019), which is hereby incorporated by reference in its entirety). An exemplary ligand suitable for CURE-PRO has the following structure, or salts, enantiomers, stereoisomers, or polymorphs thereof:

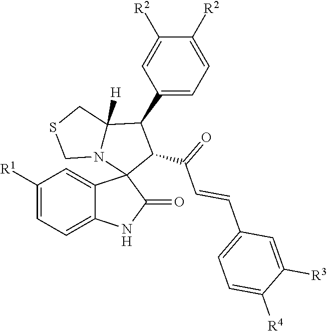

wherein
R₁ is —H, —OH, or halogen; and
R², R³ and R⁴ are independently —H, —C₁₋₆ alkyl, —C₁₋₆ alkoxy, aryl, heteroaryl, halogen, alkyl amine, —C(O)NH₂, or a bond to —C₁-L₁, wherein one of R², R³ or R₄ comprises a bond to —C₁-L₁.

Additional ligands include diastereomeric 2-thioxo-5H-dispiro[imidazolidine-4,3-pyrrolidine-2,3-indole]-2,5(1H)-dione inhibitors of the MDM2-p53 interaction (Ivanenkov et al., Bioorg. Med. Chem. Lett., 25(2): 404-409 (2015), which is hereby incorporated by reference in its entirety). An exemplary ligand suitable for CURE-PRO has the following structure, or salts, enantiomers, stereoisomers, or polymorphs thereof:

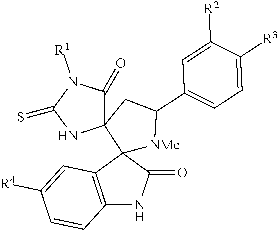

wherein
R¹ is —H, —C₁₋₆ alkyl, —C₁₋₆, aryl, heteroaryl, alkyl amine, —C(O)NH₂, or a bond to —C₁-L₁;
R² and R³ are independently —H, —C₁₋₆ alkyl, —C₁₋₆ alkoxy, aryl, heteroaryl, halogen, alkyl amine, —C(O)NH₂, or a bond to —C₁-L₁; and
R⁴ is —H, —OH, or halogen, wherein one of R¹, R² or R³ comprises a bond to —C₁-L₁.

Additional ligands include 1,4-Benzodiazepine-2,5-dione inhibitors of the MDM2-p53 interaction (Parks et al., Bioorg. Med. Chem. Lett., 15(3): 765-770 (2005), which is hereby incorporated by reference in its entirety). An exemplary ligand suitable for CURE-PRO has the following structure, or salts, enantiomers, stereoisomers, or polymorphs thereof:

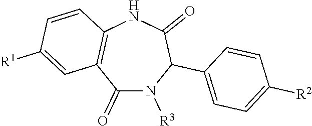

wherein
R¹ and R² are independently —H, —CH₃, —CH₂CH₃, —CH(CH₃)₂, —CF₃, —OCF₃, —OH, —OMe, or halogen; and
R₃ is a bond to —C₁-L₁.

Additional ligands include chromenotriazolopyrimidine inhibitors of the MDM2-p53 interaction (Beck et al., Bioorg. Med. Chem. Lett., 21(9): 2752-2755 (2011), which is hereby incorporated by reference in its entirety). An exemplary ligand suitable for CURE-PRO has the following structure, or salts, enantiomers, stereoisomers, or polymorphs thereof:

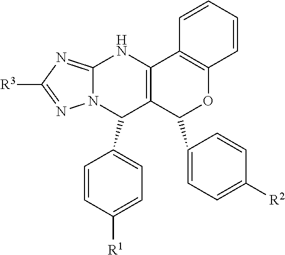

wherein
R¹ and R₂ are independently —H, —OH, or halogen; and
R³ is a bond to —C₁-L₁.

A fourth embodiment of an E3 ubiquitin ligase pharmacophore or ligand is one that binds to the DCAF subunit of the CULLIN4A or CULLIN4B E3 ligase machinery. Ligands targeting DCAF have been successfully used within the PROTAC field (Zoppi et al., J. Med Chem. 62(2):699-726 (2019), which is hereby incorporated by reference in its entirety). A generic structure for a DCAF ligand suitable for CURE-PRO degradation has the following structure, or salts, enantiomers, stereoisomers, or polymorphs thereof:

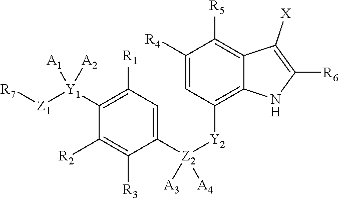

wherein
X is —H, -halogen, —CN, —CF₃, —OCF₃, —C₁₋₆ alkyl, or —C₁₋₆ alkoxy;
Y₁, Y₂, and Z₁, Z₂ are independently O, N, C, S, Si, P, or B;
A₁ to A₄ are independently —H, ═O, ═S, or —C₁₋₆ alkyl; and
R₁ to R₇ are independently —H, —OH, —C₁₋₆ alkyl, —C₁₋₆ alkoxy, alkyl amine, aryl, heteroaryl, or a bond to —C₁-L₁; and
   wherein one of R₁ to R₇ comprises a bond to —C₁-L₁.

In certain exemplary embodiments, the generic DCAF ligand has the following structure, or salts, enantiomers, stereoisomers, or polymorphs thereof:

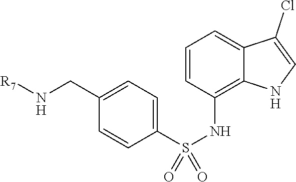

wherein R₇ comprises a bond to —C₁-L₁.

A second generic structure for a DCAF ligand suitable for CURE-PRO degradation has the following structure, or salts, enantiomers, stereoisomers, or polymorphs thereof:

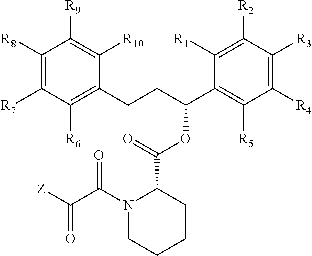

wherein
Z is —H, —OH, —C₁₋₆ alkyl, —C₁₋₆ alkoxy, alkyl amine, aryl, or heteroaryl; and
R₁ to R₁₀ are independently —H, —OH, —C₁₋₆ alkyl, —C₁₋₆ alkoxy, alkyl amine, aryl, heteroaryl, or a bond to —C₁-L₁;
   wherein one of R₁ to R₁₀ comprises a bond to —C₁-L₁.

In an exemplary embodiment, this second generic DCAF ligand has the following structure, or salts, enantiomers, stereoisomers, or polymorphs thereof:

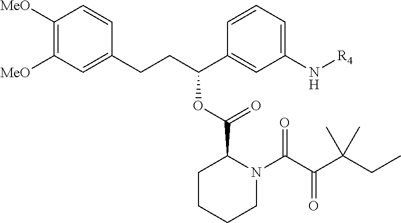

wherein R₄ comprises a bond to —C₁-L₁.

A fifth embodiment of an E3 ubiquitin ligase pharmacophore or ligand is one that binds to an inhibitor of apoptosis proteins E3 ubiquitin ligase, such as cIAP, XIAP, or others in the family. Ligands targeting the IAP proteins have been successfully used within the PROTAC field (Ohoka et al., J. Biol. Chem. 292(11):4556-4570 (2017); Okuhira et al., Mol. Pharmacol. 91(3):159-166 (2017); and Ottis and Crews, ACS Chem. Biol. 12(4):892-898 (2017), which are hereby incorporated by reference in their entirety). In certain exemplary embodiments, the generic IAP protein ligand is a derivative of bestatin, and has the following structure, or salts, enantiomers, stereoisomers, or polymorphs thereof:

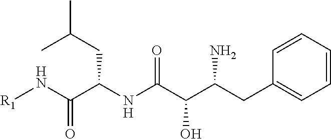

wherein R₁ comprises a bond to —C₁-L₁.

In a second exemplary embodiment, the generic IAP protein ligand is derived from the compound MV1, and has the following structure, or salts, enantiomers, stereoisomers, or polymorphs thereof:

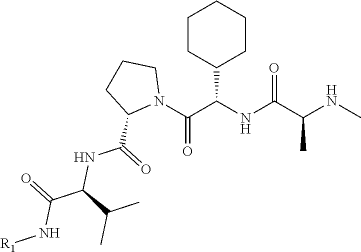

wherein R₁ comprises a bond to —C₁-L₁.

In a third exemplary embodiment, the generic IAP protein ligand is derived from the compound LAL161, and has the following structure, or salts, enantiomers, stereoisomers, or polymorphs thereof:

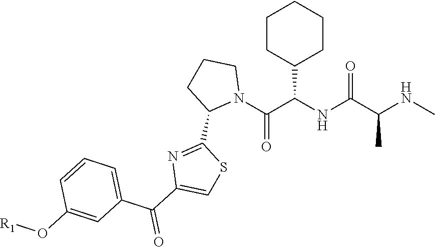

wherein R₁ comprises a bond to —C₁-L₁.

A sixth embodiment of an E3 ubiquitin ligase pharmacophore or ligand is one that binds to the KEAP1 subunit of the CULLIN3 E3 ligase machinery. Ligands targeting KEAP1 have been successfully used within the PROTAC field (Mészáros et al., Sci. Signal. 10(470) (2017); Bulatov and Ciulli Biochem. J. 467(3):365-86 (2015); Sun et al., Exp. Opin. Ther. Pat 27:763-785 (2017), which are hereby incorporated by reference in their entirety). A generic structure for a KEAP1 ligand suitable for CURE-PRO degradation has the following structure, or salts, enantiomers, stereoisomers, or polymorphs thereof:

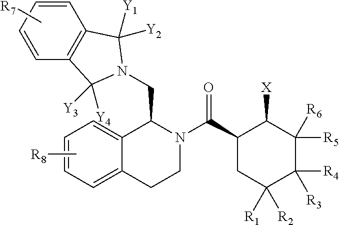

wherein
R₁ to R₇ are independently —H, —C₁₋₆ alkyl, —C₁₋₆ alkoxy, alkyl amine, aryl, heteroaryl, or a bond to —C₁-L₁;
R₈ is —H; —C₁₋₆ alkyl, —C₁₋₆ alkoxy, a carbocycle, heterocycle, aryl, heteroaryl, -alkyl(aryl), or -alkyl(heteroaryl) group, a carboxylic acid, or bond to —C₁-L₁;
X is a carboxylic acid, ether moiety, ester moiety, amide moiety, aromatic moiety, heteroaromatic moiety; and
Y₁ to Y₄ are independently —H, ═O, ═S, —C₁₋₆ alkyl; wherein one of R₁ to R₈ comprises a bond to —C₁-L₁;

In a certain exemplary embodiment, the generic KEAP1 ligand has the following structure, or salts, enantiomers, stereoisomers, or polymorphs thereof:

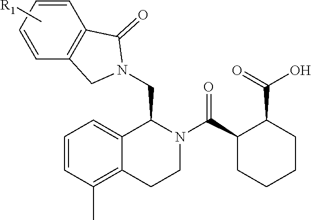

wherein R₁ comprises a bond to a bond to —C₁-L₁.

In a certain exemplary embodiment, the generic KEAP1 ligand may be depicted by:

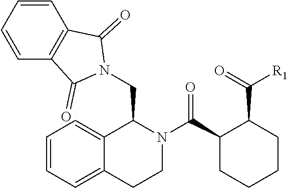

wherein R₁ comprises a bond to —C₁-L₁.

A second generic structure for a KEAP1 ligand suitable for CURE-PRO degradation has the following structure, or salts, enantiomers, stereoisomers, or polymorphs thereof:

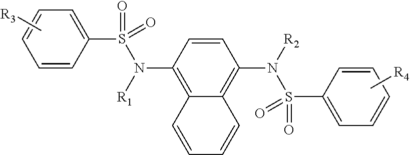

wherein
R₁ and R₂ are independently —H, —C₁₋₆ alkyl, —C₁₋₆ alkoxy, alkyl amine, a carbocycle, heterocycle, aryl, heteroaryl, -alkyl(aryl), or -alkyl(heteroaryl) group, a carboxylic acid, —CH₂C(O)X, or a bond to —C₁-L₁;
X is —OH, —C₁₋₆ alkyl, —C₁₋₆ alkoxy, —NH₂, —NHCOCH₃, a heterocycle, aryl, heteroaryl, -alkyl(aryl), or -alkyl(heteroaryl) group; and
R₃ and R₄ are independently —H; —C₁₋₆ alkyl, —C₁₋₆ alkoxy, or a bond to —C₁-L₁; and wherein one of R₁ to R₄ comprises a bond to —C₁-L₁.

In a further embodiment, the generic KEAP1 ligand has the following structure, or salts, enantiomers, stereoisomers, or polymorphs thereof:

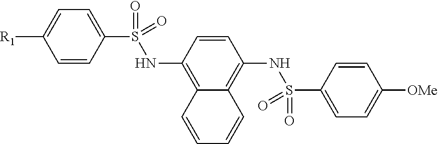

wherein R₁ comprises a bond to —C₁-L₁.

A third generic structure for a KEAP1 ligand suitable for CURE-PRO degradation is depicted by:

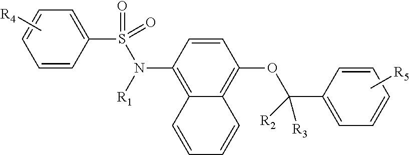

wherein
R₁-R₃ are independently be —H, —C₁₋₆ alkyl, —C₁₋₆ alkoxy, alkyl amine, a carbocycle, heterocycle, aryl, heteroaryl, -alkyl(aryl), -alkyl(heteroaryl) group, a carboxylic acid, or —CH₂C(O)X;
X is —OH, —C₁₋₆ alkyl, —C₁₋₆ alkoxy, —NH₂, —NHCOCH₃, a heterocycle, aryl, heteroaryl, -alkyl(aryl), or -alkyl(heteroaryl) group;
R₄-R₅ are independently —H, —C₁₋₆ alkyl, —C₁₋₆ alkoxy, alkyl amine, a carbocycle, heterocycle, aryl, heteroaryl, -alkyl(aryl), -alkyl(heteroaryl) group, a carboxylic acid, —OY, —NHY, —C(O)Y, OC(O)Y, NHC(O)Y, or a bond to —C₁-L₁; and
Y is —H, —C₁₋₆ alkyl, —C₁₋₆ alkoxy, alkyl amine, aryl, or heteroaryl; and
   wherein one of R₄ to R₅ comprises a bond to —C₁-L₁.

A fourth generic structure for a KEAP1 ligand suitable for CURE-PRO degradation is depicted by:

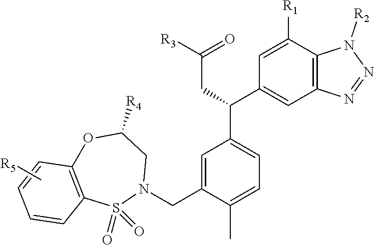

wherein
R₁ to R₄ are independently —H; —C₁₋₆ alkyl, —C₁₋₆ alkoxy, alkyl amine, aryl, heteroaryl, —OX, —NHX, —C(O)X, —OC(O)X, —NHC(O)X, or a bond to —C₁-L₁;
R₅ is —H; —C₁₋₆ alkyl, —C₁₋₆ alkoxy, alkyl amine, a carbocycle, heterocycle, aryl, heteroaryl, -alkyl(aryl), or -alkyl(heteroaryl) group, a carboxylic acid, —OX, —NHX, —C(O)X, —OC(O)X, —NHC(O)X, or a bond to —C₁-L₁; and
X is —H, —C₁₋₆ alkyl, —C₁₋₆ alkoxy, alkyl amine, aryl, or heteroaryl; and
   wherein one of R₁ to R₅ comprises a bond to —C₁-L₁.

A sixth embodiment of an E3 ubiquitin ligase pharmacophore or ligand is one that binds to the β-TrCP1 subunit of the CULLIN1 E3 ligase machinery. Ligands targeting β-TrCP1 have been successfully used within the PROTAC (Sakamoto et al., Mol. Cell Proteomics 2(12):1350-8, (2003), which is hereby incorporated by reference in its entirety). A generic structure for a R-TrCP1 ligand suitable for CURE-PRO degradation is one of the following structures, or salts, enantiomers, stereoisomers, or polymorphs thereof:

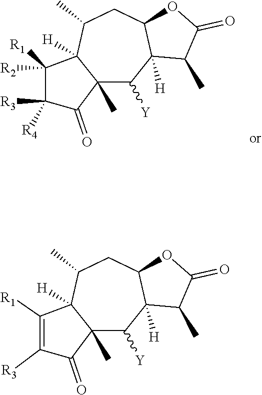

wherein
R₁ to R₄ are independently —H, —C₁₋₆ alkyl, —C₁₋₆ alkoxy, alkyl amine, aryl, heteroaryl, —OX, —NHX, —C(O)X, —OC(O)X, —NHC(O)X, or a bond to —C₁-L₁;
Y is —H, —C₁₋₆ alkyl, —C₁₋₆ alkoxy, alkyl amine, aryl, heteroaryl, —OX, —NHX, —C(O)X, —OC(O)X, or —NHC(O)X; and
X is independently —H, —C₁₋₆ alkyl, —C₁₋₆ alkoxy, alkyl amine, aryl, or heteroaryl; wherein one of R₁ to R₄ comprises a bond to —C₁-L₁.

In one embodiment, the generic β-TrCP1 ligand has the following structure, or salts, enantiomers, stereoisomers, or polymorphs thereof:

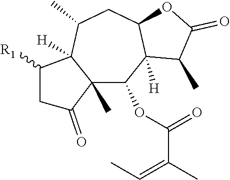

wherein R₁ comprises a bond to —C₁-L₁.

A seventh embodiment of an E3 ubiquitin ligase pharmacophore or ligand is one that binds to the SPOP subunit of the CULLIN3 E3 ligase machinery. A generic structure for a SPOP ligand suitable for CURE-PRO degradation has the following structure, or salts, enantiomers, stereoisomers, or polymorphs thereof:

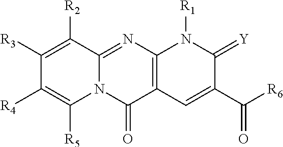

wherein
R₁ to R₅ are independently —H, —C₁₋₆ alkyl, —C₁₋₆ alkoxy, alkyl amine, aryl, heteroaryl, —OX, —NHX, —C(O)X, —OC(O)X, —NHC(O)X, or a bond to —C₁-L₁;
R₆ is independently —C₁₋₆ alkyl, —C₁₋₆ alkoxy, alkyl amine, aryl, heteroaryl, —OX, —NHX, —C(O)X, or a bond to —C₁-L₁;
X is —H, —C₁₋₆ alkyl, —C₁₋₆ alkoxy, alkyl amine, a heterocycle, aryl, heteroaryl, alkyl(aryl), or -alkyl(heteroaryl) group; and
Y is H₂, O, N, or S;
   wherein one of R₁ to R₆ comprises a bond to —C₁-L₁.

In a certain exemplary embodiment, the generic SPOP ligand has the following structure, or salts, enantiomers, stereoisomers, or polymorphs thereof:

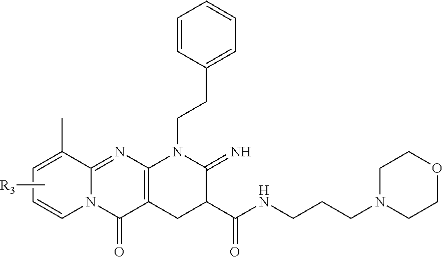

wherein R₃ comprises a bond to —C₁-L₁.

An eighth embodiment of an E3 ubiquitin ligase pharmacophore or ligand is one that binds to the CBL E3 ligase machinery. A generic structure for a CBL ligand suitable for CURE-PRO degradation has the following structure, or salts, enantiomers, stereoisomers, or polymorphs thereof:

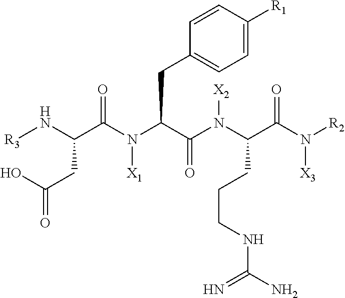

wherein
R₁ is —H, —OH, —CO₂H, —CO₂⁻, sulfate, nitrate, phosphate, —SO₂NH₂, or —C(O)NH₂;
R₂ to R₃ can independently be —H, —C₁₋₆ alkyl, —C₁₋₆ alkoxy, alkyl amine, aryl, heteroaryl, —OX, —NHX, —C(O)X, —OC(O)X, —NHC(O)X, or a bond to —C₁-L₁;
X is —H, —C₁₋₆ alkyl, —C₁₋₆ alkoxy, alkyl amine, a heterocycle, aryl, heteroaryl, -alkyl(aryl), or -alkyl(heteroaryl) group; and
X₁ to X₃ are independently —H, —CH₃, —CF₃;
   wherein one of R₂ to R₃ comprises a bond to —C₁-L₁.

In an exemplary embodiment, the generic CBL ligand has the following structure, or salts, enantiomers, stereoisomers, or polymorphs thereof:

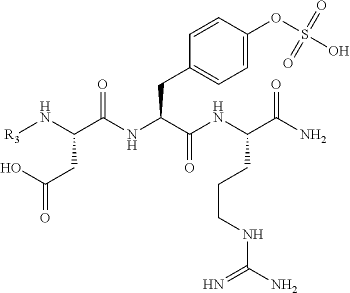

wherein R₃ comprises a bond to —C₁-L₁.

A ninth embodiment of an E3 ubiquitin ligase pharmacophore or ligand is one that binds to the ITCH E3 ligase machinery. A generic structure for an ITCH ligand suitable for CURE-PRO degradation has the following structure, or salts, enantiomers, stereoisomers, or polymorphs thereof:

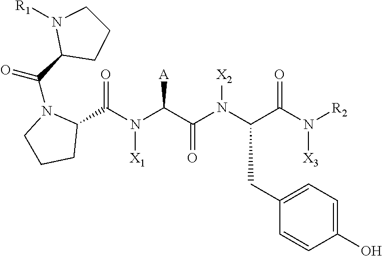

wherein
R₁ to R₂ are independently H, —C₁₋₆ alkyl, —C₁₋₆ alkoxy, alkyl amine, aryl, heteroaryl, —OX, —NHX, —C(O)X, —OC(O)X, —NHC(O)X, or a bond to —C₁-L₁;
X is —H, —C₁₋₆ alkyl, —C₁₋₆ alkoxy, alkyl amine, a heterocycle, aryl, heteroaryl, -alkyl(aryl), or -alkyl(heteroaryl) group;
A is the sidechain of any natural or unnatural amino acid, including glycine, or —H; and
X₁ to X₃ are independently —H, —CH₃, or —CF₃;
   wherein one of R₁ to R₂ comprises a bond to —C₁-L₁.

In a certain exemplary embodiment, the generic ITCH ligand has the following structure, or salts, enantiomers, stereoisomers, or polymorphs thereof:

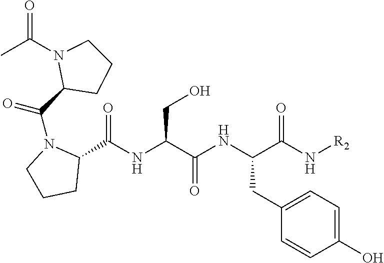

wherein R₂ comprises a bond to —C₁-L₁.

A tenth embodiment of an E3 ubiquitin ligase pharmacophore or ligand is one that binds to the Ring Finger Protein (RNF) E3 ligase machinery (Ward et al., ACS Chem. Biol. 14, 11, 2430-2440 (2019), which is hereby incorporated by reference in its entirety). A generic structure that binds to the RNF4 E3 ligase and is suitable for CURE-PRO degradation has the following structure, or salts, enantiomers, stereoisomers, or polymorphs thereof:

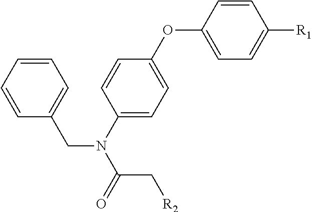

wherein
R₁ to R₂ are independently —H, -halogen, —C₁₋₆ alkyl, —C₁₋₆ alkoxy, —CF₃, or a bond to —C₁-L₁;
   wherein one of R₁ to R₂ comprises a bond to —C₁-L₁.

A second generic structure that binds to the RNF114 E3 ligase machinery (Spradlin et al., Nature Chemical Biology 15:747-755 (2019), which is hereby incorporated by reference in its entirety) and is suitable for CURE-PRO degradation has the following structure, or salts, enantiomers, stereoisomers, or polymorphs thereof:

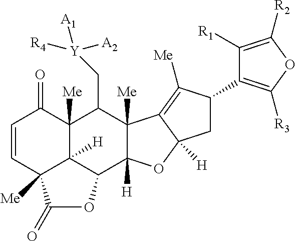

wherein
R₁ to R₄ are independently —H, —C₁₋₆ alkyl, —C₁₋₆ alkoxy, aryl, heteroaryl, neopentyl, acyl, -alkyl(aryl)-alkyl(heteroaryl), or a bond to —C₁-L₁;
Y is O, N, C, S, Si, P, or B; and
A₁ and A₂ are independently —H, ═O, ═S, -Me, or -Et;
   wherein one of R₁ to R₄ comprises a bond to —C₁-L₁.

In certain exemplary embodiments, the generic RNF 114 ligand has the following structure, or salts, enantiomers, stereoisomers, or polymorphs thereof:

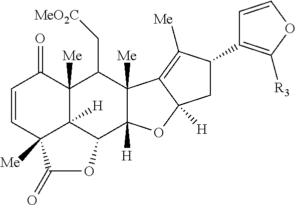

wherein R₃ comprises a bond to —C₁-L₁.

An eleventh embodiment of an E3 ubiquitin ligase pharmacophore or ligand is one that binds to either the CDH1 or CDC20 E3 ligase machinery. A generic structure for these ligands that is suitable for CURE-PRO degradation has the following structure, or salts, enantiomers, stereoisomers, or polymorphs thereof:

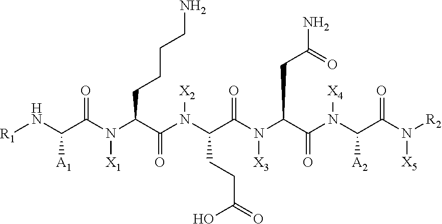

wherein
A₁ and A₂ are independently the sidechain of any natural or unnatural amino acid, including glycine, or —H;
X₁ to X₅ are independently —H, —CH₃, or —CF₃;
R₁ to R₂ are independently —H, —C₁₋₆ alkyl, —C₁₋₆ alkoxy, alkyl amine, aryl, heteroaryl, —OX, —NHX, —C(O)X, —OC(O)X, —NHC(O)X, or a bond to —C₁-L₁; and
X is —H, —C₁₋₆ alkyl, —C₁₋₆ alkoxy, alkyl amine, a heterocycle, aryl, heteroaryl, -alkyl(aryl), or -alkyl(heteroaryl) group;
   wherein one of R₁ to R₂ comprises a bond to —C₁-L₁.

In a certain exemplary embodiment, the generic CDH1 ligand has the following structure, or salts, enantiomers stereoisomers, or polymorphs thereof:

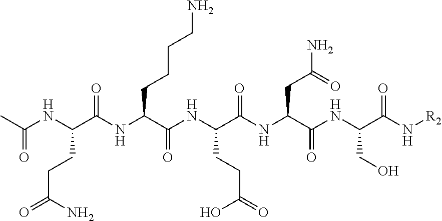

wherein R₂ comprises a bond to —C₁-L₁. In an additional exemplary embodiment, the generic CDC20 ligand has the following structure, or salts, enantiomers, stereoisomers, or polymorphs thereof:

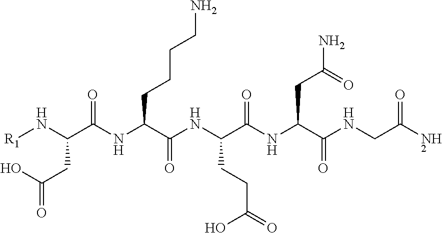

wherein R₁ comprises a bond to —C₁-L₁.

A twelfth embodiment of an E3 ubiquitin ligase pharmacophore or ligand is one that binds to the aryl hydrocarbon receptor (AhR) subunit of the CULLIN4B E3 ligase machinery (Ohoka N, et al., ACS Chem. Biol. 14(12):2822-2832 (2019), which is hereby incorporated by reference in its entirety). A generic structure for an AhR ligand suitable for CURE-PRO degradation has the following structure, or salts, enantiomers, stereoisomers, or polymorphs thereof:

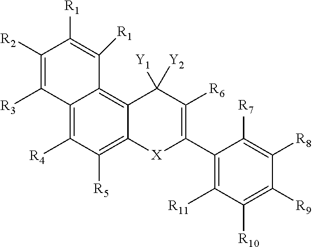

wherein
X is O, NH, CH₂, or S,
Y₁ and Y₂ are independently —H, ═O, ═S, -Me, and
R₁ to R₁₁ are independently —H, —C₁₋₆ alkyl, —C₁₋₆ alkoxy, aryl, heteroaryl, alkyl amine, or a bond to —C₁-L₁;
wherein one of R₁ to R₁₁, or one of Y₁ or Y₂ comprises a bond to —C₁-L₁;
or has the following structure, or salts, enantiomers, stereoisomers, or polymorphs thereof:

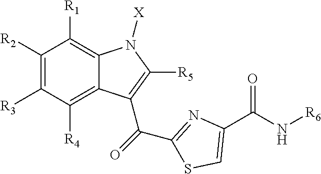

wherein
X is —H, —C₁₋₆ alkyl, aryl, —C₁₋₆ alkoxy, or alkyl amine, or a bond to —C₁-L; and
R₁ to R₆ are independently be —H, —C₁₋₆ alkyl, aryl, neopentyl, —C₁₋₆ alkoxy, or -alkyl amine, or a bond to —C₁-L₁;
wherein one of R₁ to R₆ or X comprises a bond to —C₁-L₁.

In an exemplary embodiment of the therapeutic composition of compounds, the E3ULB ubiquitin binding moiety that binds to the aryl hydrocarbon receptor (AhR) subunit of the CULLIN4B E3 ligase machinery has of the following structure, or salts, enantiomers, stereoisomers, or polymorphs thereof:

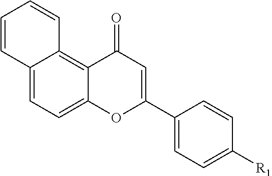

wherein R₁ comprises a bond to —C₁-L₁.

In another exemplary embodiment of the therapeutic composition of compounds, the E3ULB ubiquitin binding moiety that binds to the aryl hydrocarbon receptor (AhR) subunit of the CULLIN4B E3 ligase machinery is has the following structure, or salts, enantiomers, stereoisomers, or polymorphs thereof:

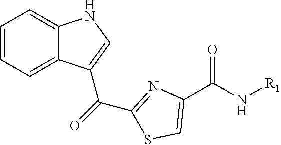

wherein R₁ comprises a bond to —C₁-L₁.

In a further embodiment, the E3 ligase ligand may comprise two or more connectors attached to one or more linker elements. The linker elements may covalently bond with partner linker elements connected to a single target ligand or two or more target ligands (Testa et al., Angew. Chem. Int. Ed. 59(4):1727-1734 (2020), which is hereby incorporated by reference in its entirety). For example, two target ligands that bind to a homodimeric protein target may comprise of linker elements that bind either a single linker element, or two independent linker elements on an E3 ligase ligand to recruit the E3 ligase machinery for subsequent ubiquitination of the target homodimer. Alternatively, two separate target ligands bind a heteromeric complex and recruit an E3 ligase ligand only when said proteins are in the heteromeric complex.

In one embodiment of the therapeutic composition of the present application, the TPB binding moiety has a dissociation constant less than 300 μM, less than 100 μM, less than 30 μM, less than 10 μM, less than 3 μM, less than 1 μM, less than 300 nM, or less than 100 nM when binding to a BET domain protein.

A second aspect of the present application relates to a method of binding to and redirecting the specificity of an E3 ubiquitin ligase, an E3 ubiquitin ligase complex, or subunit thereof to induce the ubiquitination and degradation of a BET domain protein in a biological sample. The method includes contacting the sample with the therapeutic composition of the present application.

A further aspect of the present application relates to a method of providing the therapeutic composition to maximize the therapeutic efficacy of the composition. In some cases, it may be desirable to use a lower concentration of the E3ULB-C₁-L₁ compound so as not to interfere with the housekeeping functions of the E3 ubiquitin ligase, an E3 ubiquitin ligase complex, or subunit thereof as they carry out their normal cellular function in the ubiquitination and degradation of mis-folded, or biologically tagged or cleaved proteins. Addition of 1.5-fold, 2-fold, 3-fold, 4-fold, 5-fold, 7.5-fold, 10-fold, 15-fold 20-fold, 30-fold, 50-fold, 75-fold, 100-fold or higher concentration or molar amount of the TPB-C₂-L₂ compound over the E3ULB-C₁-L₁ compound may be used to improve the ubiquitination and degradation of a BET domain protein in a biological sample, above and beyond a 1:1 ratio of the two compounds.

A further aspect of the present application relates to a method of providing the therapeutic composition to maximize the therapeutic efficacy of the composition, and also to overcome mutational escape that might arise when the patient is exposed to either the BET domain ligand alone, or a PROTAC comprising of a BET domain ligand (i.e. JQ1) and an E3 ubiquitin ligase complex ligand (i.e. CRBN-binding ligand). A recent study has shown acquired resistance to both VHL- and CRBN-based BET PROTACs in AML cell lines, following chronic exposure (Zhang et al., Mol. Cancer Ther. 18(7):1302-1311 (2019), which is hereby incorporated by reference in its entirety). Withdrawing BET PROTACs from chronically treated cells did not restore sensitivity, indicating a stable genomic alteration. Furthermore, BET PROTACs failed to alter MYC expression in resistant cells, despite marked BRD4 degradation (Pawar et al., Cell Rep. 22(9):2236-2245 (2018), which is hereby incorporated by reference in its entirety). Interestingly, the cells resistant to CRBN-based PROTACs remained sensitive to VHL-based PROTACs, and vice versa, with resistance arising from genetic mutations of the E3 ligase complex, while the downstream ubiquitin-proteasome system (UPS) remained functional (Zhang. et al., Mol Cancer Ther. 18(7):1302-1311 (2019), which is hereby incorporated by reference in its entirety). Indeed, mutations in the CUL2 gene, a component of the VHL-CRL complex, or the CRBN gene mediate resistance to VHL- or CRBN-based BET PROTACs, respectively, highlighting the potential contributions of E3 ligase complexes in acquired resistance to PROTACs in leukemia. The present application provides a unique opportunity to overcome this deficiency, since CURE-PROs do not exhibit the “hook effect”, which is a severe limitation of PROTACs (Bondenon et al., Cell Chem. Biol. 25(1):78-87 (2018), which is hereby incorporated by reference in its entirety). By combining 3 CURE-PRO compounds, one that binds the target BET domain protein (i.e. BRD4), and the other two that bind two different E3 ligase complexes (i.e. CRBN and VHL), a therapeutic composition may be used to overcome mutational escape. The BET domain ligand compound (TPB₁—C₃-L₃) may partner with either the CRBN ligand partner (E3ULB₁—C₁-L₁) or the VHL ligand partner (E3ULB₂—C₂-L₂) to induce proximity ubiquination and subsequent degradation of the target BET domain protein. Thus, in the general form, the therapeutic composition comprises: a first precursor compound having the chemical structure: E3ULB₁—C₁-L₁, and a second precursor compound having the chemical structure: E3ULB₂—C₂-L₂, and a third precursor compound having the chemical structure TPB₁—C₃-L₃, and where either L₁ or L₂ can form reversible covalent bonds (RCBs) with L₃. Further, addition of 1.5-fold, 2-fold, 3-fold, 4-fold, 5-fold, 7.5-fold, 10-fold, 15-fold 20-fold, 30-fold, 50-fold, 75-fold, 100-fold or higher concentration or molar amount of the TPB₁—C₃-L₃ compound over either the E3ULB₁—C₁-L₁ or the E3ULB₂-C₂-L₂ compound may be used to improve the ubiquitination and degradation of a BET domain protein in a biological sample, above and beyond a 1:1:1 ratio of the three compounds.

A further aspect of the present application relates to a method of providing the therapeutic composition to maximize the therapeutic efficacy of the composition, and also to overcome mutational escape that might arise when the patient is exposed to either the BET domain ligand alone, or a PROTAC comprising of a BET domain ligand (i.e. JQ1) and an E3 ubiquitin ligase complex ligand (i.e. CRBN-binding ligand). Traditional drugs are based on occupancy, and generally need to occupy 80% to 90% or more of the protein target to achieve a therapeutic effect. However, a mutation in the binding pocket that reduces occupancy to just 50% may be sufficient to enable the cancer to mutationally escape the drug action. Often, with occupancy-driven therapeutics, there is a need to drive drug binding to the nanomolar or even picomolar level, such that the compound still binds a protein even if it has a mutation in the binding pocket. However, such tight binding molecules come with the risk of off-target binding. In contrast, PROTACs and CURE-PRO's need to bind the protein target just long enough to bring it into proximity to the E3 ligase machinery to effect ubiquitination to mark the target for degradation, and thus are more tolerant of mutations that would escape from traditional occupancy drugs. Indeed, PROTACs targeted against an oncogenic kinase (BTK) or a viral protein (HepC NS3/4a protease) suggest that they can overcome mutational escape (Buhimschi et al., Biochemistry. 57(26):3564-3575 (2018); de Wispelaere et al., Nat. Commun. 10(1):3468 (2019), which are hereby incorporated by reference in their entirety). CURE-PRO's present additional opportunities to overcome mutational escape by using two different ligands. In one embodiment, two different ligands may be used that bind the same pocket slightly differently, such that a given mutation may lessen binding of one but not the other ligand. In another embodiment, two different ligands may be used, one that binds the wild-type pocket, while the other that binds the mutant pocket, such that both wild-type and mutant proteins are covered. In another embodiment, two different ligands may be used, one that binds one pocket or groove in the protein, while the other binds a second pocket or groove in the protein, such that a mutation in one pocket or groove may lessen binding of one but not the other ligand. For example, there are a number of potential ligands that bind the BET domain of BRD4, and two of these (i.e. TPB₁—C₂-L₂, and TPB₂—C₃-L₃) may be used simultaneously with a CRBN binding ligand (i.e. E3ULB₁—C₁-L₁). Thus, in the general form, the therapeutic composition comprises: a first compound having the chemical structure: E3ULB₁—C₁-L₁, and a second compound having the chemical structure: TPB₁—C₂-L₂, and a third compound having the chemical structure: TPB₂-C₃-L₃, and where L₁ can form RCBs with either L₂ or L₃. Further, addition of 1.5-fold, 2-fold, 3-fold, 4-fold, 5-fold, 7.5-fold, 10-fold, 15-fold 20-fold, 30-fold, 50-fold, 75-fold, 100-fold or higher concentration or molar amount of either the TPB₁—C₂-L₂, or the TPB₂—C₃-L₃ compound, or both, over the E3ULB₁—C₁-L₁ compound may be used to improve the ubiquitination and degradation of a BET domain wild-type and or mutant protein in a biological sample, above and beyond a 1:1:1 ratio of the three compounds.

A further aspect of the present application relates to a method of providing the therapeutic composition to maximize the therapeutic efficacy of the composition, and also to overcome mutational escape that might arise when the patient is exposed to either the BET domain ligand alone, or a PROTAC comprising of a BET domain ligand (i.e. JQ1) and an E3 ubiquitin ligase complex ligand (i.e. CRBN-binding ligand). The best of the aforementioned approaches may be combined to overcome multiple mechanisms of potential escape, from increased production of the BET domain protein, to mutation within the BET-domain binding pocket, to mutation outside the BET domain binding pocket, to mutation in one E3 ligase complex machinery, to mutation in the other E3 ligase complex machinery. For example, there are a number of potential ligands that bind the BET domain of BRD4, and two of these (i.e. TPB₁—C₃-L₃, and TPB₂—C₄-L₄) may be used simultaneously with a CRBN binding ligand (i.e. E3ULB₁—C₁-L₁) or a VHL binding ligand (i.e. E3ULB₂—C₂-L₂). Thus, in the general form, the therapeutic composition comprises: a first compound having the chemical structure: E3ULB₁—C₁-L₁, and a second compound having the chemical structure: E3ULB₂—C₂-L₂, and a third compound having the chemical structure: TPB₁—C₃-L₃, and a fourth compound having the chemical structure: TPB₂—C₄-L₄), and where either L₁ or L₂ can form RCBs with L₃ and either L₁ or L₂ can form RCBs L₄. Further, addition of 1.5-fold, 2-fold, 3-fold, 4-fold, 5-fold, 7.5-fold, 10-fold, 15-fold 20-fold, 30-fold, 50-fold, 75-fold, 100-fold or higher concentration or molar amount of either the TPB₁—C3-L₃, or the TPB₂—C₄-L₄ compound, or both, over the E3ULB₁—C₁-L₁ and E3ULB₂—C₂-L₂ compounds may be used to improve the ubiquitination and degradation of a BET domain wild-type and or mutant protein in a biological sample, above and beyond a 1:1:1:1 ratio of the four compounds.

In one aspect of the present application, the therapeutic composition further comprises a third precursor compound having the chemical structure:

E3ULB₂—C₃-L₃,

or a pharmaceutically acceptable salt, enantiomer, stereoisomer, solvate, or polymorph thereof, wherein:
E3ULB₂ is a small molecule E3 ubiquitin ligase binding moiety that binds an E3 ubiquitin ligase, an E3 ubiquitin ligase complex, or subunit thereof that differs in structure from E3ULB,
C₃ is a bond or a connector element, and
L₃ is linker element having a molecular weight of 54 to 420 Daltons and capable of binding to L₂, by two or more reversible bonds that form under physiological conditions, wherein L₂ and L₃ are selected from the group consisting of linker element pairs (1) to (14).

In another aspect of the present application, the therapeutic composition further comprises a third precursor compound having the chemical structure:

TPB₂—C₃-L₃,

or a pharmaceutically acceptable salt, enantiomer, stereoisomer, solvate, or polymorph thereof, wherein:
TPB₂ is a small molecule comprising a BET domain protein binding moiety that differs in structure from TPB,
C₃ is a bond or a connector element, and
L₃ is linker element having a molecular weight of 54 to 420 Daltons and capable of binding to L₁, by two or more reversible covalent bonds that form under physiological conditions, wherein L₃ and L₁ are selected from the group consisting of linker element pairs (1) to (14).

In another aspect of the present application, the the therapeutic composition further comprises a third precursor compound having the chemical structure:

E3ULB₂—C₃-L₃,

or a pharmaceutically acceptable salt, enantiomer, stereoisomer, solvate, or polymorph thereof, and
a fourth precursor compound having the chemical structure:

TPB₂—C₄-L₄,

or a pharmaceutically acceptable salt, enantiomer, stereoisomer, solvate, or polymorph thereof, wherein:
E3ULB₂ is a small molecule E3 ubiquitin ligase binding moiety that binds an E3 ubiquitin ligase, an E3 ubiquitin ligase complex, or subunit thereof that differs in structure from E3ULB,
TPB₂ is a small molecule comprising a BET domain protein binding moiety that differs in structure from TPB,
C₃ and C₄ are bonds or connector elements, and
L₃ and L₄ are linker elements having a molecular weight of 54 to 420 Daltons, with L₁ being capable of binding to L₂ or L₄ but not to L₃, with L₃ being capable of binding to L₂ or L₄ but not L₁, by two or more reversible covalent bonds that form under physiological conditions, wherein L₃ and L₄ are selected from the group consisting of linker element pairs (1) to (13).

A further aspect of the present application relates to a method of treating a BET domain protein-mediated disorder, condition, or disease in a patient. The method includes administering to the patient the therapeutic composition of the present application.

In one embodiment the BET domain protein-mediated disorder is a hematological or solid tissue cancer.

BET inhibitors may be useful in the treatment of cancers including, but not limited to, adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute myeloid leukemia (Dawson et al., Nature 478(7370):529-33 (2011); Mertz et al., Proc. Natl. Acad. Sci. USA 108(40):16669-74 (2011); Zuber et al., Nature 478(7370):524-8 (2011), which are hereby incorporated by reference in their entirety), adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma (Wuet et al. J. Biol. Chem. 288:36094-36105 (2013), which is hereby incorporated by reference in its entirety), aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma (Knoechel et al. Nat. Genet. 46:364-370 (2014); Loosveld et al. Oncotarget 5(10):3168-72 (2014); Reynolds et al. Leukemia 28(9):1819-27 (2014); Roderick et al. Blood 123:1040-1050 (2014), which are hereby incorporated by reference in their entirety), angiomyolipoma, angiosarcoma, astrocytoma, atypical teratoid rhabdoid tumor, B-cell acute lymphoblastic leukemia (Ott et al., Blood 120(14):2843-52 (2012), which is hereby incorporated by reference in its entirety), B-cell chronic lymphocytic leukemia, B-cell prolymphocytic leukemia, B-cell lymphoma (Greenwald et al., Blood 103(4):1475-84 (2004), which is hereby incorporated by reference in its entirety), basal cell carcinoma, biliary tract cancer, bladder cancer, blastoma, bone cancer (Lamoureux et al. Nat. Commun. 5:3511 (2014), which is hereby incorporated by reference in its entirety) Brenner tumor, Brown tumor, Burkitt's lymphoma (Mertz et al., Proc. Natl. Acad. Sci. USA 108(40):16669-74 (2011), which is hereby incorporated by reference in its entirety), breast cancer (Feng et al. Cell Res 24:809-819 (2014); Nagarajan et al. Cell Rep. 8:460-469 (2014); Shi et al. Cancer Cell 25:210-225 (2014), which are hereby incorporated by reference in their entirety), brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma (Chapuy et al. Cancer Cell 24:777-790 (2013); Trabucco et al. Clin. Can. Res. 21(1):113-122 (2015); Ceribelli et al. Proc. Natl. Acad. Sci. USA 111:11365-11370 (2014), which are hereby incorporated by reference in their entirety), dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme (Cheng et al. Clin. Can. Res. 19:1748-1759 (2013); Pastori et al. Epigenetics 9:611-620 (2014), which are hereby incorporated by reference in their entirety), glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma (Lwin et al. J Clin. Invest. 123:4612-4626 (2013), which is hereby incorporated by reference in its entirety), invasive lobular carcinoma, intestinal cancer, kidney cancer, laryngeal cancer, lentigo maligna, lethal midline carcinoma, leukemia, Leydig cell tumor, liposarcoma, lung cancer, lymphangioma, lymphangiosarcoma, lymphoepithelioma, lymphoma, acute lymphocytic leukemia, acute myelogenous leukemia (Mertz et al., Proc. Natl. Acad. Sci. USA 108(40):16669-74 (2011), which is hereby incorporated by reference in its entirety), chronic lymphocytic leukemia, liver cancer, small cell lung cancer, non-small cell lung cancer (Lockwood et al. Proc. Natl. Acad. Sci. USA 109:19408-19413 (2012); Shimamura et al. Clin. Can. Res. 19:6183-6192 (2013), which are hereby incorporated by reference in their entirety) MALT lymphoma, malignant fibrous histiocytoma, malignant peripheral nerve sheath tumor (Baude et al. Nat. Genet. 46:11.54-1155 (2014); Patel et al. Cell Rep. 6:81-92 (2014), which are hereby incorporated by reference in their entirety), malignant triton tumor, mantle cell lymphoma (Moms et al. Leukemia 28:2049-2059 (2014), which is hereby incorporated by reference in its entirety), marginal zone B-cell lymphoma, mast cell leukemia, mediastinal germ cell tumor, medullary carcinoma of the breast, medullary thyroid cancer, medulloblastoma (Bandopadhayay et al. Clin. Can. Res. 20:912-925 (2014); Henssen et al. Oncotarget 4(11):2080-2089 (2013); Long et al. J. Biol. Chem. 289(51):35494-35502 (2014); Tang et al. Nat. Med. 20(7):732-40 (2014); Venataraman et al. Oncotarget 5(9):2355-71 (2014), which are hereby incorporated by reference in their entirety) melanoma (Segura et al. Cancer Res. 72(8):Supplement 1 (2012), which is hereby incorporated by reference in its entirety), meningioma, Merkel cell cancer, mesothelioma, metastatic urothelial carcinoma, mixed Mullerian tumor, mixed lineage leukemia (Dawson et al., Nature 478(7370):529-33 (2011), which is hereby incorporated by reference in its entirety), mucinous tumor, multiple myeloma (Delmore et al., Cell 146(6):904-17 (2010), which is hereby incorporated by reference in its entirety), muscle tissue neoplasm, mycosis fungoides myxoid liposarcoma, myxoma, myxosarcoma, nasopharyngeal carcinoma, neurinoma, neuroblastoma (Puissant et al. Cancer Discov 3:308-323 (2013); Wyce et al. PLoS One 8:e72967 (2014), which are hereby incorporated by reference in their entirety), neurofibroma, neuroma, nodular melanoma, NUT-midline carcinoma (Filippakopoulos et al., Nature 468(7327):1067-73 (2010), which is hereby incorporated by reference in its entirety), ocular cancer, oligoastrocytoma, oligodendroglioma, oncocytoma, optic nerve sheath meningioma, optic nerve tumor, oral cancer, osteosarcoma (Lamoureux et al., Nat. Commun. 5:3511 (2014); Lee et al., Int. J. Cancer 136(9):2055-2064 (2014), which are hereby incorporated by reference in their entirety), ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma (Tolani et al. Oncogene 33:2928-2937 (2014), which is hereby incorporated by reference in its entirety), primary peritoneal cancer, prostate cancer (Asangani et al., Nature 510:278-282 (2014); Cho et al., Cancer Discov. 4:318-333 (2014); Gao et al., PLoS One 8:e63563 (2013): Wyce et al. Oncotarget 4:2419-2429 (2013), which are hereby incorporated by reference in their entirety), pancreatic cancer (Sahai et al. Mol. Cancer Ther. 13:1907-1917 (2014), which is hereby incorporated by reference in its entirety), pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

While it may be possible for compounds of the present application to be administered as the raw chemical, they may also be administered as a pharmaceutical composition. In accordance with an embodiment of the present application, there is provided a pharmaceutical composition including the therapeutic compositions of the present application, or a pharmaceutically acceptable salts or solvates thereof, together with one or more pharmaceutically carriers thereof and optionally one or more other therapeutic ingredients.

The carrier(s) must be “acceptable” in the sense of being compatible with the other ingredients of the formulation and not deleterious to the recipient thereof.

Formulations include those suitable for oral, parenteral (including subcutaneous, intradermal, intramuscular, intravenous, and intraarticular), rectal and topical (including dermal, buccal, sublingual, and intraocular) administration. The most suitable route may depend upon the condition and disorder of the recipient. The formulations may conveniently be presented in unit dosage form and may be prepared by any of the methods well known in the art of pharmacy. Such methods include the step of bringing into association compounds of the present application or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets, or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The therapeutic composition active ingredients may also be presented as a bolus, electuary, or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The pharmaceutical compositions may include a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with the compounds of the present application to insure the stability of the formulation. The composition may contain other additives as needed including, for example, lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to, binders, fillers, disintegrants, lubricants, anti-microbial agents, and coating agents.

Dose ranges for adult humans may vary. The precise amount of the compound administered to a patient will be the responsibility of the attendant physician. However, the dose employed will depend on a number of factors, including the age and sex of the patient, the precise disorder being treated, and its severity.

A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein.

Additional information about pharmaceutical compositions and their formulation is described in Remington: The Science and Practice of Pharmacy, 20^{th} Edition, 2000, which is hereby incorporated by reference in its entirety.

In practicing the methods of the present application, the administering step is carried out to treat a BET domain protein-mediated disorder, condition, or disease in a subject. In one embodiment, a subject having a BET domain protein-mediated disorder, condition, or disease is selected prior to the administering step. Such administration can be carried out systemically or via direct or local administration. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterially, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of the agent will vary depending on the therapeutic agent and the disease to be treated.

The compositions of the present application may be orally administered, for example, with an inert diluent, or with an assimilable edible carrier, or it may be enclosed in hard or soft shell capsules, or it may be compressed into tablets, or they may be incorporated directly with the food of the diet. The therapeutic compositions of the present application may also be administered in a time release manner incorporated within such devices as time-release capsules or nanotubes. Such devices afford flexibility relative to time and dosage. For oral therapeutic administration, the agents of the present application may be incorporated with excipients and used in the form of tablets, capsules, elixirs, suspensions, syrups, and the like. Such compositions and preparations should contain at least 0.1% of the compounds, although lower concentrations may be effective and indeed optimal. The percentage of the compounds in these compositions may, of course, be varied and may conveniently be between about 2% to about 60% of the weight of the unit. The amount of the compounds of the present application in such therapeutically useful compositions is such that a suitable dosage will be obtained.

While the therapeutic composition of the present application are preferably administered orally, they may also be administered parenterally. When the compounds of the present application are administered parenterally, solutions or suspensions of the compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose, and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the therapeutic composition of the present application systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the compositions of the present application can also be achieved using infusion pump devices. Such devices allow continuous infusion of desired compounds avoiding multiple injections and multiple manipulations.

In addition to the formulations described previously, the compositions of the present application may also be formulated as a depot preparation. Such long acting formulations may be formulated with suitable polymeric or hydrophobic materials (for example, as an emulsion in an acceptable oil) or ion exchange resins, or as sparingly soluble derivatives, for example, as a sparingly soluble salt.

A final aspect of the present application relates to a method of treatment including selecting a subject with a BET domain protein mediated disorder, condition, or disease; and administering to the selected subject the therapeutic composition of the present application.

### Considerations for In Vitro Screening of CURE-PRO Molecules Binding to Targets

Two screens, termed “AlphaScreen” and “AlphaLISA” have been developed (sold by Perkin-Elmer) to measure cell signaling, including protein:protein, protein:peptide, protein:small molecule or peptide:peptide interactions. The assays are based on detecting the close proximity of donor beads containing a first molecule or protein that binds to a second molecule or protein on the acceptor beads. Singlet oxygen molecules, generated by high energy irradiation of donor beads, travel over a constrained distance (approx. 200 nm) to acceptor beads. This results in excitation of a cascading series of chemical reactions, ultimately generating a chemiluminescent signal. (Eglen, et al., Curr. Chem. Genomics 1:1-19 (2008), which is hereby incorporated by reference in its entirety).

The donor bead contains phthalocyanine. Excitation of the donor bead by a laser beam at a wavelength of 680 nM allows ambient oxygen to be converted to singlet oxygen. This is a highly amplified reaction since approx. 60,000 singlet oxygen molecules can be generated and travel at least 200 nm in aqueous solution before decay. Consequently, if the donor and acceptor beads are brought within that proximity as a consequence of protein:protein, protein:peptide, or protein:small molecule interactions, energy transfer occurs. Singlet oxygen molecules react with chemicals in the acceptor beads to produce a luminescent response. If the acceptor bead contains Rubrene, as in the AlphaScreen assay, a somewhat broad luminescence is emitted at a wavelength range of 540-680 nm, with detection generally between 540 and 620 nM, and more specifically centered at 570 nm. If the acceptor bead contains Europium, as in the AlphaLISA assay, an intense luminescence is emitted at a wavelength of 615 nm (range 605-625 nm). (Eglen et al., Curr. Chem. Genomics 1:1-19 (2008), which is hereby incorporated by reference in its entirety).

For the purposes of the discussion below, this system will be referred to as linking various proteins, fragments or molecules on donor and acceptor beads. Such linking may be chemical in nature or may be due to tight binding of a tethered ligand, such as if the donor bead is coated with streptavidin and the donor molecule or protein has a biotin attached to it. There are many systems for binding recombinant proteins to beads, including, but not limited to monoclonal antibodies strongly binding highly antigenic epitopes such as V5 tag found on the P and V proteins of the paramyxovirus of simian virus 5 (SV5) with all 14 amino acids (GKPIPNPLLGLDST (SEQ ID NO: 1)), or with a shorter 9-amino acid (IPNPLLGLD (SEQ ID NO:2)) sequence; FLAG-tag, or FLAG octapeptide, or FLAG epitope (DYKDDDDK (SEQ ID NO:3)); Myc-Tag (EQKLISEEDL (SEQ ID NO:4)); and Human influenza hemagglutinin aa 98-106, or HA-tag (YPYDVPDYA (SEQ ID NO:5)). Other ligand systems for binding recombinant proteins to beads include but are not limited to His-Tag or Histidine-6 Tag (HHHHHH (SEQ ID NO:6)); LgBiT to capture HiBiT 11-amino acid tag (VSGWRLFKKIS (SEQ ID NO:7)); GST fusions; and Maltose binding protein (MBP) fusions.

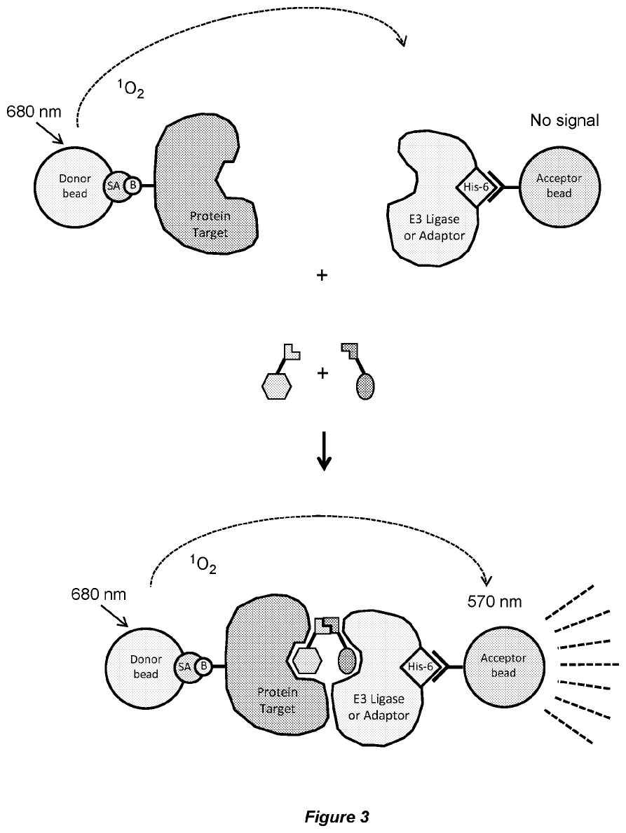

An example of identifying CURE-PRO molecule combinations suitable for the degradation of the BET domain protein target BRD4 is illustrated in FIG. 3. In the initial library screen, the BET domain protein contains a FLAG-tag, and both E3 ligase ligands and BRD4 pharmacophores are synthesized with suitable linkers as described above. For the AlphaScreen assay illustrated in FIG. 3, BET domain protein is expressed in a bacterial or eukaryotic expression system, purified and then appended to a donor bead. In one embodiment, a biotin is chemically appended to the mutant protein at a free amino group (using amine biotinylation reagents NHS-esters and sulfo-NHS-esters; ThermoFisher, Carlsbad, Calif. 92008), such that the protein is appended in multiple orientations. In another embodiment (not illustrated) the protein has the FLAG-tag and the donor bead has anti-FLAG antibody. The E3 ligase, or substrate recognition protein, i.e., the adaptor protein is also expressed in a bacterial or eukaryotic expression system, purified and then appended to an acceptor bead. As above, this may be achieved through biotinylation, or via capture of a tag with an Antibody or other high-affinity interaction (His-6 tag illustrated). The donor and acceptor beads are mixed in 96 or 384 well microtiter plates, each well comprising one or a family of CURE-PRO molecule(s) with BET domain protein target ligands, as well as CURE-PRO molecule(s) with a known pharmacophore for the E3 ligase or adaptor protein. The two different families of CURE-PRO molecules comprise compatible linkers with optional connectors of different length to maximize the chances that a given linker-connector combination will result in the desired quaternary complex comprising the BET domain target protein, two CURE-PRO molecules covalently linked to each other, and the E3 ligase or adaptor protein. Excitation of the donor bead by a laser beam at a wavelength of 680 nM generates singlet oxygen, and if the acceptor bead is in close proximity due to the desired quaternary complex formation, a luminescent signal will be detected at 570 nm.

### Considerations for Assays of CURE-PRO Molecules to Identify Best Combinations for Targeted Degradation of BET Domain Proteins in Cell Lines

Traditionally, protein degradation is detected through Western blots using protein-specific antibodies. However, in some cases, degradation of a given protein (especially those involved in cancer cell growth and signaling) leads to a phenotypic change, such as metabolic activity, compromised cell membrane integrity, cell viability or senescence that may be detected/screened for using 96 or 384 well fluorescent, colorimetric, or luminescent formats. Several of these assays are commercially available, and information below was excerpted in whole or in part from websites describing these products and made available through Promega Corp. (Madison/Fitchburg, Wis., 53711) and Enzo Life Sciences (Farmingdale, N.Y., 11735).
(i) The CellTiter-Blue® Cell Viability Assay, available from Promega Corp., provides a homogeneous, fluorescent method for monitoring cell viability (O'Brien. et al., Eur. J. Biochem. 267:5421-6 (2000), which is hereby incorporated by reference in its entirety). Healthy cells to convert a redox dye (resazurin) into a fluorescent end product (resorufin), whereas nonviable or dead cells rapidly lose the metabolic capacity to reduce the indicator dye, and therefore do not generate a fluorescent signal. The fluorescent signal is subsequently measured with a fluorometer (530-570 nm for excitation and 580-620 nm for emission).
(ii) In the RealTime Glo MT Cell Viability Assay, available from Promega Corp., a non-lytic NanoLuc Luciferase reaction is an example of an in situ assay for determining cell viability. NanoLuc Luciferase and MT Cell Viability Substrate are added to cell culture media, and the substrate is reduced to form a NanoLuc Substrate in healthy cells, which exits the cell and is used rapidly by NanoLuc Luciferase in the media. Only metabolically active cells can reduce the substrate, and light production is proportional to the number of live cells in culture as dead cells are unable to reduce the pro-substrate and therefore do not produce a luminescent signal (Duellman, et al., Assay Drug Dev. Technol. 13(8):456-465 (2015), which is hereby incorporated by reference in its entirety).
(iii) Results obtained using the CellTiter-Glo® Luminescent Cell Viability Assay (Promega Corp.) are discussed in the examples. CellTiter-Glo® Luminescent Cell Viability Assay is a homogeneous method of determining the number of viable cells in culture, in a multiwell format, based on quantitation of the ATP present, an indicator of metabolically active cells. The CellTiter-Glo® Assay generates a rapid luminescent signal that is directly proportional to the amount of ATP present and indicates the number of healthy cells present in the culture well. (Farfan et al., CellNotes 10:2-5(2004), which is hereby incorporated by reference in its entirety).
(iv) The CellTiter-Fluor™ Cell Viability Assay (Promega) is a non-lytic, single-reagent-addition fluorescence assay that measures the relative number of viable cells in a multiwell format. The assay measures the activity of a constitutive protease activity within live cells and therefore serves as a biomarker of cell viability. The live-cell protease activity is restricted to intact viable cells and is measured using a fluorogenic, cell-permeant, peptide substrate (Gly-Phe-AFC) that is cleaved by viable cells to generate a fluorescent signal proportional to the number of living cells. (Niles et al., Anal. Biochem. 366:197-206 (2007), which is hereby incorporated by reference in its entirety).
(v) Normal primary cells proliferate in culture for a limited number of passages prior to undergoing terminal growth arrest and acquiring a senescent phenotype. Senescent cells are characterized by an irreversible G1 growth arrest and are resistant to mitogen-induced proliferation. Senescent cells show common biochemical markers such as expression of an acidic senescence-associated β-galactosidase (SA-β-gal) activity. The 96-well Cellular senescence assay kit, available from Enzo Life Sciences, determines the cellular senescence by measuring SA-β-gal activity using a fluorometric substrate (Dimri Proc Natl Acad Sci USA. 92(20):9363-7 (1995)).

An alternative approach is to monitor protein degradation kinetics directly using an engineered target protein and a luminescent assay (Riching et al., “Quantitative Live-Cell Kinetic Degradation and Mechanistic Profiling of PROTAC Mode of Action,” ACS Chem. Biol. 13(9):2758-2770 (2018), which is hereby incorporated by reference in its entirety). Briefly, the authors used CRISPR/Cas9 genome editing to append an 11 amino acid peptide (HiBit) to either the N or C-terminus of the target protein. The HiBit peptide is small enough that it does not interfere with protein function, yet it has high affinity to an 18 kD LgBiT protein, forming the luminescent luciferase termed NanoBit. (Schwinn et al., “CRISPR-Mediated Tagging of Endogenous Proteins with a Luminescent Peptide,” ACS Chem. Biol. 13(2):467-474 (2018), which is hereby incorporated by reference in its entirety). In these experiments, LgBiT is added endogenously on a plasmid construct and the efficacy of various PROTAC drugs in directing degradation of the target protein (comprising of the HiBit peptide) may be monitored continuously over an extended time-period. The same approach may be applied to determine the relative potency of different CURE-PRO molecule combinations to identify optimal linkers and connector lengths for a given target-E3 ligase machinery combination.

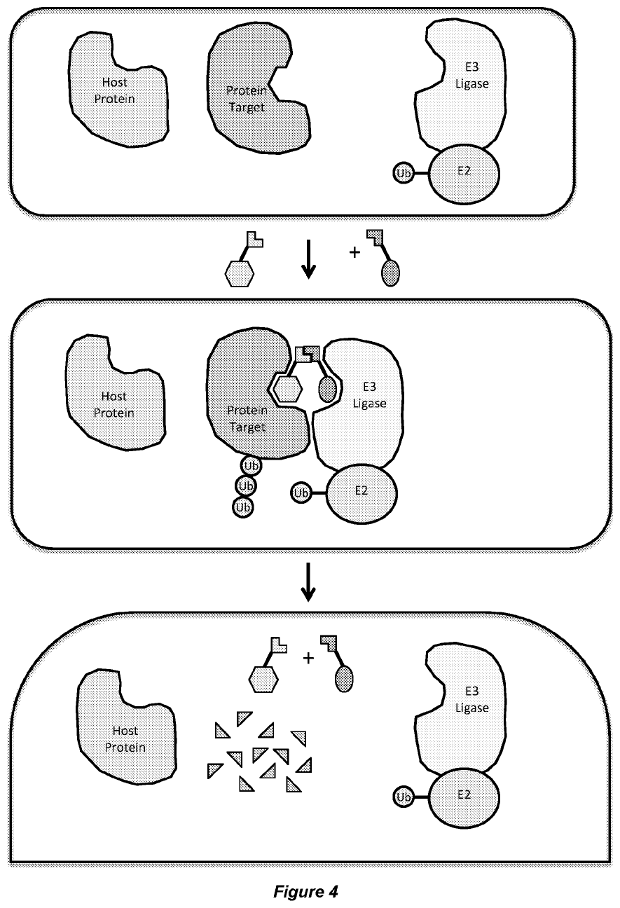

Generic screening of native protein target degradation in the presence of two CURE-PRO molecules binding the target protein and an E3 ligase is illustrated in FIG. 4. The screen identifies hits resulting in a phenotypic change of the biologically harvested cells, cell line, or organoid that is scored by one of the fluorescent, colorimetric, or luminescent assays as described above, or by other assays known to those skilled in the art. However, many molecules may be cytotoxic and can lead to a given phenotypic change that is unrelated to proteasomal degradation of the desired target. While a confirmatory Western blot would reveal loss of the target protein, this may also occur from activation of proteases. Thus, to validate that the CURE-PRO molecules are responsible for the directed ubiquitination and subsequent degradation of the desired protein, the following controls would need to be included: (i) Addition of both CURE-PRO molecules results in the scored phenotype, (ii) Addition of either one or the other CURE-PRO molecules does not result in the scored phenotype, (iii) Pretreatment of cell with an excess of the E3 ligand (lacking a linker) blocks or squelches the scored phenotype, and (iv) Pretreatment of cell with a 26S proteasome inhibitor blocks or squelches the scored phenotype. Known 26S proteasome inhibitors include but are not limited to Carfilzomib (PR-171), Bortezomib (PS-341), MG132, and VR23 (Raina et al., Proc Natl Acad Sci USA. 113(26):7124-9 (2016); Adams J. Cancer Cell. 5(5):417-21 (2004); and Pundir et al., Cancer Res. 75(19):4164-75 (2015), which is hereby incorporated by reference in its entirety).

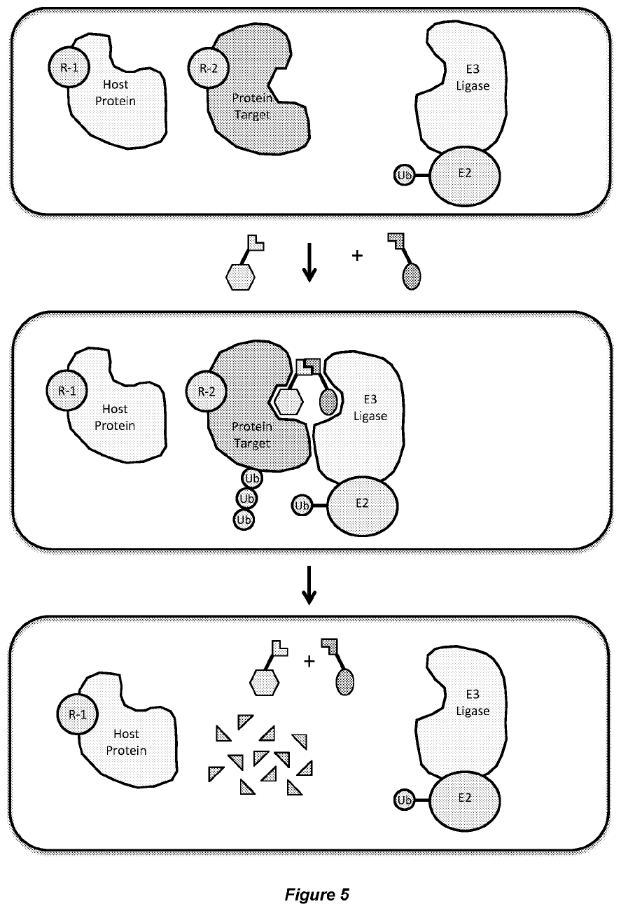

To facilitate screening for CURE-PRO molecules that accelerate target protein degradation, reporter groups that allow for a fluorescent, colorimetric, or luminescent assay may be appended to the protein, such that its destruction also results in destruction or loss of the reporter group (See FIG. 5). In this example, cells are engineered to contain a first reporter group, such as GFP (green fluorescent protein; emission wavelength 509 nm), which may be fused to a host protein, while a second reporter group, such as YFP (yellow fluorescent protein, such as Citrine; emission wavelength 529 nm) may be fused to the desired protein target. Addition of two CURE-PRO molecules, the first CURE-PRO molecule comprising a known pharmacophore element (illustrated as the hexagonal shaped element) that binds the desired protein target, said molecule also comprising a linker element (illustrated as the light L shaped element) capable of making a reversible covalent linkage to a partner linker element (illustrated as the dark L shaped element) of the second CURE-PRO molecule which comprises a known E3 ligase or adapter protein ligand (illustrated as the oval shaped element) to the engineered cells will result in targeted ubiquitination and selective degradation of the target protein. This may be detected by observing a decreased ratio of YFP/GFP signal, e.g. decreased ratio of 529 mm/509 nm signal. A pharmacophore that increased overall protein degradation would result in decrease of both YFP and GFP signal without a significant change in their ratio, and thus would be distinguished from a true hit. Likewise, a pharmacophore that bound to the GFP would most likely also bind the YFP, and would also result in decreasing both signals, further it would be distinguished on control cells comprising just the GFP and YFP proteins. To avoid false-positives from pharmacophores that would either reduce the expression of the desired protein target, or increase the expression of the host protein, positive hits are validated by demonstrating that pretreatment with a 26S proteasome inhibitor or the E3 ligase ligand lacking a linker element squelches degradation and reverts the ratio of YFP/GFP signal to that of untreated cells. Additional reporter groups include TagBFP (blue), mCerulean3 (cyan), mCitrine/mVenus (green-yellow), tdTomato (orange), mCherry and mApple (red), and mKate2 and mNeptune (far-red), which may be used for multiplexed labeling of several targets (Crivat and Taraska Trends Biotechnol. 30(1):8-16 (2012), which is hereby incorporated by reference in its entirety).

As an alternative to using fluorescent proteins, a number of commercially available kits allow for using a protein to catalyze the covalent auto-attachment of a fluorophore within a cell. The 20-kDa DNA repair protein human O⁶-alkylguanine-DNA alkyltransferase (AGT; available as SNAP tag from New England Biolabs, Ipswich, Mass.) has been engineered to catalyze the attachment of a fluorescent membrane-permeable O⁶-alkylguanine substrate. When added to cells expressing proteins with genetic in-frame fusion of AGT, the desired protein is specifically labeled by the cell-permeable fluorescent substrate (Juillerat et al., Chem. Biol. 10(4):313-7 (2003), which is hereby incorporated by reference in its entirety). Cell permeable O⁶-alkylguanine substrates include SNAP-Cell 505-Star and SNAP-Cell fluorescein (emission wavelength 532 nm); SNAP-Cell Oregon Green (emission wavelength 514 nm); SNAP-Cell TMR-Star (emission wavelength 580 nm); SNAP-Cell 430 (emission wavelengths 44 & 484 nm); and SNAP-Cell 647-SiR (emission wavelength 661 nm). An engineered variant of this enzyme has also been developed and reacts specifically with O⁶-benzylcytosine substrates (available as CLIP tag, also from New England Biolabs, Ipswich, Mass.) (Gautier et al., Chem. Biol. 15(2):128-36 (2008), which is hereby incorporated by reference in its entirety). An advantage of using these two orthogonal labeling systems is the ability to not only provide two different labels for the control (host) protein and the targeted protein, but also to provide a pulse of label prior to drug treatment, and then add a second label or blocking substrate during drug treatment, such that newly synthesized target protein either has a second label or no additional label. This approach easily enables distinction between a CURE-PRO pharmacophore that directs targeted degradation of the desired protein from a CURE-PRO pharmacophore that directs destruction of an upstream transcription factor, resulting in decreased synthesis of the desired protein. In the first case (specific degradation), the ratio of the desired protein pulse/chase label will remain the same, and the ratio of desired protein pulse/host protein will decrease, while in the second case (decreased synthesis), the ratio of the desired protein pulse/chase label will increase, and the ratio of desired protein pulse/host protein will remain the same or only decrease slightly.

Additionally, the bacterial enzyme haloalkane dehalogenase (available as Halo tag, Promega, Madison, Wis.) has been engineered to work as a self-labeling fusion tag (Los and Wood Methods Mol Biol. 356:195-208 (2007), which is hereby incorporated by reference in its entirety). Similar to the SNAP-tag system, the Halo-tag enzyme has been engineered to covalently react with a halogenated alkane chain. Cell permeable substrates include HaloTag TMR ligand (emission wavelength 585 nm); HaloTag Oregon Green ligand (emission wavelength 516 nm); HaloTag diAcFam ligand (emission wavelength 526 nm); and HaloTag Coumarin Ligand (emission wavelength 434 nm). Although there is just one version of the HaloTag enzyme, it may be used in conjunction with the SNAP-tag or Clip-Tag system, or with cells already containing a fluorescently labeled host (control) protein. Further, since multiple fluorescent labels are available, the same pulse-chase labeling approach described above. Another advantage of using the HaloTag fusion system is it enables use of a PROTAC comprising a halogenated alkane tail fused to an E3 ligase ligand (VHL) to rapidly test for the desired biological phenotype when targeting destruction of the fusion protein (Buckley et al., ACS Chem. Biol. 10(8):1831-7 (2015), which is hereby incorporated by reference in its entirety). Thus, even in the absence of a known pharmacophore or ligand to the desired protein, the HaloTag system provides a rapid proof of principle that degradation of the target protein is biologically relevant in the disease in question, and that identifying pharmacophores to that target protein is a worthwhile endeavor.

Alternative protein labeling systems to monitor protein degradation include but are not limited to (i) adding a genetically encoded tag comprising of a tetracysteine binding motif (FLNCCPGCCMEP (SEQ ID NO:8)aa) and labeling with biarsenical dyes FLAsH-EDT₂ and/or ReAsH-EDT₂ that become fluorescent upon reacting with the tetracysteine binding motif (Crivat and Taraska Trends Biotechnol. 30(1):8-16 (2012), which is hereby incorporated by reference in its entirety); (ii) Using CRISPR/Cas9 gene editing to append an 11 aa “HiBiT-tag”, and after drug exposure and cell lysis, adding a detection reagent containing the complementing polypeptide LgBiT, which spontaneously interacts with the HiBiT tag to reconstitute the bright, luminescent NanoBiT enzyme (Oh-Hashi et al., Biochem. Biophys. Rep. 12:40-45 (2017), which is hereby incorporated by reference in its entirety); and (iii) PathHunter technology (available through DiscoverX, now Eurofins), which incorporates an adaptation of Enzyme Fragment Complementation (EFC) in a novel, cell-based assay format to detect protein degradation, based on the use of two genetically-engineered β-galactosidase (β-gal) fragments: a large protein fragment (Enzyme Acceptor, EA) and a small peptide fragment (Enzyme Donor, ED) that is genetically fused to the desired target protein; wherein the enzyme fragments combine to form active p-gal enzyme that hydrolyzes a chemiluminescent substrate (Zhao et al., Assay Drug Dev. Technol. 1(6):823-33 (2003), which is hereby incorporated by reference in its entirety). These protein labeling systems have the advantage of appending an extremely small peptide (12, 11, or 38 amino acid residues, respectively), and thus would be predicted to minimally influence the conformation, stability, or activity of the desired native protein. Further, they may be used in conjunction with the other protein labeling systems described above to achieve a two-color assay system if needed. In the examples provided below for identifying CURE-PRO molecules to direct degradation of desired target proteins, a two-reporter system is described, however the screens are also amenable to using just a single reporter system and the appropriate control assays. For example, when identifying CURE-PRO molecules that selectively destroy a mutant protein, but leave wild-type protein intact, the same 11 aa “HiBiT-tag” may be appended to the mutant protein in a first cell line comprising only mutant protein, as well as in a second cell line with both mutant and wild-type protein, while appended to wild-type protein in a third cell line comprising only wild-type protein. Pharmacophores would be screened on all three cell lines, with winning pharmacophores causing significant reduction in the reporter group in the first two, but not the third cell line.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The following Examples are presented to illustrate various aspects of the present application, but are not intended to limit the scope of the claims.

### EXAMPLES

### Materials and Methods

Cell Culture

All cell lines were purchased from ATCC or DSMZ and grown at 37° C. with 5% CO₂. Human HeLa cells were cultured in complete growth medium (Dulbecco's modified Eagle's medium (DMEM) supplemented with 10% FBS); MCF7 cells were cultured in complete growth medium (Dulbecco's modified Eagle's medium (DMEM) supplemented with 10% FBS and 0.01 mg/ml human recombinant insulin (Sigma Aldrich); Namalwa and MOLM-13 cells were cultured in complete growth medium (RPMI 1640 medium supplemented with 10% FBS); MV-4-11 were cultured in complete growth medium (Iscove's Modified Dulbecco's medium, supplemented with 10% FBS); HCT116 cells were cultured in complete growth medium (McCoy's 5a Medium supplemented with 10% FBS). For cellular degradation of BRD4 studies, cells were seeded in a 12-well plate at 70-80% confluency, allowed to attach overnight, and incubated with the indicated compounds for the indicated time. When indicated, a 15-minute pretreatment with 10 μM pomalidomide, 10 μM VHL298, 10 μM Nutlin3a, 1 μM MG-132 or 1 μM Carfilzomib was performed before the addition of CURE-PROs. For washout studies, after CURE-PRO treatment, media was aspirated and incubated with complete medium for the indicated time before lysis.

Antibodies

Anti-BRD4 (13440, 1:1,000 dilution for Western Blot and 1:25 dilution for ProteinSimple); Anti-BRD2 (5848 1:1,000 dilution for Western Blot and 1:25 dilution for ProteinSimple); β-Actin (3700, 1:2,000 dilution for Western Blot), β-Actin (4970, 1:50 dilution for ProteinSimple) anti-mouse IgG-HRP, and anti-rabbit IgG-HRP antibodies were purchased Cell Signaling Technology. Anti-GAPDH (600-401-A33, 1:100 dilution for ProteinSimple) was purchased from Rockland Immunochemicals, Inc. Anti-BRD3 (sc-81202, 1:200 dilution for Western Blot) was purchased from Santa Cruz Biotechnology.

Western Blotting

HeLa, MCF7, or HCT116 cells (2.5-3×10⁶) were treated for 24 hours with the indicated compounds solubilized in DMSO. The cells were washed in ice-cold PBS and were then lysed in RIPA lysis buffer (150 mM NaCl, 5 mM EDTA, 1% NP-40, 0.5% sodium deoxycholate, 0.1% SDS, 50 mM Tris, pH 8.0.) with Roche protease inhibitor complete cocktail and phosphatase inhibitors (10 mM sodium fluoride, 10 mM sodium pyrophosphate, 1 mM sodium orthovanadate and 20 mM β-glycerophosphate). The total protein concentrations were determined by Bradford Protein Assay and 10-20 μg of protein was loaded onto 4-15% Tris-Glycine gradient gels (Biorad). After standard gel electrophoresis, the separated proteins were transferred to PVDF membrane by wet transfer. The immunoblots were then blocked for 1 hour in 5% skim milk in TBST or 5% BSA in TBST, according to manufacturer's instructions, before an overnight incubation at 4° C. with indicated antibodies and membranes. Membranes were then incubated with the appropriate horseradish peroxidase-conjugated secondary antibodies (1:5,000 dilution) for 1 h at room temperature and the bands were visualized using the Clarity Max Western ECL Substrate (Biorad) and the ChemiDoc Imaging System (Biorad). Signal was detected with ECL Western Blotting Substrate (Pierce) and X-ray film processed with a Konica SRX-101 X-ray film processor or captured by Bio-Rad's Chemidoc Imaging system.

WES, ProteinSimple

WES Simple analysis was performed on WES system (ProteinSimple-Biotechne) according to the manufacturer's instructions. Total protein concentrations of cell lysates were determined by the Pierce BCA kit (Thermo Fisher). 3 μL of 0.3 μg/μL of the protein lysate was loaded onto a 12- to 230-kDa WES assay plate (ProteinSimple) where 300 nL sample was withdrawn through a capillary, subjected to electrophoretic separation of proteins by size, and followed by the simultaneous, HRP-based detection of proteins using the Anti-Rabbit Detection Module (Proteinsimple: #DM-001). The electropherograms were checked then the automatic peak detection was manually corrected if it was required.

Analysis of Cellular Viability by CellTiter-Glo® 2.0 Cell Viability Assay

CellTiter-Glo® 2.0 Cell Viability Assay (Promega) was carried out following the manufacturer's recommendations. Cells were seeded at a density of 1000 cells/well in a white 96 well plates (Corning, #3917) in a total volume of 100 μl with respective monomers, combinations of BRD ligands together with E3 ligase ligands or vehicle control treatment. After a 72 h incubation, 100 ul of the CellTiter-Glo® substrate was added per well and luminescence was read on a Spectramax M5 (Molecular Devices). Dose-response curves were generated using Graphpad Prism software.

Analysis of Apoptotic Cells by Caspase-Glo® Assay

Caspase-glo® assay (Promega) was conducted following the manufacturer's recommendations. Cells were seeded at a density of 5000 cells/well in a white 96 well plates (Corning, #3917) in a total volume of 100 μl with respective monomers, combinations of BRD ligands together with E3 ligase ligands or vehicle control treatment. After a 24 h incubation, 100 μl of the CellTiter-Glo® substrate was added per well and luminescence was read on a Spectramax M5 (Molecular Devices).

RT-PCR

The suppression of the expression of the downstream c-MYC target, SLC19A1, was measured by RT-PCR using Power SYBR© Green Cells-to-Ct™ Kit (Life Technologies). Adherent cells were plated on 96 well plates at 5,000 cells per well were treated for 24h with monomer compounds and compounds capable of reversible interactions (1 nM-100 μM). Cells were subsequently washed in ice-cold PBS processed according to the manufacturer's instructions. Quantitative real-time PCR was performed using a ViiA™ 7 Real-time PCR System (Applied Biosystems, Foster City, Calif., USA). GAPDH and ACTB served as internal controls. The primers used were (5′-3′): GAPDH-F: AGCCACATCGCTCAGACAC (SEQ ID NO:9), GAPDH-R: GCCCAATACGACCAAATCC (SEQ ID NO:10), ACTB —F: CCAACCGCGAGAAGATGA (SEQ ID NO: 11), ACTB -R: CCAGAGGCGTACAGGGATAG (SEQ ID NO: 12), SLC19A₁-F: ATGGCCCCCAAGAAGTAGAT (SEQ ID NO:13), SLC19A₁-R: GTCAACACGTTCTTTGCCAC (SEQ ID NO: 14).

Relative Binding Affinities of Boronic Acid Linkers with Diols and Other Binding Partners

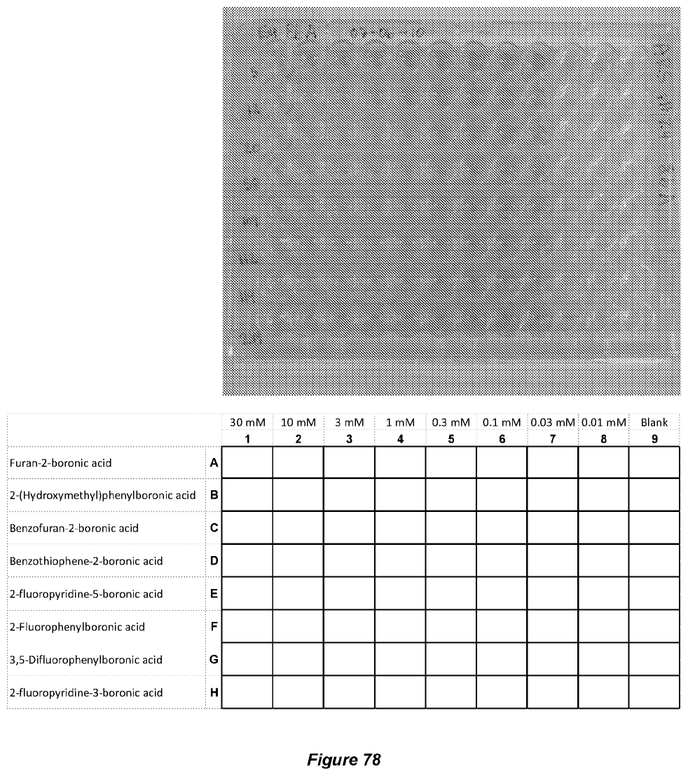

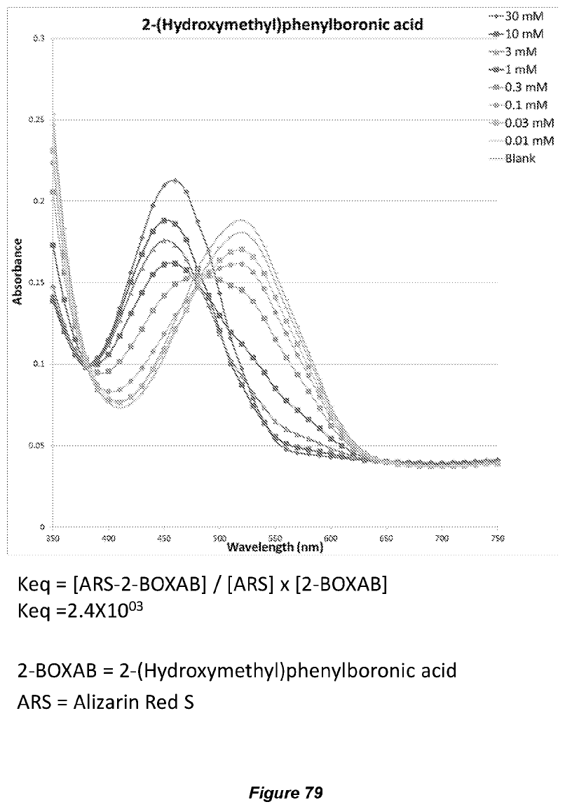

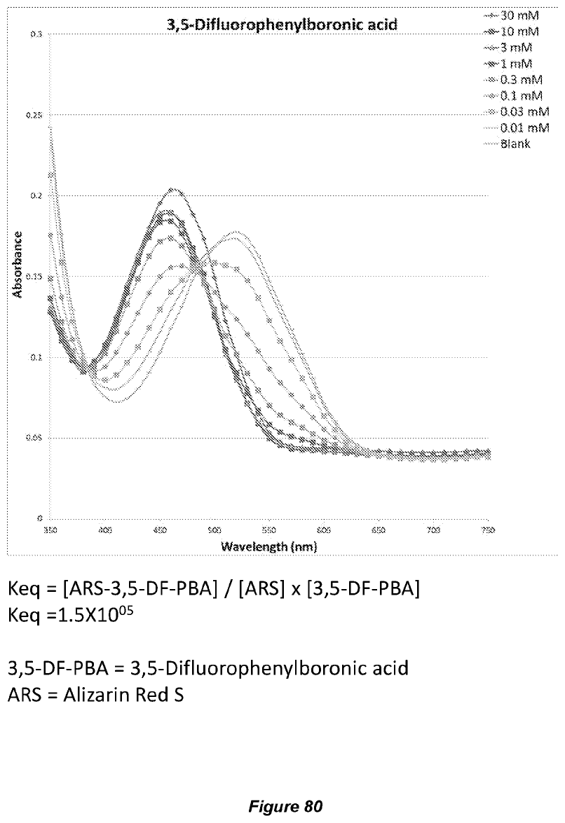

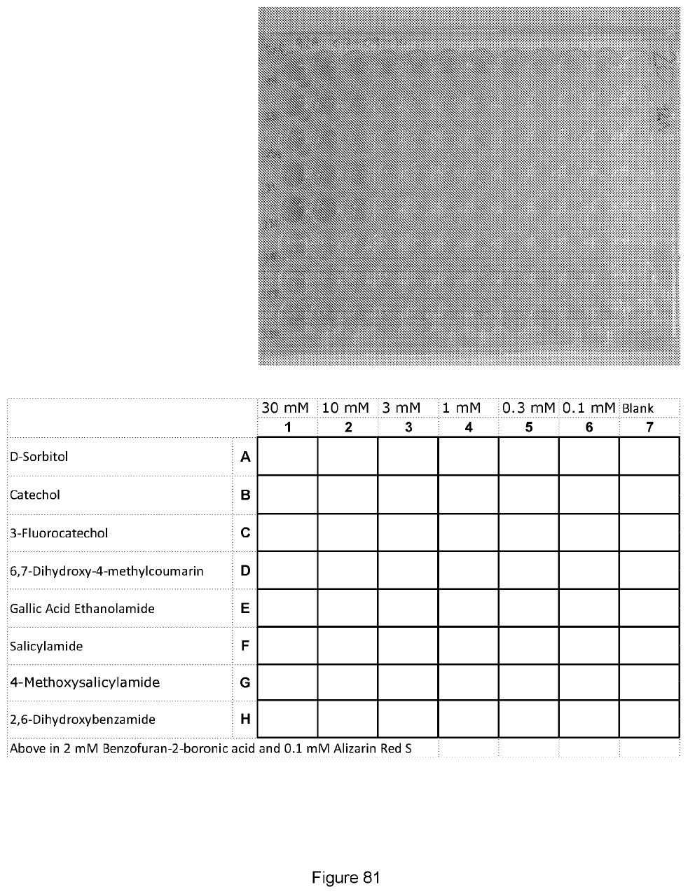

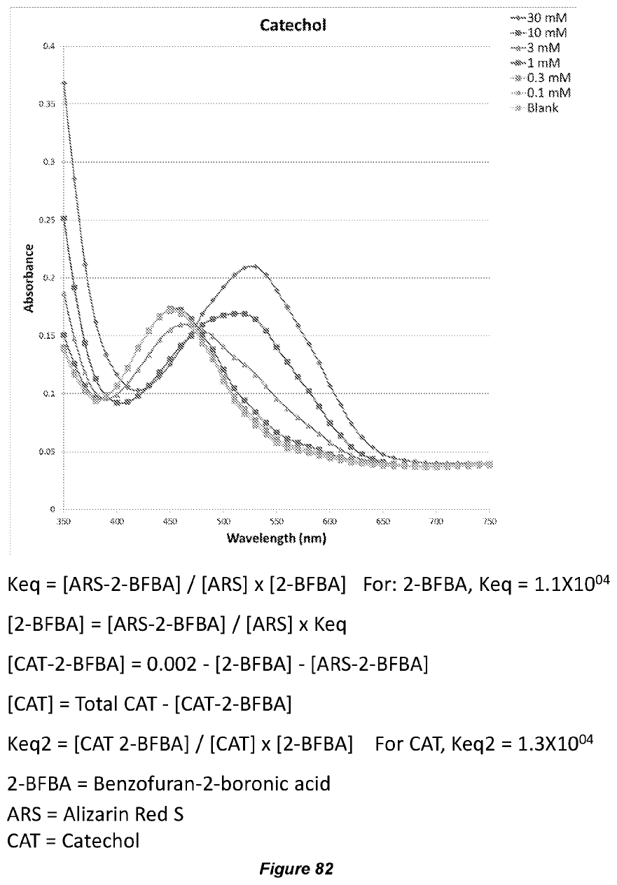

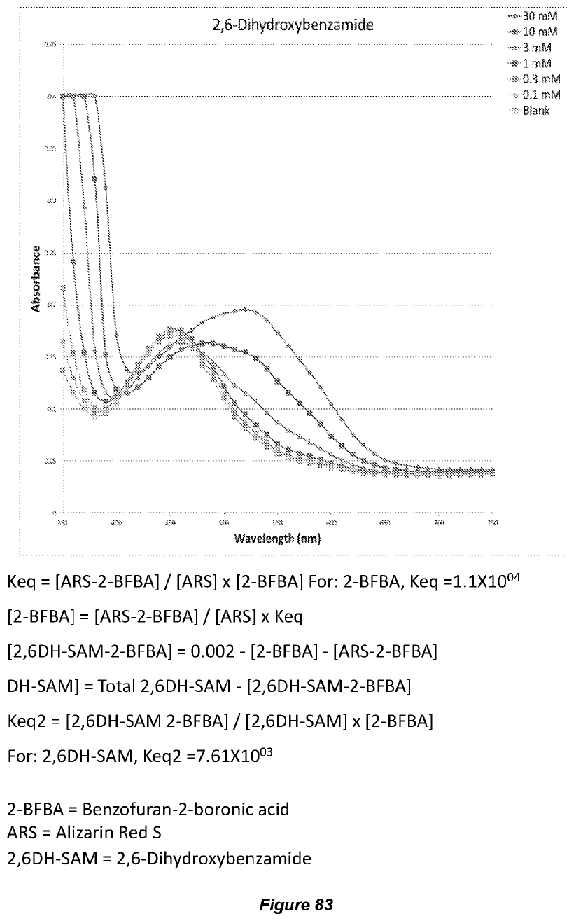

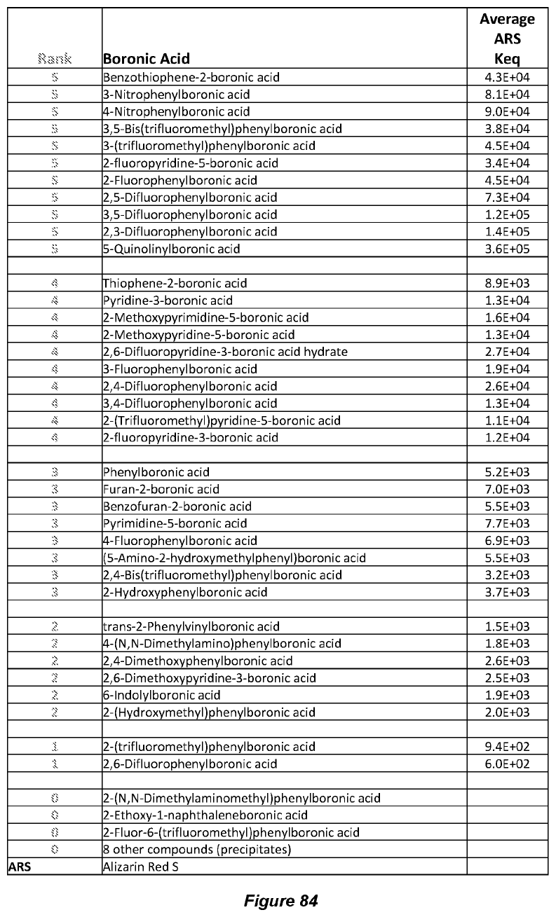

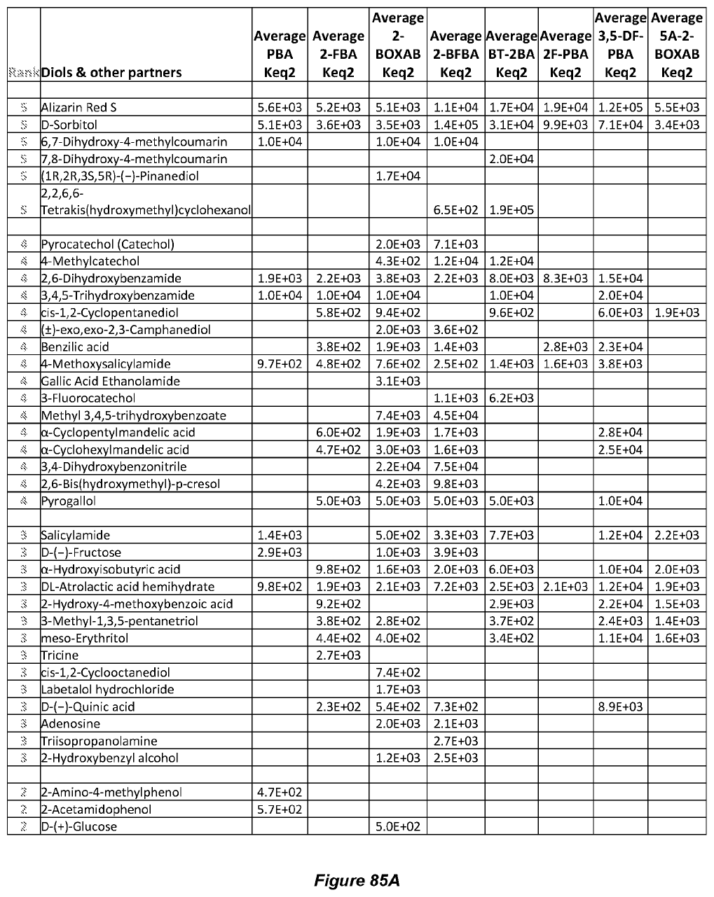

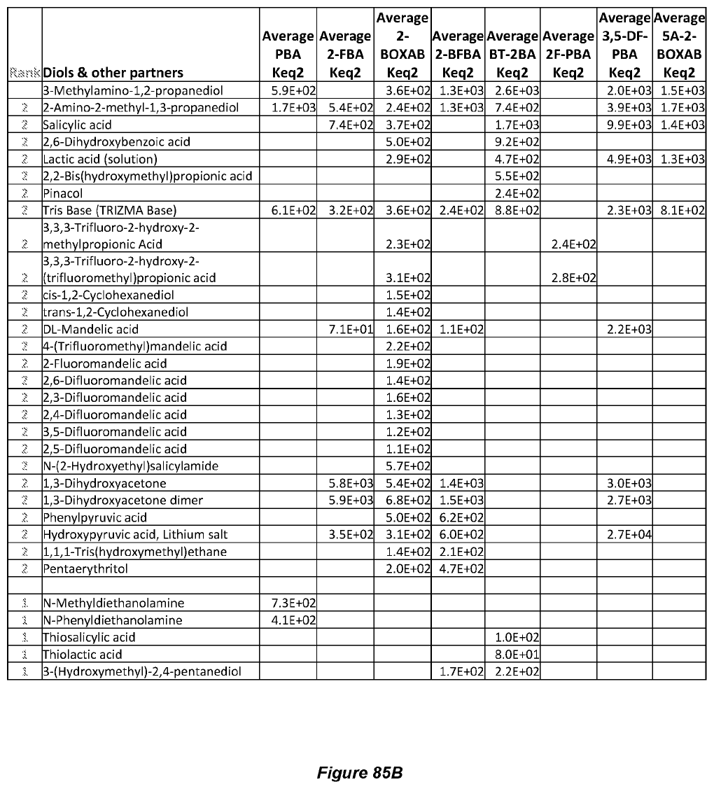

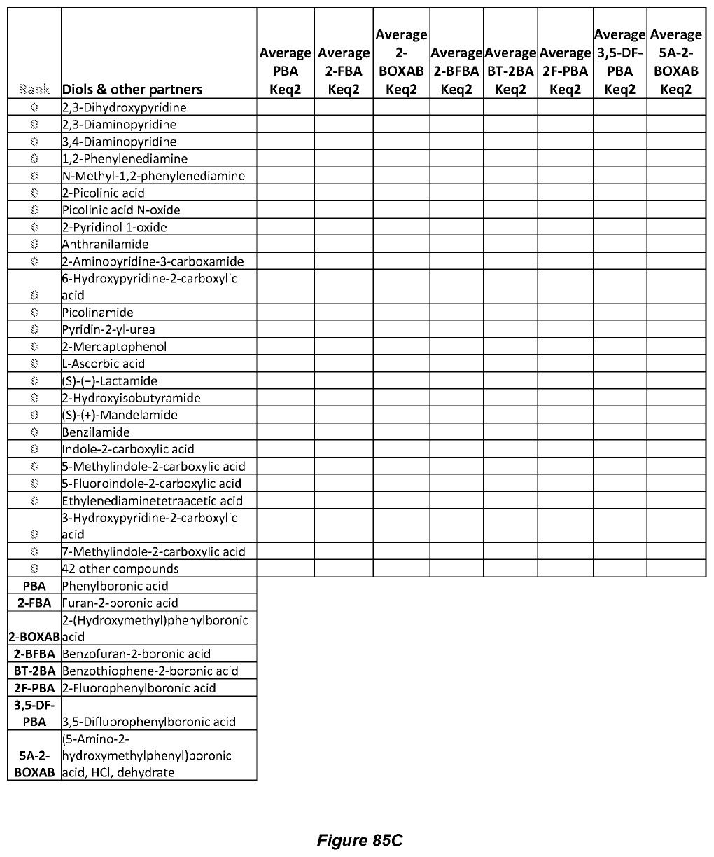

Potential linker moieties were tested for relative binding affinities to each other using the Alizarin Red optical reporter system as described by Springsteen and Wang (Springsteen G. & Wang B., Chem. Commun. (Camb). (17):1608-1609 (2001), which is hereby incorporated by reference in its entirety). Briefly, chemicals were dissolved in 100% DMSO at 100 mM concentrations. Serial dilutions (from 30 mM to 0.01 mM) of the boronic acid was made into 0.1 mM Alizarin Red S. (ARS) in 0.1 M phosphate buffer, pH 7.4, and absorbance determined from 350 to 650 nM, and values at 450 and 540 or 550 nM used to calculate the relative binding affinities of aromatic boronic acids (ABA) to ARS, using the formula K_{eq}=[ARS−ABA]/[ARS]×[ABA]. At higher concentrations of ABA, the ARS turned yellow. For the diols, alpha-hydroxy carboxylic acids, alpha-hydroxyketones and other partners to a variety of boronic acids, 2 mM of the ABA was mixed with 0.1 mM ARS in 0.1 M phosphate buffer, pH 7.4, and then serial dilutions (from 30 mM to 0.1 mM) of the diol etc. were made with absorbance determined as above. For calculating the relative affinity, i.e., K_{eq}2, the following formulas were used (with the example of CAT representing the aromatic cis-diol catechol): K_{eq}=[ARS−ABA]/[ARS]×[ABA]. Therefore; [ABA]=[ARS−ABA]/[ARS]×K_{eq} and [CAT]=Total CAT−[CAT−ABA] and K_{eq}2=[CAT−ABA]/[CAT]×[ABA]. In these experiments, the ABA was in 20-fold excess over ARS, so it turned completely yellow, but then the diols were added at an even higher concentration, where they compete the ABA away from ARS, so the ARS turned back to red. Examples of such experiments and calculation of the K_{eq} and K_{eq}2 are shown in FIGS. 78 through 80 and FIGS. 81 through 83, respectively. The calculated K_{eq} and K_{eq}2 would vary at different concentrations, and across different experiments. The average calculated K_{eq} for various aromatic boronic acids in the Alizarin Red optical reporter system is listed in FIG. 84. The average calculated K_{eq}2 for various diols, α-hydroxy carboxylic acids, α-hydroxyketones and other partners to a variety of boronic acids (phenylboronic acid, furan-2-boronic acid, 2-(hydroxymethyl)phenylboronic acid, benzofuran-2-boronic acid, benzothiophene-2-boronic acid, 2-fluorophenylboronic acid, 3,5-difluorophenylboronic acid, and (5-amino-2-hydroxymethylphenyl)boronic acid, HCl, dehydrate) in the Alizarin Red optical reporter system is listed in FIG. 85A-C. The relative K_{eq} among different boronic acid compounds listed in FIG. 84—arbitrarily ranked from 1-5 was essentially the same. Likewise, the relative K_{eq} among different diols, alpha-hydroxy carboxylic acids, alpha-hydroxyketones and other partners listed in FIG. 85A-C, also arbitrarily ranked from 1-5 was essentially the same among each group of compounds. However, the K_{eq} for a “Rank 3” aromatic boronic acid (i.e., phenylboronic acid) is generally higher than the K_{eq}2 for a “Rank 3” diol, alpha-hydroxy carboxylic acid, alpha-hydroxyketone, or other partner, D-(−)-fructose as an example.

### Example 1—Chemical Synthesis of CURE-PRO Compounds

### Materials and Methods

All commercially available materials were used as received unless otherwise indicated. VH032 was purchased from Tocris and rel-(4R,5S)-4,5-bis(4-chlorophenyl)-2-(2-isopropoxy-4-methoxyphenyl)-4,5-dihydro-1H-imidazole was purchased from ChemScene. 4-(hydroxydimethylsilyl)benzoic acid (WO2009020589 to Grimm et al., which is hereby incorporated by reference in its entirety), 3-(hydroxydimethylsilyl)benzoic acid (WO2009020589 to Grimm et al., which is hereby incorporated by reference in its entirety), tert-butyl (2-(2-oxopiperazin-1-yl)ethyl)carbamate (WO2017025868 to Ninkovic et al., which is hereby incorporated by reference in its entirety) and [2-(2,6-dioxo-3-piperidyl)-1,3-dioxo-isoindolin-4-yl]methylammonium chloride (Jacques, et al., PNAS, 112, E1471-E1479 (2015), which is hereby incorporated by reference in its entirety), were synthesized according to procedures known in literature. All reactions were carried out under an atmosphere of argon in oven dried round bottom flask with magnetic stirring. Reactions were monitored by UPLC (ACQUITY, Waters). HPLC purifications were performed using a Waters AutoPure HPLC/MS system equipped with XBridge OBD prep C18, 5 μm (19×150 mm) column and SQD2 mass spectrometer. All NMR spectra were recorded on Bruker DRX-500 spectrometer (500 MHz for ¹H and 125 MHz for ¹³C). Chemical shifts δ are reported in ppm, with the residual solvent resonance as internal standard. NMR data are reported as following: chemical shift (multiplicity s=singlet; d=doublet; t=triplet; q=quartet; m=multiplet; br=broad, coupling constant in Hz, and integration).

### Synthesis of CRBN Targets

### Synthesis of Common Intermediate 5

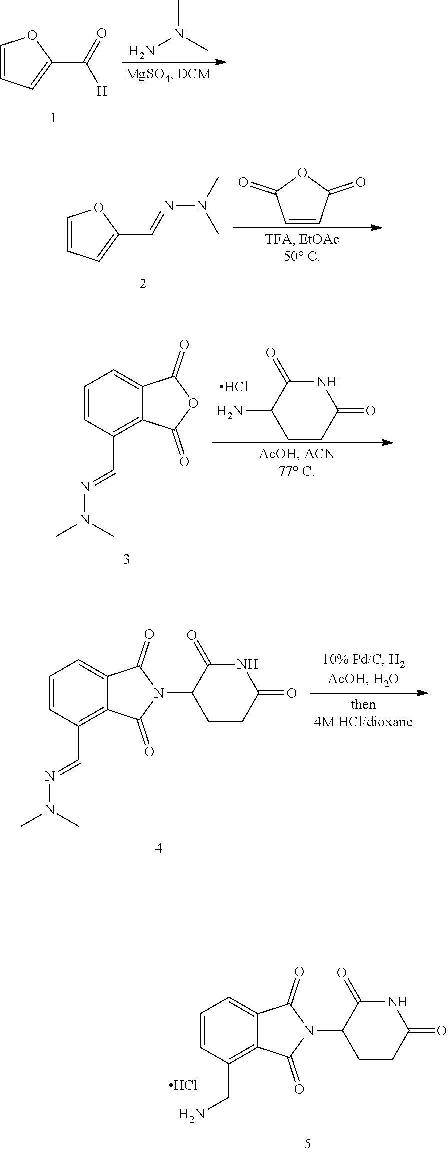

The synthetic approach to common intermediate 5 can be found in Jacques, et al., Proc. Natl. Acad. Sci. 112(12), E1471-E1479 (2015), and U.S. Patent Application Publication No. 2006270707 to Zeldis et al., which are hereby incorporated by reference in their entirety.

### (E)-2-(furan-2-ylmethylene)-1,1-dimethylhydrazine (2)

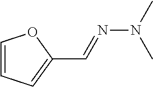

To a stirred solution of furan-2-carbaldehyde 1 (10 g, 104 mmol) in dry dichloromethane (100 mL) at ambient temperature was added magnesium sulfate (25.5 g, 201 mmol) and 1,1-dimethylhydrazine (8.13 g 135 mmol) under an atmosphere of nitrogen. The resulting solution was stirred for 18 h, then concentrated under reduced pressure and further dried under vacuum to give (E)-2-(furan-2-ylmethylene)-1,1-dimethylhydrazine, 2 (14.3 g, 99.9%) as a brown liquid, which was taken on without further purification

### (E)-4-((2,2-dimethylhydrazineylidene)methyl)isobenzofuran-1,3-dione (3)

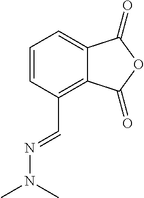 [SMILES: CN(C)/N=C/c1cccc2c1C(=O)OC2=O]

To a stirred solution of (E)-2-(furan-2-ylmethylene)-1,1-dimethylhydrazine, 2
(14.3 g, 104 mmol) in ethyl acetate (100 mL) was added a solution of maleic anhydride (13.3 g, 135 mmol) in ethyl acetate (50 mL) over a period of 10 min. Trifluoroacetic acid (0.4 mL 5.18 mmol) was then added, and the resulting solution was heated to 50° C. and stirred for 4 h, then cooled to ambient temperature and placed in a cold room overnight. The precipitated solid was filtered, washed with ice-cold ethyl acetate and petroleum ether, then dried under reduced pressure to provide (E)-4-((2,2-dimethylhydrazineylidene)methyl)isobenzofuran-1,3-dione, 3 (16.2 g, 71.6%) as a yellow solid, which was taken on without further purification.

### (E)-4-((2,2-dimethylhydrazineylidene)methyl)-2-(2,6-dioxopiperidin-3-yl)isoindoline-1,3-dione (4)

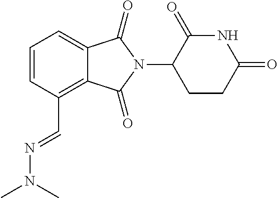 [SMILES: CN(C)/N=C/c1cccc2c1C(=O)N(C1CCC(=O)NC1=O)C2=O]

To a stirred solution of (E)-4-((2,2-dimethylhydrazineylidene)methyl)isobenzofuran-1,3-dione, 3 (16.2 g, 74.2 mmol) in dry acetonitrile (124 mL) at ambient temperature was added imidazole (40.4 g, 59.4 mmol) and 3-aminopiperidine-2-6-dione, hydrochloride (10.3 g, 51.2 mmol) under an atmosphere of nitrogen. Acetic acid (36 mL) was then added and the flask was fitted with Dean-Stark apparatus. The solution was heated at 77° C. for 2 h, during which time a yellow precipitate formed. After 2 h, the reaction was cooled to ambient temperature, diluted with water, and then filtered. The solid material was washed with ice water and petroleum ether, then dried under high vacuum to afford (E)-4-((2,2-dimethylhydrazineylidene)methyl)-2-(2,6-dioxopiperidin-3-yl)isoindoline-1,3-dione, 4 (18.3 g, 75%) as a yellow solid.

### 4-(aminomethyl)-2-(2,6-dioxopiperidin-3-yl)isoindoline-1,3-dione hydrochloride (5)

The synthetic approach to 4-(aminomethyl)-2-(2,6-dioxopiperidin-3-yl)isoindoline-1,3-dione hydrochloride can be found in Jacques, et al., Proc. Natl. Acad. Sci. 112(12), E1471-E1479 (2015), and U.S. Patent Application Publication No. 2006270707, to Zeldis et al., which are hereby incorporated by reference in their entirety. A 500 mL Parr shaker containing a solution (E)-4-((2,2-dimethylhydrazineylidene)methyl)-2-(2,6-dioxopiperidin-3-yl)isoindoline-1,3-dione, 4 (18.3 g, 55.7 mmol) in water (124 mL) and acetic acid (183 mL) was charged with palladium on carbon (750 mg, 10% by weight) under an atmosphere of nitrogen. The resulting mixture was stirred under hydrogen pressure (50 psi) for 16 hours. At that point, the catalyst was filtered through a pad of Celite and washed with methanol (100 mL). The combined filtrates were partially concentrated, then dissolved in acetonitrile (100 mL). 12M HCl (50 mL) was added and then the solution was fully concentrated under reduced pressure. The resulting residue was dissolved in methanol (100 mL) with sonication, and then cooled to 0° C. 4.5M HCl in dioxane (50 mL) was added followed by acetonitrile (20 mL) and ethyl acetate (20 mL). The resulting solution was kept in the cold room overnight, during which time a precipitate formed. This was filtered and dried under high vacuum to afford 4-(aminomethyl)-2-(2,6-dioxopiperidin-3-yl)isoindoline-1,3-dione, hydrochloride, 5 (14 g, 77.7%).

### General Procedure for HATU Mediated Amide Bond Formation

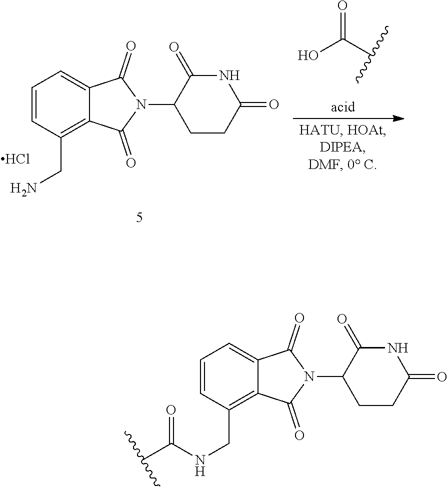

The carboxylic acid (1 eq.), 0-(7-azabenzotriazole-1-yl)-N,N,N,N′-tetramethyluronium hexafluorophosphate (HATU, 1.2 eq.) and 1-hydroxy-7-azabenzotriazole (HOAt) 0.6M in DMF (1 eq.) were dissolved in DMF. The solution was cooled to 0° C., then amine (1 equiv.) and Hunig's base (2 eq.) were added. The mixture was slowly warmed to ambient temperature and monitored for completion by LCMS (1-3 h). Once complete, the mixture was purified by preparative HPLC (column: X-Select C18 (19×150 mm, 5 μm); mobile phase A: 0.1% formic acid in water; mobile phase B: ACN; flowrate: 15 mL/min). Fractions containing the product were combined and lyophilized.

### N-((2-(2,6-dioxopiperidin-3-yl)-1,3-dioxoisoindolin-4-yl)methyl)-3,4-dihydroxybenzamide (CRB-N8046)

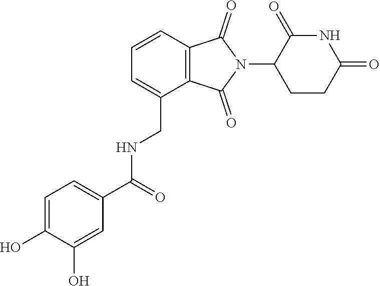 [SMILES: O=C1CCC(N2C(=O)c3cccc(CNC(=O)c4ccc(O)c(O)c4)c3C2=O)C(=O)N1]

N-((2-(2,6-dioxopiperidin-3-yl)-1,3-dioxoisoindolin-4-yl)methyl)-3,4-dihydroxybenzamide (CRB-N8046) (U.S. Patent Application Publication No. 2007049618 to Muller et al., which is hereby incorporated by reference in its entirety), was synthesized by following the general method of HATU mediated coupling of 3,4-dihydroxybenzoic acid (23.1 mg, 0.15 mmol) with 4-(aminomethyl)-2-(2,6-dioxopiperidin-3-yl)isoindoline-1,3-dione, hydrochloride, 5 (48.6 mg, 0.15 mmol). Isolated yield=22.6 mg (36%). ¹H NMR (500 MHz, DMSO-d₆) δ 11.14 (br, 1H), 8.81 (t, J=5.9 Hz, 1H), 7.86-7.77 (m, 2H), 7.68 (d, J=7.0 Hz, 1H), 7.33 (d, J=2.1 Hz, 1H), 7.27 (dd, J=8.3, 2.0 Hz, 1H), 6.78 (d, J=8.2 Hz, 1H), 5.16 (dd, J=12.8, 5.4 Hz, 1H), 4.95-4.80 (m, 2H), 2.90 (ddd, J=16.5, 13.5, 5.5 Hz, 1H), 2.67-2.52 (m, 2H), 2.12-2.03 (m, 1H). ¹³C NMR (125 MHz, DMSO-d₆) δ 172.9, 170.0, 167.7, 167.1, 166.6, 148.7, 145.0, 139.9, 134.9, 133.0, 131.6, 127.1, 125.2, 121.8, 119.2, 115.2, 115.0, 49.0, 38.4, 31.0, 22.1

### N-((2-(2,6-dioxopiperidin-3-yl)-1,3-dioxoisoindolin-4-yl)methyl)-2,3-dihydroxybenzamide (CRB-N8047)

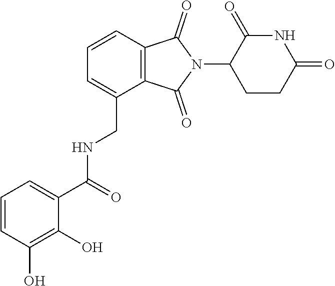 [SMILES: O=C1CCC(N2C(=O)c3cccc(CNC(=O)c4cccc(O)c4O)c3C2=O)C(=O)N1]

N-((2-(2,6-dioxopiperidin-3-yl)-1,3-dioxoisoindolin-4-yl)methyl)-2,3-dihydroxybenzamide (CRB-N8047) was synthesized by following the general procedure for mediated coupling of 2,3-dihydroxybenzoic acid (23.1 mg, 0.15 mmol) with 4-(aminomethyl)-2-(2,6-dioxopiperidin-3-yl)isoindoline-1,3-dione, hydrochloride, 5 (48.6 mg, 0.15 mmol). Isolated yield=18.3 mg (29%). ¹H NMR (500 MHz, DMSO-d₆) δ 11.16 (s, 1H), 9.46 (t, J=5.9 Hz, 1H), 7.89-7.82 (m, 2H), 7.80-7.72 (m, 1H), 7.38 (dd, J=8.1, 1.4 Hz, 1H), 6.96 (dd, J=7.8, 1.4 Hz, 1H), 6.73 (t, J=7.9 Hz, 1H), 5.19 (dd, J=12.9, 5.4 Hz, 1H), 4.98 (d, J=5.6 Hz, 2H), 2.93 (ddd, J=16.9, 13.8, 5.2 Hz, 1H), 2.69-2.55 (m, 2H), 2.14-2.04 (m, 1H). ¹³C NMR (125 MHz, DMSO-d₆) δ 172.8, 169.9, 169.9, 167.5, 167.0, 149.5, 146.3, 138.7, 134.9, 133.1, 131.6, 127.2, 122.0, 118.9, 118.1, 117.5, 115.1, 48.9, 38.2, 31.0, 22.0.

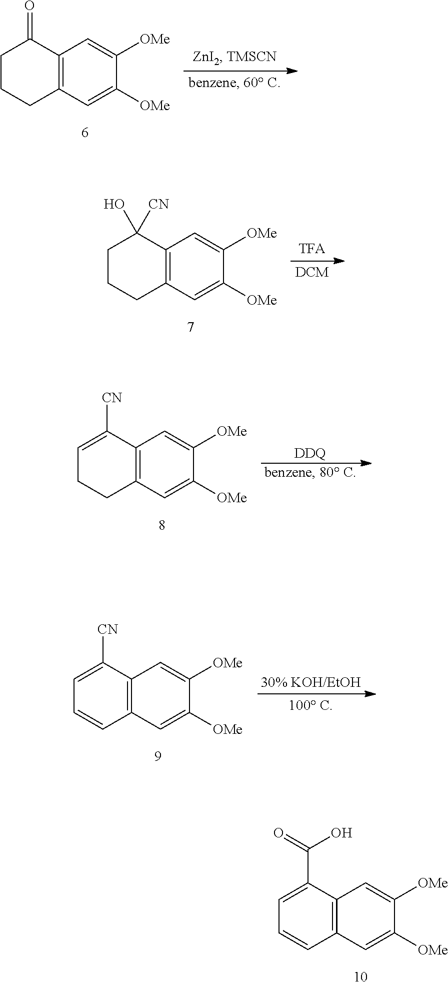

### 1-hydroxy-6,7-dimethoxy-1,2,3,4-tetrahydronaphthalene-1-carbonitrile (7)

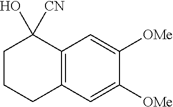 [SMILES: COc1cc2c(cc1OC)C(O)(C#N)CCC2]

To a stirred solution of 6,7-dimethoxy-3,4-dihydronaphthalen-1(2H)-one (2 g, 9.70 mmol) in dry toluene (50 mL) at ambient temperature was added zinc iodide (154 mg, 0.48 mmol) followed by trimethylsilyl cyanide (2.88 g, 29.1 mmol) under an atmosphere of nitrogen. The resulting mixture was heated at 60° C. for 16 h, at which point it was cooled to ambient temperature, diluted with water (100 mL), and extracted with ethyl acetate (3×50 mL). The combined organic layers were washed with brine (2×50 mL), dried over anhydrous sodium sulfate, filtered, and concentrated under reduced pressure. The product was purified by flash chromatography (60-120 mesh, 10% EtOAc in petroleum ether) to afford 1-hydroxy-6,7-dimethoxy-1,2,3,4-tetrahydronaphthalene-1-carbonitrile, 7 (2.3 g, 80% pure by LCMS) as a yellow oil which was subsequently used without further purification

### 6,7-dimethoxy-3,4-dihydronaphthalene-1-carbonitrile (8)

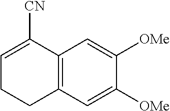 [SMILES: COc1cc2c(cc1OC)C(C#N)=CCC2]

To a stirred, 0° C. solution of 1-hydroxy-6,7-dimethoxy-1,2,3,4 tetrahydronapthalene-1-carbonitrile, 7 (2.3 g, 9.85 mmol) in dry dichloromethane (20 mL) was added trifluoroacetic acid (1.5 mL, 19 mmol) drop wise, under nitrogen atmosphere. The resulting solution was warmed to ambient temperature, stirred for 2 h, then diluted with water (50 mL), and extracted with dichloromethane (3×50 mL). The combined organic layers were washed with brine (2×50 mL), dried over anhydrous sodium sulfate, filtered, and concentrated under reduced pressure. The product was purified by flash chromatography (60-120 mesh, 10% EtOAc in petroleum ether) to afford 6,7-dimethoxy-3,4-dihydronaphthalene-1-carbonitrile, 8 (660 mg, 31%) as a white solid.

### 6,7-dimethoxy-1-naphthonitrile (9)

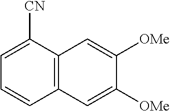 [SMILES: COc1cc2cccc(C#N)c2cc1OC]

To a stirred solution of 6,7-dimethoxy-3,4-dihydronaphthalene-1-carbonitrile, 8 (660 mg, 3.06 mmol) in dry toluene at ambient temperature was added 2,3-Dichloro-5,6-dicyano-1,4-benzoquinone (DDQ, 700 mg, 3.06 mmol) under an atmosphere of nitrogen. The resultant mixture was heated at reflux (80° C.) for 16 h, then cooled and filtered through a pad of Celite. The pad was washed with toluene (2×40 mL) and the combined filtrates were concentrated under reduced pressure. The product was purified by flash chromatography (60-120 mesh, 10% EtOAc in petroleum ether) to afford 6,7-dimethoxy-1-naphthonitrile, 9 (580 mg, 88.7%) as pale brown solid.

### 6,7-dimethoxy-1-naphthoic acid (10)

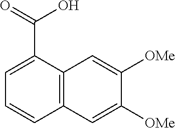 [SMILES: COc1cc2cccc(C(=O)O)c2cc1OC]

6,7-dimethoxy-1-naphthoic acid (10) (U.S. Patent Application Publication No. 2012295874, to Barany et al., which is hereby incorporated by reference in its entirety) was prepared by the following procedure. A stirred solution of 6,7-dimethoxy-1-naphthonitrile, 9 (250 mg, 1.17 mmol) in 30% KOH (3 mL) and ethanol (3 mL) was heated at 100° C. for 18 h, then cooled and concentrated under reduced pressure. The resulting residue was dissolved in water (5 mL) and extracted with dichloromethane (2×5 mL). The aqueous layer was acidified to pH 2 using conc. HCl and then extracted with ethyl acetate (2×20 mL). The combined organic layers were dried over anhydrous sodium sulfate, filtered, and concentrated under reduced pressure. The product was purified by flash chromatography (60-120 mesh, 10% EtOAc in petroleum ether) to afford 6,7-dimethoxy-1-naphthoic acid, 10 (400 mg, 64%) as an off-white solid.

### N-((2-(2,6-dioxopiperidin-3-yl)-1,3-dioxoisoindolin-4-yl)methyl)-6,7-dimethoxy-1-naphthamide (11)

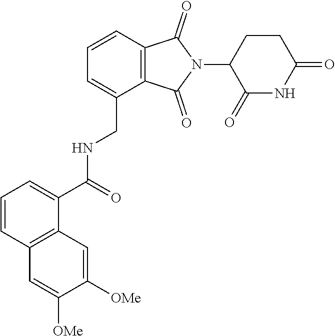 [SMILES: COc1cc2cccc(C(=O)NCc3cccc4c3C(=O)N(C3CCC(=O)NC3=O)C4=O)c2cc1OC]

N-((2-(2,6-dioxopiperidin-3-yl)-1,3-dioxoisoindolin-4-yl)methyl)-6,7-dimethoxy-1-naphthamide (11) was synthesized by following the general method of HATU mediated coupling of 6,7-dimethoxy-1-naphthoic acid, 10 (170 mg, 0.73 mmol) with 4-(aminomethyl)-2-(2,6-dioxopiperidin-3-yl)isoindoline-1,3-dione, hydrochloride, 5 (200 mg, 0.62 mmol). Isolated yield=120 mg (34%). ¹H NMR (400 MHz, DMSO-d₆): δ 8.56 (brs, 2H), 7.97-7.95 (m, 1H), 7.88-7.86 (m, 3H), 7.65 (s, 1H), 7.57 (dd, J=1.2, 7.2 Hz, 1H), 7.38 (q, J=7.2 Hz, 1H), 7.32 (s, 1H), 5.19 (q, J=5.2 Hz, 1H), 5.13 (d, J=3.6 Hz, 1H), 4.89 (s, 1H), 3.98 (s, 3H), 3.90 (s, 3H), 2.92-2.75 (m, 3H), 2.19-2.19 (m, 1H).

### General Method for BBr₃ Mediated Demethylation

To a stirred. −78° C. solution of mono- or dimethoxy intermediate (1 eq.) in dry dichloromethane (5 mL) was added BBr₃ (1M solution in DCM, 5 equiv.), under a nitrogen atmosphere. The resulting mixture was warmed to ambient temperature and stirred for 18 h. At that point, it was cooled to 0° C., quenched with saturated aqueous sodium bicarbonate (10 mL), and extracted with ethyl acetate (3×20 mL). The combined organic layers were dried over anhydrous sodium sulphate, filtered, and concentrated under reduced pressure. The product was purified by preparative HPLC [column: X-Select C18 (19×150 mm, 5 μm); mobile phase A: 0.1% formic acid in water; mobile phase B: ACN; flowrate: 15 mL/min]; fractions containing the product were combined and lyophilized.

### N-((2-(2,6-dioxopiperidin-3-yl)-1,3-dioxoisoindolin-4-yl)methyl)-6,7-dihydroxy-1-naphthamide (CRB-N8047-t27)

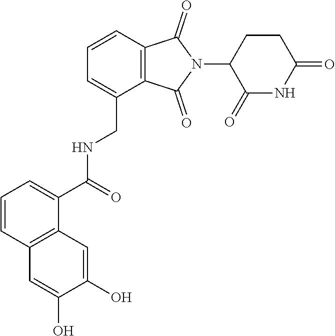 [SMILES: O=C1CCC(N2C(=O)c3cccc(CNC(=O)c4cccc5cc(O)c(O)cc45)c3C2=O)C(=O)N1]

N-((2-(2,6-dioxopiperidin-3-yl)-1,3-dioxoisoindolin-4-yl)methyl)-6,7-dihydroxy-1-naphthamide (CRB-N8047-t27) was synthesized by following the general method for BBr₃ mediated demethylation of N-((2-(2,6-dioxopiperidin-3-yl)-1,3-dioxoisoindolin-4-yl)methyl)-6,7-dimethoxy-1-naphthamide, 11 (20 mg, 0.24 mmol) with BBr₃ (1M solution in DCM, 1.19 mL, 1.19 mmol). Isolated yield=30 mg (26%). ¹H NMR (400 MHz, DMSO-d₆): δ 9.05 (brs, 1H), 8.51 (s, 1H), 7.92-7.84 (m, 3H), 7.69 (d, J=8.0 Hz, 1H), 7.59 (s, 1H), 7.45 (d, J=6.8 Hz, 1H), 7.23 (t, J=7.6 Hz, 1H), 7.15 (s, 1H), 5.19 (q, J=5.6 Hz, 1H), 4.99 (s, 2H), 3.00-2.88 (m, 1H), 2.68-2.64 (m, 2H), 2.11-2.08 (m, 1H).

### 4-chloro-N-((2-(2,6-dioxopiperidin-3-yl)-1,3-dioxoisoindolin-4-yl)methyl)-2,3-dihydroxybenzamide (CRB-N8047-t78)

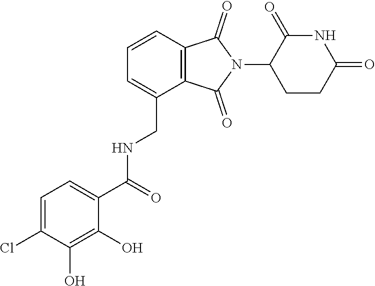 [SMILES: O=C1CCC(N2C(=O)c3cccc(CNC(=O)c4ccc(Cl)c(O)c4O)c3C2=O)C(=O)N1]

4-chloro-N-((2-(2,6-dioxopiperidin-3-yl)-1,3-dioxoisoindolin-4-yl)methyl)-2,3-dihydroxybenzamide (CRB-N8047-t78) was synthesized by following the general method of HATU mediated coupling of 4-chloro-2,3-dihydroxybenzoic acid (144 mg, 0.76 mmol) with 4-(aminomethyl)-2-(2,6-dioxopiperidin-3-yl)isoindoline-1,3-dione, hydrochloride, 5 (200 mg, 0.62 mmol). Isolated yield=18 mg (5.7%). ¹H NMR (400 MHz, DMSO-d₆): δ 13.08 (s, 1H), 11.17 (s, 1H), 9.72 (s, 1H), 9.57 (t, J=6.0 Hz, 1H), 7.85 (m, 2H), 7.75 (m, 1H), 7.43 (d, J=8.8 Hz, 1H), 6.94 (d, J=8.8 Hz, 1H), 5.21-5.16 (m, 1H), 4.98 (d, J=5.6 Hz, 2H), 2.96-2.87 (m, 1H), 2.64-2.60 (m, 2H), 2.10 (t, J=3.2 Hz, 1H).

### N-((2-(2,6-dioxopiperidin-3-yl)-1,3-dioxoisoindolin-4-yl)methyl)-2,3-dihydroxy-4-methoxybenzamide (CRB-N8047-t104)

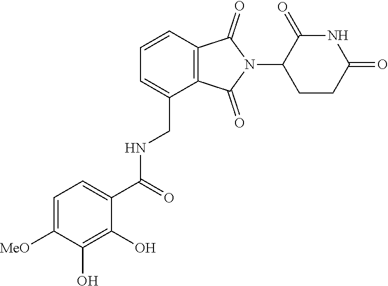 [SMILES: COc1ccc(C(=O)NCc2cccc3c2C(=O)N(C2CCC(=O)NC2=O)C3=O)c(O)c1O]

N-((2-(2,6-dioxopiperidin-3-yl)-1,3-dioxoisoindolin-4-yl)methyl)-2,3-dihydroxy-4-methoxybenzamide (CRB-N8047-t104) was synthesized by following the general method of HATU mediated coupling of 2,3-dihydroxy-4-methoxybenzoic acid (141 mg, 0.76 mmol) with 4-(aminomethyl)-2-(2,6-dioxopiperidin-3-yl)isoindoline-1,3-dione, hydrochloride, 5 (200 mg, 0.62 mmol). Isolated yield=18 mg (5.7%). ¹H NMR (400 MHz, DMSO-d₆): δ 12.40 (s, 1H), 11.16 (s, 1H), 9.29 (t, J=5.6 Hz, 1H), 8.64 (brs, 1H), 7.87-7.82 (m, 2H), 7.74-7.71 (m, 1H), 7.43 (d, J=9.2 Hz, 1H), 6.61 (d, J=9.2 Hz, 1H), 5.18 (q, J=5.6 Hz, 1H), 4.95 (d, J=5.6 Hz, 2H), 3.83 (s, 3H), 2.96-2.87 (m, 1H), 2.68-2.60 (m, 2H), 2.12-2.07 (m, 1H).

### (1R,4R)-N-((2-(2,6-dioxopiperidin-3-yl)-1,3-dioxoisoindolin-4-yl)methyl)bicyclo[2.2.1.]hept-5-ene-2-carboxamide (PKS8064)

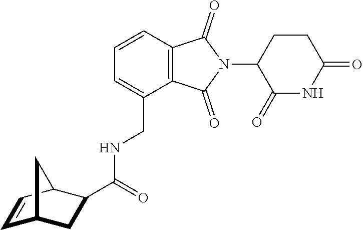

(1R,4R)-N-((2-(2,6-dioxopiperidin-3-yl)-1,3-dioxoisoindolin-4-yl)methyl)bicyclo[2.2.1]hept-5-ene-2-carboxamide (PKS8064) was synthesized by following the general method of HATU mediated coupling of 5-norbornene-2-carboxylic acid (42.7 mg, 0.31 mmol) and 4-(aminomethyl)-2-(2,6-dioxopiperidin-3-yl)isoindoline-1,3-dione, hydrochloride, 5 (100 mg, 0.31 mmol). Isolated yield=77 mg (61%). ¹H NMR (500 MHz, DMSO-d₆) δ 11.12 (s, 1H), 8.39-8.19 (m, 1H), 7.86-7.81 (m, 1H), 7.81-7.77 (m, 1H), 7.62 (d, J=7.5 Hz, 1H), 6.13 (dd, J=5.7, 3.0 Hz, 1H), 5.85 (dd, J=5.7, 2.8 Hz, 1H), 5.15 (dd, J=12.8, 5.5 Hz, 1H), 4.78-4.58 (m, 2H), 3.25-3.21 (m, 1H), 2.97-2.81 (m, 3H), 2.66-2.52 (m, 2H), 2.12-2.01 (m, 1H), 1.85-1.73 (m, 1H), 1.38-1.24 (m, 3H).

### (1S,4R,5R,6S)—N-((2-(2,6-dioxopiperidin-3-yl)-1,3-dioxoisoindolin-4-yl)methyl)-5,6-dihydroxybicyclo[2.2.1]heptane-2-carboxamide (CRB-N8066)

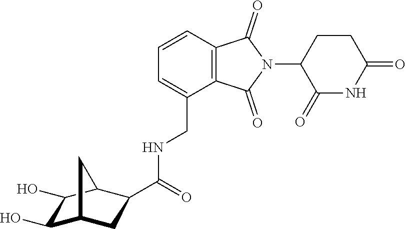 [SMILES: O=C1CCC(N2C(=O)c3cccc(CNC(=O)[C@H]4C[C@@H]5CC4[C@H](O)[C@@H]5O)c3C2=O)C(=O)N1]

To a stirred 10° C. solution of N-methylmorpholine-N-oxide (50% in water) (49.2 mg, 0.21 mmol) and osmium tetroxide (20.3 mg, 2.5 wt % in t-BuOH) in water (0.75 mL) and acetone (0.25 mL) was added (1R,4R)-N-((2-(2,6-dioxopiperidin-3-yl)-1,3-dioxoisoindolin-4-yl)methyl)bicyclo[2.2.1]hept-5-ene-2-carboxamide, PKS8064 (36.9 mg, 0.10 mmol). The resulting solution was slowly warmed to ambient temperature and stirred overnight, at which point the solvent was evaporated under reduced pressure. The product was purified by flash chromatography (60-120 mesh, 10% EtOAc in petroleum ether) to afford (1S,4R,5R,6S)—N-((2-(2,6-dioxopiperidin-3-yl)-1,3-dioxoisoindolin-4-yl)methyl)-5,6-dihydroxybicyclo[2.2.1]heptane-2-carboxamide, CRB-N8066 (34.2 mg, 49%) in a 7:3 diastereomeric ratio, as a white solid. ¹H NMR (500 MHz, DMSO-d₆) δ 11.13 (s, 1H), 8.52-8.47 (m, 0.7H), 8.45 (t, J=5.9 Hz, 0.3H), 7.88-7.77 (m, 2H), 7.68 (dd, J=7.3, 1.5 Hz, 0.7H), 7.64 (d, J=7.6 Hz, 0.3H), 5.19-5.10 (m, 1H), 4.79-4.63 (m, 2.3H), 4.62-4.56 (m, 1H), 4.52 (dd, J=5.1, 1.8 Hz, 0.7H), 3.60-3.45 (m, 2H), 2.90 (ddd, J=16.8, 13.6, 5.4 Hz, 1H), 2.70-2.52 (m, 2H), 2.31 (dd, J=4.5, 1.7 Hz, 0.7H), 2.15-2.13 (m, 0.3H), 2.10-2.01 (m, 1H), 2.01-1.95 (m, 1H), 1.80-1.68 (m, 1H), 1.58-1.41 (m, 1H), 1.26-1.10 (m, 1H).

### (1R,4R)-N-((2-(2,6-dioxopiperidin-3-yI)-1,3-dioxoisoindolin-4-yl)methyl)-7-oxobicyclo[2.2.1]hept-5-ene-2-carboxamide (CRB-N8066-t37i)

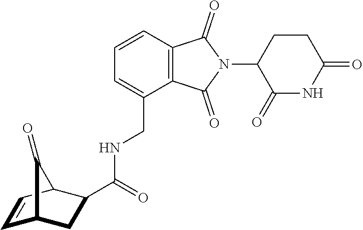

(1R,4R)-N-((2-(2,6-dioxopiperidin-3-yl)-1,3-dioxoisoindolin-4-yl)methyl)-7-oxobicyclo[2.2.1]hept-5-ene-2-carboxamide (CRB-N8066-t37i) was synthesized by following the general method of HATU mediated coupling of (1R,4R)-7-oxobicyclo[2.2.1]hept-5-ene-2-carboxylic acid (116 mg, 0.76 mmol) and 4-(aminomethyl)-2-(2,6-dioxopiperidin-3-yl)isoindoline-1,3-dione, hydrochloride, 5 (200 mg, 0.62 mmol). Isolated yield=200 mg (65% pure by LCMS).

### (1S,4R,5R,6S)—N-((2-(2,6-dioxopiperidin-3-yl)-1,3-dioxoisoindolin-4-yl)methyl)-5,6-dihydroxy-7-oxobicyclo[2.2.1]heptane-2-carboxamide (CRB-N8066-t37)

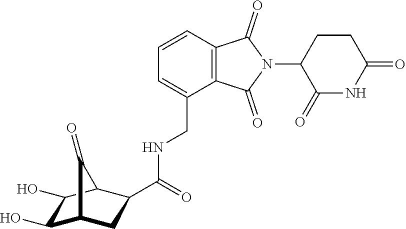 [SMILES: O=C1CCC(N2C(=O)c3cccc(CNC(=O)[C@H]4C[C@@H]5C(=O)C4[C@H](O)[C@@H]5O)c3C2=O)C(=O)N1]

To a stirred 10° C. solution of N-methylmorpholine-N-oxide (50% in water) (78 mg, 0.64 mmol) and osmium tetroxide (85 mg, 2.5 wt % in t-BuOH) in water (1 mL) and acetone (3 mL) was added (1R,4R)-N-((2-(2,6-dioxopiperidin-3-yl)-1,3-dioxoisoindolin-4-yl)methyl)-7-oxobicyclo[2.2.1]hept-5-ene-2-carboxamide, CRB-N8066-t37i (140 mg, 0.33 mmol). The resulting solution was slowly warmed to ambient temperature and stirred overnight, at which point the solvent was evaporated under reduced pressure. The product was purified by preparative HPLC [column: X-Select C18 (19×150 mm, 5 μm); mobile phase A: 0.1% formic acid in water; mobile phase B: ACN; flowrate: 15 mL/min]; fractions containing the product were combined and lyophilized to afford (1S,4R,5R,6S)—N-((2-(2,6-dioxopiperidin-3-yl)-1,3-dioxoisoindolin-4-yl)methyl)-5,6-dihydroxy-7-oxobicyclo[2.2.1]heptane-2-carboxamide, CRB-N8066-t37 (30 mg, 20%) as on off-white solid. ¹H NMR (400 MHz, DMSO-d₆): δ 8.76 (d, J=2.4 Hz, 1H), 8.50 (s, 2H), 7.84-7.69 (m, 3H), 5.17 (q, J=5.6 Hz, 2H), 4.79-4.75 (m, 2H), 3.85 (m, 1H), 2.90 (s, 2H), 2.74 (m, 2H), 2.04 (m, 1H), 1.80-1.77 (m, 1H), 1.76-1.73 (m, 1H).

### tert-butyl 2-(4-(((2-(2,6-dioxopiperidin-3-yl)-1,3-dioxoisoindolin-4-yl)methyl)carbamoyl)phenyl)hydrazine-1-carboxylate (CRB-N9101i)

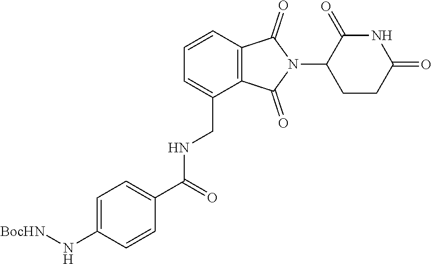 [SMILES: CC(C)(C)OC(=O)NNc1ccc(C(=O)NCc2cccc3c2C(=O)N(C2CCC(=O)NC2=O)C3=O)cc1]

tert-butyl 2-(4-(((2-(2,6-dioxopiperidin-3-yl)-1,3-dioxoisoindolin-4-yl)methyl)carbamoyl)phenyl)hydrazine-1-carboxylate (CRB-N9101i) was synthesized by following the general method of HATU mediated coupling of (4-(2-(tert-butoxycarbonyl)hydrazinyl)benzoic acid (193 mg, 0.76 mmol) and 4-(aminomethyl)-2-(2,6-dioxopiperidin-3-yl)isoindoline-1,3-dione, hydrochloride, 5 (200 mg, 0.62 mmol). Isolated yield=100 mg (95% pure by LCMS).

### N-((2-(2,6-dioxopiperidin-3-yl)-1,3-dioxoisoindolin-4-yl)methyl)-4-hydrazineylbenzamide (CRB-N9101)

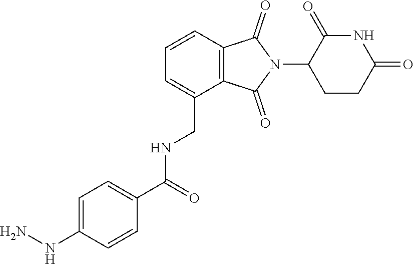 [SMILES: NNc1ccc(C(=O)NCc2cccc3c2C(=O)N(C2CCC(=O)NC2=O)C3=O)cc1]

4.5M HCl in dioxane 2 m was added to tert-butyl 2-(4-(((2-2,6-dioxopiperidin-3-yl)-1,3-dioxoisoindolin-4-yl)methyl)carbamoyl)phenyl)hydrazine-1-carboxylate, CRB-N9101i (100 mg, 0.19 mmol) at 0° C. The resulting solution was warmed to ambient temperature and stirred for 3 h, at which point it was concentrated under reduced pressure. The remaining residue was triturated with diethyl ether (10 mL) to give N-((2-(2,6-dioxopiperidin-3-yl)-1,3-dioxoisoindolin-4-yl)methyl)-4-hydrazineylbenzamide, CRB-N9101 (40 mg, 49%) as the HCl salt. ¹H NMR (400 MHz, DMSO-d₆): δ 11.15 (s, 1H), 10.11 (s, 2H), 9.00 (t, J=6.0 Hz, 1H), 8.61 (s, 1H), 7.89 (d, J=8.8 Hz, 2H), 7.84-7.81 (m, 2H), 7.73 (q, J=3.6 Hz, 1H), 6.98 (d, J=8.8 Hz, 2H), 5.18 (q, J=5.6 Hz, 1H), 4.93 (m, 2H), 2.95-2.88 (m, 1H), 2.68-2.55 (m, 2H), 2.10-2.08 (m, 1H).

### tert-butyl 2-(3-(((2-(2,6-dioxopiperidin-3-yl)-1,3-dioxoisoindolin-4-yl)methyl)carbamoyl)phenyl)hydrazine-1-carboxylate (CRB-N9102i)

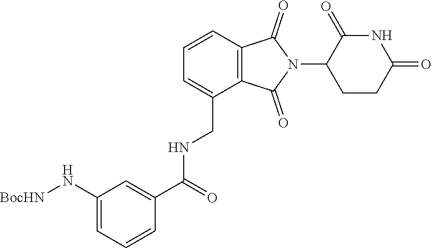 [SMILES: CC(C)(C)OC(=O)NNc1cccc(C(=O)NCc2cccc3c2C(=O)N(C2CCC(=O)NC2=O)C3=O)c1]

tert-butyl 2-(3-(((2-(2,6-dioxopiperidin-3-yl)-1,3-dioxoisoindolin-4-yl)methyl)carbamoyl)phenyl)hydrazine-1-carboxylate (CRB-N9102i) was synthesized by following the general method of HATU mediated coupling of (3-(2-(tert-butoxycarbonyl)hydrazinyl)benzoic acid (193 mg, 0.76 mmol) and 4-(aminomethyl)-2-(2,6-dioxopiperidin-3-yl)isoindoline-1,3-dione, hydrochloride, 5 (200 mg, 0.62 mmol). Isolated yield=100 mg (93% pure by LCMS).

### N-((2-(2,6-dioxopiperidin-3-yl)-1,3-dioxoisoindolin-4-yl)methyl)-3-hydrazinylbenzamide (CRB-N9102)

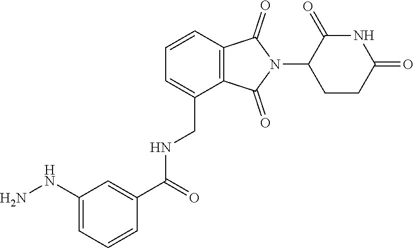 [SMILES: NNc1cccc(C(=O)NCc2cccc3c2C(=O)N(C2CCC(=O)NC2=O)C3=O)c1]

4.5M HCl in dioxane (2 ml) was added to tert-butyl 2-(3-(((2-(2,6-dioxopiperidin-3-yl)-1,3-dioxoisoindolin-4-yl)methyl)carbamoyl)phenyl)hydrazine-1-carboxylate, CRB-N9102i (100 mg, 0.19 mmol) at 0° C. The resulting solution was warmed to ambient temperature and stirred for 3 h, at which point it was concentrated under reduced pressure. The remaining residue was triturated with diethyl ether (10 mL) to give N-((2-(2,6-dioxopiperidin-3-yl)-1,3-dioxoisoindolin-4-yl)methyl)-3-hydrazinylbenzamide, CRB-N9102 (40 mg, 49%) as the HCl salt. ¹H NMR (400 MHz, DMSO-d₆): δ 11.15 (s, 1H), 10.10 (s, 3H), 9.16 (t, J=6.0 Hz, 1H), 8.40 (s, 1H), 7.84 (m, 2H), 7.72 (m, 1H), 7.56-7.52 (m, 2H), 7.44 (m, 1H), 7.13 (m, 1H), 5.18 (q, J=5.2 Hz, 1H), 4.95 (d, J=5.6 Hz, 2H), 2.93-2.89 (m, 1H), 2.67-2.58 (m, 2H), 2.11-2.08 (m, 1H).

### PKS8048

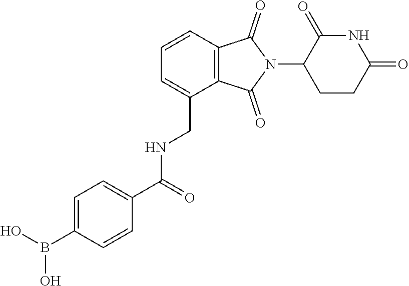 [SMILES: O=C1CCC(N2C(=O)c3cccc(CNC(=O)c4ccc(B(O)O)cc4)c3C2=O)C(=O)N1]

PKS8048 was synthesized by following the general procedure for HATU mediated coupling of 4-boronobenzoic acid (24.9 mg, 0.15 mmol) with [2-(2,6-dioxo-3-piperidyl)-1,3-dioxo-isoindolin-4-yl]methylammonium chloride (48.6 mg, 0.15 mmol). Isolated yield=52.4 mg (80%). ¹H NMR (500 MHz, DMSO-d₆) δ 11.14 (s, 1H), 9.14 (t, J=5.9 Hz, 1H), 8.21 (s, 2H), 7.91-7.85 (m, 4H), 7.86-7.80 (m, 2H), 7.74 (dd, J=6.7, 2.2 Hz, 1H), 5.18 (dd, J=12.9, 5.4 Hz, 1H), 4.99-4.89 (m, 2H), 2.91 (ddd, J=17.0, 13.8, 5.3 Hz, 1H), 2.66-2.52 (m, 2H), 2.13-2.03 (m, 1H). ¹³C NMR (125 MHz, DMSO-d₆) δ 172.8, 169.9, 167.6, 167.0, 166.8, 139.4, 137.7, 135.2, 134.8, 134.0, 133.0, 131.6, 127.1, 126.2, 121.9, 48.9, 38.35, 30.97, 22.01.

### PKS8049

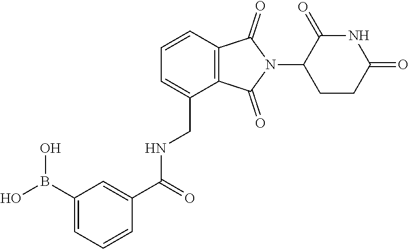 [SMILES: O=C1CCC(N2C(=O)c3cccc(CNC(=O)c4cccc(B(O)O)c4)c3C2=O)C(=O)N1]

PKS8049 was synthesized by following the general procedure for HATU mediated coupling of 3-boronobenzoic acid (24.9 mg, 0.15 mmol) with [2-(2,6-dioxo-3-piperidyl)-1,3-dioxo-isoindolin-4-yl]methylammonium chloride (48.6 mg, 0.15 mmol). Isolated yield=44.2 mg (68%). ¹H NMR (500 MHz, DMSO-d₆) δ 11.14 (s, 1H), 9.11 (t, J=5.9 Hz, 1H), 8.33 (s, 1H), 8.22 (s, 2H), 7.98-7.91 (m, 2H), 7.87-7.80 (m, 2H), 7.74 (dd, J=6.9, 1.9 Hz, 1H), 7.46 (t, J=7.5 Hz, 1H), 5.17 (dd, J=12.9, 5.4 Hz, 1H), 4.99-4.89 (m, 2H), 2.91 (ddd, J=16.8, 13.8, 5.1 Hz, 1H), 2.66-2.52 (m, 2H), 2.14-2.04 (m, 1H). ¹³C NMR (125 MHz, DMSO-d₆) δ 172.8, 169.9, 167.6, 167.2, 167.0, 139.5, 137.0, 134.8, 134.5, 133.2, 133.2, 133.0, 131.6, 128.9, 127.4, 127.1, 121.9, 48.9, 38.4, 31.0, 22.0.

### PKS8060

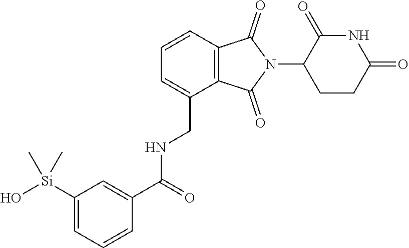 [SMILES: C[Si](C)(O)c1cccc(C(=O)NCc2cccc3c2C(=O)N(C2CCC(=O)NC2=O)C3=O)c1]

PKS8060 was synthesized by following the general procedure for HATU mediated coupling of 3-(hydroxydimethylsilyl)benzoic acid (35.0 mg, 0.178 mmol) with [2-(2,6-dioxo-3-piperidyl)-1,3-dioxo-isoindolin-4-yl]methylammonium chloride (57.7 mg, 0.178 mmol). Isolated yield=36.5 mg (44%). ¹H NMR (500 MHz, DMSO-d₆) δ 11.15 (s, 1H), 9.17 (t, J=5.9 Hz, 1H), 8.11 (s, 1H), 7.92 (dt, J=7.9, 1.6 Hz, 1H), 7.88-7.79 (m, 2H), 7.73 (dd, J=6.9, 2.8 Hz, 2H), 7.49 (t, J=7.5 Hz, 1H), 6.01 (s, 1H), 5.18 (dd, J=13.0, 5.3 Hz, 1H), 4.95 (d, J=6.1 Hz, 2H), 2.97-2.85 (m, 1H), 2.70-2.54 (m, 2H), 2.13-2.03 (m, 1H), 0.29 (s, 6H). ¹³C NMR (125 MHz, DMSO-d₆) δ 172.8, 169.9, 167.6, 167.0, 167.0, 140.9, 139.5, 136.0, 134.8, 133.0, 133.0, 131.8, 131.6, 128.0, 127.6, 127.1, 121.9, 48.9, 38.3, 31.0, 22.0, 0.6

### PKS8074

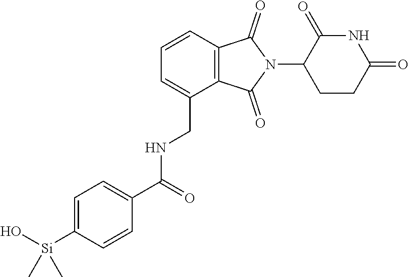 [SMILES: C[Si](C)(O)c1ccc(C(=O)NCc2cccc3c2C(=O)N(C2CCC(=O)NC2=O)C3=O)cc1]

PKS8074 was synthesized by following the general procedure for HATU mediated coupling of 4-(hydroxydimethylsilyl)benzoic acid (13.3 mg, 0.068 mmol) with [2-(2,6-dioxo-3-piperidyl)-1,3-dioxo-isoindolin-4-yl]methyl ammonium chloride (21.9 mg, 0.068 mmol). Isolated yield=25.6 mg (81%). ¹H NMR (500 MHz, DMSO-d₆) δ 11.14 (s, 1H), 9.15 (t, J=5.8 Hz, 1H), 7.90 (d, J=7.8 Hz, 2H), 7.84-7.79 (m, 2H), 7.73 (dd, J=5.9, 3.0 Hz, 1H), 7.69-7.64 (m, 2H), 6.03 (s, 1H), 5.17 (dd, J=12.9, 5.4 Hz, 1H), 5.00-4.87 (m, 2H), 2.91 (ddd, J=17.3, 14.0, 5.4 Hz, 1H), 2.66-2.54 (m, 2H), 2.12-2.04 (m, 1H), 0.35 (s, 1H), 0.27 (s, 5H). ¹³C NMR (125 MHz, DMSO-d₆) δ 173.3, 170.3, 168.0, 167.5, 167.2, 145.1, 139.9, 135.3, 134.8, 133.5, 133.4, 132.0, 127.6, 126.8, 122.4, 49.4, 38.8, 31.4, 22.5, 1.0.

### PKS8062

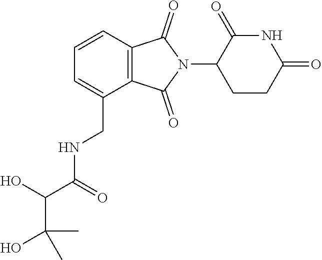 [SMILES: CC(C)(O)C(O)C(=O)NCc1cccc2c1C(=O)N(C1CCC(=O)NC1=O)C2=O]

PKS8062 was synthesized by adding 1,1I′-carbonylbis-1H-imidazole (28.54 mg, 0.176 mmol) to a solution of 2,3-dihydroxy-3-methyl-butanoic acid (21.5 mg, 0.160 mmol) in DMF (1.00 mL) at 10° C. The mixture was stirred at 10° C. for 2h and [2-(2,6-dioxo-3-piperidyl)-1,3-di oxo-isoindolin-4-yl] methyl ammonium chloride (51.8 mg, 0.160 mmol) was added. The reaction mixture was slowly allowed to warm to room temperature and stirred overnight. The mixture was purified by Autopure to give product (32.3 mg, 50%) as a white solid. ¹H NMR (500 MHz, DMSO-d₆) δ 11.13 (s, 1H), 8.53-8.44 (m, 1H), 7.85-7.77 (m, 2H), 7.73 (dd, J=7.3, 1.5 Hz, 1H), 5.71 (d, J=5.5 Hz, 1H), 5.15 (dd, J=12.8, 5.4 Hz, 1H). 4.83-4.71 (m, 2H), 4.68 (s, 1H), 3.72 (d, J=5.6 Hz, 1H), 2.90 (ddd, J=16.8, 13.6, 5.4 Hz, 1H), 2.66-2.52 (m, 2H), 2.13-2.00 (m, 1H), 1.11 (s, 3H), 1.08 (s, 3H).

### PKS8065

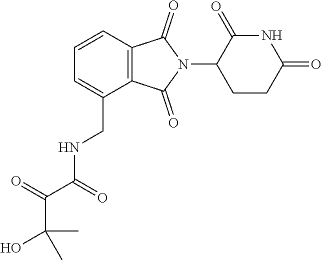 [SMILES: CC(C)(O)C(=O)C(=O)NCc1cccc2c1C(=O)N(C1CCC(=O)NC1=O)C2=O]

Dess-Martin periodinane (52.0 mg, 0.123 mmol) was added to a solution of PKS8062 (45.0 mg, 0.112 mmol) in DMSO. The reaction mixture was stirred at room temperature overnight. The mixture was purified by Autopure to give product (23.5 mg, 52%) as white solid. ¹H NMR (500 MHz, DMSO-d₆) δ 11.13 (s, 1H), 9.24 (t, J=6.1 Hz, 1H), 7.88-7.77 (m, 2H), 7.73 (dd, J=7.3, 1.4 Hz, 1H), 5.52 (s, 1H), 5.16 (dd, J=12.7, 5.4 Hz, 1H), 4.87-4.72 (m, 2H), 2.90 (ddd, J=16.8, 13.7, 5.4 Hz, 1H), 2.66-2.53 (m, 2H), 2.11-2.00 (m, 1H), 1.38 (s, 5H). ¹³C NMR (125 MHz, DMSO-d₆) δ 203.7, 172.8, 169.8, 167.5, 166.9, 165.0, 138.1, 134.8, 133.0, 131.6, 127.2, 122.1, 74.9, 48.9, 37.3, 31.0, 26.7, 22.0.

### PKS8071

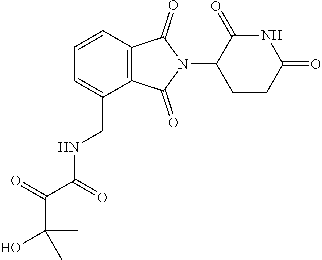 [SMILES: CC(C)(O)C(=O)C(=O)NCc1cccc2c1C(=O)N(C1CCC(=O)NC1=O)C2=O]

1,1′-Carbonylbis-1H-imidazole (77.8 mg, 0.480 mmol) was added to a solution of 2-hydroxy-2-(1-hydroxycyclobutyl)acetic acid (58.5 mg, 0.400 mmol) in DMF (2.00 mL) at 10° C. The mixture was stirred at 10° C. for 1 hour and [2-(2,6-dioxo-3-piperidyl)-1,3-dioxo-isoindolin-4-yl]methylammonium chloride(142.4 mg, 0.440 mmol) was added. The reaction mixture was slowly allowed to warm to room temperature and stirred overnight. The mixture was purified by Autopure to give product (48.2 mg, 29%) as a white solid. ¹H NMR (500 MHz, DMSO-d₆) δ 11.11 (s, 1H), 8.32 (t, J=6.4 Hz, 1H), 7.84-7.66 (m, 3H), 5.78 (d, J=5.8 Hz, 1H), 5.22 (s, 1H), 5.08 (dd, J=12.9, 5.5 Hz, 1H), 4.77-4.64 (m, 2H), 2.88-2.79 (m, 1H), 2.71-2.57 (m, 2H), 2.43-2.33 (m, 1H), 2.28-2.17 (m, 1H), 2.13-1.98 (m, 1H), 1.95-1.77 (m, 2H), 1.70-1.57 (m, 1H), 1.50-1.34 (m, 1H).

### PKS8072

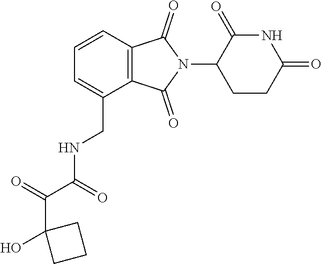 [SMILES: O=C1CCC(N2C(=O)c3cccc(CNC(=O)C(=O)C4(O)CCC4)c3C2=O)C(=O)N1]

Dess-Martin Periodinane (38.8 mg, 0.091 mmol) was added to a solution of PKS8071 (38.0 mg, 0.091 mmol) in DMSO. The reaction mixture was stirred at room temperature overnight. The mixture was purified by Autopure to give product (24.3 mg, 64%) as white solid. ¹H NMR (500 MHz, DMSO-d₆) δ 11.13 (s, 1H), 8.65 (t, J=6.3 Hz, 1H), 7.89-7.76 (m, 2H), 7.68 (d, J=7.5 Hz, 1H), 6.49 (s, 1H), 5.15 (dd, J=12.8, 5.4 Hz, 1H), 4.83-4.62 (m, 2H), 2.90 (ddd, J=16.7, 13.6, 5.4 Hz, 1H), 2.65-2.52 (m, 2H), 2.44-2.21 (m, 3H), 2.11-2.02 (m, 1H), 2.02-1.88 (m, 3H). ¹³C NMR (125 MHz, DMSO) δ 215.1, 172.8, 172.3, 169.8, 167.6, 167.0, 139.1, 134.6, 132.6, 131.5, 127.0, 121.8, 79.9, 48.9, 37.8, 36.2, 35.8, 30.9, 22.0, 18.2.

### Synthesis of VHL Targets

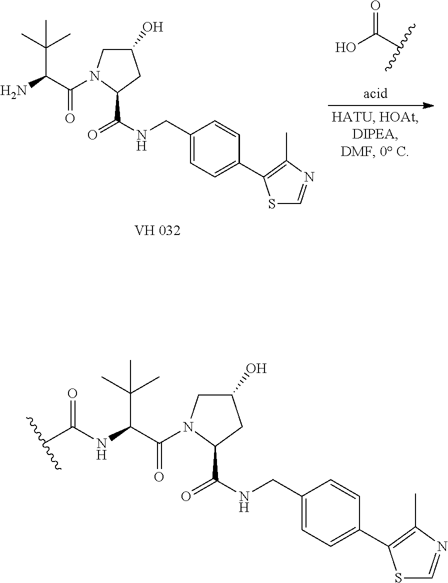

### (4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)phenyl)boronic acid (PKS8297)

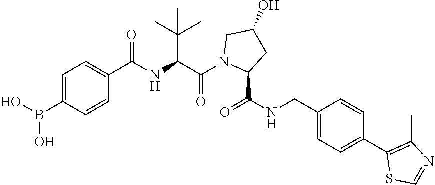 [SMILES: Cc1ncsc1-c1ccc(CNC(=O)[C@@H]2C[C@@H](O)CN2C(=O)[C@@H](NC(=O)c2ccc(B(O)O)cc2)C(C)(C)C)cc1]

(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)phenyl)boronic acid (PKS8297) was synthesized by following the general procedure for HATU mediated coupling of 4-boronobenzoic acid (3.3 mg, 20 μmol) with (2S,4R)-1-((S)-2-amino-3,3-dimethylbutanoyl)-4-hydroxy-N-(4-(4-methylthiazol-5-yl)benzyl)pyrrolidine-2-carboxamide, VH 032 (purchased from Tocris and used as received) (10 mg, 20 μmol). Isolated yield=2.6 mg (22%). ¹H NMR (500 MHz, DMSO-d₆) δ 8.98 (s, 1H), 8.58 (t, J=6.1 Hz, 1H), 8.35 (s, 2H), 7.94 (d, J=9.1 Hz, 1H), 7.85 (d, J=7.9 Hz, 2H), 7.81 (d, J=7.9 Hz, 2H), 7.45-7.37 (m, 4H), 5.15 (d, J=3.6 Hz, 1H), 4.77 (d, J=9.0 Hz, 1H), 4.51-4.32 (m, 3H), 4.24 (dd, J=15.8, 5.5 Hz, 1H), 3.73 (d, J=3.1 Hz, 2H), 2.45 (s, 3H), 2.05 (ddd, J=12.9, 7.5, 2.6 Hz, 1H), 1.92 (ddd, J=12.9, 8.6, 4.6 Hz, 1H), 1.03 (s, 9H). ¹³C NMR (125 MHz, DMSO-d₆) δ 171.9, 169.5, 166.6, 151.5, 147.7, 139.5, 137.6, 135.3, 133.9, 131.2, 129.7, 128.7, 127.5, 126.5, 68.9, 58.8, 57.3, 56.4, 41.7, 37.9, 35.6, 26.5, 15.9.

### (3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)phenyl)boronic acid (PKS8298)

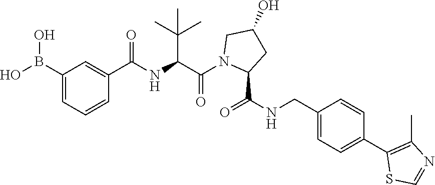 [SMILES: Cc1ncsc1-c1ccc(CNC(=O)[C@@H]2C[C@@H](O)CN2C(=O)[C@@H](NC(=O)c2cccc(B(O)O)c2)C(C)(C)C)cc1]

(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)phenyl)boronic acid (PKS8298) was synthesized by following the general procedure for HATU mediated coupling of 3-boronobenzoic acid (3.3 mg, 20 μmol) with (2S,4R)-1-((S)-2-amino-3,3-dimethylbutanoyl)-4-hydroxy-N-(4-(4-methylthiazol-5-yl)benzyl)pyrrolidine-2-carboxamide, VH 032 (10 mg, 20 μmol). Isolated yield=8.5 mg (67%). ¹H NMR (500 MHz, DMSO-d₆) δ 8.98 (s, 1H), 8.59 (t, J=6.1 Hz, 1H), 8.26 (s, 1H), 8.21 (s, 2H), 7.93-7.85 (m, 2H), 7.79 (d, J=9.2 Hz, 1H), 7.45-7.38 (m, 5H), 5.15 (d, J=3.7 Hz, 1H), 4.80 (d, J=9.2 Hz, 1H), 4.50-4.35 (m, 3H), 4.25 (dd, J=15.7, 5.6 Hz, 1H), 3.73 (d, J=3.1 Hz, 2H), 2.45 (s, 3H), 2.05 (ddd, J=13.0, 7.6, 2.6 Hz, 1H), 1.93 (ddd, J=13.0, 8.6, 4.6 Hz, 1H), 1.04 (s, 9H). ¹³C NMR (125 MHz, DMSO-d₆) δ 171.9, 169.5, 166.6, 151.5, 147.7, 139.5, 137.0, 134.2, 133.1, 132.8, 131.1, 129.7, 129.3, 128.7, 127.5, 127.4, 68.9, 58.8, 57.1, 56.5, 41.7, 37.9, 35.7, 26.5, 15.9.

### (2S,4R)-1-((S)-2-(3,4-dihydroxybenzamido)-3,3-dimethylbutanoyl)-4-hydroxy-N-(4-(4-methylthiazol-5-yl)benzyl)pyrrolidine-2-carboxamide (PKS8304)

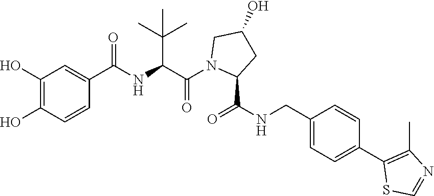 [SMILES: Cc1ncsc1-c1ccc(CNC(=O)[C@@H]2C[C@@H](O)CN2C(=O)[C@@H](NC(=O)c2ccc(O)c(O)c2)C(C)(C)C)cc1]

(2S,4R)-1-((S)-2-(3,4-dihydroxybenzamido)-3,3-dimethylbutanoyl)-4-hydroxy-N-(4-(4-methylthiazol-5-yl)benzyl)pyrrolidine-2-carboxamide (PKS8304) was synthesized by following the general procedure for HATU mediated coupling of 3,4-dihydroxybenzoic acid (15.4 mg, 100 μmol) with (2S,4R)-1-((S)-2-amino-3,3-dimethylbutanoyl)-4-hydroxy-N-(4-(4-methylthiazol-5-yl)benzyl)pyrrolidine-2-carboxamide, VH 032 (10 mg, 20 μmol). Isolated yield=4.4 mg (39%). ¹H NMR (500 MHz, DMSO-d₆) δ 8.92 (s, 1H), 8.51 (t, J=6.2 Hz, 1H), 7.40-7.28 (m, 5H), 7.20 (d, J=2.2 Hz, 1H), 7.15 (dd, J=8.3, 2.1 Hz, 1H), 6.69 (d, J=8.2 Hz, 1H), 5.08 (s, 1H), 4.64 (d, J=9.1 Hz, 1H), 4.41-4.27 (m, 3H), 4.18 (dd, J=15.8, 5.6 Hz, 1H), 3.64 (d, J=2.9 Hz, 2H), 2.38 (s, 3H), 2.02-1.93 (m, 1H), 1.84 (ddd, J=13.0, 8.6, 4.6 Hz, 1H), 0.94 (s, 9H). ¹³C NMR (125 MHz, DMSO-d₆) δ 171.9, 169.7, 166.0, 151.5, 148.6, 147.7, 144.9, 139.5, 131.2, 129.7, 128.7, 127.5, 125.1, 119.2, 115.1, 114.9, 68.9, 58.8, 56.9, 56.4, 41.7, 37.9, 35.6, 26.5, 15.9.

### (2S,4R)-1-((S)-2-(2,3-dihydroxybenzamido)-3,3-dimethylbutanoyl)-4-hydroxy-N-(4-(4-methylthiazol-5-yl)benzyl)pyrrolidine-2-carboxamide (PKS8305)

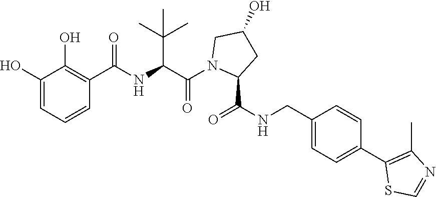 [SMILES: Cc1ncsc1-c1ccc(CNC(=O)[C@@H]2C[C@@H](O)CN2C(=O)[C@@H](NC(=O)c2cccc(O)c2O)C(C)(C)C)cc1]

(2S,4R)-1-((S)-2-(2,3-dihydroxybenzamido)-3,3-dimethylbutanoyl)-4-hydroxy-N-(4-(4-methylthiazol-5-yl)benzyl)pyrrolidine-2-carboxamide (PKS8305) was synthesized by following the general procedure for HATU mediated coupling of 2,3-dihydroxybenzoic acid (15.4 mg, 100 μmol) with (2S,4R)-1-((S)-2-amino-3,3-dimethylbutanoyl)-4-hydroxy-N-(4-(4-methylthiazol-5-yl)benzyl)pyrrolidine-2-carboxamide, VH 032 (10 mg, 20 μmol). Isolated yield =3.5 mg (39%). ¹H NMR (500 MHz, DMSO-d₆) δ 10.71 (s, 1H), 9.65 (s, 1H), 8.99 (s, 1H), 8.74 (d, J=9.1 Hz, 1H), 8.58 (t, J=6.2 Hz, 1H), 7.44-7.37 (m, 4H), 7.35-7.28 (m, 1H), 6.93 (dd, J=7.8, 1.6 Hz, 1H), 6.71 (t, J=7.9 Hz, 1H), 5.15 (d, J=3.6 Hz, 1H), 4.78 (d, J=9.1 Hz, 1H), 4.45 (t, J=8.0 Hz, 1H), 4.42-4.34 (m, 2H), 4.27 (dd, J=15.8, 5.7 Hz, 1H), 3.72 (d, J=3.0 Hz, 2H), 2.45 (s, 3H), 2.05 (ddd, J=12.8, 7.6, 2.5 Hz, 1H), 1.92 (ddd, J=12.8, 8.6, 4.5 Hz, 1H), 1.01 (s, 9H). ¹³C NMR (125 MHz, DMSO-d₆) δ 171.8, 169.4, 165.7, 151.5, 147.8, 146.1, 145.8, 139.5, 131.2, 129.7, 128.7, 127.5, 120.0, 118.7, 118.2, 118.1, 68.9, 58.8, 56.8, 41.7, 40.1, 37.9, 35.6, 26.4, 16.0.

### Synthesis of MDM2 Targets

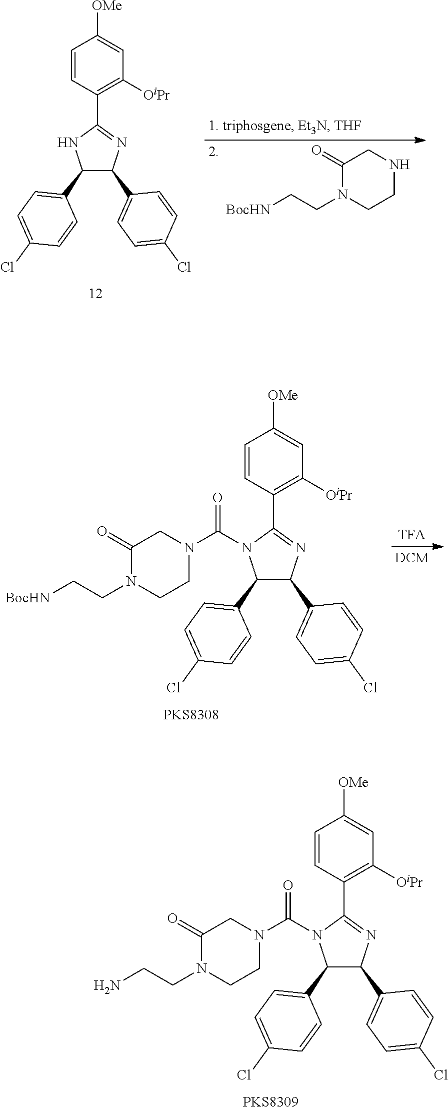 [SMILES: COc1ccc(C2=N[C@@H](c3ccc(Cl)cc3)[C@@H](c3ccc(Cl)cc3)N2)c(OC(C)C)c1 ; CC(C)(C)OC(=O)NCCN1CCNCC1=O ; COc1ccc(C2=N[C@@H](c3ccc(Cl)cc3)[C@@H](c3ccc(Cl)cc3)N2C(=O)N2CCN(CCNC(=O)OC(C)(C)C)C(=O)C2)c(OC(C)C)c1 ; COc1ccc(C2=N[C@@H](c3ccc(Cl)cc3)[C@@H](c3ccc(Cl)cc3)N2C(=O)N2CCN(CCN)C(=O)C2)c(OC(C)C)c1]

### tert-butyl (2-(4-((4S,5R)-4,5-bis(4-chlorophenyl)-2-(2-isopropoxy-4-methoxyphenyl)-4,5-dihydro-1H-imidazole-1-carbonyl)-2-oxopiperazin-1-yl)ethyl)carbamate (PKS8308)

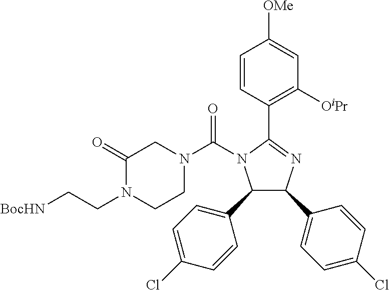 [SMILES: COc1ccc(C2=N[C@@H](c3ccc(Cl)cc3)[C@@H](c3ccc(Cl)cc3)N2C(=O)N2CCN(CCNC(=O)OC(C)(C)C)C(=O)C2)c(OC(C)C)c1]

((4S,5R)-4,5-bis(4-chlorophenyl)-2-(2-isopropoxy-4-methoxyphenyl)-4,5-dihydro-1H-imidazole, 12 (45 mg, 100 μmol) was dissolved in THF (1 mL) and cooled to 0° C. Triethylamine (50 mg, 0.50 mmol) and triphosgene (147 mg, 0.50 mmol) were added to the solution. The mixture was stirred at 0° C. for 3 h and the solvent was removed under reduced pressure. To the residue dissolved in DCM (2 mL) at 0° C. was added dropwise a solution of tert-butyl N-[2-(2-oxopiperazin-1-yl)ethyl]carbamate (WO2017025868, to Ninkovic et al., which is hereby incorporated by reference in its entirety) (240 mg, 0.10 mmol) in THE (1 mL). The resulting mixture was stirred at 0° C. for 2 h. The mixture was quenched with saturated sodium bicarbonate solution (25 mL) and extracted with dichloromethane (2×25 mL). The combined organic layers were washed with brine, dried over Na₂SO₄, filtered, and concentrated under reduced pressure. The residue was purified by flash chromatography (0-10% MeOH in DCM) to give tert-butyl (2-(4-((4S,5R)-4,5-bis(4-chlorophenyl)-2-(2-isopropoxy-4-methoxyphenyl)-4,5-dihydro-1H-imidazole-1-carbonyl)-2-oxopiperazin-1-yl)ethyl)carbamate, PKS8308 (67 mg, 94%) as a white solid. ¹H NMR (500 MHz, DMSO-d₆) δ 7.53 (d, J=8.3 Hz, 1H), 7.15 (d, J=8.1 Hz, 2H), 7.11 (d, J=8.1 Hz, 2H), 7.04 (d, J=8.1 Hz, 2H), 6.97 (d, J=8.1 Hz, 2H), 6.78 (t, J=6.0 Hz, 1H), 6.65-6.57 (m, 2H), 5.65 (d, J=9.7 Hz, 1H), 5.58 (d, J=9.7 Hz, 1H), 4.77-4.66 (m, 1H), 3.82 (s, 3H), 3.68 (d, J=17.4 Hz, 1H), 3.52 (d, J=17.4 Hz, 1H), 3.32 (s, 1H), 3.25-3.07 (m, 3H), 3.01-2.87 (m, 4H), 1.33 (s, 9H), 1.26 (d, J=6.0 Hz, 3H), 1.21 (d, J=5.9 Hz, 3H)

### 1-(2-aminoethyl)-4-((4S,5R)-4,5-bis(4-chlorophenyl)-2-(2-isopropoxy-4-methoxyphenyl)-4,5-dihydro-1H-imidazole-1-carbonyl)piperazin-2-one (PKS8309)

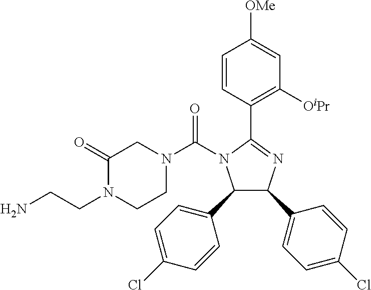 [SMILES: COc1ccc(C2=N[C@@H](c3ccc(Cl)cc3)[C@@H](c3ccc(Cl)cc3)N2C(=O)N2CCN(CCN)C(=O)C2)c(OC(C)C)c1]

tert-butyl (2-(4-((4S,5R)-4,5-bis(4-chlorophenyl)-2-(2-isopropoxy-4-methoxyphenyl)-4,5-dihydro-1H-imidazole-1-carbonyl)-2-oxopiperazin-1-yl)ethyl)carbamate, PKS8308 (67 mg, 92 μmol) was dissolved in DCM (2 mL) and the solution was cooled to 0° C. Trifluoroacetic acid (0.5 mL) was added dropwise with constant stirring. The resultant solution was warmed slowly to ambient temperature. Upon reaction completion, excess trifluoroacetic acid and dichloromethane were evaporated under reduced pressure and the remaining residue triturated with diethyl ether to give a white solid. The solid was filtered and then dried under vacuum to give 1-(2-aminoethyl)-4-((4S,5R)-4,5-bis(4-chlorophenyl)-2-(2-isopropoxy-4-methoxyphenyl)-4,5-dihydro-1H-imidazole-1-carbonyl)piperazin-2-one, PKS8309 (65.2 mg, 95%) as an off-white solid, which was subsequently used without further purification. ¹H NMR (500 MHz, DMSO-d₆) δ 7.70 (t, J=6.0 Hz, 3H), 7.65 (d, J=8.6 Hz, 1H), 7.24 (d, J=8.1 Hz, 2H), 7.19 (d, J=8.1 Hz, 2H), 7.10 (d, J=8.1 Hz, 2H), 7.02 (d, J=8.1 Hz, 2H), 6.79-6.69 (m, 2H), 6.04-5.93 (m, 1H), 5.93-5.83 (m, 1H), 4.89-4.80 (m, 1H), 3.87 (s, 3H), 3.85-3.80 (m, 1H), 3.62 (d, J=17.4 Hz, 1H), 3.43 (s, 2H), 3.37-3.29 (m, 1H), 3.29-3.20 (m, 1H), 3.12-2.99 (m, 2H), 2.95-2.85 (m, 2H), 1.32 (d, J=6.0 Hz, 3H), 1.27 (d, J=6.0 Hz, 3H).

### (4-((2-(4-((4S,5R)-4,5-bis(4-chlorophenyl)-2-(2-isopropoxy-4-methoxyphenyl)-4,5-dihydro-1H-imidazole-1-carbonyl)-2-oxopiperazin-1-yl)ethyl)carbamoyl)phenyl)boronic acid (PKS8310)

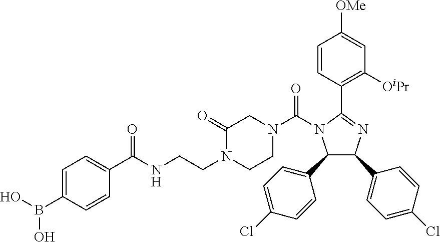 [SMILES: COc1ccc(C2=N[C@@H](c3ccc(Cl)cc3)[C@@H](c3ccc(Cl)cc3)N2C(=O)N2CCN(CCNC(=O)c3ccc(B(O)O)cc3)C(=O)C2)c(OC(C)C)c1]

(4-((2-(4-((4S,5R)-4,5-bis(4-chlorophenyl)-2-(2-isopropoxy-4-methoxyphenyl)-4,5-dihydro-1H-imidazole-1-carbonyl)-2-oxopiperazin-1-yl)ethyl)carbamoyl)phenyl)boronic acid (PKS8310) was synthesized by following the general procedure for HATU mediated coupling of 4-boronobenzoic acid (6.6 mg, 40 μmol) with 1-(2-aminoethyl)-4-((4S,5R)-4,5-bis(4-chlorophenyl)-2-(2-isopropoxy-4-methoxyphenyl)-4,5-dihydro-1H-imidazole-1-carbonyl)piperazin-2-one, PKS8309 (15 mg, 20 μmol). Isolated yield=6 mg (39%). ¹H NMR (500 MHz, DMSO-d₆) δ 8.47 (t, J=5.8 Hz, 1H), 8.21 (s, 2H), 7.84 (d, J=7.7 Hz, 2H), 7.72 (d, J=7.9 Hz, 2H), 7.52 (d, J=8.3 Hz, 1H), 7.15 (d, J=8.0 Hz, 2H), 7.10 (d, J=8.1 Hz, 2H), 7.03 (d, J=8.1 Hz, 2H), 6.96 (d, J=8.1 Hz, 2H), 6.65-6.58 (m, 2H), 5.63 (d, J=9.7 Hz, 1H), 5.56 (d, J=9.7 Hz, 1H), 4.70 (hept, J=6.1 Hz, 1H), 3.82 (s, 3H), 3.69 (d, J=17.4 Hz, 1H), 3.53 (d, J=17.4 Hz, 1H), 3.43-3.25 (m, 5H), 3.20-3.11 (m, 1H), 3.03 (t, J=5.5 Hz, 2H), 1.25 (d, J=5.9 Hz, 3H), 1.20 (d, J=5.9 Hz, 3H). ¹³C NMR (125 MHz, DMSO-d₆) δ 166.5, 164.2, 162.3, 160.0, 156.5, 154.3, 137.4, 136.4, 135.6, 133.9, 131.9, 131.3, 131.1, 129.7, 128.7, 127.4, 127.4, 125.9, 113.4, 104.9, 99.3, 71.2, 69.8, 67.8, 55.4, 49.0, 45.8, 45.6, 42.2, 36.6, 21.7, 21.6

### (3-((2-(4-((4S,5R)-4,5-bis(4-chlorophenyl)-2-(2-isopropoxy-4-methoxyphenyl)-4,5-dihydro-1H-imidazole-1-carbonyl)-2-oxopiperazin-1-yl)ethyl)carbamoyl)phenyl)boronic acid (PKS8312)

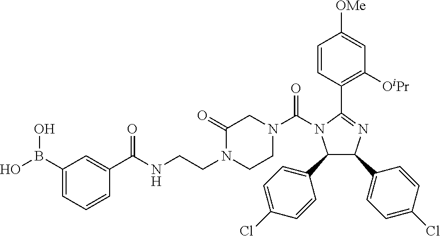 [SMILES: COc1ccc(C2=N[C@@H](c3ccc(Cl)cc3)[C@@H](c3ccc(Cl)cc3)N2C(=O)N2CCN(CCNC(=O)c3cccc(B(O)O)c3)C(=O)C2)c(OC(C)C)c1]

(3-((2-(4-((4S,5R)-4,5-bis(4-chlorophenyl)-2-(2-isopropoxy-4-methoxyphenyl)-4,5-dihydro-1H-imidazole-1-carbonyl)-2-oxopiperazin-1-yl)ethyl)carbamoyl)phenyl)boronic acid (PKS8312) was synthesized by following the general procedure for HATU mediated coupling of 3-boronobenzoic acid (6.6 mg, 40 μmol) with 1-(2-aminoethyl)-4-((4S,5R)-4,5-bis(4-chlorophenyl)-2-(2-isopropoxy-4-methoxyphenyl)-4,5-dihydro-1H-imidazole-1-carbonyl)piperazin-2-one, PKS8309 (15 mg, 20 μmol). Isolated yield=10 mg (65%). ¹H NMR (500 MHz, DMSO-d₆) δ 8.46 (t, J=5.5 Hz, 1H), 8.31 (s, 2H), 8.21 (s, 1H), 7.91 (d, J=7.3 Hz, 1H), 7.77 (d, J=7.8 Hz, 1H), 7.52 (d, J=8.4 Hz, 1H), 7.41 (t, J=7.6 Hz, 1H), 7.15 (d, J=8.2 Hz, 2H), 7.09 (d, J=8.1 Hz, 2H), 7.03 (d, J=8.1 Hz, 2H), 6.95 (d, J=8.1 Hz, 2H), 6.66-6.55 (m, 2H), 5.62 (d, J=9.7 Hz, 1H), 5.56 (d, J=9.7 Hz, 1H), 4.76-4.65 (m, 1H), 3.81 (s, 3H), 3.69 (d, J=17.4 Hz, 1H), 3.53 (d, J=17.4 Hz, 1H), 3.45-3.23 (m, 5H), 3.20-3.11 (m, 1H), 3.02 (t, J=5.4 Hz, 2H), 1.24 (d, J=6.0 Hz, 3H), 1.19 (d, J=5.9 Hz, 3H). ¹³C NMR (125 MHz, DMSO-d₆) δ 166.9, 164.1, 162.3, 160.0, 156.5, 154.3, 137.4, 136.7, 136.4, 133.6, 133.0, 132.0, 131.3, 131.1, 129.7, 128.7, 128.6, 127.4, 127.4, 127.3, 113.4, 104.9, 99.3, 71.2, 69.8, 67.8, 55.4, 49.0, 45.8, 45.7, 42.2, 36.6, 21.7, 21.6.

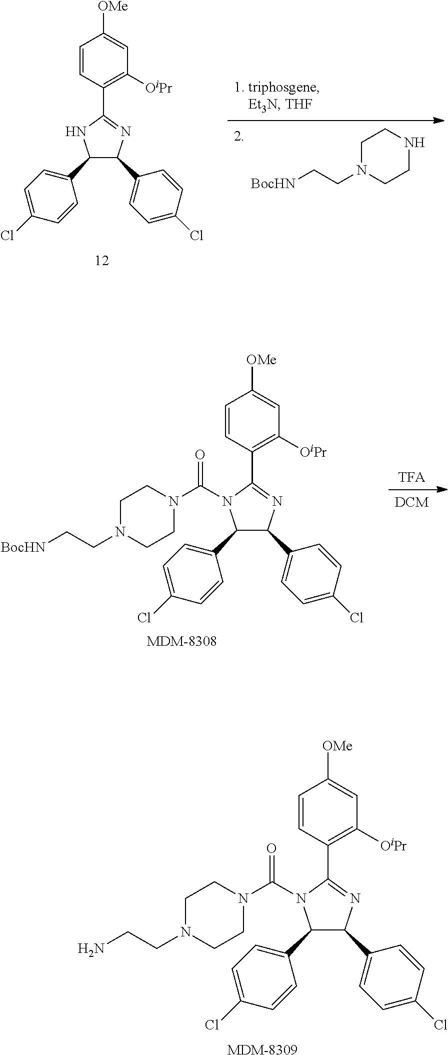

### tert-butyl (2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)carbamate (MDM-8308)

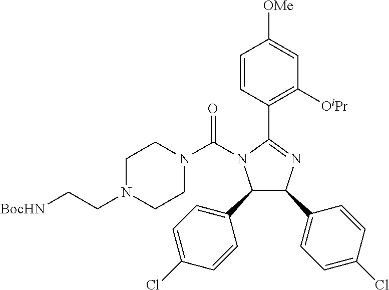 [SMILES: COc1ccc(C2=N[C@@H](c3ccc(Cl)cc3)[C@@H](c3ccc(Cl)cc3)N2C(=O)N2CCN(CCNC(=O)OC(C)(C)C)CC2)c(OC(C)C)c1]

((4S,5R)-4,5-bis(4-chlorophenyl)-2-(2-isopropoxy-4-methoxyphenyl)-4,5-dihydro-1H-imidazole, 12 (200 mg, 440 μmol) was dissolved in THE (10 mL) and cooled to 0° C. Triethylamine (220 mg, 2.19 mmol) and triphosgene (390 mg, 1.31 mmol) were added to the solution. The mixture was stirred at 0° C. for 3 h and the solvent was removed under reduced pressure. To the residue dissolved in DCM (20 mL) at 0° C. was added dropwise a solution of tert-butyl (2-(piperazin-1-yl)ethyl)carbamate (503 mg, 2.19 mmol) in DCM (10 mL). The resulting mixture was stirred at 0° C. for 2 h. The mixture was quenched with saturated sodium bicarbonate solution (25 mL) and extracted with dichloromethane (2×25 mL). The combined organic layers were washed with brine, dried over Na₂SO₄, filtered, and concentrated under reduced pressure. The residue was purified by flash chromatography (0-10% MeOH in DCM) to give tert-butyl (2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)carbamate, MDM-8308 (300 mg, 96%) as a white solid.

### (4-(2-aminoethyl)piperazin-1-yl)((4S,5R)-4,5-bis(4-chlorophenyl)-2-(2-isopropoxy-4-methoxyphenyl)-4,5-dihydro-1H-imidazol-1-yl)methanone (MDM-8309)

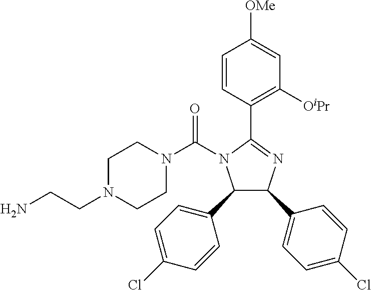 [SMILES: COc1ccc(C2=N[C@@H](c3ccc(Cl)cc3)[C@@H](c3ccc(Cl)cc3)N2C(=O)N2CCN(CCN)CC2)c(OC(C)C)c1]

tert-butyl (2-(4-((4S,5R)-4,5-bis 4-chlorophenyl-2-2-isopropoxy-4-methoxyphenyl)-4,5-dihydro-1H-imidazole-1-carbonyl)piperazin-1-yl)ethyl)carbamate, MDM-8308 (300 mg, 422 μmol) was dissolved in DCM (5 mL) and the solution was cooled to 0° C. Trifluoroacetic acid (0.5 mL) was added dropwise with constant stirring. The resultant solution was warmed slowly to ambient temperature. Upon reaction completion, excess trifluoroacetic acid and dichloromethane were evaporated under reduced pressure and the remaining residue triturated with diethyl ether to give a white solid. The solid was filtered and then dried under vacuum to give (4-(2-aminoethyl)piperazin-1-yl)((4S,5R)-4,5-bis(4-chlorophenyl)-2-(2-isopropoxy-4-methoxyphenyl)-4,5-dihydro-1H-imidazol-1-yl)methanone, MDM-8309 (240 mg, 93%) as an off-white solid, which was subsequently used without further purification.

### (4-((2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)carbamoyl)phenyl)boronic acid (MDM-8310)

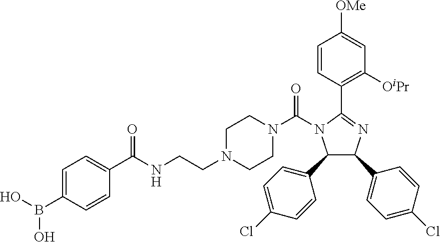 [SMILES: COc1ccc(C2=N[C@@H](c3ccc(Cl)cc3)[C@@H](c3ccc(Cl)cc3)N2C(=O)N2CCN(CCNC(=O)c3ccc(B(O)O)cc3)CC2)c(OC(C)C)c1]

(4-((2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)carbamoyl)phenyl)boronic acid (MDM-8310) was synthesized by following the general method of HATU mediated coupling of 4-boronobenzoic acid (29 mg, 0.17 mmol) with 4-(2-aminoethyl)piperazin-1-yl)((4S,5R)-4,5-bis(4-chlorophenyl)-2-(2-isopropoxy-4-methoxyphenyl)-4,5-dihydro-1H-imidazol-1-yl)methanone, MDM-8309 (100 mg, 0.16 mmol). Isolated yield=50 mg (40%). ¹H NMR (400 MHz, DMSO-d₆) δ 10.55 (brs, 1H), 8.75 (brs, 1H), 8.27 (brs, 2H), 7.97-7.83 (m, 3H), 7.67 (m, 1H), 7.27-7.20 (m, 4H), 7.15-7.02 (m, 4H), 6.78 (m, 2H), 5.98 (m, 2H), 4.88 (t, J=5.2 Hz, 1H), 3.84 (m, 3H), 3.77 (m, 4H), 3.66 (m, 2H), 3.60 (m, 2H), 3.18 (m, 2H), 2.94 (m, 2H), 1.35 (d, J=6.0 Hz, 3H), 1.27 (d, J=5.6 Hz, 3H)

### (3-((2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)carbamoyl)phenyl)boronic acid (MDM-8312)

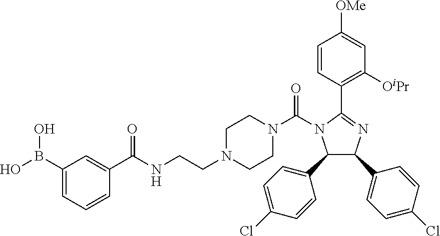 [SMILES: COc1ccc(C2=N[C@@H](c3ccc(Cl)cc3)[C@@H](c3ccc(Cl)cc3)N2C(=O)N2CCN(CCNC(=O)c3cccc(B(O)O)c3)CC2)c(OC(C)C)c1]

(3-((2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)carbamoyl)phenyl)boronic acid (MDM-8312) was synthesized by following the following the general method of HATU mediated coupling of 3-boronobenzoic acid (29 mg, 0.17 mmol) with 4-(2-aminoethyl)piperazin-1-yl)((4S,5R)-4,5-bis(4-chlorophenyl)-2-(2-isopropoxy-4-methoxyphenyl)-4,5-dihydro-1H-imidazol-1-yl)methanone, MDM-8309 (100 mg, 0.16 mmol). Isolated yield=25 mg (20%). ¹H NMR (400 MHz, DMSO-d₆) δ 8.35 (m, 1H), 8.23 (brs, 1H), 8.15 (s, 2H), 7.84 (d, J=8.4 Hz, 2H), 7.75 (d, J=8.0 Hz, 2H), 7.46 (d, J=8.4 Hz, 1H), 7.14 (dd, J=8.4, 16.8 Hz, 4H), 7.04 (d, J=8.4 Hz, 2H), 6.96 (d, J=8.0 Hz, 2H), 6.65-6.61 (m, 2H), 5.64 (d, J=10.0 Hz, 1H), 5.52 (d, J=10.0 Hz, 1H), 4.73-4.70 (m, 1H), 3.82 (s, 3H), 3.03 (m, 4H), 2.68 (m, 1H), 2.34-2.28 (m, 3H), 2.01 (m, 4H), 1.28 (d, J=6.0 Hz, 3H), 1.24 (d, J=6.0 Hz, 3H).

### N-(2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)-3,4-dihydroxybenzamide (MDM-8313)

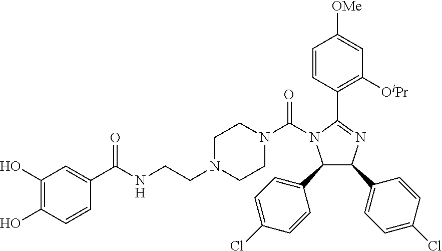 [SMILES: COc1ccc(C2=N[C@@H](c3ccc(Cl)cc3)[C@@H](c3ccc(Cl)cc3)N2C(=O)N2CCN(CCNC(=O)c3ccc(O)c(O)c3)CC2)c(OC(C)C)c1]

N-(2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)-3,4-dihydroxybenzamide (MDM-8313) was synthesized by following the general method of HATU mediated coupling of 3,4-dihydroxy benzoic acid (27 mg, 0.17 mmol) with 4-(2-aminoethyl)piperazin-1-yl)((4S,5R)-4,5-bis(4-chlorophenyl)-2-(2-isopropoxy-4-methoxyphenyl)-4,5-dihydro-1H-imidazol-1-yl)methanone, MDM-8309 (100 mg, 0.16 mmol). Isolated yield=20 mg (16%). ¹H NMR (400 MHz, DMSO-d₆) δ 9.15 (brs, 1H), 8.27 (s, 1H), 8.00 (t, J=5.6 Hz, 1H), 7.46 (d, J=8.4 Hz, 1H), 7.23 (d, J=2.0 Hz, 1H), 7.17-7.11 (m, 5H), 7.04 (m, 2H), 6.96 (m, 2H), 6.74 (m, 1H), 6.65-6.61 (m, 1H), 5.64 (d, J=10.0 Hz, 1H), 5.52 (d, J=10.0 Hz, 1H), 4.71 (m, 1H), 3.83 (s, 3H), 3.34-3.17 (m, 2H), 3.03 (m, 4H), 2.34 (m, 2H), 2.00 (m, 4H), 1.28 (d, J=6.0 Hz, 3H), 1.24 (d, J=6.0, 3H).

### N-(2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)-2,3-dihydroxybenzamide (MDM-8314)

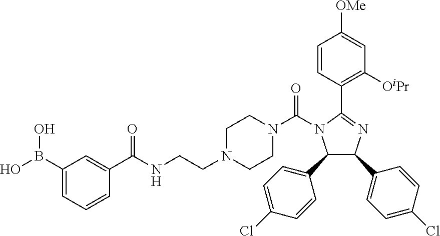 [SMILES: COc1ccc(C2=N[C@@H](c3ccc(Cl)cc3)[C@@H](c3ccc(Cl)cc3)N2C(=O)N2CCN(CCNC(=O)c3cccc(B(O)O)c3)CC2)c(OC(C)C)c1]

N-(2-(4-((4S,5R)-4,5-bis(4-chlorophenyl)-2-(2-isopropoxy-4-methoxyphenyl)-4,5-dihydro-1H-imidazole-1-carbonyl)piperazin-1-yl)ethyl)-2,3-dihydroxybenzamide (MDM-8314) was synthesized by following the general method of HATU mediated coupling of 2,3-dihydroxy benzoic acid (40 mg, 0.25 mmol) with 4-(2-aminoethyl)piperazin-1-yl)((4S,5R)-4,5-bis(4-chlorophenyl)-2-(2-isopropoxy-4-methoxyphenyl)-4,5-dihydro-1H-imidazol-1-yl)methanone, MDM-8309 (150 mg, 0.24 mmol). Isolated yield=10 mg (5.4%). ¹H NMR (400 MHz, DMSO-d₆) δ 9.04 (s, 1H), 8.35 (s, 1H), 7.46 (m, 1H), 7.20-7.11 (m, 6H), 7.04 (m, 2H), 6.96 (m, 2H), 6.85 (m, 1H), 6.65-6.58 (m, 3H), 5.64 (d, J=10.0 Hz, 1H), 5.52 (d, J=10.0 Hz, 1H), 4.72 (m, 1H), 3.83 (s, 3H), 3.04 (m, 4H), 2.34-2.28 (m, 2H), 2.32 (m, 4H), 1.28 (d, J=6.0 Hz, 3H), 1.24 (d, J=6.0 Hz, 3H).

### Synthesis of BRD-E50c

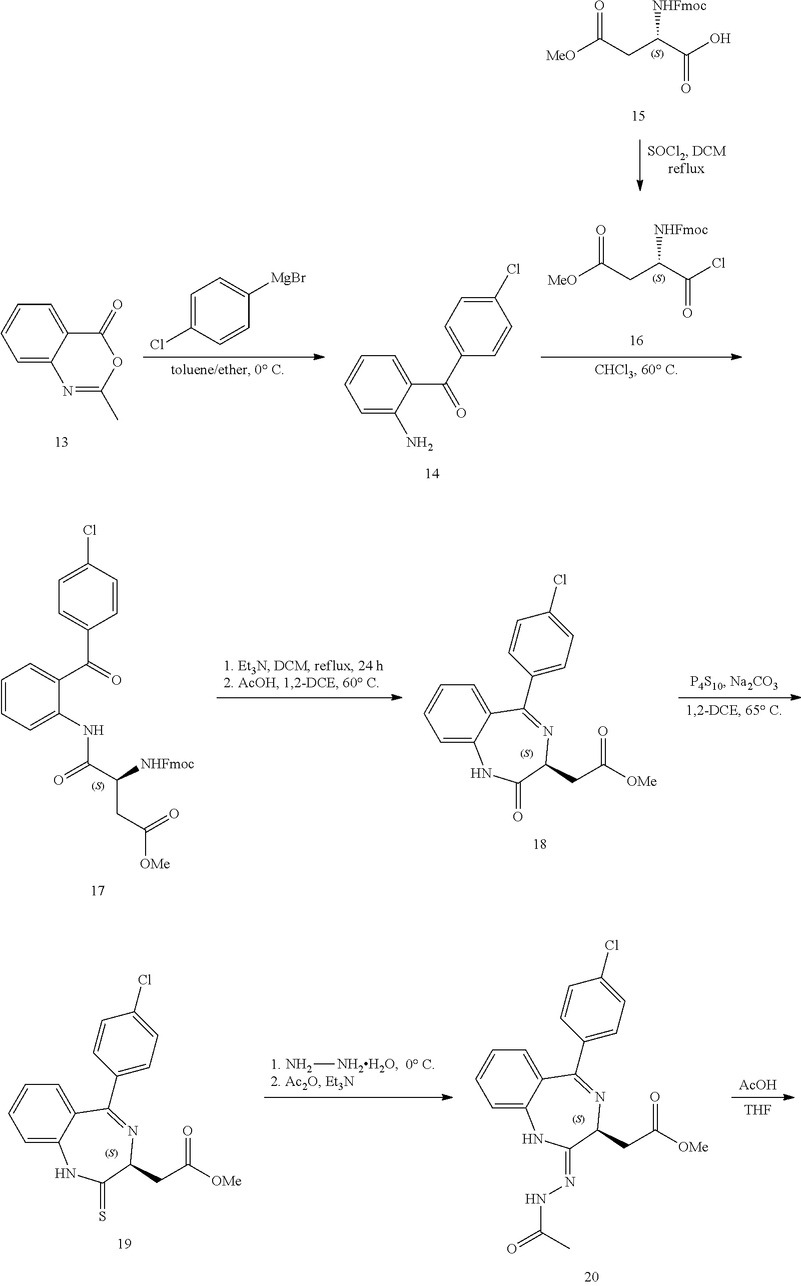

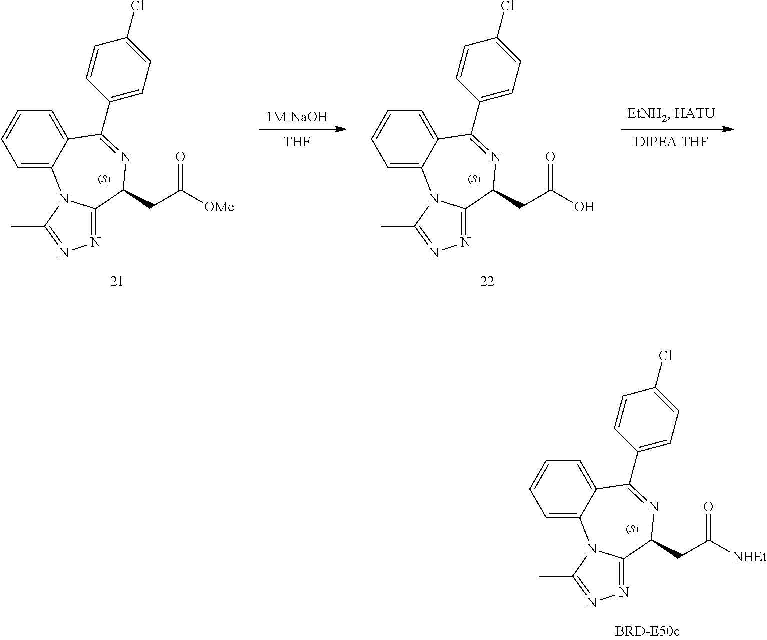

### (2-aminophenyl)(4-chlorophenyl)methanone(14)

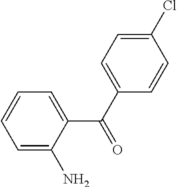 [SMILES: Nc1ccccc1C(=O)c1ccc(Cl)cc1]

Into a 500 mL three-necked round-bottomed flask containing a well-stirred solution of 2-methyl-4H-benzo[d][1,3]oxazin-4-one, 13 (5 g, 31.0 mmol) in a mixture of toluene (100 mL) and diethyl ether (25 mL) was added 4-chlorophenylmagnesium bromide (34.1 mL, 34.1 mmol, 1M in THF) dropwise at 0° C. under nitrogen atmosphere. The reaction mixture was stirred at ambient temperature for 5 h. At that point, the mixture was cooled to 0° C. and quenched by the addition of 1.5N HCl (50 mL), then extracted with ethyl acetate (3×200 mL). The combined organic layers were washed with brine (300 mL), dried over anhydrous sodium sulphate, filtered, and concentrated under reduced pressure. The residue was suspended in a mixture of ethanol (50 mL) and 6N HCl (30 mL), then heated at reflux (80° C.) for 8 h, at which point the mixture was cooled to ambient temperature and concentrated under high vacuum. The resulting residue was suspended in ethyl acetate, neutralized to pH 7 with aqueous 1N NaOH solution, and extracted with ethyl acetate (3×200 mL). The combined organic layers were washed with brine (300 mL), dried over anhydrous sodium sulphate, filtered, and concentrated under reduced pressure. The crude reaction mixture was purified by flash chromatography (60-120 mesh, 20% EtOAc in pet ether) to afford (2-aminophenyl)(4-chlorophenyl)methanone, 2 (6.4 g, 89%) as a yellow solid.

### methyl N-[(9H-fluoren-9-ylmethoxy)carbonyl]-1-α-aspartyl chloride (16)

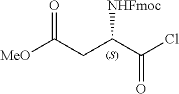

To a stirred solution of Fmoc-Asp-(OMe)-OH, 15 (15 g, 40.6 mmol) in dichloromethane (30 mL) taken was added thionyl chloride (30 mL, 406.1 mmol) dropwise under a nitrogen atmosphere. The reaction mixture was stirred at ambient temperature for 3 h, and then concentrated under reduced pressure. The resulting residue was co-evaporated with toluene (2×20 mL) under a nitrogen atmosphere to afford methyl N-[(9H-fluoren-9-ylmethoxy)carbonyl]-L-α-aspartyl chloride, 16, which was subsequently used without any further purification.

### methyl (S)-3-((((9H-fluoren-9-yl)methoxy)carbonyl)amino)-4-((2-(4-chlorobenzoyl)phenyl)amino)-4-oxobutanoate (17)

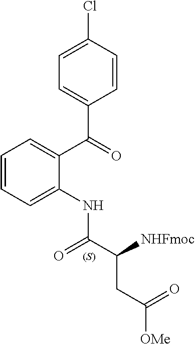

To a stirred 0° C. solution of (2-aminophenyl)(4-chlorophenyl)methanone, 2 (6.4 g, 27.6 mmol) in dry chloroform (80 mL) was added freshly prepared methyl N-[(9H-fluoren-9-ylmethoxy)carbonyl]-L-α-aspartyl chloride, 16, in chloroform (50 mL) under nitrogen atmosphere. The reaction mixture was stirred at ambient temperature for 1 h, then heated at reflux (60° C.) for 3 h. After complete consumption of starting material, the reaction mixture was cooled to ambient temperature and concentrated under reduced pressure. The crude product was co-evaporated with toluene (2×20 mL) to provide methyl (S)-3-((((9H-fluoren-9-yl)methoxy)carbonyl)amino)-4-((2-(4-chlorobenzoyl)phenyl)amino)-4-oxobutanoate, 17 (15 g) which was subsequently used without any further purification.

### methyl (S)-2-(S-(4-chlorophenyl)-2-oxo-2,3-dihydro-1H-benzo[e][1,4]diazepin-3-yl)acetate (18)

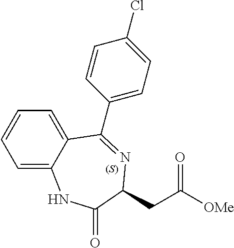

To a stirred solution of methyl (S)-3-((((9H-fluoren-9-yl)methoxy)carbonyl)amino)-4-((2-(4-chlorobenzoyl)phenyl)amino)-4-oxobutanoate, 17 (15 g) in dry dichloromethane (80 mL) was added triethylamine (70 mL) under a nitrogen atmosphere. The mixture was heated at reflux (80° C.) for 5 h, then cooled to ambient temperature and concentrated under reduced pressure. The residue was suspended in dry 1,2-dichloroethane (100 mL) and acetic acid (30 mL) was added. The resulting mixture was heated to 60° C. for 3 h, then cooled to ambient temperature and concentrated under reduced pressure. The resulting residue was dissolved in dichloromethane (500 mL) and sequentially washed with 1.5N HCl (200 mL), water (200 mL), and brine (200 mL). The organic layer was separated, dried over anhydrous sodium sulphate, filtered, and then concentrated under reduced pressure. The crude product was purified by flash chromatography (100-200 mesh, 40% EtOAc in hexane) to afford methyl (S)-2-(5-(4-chlorophenyl)-2-oxo-2,3-dihydro-1H-benzo[e][1,4]diazepin-3-yl)acetate, 18 (3.85 g, 41% over two steps) as a pale yellow solid.

### methyl (S)-2-(5-(4-chlorophenyl)-2-thioxo-2,3-dihydro-1H-benzo[e][1,4]diazepin-3-yl)acetate (19)

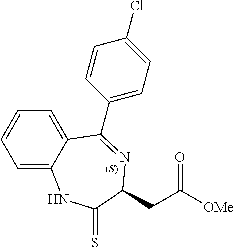

A suspension of phosphorus pentasulfide (9 g, 20.3 mmol) and sodium carbonate (2.14 g, 20.2 mmol) in 1,2-dichloroethane (100 mL) was a stirred at ambient temperature for 1 h, at which point methyl (S)-2-(5-(4-chlorophenyl)-2-oxo-2,3-dihydro-1H-benzo[e][1,4]diazepin-3-yl)acetate, 18 (3.85 g, 11.2 mmol) was added, and the resulting mixture heated at 65° C. for 5 h. The crude reaction mixture was cooled to ambient temperature and filtered through a pad of Celite. The Celite pad was further rinsed with dichloromethane (2×100 mL), and the combined filtrates were washed with saturated aqueous sodium bicarbonate solution (200 mL) and brine (100 mL), then dried over anhydrous sodium sulphate, filtered, and concentrated under reduced pressure. The resulting residue was purified by flash chromatography (100-200 mesh, 30-40% EtOAc in pet ether) to provide methyl (S)-2-(5-(4-chlorophenyl)-2-thioxo-2,3-dihydro-1H-benzo[e][1,4]diazepin-3-yl)acetate, 19 (2.0 g, 50%) as a pale yellow solid.

### methyl (S,Z)-2-(2-(2-acetylhydrazineylidene)-5-(4-chlorophenyl)-2,3-dihydro-JH-benzo[e][1,4]diazepin-3-yl)acetate (20)

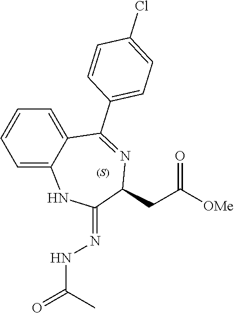

To a well-stirred, 0° C. solution of methyl (S)-2-(5-(4-chlorophenyl)-2-thioxo-2,3-dihydro-1H-benzo[e][1,4]diazepin-3-yl)acetate, 19 (2 g, 5.57 mmol) in dry THF (50 mL) was added hydrazine monohydrate (0.82 mL, 16.7 mmol) under an atmosphere of nitrogen. The mixture was warmed to ambient temperature and stirred for 4 h at which time it was recooled to 0° C. and charged with triethylamine (2.3 mL, 16.8 mmol), then acetyl chloride (1.2 mL, 16.82 mmol). The resulting solution was warmed to ambient temperature and stirred 2 h, at which point the solvents were evaporated. The remaining residue was diluted with water (250 mL) and extracted with dichloromethane (3×200 mL). The combined organic layers were washed with brine (200 mL), dried over anhydrous sodium sulphate, filtered, and concentrated to obtain methyl (S,Z)-2-(2-(2-acetylhydrazineylidene)-5-(4-chlorophenyl)-2,3-dihydro-1H-benzo[e][1,4]diazepin-3-yl)acetate, 20 (2.2 g, 98% over two steps) as a pale yellow solid, which was taken on without any further purification.

### methyl 2-((4S)-6-(4-chlorophenyl)-1-methyl-4H-benzo[f][1,2,4]triazolo[4,3-a][1,4]diazepin-4-yl)acetate (21)

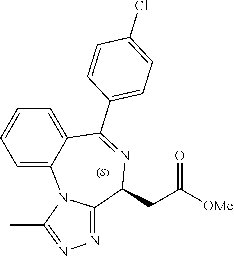

To a well-stirred, 0° C. solution of methyl (S,Z)-2-(2-(2-acetylhydrazineylidene)-5-(4-chlorophenyl)-2,3-dihydro-1H-benzo[e][1,4]diazepin-3-yl)acetate, 20 (2.2 g, 5.13 mmol) in dry THE (50 mL) was added acetic acid (25 mL) under an atmosphere of nitrogen. The reaction mixture was stirred at ambient temperature for 18 h, and was then concentrated under reduced pressure, diluted with water (200 mL) and extracted with dichloromethane (3×200 mL). The combined organic layers were washed with brine (200 mL), dried over anhydrous sodium sulphate, filtered, and concentrated under reduced pressure. The product was purified by flash chromatography (230-500 mesh, 3% MeOH in DCM) to afford methyl 2-((4S)-6-(4-chlorophenyl)-1-methyl-4H-benzo[f][1,2,4]triazolo[4,3-a][1,4]diazepin-4-yl)acetate, 21 (1.9 g, 97%) as a pale yellow solid.

### 2-((4S)-6-(4-chlorophenyl)-1-methyl-4H-benzo[f][1,2,4]triazolo[4,3-a][1,4]diazepin-4-yl)acetic acid (22)

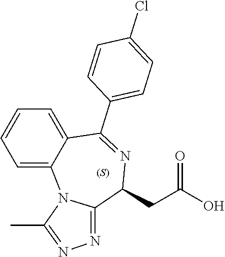

To a stirred, 0° C. solution of methyl 2-((4S)-6-(4-chlorophenyl)-1-methyl-4H-benzo[f][1,2,4]triazolo[4,3-a][1,4]diazepin-4-yl)acetate, 21 (1.9 g, 4.99 mmol) in dry THF (40 mL) was added aqueous 1N NaOH (9.98 mL, 9.98 mmol). The resulting mixture was warmed to ambient temperature and stirred 5 h, and was then concentrated under reduced pressure, diluted with water (200 mL), and washed with EtOAc (250 mL). The aqueous layer was cooled to 0° C. and acidified to pH 3-4 by the addition of 1.5N HCl. The resulting precipitate was filtered and washed with pet ether, then dried under high vacuum to obtain 2-((4S)-6-(4-chlorophenyl)-1-methyl-4H-benzo[f][1,2,4]triazolo[4,3-a][1,4]diazepin-4-yl)acetic acid, 22 (1.2 g, 66%) as a white solid.

### 2-((4S)-6-(4-chlorophenyl)-1-methyl-4H-benzo[f][1,2,4]triazolo[4,3-a][1,4]diazepin-4-yl)-N-ethylacetamide (BRD-E50c)

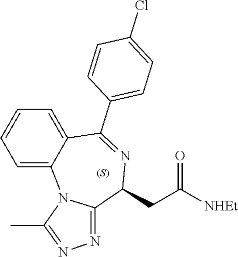

To a stirred, 0° C. solution of 2-((4S)-6-(4-chlorophenyl)-1-methyl-4H-benzo[f][1,2,4]triazolo[4,3-a][1,4]diazepin-4-yl)acetic acid, 22 (250 mg, 0.682 mmol) in dry THE (10 mL) was added DIPEA (230 μL, 1.36 mmol) and HATU (581 mg, 1.36 mmol) under an atmosphere of nitrogen. The resulting mixture was warmed to ambient temperature and stirred for 1 h, at which point ethylamine (1.02 mL, 2M solution in THF, 2.04 mmol) was added. The mixture continued to stir at ambient temperature for 18 h, then was concentrated under reduced pressure, diluted with water (100 mL), and extracted with dichloromethane (3×100 mL). The combined organic layers were washed with brine (100 mL), dried over anhydrous sodium sulphate, filtered, and concentrated under reduced pressure. The product was purified by flash chromatography (230-400 mesh, 10% MeOH in DCM) to afford 2-((4S)-6-(4-chlorophenyl)-1-methyl-4H-benzo[f][1,2,4]triazolo[4,3-a][1,4]diazepin-4-yl)-N-ethylacetamide, BRD-E50c (180 mg, 67%) as a pale brown solid. ¹H-NMR (400 MHz, CD₃OD): δ 7.87-7.80 (m, 2H), 7.64-7.60 (m, 1H), 7.55-7.49 (m, 3H), 7.44-7.41 (m, 2H), 4.65-4.60 (m, 1H), 3.45-3.24 (m, 4H), 2.68 (s, 3H), 1.20 (t, J=7.2 Hz, 3H). LRMS m/z: calcd for C₂₁H₂₀ClN₅O [M+H]⁺: 394.1; found 394.2.

### Synthesis of BRD-E52 Series

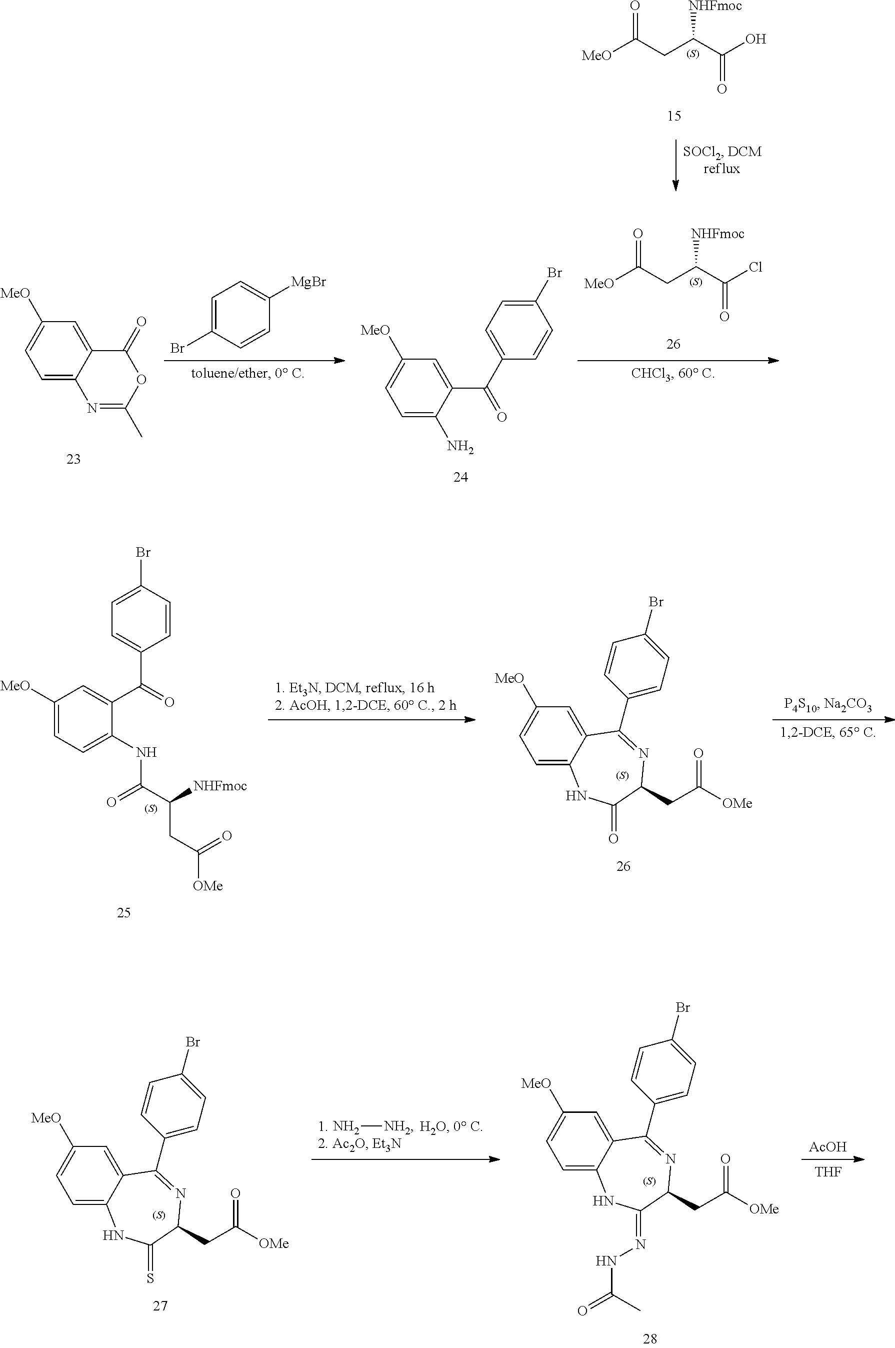

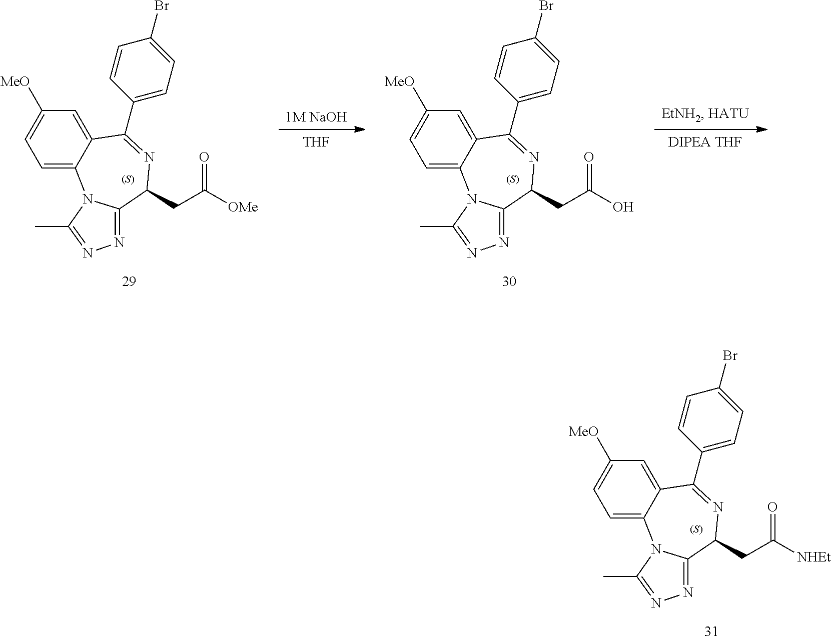

### (2-amino-5-methoxyphenyl)(4-bromophenyl)methanone (24)

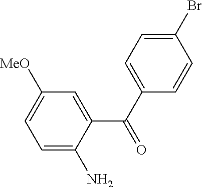 [SMILES: COc1ccc(N)c(C(=O)c2ccc(Br)cc2)c1]

Into a 2 L three-necked round-bottomed flask containing a well-stirred solution of 6-methoxy-2-methyl-4H-benzo[d][1,3]oxazin-4-one, 23 (32 g, 167.4 mmol) in toluene (400 mL) and diethyl ether (100 mL) at 0° C. was added 4-bromophenylmagnesium bromide (268 mL, 0.5M in diethyl ether, 133.9 mmol) under nitrogen atmosphere. The reaction mixture was slowly warmed to ambient temperature over 3 h. At that point, the mixture was cooled to 0° C. and quenched by the addition of 1.5N HCl (100 mL), then extracted with ethyl acetate (3×100 mL). The combined organic layers were washed with brine (100 mL), dried over anhydrous sodium sulphate, filtered, and concentrated under reduced pressure. The residue was suspended in ethanol (100 mL) and 6N HCl (100 mL), then heated at refluxed (80° C.) for 8 h, at which point the mixture was cooled to ambient temperature and concentrated under high vacuum. The pH of the residue was adjusted to 7 using aqueous 1N NaOH and extracted with ethyl acetate (3×200 mL). The combined organic layers were washed with brine (100 mL), dried over anhydrous sodium sulphate, filtered, and concentrated under reduced pressure. The crude reaction mixture was purified by flash chromatography (60-120 mesh, 5-10% EtOAc in pet ether) to afford (2-amino-5-methoxyphenyl)(4-bromophenyl)methanone, 24 (7.5 g, 14.6%) as a yellow solid.

### methyl (S)-3-((((9H-fluoren-9-yl)methoxy)carbonyl)amino)-4-((2-(4-bromobenzoyl)-4-methoxyphenyl)amino)-4-oxobutanoate (25)

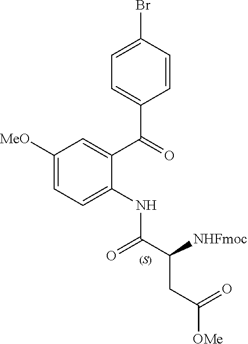

To a stirred 0° C. solution of ((2-amino-5-methoxyphenyl)(4-bromophenyl)methanone, 24 (7.5 g, 24.4 mmol) in dry dichloromethane (50 mL) was added freshly prepared methyl N-[(9H-fluoren-9-ylmethoxy)carbonyl]-L-α-aspartyl chloride, 16, in dichloromethane (50 mL) under nitrogen atmosphere. The reaction mixture slowly warmed to ambient temperature and then heated at reflux (60° C.) for 2 h. After complete consumption of starting material, the reaction mixture was cooled to ambient temperature and concentrated under reduced pressure. The crude product was co-evaporated with toluene (2×20 mL) to provide methyl (S)-3-((((9H-fluoren-9-yl)methoxy)carbonyl)amino)-4-((2-(4-bromobenzoyl)-4-methoxyphenyl)amino)-4-oxobutanoate, 25 (20 g) which was subsequently used without any further purification.

### methyl (S)-2-(5-(4-bromophenyl)-7-methoxy-2-oxo-2,3-dihydro-1H-benzo[e][1,4]diazepin-3-yl)acetate (26)

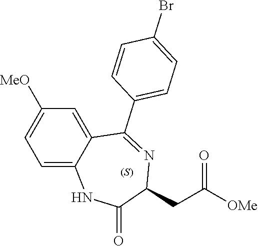

To a stirred solution of methyl (S)-3-((((9H-fluoren-9-yl)methoxy)carbonyl)amino)-4-((2-(4-bromobenzoyl)-4-methoxyphenyl)amino)-4-oxobutanoate, (20 g, 30.4 mmol) in dry dichloromethane (200 mL) was added triethylamine (77 mL, 547.5 mmol) under a nitrogen atmosphere. The mixture was heated at reflux (80° C.) for 18 h, then cooled to ambient temperature and concentrated under reduced pressure. The residue was suspended in dry 1,2-dichloroethane (175 mL) and acetic acid (17 mL, 307.2) was added. The resulting mixture was heated to 60° C. for 2 h, then cooled to ambient temperature and concentrated under reduced pressure. The resulting residue was dissolved in dichloromethane (500 mL) and sequentially washed with 1.5N HCl (100 mL), water (100 mL), and brine (100 mL). The organic layer was separated, dried over anhydrous sodium sulphate, filtered, and then concentrated under reduced pressure. The crude product was suspended in acetonitrile (50 mL) and stirred at ambient temperature for 1 h, at which point the product had precipitated. The precipitate was filtered and dried under high vacuum to afford methyl (S)-2-(5-(4-bromophenyl)-7-methoxy-2-oxo-2,3-dihydro-1H-benzo[e][1,4]diazepin-3-yl)acetate, 26 (5.5 g, 43% over two steps) as a pale yellow solid.

### methyl (S)-2-(5-(4-bromophenyl)-7-methoxy-2-thioxo-2,3-dihydro-1H-benzo[e][1,4]diazepin-3-yl)acetate (27)

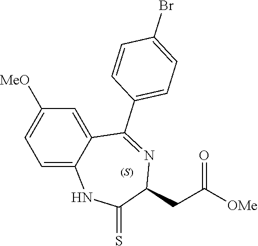

A suspension of phosphorus pentasulfide (10.48 g, 23.6 mmol) and sodium carbonate (2.5 g, 23.6 mmol) in 1,2-dichloroethane (140 mL) was a stirred at ambient temperature for 1 h, at which point methyl (S)-2-(5-(4-bromophenyl)-7-methoxy-2-oxo-2,3-dihydro-1H-benzo[e][1,4]diazepin-3-yl)acetate, 26 (5.5 g, 22.8 mmol) was added, and the resulting mixture heated at 65° C. for 5 h. The reaction mixture was cooled to ambient temperature and filtered through a pad of Celite. The Celite pad was further rinsed with dichloromethane (3×100 mL), and the combined filtrates were washed with saturated aqueous sodium bicarbonate solution (200 mL) and brine (100 mL), then dried over anhydrous sodium sulphate, filtered, and concentrated under reduced pressure. The resulting residue was purified by flash chromatography (60-120 mesh, 30-40% EtOAc in pet ether) to provide methyl (S)-2-(5-(4-bromophenyl)-7-methoxy-2-thioxo-2,3-dihydro-1H-benzo[e][1,4]diazepin-3-yl)acetate, 27 (3.6 g, 63%) as a pale yellow solid.

### methyl (S,Z)-2-(2-(2-acetylhydrazineylidene)-5-(4-bromophenyl)-7-methoxy-2,3-dihydro-1H-benzo[e][1,4]diazepin-3-yl)acetate (28)

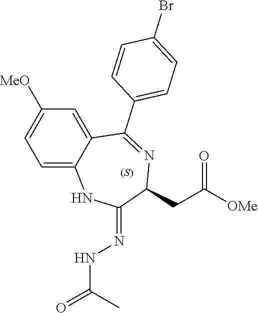

To a well-stirred, 0° C. solution of methyl (S)-2-(5-(4-bromophenyl)-7-methoxy-2-thioxo-2,3-dihydro-1H-benzo[e][1,4]diazepin-3-yl)acetate, 27 (3.6 g, 8.3 mmol) in dry THF (40 mL) was added hydrazine monohydrate (1.9 mL, 26.5 mmol) under an atmosphere of nitrogen. The mixture was warmed to ambient temperature and stirred for 4 h at which time it was recooled to 0° C. and charged with triethylamine (4 mL, 28.2 mmol), then acetyl chloride (1.2 mL, 16.8 mmol). The resulting solution was warmed to ambient temperature and stirred 1 h, at which point the solvents were evaporated. The remaining residue was diluted with water (50 mL) and extracted with dichloromethane (3×50 mL). The combined organic layers were washed with brine (50 mL), dried over anhydrous sodium sulphate, filtered, and concentrated to obtain methyl (S,Z)-2-(2-(2-acetylhydrazineylidene)-5-(4-bromophenyl)-7-methoxy-2,3-dihydro-1H-benzo[e][1,4]diazepin-3-yl)acetate, 28 (3.6 g) as a brown solid, which was taken on without any further purification.

### methyl 2-((4S)-6-(4-bromophenyl)-8-methoxy-1-methyl-4H-benzo[f][1,2,4]triazolo[4,3-a][1,4]diazepin-4-yl)acetate (29)

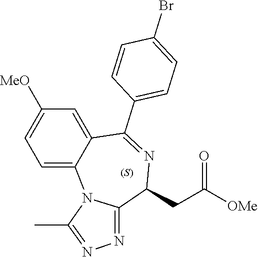

To a well-stirred, 0° C. solution of methyl (S,Z)-2-(2-(2-acetylhydrazineylidene)-5-(4-bromophenyl)-7-methoxy-2,3-dihydro-1H-benzo[e][1,4]diazepin-3-yl)acetate, 28 (3.6 g, 7.6 mmol) in dry THF (40 mL) was added acetic acid (20 mL) under an atmosphere of nitrogen. The reaction stirred at ambient temperature for 18 h, and was then concentrated under reduced pressure, diluted with water (20 mL) and extracted with dichloromethane (3×50 mL). The combined organic layers were washed with brine (100 mL), dried over anhydrous sodium sulphate, filtered, and concentrated under reduced pressure. The product was purified by flash chromatography (60-120 mesh, 2-5% MeOH in DCM) to afford methyl 2-((4S)-6-(4-bromophenyl)-8-methoxy-1-methyl-4H-benzo[f1,2,4]triazolo[4,3-a][1,4]diazepin-4-yl)acetate, 29 (3.3 g, 95%) as a pale yellow solid.

### 2-((4S)-6-(4-bromophenyl)-8-methoxy-1-methyl-4H-benzo[f][1,2,4]triazolo[4,3-a][1,4]diazepin-4-yl)acetic acid (30)

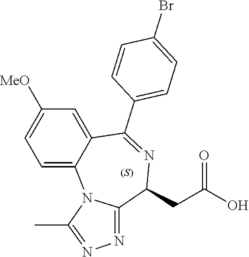

To a stirred, 0° C. solution of methyl 2-((4S)-6-(4-bromophenyl)-8-methoxy-1-methyl-4H-benzo[f][1,2,4]triazolo[4,3-a][1,4]diazepin-4-yl)acetate, 29 (3.3 g, 7.2 mmol) in dry THF (50 mL) was added aqueous 1N NaOH (14.5 mL, 14.5 mmol). The resulting mixture was warmed to ambient temperature and stirred 4 h, and was then concentrated under reduced pressure, diluted with water (200 mL), and washed with EtOAc (250 mL). The aqueous layer was cooled to 0° C. and acidified to pH 3-4 by the addition of 1.5N HCl. The resulting precipitate was filtered and dried under high vacuum to obtain 2-((4S)-6-(4-bromophenyl)-8-methoxy-1-methyl-4H-benzo[f][1,2,4]triazolo[4,3-a][1,4]diazepin-4-yl)acetic acid, 30 (2.4 g, 75%) as a pale brown white solid.

### 2-((4S)-6-(4-bromophenyl)-8-methoxy-1-methyl-4H-benzo[f][1,2,4]triazolo[4,3-a][1,4]diazepin-4-yl)-N-ethylacetamide (31)

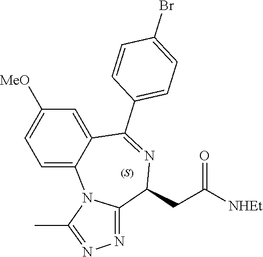

To a well-stirred, 0° C. solution of 2-((4S)-6-(4-bromophenyl)-8-methoxy-1-methyl-4H-benzo[f][1,2,4]triazolo[4,3-a][1,4]diazepin-4-yl)acetic acid, 30 (2.2 g, 4.98 mmol) in dry THF (40 mL) was added DIPEA (1.8 mL, 9.97 mmol) and HATU (3.79 g, 9.97 mmol) under an atmosphere of nitrogen. The resulting mixture was warmed to ambient temperature and stirred for 3 h, at which point ethylamine (4.98 mL, 2M solution in THF, 9.97 mmol) was added. The mixture continued to stir at ambient temperature for 18 h, then was concentrated under reduced pressure, diluted with water (50 mL), and extracted with dichloromethane (3×50 mL). The combined organic layers were washed with brine (100 mL), dried over anhydrous sodium sulphate, filtered, and concentrated under reduced pressure. The product was purified by flash chromatography (60-120 mesh, 2-5% MeOH in DCM) to afford 2-((4S)-6-(4-bromophenyl)-8-methoxy-1-methyl-4H-benzo[f][1,2,4]triazolo[4,3-a][1,4]diazepin-4-yl)-N-ethylacetamide, 31 (2.3 g, 98%) as a pale brown solid.

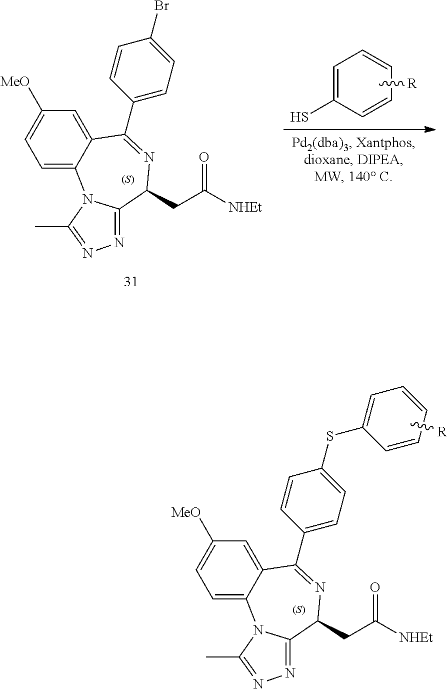

### (3-((4-((4S)-4-(2-(ethylamino)-2-oxoethyl)-8-methoxy-1-methyl-4H-benzo[f][1,2,4]triazolo[4,3-a][1,4]diazepin-6-yl)phenyl)thio)phenyl)boronic acid (BRD-E52)

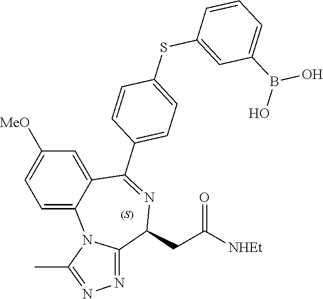

Into an 8 mL microwave reaction vial containing a solution of 2-((4S)-6-(4-bromophenyl)-8-methoxy-1-methyl-4H-benzo[f][1,2,4]triazolo[4,3-a][1,4]diazepin-4-yl)-N-ethylacetamide, 31 (80 mg, 0.17 mmol) in 1,4-dioxane (3 mL) was added 3-mercaptophenylboronic acid (57 mg, 0.34 mmol) followed by DIPEA (0.11 mL, 0.51 mmol). The resulting mixture purged with nitrogen gas for 10 min, at which point Xantphos (10 mg, 34 μmol) and Pd₂(dba)₃ (15 mg, 17 μmol) were added, under a nitrogen atmosphere. The reaction vial was heated to 140° C. under microwave irradiation and stirred for 30 min, at which point the mixture was cooled to ambient temperature the solvent was evaporated under reduced pressure. The product was purified by preparative HPLC (column: X-Select C18 (19×150 mm, 5 μm); mobile phase A: 0.1% formic acid in water; mobile phase B: ACN; flowrate: 15 mL/min). Fractions containing the product were combined and lyophilized to give (3-((4-((4S)-4-(2-(ethylamino)-2-oxoethyl)-8-methoxy-1-methyl-4H-benzo[f][1,2,4]triazolo[4,3-a][1,4]diazepin-6-yl)phenyl)thio)phenyl)boronic acid, BRD-E52 (20 mg, 22%) as an off white solid. ¹H-NMR (400 MHz, CD₃OD): δ 8.35 (brs, 1H), 7.71 (m, 2H), 7.63 (d, J 6.8 Hz, 1H), 7.54-7.36 (m, 6 H), 7.20 (d, J 8.0 Hz, 2H), 6.94 (d, J=2.8 Hz, 1H), 4.62 (q, J=5.2 Hz, 1H), 3.84 (s, 3H), 3.42-3.33 (m, 2H), 3.29-3.21 (m, 3H), 2.54 (s, 3H), 1.18 (t, J=7.2 Hz, 3H). LRMS m/z: calcd for C₂₈H₂₈BN₅O₄S [M+H]⁺: 542.2; found 542.2.

### N-ethyl-2-((4S)-8-methoxy-1-methyl-6-(4-(phenylthio)phenyl)-4H-benzo[f][1,2,4]triazolo[4,3-a][1,4]diazepin-4-yl)acetamide (BRD E52c)

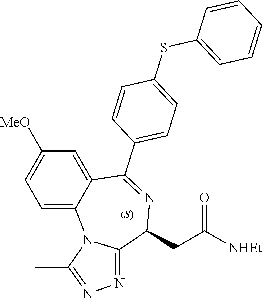

Into an 8 mL microwave reaction vial containing a solution of 2-((4S)-6-(4-bromophenyl)-8-methoxy-1-methyl-4H-benzo[f][1,2,4]triazolo[4,3-a][1,4]diazepin-4-yl)-N-ethylacetamide, 31 (100 mg, 0.21 mmol) in 1,4-dioxane (3 mL) was added thiophenol (65 mg, 0.42 mmol) followed by DIPEA (0.11 mL, 0.64 mmol). The resulting mixture purged with nitrogen gas for 10 min, at which point Xantphos (10 mg, 42 μmol) and Pd₂(dba)₃ (15 mg, 21 μmol) were added, under a nitrogen atmosphere. The reaction vial was heated to 140° C. under microwave irradiation and stirred for 30 min, at which point the mixture was cooled to ambient temperature and the solvent was evaporated under reduced pressure. The product was purified by preparative HPLC (column: X-Select C18 (19×150 mm, 5 μm); mobile phase A: 0.1% formic acid in water; mobile phase B: ACN; flowrate: 15 mL/min). Fractions containing the product were combined and lyophilized to give N-ethyl-2-((4S)-8-methoxy-1-methyl-6-(4-(phenylthio)phenyl)-4H-benzo[f][1,2,4]triazolo[4,3-a][1,4]diazepin-4-yl)acetamide, BRD-E52c (50 mg, 47%) as an off white solid. ¹H-NMR (400 MHz, CD₃OD): δ 8.52 (brs, 1H), 7.72 (d, J=8.8 Hz, 1H), 7.48-7.46 (m, 4H), 7.44-7.36 (m, 4H), 7.22-7.19 (m, 2H), 6.95 (d, J=3.2 Hz, 1H), 4.62 (q, J=5.2 Hz, 1H), 3.84 (s, 3H), 3.42-3.33 (m, 2H), 3.23-3.21 (m, 2H), 2.64 (s, 3H), 1.18 (t, J=7.2 Hz, 3H); LRMS m/z: calcd for C₂₈H₂₇N₅O₂S [M+H]⁺: 498.2; found 498.4.

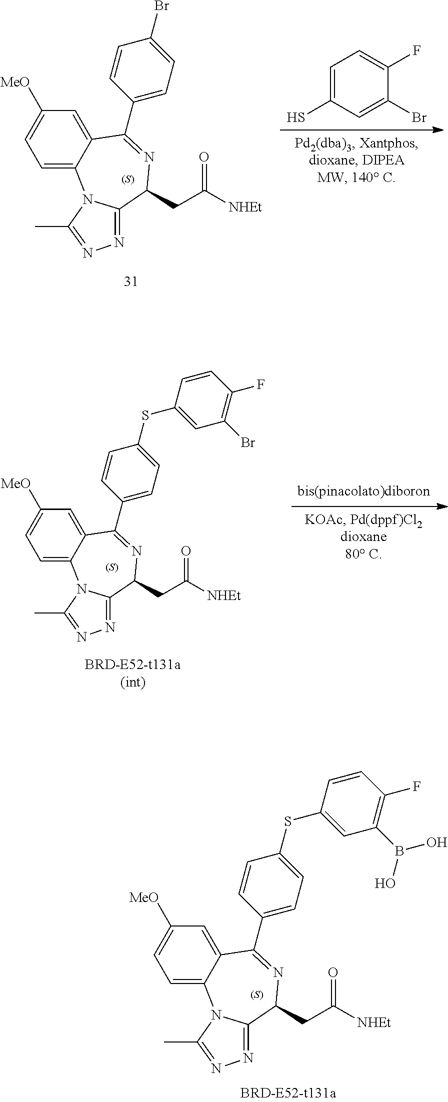

### 2-((4S)-6-(4-((3-bromo-4-fluorophenyl)thio)phenyl)-8-methoxy-1-methyl-4H-benzo[f][1,2,4]triazolo[4,3-a][1,4]diazepin-4-yl)-N-ethylacetamide (BRD-E52-t131a (int))

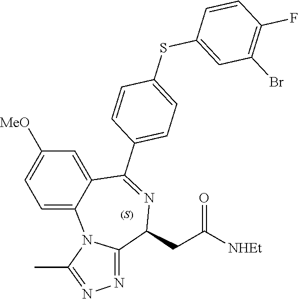

Into an 8 mL microwave reaction vial containing a solution of 2-((4S)-6-(4-bromophenyl)-8-methoxy-1-methyl-4H-benzo[f][1,2,4]triazolo[4,3-a][1,4]diazepin-4-yl)-N-ethylacetamide, 31 (400 mg, 0.85 mmol) in 1,4-dioxane (3 mL) was added 3-bromo-4-fluorobenzenethiol (353 mg, 1.7 mmol) followed by DIPEA (0.46 mL, 2.56 mmol). The resulting mixture purged with nitrogen gas for 10 min, at which point Xantphos (98.8 mg, 170 μmol) and Pd₂(dba)₃ (78 mg, 85 μmol) were added, under a nitrogen atmosphere. The reaction vial was heated to 140° C. under microwave irradiation and stirred for 30 min, at which point the mixture was cooled to ambient temperature and the solvent was evaporated under reduced pressure. The product was purified by preparative HPLC (column: X-Select C18 (19×150 mm, 5 μm); mobile phase A: 0.1% formic acid in water; mobile phase B: ACN; flowrate: 15 mL/min). Fractions containing the product were combined and lyophilized to give 2-((4S)-6-(4-((3-bromo-4-fluorophenyl)thio)phenyl)-8-methoxy-1-methyl-4H-benzo[f][1,2,4]triazolo[4,3-a][1,4]diazepin-4-yl)-N-ethylacetamide, BRD-E52c-t131a (int) (100 mg, 19.7%) as an off white solid.

### (5-((4-((4S)-4-(2-(ethylamino)-2-oxoethyl)-8-methoxy-1-methyl-4H-benzo[f][1,2,4]triazolo[4,3-a][1,4]diazepin-6-yl)phenyl)thio)-2-fluorophenyl)boronic acid (BRD-E52-t131a)

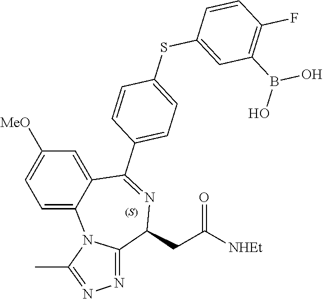

To an 8 mL microwave reaction vial containing a solution of 2-((4S)-6-(4-((3-bromo-4-fluorophenyl)thio)phenyl)-8-methoxy-1-methyl-4H-benzo[f][1,2,4]triazolo[4,3-a][1,4]diazepin-4-yl)-N-ethylacetamide, BRD-E52c-t131a (int) (100 mg, 0.17 mmol) in 1,4-dioxane (3 mL) was added bis(pinacolato)diboron (215 mg, 0.84 mmol) and potassium acetate (50 mg, 0.50 mmol). The resulting solution was purged with nitrogen for 10 min, at which point Pd(dppf)Cl₂·DCM (14 mg, 16.8 μmol) was added and the resulting mixture was heated at 140° C. under microwave irradiation for 30 min, then cooled to ambient temperature and concentrated under reduced pressure. The resulting mixture was purified by preparative HPLC [column: X-Select C18 (19×150 mm, 5 μm); mobile phase A: 0.1% formic acid in water; mobile phase B: ACN; flowrate: 15 mL/min]. Fractions containing the product were combined and lyophilized to afford (5-((4-((4S)-4-(2-(ethylamino)-2-oxoethyl)-8-methoxy-1-methyl-4H-benzo[f][1,2,4]triazolo[4,3-a][1,4]diazepin-6-yl)phenyl)thio)-2-fluorophenyl)boronic acid, BRD-E52-t131a (20 mg, 21%) as a white solid. ¹H-NMR (400 MHz, CD₃OD): δ 7.72 (d, J=8.8 Hz, 1H), 7.58-7.52 (m, 2H), 7.46 (d, J=8.4 Hz, 2H), 7.38 (dd, J=2.8, 8.8 Hz, 1H), 7.17-7.11 (m, 3H), 6.94 (d, J=2.8 Hz, 1H), 4.62 (q, J=5.2 Hz, 1H), 3.84 (s, 3H), 3.42-3.36 (m, 2H), 3.30-3.21 (m, 2H), 2.64 (s, 3H), 1.19 (t, J=7.2 Hz, 3H). LRMS m/z: calcd for C₂₉H₂₇BClN₅O₄S [M+H]⁺: 560.1; found 560.1.

### Common Acid/Phenol Intermediate

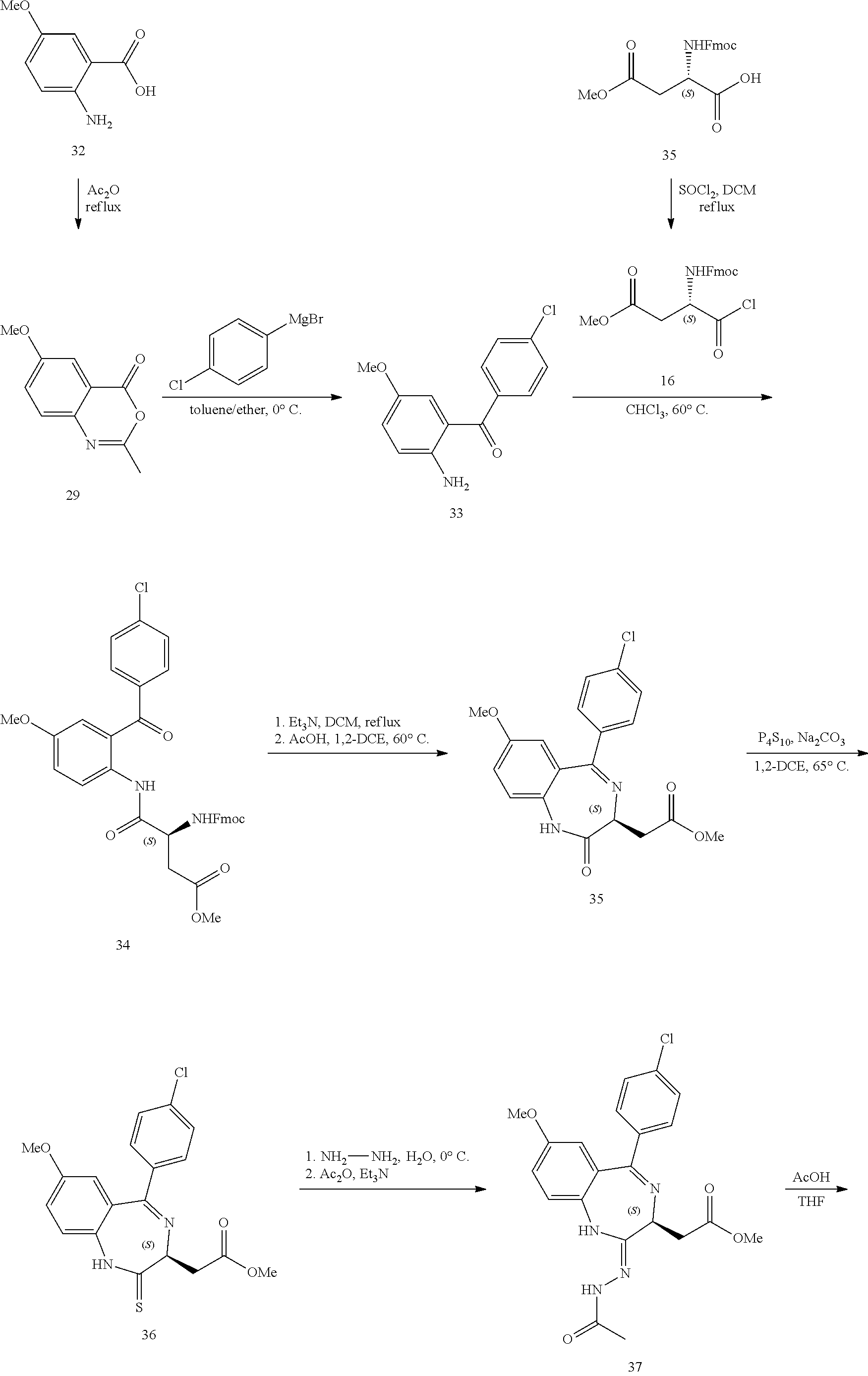

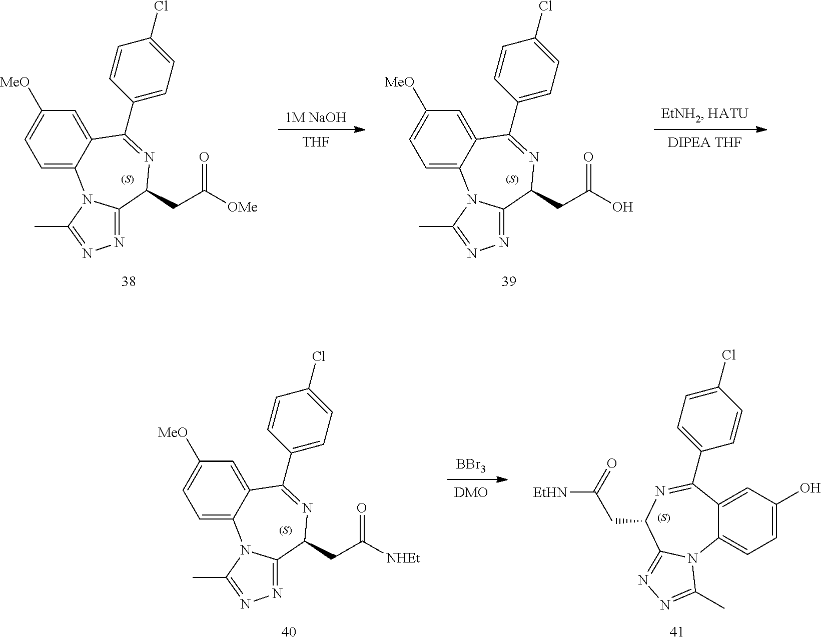

The synthetic approach to the Common Acid/Phenol Intermediate can be found in WO2011054845, to Bailey et al., which is hereby incorporated by reference in its entirety.

### 6-methoxy-2-methyl-4H-benzo[d][1,3]oxazin-4-one (23)

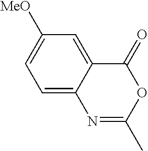

A solution of 5-methoxyanthranilic acid 32 (30 g, 179.5 mmol) in acetic anhydride (300 mL) was heated at reflux (140° C.) for 18 h under a nitrogen atmosphere, and then concentrated under reduced pressure. The remaining residue was triturated with diethyl ether (100 mL) and pet ether (100 mL), and the resulting precipitate filtered to afford 6-methoxy-2-methyl-4H-benzo[d][1,3]oxazin-4-one, 23 (26 g) as a pale brown solid, which was subsequently used without further purification.

### (2-amino-5-methoxyphenyl)(4-chlorophenyl)methanone (33)

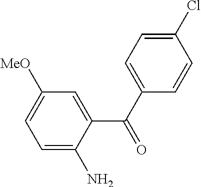 [SMILES: COc1ccc(N)c(C(=O)c2ccc(Cl)cc2)c1]

Into a 1 L three-necked round-bottomed flask containing a well-stirred solution of 6-methoxy-2-methyl-4H-benzo[d][1,3]oxazin-4-one, 23 (13 g, 68.0 mmol) in a mixture of toluene (200 mL) and diethyl ether (50 mL) was added 4-chlorophenylmagnesium bromide (54.2 mL, 54.2 mmol, 1M in THE) dropwise at 0° C. under a nitrogen atmosphere. The reaction mixture was stirred at ambient temperature for 3 h. At that point, the mixture was cooled to 0° C. and quenched by the addition of 1.5N HCl (100 mL), then extracted with ethyl acetate (3×100 mL). The combined organic layers were washed with brine (100 mL), dried over anhydrous sodium sulphate, filtered, and concentrated under reduced pressure. The residue was suspended in a mixture of ethanol (50 mL) and 6N HCl (50 mL), then heated at reflux (80° C.) for 8 h, at which point the mixture was cooled to ambient temperature and concentrated under high vacuum. The resulting residue was suspended in ethyl acetate, neutralized to pH 7 with aqueous 1N NaOH solution, and extracted with ethyl acetate (3×100 mL). The combined organic layers were washed with brine (100 mL), dried over anhydrous sodium sulphate, filtered, and concentrated under reduced pressure. The crude reaction mixture was purified by flash chromatography (60-120 mesh, 5-20% EtOAc in pet ether) to afford 6-methoxy-2-methyl-4H-benzo[d][1,3]oxazin-4-one, 33 (10 g, 56%) as a yellow solid.

### methyl (S)-3-((((9H-fluoren-9-yl)methoxy)carbonyl)amino)-4-((2-(4-chlorobenzoyl)-4-methoxyphenyl)amino)-4-oxobutanoate (34)

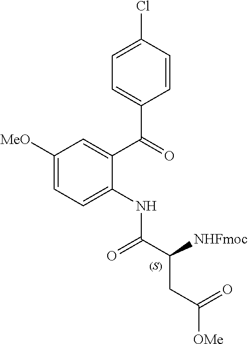

To a stirred 0° C. solution of 6-methoxy-2-methyl-4H-benzo[d][1,3]oxazin-4-one, 33 (10 g, 38.2 mmol) in dry dichloromethane (50 mL) was added freshly prepared methyl N-[(9H-fluoren-9-ylmethoxy)carbonyl]-L-α-aspartyl chloride, 16, in dichloromethane (50 mL) under nitrogen atmosphere. The reaction mixture was warmed to ambient temperature and then heated at reflux (60° C.) for 2 h. After complete consumption of starting material, the reaction mixture was cooled to ambient temperature and concentrated under reduced pressure. The crude product was co-evaporated with toluene (2×20 mL) to provide methyl (S)-3-((((9H-fluoren-9-yl)methoxy)carbonyl)amino)-4-((2-(4-chlorobenzoyl)-4-methoxyphenyl)amino)-4-oxobutanoate, 34 (25 g) which was subsequently used without any further purification.

### methyl (S)-2-(5-(4-chlorophenyl)-7-methoxy-2-oxo-2,3-dihydro-1H-benzo[e][1,4]diazepin-3-yl)acetate (35)

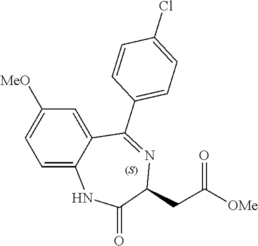

To a stirred solution of methyl (S)-3-((((9H-fluoren-9-yl)methoxy)carbonyl)amino)-4-((2-(4-chlorobenzoyl)-4-methoxyphenyl)amino)-4-oxobutanoate, 34 (25 g, 40.8 mmol) in dry dichloromethane (80 mL) was added triethylamine (103 mL, 734 mmol) under a nitrogen atmosphere. The mixture was heated at reflux (80° C.) for 18 h, then cooled to ambient temperature and concentrated under reduced pressure. The residue was suspended in dry 1,2-dichloroethane (230 mL) and acetic acid (23.3 mL, 408 mmol) was added. The resulting mixture was heated to 60° C. for 2 h, then cooled to ambient temperature and concentrated under reduced pressure. The resulting residue was dissolved in dichloromethane (500 mL) and sequentially washed with 1.5N HCl (100 mL), water (100 mL), and brine (100 mL). The organic layer was separated, dried over anhydrous sodium sulphate, filtered, and then concentrated under reduced pressure. The crude product was suspended in acetonitrile (50 ml) and stirred for 1 h. The resulting precipitate was filtered and dried under high vacuum to afford methyl (S)-2-(5-(4-chlorophenyl)-7-methoxy-2-oxo-2,3-dihydro-1H-benzo[e][1,4]diazepin-3-yl)acetate, 35 (8.5 g, 55.9%) as a pale yellow solid.

### methyl (S)-2-(5-(4-chlorophenyl)-7-methoxy-2-thioxo-2,3-dihydro-JH-benzo[e][1,4]diazepin-3-yl)acetate (36)

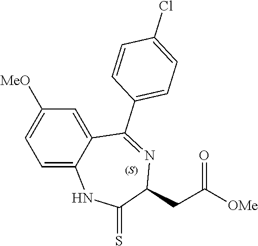

A suspension of phosphorus pentasulfide (18.24 g, 41.0 mmol) and sodium carbonate (4.35 g, 41.0 mmol) in 1,2-dichloroethane (150 mL) was a stirred at ambient temperature for 1 h, at which point methyl (S)-2-(5-(4-chlorophenyl)-7-methoxy-2-oxo-2,3-dihydro-1H-benzo[e][1,4]diazepin-3-yl)acetate, 35 (8.5 g, 22.8 mmol) was added, and the resulting mixture was heated at 65° C. for 5 h. The crude reaction mixture was cooled to ambient temperature and filtered through a pad of Celite. The Celite pad was further rinsed with dichloromethane (2×100 mL), and the combined filtrates were washed with saturated aqueous sodium bicarbonate solution (200 mL) and brine (100 mL), then dried over anhydrous sodium sulphate, filtered, and concentrated under reduced pressure. The resulting residue was purified by flash chromatography (60-120 mesh, 30-40% EtOAc in pet ether) to provide methyl (S)-2-(5-(4-chlorophenyl)-7-methoxy-2-thioxo-2,3-dihydro-1H-benzo[e][1,4]diazepin-3-yl)acetate, 36 (6.0 g, 67.7%) as a pale yellow solid.

### methyl (S,Z)-2-(2-(2-acetylhydrazineylidene)-5-(4-chlorophenyl)-7-methoxy-2,3-dihydro-1H-benzo[e][1,4]diazepin-3-yl)acetate (37)

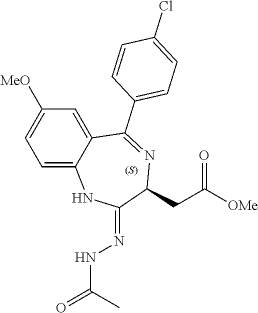

To a well-stirred, 0° C. solution of methyl (S)-2-(5-(4-chlorophenyl)-7-methoxy-2-thioxo-2,3-dihydro-1H-benzo[e][1,4]diazepin-3-yl)acetate, 36 (6.0 g, 15.4 mmol) in dry THE (100 mL) was added hydrazine monohydrate (1.62 mL, 46.3 mmol) under an atmosphere of nitrogen. The mixture was warmed to ambient temperature and stirred for 4 h at which time it was recooled to 0° C. and charged with triethylamine (6.5 mL, 46.3 mmol), then acetyl chloride (3.3 mL, 46.3 mmol). The resulting solution was warmed to ambient temperature and stirred 1 h, at which point the solvents were evaporated. The remaining residue was diluted with water (50 mL) and extracted with dichloromethane (3×100 mL). The combined organic layers were washed with brine (100 mL), dried over anhydrous sodium sulphate, filtered, and concentrated to obtain methyl (S,Z)-2-(2-(2-acetylhydrazineylidene)-5-(4-chlorophenyl)-7-methoxy-2,3-dihydro-1H-benzo[e][1,4]diazepin-3-yl)acetate, 37 (6 g) as a pale yellow solid, which was taken on without any further purification.

### methyl 2-((4S)-6-(4-chlorophenyl)-8-methoxy-1-methyl-4H-benzo[f][1,2,4]triazolo[4,3-a][1,4]diazepin-4-yl)acetate (38)

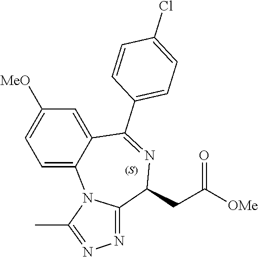

To a well-stirred, 0° C. solution of methyl (S,Z)-2-(2-(2-acetylhydrazineylidene)-5-(4-chlorophenyl)-7-methoxy-2,3-dihydro-1H-benzo[e][1,4]diazepin-3-yl)acetate, 37 (6 g, 14.0 mmol) in dry THF (10 mL) was added acetic acid (50 mL) under an atmosphere of nitrogen. The reaction mixture was stirred at ambient temperature for 18 h, and then concentrated under reduced pressure, diluted with water (50 mL) and extracted with dichloromethane (3×100 mL). The combined organic layers were washed with brine (100 mL), dried over anhydrous sodium sulphate, filtered, and concentrated under reduced pressure. The product was purified by flash chromatography (60-120 mesh, 2-5% MeOH in DCM) to afford methyl 2-((4S)-6-(4-chlorophenyl)-8-methoxy-1-methyl-4H-benzo[1,2,4]triazolo[4,3-a][1,4]diazepin-4-yl)acetate, 38 (3.7 g, 64.4%) as a pale yellow solid.

### 2-((4S)-6-(4-chlorophenyl)-8-methoxy-1-methyl-4H-benzo[f][1,2,4]triazolo[4,3-a][1,4]diazepin-4-yl)acetic acid (39, acid scaffold intermediate)

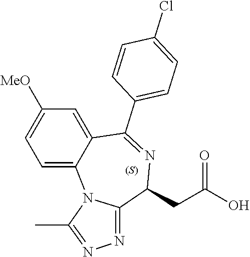

To a stirred, 0° C. solution of methyl 2-((4S)-6-(4-chlorophenyl)-8-methoxy-1-methyl-4H-benzo[f][1,2,4]triazolo[4,3-a][1,4]diazepin-4-yl)acetate, 26 (4.4 g, 10.7 mmol) in dry THF (80 mL) was added aqueous 1N NaOH (21.4 mL, 21.4 mmol). The resulting mixture was warmed to ambient temperature and stirred 4 h, and was then concentrated under reduced pressure, diluted with water (200 mL), and washed with EtOAc (250 mL). The aqueous layer was cooled to 0° C. and acidified to pH 3-4 by the addition of 1.5N HCl. The resulting precipitate was filtered and dried under high vacuum to obtain 2-((4S)-6-(4-chlorophenyl)-8-methoxy-1-methyl-4H-benzo[f][1,2,4]triazolo[4,3-a][1,4]diazepin-4-yl)acetic acid, 39 (3.7 g, 87.3%) as a pale brown solid.

### 2-((4S)-6-(4-chlorophenyl)-8-methoxy-1-methyl-4H-benzo[f][1,2,4]triazolo[4,3-a][1,4]diazepin-4-yl)-N-ethylacetamide (40)

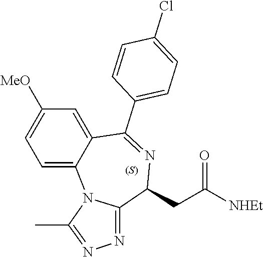

To a stirred, 0° C. solution of 2-((4S)-6-(4-chlorophenyl)-8-methoxy-1-methyl-4H-benzo[f][1,2,4]triazolo[4,3-a][1,4]diazepin-4-yl)acetic acid, 39 (3.7 g, 9.32 mmol) in dry THF (80 mL) was added DIPEA (3.34 mL, 18.6 mmol) and HATU (7.09 g, 18.6 mmol) under an atmosphere of nitrogen. The resulting mixture was warmed to ambient temperature and stirred for 3 h, at which point ethylamine (9.3 mL, 2M solution in THF, 18.6 mmol) was added. The mixture continued to stir at ambient temperature for 18 h, then was concentrated under reduced pressure, diluted with water (50 mL), and extracted with dichloromethane (3×100 mL). The combined organic layers were washed with brine (100 mL), dried over anhydrous sodium sulphate, filtered, and concentrated under reduced pressure. The product was purified by flash chromatography (60-120 mesh, 2-5% MeOH in DCM) to afford 2-((4S)-6-(4-chlorophenyl)-8-methoxy-1-methyl-4H-benzo[f][1,2,4]triazolo[4,3-a][1,4]diazepin-4-yl)-N-ethylacetamide, 40 (2.6 g, 65.8%) as a pale brown solid.

### 2-((4S)-6-(4-chlorophenyl)-8-hydroxy-1-methyl-4H-benzo[f][1,2,4]triazolo[4,3-a][1,4]diazepin-4-yl)-N-ethylacetamide (41, phenol scaffold intermediate)

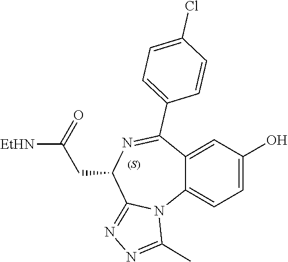

To a stirred, −78° C. solution of 2-((4S)-6-(4-chlorophenyl)-8-methoxy-1-methyl-4H-benzo[f][1,2,4]triazolo[4,3-a][1,4]diazepin-4-yl)-N-ethylacetamide, 40 (1.0 g, 2.36 mmol) in dry dichloromethane (20 mL) was added boron tribromide (9.5 mL, 9.5 mmol, 1M solution in DCM) under a nitrogen atmosphere. The resulting mixture was warmed to ambient temperature and for 4 h, at which point it was cooled to 0° C., quenched with saturated dithionite solution (30 mL), and extracted with ethyl acetate (3×60 mL). The combined organic layers were dried over anhydrous sodium sulphate, filtered, and concentrated under reduced pressure. The product was purified by flash chromatography (60-120 mesh, 8-10% MeOH in DCM) to afford 2-((4S)-6-(4-chlorophenyl)-8-hydroxy-1-methyl-4H-benzo[f][1,2,4]triazolo[4,3-a][1,4]diazepin-4-yl)-N-ethylacetamide, 41 (700 mg, 72.4%) as a pale yellow solid.

### Acid Scaffold Compounds

### General Procedure for EDC Coupling

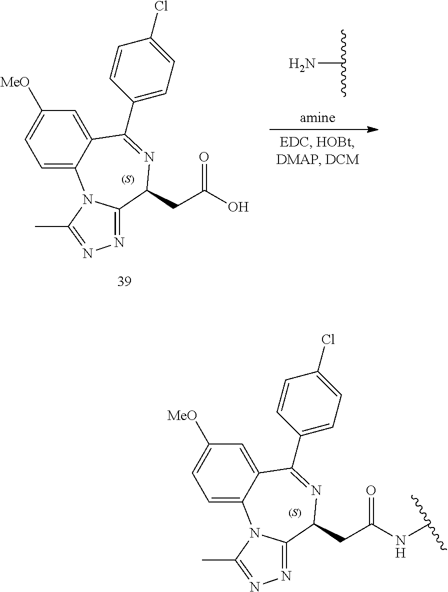

To a stirred solution of 2-((4S)-6-(4-chlorophenyl)-8-methoxy-1-methyl-4H-benzo[f][1,2,4]triazolo[4,3-a][1,4]diazepin-4-yl)acetic acid (39, 0.25 mmol, 1.0 eq.) in dry dichloromethane (DCM, 4 mL) was added 4-(dimethylamino)pyridine (DMAP, 1.5 eq.), N-(3-dimethylaminopropyl)-N′-ethylcarbodiimide hydrochloride (EDC, 1.5 eq.) and 1-hydroxybenzotriazole (HOBt, 1.5 eq.). The resulting mixture was stirred at ambient temperature for 15 min at which point the amine moiety (1.5 eq.) was added and the reaction continued to stir for an additional 18 h. Upon completion, the reaction was diluted with DCM (10 mL) and then washed sequentially with freshly prepared 5% acetic acid in water (5 mL), water (5 mL), and brine (5 mL). The organic layer was separated, dried over anhydrous sodium sulphate, filtered, and concentrated under reduced pressure. The reaction was purified either by preparative HPLC [column: X-Select C18 (19×150 mm, 5 μm); mobile phase A: 0.1% formic acid in water; mobile phase B: ACN; flowrate: 15 mL/min] or by flash chromatography (60-120 mesh, 8-10% MeOH in DCM). Fractions containing the product were combined and lyophilized

### General Procedurefor HATU Coupling

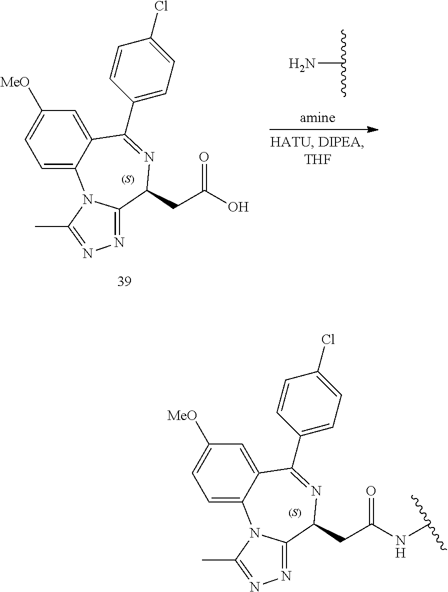

To a well-stirred solution of 2-((4S)-6-(4-chlorophenyl)-8-methoxy-1-methyl-4H-benzo[f][1,2,4]triazolo[4,3-a][1,4]diazepin-4-yl)acetic acid (39, 0.25 mmol, 1.0 eq.) in dry tetrahydrofuran (THF, 4 mL) at ambient temperature was added N,N-diisopropylethylamine (DIPEA, 2 eq.) and 2-(3H-[1,2,3]triazolo[4,5-b]pyridin-3-yl)-1,1,3,3-tetramethylisouronium hexafluorophosphate (HATU, 1.5 eq.) under a nitrogen atmosphere. The resulting mixture was stirred at ambient temperature for 15 min at which point the amine moiety (1.3 eq.) was added. The reaction was heated at 50° C. and stirred for an additional 6 h. Upon completion, the reaction was cooled and diluted with DCM (10 mL) and then washed sequentially with water (5 mL), and brine (5 mL). The organic layer was separated, dried over anhydrous sodium sulphate, filtered, and concentrated under reduced pressure. The reaction was purified either by preparative HPLC [column: X-Select C18 (19×150 mm, 5 m); mobile phase A: 0.1% formic acid in water; mobile phase B: ACN; flowrate: 15 mL/min] or by flash chromatography (60-120 mesh, 8-10% MeOH in DCM). Fractions containing the product were combined and lyophilized.

### (3-(2-((4S)-6-(4-chlorophenyl)-8-methoxy-1-methyl-4H-benzo[f][1,2,4]triazolo[4,3-a][1,4]diazepin-4-yl)acetamido)phenyl)boronic acid (BRD-E73)

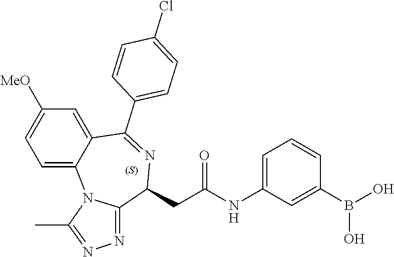

(3-(2-((4S)-6-(4-chlorophenyl)-8-methoxy-1-methyl-4H-benzo[f][1,2,4]triazolo[4,3-a][1,4]diazepin-4-yl)acetamido)phenyl)boronic acid (BRD-E73) was synthesized by following the method of general EDC coupling of 39 (100 mg, 0.25 mmol) and (3-aminophenyl)boronic acid (52 mg, 0.38 mmol). BRD-E73 (70 mg, 53.8%) was isolated by preparative HPLC as an off-white solid. ¹H NMR (400 MHz, CD₃OD): δ 7.84 (s, 1H), 7.79 (d, J=8.8 Hz, 1H), 7.70-7.68 (m, 1H), 7.57 (d, J=6.8 Hz, 2H), 7.44-7.41 (m, 3H), 7.37-7.35 (m, 2H), 6.99 (d, J=2.8 Hz, 1H), 4.79 (dd, J=5.6, 8.8 Hz, 1H), 3.86 (s, 3H), 3.68-3.62 (m, 1H), 3.53-3.51 (m, 1H), 2.75 (s, 3H). LRMS m/z: calcd for C₂₆H₂₃BClN₅O₄ [M+H]⁺: 516.2; found 516.2.

### 2-((4S)-6-(4-chlorophenyl)-8-methoxy-1-methyl-4H-benzo[f][1,2,4]triazolo[4,3-a][1,4]diazepin-4-yl)-N-(3-hydroxyphenyl)acetamide (BRD-E73c)

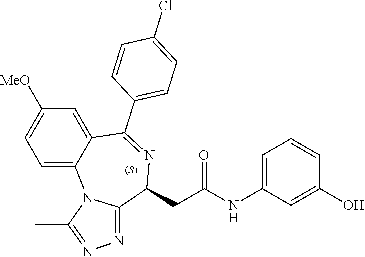

2-((4S)-6-(4-chlorophenyl)-8-methoxy-1-methyl-4H-benzo[f][1,2,4]triazolo[4,3-a][1,4]diazepin-4-yl)-N-(3-hydroxyphenyl)acetamide (BRD-E73c) was synthesized by following the method of general EDC coupling of 39 (100 mg, 0.25 mmol) and 3-aminophenol (41 mg, 0.38 mmol). BRD-E73c (35 mg, 28.4%) was isolated by flash chromatography as a pale yellow solid. ¹H-NMR (400 MHz, DMSO-d₆): δ 10.19 (s, 1H), 9.36 (s, 1H), 7.81 (d, J=8.8 Hz, 1H), 7.50 (dd, J=2.4, 12.4 Hz, 4H), 7.40 (dd, J=2.8, 9.2 Hz, 1H), 7.21 (t, J=2.0 Hz, 1H), 7.08 (t, J=8.0 Hz, 1H), 7.03 (d, J=8.4 Hz, 1H), 6.90 (d, J=2.8 Hz, 1H), 6.45 (d, J=1.2 Hz, 1H), 4.58-4.54 (m, 1H), 3.80 (s, 3H), 3.54-3.48 (m, 1H), 3.43-3.40 (m, 1H), 2.55 (s, 3H). LRMS m/z: calcd for C₂₆H₂₂ClN₅O₃ [M+H]⁺: 488.1; found 488.2.

### (4-(2-((4S)-6-(4-chlorophenyl)-8-methoxy-1-methyl-4H-benzo[f][1,2,4]triazolo[4,3-a][1,4]diazepin-4-yl)acetamido)phenyl)boronic acid (BRD-E74)

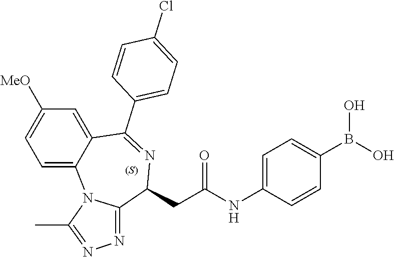

(4-(2-((4S)-6-(4-chlorophenyl)-8-methoxy-1-methyl-4H-benzo[f][1,2,4]triazolo[4,3-a][1,4]diazepin-4-yl)acetamido)phenyl)boronic acid (BRD-E74) was synthesized by following the method of general EDC coupling of 39 (100 mg, 0.25 mmol) and (4-aminophenyl)boronic acid (52 mg, 0.38 mmol). BRD-E74 (20 mg, 15.4%) was isolated by preparative HPLC as an off-white solid. ¹H-NMR (400 MHz, CD₃OD): δ 8.44 (s, 1H), 7.75 (d, J=8.8 Hz, 1H), 7.61 (s, 3H), 7.56 (d, J=8.4 Hz, 2H), 7.42-7.39 (m, 3H), 6.96 (d, J=2.8 Hz, 1H), 4.74 (q, J=5.2 Hz, 1H), 3.85 (s, 3H), 3.68-3.62 (m, 1H), 3.51-3.46 (m, 1H), 2.67 (s, 3H). LRMS m/z: calcd for C₂₆H₂₃BClN₅O₄ [M+H]⁺: 516.2; found 516.2.

### 2-((4S)-6-(4-chlorophenyl)-8-methoxy-1-methyl-4H-benzo[f][1,2,4]triazolo[4,3-a][1,4]diazepin-4-yl)-N-phenylacetamide (BRD-E74c)

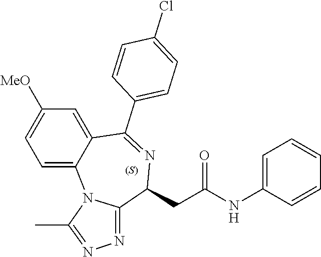

2-((4S)-6-(4-chlorophenyl)-8-methoxy-1-methyl-4H-benzo[f][1,2,4]triazolo[4,3-a][1,4]diazepin-4-yl)-N-phenylacetamide (BRD-E74c) was synthesized by following the method of general EDC coupling of 39 (100 mg, 0.25 mmol) and aniline (30 μL, 0.38 mmol). BRD-E74c (60 mg, 50.4%) was isolated by flash chromatography as a pale yellow solid. ¹H-NMR (400 MHz, DMSO-d₆): δ 10.32 (s, 1H), 7.81 (d, J=8.80 Hz, 1H), 7.63 (d, J=7.6 Hz, 2H), 7.54-7.46 (m, 4H), 7.39 (dd, J=2.8, 8.8 Hz, 1H), 7.31 (t, J=8.4 Hz, 2H), 7.05 (t, J=7.2 Hz, 1H), 6.90 (d, J=2.8 Hz, 1H), 4.58 (q, J=6.0 Hz, 1H), 3.80 (s, 3H), 3.56-3.42 (m, 2H), 2.55 (s, 3H). LRMS m/z: calcd for C₂₆H₂₂ClN₅O₂ [M+H]⁺:472.2; found 472.2.

### 2-((4S)-6-(4-chlorophenyl)-8-methoxy-1-methyl-4H-benzo[f][1,2,4]triazolo[4,3-a][1,4]diazepin-4-yl)-N-(3,4-dihydroxybenzyl)acetamide(BRD-N09)

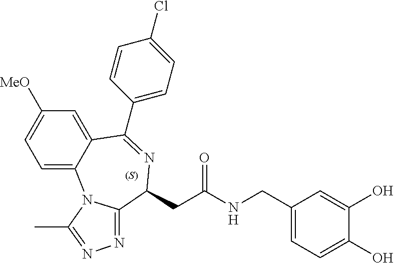

2-((4S)-6-(4-chlorophenyl)-8-methoxy-1-methyl-4H-benzo[f][1,2,4]triazolo[4,3-a][1,4]diazepin-4-yl)-N-(3,4-dihydroxybenzyl)acetamide (BRD-N09) was synthesized by following the method of general HATU coupling of 39 (75 mg, 0.19 mmol) and 3,4-dihydroxybenzylamine (46.6 mg, 0.24 mmol). BRD-N09 (30 mg, 30.6%) was isolated by flash chromatography as a pale yellow solid. ¹H-NMR (400 MHz, CD₃OD): δ 8.73 (t, J=6.0 Hz, 1H), 7.73 (d, J=8.8 Hz, 1H), 7.41-7.37 (m, 5H), 6.92 (d, J=2.8 Hz, 1H), 6.83 (d, J=1.6 Hz, 1H), 6.77-6.70 (m, 2H), 4.64 (q, J=4.4 Hz, 1H), 4.50-4.45 (m, 1H), 4.14 (m, 1H), 3.84 (s, 3H), 3.50-3.44 (m, 1H), 3.21 (dd, J=4.0, 14.4 Hz, 1H), 2.65 (s, 3H). LRMS m/z: calcd for C₂₇H₂₄ClN₅O₄ [M+H]⁺: 518.2; found 518.2.

### (4-((2-((4S)-6-(4-chlorophenyl)-8-methoxy-1-methyl-4H-benzo[f][1,2,4]triazolo[4,3-a][1,4]diazepin-4-yl)acetamido)methyl)phenyl)boronic acid (BRD-E09)

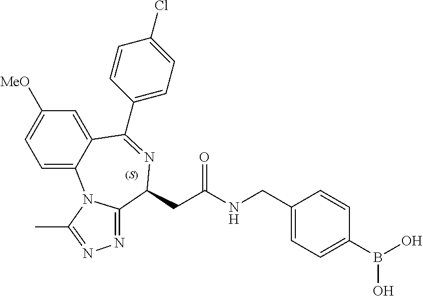

(4-((2-((4S)-6-(4-chlorophenyl)-8-methoxy-1-methyl-4H-benzo[f][1,2,4]triazolo[4,3-a][1,4]diazepin-4-yl)acetamido)methyl)phenyl)boronic acid (BRD-E09) was synthesized by following the method of general EDC coupling of 39 (75 mg, 0.19 mmol) and (4-(aminomethyl)phenyl)boronic acid (36 mg, 0.19 mmol). BRD-E09 (50 mg, 50%) was isolated by flash chromatography as a yellow solid. ¹H-NMR (400 MHz, CD₃OD): δ 8.90 (t, J=6.0 Hz, 11H), 7.77 (d, J=8.0 Hz, 1H), 7.73 (d, J=9.2 Hz, 1H), 7.63 (d, J=8.0 Hz, 2H), 7.41-7.37 (m, 7H), 6.90 (d, J=2.8 Hz, 1H), 4.68-4.60 (m, 2H), 4.36-4.31 (m, 1H), 3.92 (s, 3H), 3.54-3.48 (m, 1H), 3.29-3.24 (m, 1H), 2.65 (s, 3H). LRMS m/z: calcd for C₂₇H₂₅BClN₅O₄ [M+H]⁺: 530.2; found 530.2.

### N-benzyl-2-((4S)-6-(4-chlorophenyl)-8-methoxy-1-methyl-4H-benzo[f][1,2,4]triazolo[4,3-a][1,4]diazepin-4-yl)acetamide (BRD-E09c)

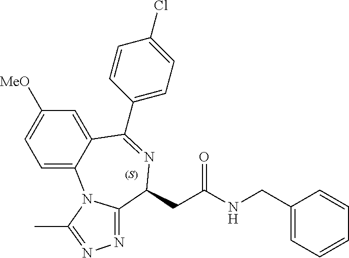

N-benzyl-2-((4S)-6-(4-chlorophenyl)-8-methoxy-1-methyl-4H-benzo[f][1,2,4]triazolo[4,3-a][1,4]diazepin-4-yl)acetamide (BRD-E09c) was synthesized by following the method of general EDC coupling of 39 (75 mg, 0.19 mmol) and benzylamine (30 μL, 0.19 mmol). BRD-E09c (40 mg, 43.5%) was isolated by preparative HPLC as an off-white solid. ¹H-NMR (400 MHz, CD₃OD): δ 7.77 (d, J 9.2 Hz, 1H), 7.51-7.48 (m, 2H), 7.41-7.36 (m, 7H), 7.35-7.30 (m, 1H), 6.95 (d, J=3.2 Hz, 1H), 4.73 (q, J=5.2 Hz, 1H), 4.56 (d, J=14.8 Hz, 1H), 4.38 (d, J=14.8 Hz, 1H), 3.86 (s, 3H), 3.51 (q, J=9.2 Hz, 1H), 3.31-3.29 (m, 1H), 2.74 (s, 3H). LRMS m/z: calcd for C₂₇H₂₄ClN₅O₂ [M+H]⁺: 486.2; found 486.2.

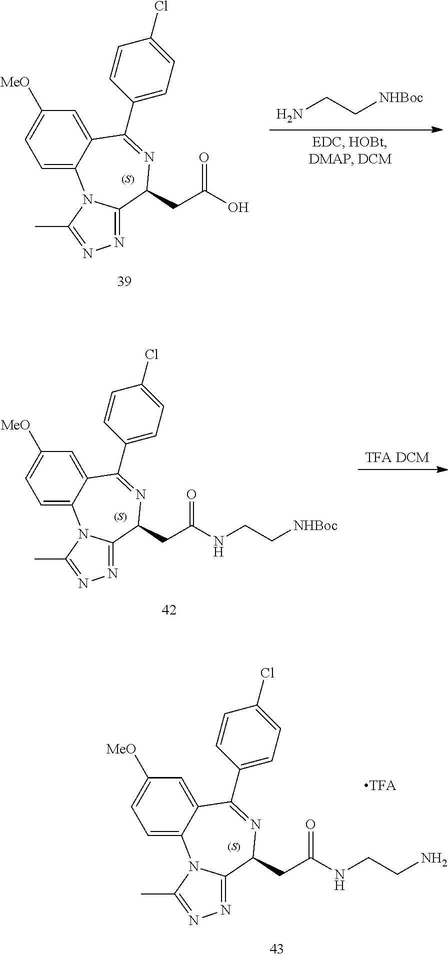

### tert-butyl (2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamate (42)

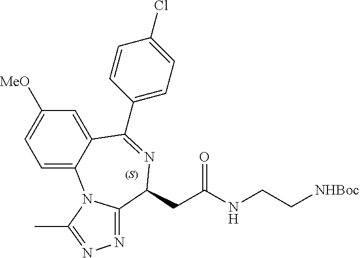

To a stirred solution of 2-((4S)-6-(4-chlorophenyl)-8-methoxy-1-methyl-4H-benzo[f][1,2,4]triazolo[4,3-a][1,4]diazepin-4-yl)acetic acid (39, 600 mg, 1.51 mmol) in dry dichloromethane (10 mL) was added DMAP (277 mg, 2.27 mmol), EDC (435 mg, 2.27 mmol) and HOBt (306 mg, 2.27 mmol). The resulting mixture was stirred at ambient temperature for 15 min at which point the N-Boc-ethylene diamine (363 mg, 2.27 mmol) was added, and the reaction continued to stir for an additional 18 h. At that point, the reaction was diluted with DCM (30 mL) and then washed sequentially with water (10 mL), and brine (10 mL). The organic layer was separated, dried over anhydrous sodium sulphate, filtered, and concentrated under reduced pressure. The reaction was purified by flash chromatography (60-120 mesh, 8-10% MeOH in DCM), and fractions containing the product were concentrated under reduced pressure to afford tert-butyl (2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamate, 42 (600 mg), which was taken on without any further purification.

### N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide (43)

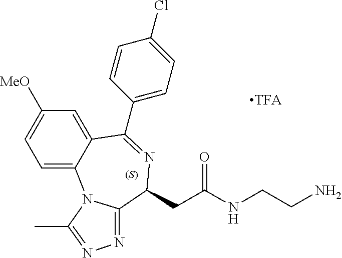

To a stirred, ambient temperature solution of tert-butyl (2-(2-((4S)-6-(4-chlorophenyl)-8-methoxy-1-methyl-4H-benzo[f][1,2,4]triazolo[4,3-a][1,4]diazepin-4-yl)acetamido)ethyl)carbamate, 42 (600 mg, 1.11 mmol) in dichloromethane (20 mL) under nitrogen atmosphere was added trifluoroacetic acid (TFA, 2 mL). The resulting mixture was stirred at ambient temperature for 18 h, at which point it was concentrated under reduced pressure and triturated with diethyl ether (2×10 mL) to obtain N-(2-aminoethyl)-2-((4S)-6-(4-chlorophenyl)-8-methoxy-1-methyl-4H-benzo[f][1,2,4]triazolo[4,3-a][1,4]diazepin-4-yl)acetamide trifluoroacetate salt, 43 (450 mg, 75%) as a yellow solid, which was taken on without any further purification.

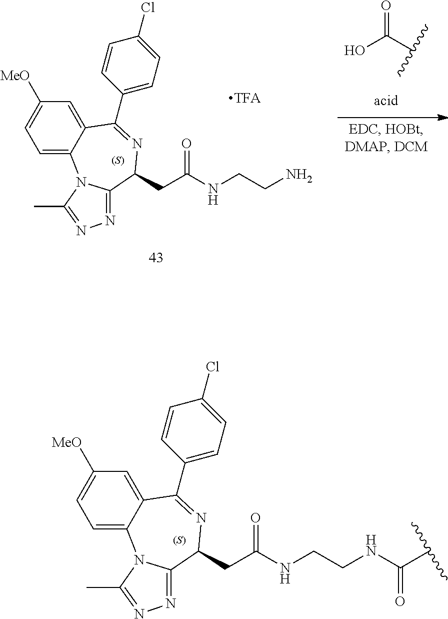

### (4-(2-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-2-oxoethyl)phenyl)boronic acid (BRD-E27)

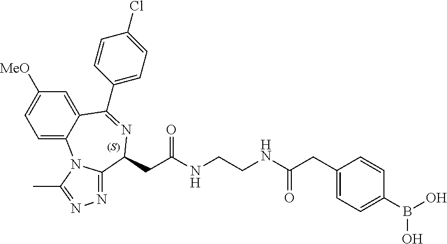

(4-(2-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-2-oxoethyl)phenyl)boronic acid (BRD-E27) was synthesized by following the method of general EDC coupling of 4-carboxymethylphenyl boronic acid (62 mg, 0.34 mmol) and 43 (100 mg, 0.23 mmol). BRD-E27 (30 mg, 22%) was isolated by preparative HPLC as a white solid. ¹H-NMR (400 MHz, DMSO-d₆): δ 8.30 (brs, 1H), 8.06 (brs, 1H) 7.99 (s, 2H), 7.80 (d, J=8.8 Hz, 1H), 7.70 (d, J=8.0 Hz, 1H), 7.54-7.46 (m, 4H), 7.38 (dd, J=2.8 Hz, 8.80 Hz, 1H), 7.21 (d, J=8.0 Hz, 2H), 6.88 (d, J=2.8 Hz, 1H), 4.49 (t, J=8.0 Hz, 1H), 4.01 (m, 1H), 3.79 (s, 3H), 3.41 (s, 1H), 3.22-3.20 (m, 1H), 3.18-3.16 (m, 6H), 2.54 (s, 3H). LRMS m/z: calcd for C₃₀H₃₀BClN₆O₅ [M+H]⁺: 601.2; found 601.2.

### 2-((4S)-6-(4-chlorophenyl)-8-methoxy-1-methyl-4H-benzo[f][1,2,4]triazolo[4,3-a][1,4]diazepin-4-yl)-N-(2-(2-phenylacetamido)ethyl)acetamide (BRD-E27c)

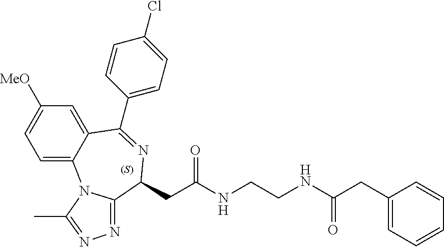

2-((4S)-6-(4-chlorophenyl)-8-methoxy-1-methyl-4H-benzo[1,2,4]triazolo[4,3-a][1,4]diazepin-4-yl)-N-(2-(2-phenylacetamido)ethyl)acetamide (BRD-E27c) was synthesized by following the method of general EDC coupling of phenylacetic acid (46 mg, 0.34 mmol) and 43 (100 mg, 0.23 mmol). BRD-E27c (20 mg, 15.8%) was isolated by preparative HPLC as a white solid. ¹H-NMR (400 MHz, CD₃OD): δ 7.73 (d, J=9.2 Hz, 1H), 7.57-7.54 (m, 2H), 7.44-7.39 (m, 3H), 7.28 (d, J=4.4 Hz, 4H), 7.22-7.20 (m, 1H), 6.94 (d, J=2.8 Hz, 1H), 4.62 (q, J=6.0 Hz, 1H), 3.84 (s, 3H), 3.52 (s, 2H), 3.39-3.35 (m, 6H), 2.65 (s, 3H). LRMS m/z: calcd for C₃₀H₂₉ClN₆O₃ [M+H]⁺: 557.2; found 557.2.

### (4-((E)-3-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-3-oxoprop-1-en-1-yl)phenyl)boronic acid (BRD-E29)

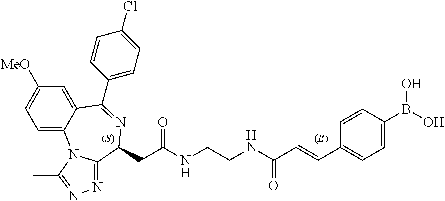

(4-((E)-3-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-3-oxoprop-1-en-1-yl)phenyl)boronic acid (BRD-E29) was synthesized by following the method of general EDC coupling of (E)-4-(2-carboxyvinyl)phenyl boronic acid (66 mg, 0.34 mmol) and 43 (100 mg, 0.23 mmol). BRD-E29 (20 mg, 14.3%) was isolated by preparative HPLC as a white solid. ¹H-NMR (400 MHz, CD₃OD): δ 8.14 (s, 1H), 7.71-7.65 (m, 3H), 7.56-7.51 (m, 5H), 7.42-7.36 (m, 3H), 6.89 (d, J=2.8 Hz, 1H), 6.65 (d, J=16.0 Hz, 1H), 4.65 (q, J=5.6 Hz, 1H), 3.81 (s, 3H), 3.52-3.41 (m, 5H), 3.35 (d, J=5.6 Hz, 2H), 2.64 (s, 3H). LRMS m/z: calcd for C₃₁H₃₀BClN₆O₅ [M+H]⁺: 613.2; found 613.2.

### N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)cinnamamide (BRD-E29c)

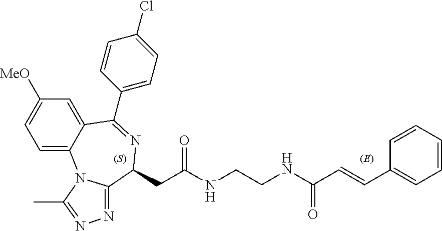

N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)cinnamamide (BRD-E29c) was synthesized by following the method of general EDC coupling of trans-cinnamic acid (51 mg, 0.34 mmol) and 43 (100 mg, 0.23 mmol). BRD-E29c (20 mg, 15.4%) was isolated by preparative HPLC as a white solid. ¹H-NMR (400 MHz, DMSO-d₆): δ 6.89 (d, J=8.8 Hz, 1H), 6.75-6.70 (m, 5H), 6.60-6.56 (m, 6H), 6.09 (d, J=2.8 Hz, 1H), 5.81 (d, J=16.0 Hz, 1H), 3.84 (q, J=5.6 Hz, 1H), 3.00 (s, 3H), 2.69-2.63 (m, 4H), 2.54 (m, 2H), 1.83 (s, 3H). LRMS m/z: calcd for C₃₁H₂₉ClN₆O₃ [M+H]⁺: 569.2; found 569.3.

### (3-((E)-3-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-3-oxoprop-1-en-1-yl)phenyl)boronic acid (BRD-E30)

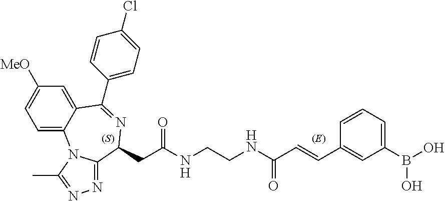

(3-((E)-3-((2-(2-((4S)-6-(4-chlorophenyl)-8-methoxy-1-methyl-4H-benzo[f][1,2,4]triazolo[4,3-a][1,4]diazepin-4-yl)acetamido)ethyl)amino)-3-oxoprop-1-en-1-yl)phenyl)boronic acid (BRD-E30) was synthesized by following the method of general EDC coupling of (E)-3-(2-carboxyvinyl)phenyl boronic acid (66 mg, 0.34 mmol) and 43 (100 mg, 0.23 mmol). BRD-E30 (35 mg, 25%) was isolated by preparative HPLC as an off-white solid. ¹H-NMR (400 MHz, CD₃OD): δ 7.78 (s, 1H), 7.70 (d, J=8.8 Hz, 1H), 7.64 (d, J=7.6 Hz, 1H), 7.60-7.53 (m, 4H), 7.43-7.36 (m, 4H), 6.89 (d, J=2.8 Hz, 1H), 6.63 (d, J=15.6 Hz, 1H), 4.65 (q, J=5.6 Hz, 1H), 3.81 (s, 3H), 3.52-3.44 (m, 5H), 3.36 (d, J=2.8 Hz, 3H), 2.64 (s, 3H). LRMS m/z: calcd for C₃₁H₃₀BClN₆O₅ [M+H]⁺: 613.2; found 613.2.

### (E)-N-(2-(2-((4S)-6-(4-chlorophenyl)-8-methoxy-1-methyl-4H-benzo[f][1,2,4]triazolo[4,3-a][1,4]diazepin-4-yl)acetamido)ethyl)-3-(3-hydroxyphenyl)acrylamide (BRD-E30c)

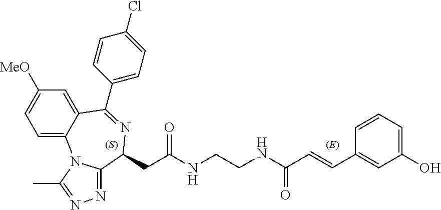

(E)-N-(2-(2-((4S)-6-(4-chlorophenyl)-8-methoxy-1-methyl-4H-benzo[1,2,4]triazolo[4,3-a][1,4]diazepin-4-yl)acetamido)ethyl)-3-(3-hydroxyphenyl)acrylamide (BRD-E30c) was synthesized by following the method of general EDC coupling of trans-3-hydroxycinnamic acid (56 mg, 0.34 mmol) and 43 (100 mg, 0.23 mmol). BRD-E30c (10 mg, 7.5%) was isolated by preparative HPLC as an off-white solid. ¹H-NMR (400 MHz, CD₃OD): δ 8.18 (s, 1H), 7.70 (d, J=8.8 Hz, 1H), 7.56-7.53 (m, 2H), 7.46-7.36 (m, 4H), 7.22 (t, J=7.6 Hz, 1H), 7.01 (d, J=7.6 Hz, 1H), 6.96 (t, J=2.0 Hz, 1H), 6.90 (d, J=2.8 Hz, 1H), 6.83-6.81 (m, 1H), 6.54 (d, J=15.6 Hz, 1H), 4.65 (q, J=6.0 Hz, 1H), 3.82 (s, 3H), 3.50-3.40 (m, 4H), 3.34 (m, 2H), 2.64 (s, 3H). LRMS m/z: calcd for C₃₁H₂₉ClN₆O₃ [M+H]⁺: 585.2; found 585.2.

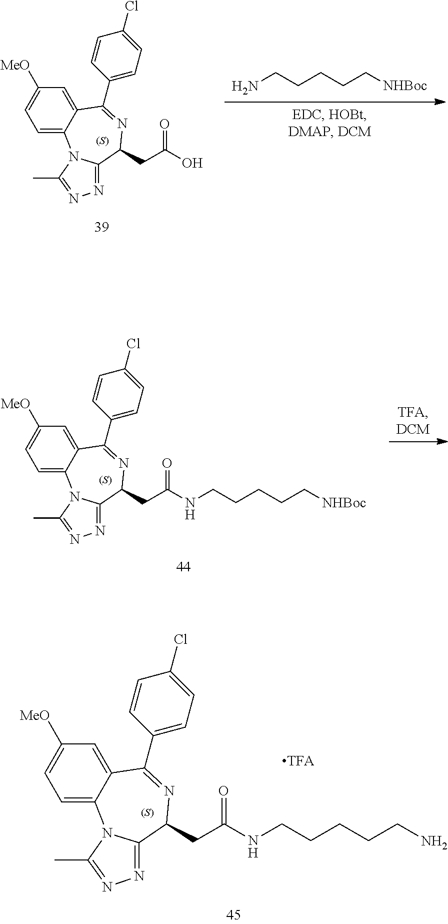

### tert-butyl (5-(2-((4S)-6-(4-chlorophenyl)-8-methoxy-1-methyl-4H-benzo[f][1,2,4]triazolo[4,3-a][1,4]diazepin-4-yl)acetamido)pentyl)carbamate (44)

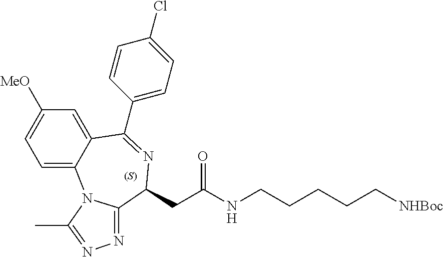

To a stirred solution of 2-((4S)-6-(4-chlorophenyl)-8-methoxy-1-methyl-4H-benzo[f][1,2,4]triazolo[4,3-a][1,4]diazepin-4-yl)acetic acid (39, 300 mg, 0.76 mmol) in dry dichloromethane (10 mL) was added DMAP (139 mg, 1.13 mmol), EDC (217 mg, 1.13 mmol) and HOBt (153 mg, 1.13 mmol). The resulting mixture was stirred at ambient temperature for 15 min at which point the N-Boc-1,5-diaminopentane (230 mg, 1.134 mmol) was added, and the reaction continued to stir for an additional 18 h. At that point, the reaction was diluted with DCM (30 mL) and then washed sequentially with water (10 mL), and brine (10 mL). The organic layer was separated, dried over anhydrous sodium sulphate, filtered, and concentrated under reduced pressure. The reaction was purified by flash chromatography (60-120 mesh, 8-10% MeOH in DCM), and fractions containing the product were concentrated under reduced pressure to afford tert-butyl (5-(2-((4S)-6-(4-chlorophenyl)-8-methoxy-1-methyl-4H-benzo[f][1,2,4]triazolo[4,3-a][1,4]diazepin-4-yl)acetamido)pentyl)carbamate, 44 (320 mg, 72.9%), which was taken on without any further purification.

### N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide trifluoroacetate salt (45)

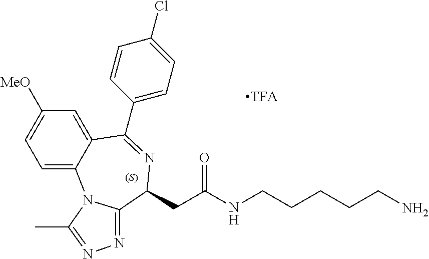

To a stirred, ambient temperature solution of tert-butyl (5-(2-((4S)-6-(4-chlorophenyl)-8-methoxy-1-methyl-4H-benzo[f][1,2,4]triazolo[4,3-a][1,4]diazepin-4-yl)acetamido)pentyl)carbamate, 44 (320 mg, 0.55 mmol) in dichloromethane (10 mL) under nitrogen atmosphere was added trifluoroacetic acid (TFA, 2 mL). The resulting mixture was stirred at ambient temperature for 18 h, at which point it was concentrated under reduced pressure and triturated with diethyl ether (2×10 mL) to obtain N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide trifluoroacetate salt, 45 (250 mg, 94%) as a yellow solid, which was taken on without any further purification.

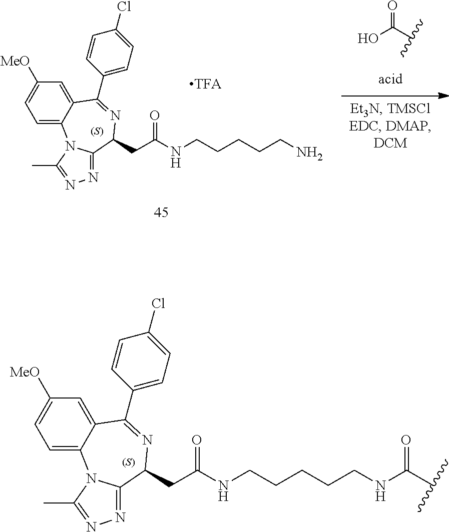

### N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)-2,3-dihydroxybenzamide (BRD-N08)

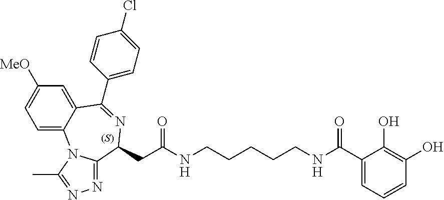

To a stirred, 0° C. solution of 2,3-dihydroxybenzoic acid (170 mg, 1.09 mmol) in dry dichloromethane (4 mL) was added triethylamine (0.4 mL, 3.11 mmol) and then trimethylsilyl chloride (0.3 mL, 2.80 mmol) dropwise. The resulting solution was warmed to ambient temperature and stirred for 3 h, at which point EDC (90 mg, 0.47 mmol) and DMAP (58 mg, 0.47 mmol) were added. The mixture was stirred at ambient temperature for 15 min, at which point N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide trifluoroacetate salt, 45 (150 mg, 0.31 mmol) was added. The resulting mixture was stirred at ambient temperature for 18 h, at which point it was diluted with dichloromethane (10 mL), then washed with water (5 mL) and brine (5 ml). The organic layer was separated, dried over anhydrous sodium sulphate, filtered, and concentrated under reduced pressure, then purified by preparatory HPLC [column: X-Select C18 (19×150 mm, 5 m); mobile phase A: 0.1% formic acid in water; mobile phase B: ACN; flowrate: 15 mL/min]. Fractions containing the product were lyophilized to afford N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)-2,3-dihydroxybenzamide, BRD-N08 (14 mg, 7.3%) as a white solid. ¹H-NMR (400 MHz, CD₃OD): δ 7.73 (d, J=8.8 Hz, 1H), 7.56 (d, J=4.8 Hz, 2H), 7.45-7.37 (m, 3H), 7.23 (d, J=6.8 Hz, 1H), 6.94-6.90 (m, 2H), 6.69 (t, J=8.0 Hz, 1H), 4.64 (q, J=5.2 Hz, 1H), 3.84 (s, 3H), 3.38 (m, 3H), 3.29 (m, 2H), 2.65 (s, 3H), 1.66 (m, 4H), 1.47 (m, 2H). LRMS m/z: calcd for C₃₂H₃₃ClN₆O₅ [M+H]⁺: 617.2; found 617.2.

### N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)benzamide (BRD-N08c)

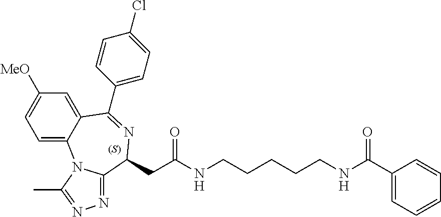

N-(5-(2-((4S)-6-(4-chlorophenyl)-8-methoxy-1-methyl-4H-benzo[f][1,2,4]triazolo[4,3-a][1,4]diazepin-4-yl)acetamido)pentyl)benzamide (BRD-N08c) was synthesized by following the method of general EDC coupling of benzoic acid (35 mg, 0.31 mmol) and 45 (100 mg, 0.23 mmol). BRD-N08c (40 mg, 32.9%) was isolated by preparative HPLC as a white solid. ¹H-NMR (400 MHz, CD₃OD): δ 8.46-8.38 (m, 1H), 8.10 (s, 1H), 7.83-7.81 (m, 2H), 7.74 (d, J=8.8 Hz, 1H), 7.56-7.50 (m, 3H), 7.50-7.39 (m, 5H), 6.93 (d, J=2.8 Hz, 1H), 4.64 (q, J=5.2 Hz, 1H), 3.84 (s, 3H), 3.45-3.39 (m, 3H), 3.32-3.23 (m, 3H), 2.65 (s, 3H), 1.71-1.64 (m, 4H), 1.53-1.47 (m, 2H). LRMS m/z: calcd for C₃₂H₃₃ClN₆O₃ [M+H]⁺: 585.2; found 585.4.

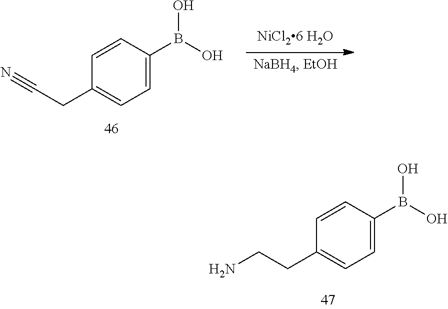

### (4-(2-aminoethyl)phenyl)boronic acid (47)

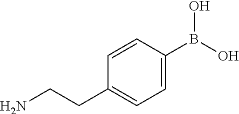 [SMILES: NCCc1ccc(B(O)O)cc1]

To a stirred solution of (4-(cyanomethyl)phenyl)boronic acid, 46 (500 mg. 3.11 mmol) in ethanol (20 mL) at ambient temperature was added nickel(II) chloride hexahydrate (400 mg, 3.11 mmol), followed by sodium borohydride (350 mg, 9.32 mmol). The resulting mixture was stirred for 18 h, then filtered through a pad of Celite. The Celite was washed with ethanol (3×20 mL) and the combined filtrates were concentrated under reduced pressure; the resulting residue was diluted with water (10 mL) and extracted with ethyl acetate (3×30 mL). The combined organic layers were washed with brine, dried over anhydrous sodium sulphate, filtered, and concentrated under reduced pressure. The resulting crude product, 47 (400 mg) was used without further purification.

### (4-(2-(2-((4S)-6-(4-chlorophenyl)-8-methoxy-1-methyl-4H-benzo[f][1,2,4]triazolo[4,3-a][1,4]diazepin-4-yl)acetamido)ethyl)phenyl)boronic acid (BRD-E14)

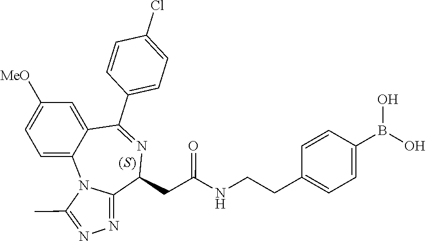

(4-(2-(2-((4S)-6-(4-chlorophenyl)-8-methoxy-1-methyl-4H-benzo[f][1,2,4]triazolo[4,3-a][1,4]diazepin-4-yl)acetamido)ethyl)phenyl)boronic acid (BRD-E14) was synthesized by following the method of general HATU coupling of 39 (200 mg, 0.50 mmol) and (4-(2-aminoethyl)phenyl)boronic acid, 47 (170 mg, 1.01 mmol). BRD-E14 (40 mg, 14.6%) was isolated by preparative HPLC as a pale yellow solid. ¹H-NMR (400 MHz, CD₃OD): δ 8.42 (brs, 1H), 7.72 (m, 2H), 7.57-7.50 (m, 3H), 7.43-7.38 (m, 3H), 7.28-7.21 (m, 2H), 6.94 (d, J=2.8 Hz, 1H), 4.62 (q, J=5.2 Hz, 1H), 3.85 (s, 3H), 3.55-3.50 (m, 2H), 3.40 (m, 2H), 3.28-3.22 (m, 1H), 2.88 (t, J=7.2 Hz, 2H), 2.66 (s, 3H). LRMS m/z: calcd for C₂₈H₂₇BClN₅O₄ [M+H]⁺: 544.2; found 544.2.

### 2-((4S)-6-(4-chlorophenyl)-8-methoxy-1-methyl-4H-benzo[f][1,2,4]triazolo[4,3-a][1,4]diazepin-4-yl)-N-phenethylacetamide (BRD-E14c)

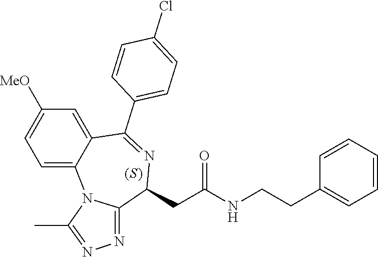

2-((4S)-6-(4-chlorophenyl)-8-methoxy-1-methyl-4H-benzo[f][1,2,4]triazolo[4,3-a][1,4]diazepin-4-yl)-N-phenethylacetamide (BRD-E14c) was synthesized by following the method of general HATU coupling of 39 (75 mg, 0.19 mmol) and phenethylamine (30 μL, 0.38 mmol). BRD-E14c (60 mg, 63.5%) was isolated by preparative HPLC as a pale yellow solid. ¹H-NMR (400 MHz, CD₃OD): δ 8.41 (brs, 1H), 7.73 (d, J=9.2 Hz, 1H), 7.56-7.53 (m, 2H), 7.44-7.39 (m, 3H), 7.30-7.24 (m, 4H), 7.22-7.18 (m, 1H), 6.94 (d, J=2.8 Hz, 1H), 4.63 (q, J=5.2 Hz, 1H), 3.85 (s, 3H), 3.53-3.48 (m, 2H), 3.43-3.36 (m, 1H), 3.29-3.24 (m, 1H), 2.86 (t, J=7.2 Hz, 2H), 2.65 (s, 3H). LRMS m/z: calcd for C₂₈H₂₆ClN₅O₂ [M+H]⁺: 500.2; found 500.2.

### (S)-2-(6-(4-chlorophenyl)-8-methoxy-1-methyl-4H-benzo[f][1,2,4]triazolo[4,3-a][1,4]diazepin-4-yl)-N-(3,4-dihydroxyphenethyl)acetamide (BRD-N10)

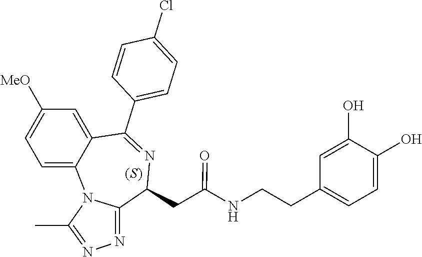

(S)-2-(6-(4-chlorophenyl)-8-methoxy-1-methyl-4H-benzo[f][1,2,4]triazolo[4,3-a][1,4]diazepin-4-yl)-N-(3,4-dihydroxyphenethyl)acetamide (BRD-N10) was synthesized by following the method of general HATU coupling of 39 (100 mg, 0.25 mmol) and 4-(2-aminoethyl)benzene-1,2-diol (53 mg, 0.28 mmol). BRD-N10 (15 mg, 11.2%) was isolated by preparative HPLC as an off-white solid. ¹H-NMR (400 MHz, CD₃OD): δ 7.74 (d, J=8.8 Hz, 1H), 7.54-7.51 (m, 2H), 7.45-7.39 (m, 3H), 6.94 (d, J=2.8 Hz, 1H), 6.69-6.67 (m, 2H), 6.58-6.55 (m, 1H), 4.63 (q, J=5.2 Hz, 1H), 3.85 (s, 3H), 3.46-3.46 (m, 2H), 3.33 (m, 1H), 3.29-3.24 (m, 2H), 2.71 (t, J=7.2 Hz, 1H), 2.66 (s, 3H). LRMS m/z: calcd for C₂₈H₂₇BClN₅O₄ [M+H]⁺: 532.2; found 532.2.

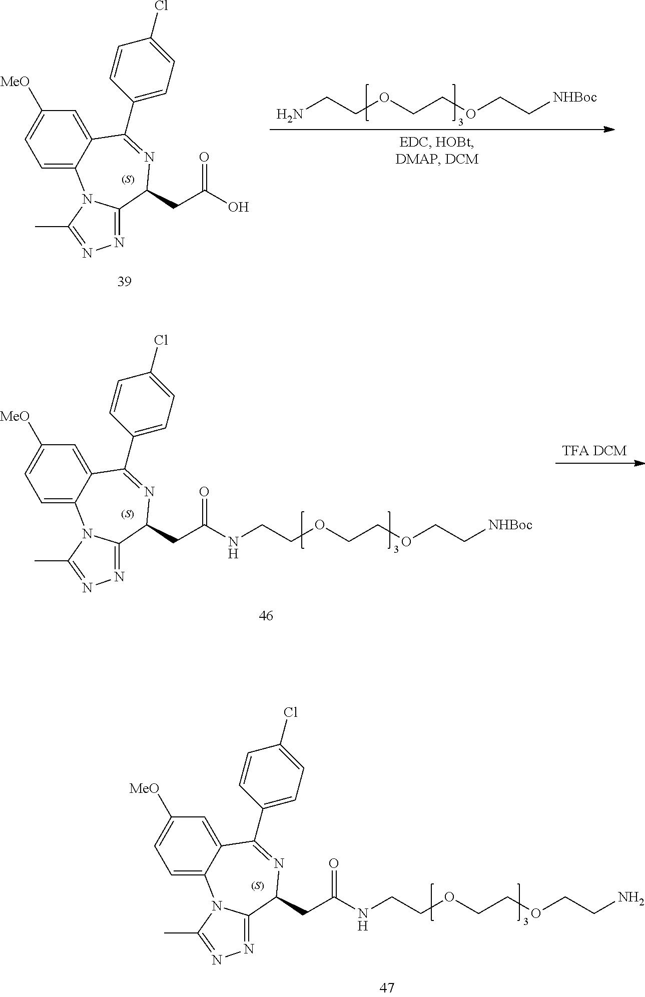

### tert-butyl (1-((4S) -6(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)carbamate (46)

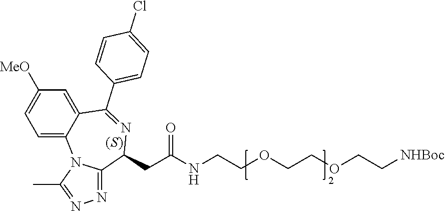

tert-butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)carbamate, 46 (800 mg, 49%) was synthesized by following the method of general EDC coupling of 39 (707) mg, 1.78 mmol) and tert-butyl (14-amino-3,6,9,12-tetraoxatetradecyl)carbamate (500 mg, 1.49 mmol). tert-butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)carbamate, 46 (800 mg, 49%) was isolated by flash chromatography.

### N-(14-amino-3,6,9,12-tetraoxatetradecyl)-2-((4S)-6-(4-chlorophenyl)-8-methoxy-1-methyl-4H-benzo[f][1,2,4]triazolo[4,3-a][1,4]diazepin-4-yl)acetamide (47)

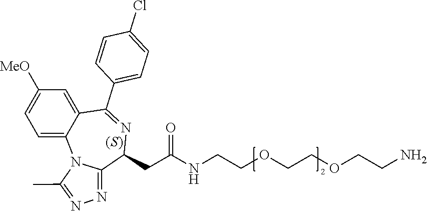

To a stirred, ambient temperature solution of tert-butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)carbamate, 46 (800 mg, 1.12 mmol) in dichloromethane (20 mL) under nitrogen atmosphere was added trifluoroacetic acid (2 mL). The resulting mixture was stirred at ambient temperature for 18 h, at which point it was concentrated under reduced pressure and triturated with diethyl ether (2×10 mL) to obtain N-(14-amino-3,6,9,12-tetraoxatetradecyl)-2-((4S)-6-(4-chlorophenyl)-8-methoxy-1-methyl-4H-benzo[f][1,2,4]triazolo[4,3-a][1,4]diazepin-4-yl)acetamide, 47 (350 mg, 40.7%) as a yellow solid, which was taken on without any further purification.

### N-(1-((4S)-6-(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)-3-hydroxybenzamide (BRD-N69c)

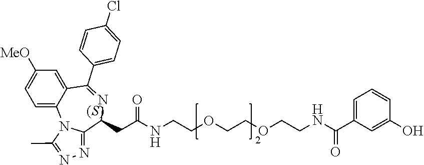

N-(1-((4S)-6-(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)-3-hydroxybenzamide (BRD-N69c) was synthesized by following the method of general EDC coupling of 3-hydroxybenzoic acid (170 mg, 0.32 mmol) and N-(14-amino-3,6,9,12-tetraoxatetradecyl)-2-((4S)-6-(4-chlorophenyl)-8-methoxy-1-methyl-4H-benzo[f][1,2,4]triazolo[4,3-a][1,4]diazepin-4-yl)acetamide (100 mg, 0.32 mmol). N-(1-((4S)-6-(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)-3-hydroxybenzamide, BRD-N69c (13 mg, 10.9%) was isolated by preparative HPLC as a white solid. ¹H-NMR (400 MHz, CD₃OD): δ 8.08 (s, 1H), 7.73 (d, J=8.8 Hz, 1H), 7.58-7.55 (m, 2H), 7.45-7.38 (m, 3H), 7.26-7.23 (m, 3H), 6.95-6.92 (m, 2H), 4.65 (q, J=5.2 Hz, 1H), 3.84 (s, 3H), 3.66-3.59 (m, 17H), 3.56-3.50 (m, 2H), 3.46-3.42 (m, 3H), 2.66 (s, 3H). LRMS m/z: calcd for C₃₇H₄₃ClN₆O₈ [M+H]⁺: 735.3; found 735.2.

### Phenol Scaffold Compounds—Synthesis of BRD-N25C, BRD-E21 and BRD E21C

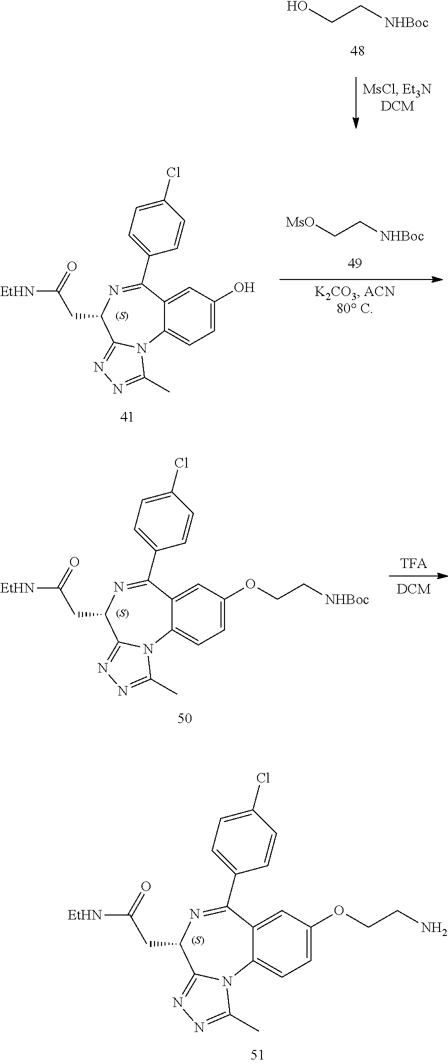

The phenol scaffold compounds (BRD-N25c, BRD-E21, and BRD-E21c) were synthesized using processes disclosed in WO2013033270, to Arnold et al., which is hereby incorporated by reference in its entirety.

### 2-((tert-butoxycarbonyl)amino)ethyl methanesulfonate (49)

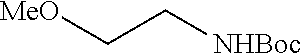 [SMILES: COCCNC(=O)OC(C)(C)C]

To a stirred, 0° C. solution of tert-butyl (2-hydroxyethyl)carbamate, 48 (1 g, 6.20 mmol) in dry dichloromethane (5 mL), was added triethylamine (1.1 mL, 12.41 mmol) and mesyl chloride (0.95 mL, 12.41 mmol) under a nitrogen atmosphere. The reaction mixture was warmed to ambient temperature and stirred for 5 hours, at which point it was diluted with dichloromethane (20 mL), then washed with water (10 mL) and brine (10 ml). The organic layer was dried over anhydrous sodium sulphate, filtered, and concentrated under reduced pressure to afford 2-((tert-butoxycarbonyl)amino)ethyl methanesulfonate, 49 (1.4 g, 94.6%), which was taken on without further purification.

### tert-butyl (2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate (50)

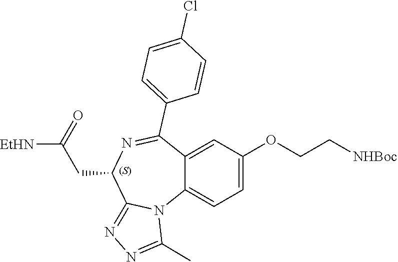

To a stirred solution of 2-((4S)-6-(4-chlorophenyl)-8-hydroxy-1-methyl-4H-benzo[f][1,2,4]triazolo[4,3-a][1,4]diazepin-4-yl)-N-ethylacetamide, 41 (580 mg, 1.42 mmol) in dry DMF (10 mL) and acetonitrile (10 mL) was added potassium carbonate (391 mg, 2.83 mmol) and 2-((tert-butoxycarbonyl)amino)ethyl methanesulfonate, 49 (508 mg. 2.12 mmol). The resulting mixture was heated at 90° C. for 6 h, at which point it was diluted with ethyl acetate (30 mL), then washed with ice water (10 mL) and brine (10 mL). The organic layer was separated, dried over anhydrous sodium sulphate, filtered, and concentrated under reduced pressure then purified by flash chromatography (60-120 mesh, 8-10% MeOH in DCM). Fractions containing the desired product were concentrated under reduced pressure to afford tert-butyl (2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate, 50 (250 mg, 31.9%).

### 2-((4S)-8-(2-aminoethoxy)-6-(4-chlorophenyl)-1-methyl-4H-benzo[f][1,2,4]triazolo[4,3-a][1,4]diazepin-4-yl)-N-ethylacetamide (51)

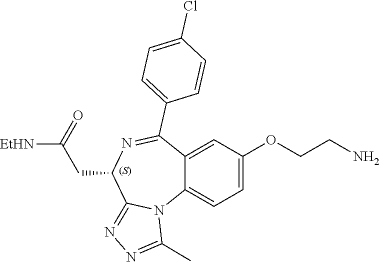

To a stirred, ambient temperature solution of tert-butyl (2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamate, 50 (250 mg, 1.12 mmol) in dichloromethane (20 mL) under nitrogen atmosphere was added trifluoroacetic acid (2 mL). The resulting mixture was stirred at ambient temperature for 18 h, at which point it was concentrated under reduced pressure and triturated with diethyl ether (2×10 mL) to obtain 2-((4S)-8-(2-aminoethoxy)-6-(4-chlorophenyl)-1-methyl-4H-benzo[f][1,2,4]triazolo[4,3-a][1,4]diazepin-4-yl)-N-ethylacetamide, 51 (200 mg, 40.7%) as a brown gummy solid, which was taken on without any further purification.

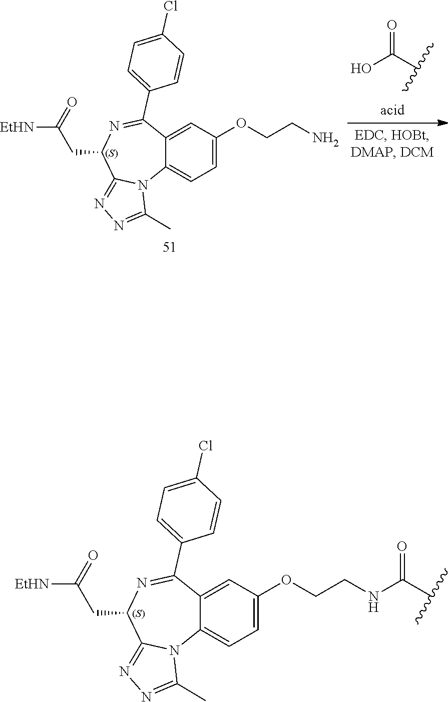

### N-(2-(((4S)-6-(4-chlorophenyl)-4-(2-(ethylamino)-2-oxoethyl)-1-methyl-4H-benzo[f][1,2,4]triazolo[43-a][1,4]diazepin-8-yl)oxy)ethyl)-3-hydroxybenzamide (BRD-N25c)

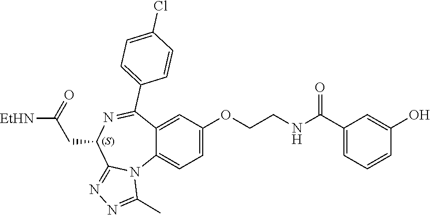

N-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)-3-hydroxybenzamide (BRD-N25c) was synthesized by following the method of general EDC coupling of 3-hydroxybenzoic acid (34 mg, 0.25 mmol) and 2-((4S)-8-(2-aminoethoxy)-6-(4-chlorophenyl)-1-methyl-4H-benzo[f][1,2,4]triazolo[4,3-a][1,4]diazepin-4-yl)-N-ethylacetamide, 51 (75 mg, 0.16 mmol). N-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)-3-hydroxybenzamide, BRD-N25c (9.2 mg, 9.8%) was isolated by preparative HPLC. ¹H-NMR (400 MHz, CD₃OD): δ 7.72 (d, J=8.8 Hz, 1H), 7.54 (d, J=8.8 Hz, 2H), 7.46-7.40 (m, 3H), 7.29-7.21 (m, 3H), 6.99-6.94 (m, 2H), 4.63 (q, J=5.6 Hz, 1H), 4.24-4.20 (m, 2H), 3.75 (t, J=5.6 Hz, 2H), 3.41-3.37 (m, 1H), 3.27-3.22 (m, 3H), 2.64 (s, 3H), 1.20 (t, J=7.2 Hz, 3H). LRMS m/z: calcd for C₃₀H₂₉ClN₉O₄ [M+H]⁺: 573.2; found 573.2.

### (3-((2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamoyl)phenyl)boronic acid (BRD-E21)

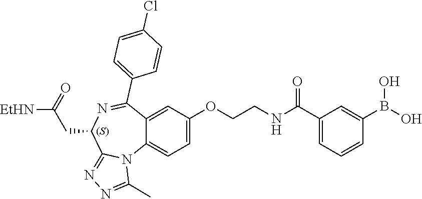

(3-((2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamoyl)phenyl)boronic acid (BRD-E21) was synthesized by following the method of general EDC coupling of 3-boronobenzoic acid (41 mg, 0.25 mmol) and 2-((4S)-8-(2-aminoethoxy)-6-(4-chlorophenyl)-1-methyl-4H-benzo[f][1,2,4]triazolo[4,3-a][1,4]diazepin-4-yl)-N-ethylacetamide, 51 (75 mg, 0.16 mmol). (3-((2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)carbamoyl)phenyl)boronic acid, BRD-E2 1 (13.6 mg, 13.3%) was isolated by preparative HPLC. ¹H-NMR (400 MHz, CD₃OD): δ 8.29 (brs, 1H), 8.06 (s, 1H), 7.82-7.79 (m, 2H), 7.73 (d, J=8.8 Hz, 1H), 7.58-7.53 (m, 2H), 7.46-7.39 (m, 4H), 6.99 (d, J=2.8 Hz, 1H), 4.62 (q, J=5.2 Hz, 1H), 4.27-4.20 (m, 2H), 3.78 (t, J=5.6 Hz, 2H), 3.43-3.35 (m, 1H), 3.33-3.22 (m, 3H), 2.63 (s, 3H), 1.20 (t, J=7.2 Hz, 3H). LRMS m/z: calcd for C₃₀H₃₀BClN₆O₅ [M+H]⁺: 601.2; found 601.2.

### N-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)benzamide (BRD-E2 1c)

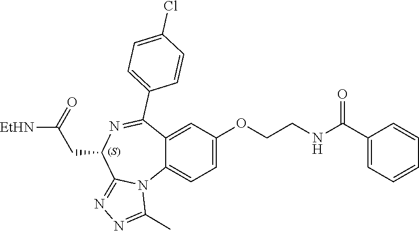

N-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)benzamide (BRD-E21c) was synthesized by following the method of general EDC coupling of benzoic acid (30 mg, 0.25 mmol) and 2-((4S)-8-(2-aminoethoxy)-6-(4-chlorophenyl)-1-methyl-4H-benzo[f][1,2,4]triazolo[4,3-a][1,4]diazepin-4-yl)-N-ethylacetamide, 51 (75 mg, 0.16 mmol). N-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethyl)benzamide, BRD-E21c (19.0 mg, 21.7%) was isolated by preparative HPLC. ¹H-NMR (400 MHz, CD₃OD): δ 7.83-7.80 (m, 2H), 7.73 (d, J=9.2 Hz, 1H), 7.55-7.53 (m, 3H), 7.49-7.39 (m, 5H), 7.00 (d, J=3.2 Hz, 1H), 4.63 (q, J=5.2 Hz, 2H), 4.26-4.19 (m, 2H), 3.78 (t, J=5.6 Hz, 2H), 3.43-3.37 (m, 1H), 3.33-3.15 (m, 2H), 2.64 (s, 3H), 1.20 (t, J=7.2 Hz, 3H). LRMS m/z: calcd for C₃₀H₂₉ClN₆O₃ [M+H]⁺: 557.2; found 557.2.

### SYNTHESIS OF BRD-N22, BRD-N22c, BRD-E20 AND BRD-E20C

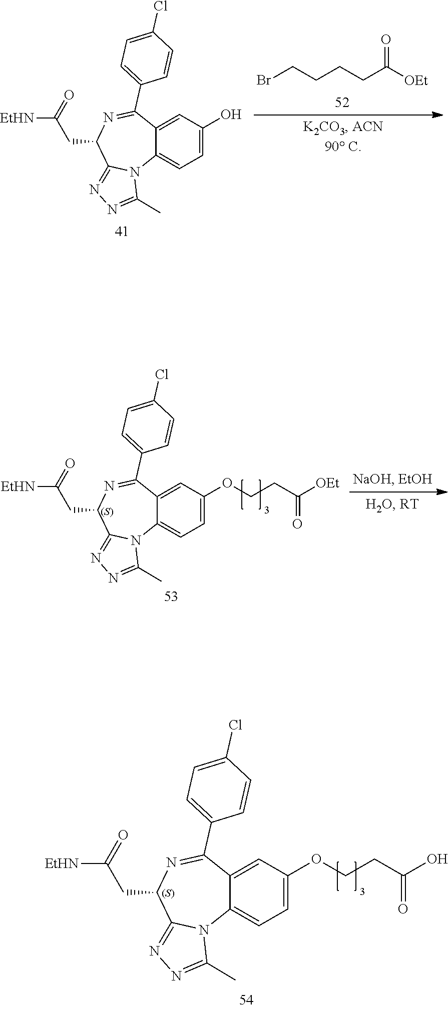

BRD-N22, BRD-N22c, BRD-E20 and BRD-E20c were synthesized using processes disclosed in WO2013033270, and WO2015081280 to Arnold et al., which is hereby incorporated by reference in its entirety.

### ethyl 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoate (53)

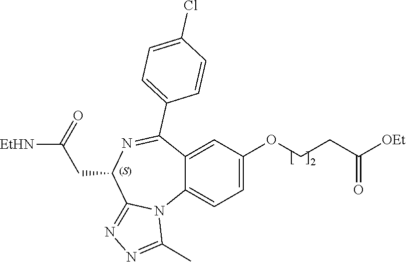

To a stirred solution of 2-((4S)-6-(4-chlorophenyl)-8-hydroxy-1-methyl-4H-benzo[f][1,2,4]triazolo[4,3-a][1,4]diazepin-4-yl)-N-ethylacetamide, 41 (500 mg, 1.22 mmol) in dry acetonitrile (10 mL) at ambient temperature was added potassium carbonate (505 mg, 3.66 mmol) and ethyl 5-bromopentanoate (281 mg, 1.34 mmol) under a nitrogen atmosphere. The resulting solution was then heated to 90° C. for 12 h, at which point it was diluted with ethyl acetate (30 mL), then washed with ice water (10 mL) and brine (10 mL). The organic layer was separated, dried over anhydrous sodium sulphate, filtered, and concentrated under reduced pressure, then purified by flash chromatography (60-120 mesh, 8-10% MeOH in DCM). Fractions containing the desired product were combined and concentrated under reduced pressure to afford ethyl 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoate, 53 (500 mg, 76%), which was taken on without further purification.

### 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid (54)

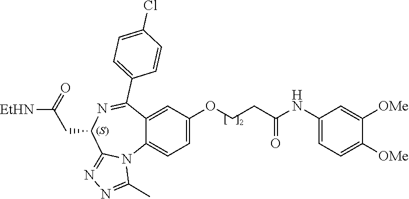

To a stirred solution of ethyl 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoate, 53 (500 mg, 0.98 mmol) in ethanol (10 mL) and water (10 mL) was added sodium hydroxide (186 mg, 4.90 mmol). The resulting solution was stirred at ambient temperature for 3 h before being acidified to pH 3 with 1.5N HCl and extracted with dichloromethane (20 mL). The organic layer was separated, dried over anhydrous sodium sulphate, filtered, and concentrated under reduced pressure. The remaining residue was triturated with diethyl ether (10 mL) to obtain 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid, 54 (400 mg, 80%), which was taken on without further purification.

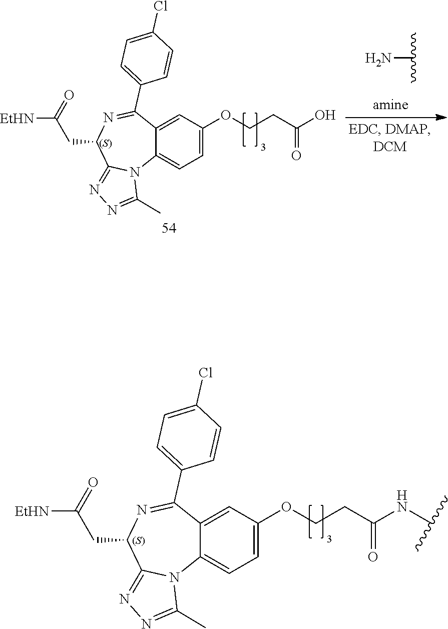

### 5-(((4S)-6-(4-chlorophenyl)-4-(2-(ethylamino)-2-oxoethyl)-1-methyl-4H-benzo[f][1,2,4]triazolo[4,3-a][1, 4]diazepin-8-yl)oxy)-N-(3,4-dimethoxyphenyl)pentanamide (BRD-N22d)

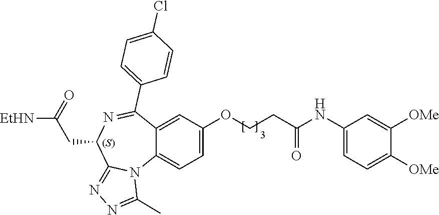

5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dimethoxyphenyl)pentanamide (BRD-E22d) was synthesized by following the method of general EDC coupling of 3,4-dimethoxyaniline (45 mg, 0.29 mmol) and 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid, 54 (100 mg, 0.2 mmol). 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dimethoxyphenyl)pentanamide, BRD-E22d (100 mg, 79%) was isolated by flash chromatography. ¹H-NMR (400 MHz, CD₃OD): δ 7.71 (d, J=8.8 Hz), 7.56-7.53 (m, 2H), 7.43-7.37 (m, 3H), 7.33-7.30 (m, 1H), 7.05-7.96 (m, 1H), 6.93-6.88 (m, 2H), 4.63 (q, J=5.2 Hz, 1H), 4.07 (m, 1H), 3.82 (s, 3H), 3.81 (s, 3H), 3.43-3.22 (m, 3H), 2.75-2.72 (m, 1H), 2.64 (s, 3H), 2.41 (m, 2H), 2.29 (m, 1H), 1.87 (m, 1H), 1.80 (m, 1H), 1.71 (m, 1H), 1.20 (t, J=8.0 Hz, 3H). LRMS m/z: calcd for C₃₄H₃₇ClN₆O₅ [M+H]⁺: 645.2; found 645.2.

### 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3-methoxyphenyl)pentanamide (BRD-N22c-int)

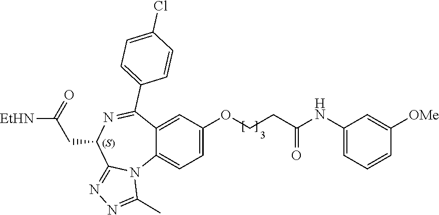

5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3-methoxyphenyl)pentanamide, (BRD-E22c) was synthesized by following the method of general EDC coupling of 3-methoxyaniline (37 mg, 0.29 mmol) and 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid, 54 (100 mg, 0.20 mmol). 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3-methoxyphenyl)pentanamide, BRD-E22c (60 mg, 50%) was isolated by flash chromatography.

### (3-(5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanamido)phenyl)boronic acid (BRD-E20)

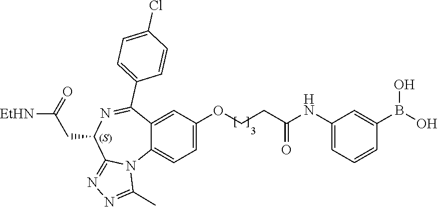

(3-(5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanamido)phenyl)boronic acid (BRD-E20) was synthesized by following the method of general EDC coupling of (3-aminophenyl)boronic acid (41 mg, 0.29 mmol) and 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid, 54 (100 mg, 0.20 mmol). (3-(5-(((4S)-6-(4-chlorophenyl)-4-(2-(ethyl amino)-2-oxoethyl)-1-methyl-4H-benzo[/] [1,2,4]triazolo[4,3-a] [1,4]diazepin-8-yl)oxy)pentanamido)phenyl)boronic acid, BRD-E20 (20 mg, 16.2%) was isolated by flash chromatography. ¹H-NMR (400 MHz, CD₃OD): δ 8.36 (s, 1H), 7.77 (s, 2H), 7.70 (d, J=9.2 Hz, 1H), 7.69-7.60 (m, 2H), 7.56-7.52 (m, 2H), 7.44-7.37 (m, 3H), 7.34-7.30 (m, 3H), 6.92 (d, J=2.8 Hz, 1H), 4.63 (q, J=5.2 Hz, 1H), 4.09-4.04 (m, 1H), 3.43-3.32 (m, 1H), 3.28-3.23 (m, 2H), 2.75 (t, J=6.8 Hz, 1H), 2.63 (s, 3H), 2.47-2.38 (m, 3H), 1.88 (m, 3H), 1.83-1.77 (m, 2H), 1.27 (t, J=7.2 Hz, 3H). LRMS m/z: calcd for C₃₂H₃₄BClN₆O₅ [M+H]⁺: 629.2; found 629.4.

### 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-phenylpentanamide (BRD-E20c)

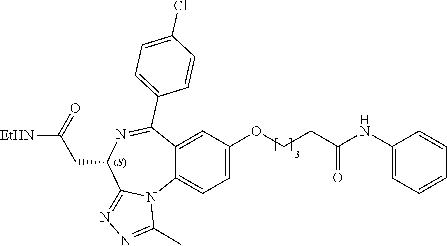

5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-phenylpentanamidez (BRD-E20c) was synthesized by following the method of general EDC coupling of aniline (28 mg, 0.29 mmol) and 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)pentanoic acid, 54 (100 mg, 0.20 mmol). 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-phenylpentanamidez, BRD-E20c (8 mg, 7%) was isolated by flash chromatography. ¹H-NMR (400 MHz, CD₃OD): δ 7.71 (d, J=9.2 Hz, 1H), 7.56-7.52 (m, 4H), 7.44-7.37 (m, 3H), 7.32-7.28 (m, 2H), 7.11-7.07 (m, 1H), 6.93 (d, J=3.2 Hz, 1H), 4.63 (q, J=5.2 Hz, 1H), 4.09-4.05 (m, 2H), 3.44-3.38 (m, 2H), 3.30-3.23 (m, 4H), 2.65 (s, 3H), 2.40 (s, 2H), 1.90-1.70 (m, 2H), 1.21 (t, J=7.6 Hz, 3H). LRMS m/z: calcd for C₃₂H₃₃ClN₆O₃ [M+H]⁺: 585.2; found 585.2.

### General Procedure for BBr₃ Mediated Demethylation

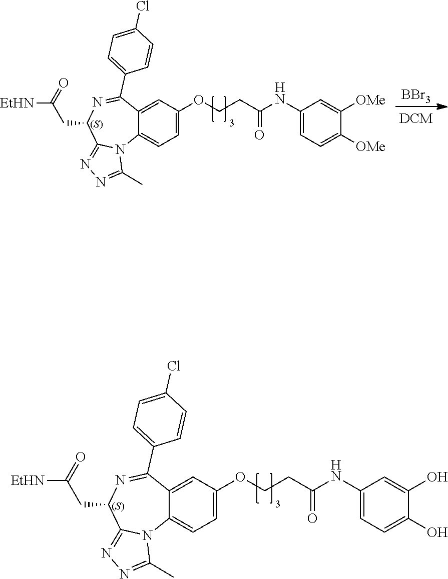

To a stirred, −78° C. solution of mono- or dimethoxy intermediate (1 eq.) in dry dichloromethane (5 mL) was added BBr₃ (1M solution in DCM, 5 equiv.), under a nitrogen atmosphere. The resulting mixture was warmed to ambient temperature and stirred for 18 h. At that point, it was cooled to 0° C., quenched with saturated aqueous sodium dithionite (10 mL), and extracted with ethyl acetate (3×20 mL). The combined organic layers were dried over anhydrous sodium sulphate, filtered, and concentrated under reduced pressure. The product was purified by preparative HPLC [column: X-Select C18 (19×150 mm, 5 m); mobile phase A: 0.1% formic acid in water; mobile phase B: ACN; flowrate: 15 mL/min]; fractions containing the product were combined and lyophilized.

### 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)pentanamide (BRD-N22)

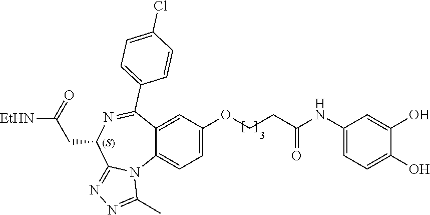

5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)pentanamide (BRD-N22) was synthesized by following the general method for BBr₃ mediated demethylation of 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dimethoxyphenyl)pentanamide, BRD-N22d (100 mg, 0.16 mmol) with BBr₃ (1M solution in DCM, 0.46 mL, 0.46 mmol). 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3,4-dihydroxyphenyl)pentanamide, BRD-N22 (3.3 mg, 3.4%) was isolated by preparative HPLC. ¹H-NMR (400 MHz, CD₃OD): δ 7.70 (d, J=9.2 Hz, 1H), 7.54 (d, J=8.8 Hz, 2H), 7.43-7.36 (m, 3H), 7.09 (d, J=2.4 Hz, 1H), 6.91 (d, J=2.8 Hz, 1H), 6.77-6.74 (m, 1H), 6.68 (d, J=8.8 Hz, 1H), 4.63 (q, J=6.4 Hz, 1H), 4.07 (m, 2H), 3.44-3.38 (m, 1H), 3.28-3.23 (m, 2H), 2.65 (s, 2H), 2.40 (m, 2H), 1.87 (m, 4H), 1.20 (t, J=7.2 Hz, 3H). LRMS m/z: calcd for C₃₂H₃₃ClN₆O₅ [M+H]⁺: 617.2; found 617.2.

### 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3-hydroxyphenyl)pentanamide (BRD-N22c)

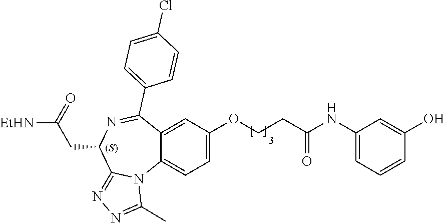

5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3-hydroxyphenyl)pentanamide, (BRD-N22c) was synthesized by following the general method for BBr₃ mediated demethylation of 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3-methoxyphenyl)pentanamide, BRD-N22c-int (60 mg, 0.10 mmol) with BBr₃ (1M solution in DCM, 0.2 mL, 0.2 mmol). 5-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-N-(3-hydroxyphenyl)pentanamide, BRD-N22c (10 mg, 17%) was isolated by preparative HPLC. ¹H-NMR (400 MHz, CD₃OD): δ 7.70 (d, J=9.2 Hz, 1H), 7.54 (d, J=8.4 Hz, 2H), 7.43-7.37 (m, 3H), 7.15 (t, J=2.4 Hz, 1H), 7.09 (t, J=8.0 Hz, 1H), 6.93-6.91 (m, 2H), 6.54-6.51 (m, 1H), 4.63 (q, J=5.2 Hz, 1H), 4.10-4.03 (m, 2H), 3.44-3.37 (m, 1H), 3.30-3.23 (m, 3H), 2.64 (s, 3H), 2.18 (m, 2H), 1.87 (m, 4H), 1.21 (t, J=7.2 Hz, 3H). LRMS m/z: calcd for C₃₂H₃₃ClN₆O₄ [M+H]f: 601.2; found 601.2.

### SYNTHESIS OF BRD-N38, BRD-N38C, BRD-N39, BRD-N39c

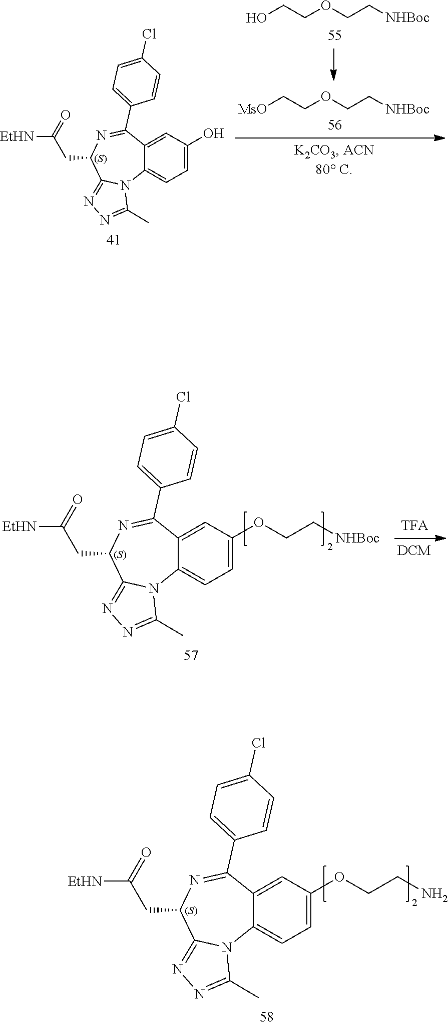

BRD-N38, BRD-N38c, BRD-N39, BRD-N39c were synthesized using processes disclosed in WO2015081280 to Arnold et al., which is hereby incorporated by reference in its entirety.

### 2-(2-((tert-butoxycarbonyl)amino)ethoxy)ethyl methanesulfonate (56)

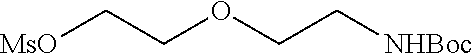 [SMILES: CC(C)(C)OC(=O)NCCOCCOS(C)(=O)=O]

To a stirred, 0° C. solution of tert-butyl (2-(2-hydroxyethoxy)ethyl)carbamate, 55 (500 mg, 2.44 mmol) in dry dichloromethane (10 mL), was added triethylamine (0.7 mL, 4.88 mmol) and mesyl chloride (0.25 mL, 3.17 mmol) under a nitrogen atmosphere. The reaction mixture was warmed to ambient temperature and stirred for 5 hours, at which point it was diluted with dichloromethane (20 mL), then washed with water (10 mL) and brine (10 ml). The organic laver was dried over anhydrous sodium sulphate, filtered, and concentrated under reduced pressure to afford 2-(2-((tert-butoxycarbonyl)amino)ethoxy)ethyl methanesulfonate, 56 (700 mg, quantitative), which was taken on without further purification.

### tert-butyl (2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)carbamate (57)

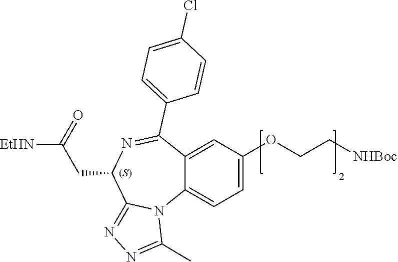

To a stirred solution of 2-((4S)-6-(4-chlorophenyl)-8-hydroxy-1-methyl-4H-benzo[f][1,2,4]triazolo[4,3-a][1,4]diazepin-4-yl)-N-ethylacetamide, 41 (500 mg, 1.22 mmol) in dry DMF (3 mL) and acetonitrile (10 mL) was added potassium carbonate (202 mg, 1.46 mmol) and 2-(2-((tert-butoxycarbonyl)amino)ethoxy)ethyl methanesulfonate, 56 (415 mg, 1.46 mmol). The resulting mixture was heated at 80° C. for 4 h, at which point it was diluted with ethyl acetate (30 mL), then washed with ice water (10 mL) and brine (10 mL). The organic layer was separated, dried over anhydrous sodium sulphate, filtered, and concentrated under reduced pressure then purified by flash chromatography (60-120 mesh, 8-10% MeOH in DCM). Fractions containing the desired product were concentrated under reduced pressure to afford tert-butyl (2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)carbamate, 57 (400 mg, 55%).

### 2-((4S)-8-(2-(2-aminoethoxy)ethoxy)-6-(4-chlorophenyl)-1-methyl-4H-benzo[f][1,2,4]triazolo[4,3-a][1,4]diazepin-4-yl)-N-ethylacetamide (58)

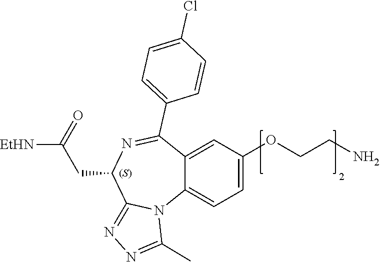

To a stirred, ambient temperature solution of tert-butyl (2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)carbamate, 57 (400 mg, 0.67 mmol) in dichloromethane (20 mL) under nitrogen atmosphere was added trifluoroacetic acid (1 mL). The resulting mixture was stirred at ambient temperature for 18 h, at which point it was concentrated under reduced pressure and triturated with diethyl ether (2×10 mL) to obtain 2-((4S)-8-(2-(2-aminoethoxy)ethoxy)-6-(4-chlorophenyl)-1-methyl-4H-benzo[f][1,2,4]triazolo[4,3-a][1,4]diazepin-4-yl)-N-ethylacetamide (TFA salt), 58 (300 mg, 90.9%) as a pale-yellow solid, which was taken on without any further purification.

### General Procedure (II) for HATU Coupling

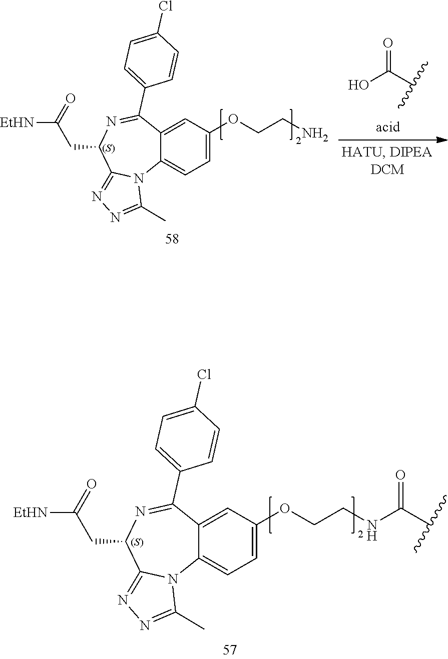

To a well-stirred solution of carboxylic acid (1.5 eq.) in dichloromethane at ambient temperature was added DIPEA (2 eq.) and HATU (1.5 eq.) under a nitrogen atmosphere. The resulting mixture was stirred at ambient temperature for 15 min at which point 2-((4S)-8-(2-(2-aminoethoxy)ethoxy)-6-(4-chlorophenyl)-1-methyl-4H-benzo[f][1,2,4]triazolo[4,3-a][1,4]diazepin-4-yl)-N-ethylacetamide (TFA salt), 58 (1 eq.) was added. The resulting solution was stirred for an additional 18 h. Upon completion, the reaction was cooled and diluted with DCM (10 mL) and then washed sequentially with water (5 mL), and brine (5 mL). The organic layer was separated, dried over anhydrous sodium sulphate, filtered, and concentrated under reduced pressure. The reaction was purified by preparative HPLC [column: X-Select C18 (19×150 mm, 5 μm); mobile phase A: 0.1% formic acid in water; mobile phase B: ACN; flowrate: 15 mL/min]. Fractions containing the product were combined and lyophilized.

### N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-3,4-dihydroxybenzamide (BRD-N38)

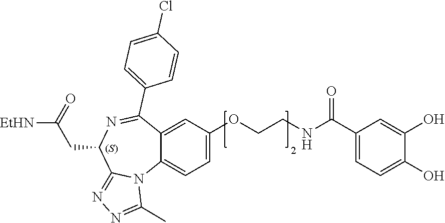

N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-3,4-dihydroxybenzamide (BRD-N38) was synthesized by following the method of general HATU coupling of 3,4-dihydroxybenzoic acid (47 mg, 0.30 mmol) and 58 (100 mg, 0.20 mmol). N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-3,4-dihydroxybenzamide, BRD-N38 (8 mg, 6.3%) was isolated following preparative HPLC as an off-white solid. ¹H-NMR (400 MHz, CD₃OD): δ 8.55 (s, 1H), 7.64 (d, J=8.8 Hz, 1H), 7.53-7.50 (m, 1H), 7.42-7.40 (m, 2H), 7.37-7.34 (m, 1H), 7.24 (d, J=2.0 Hz, 1H), 7.17-7.15 (m, 1H), 6.90 (d, J=2.8 Hz, 1H), 6.75 (d, J=8.4 Hz, 1H), 4.64 (q, J=5.2 Hz, 1H), 4.18-4.15 (m, 2H), 3.85 (t, J=4.0 Hz, 2H), 3.71 (t, J=5.2 Hz, 2H), 3.55-3.53 (m, 3H), 3.51-3.50 (m, 1H), 3.33-3.32 (m, 2H), 2.64 (s, 3H), 1.21 (t, J=7.2 Hz, 3H). LRMS m/z: calcd for C₃₂H₃₃ClN₆O₆ [M+H]⁺: 633.2; found 633.2.

### N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)benzamide (BRD-N38c)

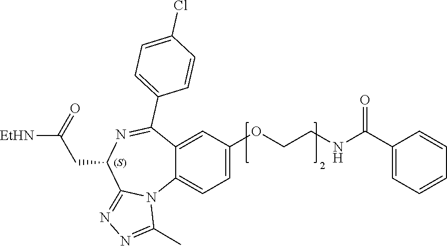

N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)benzamide, (BRD-N38c) was synthesized by following the method of general HATU coupling of benzoic acid (22 mg, 0.20 mmol) and 58 (50 mg, 0.11 mmol). N-(2-(2-(((4S)-6-(4-chlorophenyl)-4-(2-(ethylamino)-2-oxoethyl)-1-methyl-4H-benzo[f][f1,2,4]triazolo[4,3-a][1,4]diazepin-8-yl)oxy)ethoxy)ethyl)benzamide, BRD-N38c (12 mg, 19.8%) was isolated following preparative HPLC as a white solid. ¹H-NMR (400 MHz, CD₃OD): δ 7.81-7.78 (m, 2H), 7.67 (d, J=8.8 Hz, 1H), 7.55-7.50 (m, 3H), 7.44-7.38 (m, 5H), 6.93 (d, J=2.8 Hz, 1H), 4.62 (q, J=5.2 Hz, 1H), 4.20-4.17 (m, 2H), 3.87-3.84 (m, 2H), 3.73 (t, J=5.6 Hz, 2H), 3.61-3.57 (m, 2H), 3.44-3.38 (m, 1H), 3.30-3.27 (m, 3H), 2.64 (s, 3H), 1.21 (t, J=7.2 Hz, 3H). LRMS m/z: calcd for C₃₂H₃₃ClN₆O₄ [M+H]⁺: 601.2; found 601.2.

### N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-2,3-dihydroxybenzamide (BRD-N39)

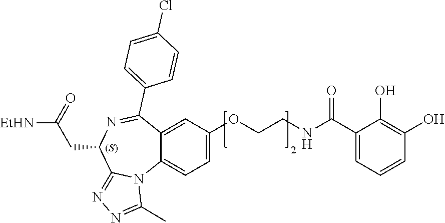

N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-2,3-dihydroxybenzamide (BRD-N39) was synthesized by following the method of general HATU coupling of 2,3-dihydroxybenzoic acid (47 mg, 0.30 mmol) and 58 (100 mg, 0.20 mmol). N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-2,3-dihydroxybenzamide, BRD-N39 (20 mg, 15.7%) was isolated following preparative HPLC as a white solid. ¹H-NMR (400 MHz, CD₃OD): δ 7.64 (d, J=9.2 Hz, 1H), 7.55-7.53 (m, 2H), 7.43-7.36 (m, 3H), 7.21-7.18 (m, 1H), 6.93-6.88 (m, 2H), 6.67 (t, J=8.0 Hz, 1H), 4.67 (q, J=5.6 Hz, 1H), 4.20-4.18 (m, 2H), 3.87-3.86 (m, 2H), 3.75-3.72 (m, 2H), 3.61-3.57 (m, 2H), 3.43-3.39 (m, 1H), 3.33-3.28 (m, 3H), 2.74 (s, 3H), 1.20 (t, J=5.2 Hz, 3H). LRMS m/z: calcd for C₃₂H₃₃ClN₆O₆ [M+H]⁺: 633.2; found 633.2.

### N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-2-hydroxybenzamide (BRD-N39c)

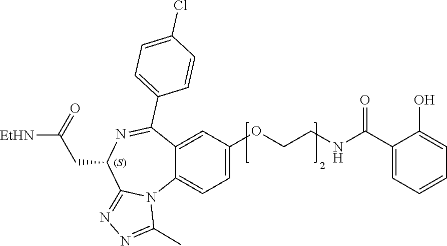

N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-2-hydroxybenzamide (BRD-N39c) was synthesized by following the method of general HATU coupling of 2-hydroxybenzoic acid (27 mg, 0.20 mmol) and 58 (50 mg, 0.11 mmol). N-(2-(2-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)ethoxy)ethyl)-2-hydroxybenzamide, BRD-N39c (5 mg, 7%) was isolated following preparative HPLC as a white solid. ¹H-NMR (400 MHz, CD₃OD): δ 7.74 (dd, J=8.4, 1.6 Hz, 1H), 7.62 (d, J=9.2 Hz, 1H), 7.53-7.51 (m, 2H), 7.42-7.39 (m, 3H), 7.37-7.32 (m, 1H), 6.93 (d, J=2.8 Hz, 1H), 6.87-6.82 (m, 2H), 4.61 (q, J=5.2 Hz, 1H), 4.20-4.17 (m, 2H), 3.86 (t, J=4.0 Hz, 2H), 3.73 (t, J=5.6 Hz, 2H), 3.62-3.56 (m, 2H), 3.41 (q, J=8.8 Hz, 1H), 3.28-3.23 (m, 3H), 2.63 (s, 3H), 1.21 (t, J=7.2 Hz, 3H) LRMS m/z: calcd for C₃₂H₃₃ClN₆O₅ [M+H]⁺: 617.2; found 617.3.

### Synthesis of BRD-N70, BRD-N70C, BRD-N71 and BRD-N71c

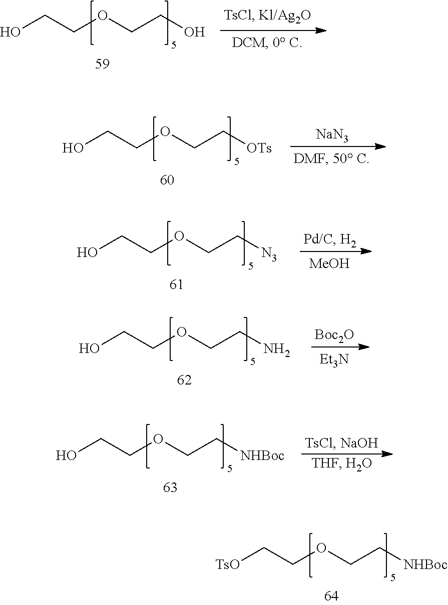

BRD-N70, BRD-N70c, BRD-N71 and BRD-N71c were synthesized using processes disclosed in WO2015081280 to Arnold et al., and Mollet et al., J. Mater. Chem. B, 2 (17), 2483-2493 (2014), which are hereby incorporated by reference in their entirety.

### 17-hydroxy-3,6,9,12,15-pentaoxaheptadecyl 4-methylbenzenesulfonate (60)

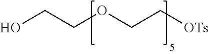

To a stirred, 0° C. solution of 3,6,9,12,15-pentaoxaheptadecane-1,17-diol, 59 (2 g, 7.08 mmol) in dry dichloromethane (25 mL) was added silver(I) oxide (2.46 g, 10.62 mmol), p-toluenesulfonyl chloride (1.48 g, 7.79 mmol) and KI (235 mg, 1.42 mmol) under an atmosphere of nitrogen. The resulting mixture was warmed to ambient temperature and stirred for 2 h. At that point, the solution was filtered through a pad of Celite and concentrated under reduced pressure. The remaining residue was purified by flash chromatography (60-120 mesh, 8-10% MeOH in DCM). Fractions containing the desired product were combined and concentrated under reduced pressure to afford 17-hydroxy-3,6,9,12,15-pentaoxaheptadecyl 4-methylbenzenesulfonate, 60 (2.9 g, 93.8%) as a colorless oil.

### 17-azido-3,6,9,12,15-pentaoxaheptadecan-1-ol (61)

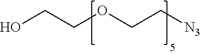

To a stirred solution of 17-hydroxy-3,6,9,12,15-pentaoxaheptadecyl 4-methylbenzenesulfonate, 60 (2.9 g, 6.64 mmol) in dry DMF (20 mL) was added sodium azide (648 mg, 9.96 mmol) under an atmosphere of nitrogen. The resulting solution was heated to 50° C. and stirred for 8 h, at which point it was cooled to ambient temperature and quenched with ice-water (20 mL) and extracted with dichloromethane (3×25 mL). The combined organic layers were washed with brine (25 mL), dried over anhydrous sodium sulfate, filtered, and concentrated under reduced pressure. The remaining residue was purified by flash chromatography (60-120 mesh, 50-100% EtOAc in petroleum ether). Fractions containing the desired product were combined and concentrated under reduced pressure to afford 17-azido-3,6,9,12,15-pentaoxaheptadecan-1-ol, 61 (1.4 g, 68.6%) as a pale-yellow liquid.

### 17-amino-3,6,9,12,15-pentaoxaheptadecan-1-ol (62)

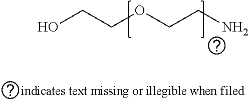

To a stirred solution of 17-azido-3,6,9,12,15-pentaoxaheptadecan-1-ol 61 (1.4 g, 4.56 mmol) in dry methanol (20 mL) was added palladium on carbon (200 mg, 10% wt.) and 25% aqueous ammonia (5 mL). The resulting mixture was stirred at ambient temperature under H₂ balloon pressure for 5 h, and then filtered through a bed of Celite. The Celite bed was washed with methanol (2×25 mL), and the combined filtrates were concentrated under reduced pressure to afford 17-amino-3,6,9,12,15-pentaoxaheptadecan-1-ol, 62 (1 g, 78%) as a colorless liquid, which was used without further purification.

### tert-butyl (17-hydroxy-3,6,9,12,15-pentaoxaheptadecyl)carbamate (63)

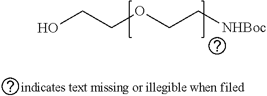

To a stirred solution of 17-amino-3,6,9,12,15-pentaoxaheptadecan-1-ol, 62 (1 g, 3.55 mmol) in dry methanol (25 mL) was added triethylamine (0.6 mL, 4.26 mmol) and Boc anhydride (853 mg, 3.91 mmol). The resulting solution was stirred at ambient temperature for 18 h and then concentrated under reduced pressure to afford tert-butyl (17-hydroxy-3,6,9,12,15-pentaoxaheptadecyl)carbamate, 63 (1.4 g, 99%) as a colorless liquid, which was used without further purification.

### 2,2-dimethyl-4-oxo-3,8,11,14,17,20-hexaoxa-5-azadocosan-22-yl 4-methylbenzenesulfonate (64)

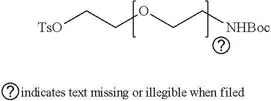

To a stirred, 0° C. solution of tert-butyl (17-hydroxy-3,6,9,12,15-pentaoxaheptadecyl)carbamate, 63 (1.4 g, 3.67 mmol) in dry THF (20 mL) was added sodium hydroxide (294 mg, 7.34 mmol) and p-toluenesulfonyl chloride (840 mg, 4.40 mmol) under an atmosphere of nitrogen. The resulting solution was warmed to ambient temperature and stirred for 18 h, then concentrated under reduced pressure. The residue was purified by flash chromatography (60-120 mesh, 8-10% MeOH in DCM). Fractions containing the desired product were combined and concentrated under reduced pressure to afford 2,2-dimethyl-4-oxo-3,8,11,14,17,20-hexaoxa-5-azadocosan-22-yl 4-methylbenzenesulfonate, 64 (1.2 g, 61.2%) as a colorless liquid.

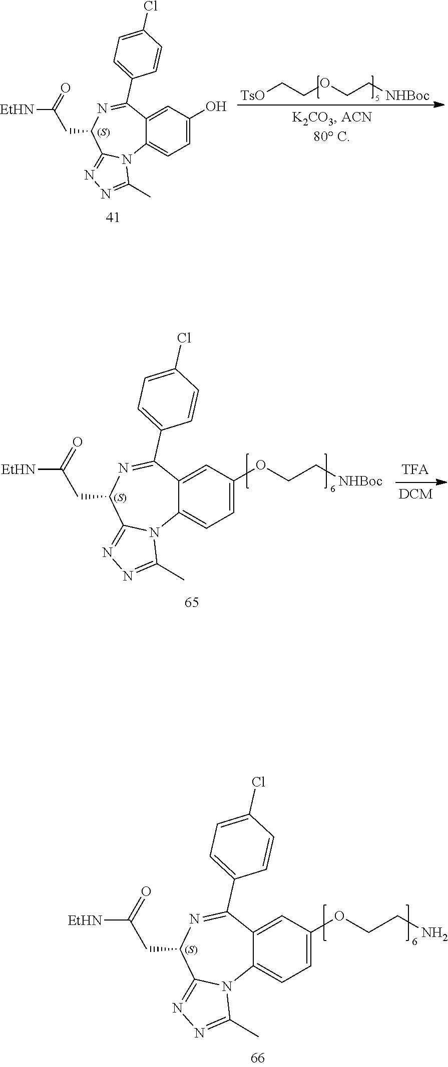

### tert-butyl (17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)carbamate (65)

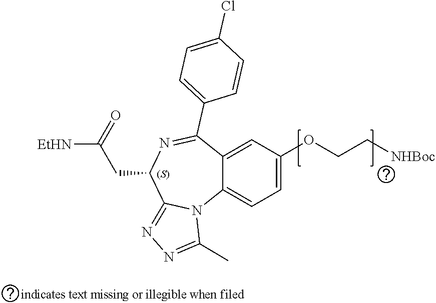

To a stirred solution of (2-((4S)-6-(4-chlorophenyl)-8-hydroxy-1-methyl-4H-benzo[f][1,2,4]triazolo[4,3-a][1,4]diazepin-4-yl)-N-ethylacetamide, 41 (600 mg, 1.46 mmol) in dry acetonitrile (20 mL) was added potassium carbonate (303 mg, 2.20 mmol) and 2,2-dimethyl-4-oxo-3,8,11,14,17,20-hexaoxa-5-azadocosan-22-yl 4-methylbenzenesulfonate, 64 (940 mg, 1.76 mmol) under an atmosphere of nitrogen. The resulting mixture was heated to 90° C. and stirred for 18 h, at which point it was cooled to ambient temperature and diluted with ethyl acetate (30 mL), then washed with ice-water (10 mL) and brine (10 ml). The organic layer was separated, dried over anhydrous sodium sulphate, filtered, and concentrated under reduced pressure. The remaining residue was purified by flash chromatography (60-120 mesh, 8-10% MeOH in DCM). Fractions containing the desired product were combined and concentrated under reduced pressure to afford tert-butyl (17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)carbamate, 65 (730 mg, 64.6%).

### 2-((4S)-8-((17-amino-3,6,9,12,15-pentaoxaheptadecyl)oxy)-6-(4-chlorophenyl)-1-methyl-4H-benzo[f][1,2,4]triazolo[4,3-a][1,4]diazepin-4-yl)-N-ethylacetamide (66)

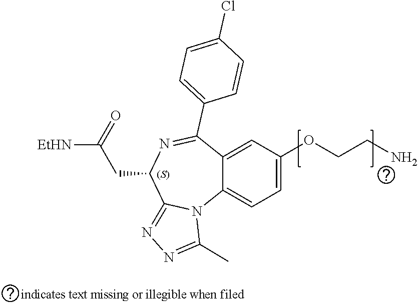

To a stirred, ambient temperature solution of tert-butyl (17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)carbamate, 65 (730 mg, 0.94 mmol) in dichloromethane (10 mL) under nitrogen atmosphere was added trifluoroacetic acid (2 mL). The resulting mixture was stirred at ambient temperature for 18 h, at which point it was concentrated under reduced pressure and triturated with diethyl ether (2×10 mL) to obtain 2-((4S)-8-((17-amino-3,6,9,12,15-pentaoxaheptadecyl)oxy)-6-(4-chlorophenyl)-1-methyl-4H-benzo[f][1,2,4]triazolo[4,3-a][1,4]diazepin-4-yl)-N-ethylacetamide (TFA salt), 66 (600 mg, 94%) as a brown, gummy solid, which was taken on without any further purification.

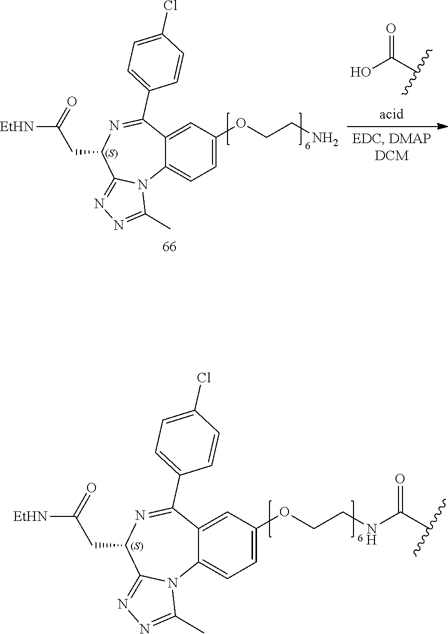

### N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-3,4-dihydroxybenzamide (BRD-N70)

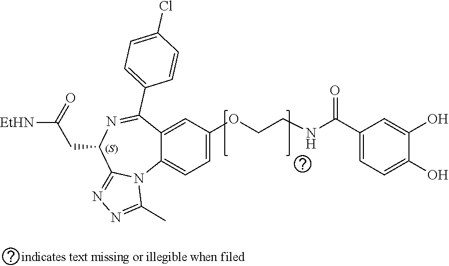

N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-3,4-dihydroxybenzamide (BRD-N70) was synthesized by following the method of general EDC coupling of 3,4-dihydroxybenzoic acid (17 mg, 0.11 mmol) and 2-((4S)-8-((17-amino-3,6,9,12,15-pentaoxaheptadecyl)oxy)-6-(4-chlorophenyl)-1-methyl-4H-benzo[f][1,2,4]triazolo[4,3-a][1,4]diazepin-4-yl)-N-ethylacetamide (TFA salt), 66 (50 mg, 0.07 mmol). N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-3,4-dihydroxybenzamide, BRD-N70 (15 mg, 25%) was isolated by preparative HPLC. ¹H-NMR (400 MHz, CD₃OD): δ 7.71 (m, 1H), 7.56-7.53 (m, 2H), 7.44-7.37 (m, 3H), 7.29 (t, J=1.2 Hz, 1H), 7.22 (q, J=2.4 Hz, 1H), 6.94 (d, J=3.2 Hz, 1H), 6.79 (d, J=8.0 Hz, 1H), 4.65 (m, 1H), 4.2-4.1 (m, 2H), 3.8 (m, 2H), 3.66-3.59 (m, 19H), 3.52 (m, 3H), 3.33-3.28 (m, 2H), 2.65 (s, 3H), 1.20 (t, J=7.2 Hz, 3H). LRMS m/z: calcd for C₄₀H₄₉ClN₆O₁₀[M+H]⁺: 809.2; found 809.2.

### N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-3-hydroxybenzamide (BRD-N70c)

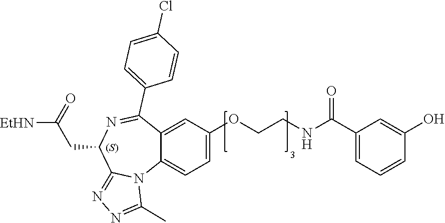

N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-3-hydroxybenzamide (BRD-N70c) was synthesized by following the method of general EDC coupling of 3-hydroxybenzoic acid (46 mg, 0.33 mmol) and 2-((4S)-8-((17-amino-3,6,9,12,15-pentaoxaheptadecyl)oxy)-6-(4-chlorophenyl)-1-methyl-4H-benzo[f][1,2,4]triazolo[4,3-a][1,4]diazepin-4-yl)-N-ethylacetamide (TFA salt), 66 (150 mg, 0.22 mmol). N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-3-hydroxybenzamide, BRD-N70c (20 mg, 11.3%) was isolated by preparative HPLC. ¹H-NMR (400 MHz, CD₃OD): δ 8.37 (brs, 2H), 7.71 (d, J=8.8 Hz, 1H), 7.57-7.54 (m, 2H), 7.44-7.38 (m, 3H), 7.26-7.23 (m, 3H), 6.96-6.91 (m, 2H), 4.64 (q, J=5.2 Hz, 1H), 4.16-4.14 (m, 2H), 3.83 (t, J=4.4 Hz, 2H), 3.65-3.56 (m, 21H), 3.39 (m, 1H), 3.33-3.25 (m, 3H), 2.65 (s, 3H), 1.20 (t, J=7.2 Hz, 3H). LRMS m/z: calcd for C₄₀H₄₉ClN₆O₉ [M+H]⁺: 793.2; found 793.2.

### N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-2,3-dihydroxybenzamide (BRD-N71)

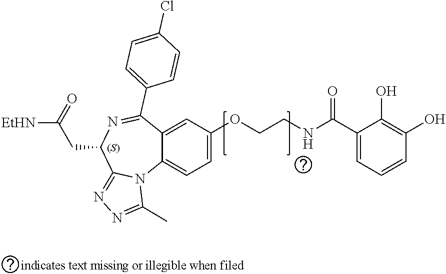

N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-2,3-dihydroxybenzamide (BRD-N71) was synthesized by following the method of general EDC coupling of 2,3-dihydroxybenzoic acid (51 mg, 0.33 mmol) and 2-((4S)-8-((17-amino-3,6,9,12,15-pentaoxaheptadecyl)oxy)-6-(4-chlorophenyl)-1-methyl-4H-benzo[f][1,2,4]triazolo[4,3-a][1,4]diazepin-4-yl)-N-ethylacetamide (TFA salt), 66 (150 mg, 0.22 mmol). N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)-2,3-dihydroxybenzamide, BRD-N71 (30 mg, 16.6%) was isolated by preparative HPLC. ¹H-NMR (400 MHz, CD₃OD): δ 7.69 (d, J=8.8 Hz, 1H), 7.56-7.54 (m, 2H), 7.43-7.37 (m, 3H), 7.24 (dd, J=8.0, 1.6 Hz, 1H), 6.95-6.90 (m, 2H), 6.71 (t, J=8.0 Hz, 1H), 4.65 (q, J=5.2 Hz, 1H), 4.17-4.12 (m, 2H), 3.83 (q, J=4.4 Hz, 2H), 3.64-3.58 (m, 20H), 3.41 (m, 1H), 3.33-3.25 (m, 3H), 2.64 (s, 3H), 1.20 (t, J=7.2 Hz, 3H). LRMS m/z: calcd for C₄₀H₄₉ClN₆O₁₀ [M+H]⁺: 809.2; found 809.2.

### N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)benzamide (BRD-N71c)

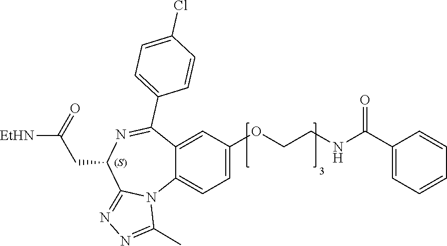

N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)benzamide (BRD-N71c) was synthesized by following the method of general EDC coupling of benzoic acid (37 mg, 0.33 mmol) and 2-((4S)-8-((17-amino-3,6,9,12,15-pentaoxaheptadecyl)oxy)-6-(4-chlorophenyl)-1-methyl-4H-benzo[f][1,2,4]triazolo[4,3-a][1,4]diazepin-4-yl)-N-ethylacetamide (TFA salt), 66 (150 mg, 0.22 mmol). N-(17-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)-3,6,9,12,15-pentaoxaheptadecyl)benzamide, BRD-N71c (70 mg, 40.5%) was isolated by preparative HPLC. ¹H-NMR (400 MHz, CD₃OD): δ 8.48 (brs, 1H), 8.37 (brs, 1H), 8.17 (s, 1H), 7.83 (dd, J=8.0, 1.2 Hz, 2H), 7.72 (d, J=8.8 Hz, 1H), 7.58-7.51 (m, 3H), 7.47-7.39 (m, 5H), 6.96 (d, J=2.8 Hz, 1H), 4.64 (q, J=5.2 Hz, 1H), 4.17-4.13 (m, 2H), 3.84-3.81 (m, 2H), 3.68-3.58 (m, 20H), 3.44-3.33 (m, 2H), 3.31-2.65 (m, 1H), 2.65 (s, 3H), 1.20 (t, J=7.2 Hz, 3H). LRMS m/z: calcd for C₄₀H₄₉ClN₆O₈ [M+H]⁺: 777.2; found 777.2.

### Synthesis of BRD-E79 and BRD-E79C

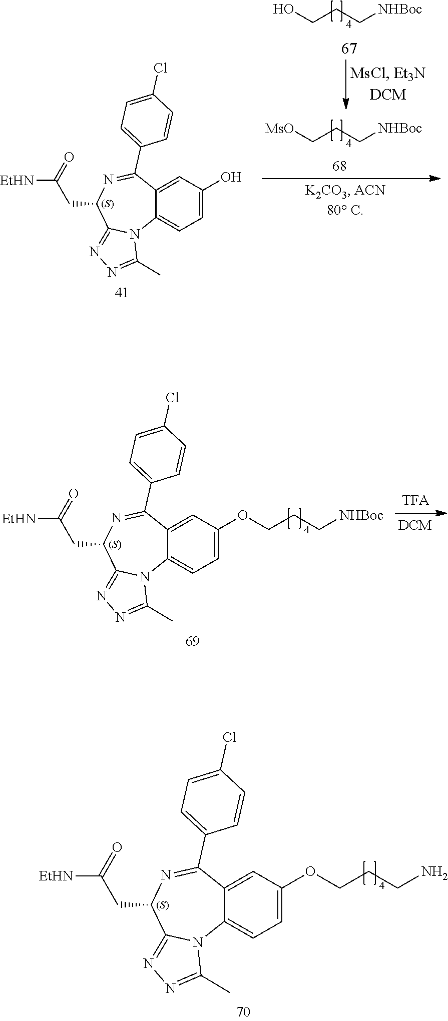

BRD-N79, and BRD-N79c, were synthesized using processes disclosed in WO2015081280, to Arnold et al., which is hereby incorporated by reference in its entirety.

### 6-((tert-butoxycarbonyl)amino)hexyl methanesulfonate (68)

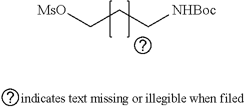

To a stirred, 0° C. solution of tert-butyl (6-hydroxyhexyl)carbamate, 67 (1 g, 4.60 mmol) in dry dichloromethane (10 mL), was added triethylamine (1.3 mL, 9.21 mmol) and mesyl chloride (0.54 mL, 6.90 mmol) under a nitrogen atmosphere. The reaction mixture was warmed to ambient temperature and stirred for 5 hours, at which point it was diluted with dichloromethane (20 mL), then washed with water (2×10 mL) and brine (10 ml). The organic layer was dried over anhydrous sodium sulphate, filtered, and concentrated under reduced pressure to afford 6-((tert-butoxycarbonyl)amino)hexyl methanesulfonate, 68 (1.3 g, 95.6%), which was taken on without further purification.

### tert-butyl (6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)carbamate (69)

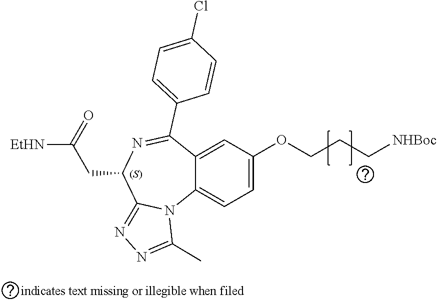

To a stirred solution of 2-((4S)-6-(4-chlorophenyl)-8-hydroxy-1-methyl-4H-benzo[f][1,2,4]triazolo[4,3-a][1,4]diazepin-4-yl)-N-ethylacetamide, 41 (300 mg, 0.73 mmol) in acetonitrile (10 mL) was added potassium carbonate (202 mg, 1.46 mmol) and 6-((tert-butoxycarbonyl)amino)hexyl methanesulfonate, 68 (330 mg, 1.09 mmol). The resulting mixture was heated at 80° C. for 18 h, at which point it was diluted with ethyl acetate (30 mL), then washed with ice water (10 mL) and brine (10 mL). The organic layer was separated, dried over anhydrous sodium sulphate, filtered, and concentrated under reduced pressure then purified by flash chromatography (60-120 mesh, 8-10% MeOH in DCM). Fractions containing the desired product were concentrated under reduced pressure to afford tert-butyl (6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)carbamate, 69 (300 mg, 67.5%).

### 2-((4S)-8-((6-aminohexyl)oxy)-6-(4-chlorophenyl)-1-methyl-4H-benzo[f][1,2,4]triazolo[4,3-a][1,4]diazepin-4-yl)-N-ethylacetamide (70)

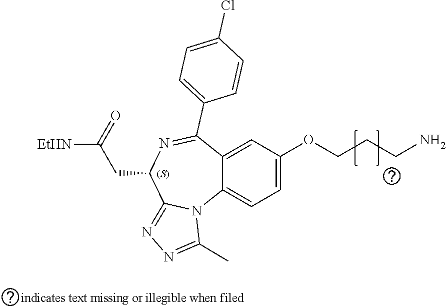

To a stirred, ambient temperature solution of tert-butyl (6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)carbamate, 57 (300 mg, 0.73 mmol) in dichloromethane (10 mL) under nitrogen atmosphere was added trifluoroacetic acid (1 mL). The resulting mixture was stirred at ambient temperature for 18 h, at which point it was concentrated under reduced pressure and triturated with diethyl ether (2×10 mL) to obtain 2-((4S)-8-((6-aminohexyl)oxy)-6-(4-chlorophenyl)-1-methyl-4H-benzo[f][1,2,4]triazolo[4,3-a][1,4]diazepin-4-yl)-N-ethylacetamide (TFA salt), 70 (300 mg) as a pale-yellow solid, which was taken on without any further purification.

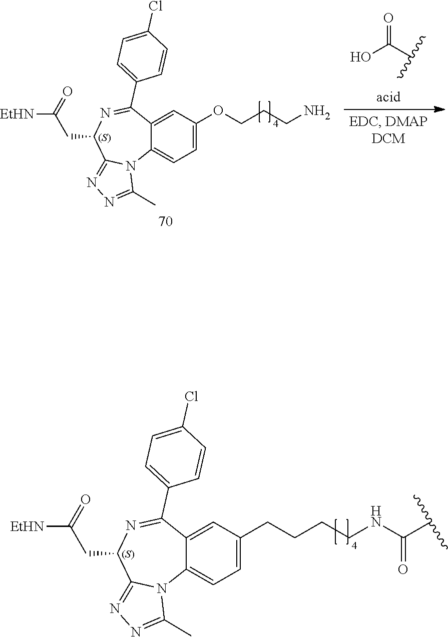

### (4-((6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)carbamoyl)phenyl)boronic acid (BRD-E79)

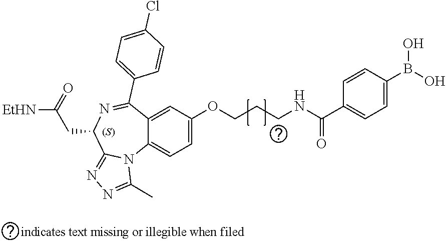

(4-((6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)carbamoyl)phenyl)boronic acid (BRD-E79) was synthesized by following the method of general EDC coupling of 4-boronobenzoic acid (81 mg, 0.59 mmol) and 2-((4S)-8-((6-aminohexyl)oxy)-6-(4-chlorophenyl)-1-methyl-4H-benzo[f][1,2,4]triazolo[4,3-a][1,4]diazepin-4-yl)-N-ethylacetamide (TFA salt), 70 (150 mg, 0.29 mmol). (4-((6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)carbamoyl)phenyl)boronic acid, BRD-E79 (7 mg, 2.7%) was isolated by preparative HPLC. ¹H-NMR (400 MHz, CD₃OD): δ 8.49 (brs, 1H), 8.37 (brs, 1H), 7.80-7.68 (m, 6H), 7.56-7.53 (m, 2H), 7.44-7.41 (m, 2H), 7.37-7.34 (q, J=2.8 Hz, 1H), 6.90 (d, J=2.8 Hz, 1H), 4.64 (q, J=5.2 Hz, 1H), 4.05-4.00 (m, 2H), 3.50-3.40 (m, 2H), 3.30-3.23 (m, 2H), 2.66 (s, 3H), 1.81 (t, J=7.6 Hz, 2H), 1.67 (t, J=7.2 Hz, 2H), 1.56-1.46 (m, 4H), 1.33 (t, J=7.2 Hz, 2H), 1.21 (t, J=7.2 Hz, 3H). LRMS m/z: calcd for C₃₄H₃₈BClN₆O₅ [M+H]⁺: 657.3; found 657.2.

### N-(6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)benzamide (BRD-E79c)

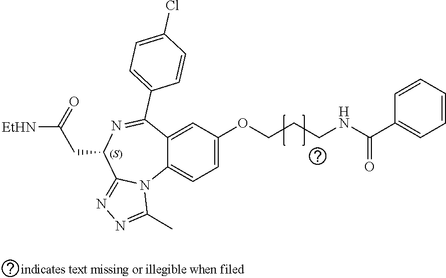

N-(6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)benzamide (BRD-N79c) was synthesized by following the method of general EDC coupling of benzoic acid (65 mg, 0.59 mmol) and 2-((4S)-8-((6-aminohexyl)oxy)-6-(4-chlorophenyl)-1-methyl-4H-benzo[f][1,2,4]triazolo[4,3-a][1,4]diazepin-4-yl)-N-ethylacetamide (TFA salt), 70 (150 mg, 0.29 mmol). N-(6-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)oxy)hexyl)benzamide, BRD-N79c (14 mg, 5.8%) was isolated by preparative HPLC. ¹H-NMR (400 MHz, CD₃OD): δ 8.43 (brs, 1H), 7.81 (d, J=1.6 Hz, 2H), 7.77-7.69 (m, 1H), 7.57-7.51 (m, 3H), 7.48-7.35 (m, 5H), 6.90 (d, J=2.8 Hz, 1H), 4.64 (q, J=5.2 Hz, 1H), 4.06-3.99 (m, 2H), 3.44-3.38 (m, 2H), 3.30-3.23 (m, 3H), 3.01 (s, 1H), 2.65 (s, 3H), 1.80 (t, J=6.4 Hz, 2H), 1.67 (t, J=7.2 Hz, 2H), 1.56-1.46 (m, 4H), 1.21 (t, J=7.2 Hz, 3H). LRMS m/z: calcd for C₃₄H₃₇ClN₆O₃ [M+H]⁺: 613.3; found 613.2.

### Synthesis of BRD-E50

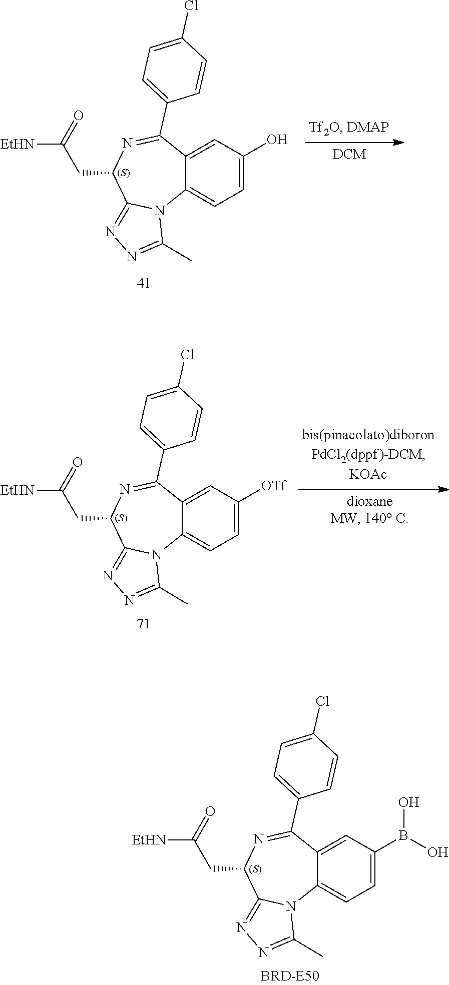

BRD-E50 was synthesized using processes disclosed in WO2015081280, to Arnold et al., which is hereby incorporated by reference in its entirety.

### (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate (71)

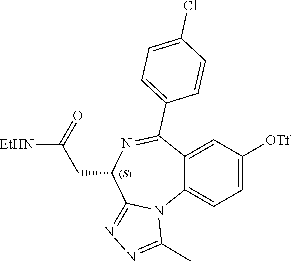

To a stirred solution of (2-((4S)-6-(4-chlorophenyl)-8-hydroxy-1-methyl-4H-benzo[f][1,2,4]triazolo[4,3-a][1,4]diazepin-4-yl)-N-ethylacetamide, 41 (400 mg, 0.98 mmol) in dry dichloromethane (10 mL) was added DMAP (179 mg, 1.46 mmol) and trifluoromethanesulfonic anhydride (0.2 mL, 1.27 mmol) under an atmosphere of nitrogen. The resulting solution was stirred at ambient temperature for 18 h, at which point it was diluted with ethyl acetate (30 mL), then washed with ice-water (10 mL) and brine (10 mL). The organic layer was separated, dried over anhydrous sodium sulphate, filtered, and concentrated under reduced pressure then purified by flash chromatography (60-120 mesh, 8-10% MeOH in DCM). Fractions containing the desired product were concentrated under reduced pressure to afford (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (150 mg, 28%).

### ((4S)-6-(4-chlorophenyl)-4-(2-(ethylamino)-2-oxoethyl)-1-methyl-4H-benzo[f][1,2,4]triazolo[4,3-a][1,4]diazepin-8-yl)boronic acid (BRD-E50)

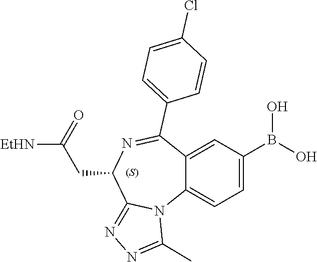

To an 8 mL microwave reaction via containing a solution of (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (90 mg, 0.17 mmol) in 1,4-dioxane (3 mL) was added bis(pinacolato)diboron (84 mg, 0.33 mmol) and potassium acetate (48 mg, 0.50 mmol). The resulting solution was purged with nitrogen for 10 min, at which point Pd(dppf)Cl₂·DCM (80 mg, 0.11 mmol) was added and the resulting mixture was heated at 140° C. under microwave irradiation for 30 min, then cooled to ambient temperature and concentrated under reduced pressure. The resulting mixture was purified by preparative HPLC [column: X-Select C18 (19×150 mm, 5 m); mobile phase A: 0.1% formic acid in water; mobile phase B: ACN; flowrate: 15 mL/min]. Fractions containing the product were combined and lyophilized to afford ((4S)-6-(4-chlorophenyl)-4-(2-(ethylamino)-2-oxoethyl)-1-methyl-4H-benzo[f][1,2,4]triazolo[4,3-a][1,4]diazepin-8-yl)boronic acid, BRD-E50 (10 mg, 13%) as an off-white solid. ¹H-NMR (400 MHz, CD₃OD): δ 8.37 (s, 1H), 8.13 (brs, 1H), 7.78 (d, J=8.4 Hz, 2H), 7.55-7.15 (m, 2H), 7.44-7.40 (m, 2H), 4.65-4.60 (m, 1H), 3.74 (m, 1H), 3.42-3.35 (m, 1H), 3.31-3.26 (m, 2H), 2.69 (s, 3H), 1.20 (t, J=7.2 Hz, 3H). LRMS m/z: calcd for C₂₁H₂₁BClN₅O₃ [M+H]⁺: 438.1; found 438.0.

### Synthesis of BRD-E72 and BRD-E72c

BRD-E72 and BRD-E72c were synthesized using processes disclosed in WO2015081280 to Arnold et al., and WO2011161031 to Bailey, which are hereby incorporated by reference in their entirety.

### General Procedure for Suzuki Coupling

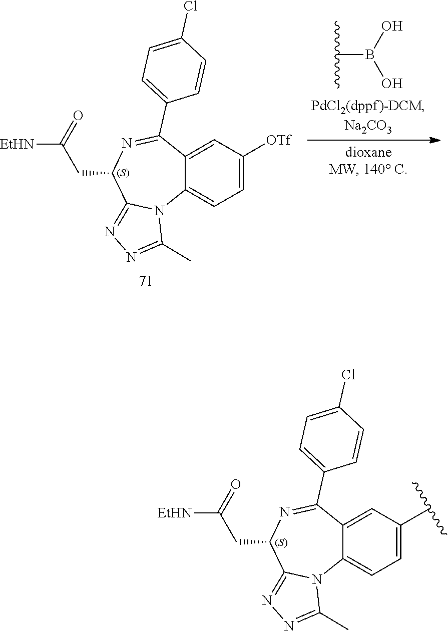

To an 8 mL microwave reaction vial containing a solution of (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (1 eq) in 1,4-dioxane (3 mL) was added boronic acid (1.8 eq) and sodium carbonate (2.5 eq). The resulting solution was purged with nitrogen for 10 min, at which point Pd(dppf)Cl₂·DCM (0.15 eq) was added and the resulting mixture was heated at 140° C. under microwave irradiation for 30 min, then cooled to ambient temperature and concentrated under reduced pressure. The resulting mixture was purified by preparative HPLC [column: X-Select C18 (19×150 mm, 5 μm); mobile phase A: 0.1% formic acid in water; mobile phase B: ACN; flowrate: 15 mL/min]. Fractions containing the product were combined and lyophilized.

### (3-((4S)-6-(4-chlorophenyl)-4-(2-(ethylamino)-2-oxoethyl)-1-methyl-4H-benzo[f][1,2,4]triazolo[4,3-a][1, 4]diazepin-8-yl)phenyl)boronic acid (BRD-E 72)

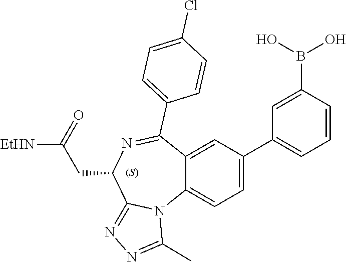

(3-((4S)-6-(4-chlorophenyl)-4-(2-(ethylamino)-2-oxoethyl)-1-methyl-4H-benzo[f][1,2,4]triazolo[4,3-a][1,4]diazepin-8-yl)phenyl)boronic acid (BRD-N72) was synthesized by following the procedure for the Suzuki coupling 1,3-phenylenediboronic acid (60 mg, 0.33 mmol) and (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (100 mg, 0.18 mmol). (3-((4S)-6-(4-chlorophenyl)-4-(2-(ethylamino)-2-oxoethyl)-1-methyl-4H-benzo[f][1,2,4]triazolo[4,3-a][1,4]diazepin-8-yl)phenyl)boronic acid, BRD-N72 (7 mg, 7.7%) was isolated by preparative HPLC. ¹H-NMR (400 MHz, CD₃OD): δ 8.31 (brs, 1H), 8.12-8.09 (m, 1H), 8.07 (q, J=1.6 Hz, 1H), 7.91-7.88 (m, 1H), 7.83 (s, 1H), 7.67 (m, 2H), 7.61-7.58 (m, 2H), 7.50-7.43 (m, 3H), 4.72 (q, J=5.2 Hz, 1H), 3.50-3.42 (m, 1H), 3.32 (m, 2H), 3.02 (m, 1H), 2.73 (s, 3H), 1.22 (t, J=7.6 Hz, 3H). LRMS m/z: calcd for C₂₇H₂₅BClN₅O₃ [M+H]⁺: 514.2; found 514.2.

### 2-((4S)-6-(4-chlorophenyl)-1-methyl-8-phenyl-4H-benzo[f][1,2,4]triazolo[4,3-a][1,4]diazepin-4-yl)-N-ethylacetamide (BRD-E72c)

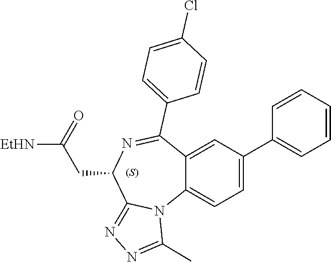

2-((4S)-6-(4-chlorophenyl)-1-methyl-8-phenyl-4H-benzo[1,2,4]triazolo[4,3-a][1,4]diazepin-4-yl)-N-ethylacetamide (BRD-N72c) was synthesized by following the procedure for the Suzuki coupling of phenylboronic acid (45 mg, 0.33 mmol) and (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (100 mg, 0.18 mmol). 2-((4S)-6-(4-chlorophenyl)-1-methyl-8-phenyl-4H-benzo[f][1,2,4]triazolo[4,3-a][1,4]diazepin-4-yl)-N-ethylacetamide, BRD-N72c (13 mg, 15%) was isolated by preparative HPLC. ¹H-NMR (400 MHz, CD₃OD): δ 8.13-8.08 (m, 1H), 7.92 (d, J=8.4 Hz, 1H), 7.69 (d, J=2.0 Hz, 1H), 7.65-7.60 (m, 4H), 7.51-7.43 (m, 5H), 4.74 (q, J=5.2 Hz, 1H), 3.50-3.42 (m, 1H), 3.33-3.31 (m, 3H), 2.76 (s, 3H), 1.21 (t, J=7.2 Hz, 3H). LRMS m/z: calcd for C₂₇H₂₄ClN₅O[M+H]⁺: 470.2; found 470.2.

### 2-((4S)-6-([1,1′-biphenyl]-4-yl)-1-methyl-8-phenyl-4H-benzo[f][1,2,4]triazolo[4,3-a][1,4]diazepin-4-yl)-N-ethylacetamide (BRD-E72s)

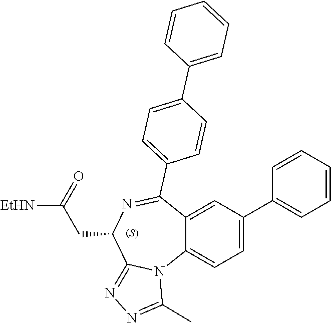

2-((4S)-6-([1,1′-biphenyl]-4-yl)-1-methyl-8-phenyl-4H-benzo[f][1,2,4]triazolo[4,3-a][1,4]diazepin-4-yl)-N-ethylacetamide, BRD-E72s (9.48 mg), was isolated as by-product in the synthesis of BRD-E72. ¹H-NMR (400 MHz, CD₃OD): δ 8.14 (m, 1H), 7.93 (d, J=8.4 Hz, 1H), 7.76 (m, 1H), 7.69-7.64 (m, 8H), 7.50-7.38 (m, 6H), 4.79 (q, J=5.2 Hz, 1H), 3.49 (m, 1H), 3.36-3.31 (m, 3H), 2.79 (s, 3H), 1.23 (t, J=7.2 Hz, 3H). LRMS m/z: calcd for C₃₃H₂₉ClN₅O[M+H]⁺: 512.2; found 512.2.

### Synthesis of BRD-E75 and BRD-E75C

BRD-E75 and BRD-E75c were synthesized using processes disclosed in WO2015081280 to Arnold et al., which is hereby incorporated by reference in its entirety.

### General Procedure for Buchwald Coupling of Thiophenols

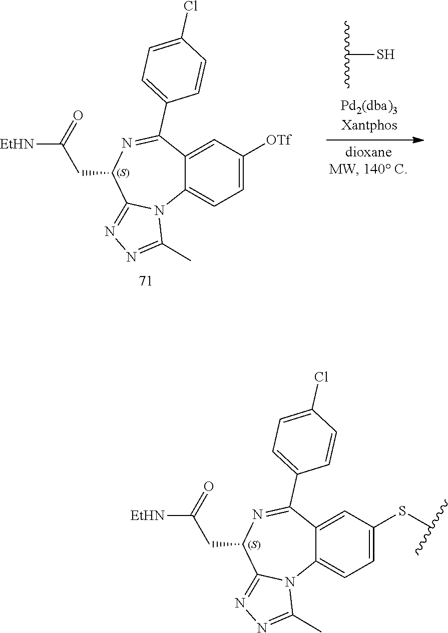

To an 8 mL microwave reaction vial containing a solution of (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (1 eq) in 1,4-dioxane (3 mL) was added the thiol derivative (1.8 eq) and DIPEA (2 eq). The resulting solution was purged with nitrogen for 10 min, at which point Xantphos (2 eq) and Pd₂(dba)₃ (0.1 equiv.) were added and the resulting mixture was heated at 140° C. under microwave irradiation for 30 min, then cooled to ambient temperature and concentrated under reduced pressure. The resulting mixture was purified by preparative HPLC [column: X-Select C18 (19×150 mm, 5 μm); mobile phase A: 0.1% formic acid in water; mobile phase B: ACN; flowrate: 15 mL/min]. Fractions containing the product were combined and lyophilized.

### (4-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)thio)phenyl)boronic acid (BRD-E75)

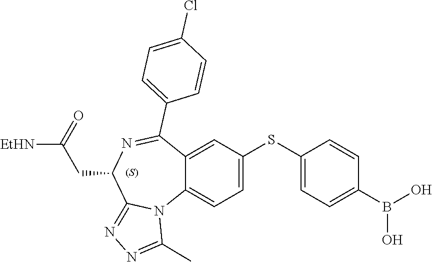

(4-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)thio)phenyl)boronic acid (BRD-E75) was synthesized by following the procedure for the Buchwald coupling of 4-mercaptophenylboronic acid (57 mg, 0.33 mmol) and (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (100 mg, 0.18 mmol). (4-(((4S)-6-(4-chlorophenyl)-4-(2-(ethylamino)-2-oxoethyl)-1-methyl-4H-benzo[f][1,2,4]triazolo[4,3-a][1,4]diazepin-8-yl)thio)phenyl)boronic acid, BRD-E75 (20 mg, 20%) was isolated by preparative HPLC. ¹H-NMR (400 MHz, CD₃OD): δ 8.36 (brs, 1H), 7.75-7.70 (m, 3H), 7.62 (d, J=8.0 Hz, 2H), 7.47-7.34 (m, 7H), 7.02 (s, 1H), 4.64 (q, J=5.2 Hz, 1H), 3.37-3.33 (m, 1H), 3.29-3.27 (m, 3H), 2.67 (s, 3H), 1.19 (t, J=7.6 Hz, 3H). LRMS m/z: calcd for C₂₇H₂₅BClN₅O₃S [M+H]⁺: 546.2; found 546.0.

### 2-((4S)-6-(4-chlorophenyl)-1-methyl-8-(phenylthio)-4H-benzo[f][1,2,4]triazolo[4,3-a][1,4]diazepin-4-yl)-N-ethylacetamide (BRD-E75c)

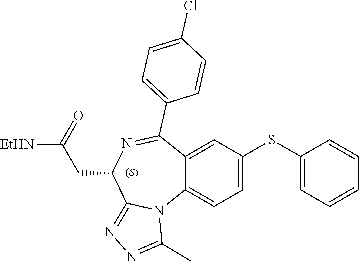

2-((4S)-6-(4-chlorophenyl)-1-methyl-8-(phenylthio)-4H-benzo[f][1,2,4]triazolo[4,3-a][1,4]diazepin-4-yl)-N-ethylacetamide (BRD-E75c) was synthesized by following the procedure for the Buchwald coupling of thiophenol (66 mg, 0.30 mmol) and (4S)-6-(4-chlorophenyl)-4-(2-(ethylamino)-2-oxoethyl)-1-methyl-4H-benzo[f][1,2,4]triazolo[4,3-a][1,4]diazepin-8-yl trifluoromethanesulfonate, 71 (100 mg, 0.18 mmol). 2-((4S)-6-(4-chlorophenyl)-1-methyl-8-(phenylthio)-4H-benzo[/][1,2,4]triazolo[4,3-a][1,4]diazepin-4-yl)-N-ethylacetamide, BRD-E75c (14 mg, 18.9%) was isolated by preparative HPLC. ¹H-NMR (400 MHz, CD₃OD): δ 8.36 (brs, 1H), 7.73-7.71 (m, 1H), 7.66-7.64 (m, 1H), 7.51-7.48 (m, 2H), 7.41-7.37 (m, 7H), 7.03 (s, 1H), 4.64 (q, J=5.2 Hz, 1H), 3.37-3.33 (m, 1H), 3.28-3.25 (m, 3H), 2.05 (s, 3H), 1.19 (t, J=7.2 Hz, 3H). LRMS m/z: calcd for C₂₇H₂₄ClN₅OS [M+H]⁺: 502.1; found 502.2.

### Example 2—CURE-PRO-Mediated BRD4 Degradation

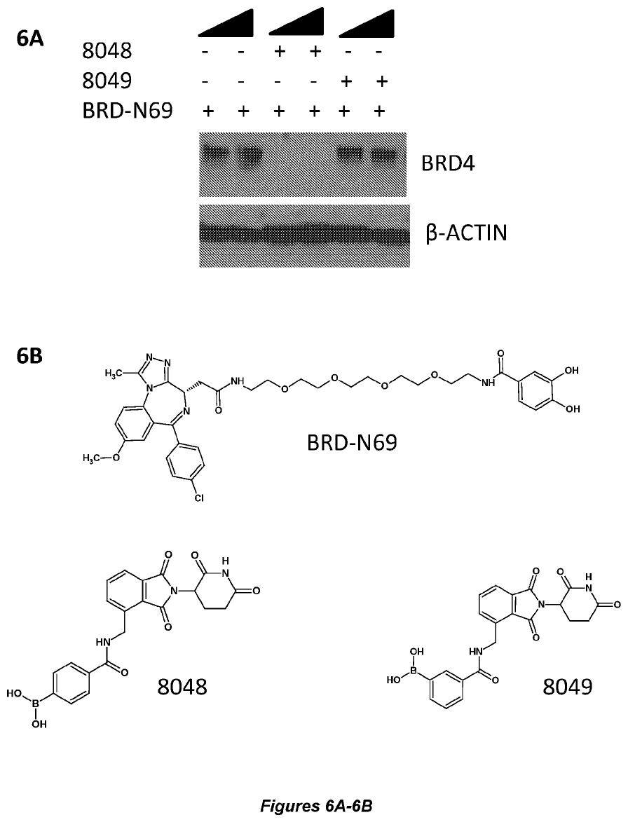

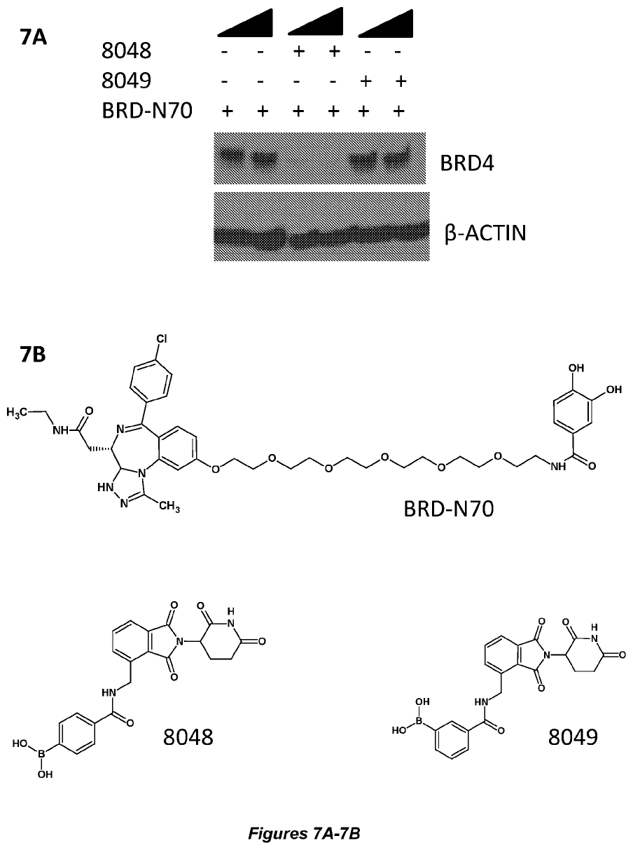

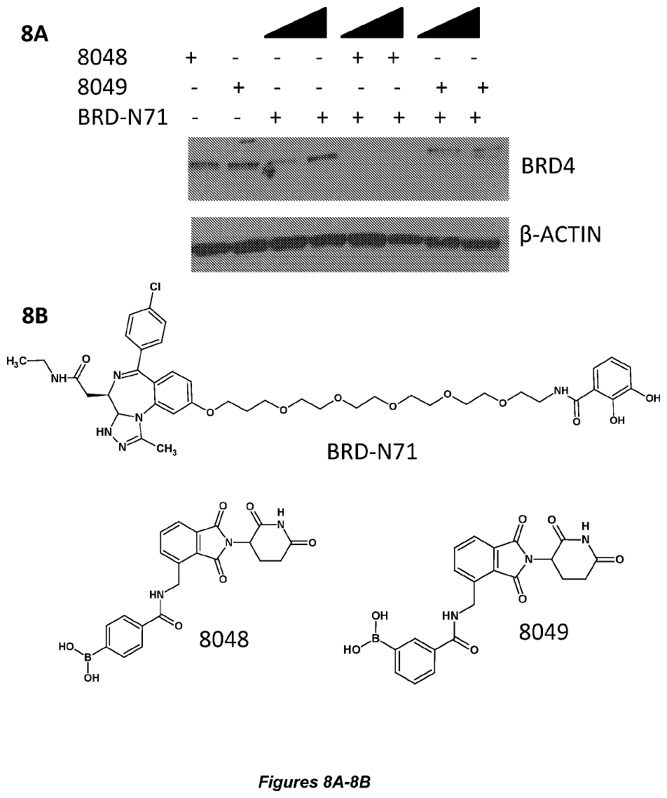

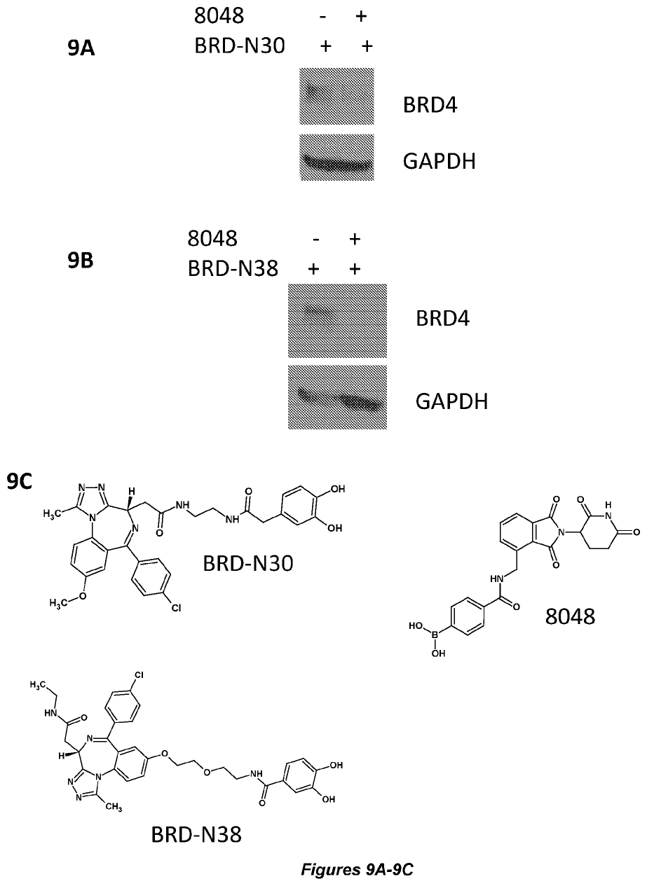

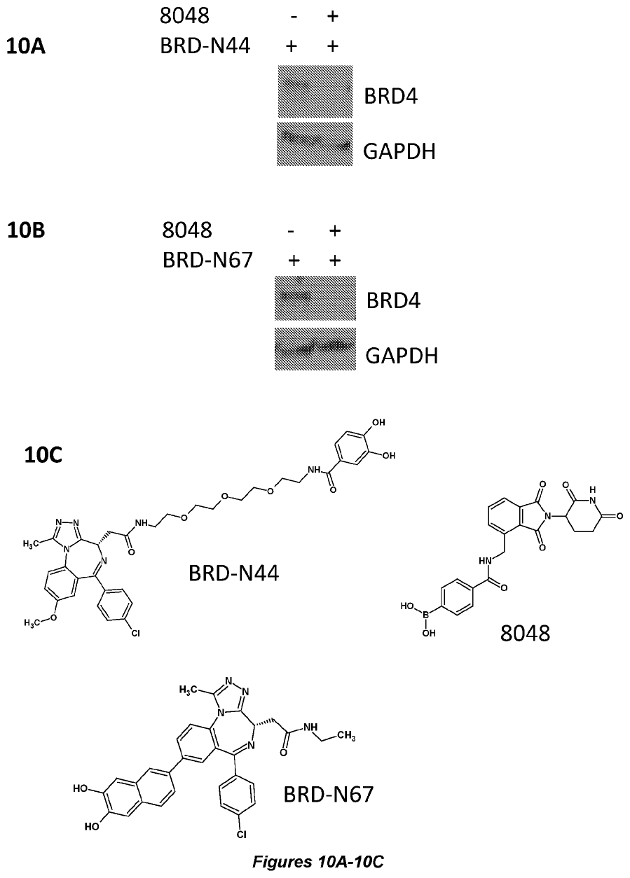

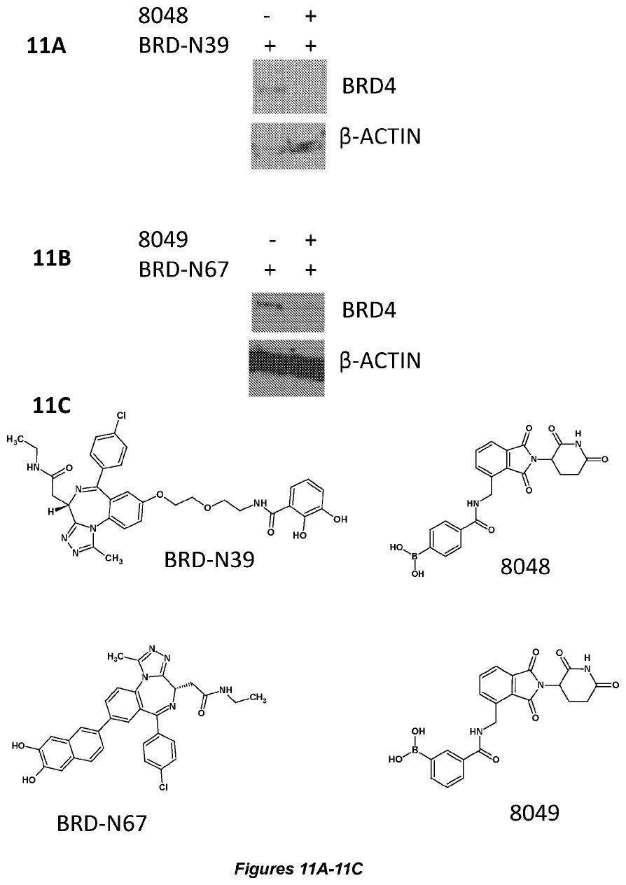

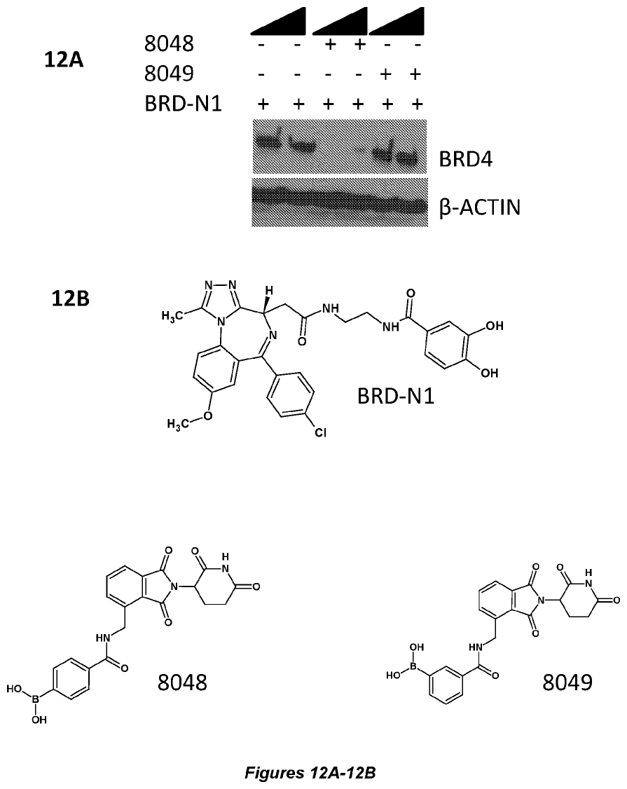

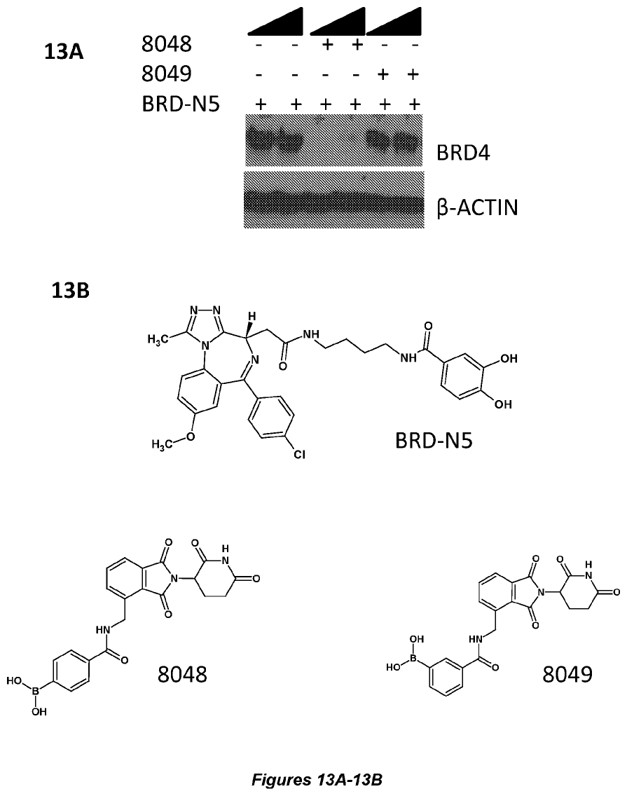

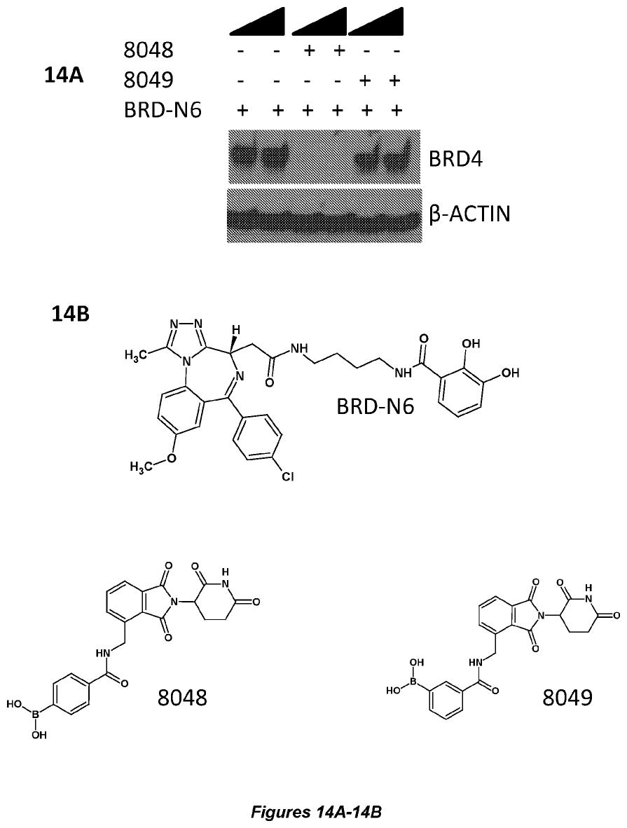

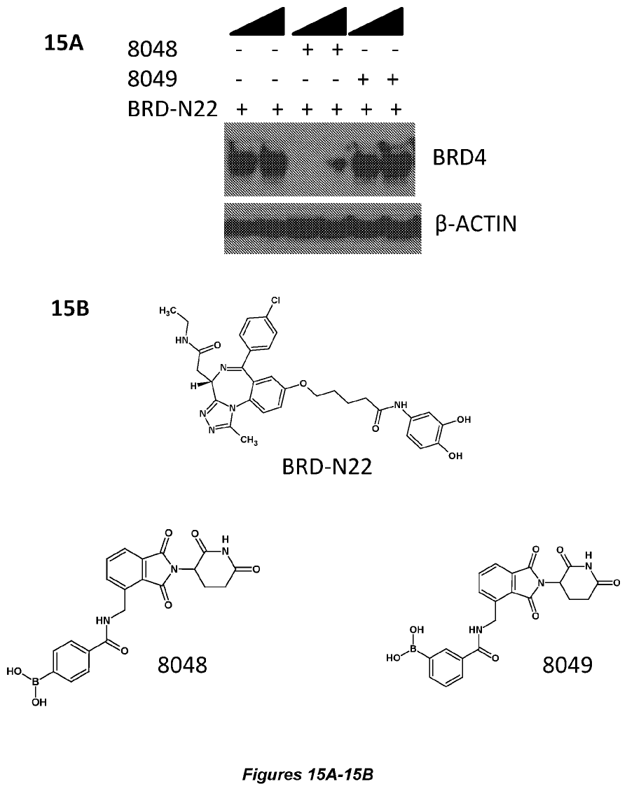

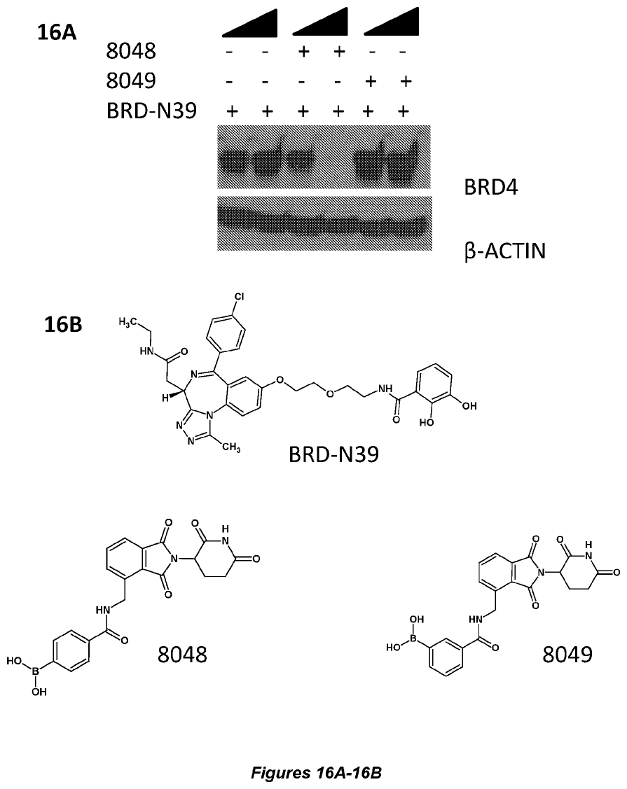

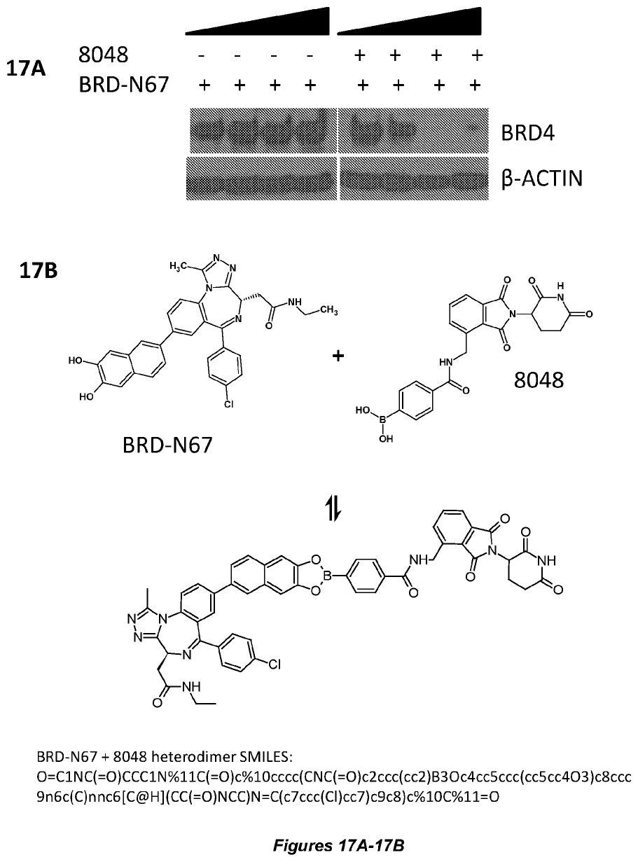

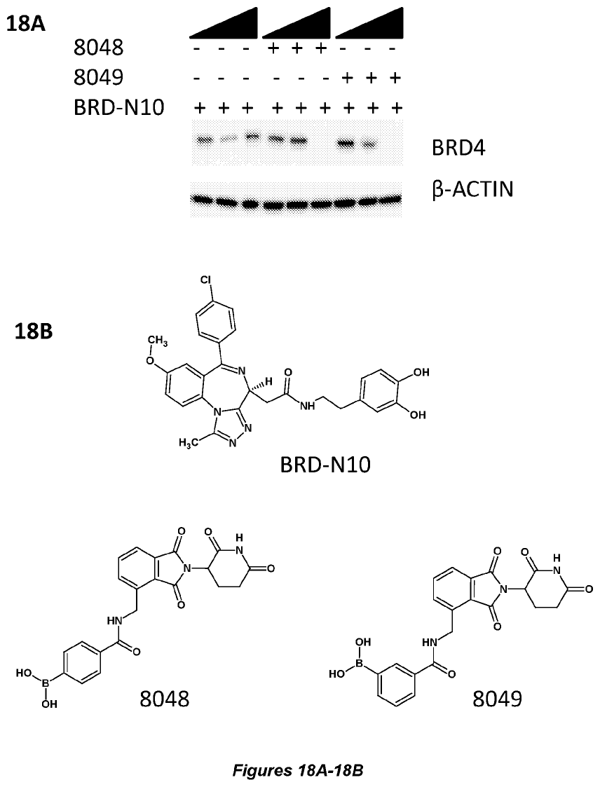

HeLa cells (2.5×106) were treated for 24 hours with the sample compounds solubilized in DMSO. Compounds were added at 1-10 μM each. Standardized protein samples were electrophoresed and imaged as described in the immunoblotting description above. FIGS. 6-18 depicts the CURE-PRO-mediated BRD4 degradation for BRD monomers (JQ1 derivatives comprising of catechol linkers), and CRBN ligands, 8048 and 8049 (pomalidomide derivatives comprising boronic acid linkers) combined in a 1:1 ratio. In the presence of only the BRD4 monomers (FIG. 6: BRD-N69, FIG. 7: BRD-N69; FIG. 8: BRD-N71; FIG. 9: BRD-N30 and BRD-N38; FIG. 10: BRD-N44 and BRD-N67; FIG. 11: BRD-N39 and BRD-N67; FIG. 12: BRD-N1; FIG. 13: BRD-N5; FIG. 14: BRD-N6; FIG. 15: BRD-N22; FIG. 16: BRD-N39; FIG. 17: BRD-N67; FIG. 18: BRD-N10) there is no degradation of BRD4. Co-treatment of BRD-N69, BRD-N70, BRD-N71, BRD-N30, BRD-N38, BRD-N44, BRD-N67, BRD-N39, BRD-N1, BRD-N5, BRD-N6, BRD-N10 or BRD-N22 and 8048, causes degradation of BRD4. Co-treatment of BRD-N10 (FIG. 18) or BRD-N67 (FIG. 11) and 8049 causes degradation of BRD4. There is no evidence of BRD4 degradation with co-treatment of BRD-N69, BRD-N70, BRD-N71, BRD-N1, BRD-N5, BRD-N6, BRD-N10, BRD-N39 or BRD-N22 and 8049 (FIG. 6-8, 12-6).

### Example 3—Dose Response of CURE-PRO-Mediated BRD Degradation

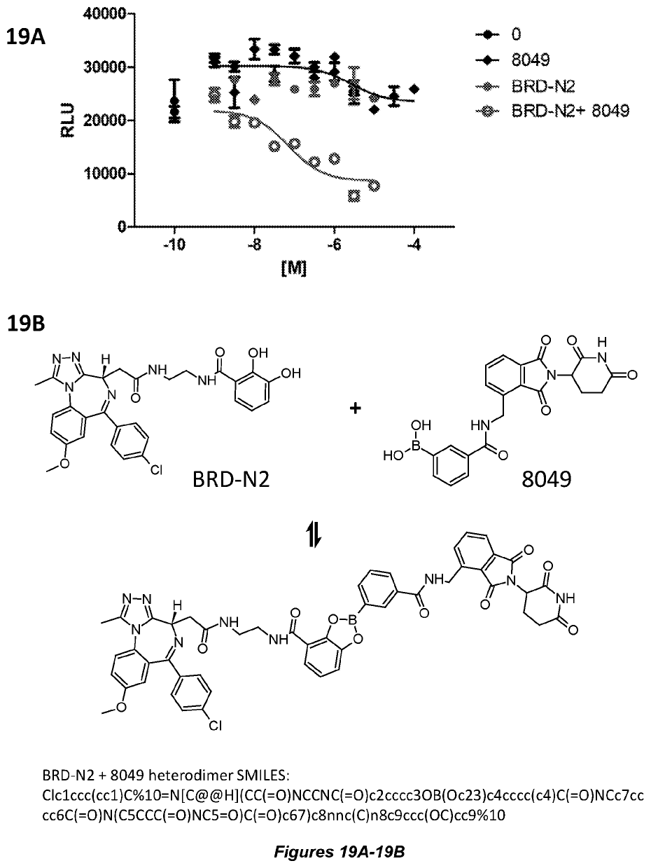

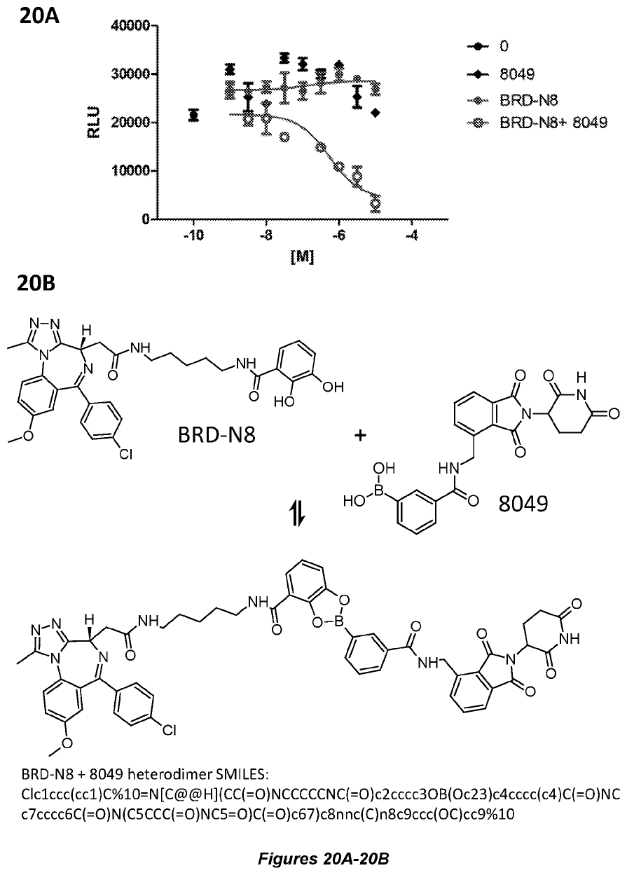

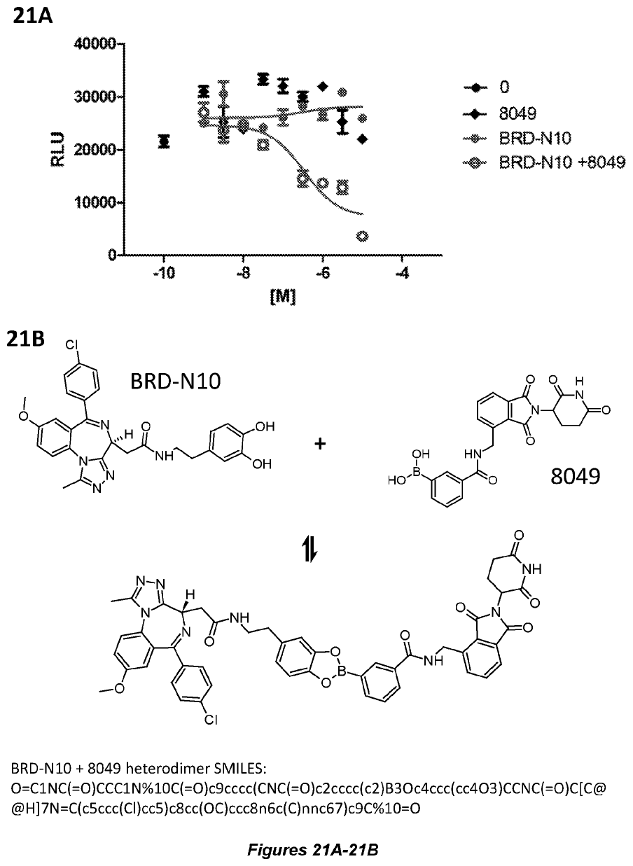

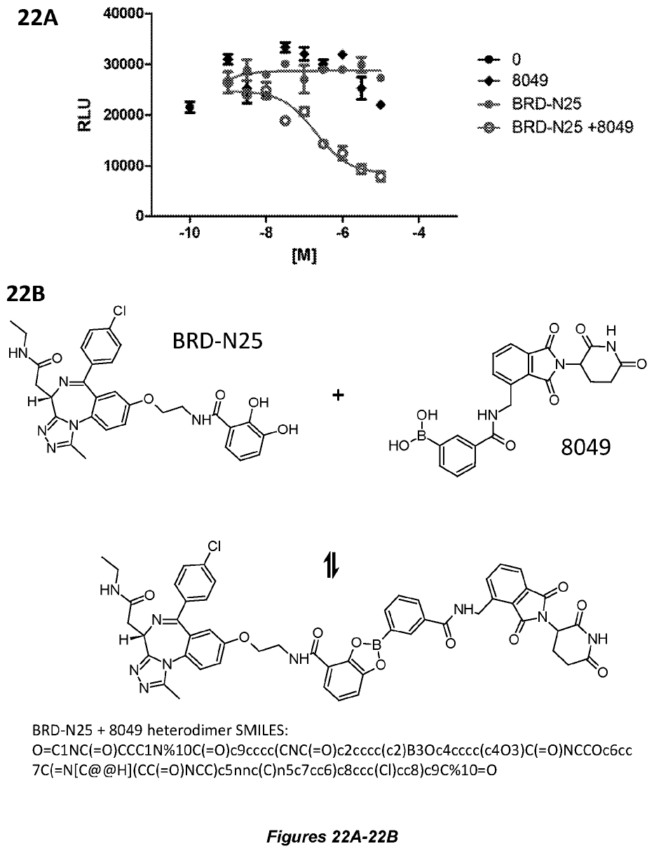

MV-4-11 cells (5×104) were treated for 24 hours with the sample compounds solubilized in DMSO. The compounds were added at 1 nM-100 μM each. Cellular viability was determined using the CellTiter-Glo® Luminescent Cell Viability Assay (Promega) described above. FIGS. 19-22 depict the CURE-PRO-mediated loss of cellular viability for BRD monomers (JQ1 derivatives comprising of catechol linkers), and CRBN ligand, 8049 (pomalidomide derivatives comprising boronic acid linkers) combined in a 1:1 ratio. In the presence of only the BRD4 or 8049 monomers, little loss of viability was observed. When BRD-N2 (FIG. 19), BRD-N8 (FIG. 20), BRD-N10 (FIG. 21), or BRD-N25 (FIG. 22), were co-dosed with 8049 a concentration-dependent loss in cellular viability was observed.

### Example 4—CURE-PRO-Mediated BRD4 Degradation

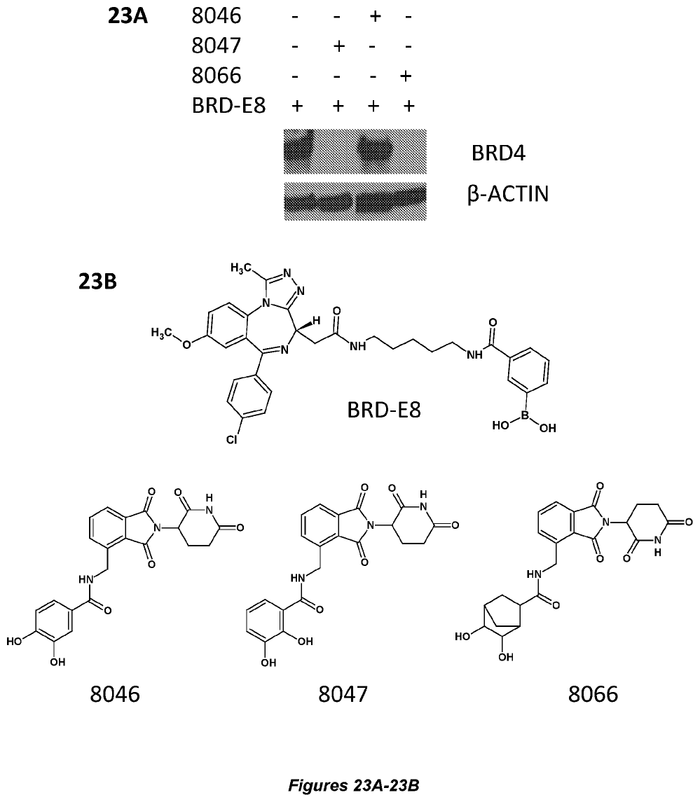

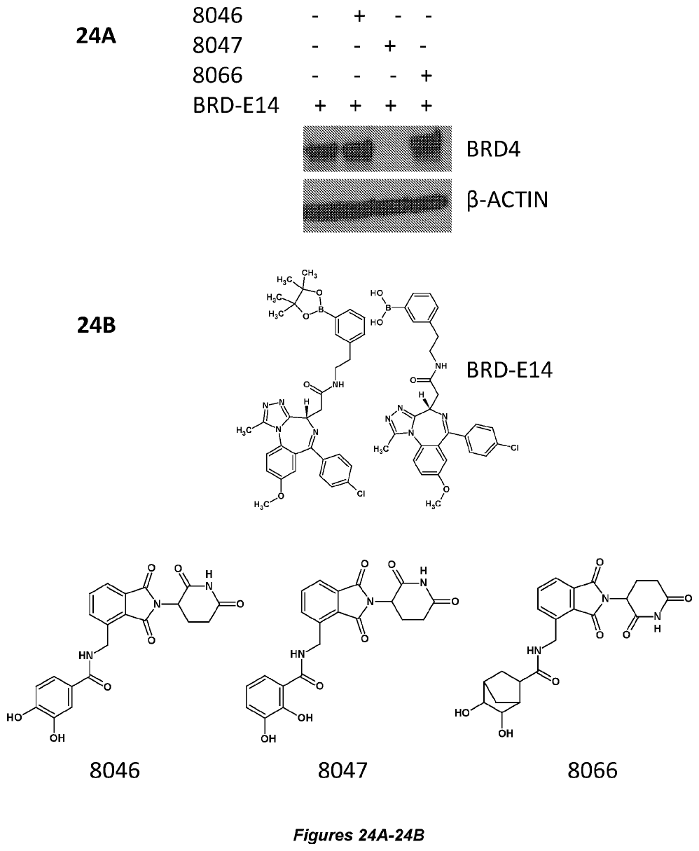

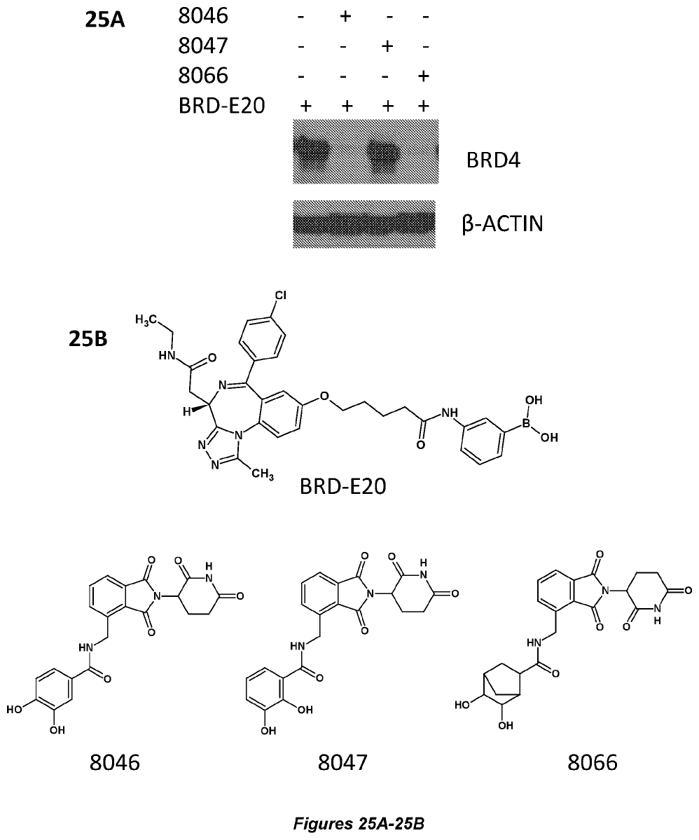

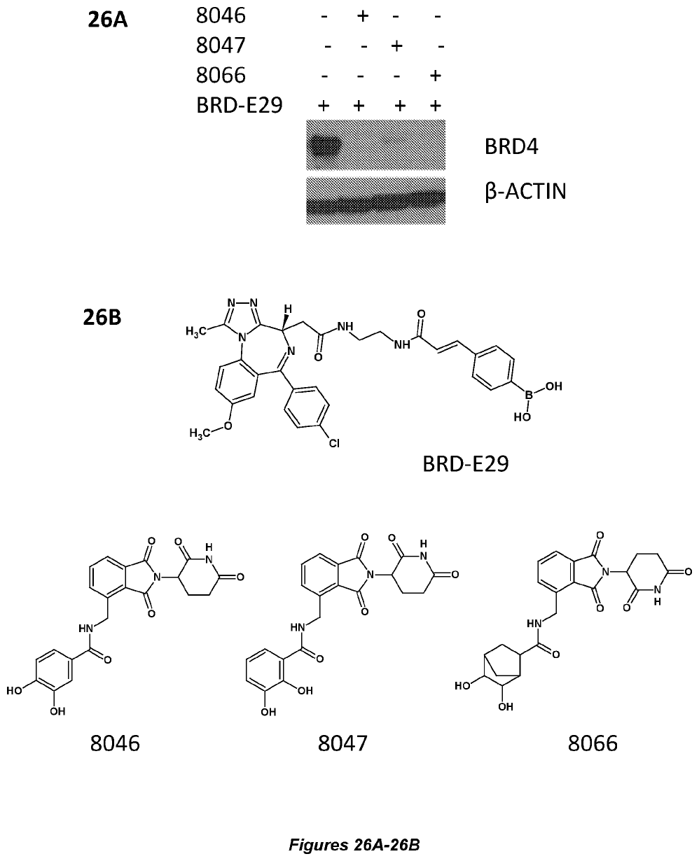

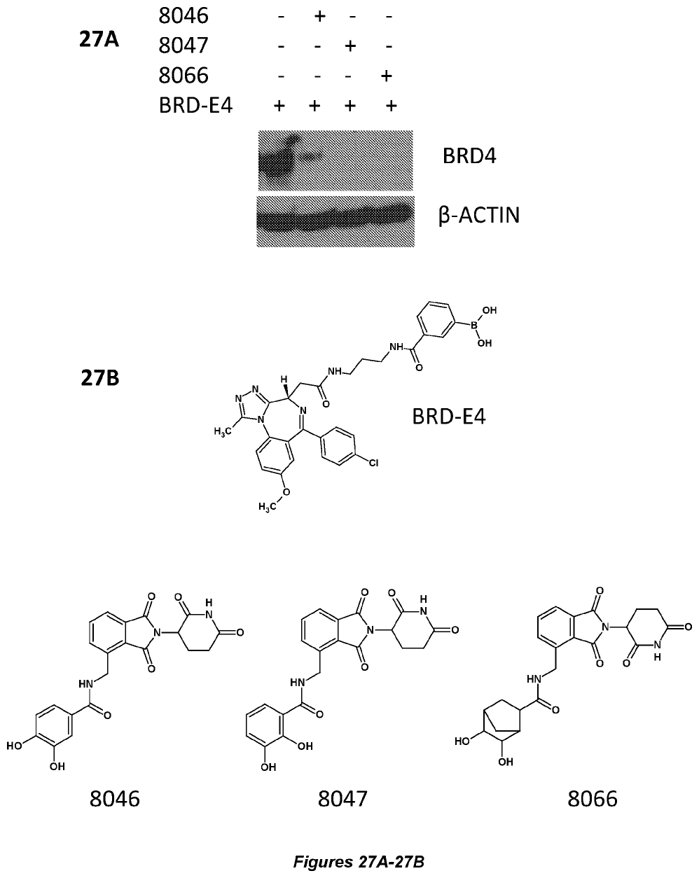

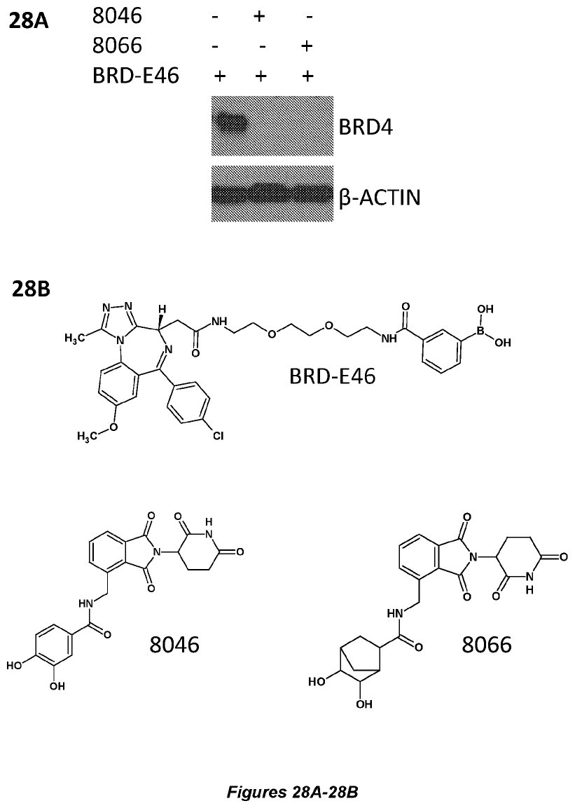

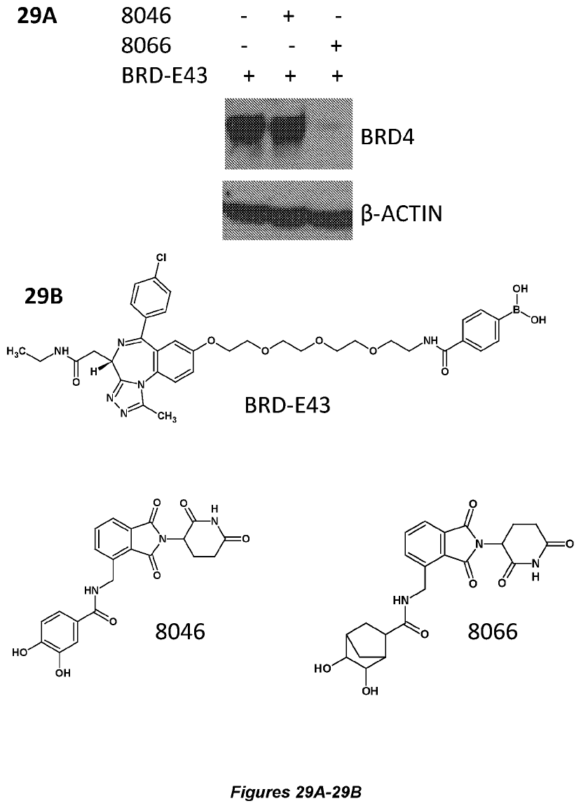

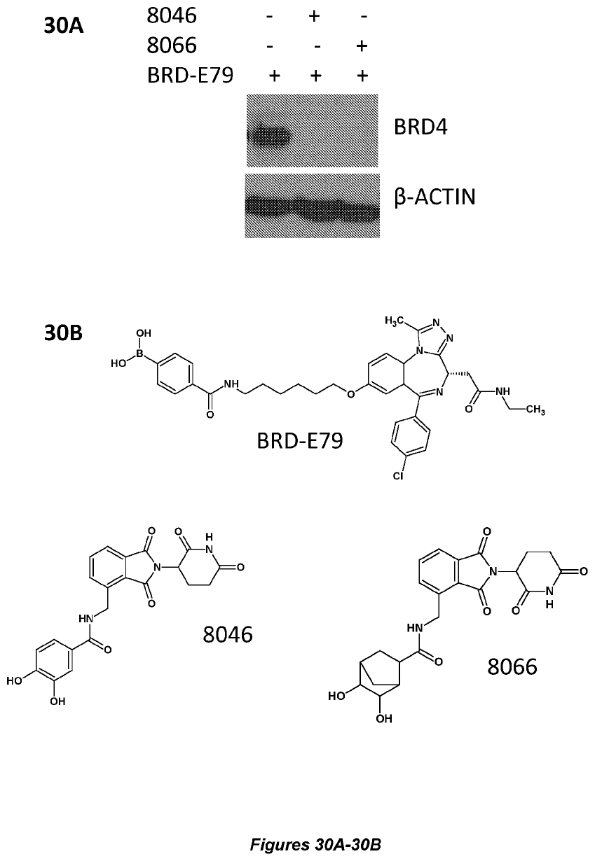

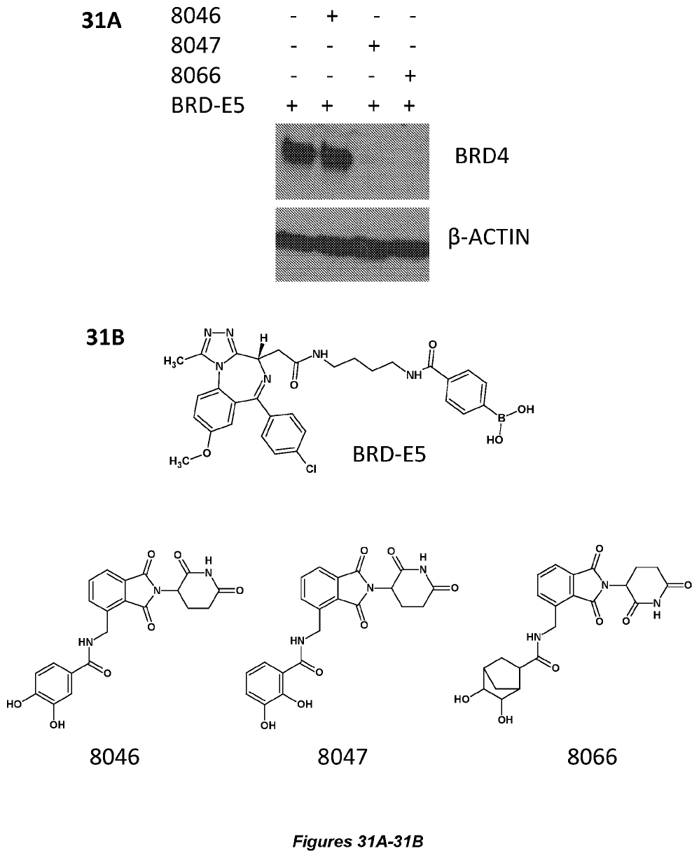

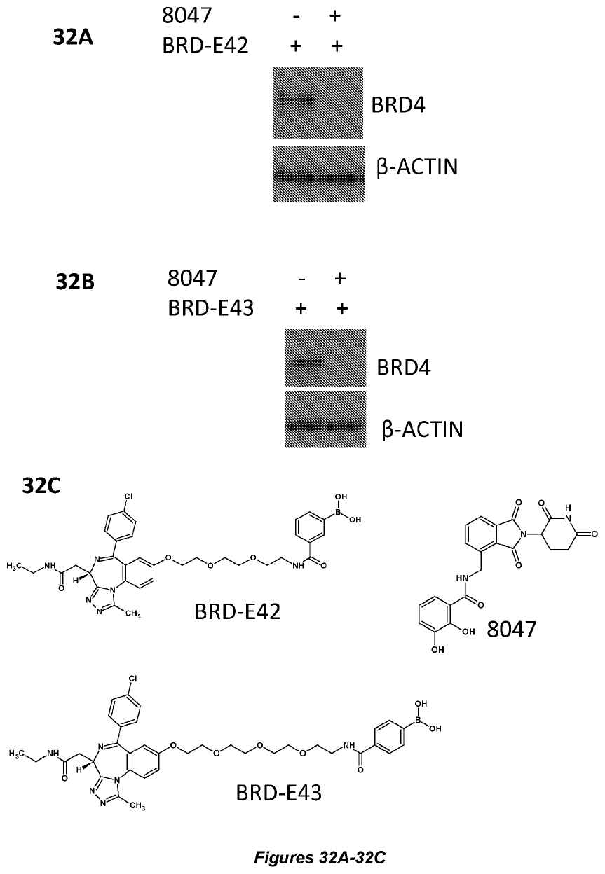

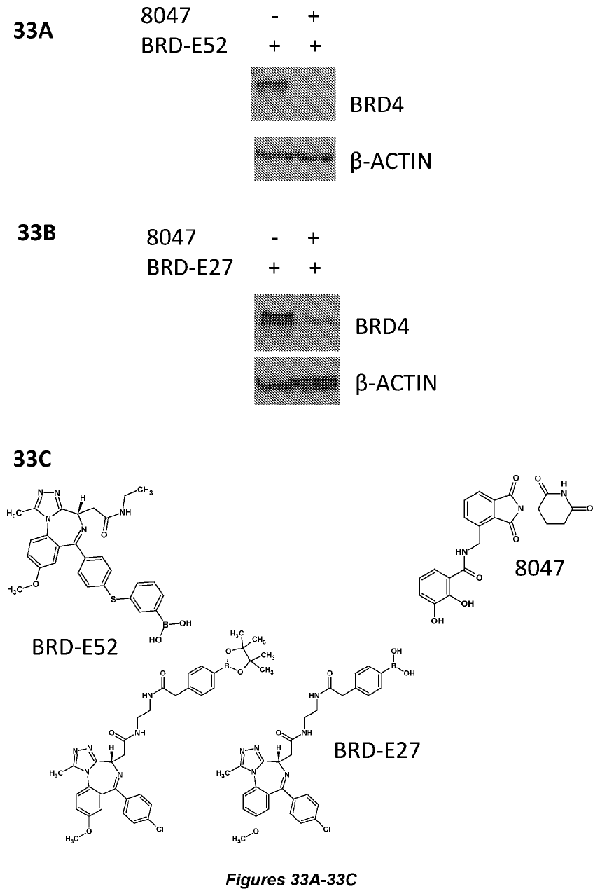

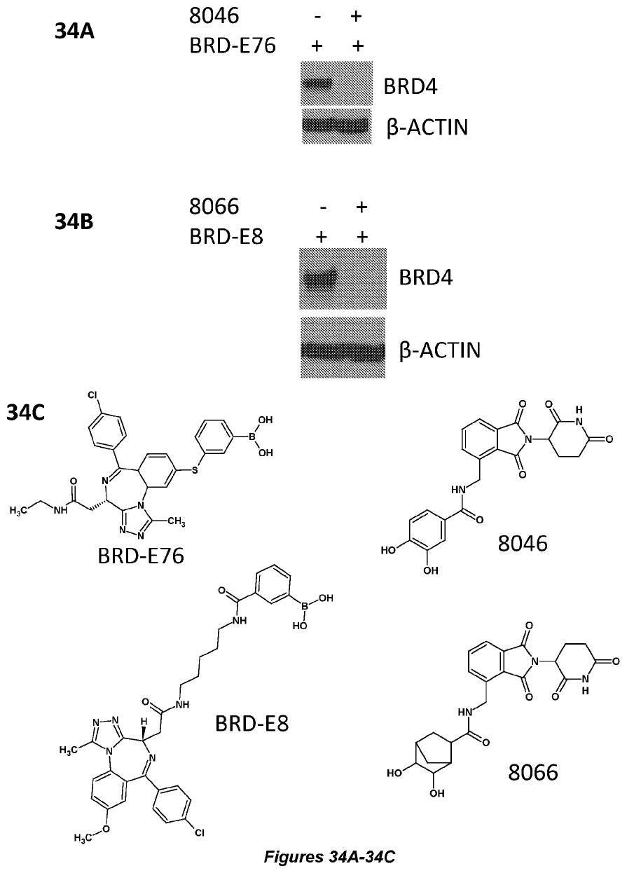

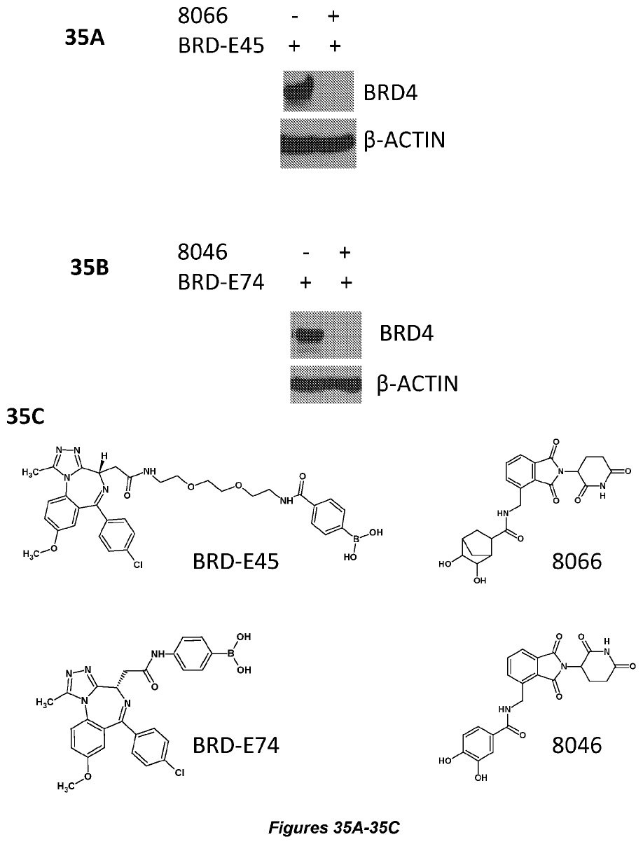

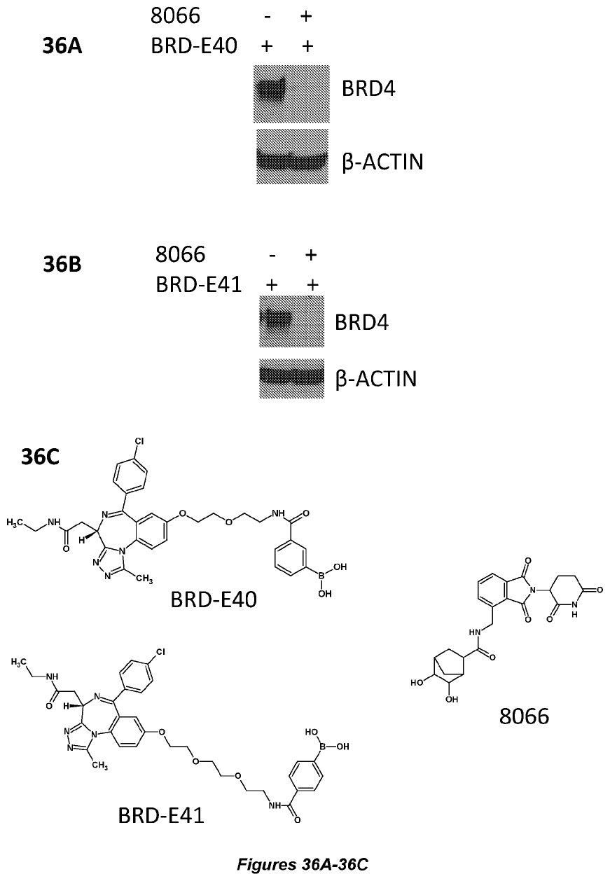

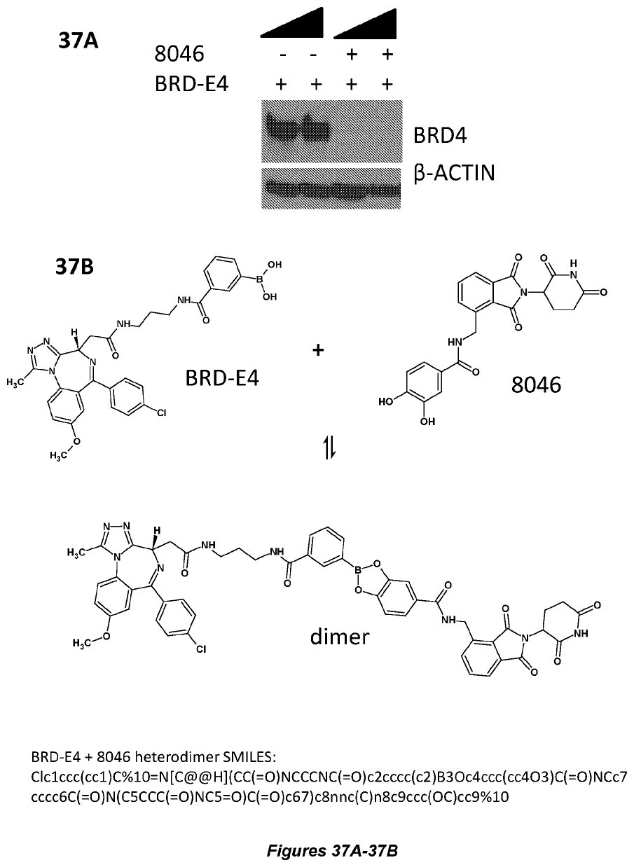

HeLa cells (2.5×106) were treated for 24 hours with the compounds solubilized in DMSO. The compounds were added at 10 μM each. Standardized protein samples were electrophoresed and imaged as described in the immunoblotting section above. FIGS. 23-27 depicts the CURE-PRO-mediated BRD4 degradation for BRD monomers (JQ1 derivatives comprising of boronic acid linkers), and CRBN ligands, 8046, 8047 and 8066 (pomalidomide derivatives comprising diol linkers) combined in a 1:1 ratio. In the presence of only the indicated BRD4 monomers (FIGS. 23-27: lane 1) there is no degradation of BRD4. Co-treatment of BRD-E8 (FIG. 23), BRD-E20 (FIG. 25), BRD-E29 (FIG. 26), BRD-E4 (FIGS. 27 and 37 (lanes 3 and 4)), BRD-E20 (FIG. 25), BRD-E46 (FIG. 28), BRD-E20 (FIG. 25), BRD-E79 (FIG. 30), BRD-E20 (FIG. 25), BRD-E76 (FIG. 34A), and BRD-E74 (FIG. 35B) together with 8046 (lane 2, unless otherwise indicated), causes degradation of BRD4. Co-treatment of BRD-E14 (FIG. 24), BRD-E29 (FIG. 26), BRD-E4 (FIG. 27), BRD-E5 (FIG. 31), BRD-E42 (FIG. 32A, lane 2), BRD-E43 (FIG. 32B, lane 2), BRD-E52 (FIG. 33A, lane 2), BRD-E27 (FIG. 33B, lane 2), BRD-E76 (FIG. 34A), and BRD-E74 (FIG. 35B) together with 8047 (lane 3, unless otherwise indicated), causes degradation of BRD4. Co-treatment of BRD-E8 (FIG. 23), BRD-E20 (FIG. 25), BRD-E29 (FIG. 26), BRD-E4 (FIG. 27), BRD-E46 (FIG. 28, lane 3), BRD-E43 (FIG. 29, lane 3), BRD-E79 (FIG. 29, lane 3), BRD-E5 (FIG. 31), BRD-E8 (FIG. 34B, lane 2), BRD-E45 (FIG. 35A, lane 2), BRD-E40 (FIG. 36A, lane 2), and BRD-E41 (FIG. 36B, lane 2), together with 8066 (lane 4, unless otherwise indicated), causes degradation of BRD4.

### Example 5—Concentration Dependence of CURE-PRO-Mediated BRD4 Degradation

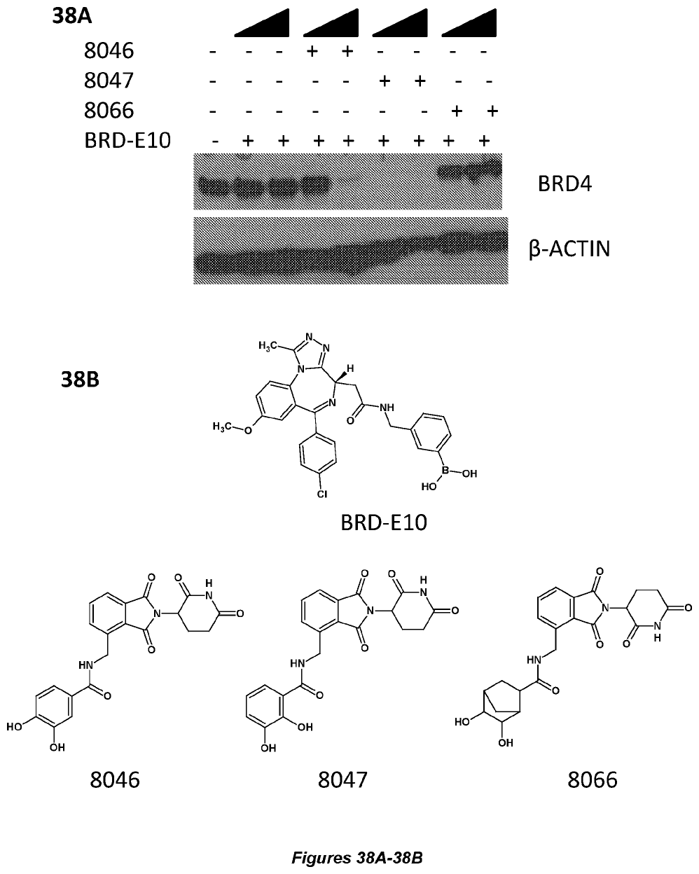

HeLa cells (2.5×10⁶) were treated for 24 hours with the compounds solubilized in DMSO. Compounds were added at 10 μM and 100 μM each. Standardized protein samples were electrophoresed and imaged as described in the immunoblotting section above. FIG. 38 depicts the CURE-PRO-mediated BRD4 degradation for the BRD-E10 monomer (a JQ1 derivative comprising of boronic acid linkers), and CRBN ligands, 8046 and 8047 (pomalidomide derivatives comprising diol linkers) combined in a 1:1 ratio was observed, but not with 8066.8047 and BRD-E10 demonstrated complete degradation at 10 μM, whereas 8046 and BRD-E10 only demonstrated BRD4 degradation at 100 μM.

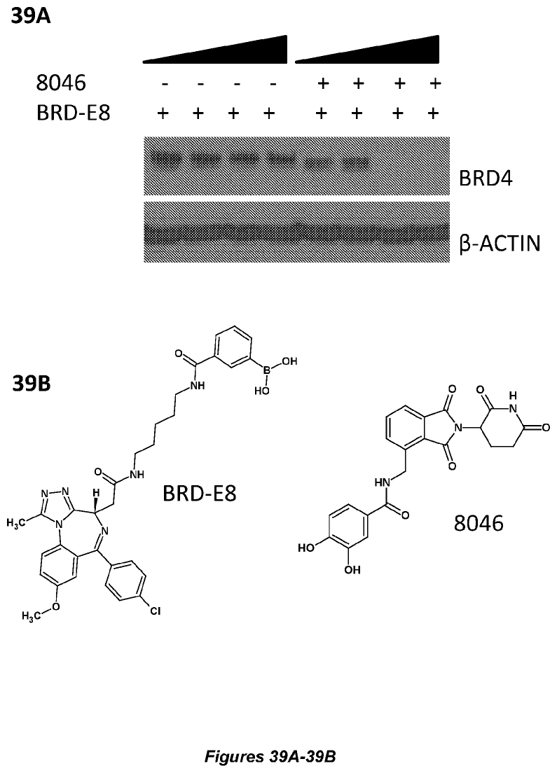

HeLa cells (2.5×10⁶) were treated for 24 hours with the compounds solubilized in DMSO. Compounds were added at 1 nM-1 μM each. Standardized protein samples were electrophoresed and imaged as described in the immunoblotting section above. FIG. 39 depicts the concentration-dependent CURE-PRO-mediated BRD4 degradation for BRD-E8 monomer (JQ1 derivatives comprising of boronic acid linkers), and the CRBN ligand, 8046 (a pomalidomide derivatives comprising diol linkers) combined in a 1:1 ratio. Degradation is observed at 100 nM and 1 μM when BRD-E8 is co-dosed with 8046, but no change in expression is observed when cells are treated with BRD-E8 alone.

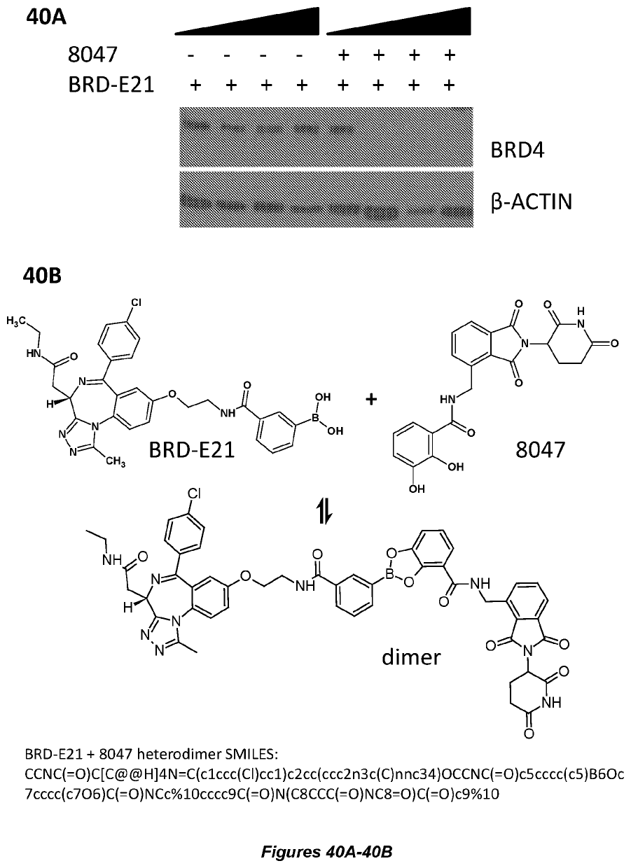

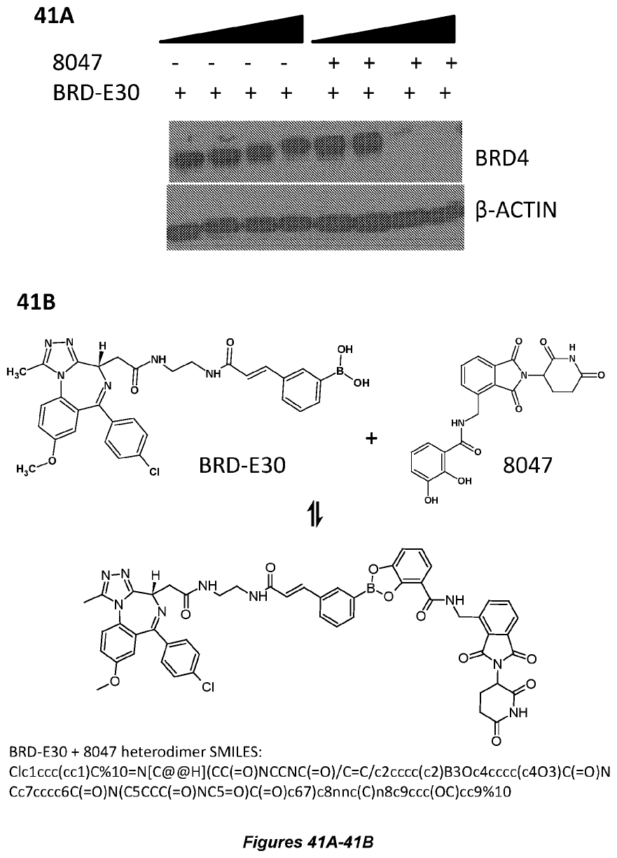

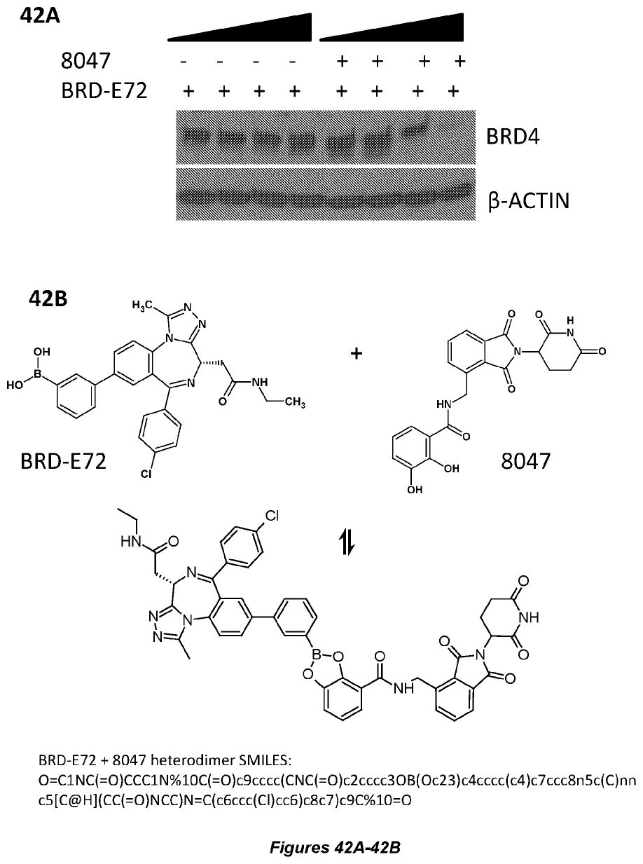

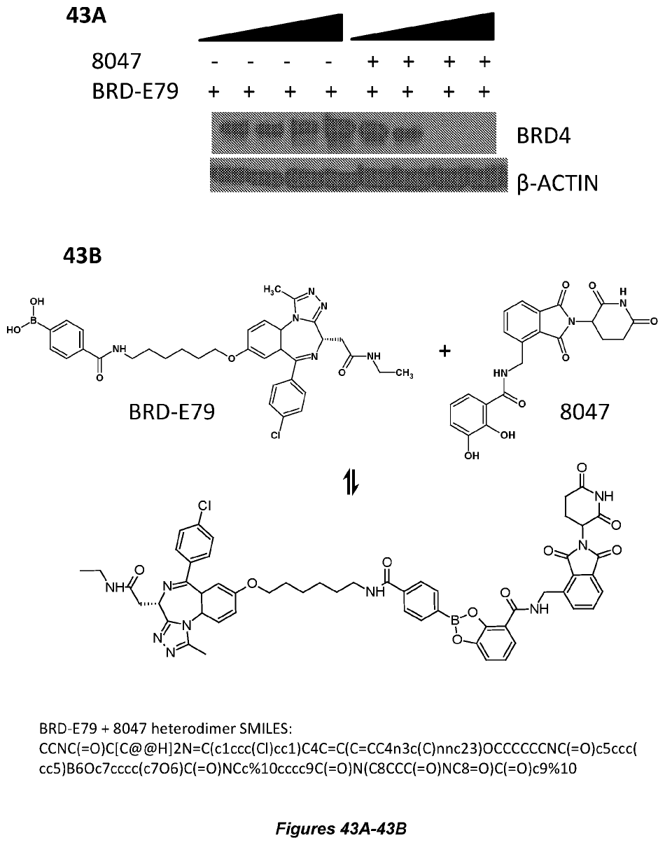

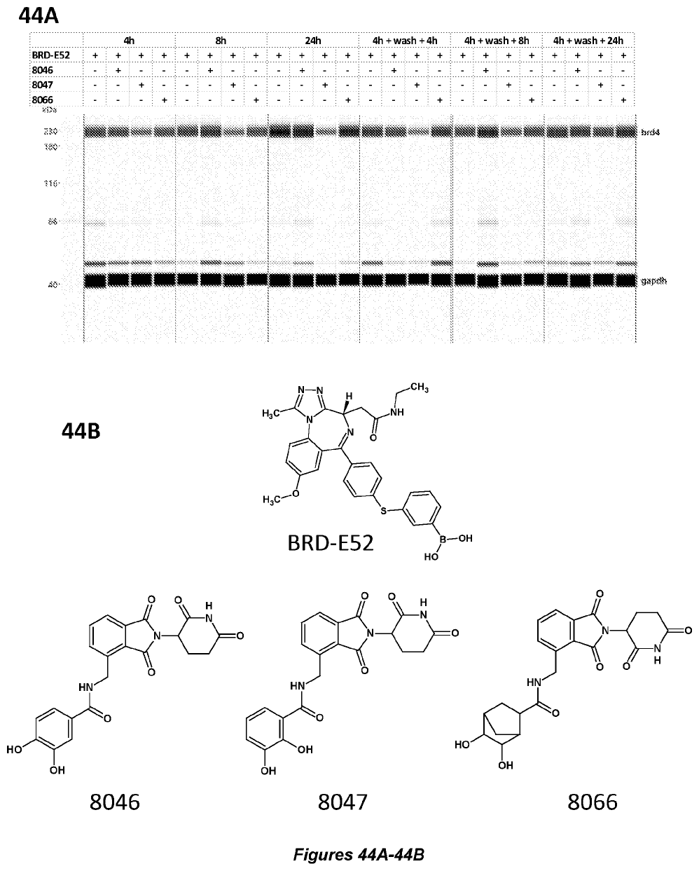

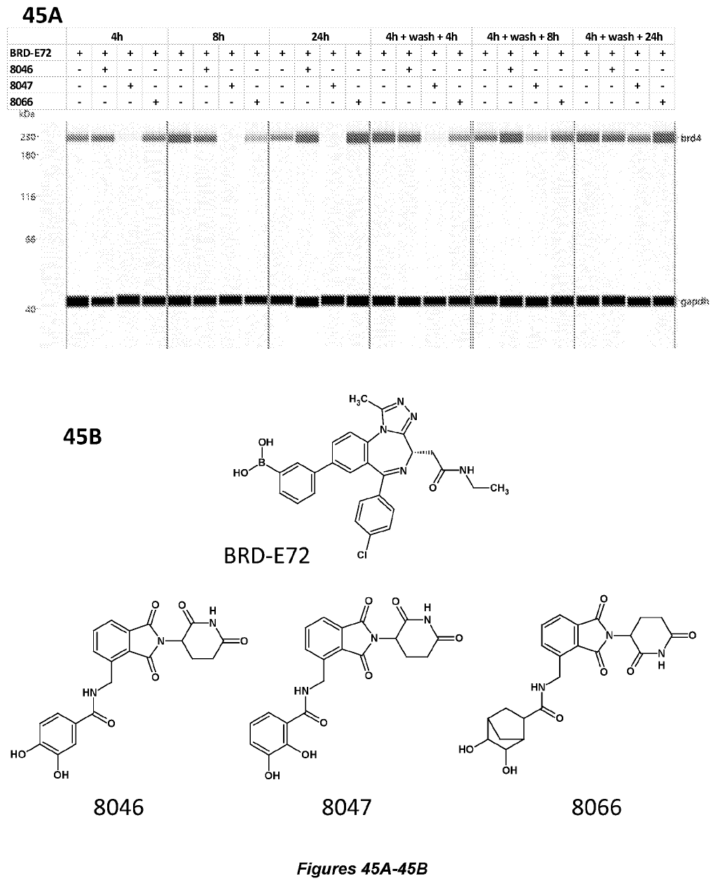

HeLa cells (2.5×10⁶) were treated for 24 hours with the compounds solubilized in DMSO. Compounds were added at 1 nM-1 μM each. Standardized protein samples were electrophoresed and imaged as described in the immunoblotting section above. FIGS. 40-43 depicts the concentration-dependent CURE-PRO-mediated BRD4 degradation for the BRD-E21 (FIG. 40), BRD-E30 (FIG. 41), BRD-E72 (FIG. 42), and BRD-E79 (FIG. 43) monomers, (JQ1 derivatives comprising of boronic acid linkers), and the CRBN ligand, 8047 (a pomalidomide derivative comprising diol linkers) combined in a 1:1 ratio. Degradation is observed from 10 nM when BRD-E21 is co-dosed with 8047 (FIG. 40, lane 6), but no change in expression is observed when cells are treated with BRD-E21 alone. Degradation is observed from 100 nM for BRD-E30 (FIG. 41, lane7), BRD-E72 (FIG. 42, lane7), and BRD-E79 (FIG. 43, lane7) is co-dosed with 8047, but no change in expression is observed when cells are treated with the BRD monomers alone (FIGS. 40-43, lanes 1-4).

### Example 6—Time Trials of CURE-PRO Exposure

HeLa cells (2.5×10⁶) were treated for 4-24 hours with the compounds solubilized in DMSO. After 4 hours, indicated points were washed and cells were left in full growth media. Compounds were added at 10 μM each. Standardized protein samples were electrophoresed and imaged as described in the WES ProteinSimple section above. FIGS. 44 and 45 depict the time-dependence of CURE-PRO-mediated BRD4 degradation with the BRD-E52 ligand (FIG. 44) and BRD-E72 ligand (FIG. 45), both JQ1 derivatives comprising of boronic acid linkers, and the CRBN binding ligands (8046, 8047, and 8066), pomalidomide derivatives comprising diol linkers. Co-dosing with CRBN ligand 8047 demonstrates marked BRD4 degradation after 4h with sustained degradation for up to 8h after drugs are washed out.

### Example 7—Concentration Dependence of CURE-PRO-Mediated BRD4 Degradation

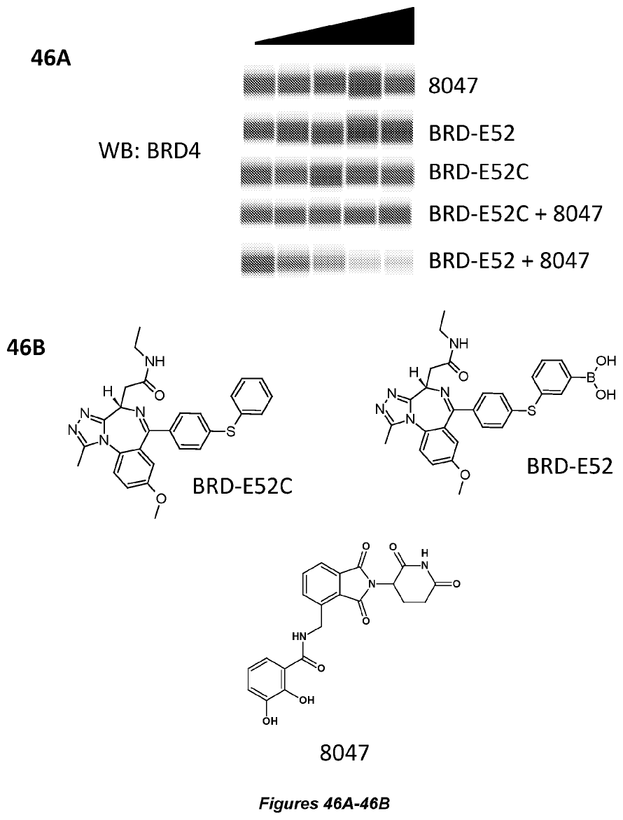

HeLa cells (2.5×10⁶) were treated for 24 hours with the compounds solubilized in DMSO. Compounds were added at 100 nM-10 μM each. Standardized protein samples were electrophoresed and imaged as described in the WES ProteinSimple section above. FIG. 46 depicts the concentration-dependence of CURE-PRO-mediated BRD4 degradation and the requirement for monomer dimerization. The BRD-E52 monomer and the CRBN binding ligand 8047, caused a decrease in BRD4 protein expression from 300 nM, but the control compound, BRD-E52C, that is incapable of forming a self-assembled dimer with 8047, failed to induce degradation even at 10 μM.

### Example 8—CURE-PRO Concentration Dependence of Cellular Viability

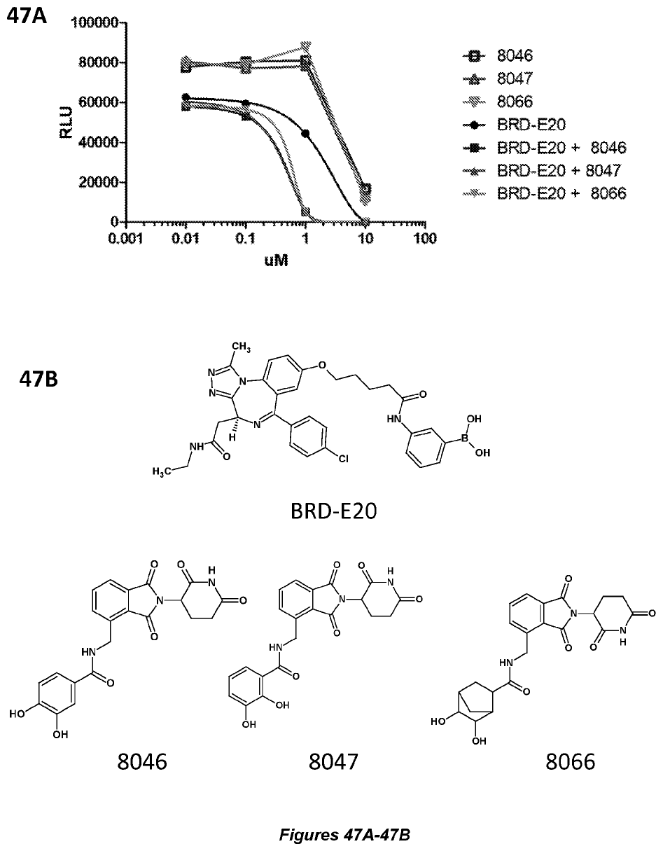

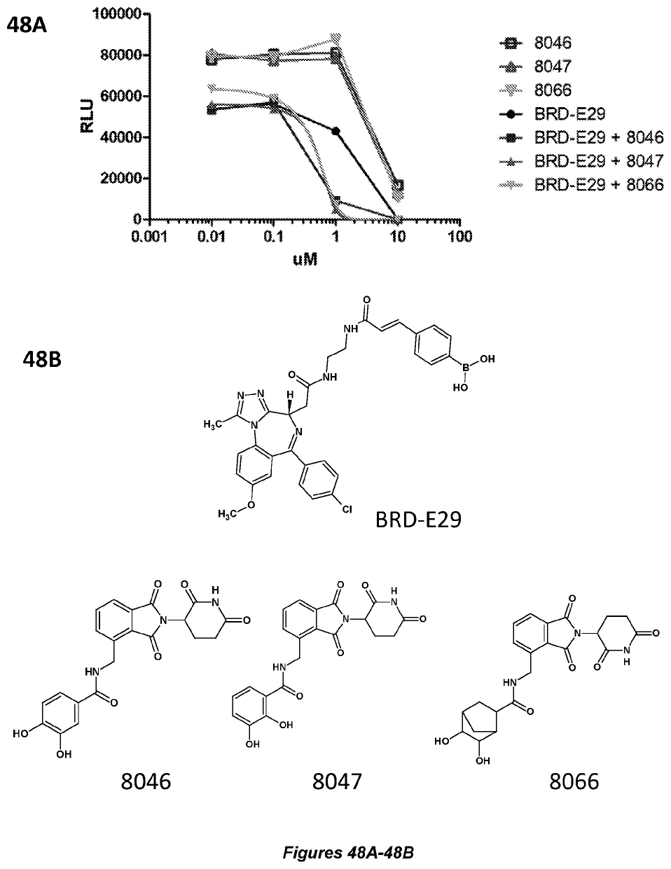

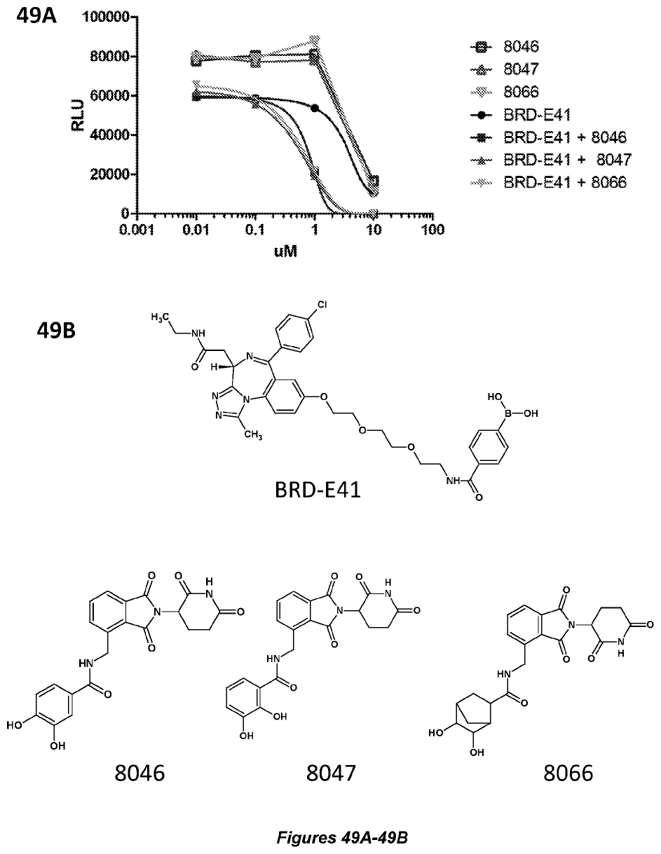

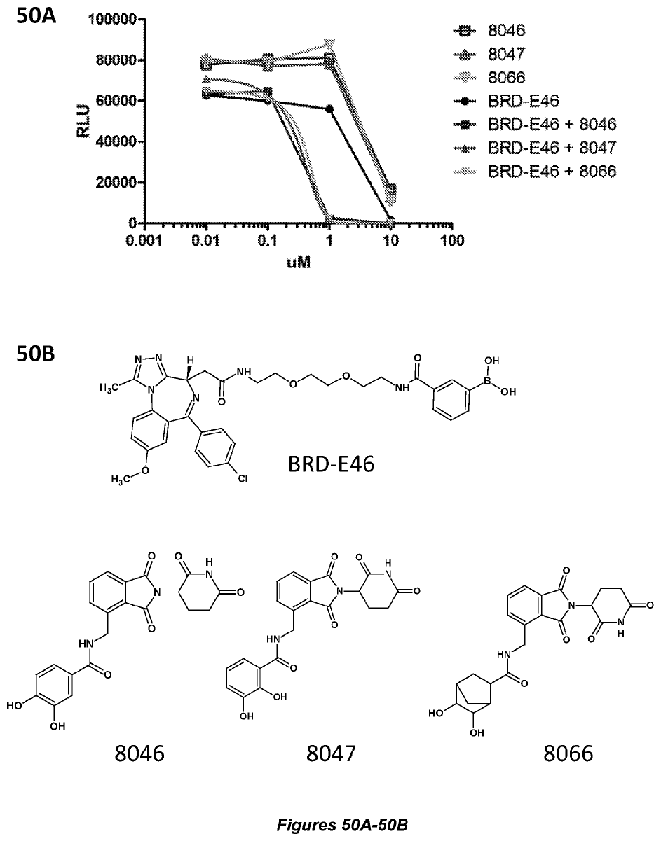

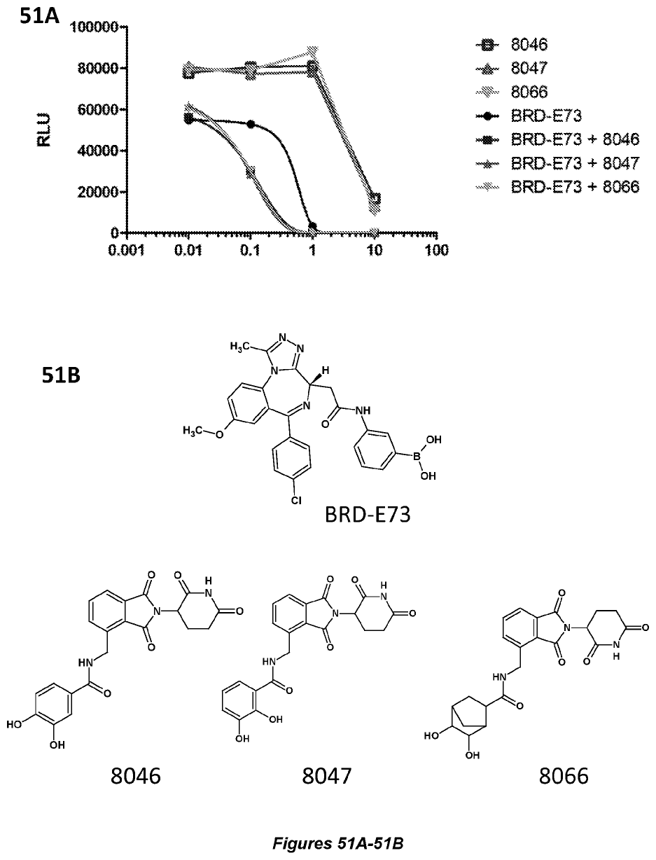

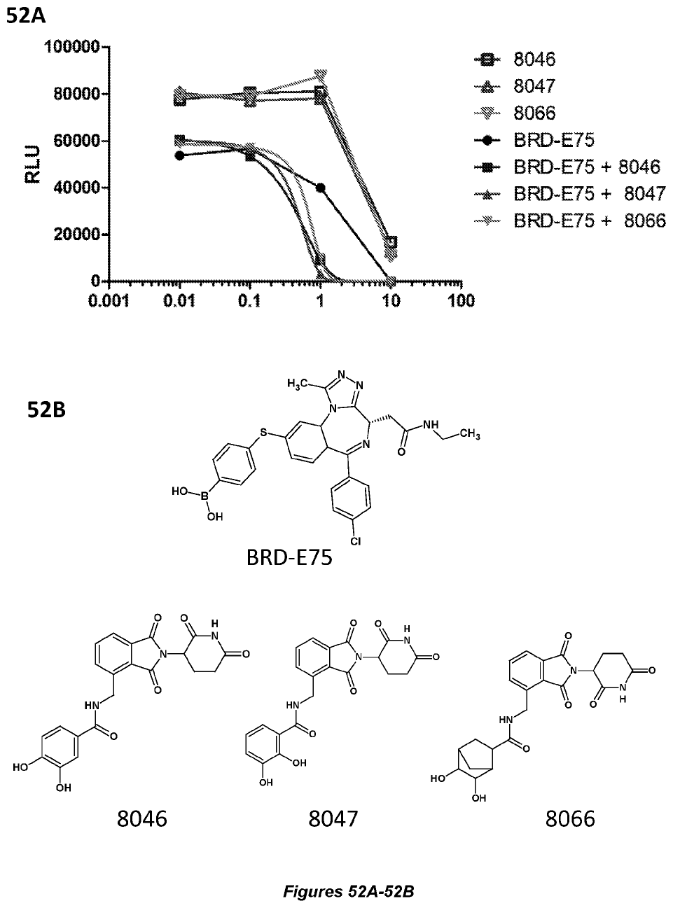

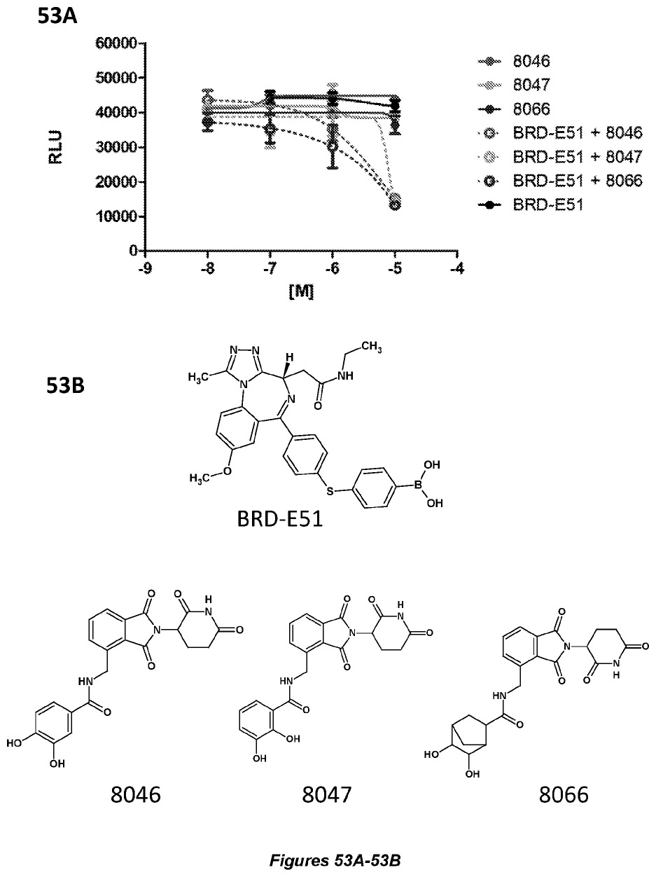

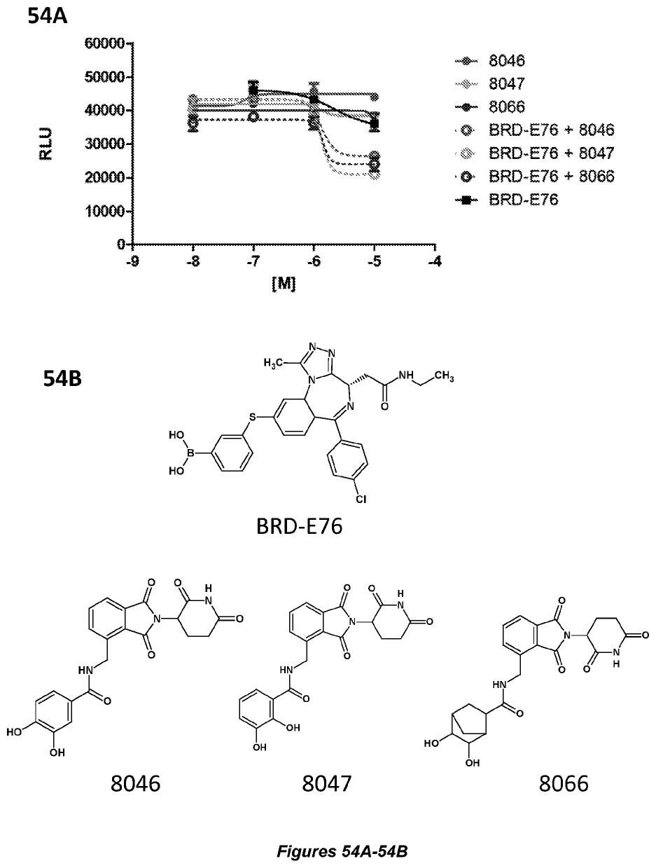

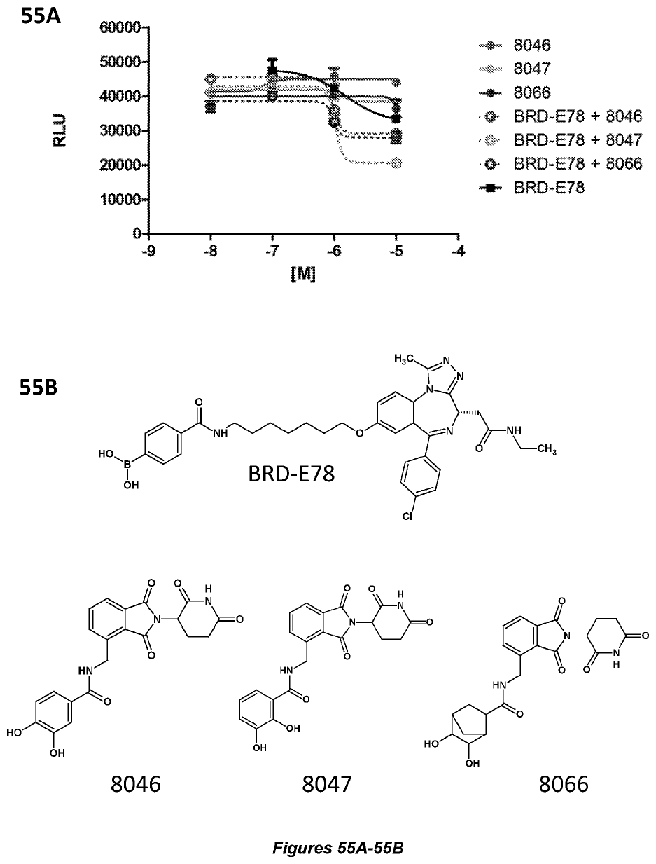

MV-4-11 cells (1×104) were treated for 72 hours with the compounds solubilized in DMSO. Compounds were added at 10 nM-100 μM each. Cellular viability was determined using the CellTiter-Glo® Luminescent Cell Viability Assay (Promega) described above. FIGS. 47-54 depict the CURE-PRO-mediated loss of cellular viability for BRD monomers (JQ1 derivatives comprising of boronic acid linkers), and CRBN ligands, 8046, 8047 and 8066 (pomalidomide derivatives comprising diol linkers) combined in a 1:1 ratio. In the presence of the BRD and CRBN monomers the dose-response curves shifted towards the left, indicating an increase in the loss of viability. All CRBN ligands and BRD ligand combinations reduced viability for BRD-E20 (FIG. 47), BRD-E29 (FIG. 48), BRD-E41 (FIG. 49), BRD-E46 (FIG. 50), BRD-E73 (FIG. 51), BRD-E75 (FIG. 52), BRD-E51 (FIG. 53), BRD-E76 (FIG. 54), BRD-E78 (FIG. 55), and BRD-E46, to a greater extent that the monomers alone.

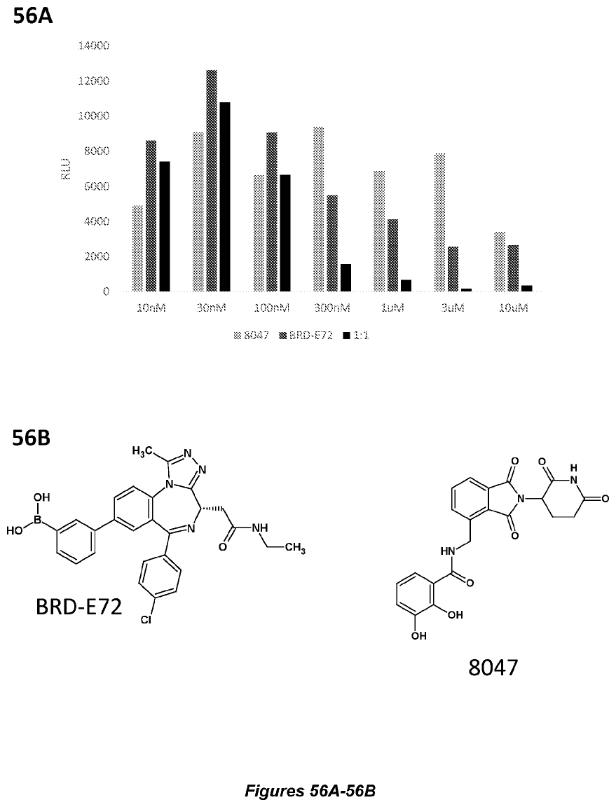

Namalwa cells (1×104) were treated for 72 hours with the compounds solubilized in DMSO. Compounds were added at 10 nM-10 μM each. Cellular viability was determined using the CellTiter-Glo® Luminescent Cell Viability Assay (Promega) described above. FIG. 56 depicts the CURE-PRO-mediated loss of cellular viability for the BRD-E72 monomer (JQ1 derivatives comprising of boronic acid linkers), and the CRBN ligand, 8047 (a pomalidomide derivative comprising diol linkers) combined in a 1:1 ratio. In the presence of the BRD and CRBN monomers, the dose-response curves shifted towards the left, indicating an increase in the loss of viability compared to monomer treatment alone.

### Example 9—CURE-PRO Increased Activation of Caspase 3/7

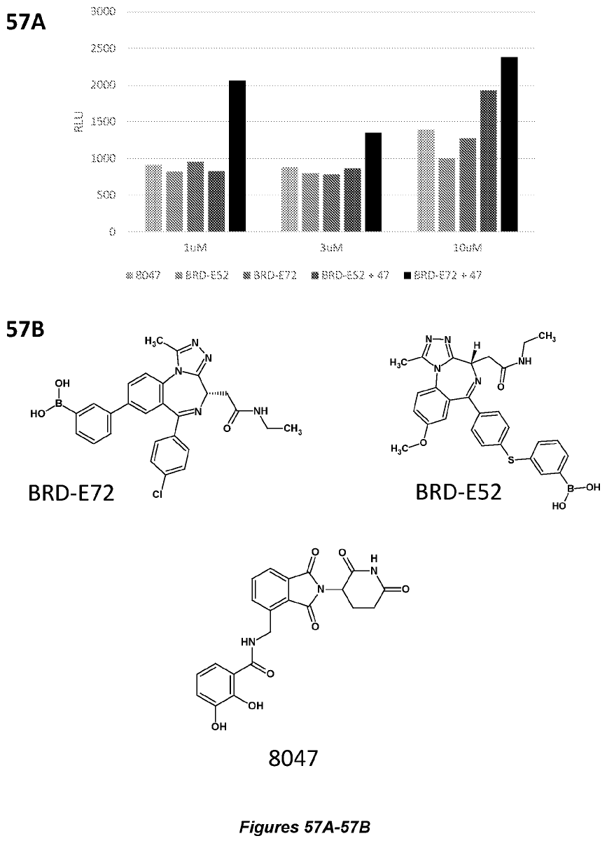

Namalwa cells (5×104) were treated for 24 hours with the compounds solubilized in DMSO. FIG. 57 is a bar graph depiction of fold increase of apoptosis assessed via caspase 3/7 activity using the Caspase-glo assay (Promega) described above. BRD-E52 or BRD-E72 together with 8047 in a 1:1 ratio, relative to BRD-E52, BRD-E72 or 8047 treatment alone, demonstrates activation of caspase 3/7.

### Example 10—Competitive Inhibition of CURE-PRO-mediated Degradation of BRD4

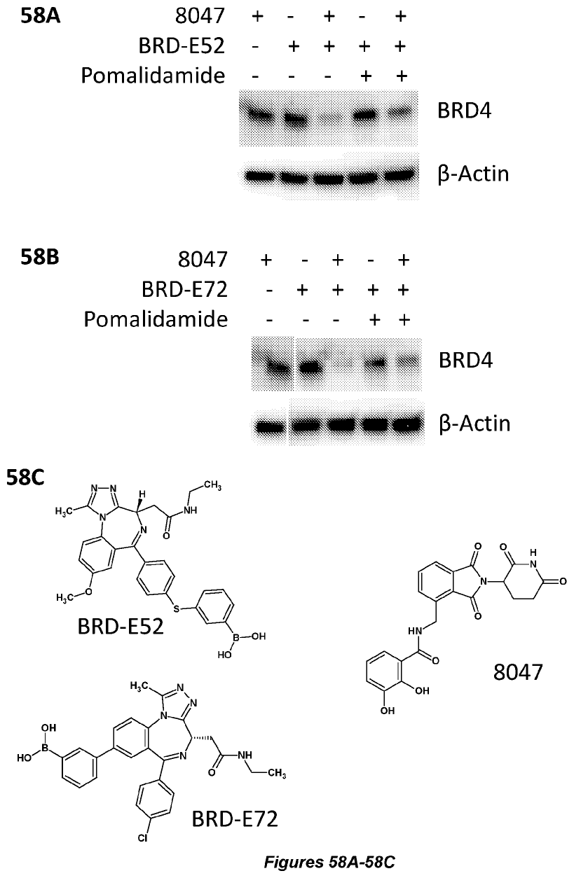

HeLa cells (2.5×10⁶) were treated for 24 h with the compounds (10 μM) solubilized in DMSO, and when used pomalidomide was preincubated with cells for 15 min at equimolar concentrations. FIG. 58 depicts that CURE-PRO mediated degradation of BRD4 can be competitively inhibited by the pre-incubation with pomalidomide, as detected by Western Blotting. CURE-PRO-mediated degradation of BRD4 with BRD-E52 (FIG. 58A) or BRD-E72 (FIG. 58B) in combination with the CRBN monomer 8047 monomer combined in a 1:1 ratio (FIGS. 58A and B, lanes 3) was not evident when cells were preincubated with pomalidomide (FIGS. 58A and B, lanes 6). Treating cells with pomalidomide and the BRD ligands failed to induce BRD4 degradation (FIGS. 58A and B, lanes 4), indicating that the dimer formed between 8047 and the BRD ligands mediates the degradation.

### Example 11—Concentration-Dependence of CURE-PRO-Mediated BRD Degradation Using MDM2 Ligands

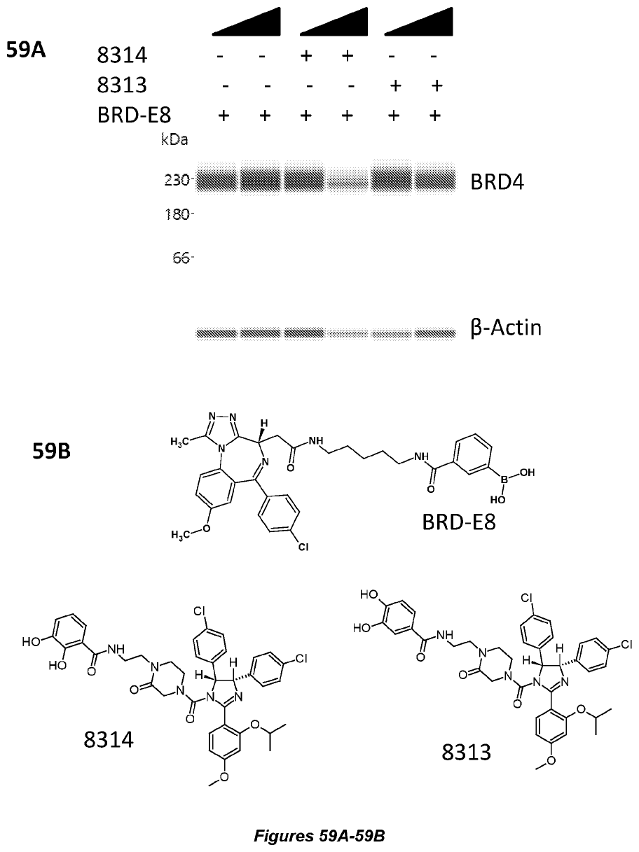

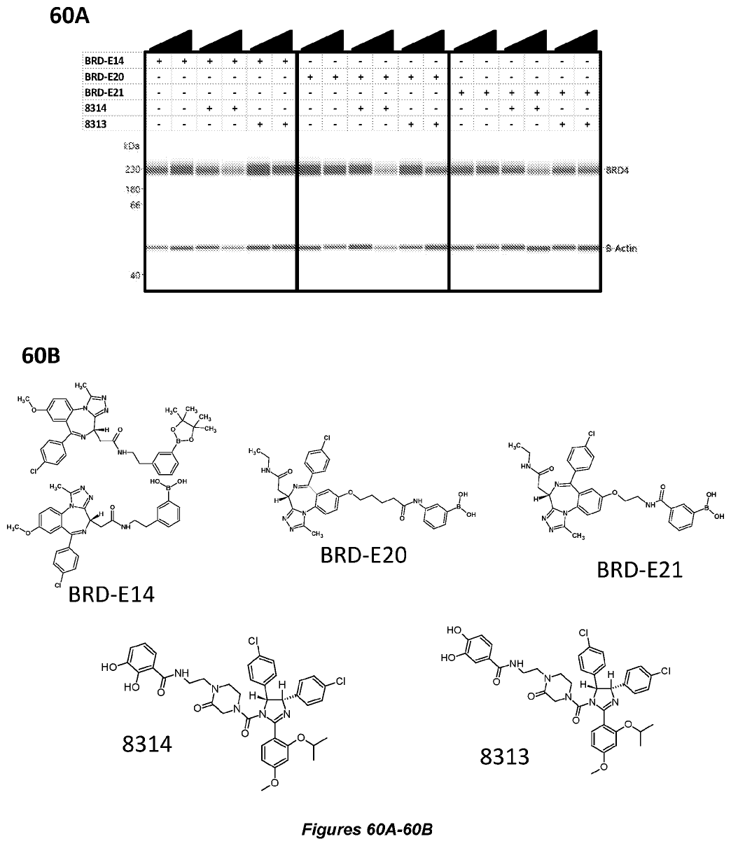

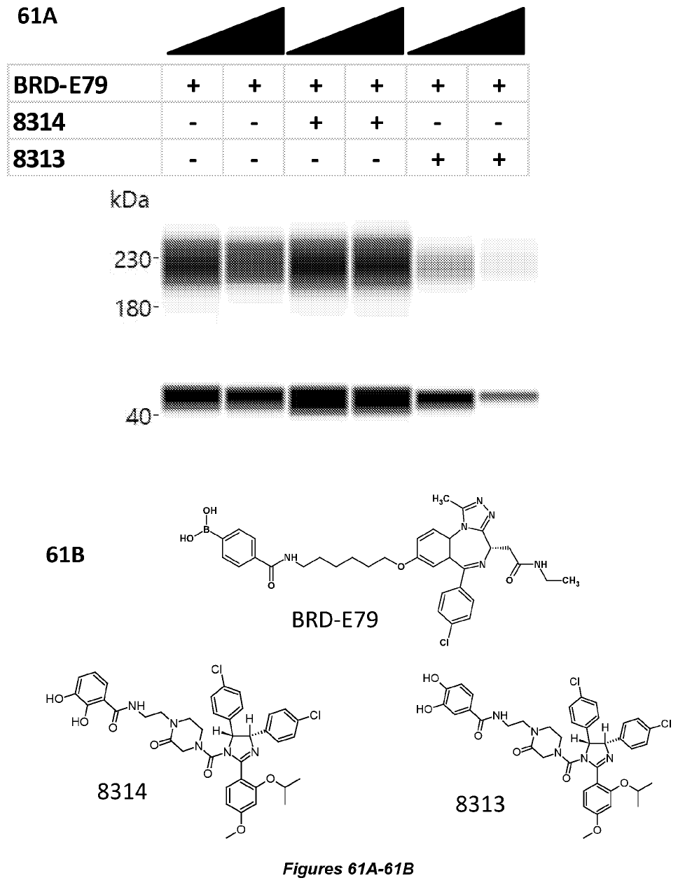

HCT116 cells (3×10⁶) were treated for 24 hours with the compounds solubilized in DMSO. Compounds were added at 1 μM-10 μM each. Standardized protein samples were electrophoresed and imaged as described in the WES ProteinSimple section above. FIGS. 59, 60, and 61 depict the concentration-dependence of CURE-PRO-mediated BRD4 degradation using MDM2 ligands. The BRD ligands (BRD-E8, FIG. 59; BRD-E14, BRD-E20 BRD-E21, FIG. 60) in combination with the MDM2 binding ligands 8314 (nutlin3a derivatives containing catechol linkers) in a 1:1 ratio at 10 μM, caused partial loss of the BRD4 protein, whereas the MDM2 ligand, 8313, had no effect on BRD4 protein levels. BRD-E79 (FIG. 61) in combination 8313 caused BRD4 protein degradation at 1 and 10 μM, but no protein loss was observed for BRD-E79 in combination with 8314.

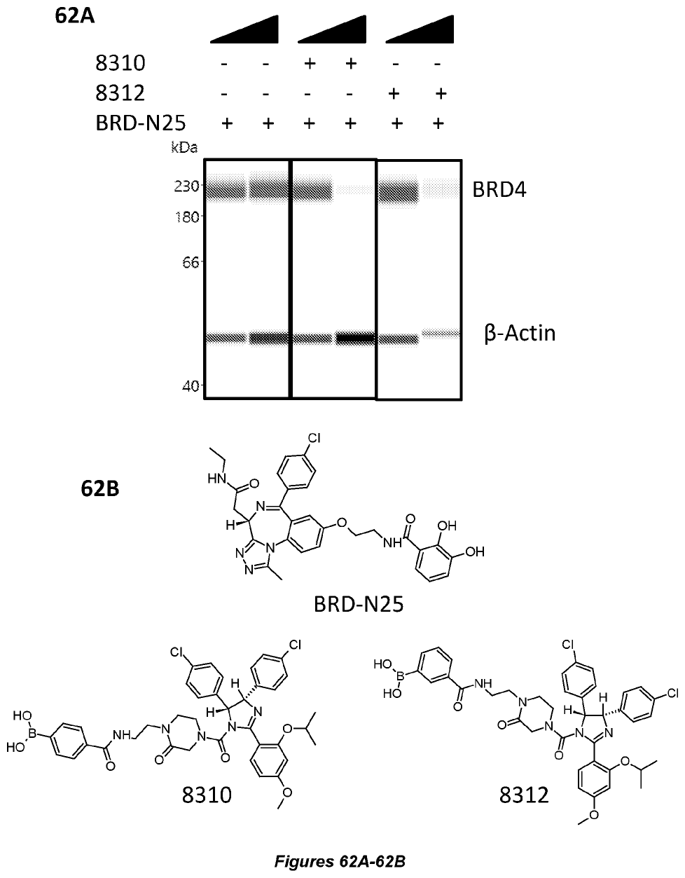

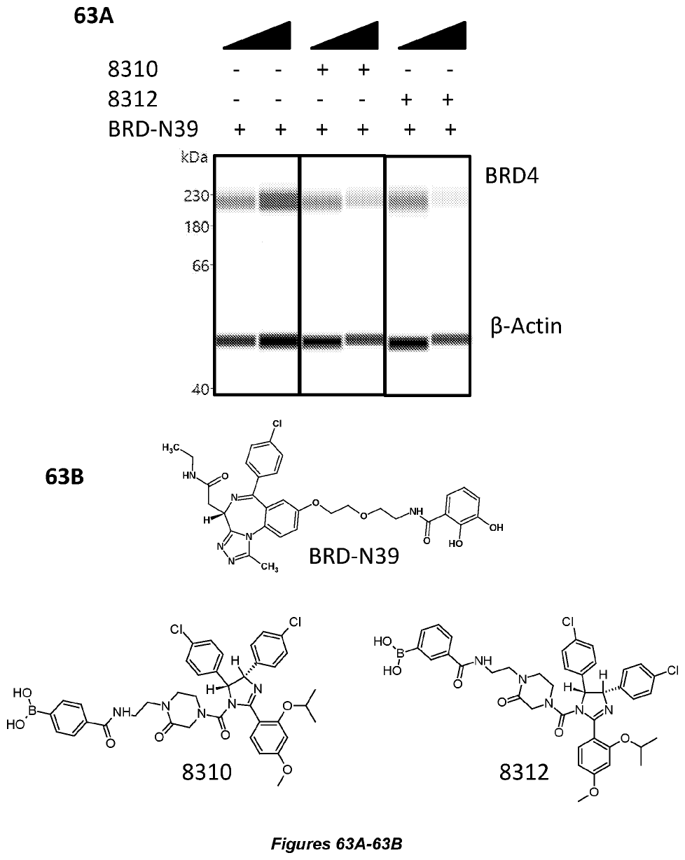

HCT116 cells (3×10⁶) were treated for 24 hours with the compounds solubilized in DMSO. Compounds were added at 1 μM-10 μM each. Standardized protein samples were electrophoresed and imaged as described in the WES ProteinSimple section above. FIGS. 62 and 63 depict the concentration-dependence of CURE-PRO-mediated BRD4 degradation using MDM2 ligands. The BRD ligands (BRD-N25, FIG. 62; and BRD-N39, FIG. 63) in combination with the MDM2 binding ligands 8310 and 8312 (nutlin3a derivatives containing boronic acid linkers) in a 1:1 ratio at 10 μM caused BRD4 degradation

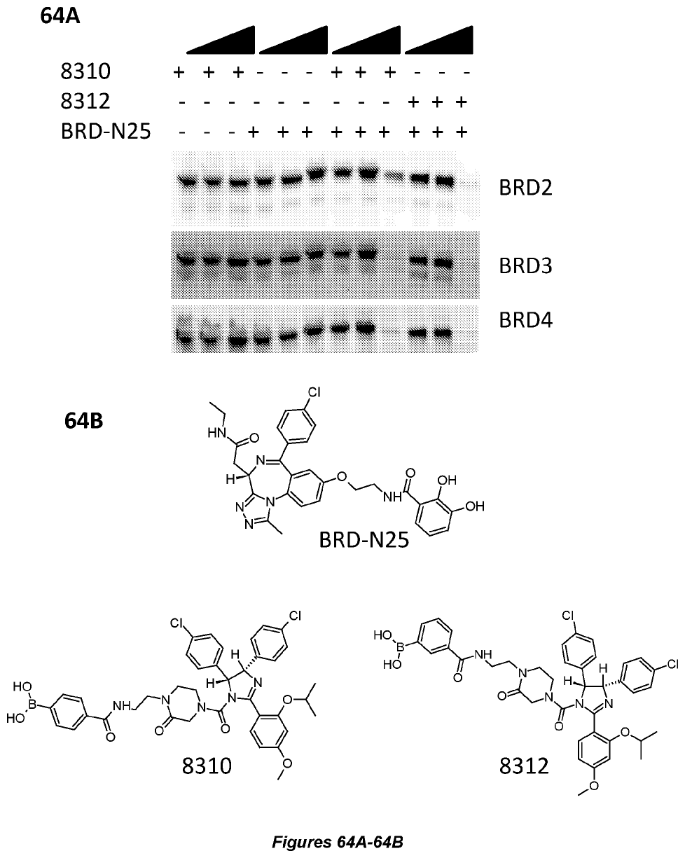

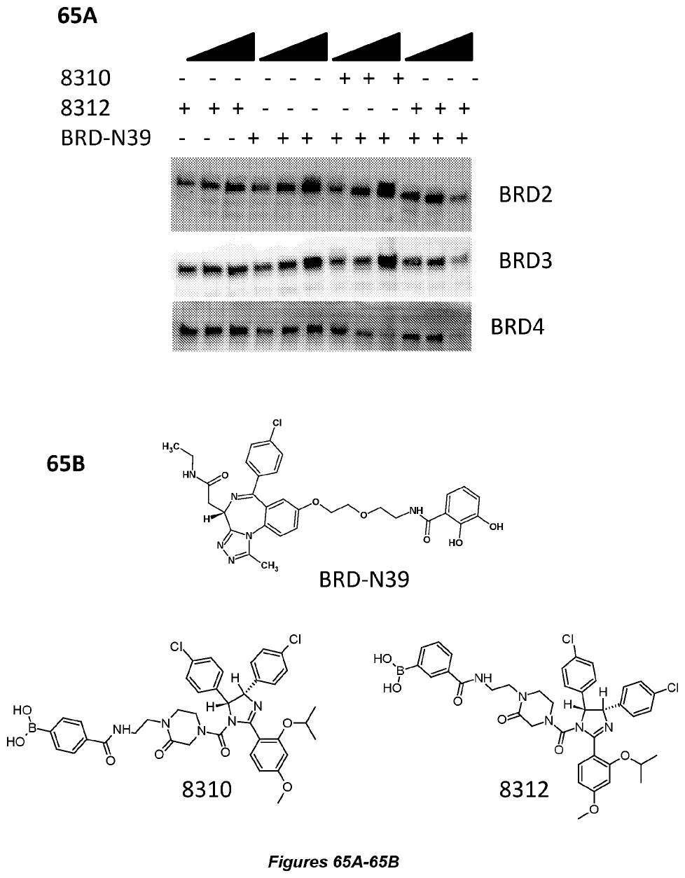

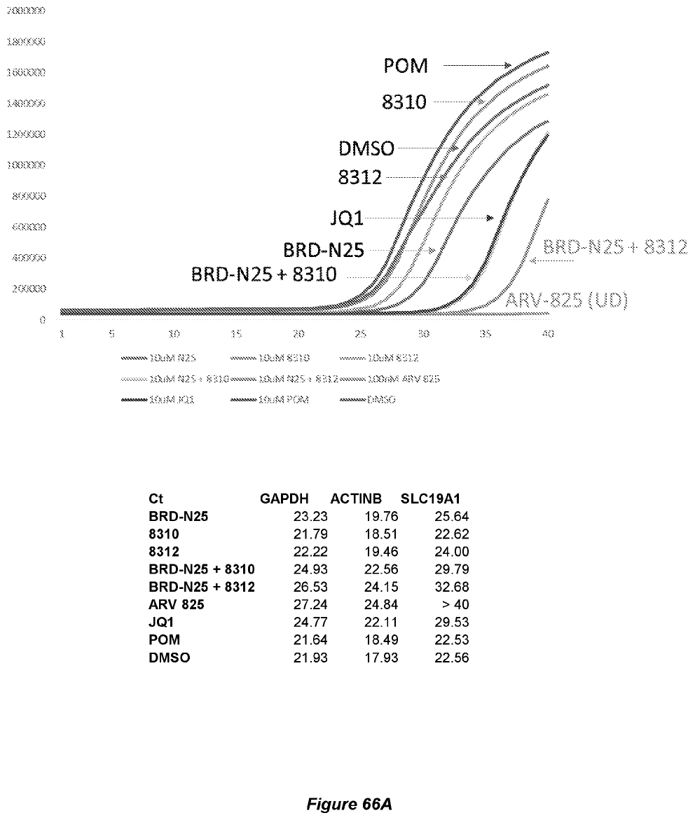

HCT116 cells (3×10⁶) were treated for 24 hours with the compounds solubilized in DMSO. Compounds were added at 100 nM-10 μM each. Standardized protein samples were electrophoresed and imaged as described in the Immunoblotting section above. FIGS. 64 and 65 depict the concentration-dependence of CURE-PRO-mediated BRD2 and BRD3 degradation using MDM2 ligands. The BRD ligands (BRD-N25, FIG. 64; and BRD-N39, FIG. 65) in combination with the MDM2 binding ligands 8310 and 8312 (nutlin3a derivatives containing boronic acid linkers) in a 1:1 ratio at 10 μM caused BRD4 degradation as well as degradation of BRD3, and BRD2 to a lesser extent.

### Example 12—CURE-PRO-Mediated Suppression of Downstream Target Gene of c-MYC

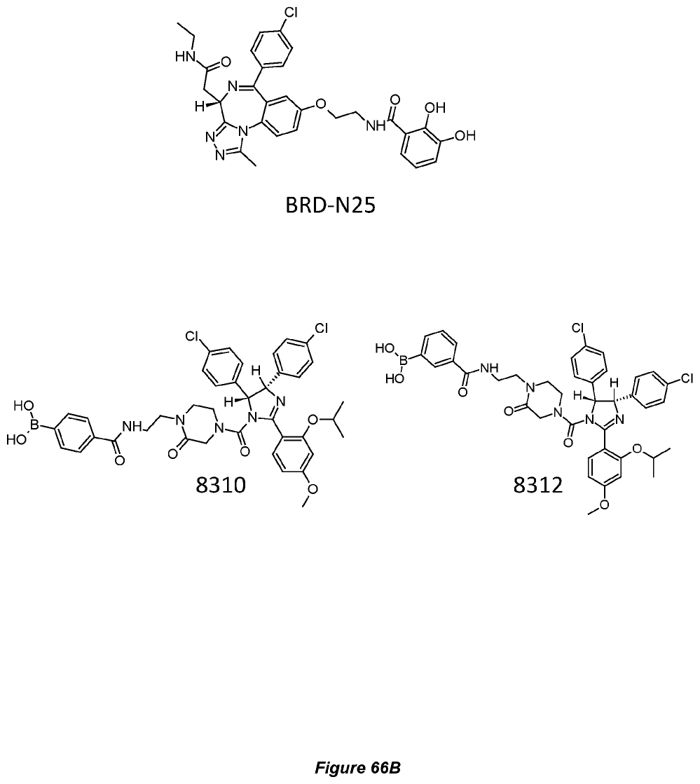

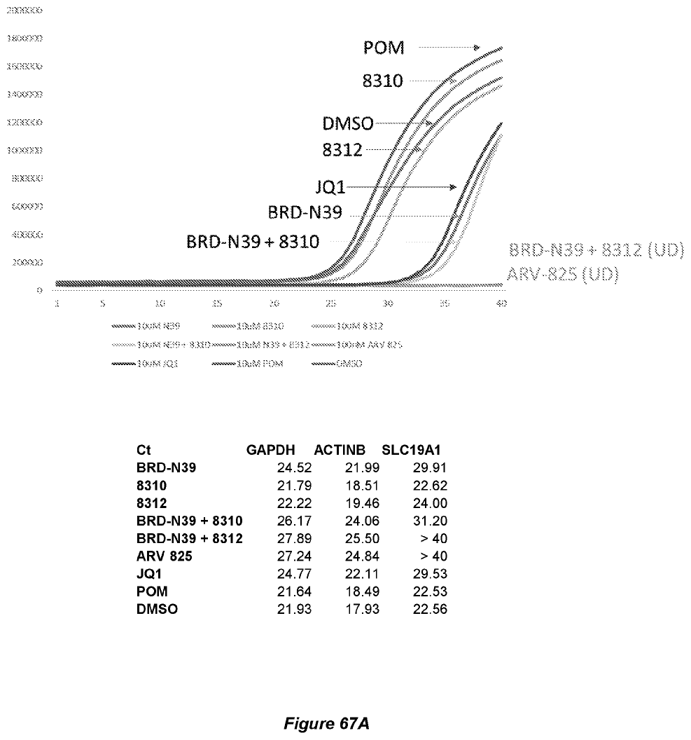

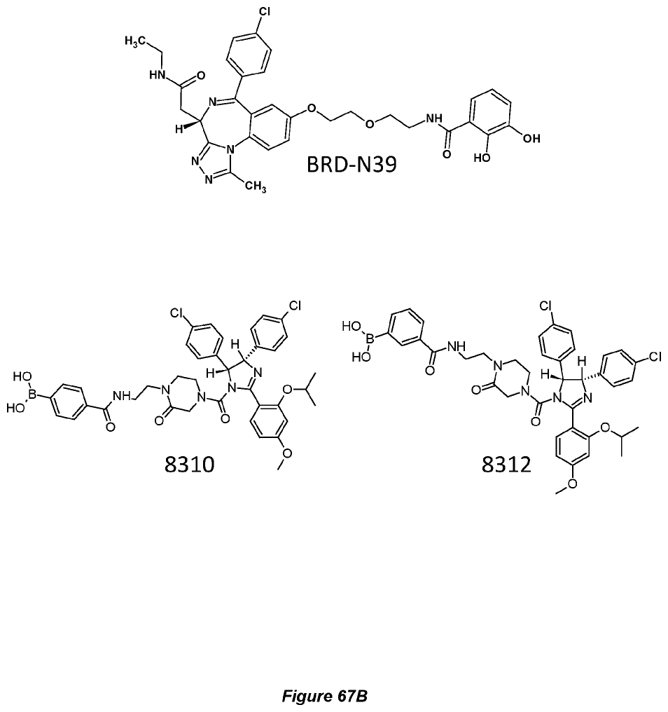

HeLa cells (1×104) were treated for 24 hours in 96 well plates with the compounds solubilized in DMSO. Compounds were added at 10 μM each and RNA expression measured by the Cells-to Ct method described above (Thermofisher). CURE-PRO-mediated suppression of the downstream target gene of c-MYC, SLC19A1, after BRD4 degradation was evident in cells treated with the combination of BRD-N25 and 8310 or 8312 (FIG. 66) and BRD-N39 and 8310 and 8312 (FIG. 67) as indicated by a rightward shift in the curve and in increase in the Ct values. Complete suppression of SLC19A1 was evident after treatment with the ARV-825 compound, which was used as a positive control, and BRD-N39 and 8312.

### Example 13—Dependence of the Proteasome for CURE-PRO-mediated BRD4 Degradation

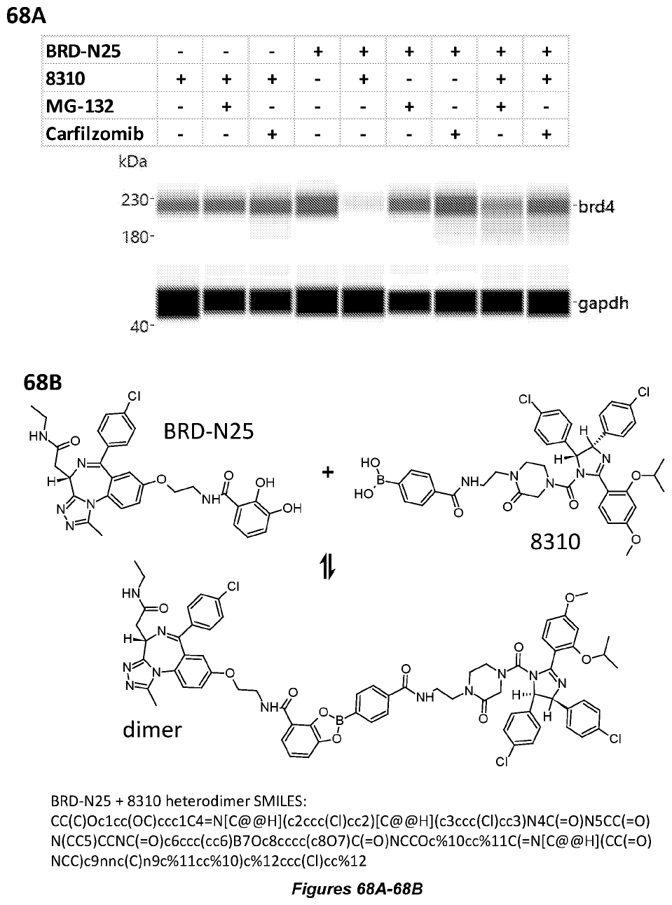

HCT116 cells (3×10⁶) were treated for 24 hours with the compounds solubilized in DMSO. CURE-PRO monomers were added at 10 μM each and the proteasomal inhibitors at 1 μM. Standardized protein samples were electrophoresed and imaged as described in the WES ProteinSimple section above. FIG. 68 depicts the dependence of the proteasome for CURE-PRO-mediated BRD4 degradation. The BRD ligands (BRD-N25) in combination with the MDM2 binding ligands 8310 (a nutlin3a derivative containing boronic acid linkers) in a 1:1 ratio at 10 μM caused BRD4 degradation, that was inhibited by the pre-incubation with MG-132 and Carfilzomib.

### Example 14—Impact of the VHL Ligand on CURE-PRO-mediated BRD4 Degradation

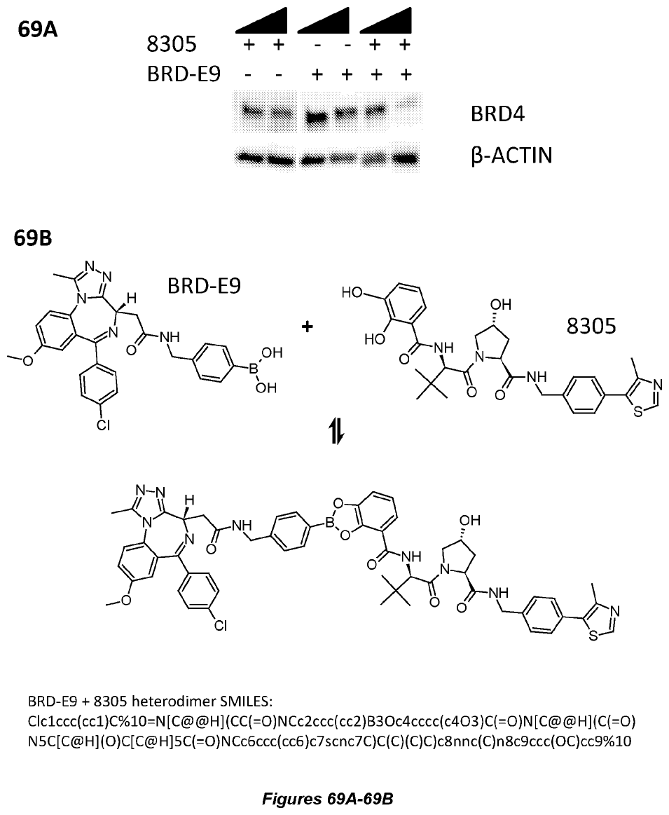

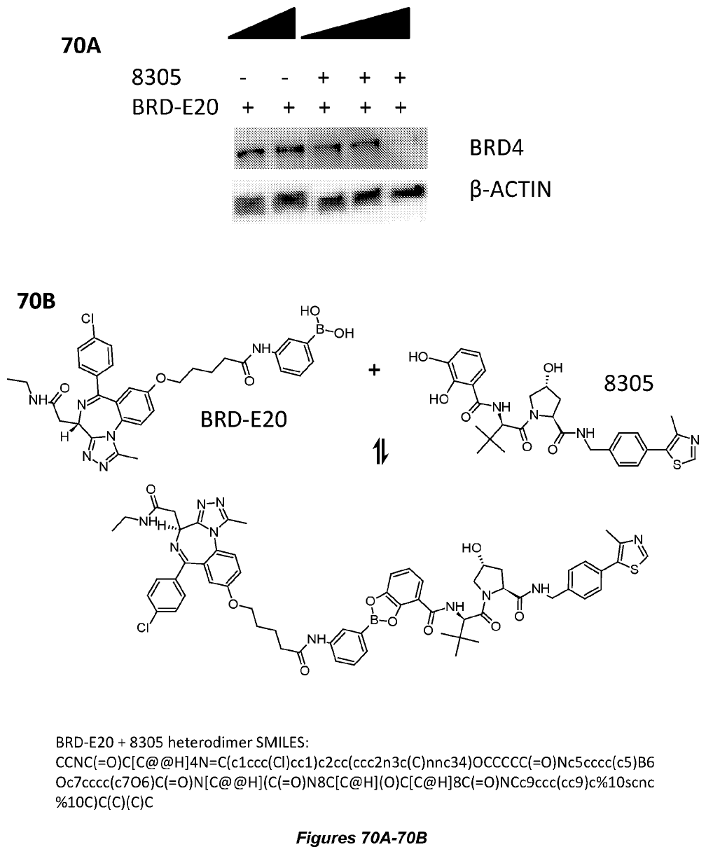

MCF7 cells (3×10⁶) were treated for 24 h with the compounds solubilized in DMSO. Compounds were added at 100 nM-10 μM. Standardized protein samples were electrophoresed and imaged as described in the Immunoblotting section above. FIG. 69 depicts the CURE-PRO-mediated BRD4 degradation for the BRD-E9 monomer and VHL ligands 8305 combined in a 1:1 ratio, and FIG. 70 depicts the CURE-PRO-mediated BRD4 degradation for the BRD-E20 monomer and 8305. In the presence of the BRD4 inhibitors (BRD-E9 and BRD-E20, JQ1 derivatives comprising boronic acid linkers) with the VHL ligand, 8305, there is degradation of BRD4 (FIG. 69: lane 6; FIG. 70: lane 5).

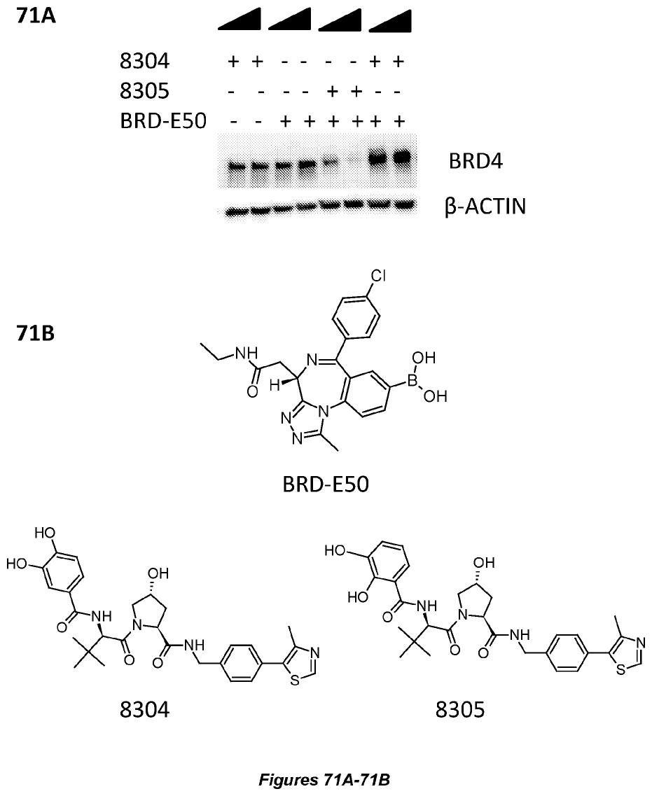

MCF7 cells (3×10⁶) were treated for 24 h with the compounds solubilized in DMSO. Compounds were added at 1 μM-10 μM. Standardized protein samples were electrophoresed and imaged as described in the Immunoblotting section above. FIG. 71 depicts the CURE-PRO-mediated BRD4 degradation for the BRD-E50 monomer and VHL ligands 8305 and 8304 combined in a 1:1 ratio. In the presence of the BRD inhibitors (BRD-E50, JQ1 derivatives comprising boronic acid linkers) with the VHL ligand, 8305, there is degradation of BRD4 (FIG. 71: lanes 5 and 6), but not when VHL ligand 8304 is co-incubated (FIG. 71: lanes 7 and 8).

### Example 15—Inhibition of CURE-PRO-mediated BRD4 Degradation

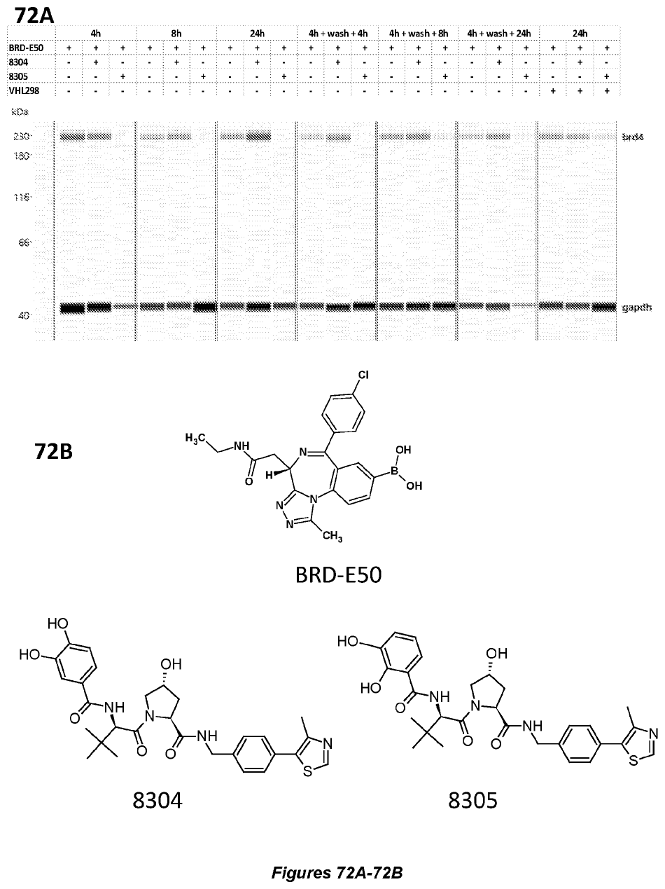

MCF7 cells (3×10⁶) were treated for 4-24 hours continually (FIG. 72), or treated for 4h, washed and incubated for a further 4-24 h with the compounds solubilized in DMSO. Compounds were added at 10 μM each. Cells were washed, lysed, and clarified by centrifugation. The total protein concentration was quantified by the BCA method (ThermoFisher Sciences). Standardized protein samples were electrophoresed and imaged as described in the WES Proteinsimple section above. FIG. 69 depicts the CURE-PRO-mediated BRD4 degradation for the BRD-E50 monomer, and VHL ligand, 8305. In the presence of only the BRD4 inhibitor (BRD-E50, JQ1 derivatives comprising boronic acid linkers) there is no degradation of BRD4 (FIG. 72: lanes 1, 4, 7, 10, 13, 16, 19). When both 8305 (a VHL298 derivative comprising a catechol linker) and BRD-E50 are present, the CURE-PRO dimer forms and directs degradation of BRD4 as early as 4 h, with sustained degradation 24 h after wash out (FIG. 72, lanes 3, 6, 9, 12, 15, 18, 21). No degradation is noted when VHL ligand 8304 are co-incubated with BRD-E50 (FIG. 72: lanes 2, 5, 8, 11, 14, 17, 20). Pre-incubation with VHL298 attenuates CURE-PRO mediated BRD4 degradation (FIG. 72, lanes 19-21).

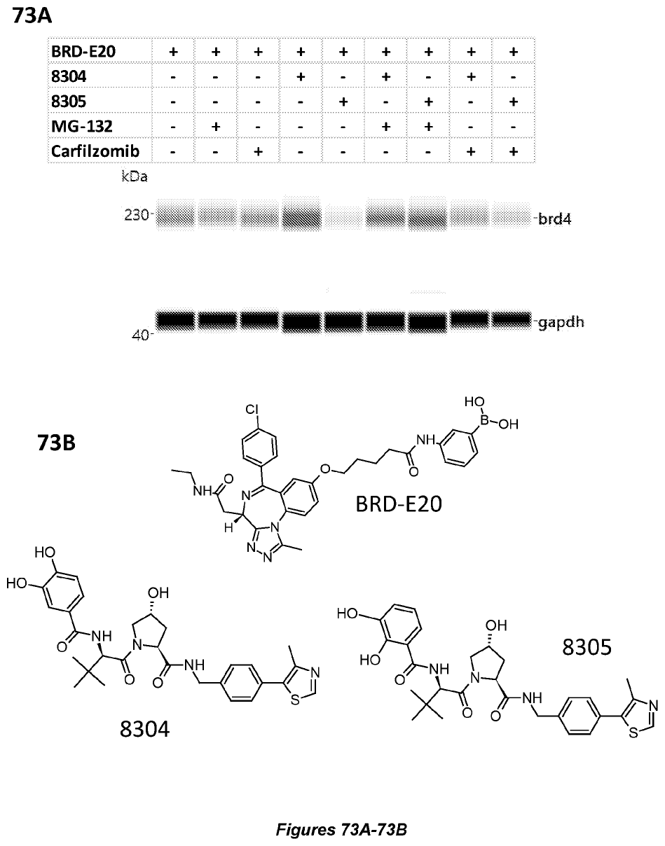

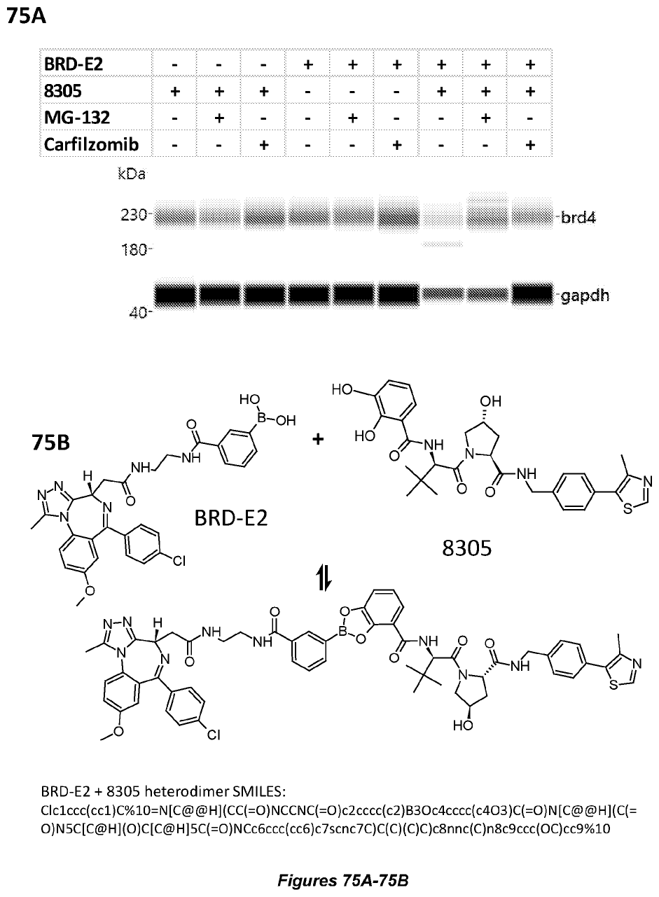

MCF7 cells (3×10⁶) were treated for 24 hours with the compounds solubilized in DMSO. CURE-PRO monomers were added at 10 μM each and the proteasomal inhibitors at 1 μM. Standardized protein samples were electrophoresed and imaged as described in the WES Proteinsimple section above. FIGS. 73 and 75 depicts the dependence of the proteasome for CURE-PRO-mediated BRD4 degradation. The BRD ligands (BRD-E20, FIG. 73 and BRD-E2, FIG. 75) in combination with the VHL binding ligands 8305 (a VHL298 derivative containing diol linkers) in a 1:1 ratio at 10 μM caused BRD4 degradation, that was inhibited by the pre-incubation with MG-132 and Carfilzomib. BRD-E20 co-incubated with 8304 failed to induce BRD4 degradation.

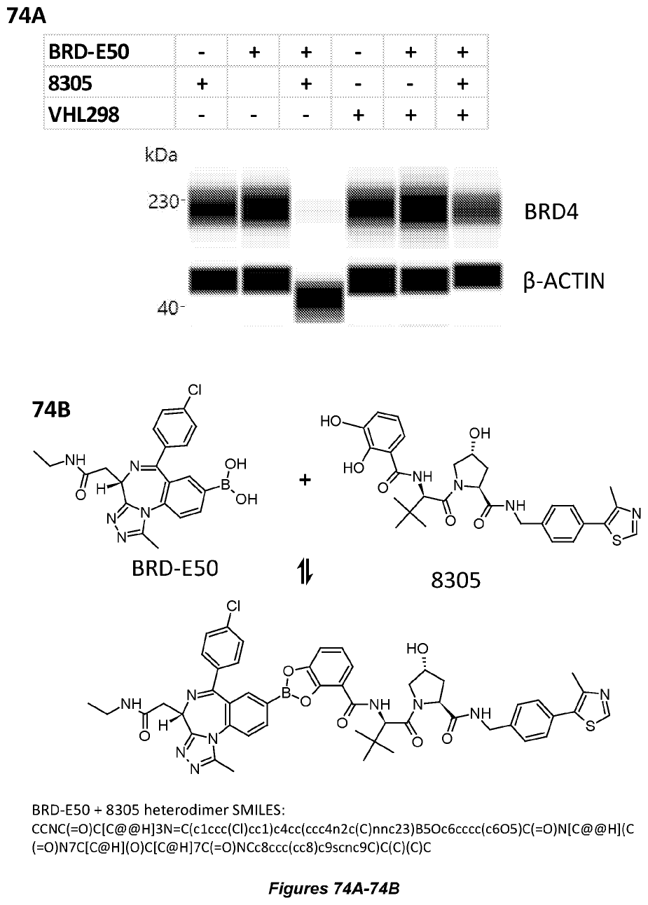

MCF7 cells (3×10⁶) were treated for 24 hours with the compounds solubilized in DMSO. CURE-PRO monomers were added at 10 μM. FIG. 74 depicts the CURE-PRO-mediated BRD4 degradation for the BRD-E50 monomer, and VHL ligand, 8305 combined in a 1:1 ratio after 24 h. In the presence of only the BRD4 inhibitor (BRD-E50, JQ1 derivatives comprising boronic acid linkers) there is no degradation of BRD4 (FIG. 74: lane 2). In the presence of only the VHL inhibitor (8305 derivatives comprising catechol linkers) there is no degradation of BRD4 (FIG. 74: lane 1). When both 8305 and BRD-E50 are present, the CURE-PRO dimer forms and directs degradation of BRD4 (FIG. 74: lane 3). No degradation is noted when VHL ligand VHL298 is preincubated in cells prior to treatment with BRD-E50 and 8305 (FIG. 74, lane 6).

### Example 16—CURE-PRO Increased Activation of Caspase 3/7

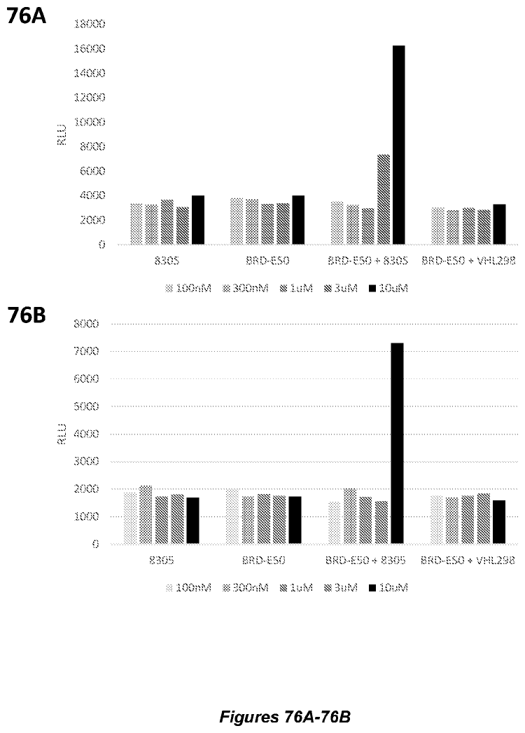

Molm-13 (FIG. 76A) or Namalwa (FIG. 76B) cells (5×10⁴) were treated for 24 hours with the compounds solubilized in DMSO in white 96 well plates (10 nM-10 μM solubilized in DMSO). FIG. 76 is a bar graph depiction of fold increase of apoptosis assessed via caspase 3/7 activity using the Caspase-glo assay (Promega) described above. BRD-E50 together with 8305 in a 1:1 ratio, relative to BRD-E50, or 8305 treatment alone, demonstrates activation of caspase 3/7.

### Example 17—Impact of CURE-PRO Ligand Co-dosing on Cellular Viability

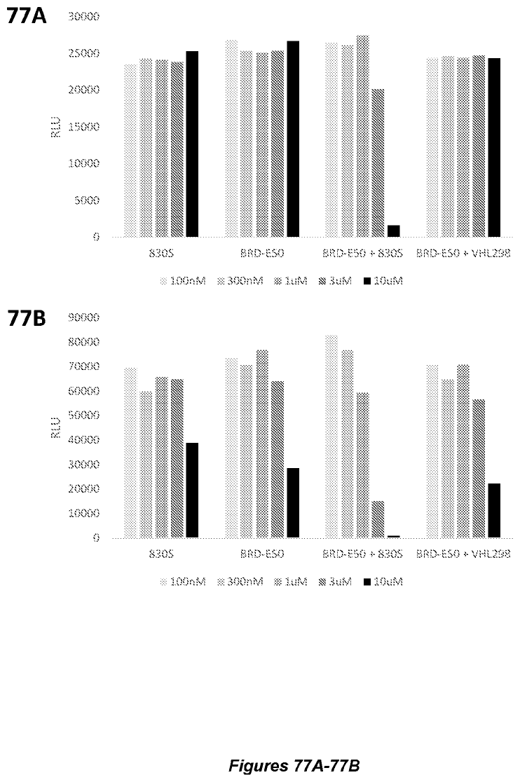

Molm-13 cells (1×10⁴) were treated for 24 (FIG. 77A) or 72 (FIG. 77B) hours were treated in white 96 well plates with the compounds (10 nM-10 μM solubilized in DMSO). After indicated times cell viability was assessed using the CellTiter-Glo® Luminescent Cell Viability Assay (Promega) and the signal detected on a SpectraMax M5 (Molecular Devices). Co-dosing BRD-E50 with the VHL ligand, 8305, at a 1:1 ratio demonstrates marked loss in cell viability when compared to monomer treatment alone. Co-treatment for BRD-E50 with the VHL ligand, VHL298, which is incapable of forming a heterobivalent molecule, decreases levels of cell viability to similar levels of BRD-E50 treatment alone.

### Discussion of Examples

### Summary Tables 1-6 of E3 Ligase Directed Degradation.

The following tables provide a summary of combinations of CURE-PRO pairs with demonstrated efficacy of 30% to 70% or higher protein degradation as estimated from Western Blot or WES (Proteinsimple) analysis, as described above. A black check mark indicates efficacy of 30% to 70% or higher protein degradation.

TABLE 1
Summary of Degradation of BRD4 Using a Combination of an Aryl-boronic Acid-containing BRD4 Ligand and an Aromatic 1,2-diol- or Hindered cis- 1,2-diol-containing CRBN ligand

| LIGAND | | 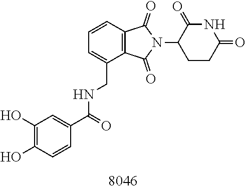 [SMILES: O=C1CCC(N2C(=O)c3cccc(CNC(=O)c4ccc(O)c(O)c4)c3C2=O)C(=O)N1] | 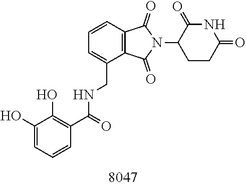 [SMILES: O=C1CCC(N2C(=O)c3cccc(CNC(=O)c4cccc(O)c4O)c3C2=O)C(=O)N1] | 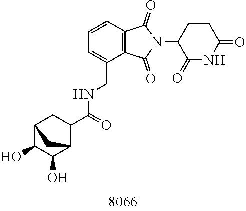 [SMILES: O=C1CCC(N2C(=O)c3cccc(CNC(=O)C4C[C@@H]5C[C@H]4[C@@H](O)[C@H]5O)c3C2=O)C(=O)N1] |
|---|---|---|---|---|
| BRD- E04 | 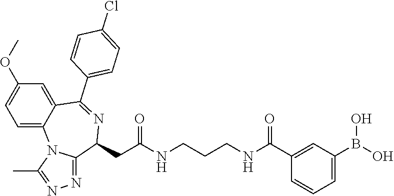 [SMILES: COc1ccc2c(c1)C(c1ccc(Cl)cc1)=N[C@@H](CC(=O)NCCCNC(=O)c1cccc(B(O)O)c1)c1nnc(C)n1-2] | ✓ | ✓ | ✓ |
| BRD- E05 | 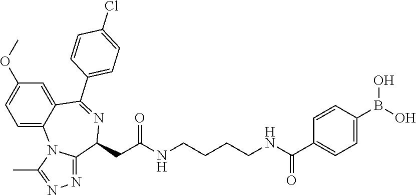 [SMILES: COc1ccc2c(c1)C(c1ccc(Cl)cc1)=N[C@@H](CC(=O)NCCCCNC(=O)c1ccc(B(O)O)cc1)c1nnc(C)n1-2] | | ✓ | ✓ |
| BRD- E07 | 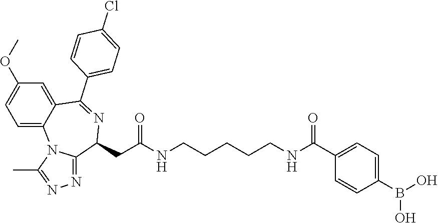 [SMILES: COc1ccc2c(c1)C(c1ccc(Cl)cc1)=N[C@@H](CC(=O)NCCCCCNC(=O)c1ccc(B(O)O)cc1)c1nnc(C)n1-2] | | | ✓ |
| BRD- E08 | 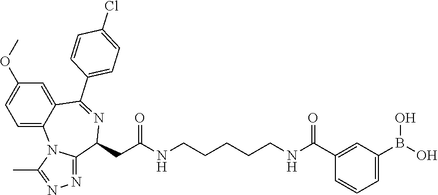 [SMILES: COc1ccc2c(c1)C(c1ccc(Cl)cc1)=N[C@@H](CC(=O)NCCCCCNC(=O)c1cccc(B(O)O)c1)c1nnc(C)n1-2] | ✓ | | ✓ |
| BRD- E09 | 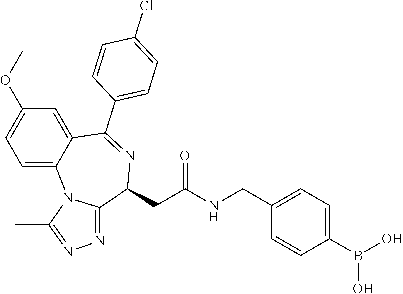 [SMILES: COc1ccc2c(c1)C(c1ccc(Cl)cc1)=N[C@@H](CC(=O)NCc1ccc(B(O)O)cc1)c1nnc(C)n1-2] | | ✓ | |
| BRD- E10 | 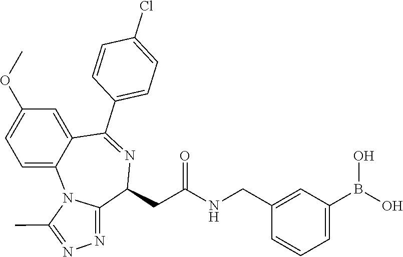 [SMILES: COc1ccc2c(c1)C(c1ccc(Cl)cc1)=N[C@@H](CC(=O)NCc1cccc(B(O)O)c1)c1nnc(C)n1-2] | ✓ | ✓ | |
| BRD- E14 | 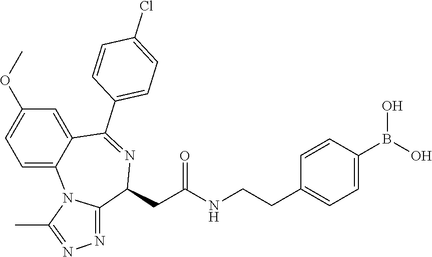 [SMILES: COc1ccc2c(c1)C(c1ccc(Cl)cc1)=N[C@@H](CC(=O)NCCc1ccc(B(O)O)cc1)c1nnc(C)n1-2] | | ✓ | |
| BRD- E20 | 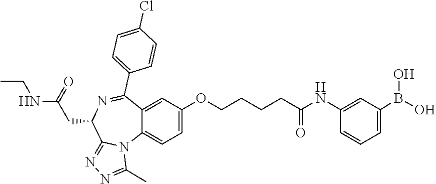 [SMILES: CCNC(=O)C[C@@H]1N=C(c2ccc(Cl)cc2)c2cc(OCCCCC(=O)Nc3cccc(B(O)O)c3)ccc2-n2c(C)nnc21] | ✓ | | ✓ |
| BRD- E21 | 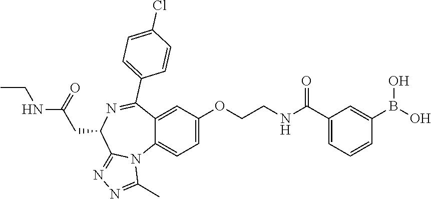 [SMILES: CCNC(=O)C[C@@H]1N=C(c2ccc(Cl)cc2)c2cc(OCCNC(=O)c3cccc(B(O)O)c3)ccc2-n2c(C)nnc21] | | ✓ | |
| BRD- E27 | 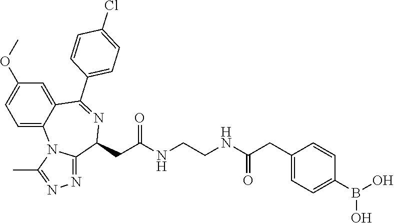 [SMILES: COc1ccc2c(c1)C(c1ccc(Cl)cc1)=N[C@@H](CC(=O)NCCNC(=O)Cc1ccc(B(O)O)cc1)c1nnc(C)n1-2] | | ✓ | |
| BRD- E29 | 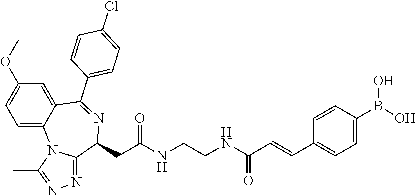 [SMILES: COc1ccc2c(c1)C(c1ccc(Cl)cc1)=N[C@@H](CC(=O)NCCNC(=O)/C=C/c1ccc(B(O)O)cc1)c1nnc(C)n1-2] | ✓ | ✓ | ✓ |
| BRD- E30 | 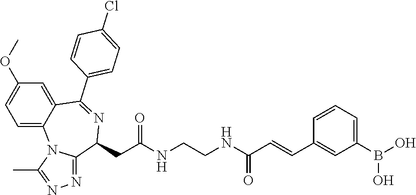 [SMILES: COc1ccc2c(c1)C(c1ccc(Cl)cc1)=N[C@@H](CC(=O)NCCNC(=O)/C=C/c1cccc(B(O)O)c1)c1nnc(C)n1-2] | | ✓ | ✓ |
| BRD- E38 | 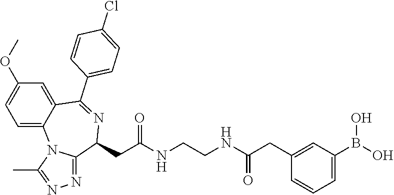 [SMILES: COc1ccc2c(c1)C(c1ccc(Cl)cc1)=N[C@@H](CC(=O)NCCNC(=O)Cc1cccc(B(O)O)c1)c1nnc(C)n1-2] | | ✓ | |
| BRD- E39 | 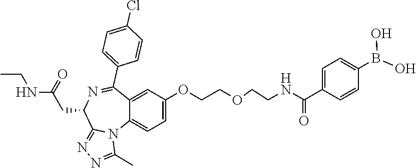 [SMILES: CCNC(=O)C[C@@H]1N=C(c2ccc(Cl)cc2)c2cc(OCCOCCNC(=O)c3ccc(B(O)O)cc3)ccc2-n2c(C)nnc21] | ✓ | | |
| BRD- E42 | 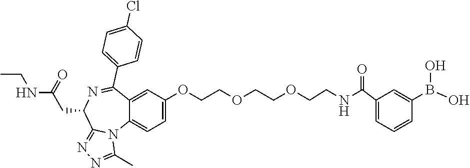 [SMILES: CCNC(=O)C[C@@H]1N=C(c2ccc(Cl)cc2)c2cc(OCCOCCOCCNC(=O)c3cccc(B(O)O)c3)ccc2-n2c(C)nnc21] | | ✓ | |
| BRD- E43 | 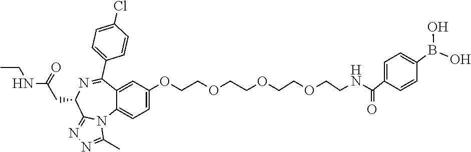 [SMILES: CCNC(=O)C[C@@H]1N=C(c2ccc(Cl)cc2)c2cc(OCCOCCOCCOCCNC(=O)c3ccc(B(O)O)cc3)ccc2-n2c(C)nnc21] | | ✓ | ✓ |
| BRD- E45 | 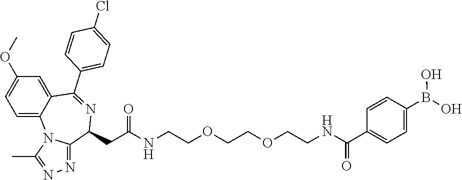 [SMILES: COc1ccc2c(c1)C(c1ccc(Cl)cc1)=N[C@@H](CC(=O)NCCOCCOCCNC(=O)c1ccc(B(O)O)cc1)c1nnc(C)n1-2] | | | ✓ |
| BRD- E46 | 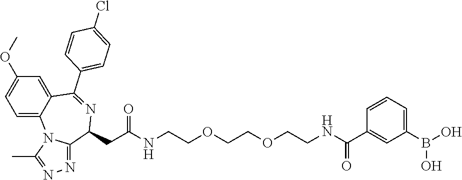 [SMILES: COc1ccc2c(c1)C(c1ccc(Cl)cc1)=N[C@@H](CC(=O)NCCOCCOCCNC(=O)c1cccc(B(O)O)c1)c1nnc(C)n1-2] | ✓ | | ✓ |
| BRD- E52 | 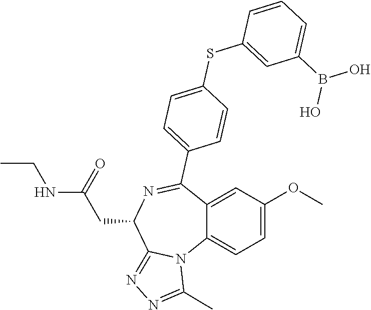 [SMILES: CCNC(=O)C[C@@H]1N=C(c2ccc(Sc3cccc(B(O)O)c3)cc2)c2cc(OC)ccc2-n2c(C)nnc21] | | ✓ | ✓ |
| BRD- E72 | 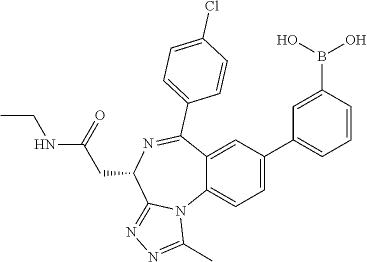 [SMILES: CCNC(=O)C[C@@H]1N=C(c2ccc(Cl)cc2)c2cc(-c3cccc(B(O)O)c3)ccc2-n2c(C)nnc21] | | ✓ | |
| BRD- E74 | 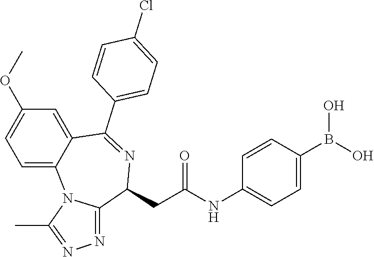 [SMILES: COc1ccc2c(c1)C(c1ccc(Cl)cc1)=N[C@@H](CC(=O)Nc1ccc(B(O)O)cc1)c1nnc(C)n1-2] | ✓ | | |
| BRD- E76 | 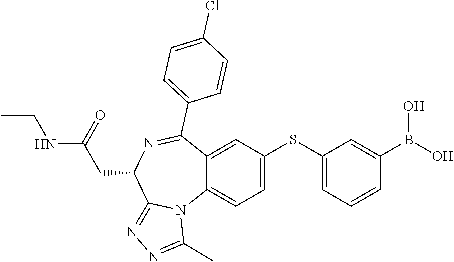 [SMILES: CCNC(=O)C[C@@H]1N=C(c2ccc(Cl)cc2)c2cc(Sc3cccc(B(O)O)c3)ccc2-n2c(C)nnc21] | ✓ | | |
| BRD- E79 | 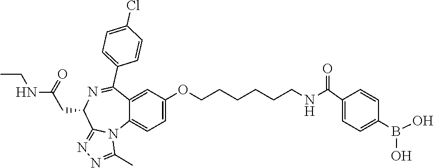 [SMILES: CCNC(=O)C[C@@H]1N=C(c2ccc(Cl)cc2)c2cc(OCCCCCCNC(=O)c3ccc(B(O)O)cc3)ccc2-n2c(C)nnc21] | ✓ | ✓ | ✓ |

TABLE 3
Summary of Degradation of BRD4 Using a Combination of an Aryl-boronic Acid-containing BRD4 Ligand and an Aromatic 1,2-diol-containing VHL Ligand.

| LIGAND | | 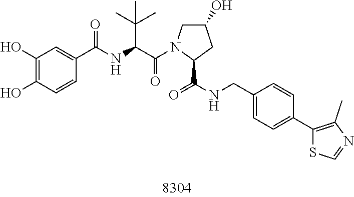 [SMILES: Cc1ncsc1-c1ccc(CNC(=O)[C@@H]2C[C@@H](O)CN2C(=O)[C@@H](NC(=O)c2ccc(O)c(O)c2)C(C)(C)C)cc1] | 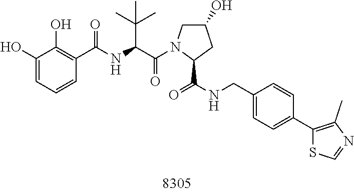 [SMILES: Cc1ncsc1-c1ccc(CNC(=O)[C@@H]2C[C@@H](O)CN2C(=O)[C@@H](NC(=O)c2cccc(O)c2O)C(C)(C)C)cc1] |
|---|---|---|---|
| BRD-E02 | 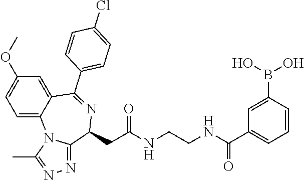 [SMILES: COc1ccc2c(c1)C(c1ccc(Cl)cc1)=N[C@@H](CC(=O)NCCNC(=O)c1cccc(B(O)O)c1)c1nnc(C)n1-2] | | ✓ |
| BRD-E09 | 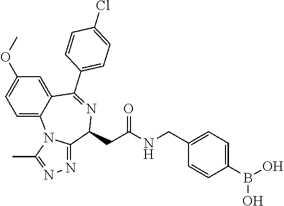 [SMILES: COc1ccc2c(c1)C(c1ccc(Cl)cc1)=N[C@@H](CC(=O)NCc1ccc(B(O)O)cc1)c1nnc(C)n1-2] | | ✓ |
| BRD-E10 | 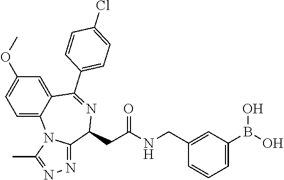 [SMILES: COc1ccc2c(c1)C(c1ccc(Cl)cc1)=N[C@@H](CC(=O)NCc1cccc(B(O)O)c1)c1nnc(C)n1-2] | ✓ | |
| BRD-E14 | 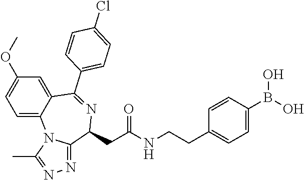 [SMILES: COc1ccc2c(c1)C(c1ccc(Cl)cc1)=N[C@@H](CC(=O)NCCc1ccc(B(O)O)cc1)c1nnc(C)n1-2] | | ✓ |
| BRD-E20 | 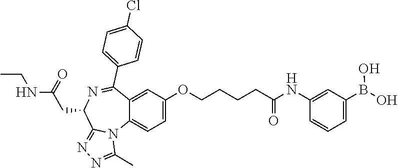 [SMILES: CCNC(=O)C[C@@H]1N=C(c2ccc(Cl)cc2)c2cc(OCCCCC(=O)Nc3cccc(B(O)O)c3)ccc2-n2c(C)nnc21] | | ✓ |
| BRD-E45 | 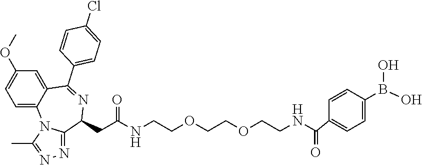 [SMILES: COc1ccc2c(c1)C(c1ccc(Cl)cc1)=N[C@@H](CC(=O)NCCOCCOCCNC(=O)c1ccc(B(O)O)cc1)c1nnc(C)n1-2] | | ✓ |
| BRD-E50 | 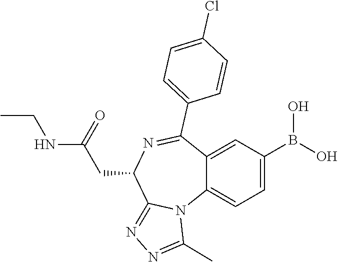 [SMILES: CCNC(=O)C[C@@H]1N=C(c2ccc(Cl)cc2)c2cc(B(O)O)ccc2-n2c(C)nnc21] | | ✓ |
| BRD-E54 | 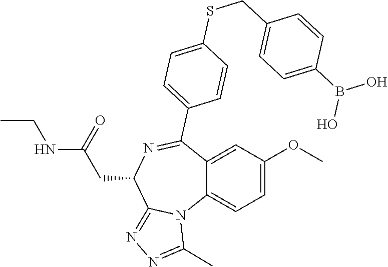 [SMILES: CCNC(=O)C[C@@H]1N=C(c2ccc(SCc3ccc(B(O)O)cc3)cc2)c2cc(OC)ccc2-n2c(C)nnc21] | | ✓ |
| BRD-E78 | 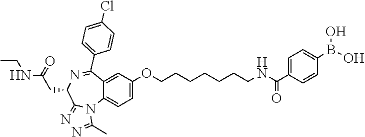 [SMILES: CCNC(=O)C[C@@H]1N=C(c2ccc(Cl)cc2)c2cc(OCCCCCCCNC(=O)c3ccc(B(O)O)cc3)ccc2-n2c(C)nnc21] | | ✓ |

TABLE 4
Summary of Degradation of BRD4 Using a Combination of an Aromatic 1,2-diol-containing BRD4 Ligand and an Aryl-boronic Acid-containing CRBN Ligand.

| LIGAND | | 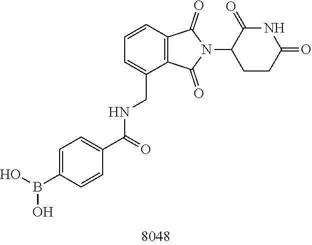 [SMILES: O=C1CCC(N2C(=O)c3cccc(CNC(=O)c4ccc(B(O)O)cc4)c3C2=O)C(=O)N1] | 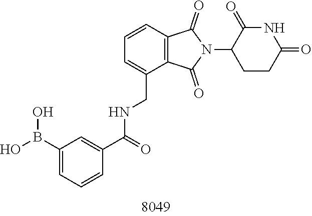 [SMILES: O=C1CCC(N2C(=O)c3cccc(CNC(=O)c4cccc(B(O)O)c4)c3C2=O)C(=O)N1] |
|---|---|---|---|
| BRD-N01 | 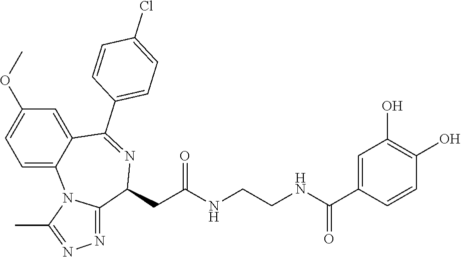 [SMILES: COc1ccc2c(c1)C(c1ccc(Cl)cc1)=N[C@@H](CC(=O)NCCNC(=O)c1ccc(O)c(O)c1)c1nnc(C)n1-2] | ✓ | |
| BRD-N05 | 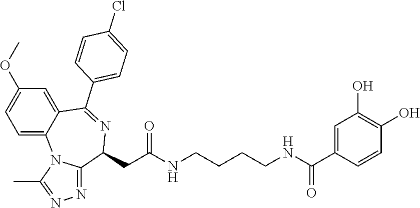 [SMILES: COc1ccc2c(c1)C(c1ccc(Cl)cc1)=N[C@@H](CC(=O)NCCCCNC(=O)c1ccc(O)c(O)c1)c1nnc(C)n1-2] | ✓ | |
| BRD-N06 | 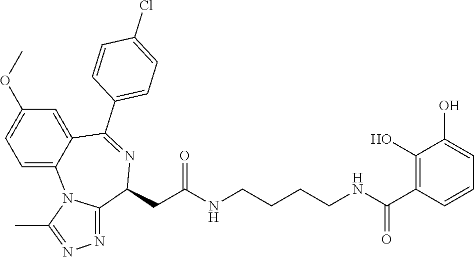 [SMILES: COc1ccc2c(c1)C(c1ccc(Cl)cc1)=N[C@@H](CC(=O)NCCCCNC(=O)c1cccc(O)c1O)c1nnc(C)n1-2] | ✓ | |
| BRD-N10 | 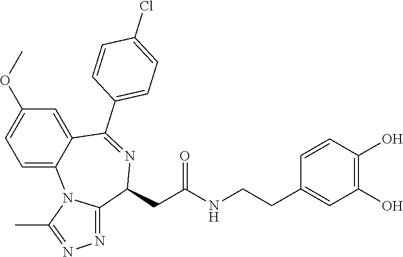 [SMILES: COc1ccc2c(c1)C(c1ccc(Cl)cc1)=N[C@@H](CC(=O)NCCc1ccc(O)c(O)c1)c1nnc(C)n1-2] | ✓ | ✓ |
| BRD-N22 | 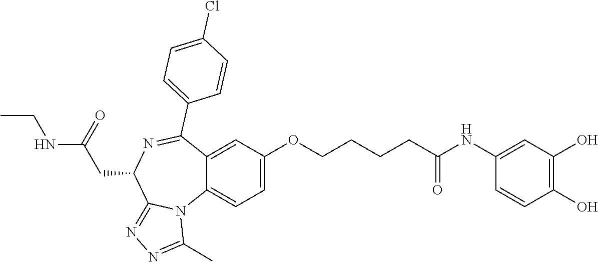 [SMILES: CCNC(=O)C[C@@H]1N=C(c2ccc(Cl)cc2)c2cc(OCCCCC(=O)Nc3ccc(O)c(O)c3)ccc2-n2c(C)nnc21] | ✓ | |
| BRD-N30 | 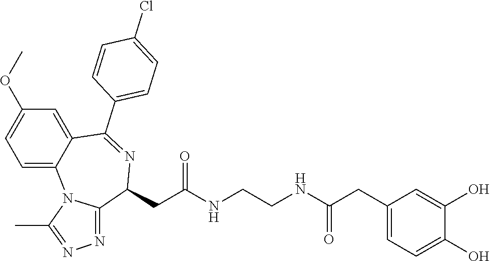 [SMILES: COc1ccc2c(c1)C(c1ccc(Cl)cc1)=N[C@@H](CC(=O)NCCNC(=O)Cc1ccc(O)c(O)c1)c1nnc(C)n1-2] | ✓ | |
| BRD-N38 | 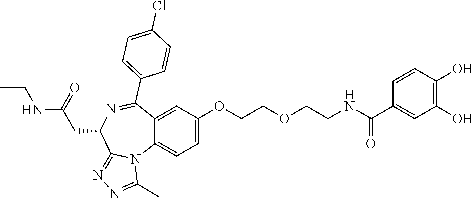 [SMILES: CCNC(=O)C[C@@H]1N=C(c2ccc(Cl)cc2)c2cc(OCCOCCNC(=O)c3ccc(O)c(O)c3)ccc2-n2c(C)nnc21] | ✓ | |
| BRD-N39 | 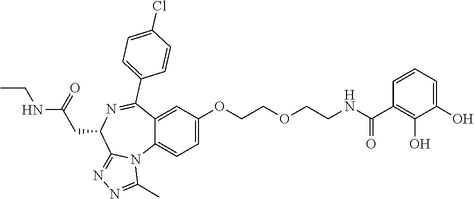 [SMILES: CCNC(=O)C[C@@H]1N=C(c2ccc(Cl)cc2)c2cc(OCCOCCNC(=O)c3cccc(O)c3O)ccc2-n2c(C)nnc21] | ✓ | |
| BRD-N44 | 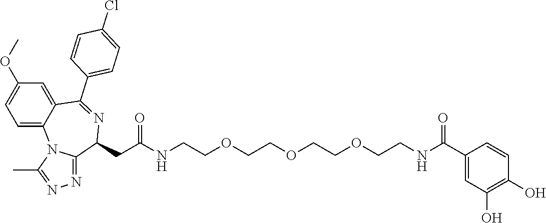 [SMILES: COc1ccc2c(c1)C(c1ccc(Cl)cc1)=N[C@@H](CC(=O)NCCOCCOCCOCCNC(=O)c1ccc(O)c(O)c1)c1nnc(C)n1-2] | ✓ | |
| BRD-N67 | 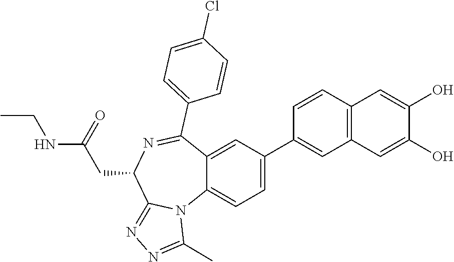 [SMILES: CCNC(=O)C[C@@H]1N=C(c2ccc(Cl)cc2)c2cc(-c3ccc4cc(O)c(O)cc4c3)ccc2-n2c(C)nnc21] | ✓ | ✓ |
| BRD-N68 | 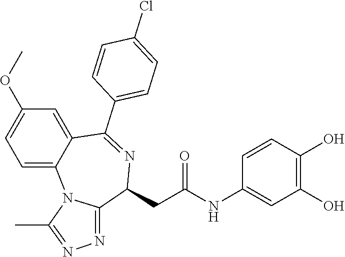 [SMILES: COc1ccc2c(c1)C(c1ccc(Cl)cc1)=N[C@@H](CC(=O)Nc1ccc(O)c(O)c1)c1nnc(C)n1-2] | ✓ | |
| BRD-N69 | 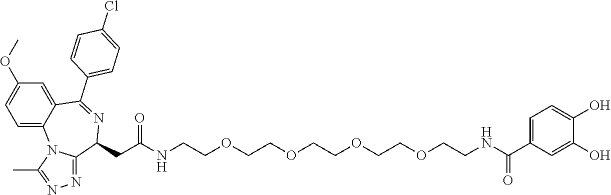 [SMILES: COc1ccc2c(c1)C(c1ccc(Cl)cc1)=N[C@@H](CC(=O)NCCOCCOCCOCCOCCNC(=O)c1ccc(O)c(O)c1)c1nnc(C)n1-2] | ✓ | |
| BRD-N70 | 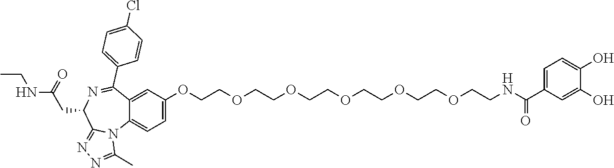 [SMILES: CCNC(=O)C[C@@H]1N=C(c2ccc(Cl)cc2)c2cc(OCCOCCOCCOCCOCCOCCNC(=O)c3ccc(O)c(O)c3)ccc2-n2c(C)nnc21] | ✓ | |
| BRD-N71 | 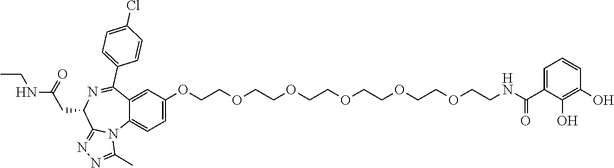 [SMILES: CCNC(=O)C[C@@H]1N=C(c2ccc(Cl)cc2)c2cc(OCCOCCOCCOCCOCCOCCNC(=O)c3cccc(O)c3O)ccc2-n2c(C)nnc21] | ✓ | |

TABLE 5
Summary of Degradation of BRD4 Using a Combination of an Aromatic 1,2-diol-containing BRD4 Ligand and an Aryl-boronic Acid-containing MDM2 Ligand.

| LIGAND | | 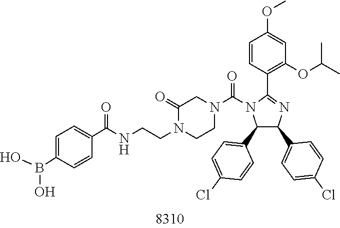 [SMILES: COc1ccc(C2=N[C@@H](c3ccc(Cl)cc3)[C@@H](c3ccc(Cl)cc3)N2C(=O)N2CCN(CCNC(=O)c3ccc(B(O)O)cc3)C(=O)C2)c(OC(C)C)c1] | 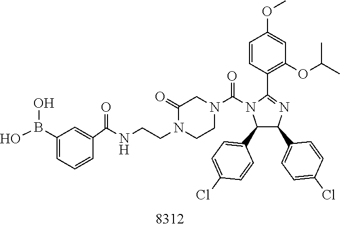 [SMILES: COc1ccc(C2=N[C@@H](c3ccc(Cl)cc3)[C@@H](c3ccc(Cl)cc3)N2C(=O)N2CCN(CCNC(=O)c3cccc(B(O)O)c3)C(=O)C2)c(OC(C)C)c1] |
|---|---|---|---|
| BRD-N10 | 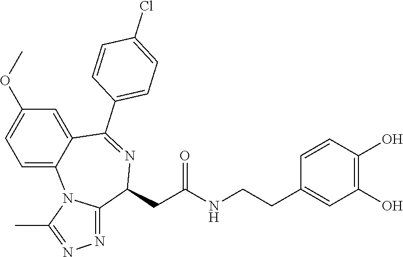 [SMILES: COc1ccc2c(c1)C(c1ccc(Cl)cc1)=N[C@@H](CC(=O)NCCc1ccc(O)c(O)c1)c1nnc(C)n1-2] | ✓ | |
| BRD-N25 | 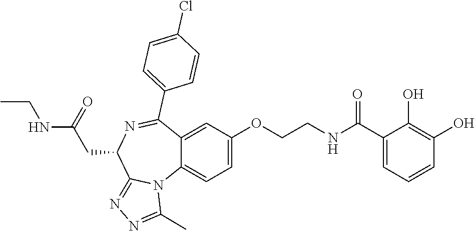 [SMILES: CCNC(=O)C[C@@H]1N=C(c2ccc(Cl)cc2)c2cc(OCCNC(=O)c3cccc(O)c3O)ccc2-n2c(C)nnc21] | ✓ | ✓ |
| BRD-N39 | 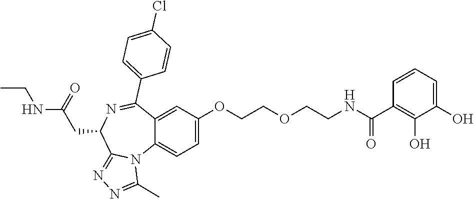 [SMILES: CCNC(=O)C[C@@H]1N=C(c2ccc(Cl)cc2)c2cc(OCCOCCNC(=O)c3cccc(O)c3O)ccc2-n2c(C)nnc21] | ✓ | ✓ |
| BRD-N71 | 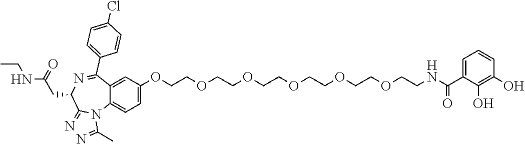 [SMILES: CCNC(=O)C[C@@H]1N=C(c2ccc(Cl)cc2)c2cc(OCCOCCOCCOCCOCCOCCNC(=O)c3cccc(O)c3O)ccc2-n2c(C)nnc21] | | ✓ |

TABLE 6
Summary of Degradation of BRD4 Using a Combination of an Aromatic 1,2-diol-containing BRD4 Ligand and an Aryl-boronic Acid-containing VHL Ligand.

| LIGAND | | 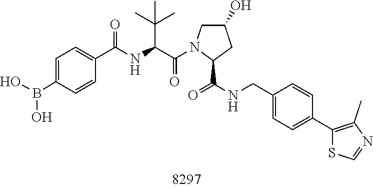 [SMILES: Cc1ncsc1-c1ccc(CNC(=O)[C@@H]2C[C@@H](O)CN2C(=O)[C@@H](NC(=O)c2ccc(B(O)O)cc2)C(C)(C)C)cc1] | 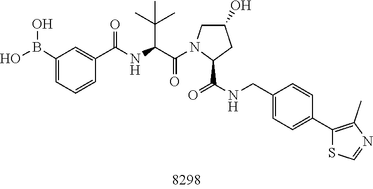 [SMILES: Cc1ncsc1-c1ccc(CNC(=O)[C@@H]2C[C@@H](O)CN2C(=O)[C@@H](NC(=O)c2cccc(B(O)O)c2)C(C)(C)C)cc1] |
|---|---|---|---|
| BRD- N40 | 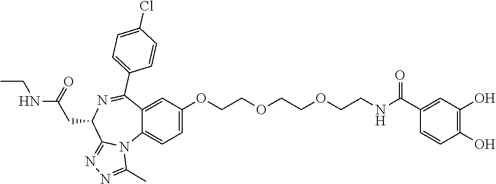 [SMILES: CCNC(=O)C[C@@H]1N=C(c2ccc(Cl)cc2)c2cc(OCCOCCOCCNC(=O)c3ccc(O)c(O)c3)ccc2-n2c(C)nnc21] | ✓ | |

The aforementioned embodiments as well as the examples above highlight a number of advantages of CURE-PRO molecules over either PROTACs or traditional drugs. These advantages include but are not limited to: (i) the combinatorial nature of CURE-PROs significantly reduces synthesis time and effort to identify the optimal E3 ligase (machinery) to target matchup—just 20 ligands to each (set) provides 400 different combinations; (ii) CURE-PROs are half the size of PROTACs, allowing for faster optimization of their PK, solubility, tissue distribution and cellular permeability, ease of oral bioavailability, and ability to cross the blood-brain barrier (BBB); (iii) CURE-PROs allow for the adjustment of individual concentration of the target pharmacophore(s) and the E3 ligase (machinery) ligand to maximize target degradation, while not interfering with the degradation of natural targets of the recruited E3 ligases that the CURE-PROs overcome the “hook-effect”, which severely limits PROTACS; (iv) CURE-PROs enable the degradation of targets where the target-directed pharmacophore binds with average to poor (micro-molar) affinity, while still maintaining high specificity; (v) CURE-PROs enable the preferential degradation of targets aggregates, where use of two or more target-directed pharmacophores provides high specificity, while leaving monomeric native-state protein intact; (vi) CURE-PROs enable the preferential degradation of (aberrantly) modified targets, such as occurs in constitutively signaling oncogene proteins in cancer cells, while providing high specificity to preserve unmodified protein in (non-cancer) normal cells; (vii) CURE-PROs may be designed to recruit transporters to facilitate selective uptake of one or both ligands in target cells and orthogonal cellular uptake mechanisms and the tumor micro-environment may be exploited to concentrate both CURE-PRO partners into target cancer cells; (viii) should CURE-PROs cause unanticipated side-effects in a subset of patients, such side-effects can be rapidly and completely reversed (e.g., ingestion of Epigallocatechin gallate (EGCG) or other Polyphenol compounds) a substrate for covalently linking to CURE-PRO molecules comprising a boronate or phenyl-boronate, will deplete CURE-PRO molecules from their target cells and ultimately lead to their excretion, with this unique feature of CURE-PROs not being accomplished by monoclonal antibodies, PROTACs, or the vast majority of traditional drugs; (ix) since specific E3 ligases can target many proteins, one CURE-PRO ligand may be designed for a given E3 ligase and may be used in conjunction with many partner CURE-PRO pharmacophores to consign many different protein targets for degradation; (x) the CURE-PRO approach allows decreased demands upon monomeric potency and ligand activity, increased flexibility to tune drug properties, and the ability to permeate cells to reach intracellular targets; (xi) the modular design of the CURE-PRO platform allows for structure activity relationships to be explored and the ideal linker length and the preferred E3 ligase or adaptor partner to be identified for specific target degradation—very rapidly by exploiting the combinatorial principles; and (xii) since CURE-PROs need only bind with sufficient affinity to bring the target protein in proximity to a partner E3 ligase, multiple different binding partners may be identified for a given target.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

1. A therapeutic composition comprising:
a first precursor compound having the chemical structure:
E3ULB-C₁-L₁,
or a pharmaceutically acceptable salt, enantiomer, stereoisomer, solvate, or polymorph thereof, and
a second precursor compound having the chemical structure:
TPB-C₂-L₂,
or a pharmaceutically acceptable salt, enantiomer, stereoisomer, solvate, or polymorph thereof, wherein:
E3ULB is a small molecule E3 ubiquitin ligase binding moiety that binds an E3 ubiquitin ligase, an E3 ubiquitin ligase complex, or subunit thereof,
TPB is a small molecule comprising a BET domain protein binding moiety,
C₁ and C₂ are independently a bond or a connector element,
L₁ and L₂ are linker element pairs suitable for binding to one another by two or more reversible covalent bonds that form under physiological conditions, each linker element having a molecular weight of 54 to 420 Daltons, said linker element pairs being selected from the group consisting of:
(1) one linker element comprising an aromatic 1,2-diol-containing moiety and the other linker element comprising an aromatic or heteroaromatic boronic acid- or boronic ester-containing moiety;
(2) one linker element comprising an aromatic 1,2-carbonyl and alcohol-containing moiety and the other linker element comprising an aromatic or heteroaromatic boronic acid- or boronic ester-containing moiety;
(3) one linker element comprising a cis-dihydroxycoumarin-containing moiety and the other linker element comprising an aromatic or heteroaromatic boronic acid- or boronic ester-containing moiety;
(4) one linker element comprising an α-hydroxycarboxylic acid-containing moiety and the other linker element comprising an aromatic or heteroaromatic boronic acid- or boronic ester-containing moiety;
(5) one linker element comprising an aromatic 1,3-diol-containing moiety and the other linker element comprising an aromatic or heteroaromatic boronic acid- or boronic ester-containing moiety;
(6) one linker element comprising an aromatic 2-(aminomethyl)phenol-containing moiety and the other linker element comprising an aromatic or heteroaromatic boronic acid- or boronic ester- or 1,2-boronic acid and carbonyl-containing moiety;
(7) one linker element comprising a cis-1,2-diol-, or cis-1,3-diol-, or a ring system comprising a trans-1,2-diol-containing moiety and the other linker element comprising an aromatic or heteroaromatic boronic acid- or boronic ester-containing moiety;
(8) one linker element comprising a [2.2.1] bicyclic ring system comprising a cis-1,2-diol-, or a cis-1,2-diol and cis-1,3-diol-, or a cis-1,2-diol and a β-hydroxyketone-containing moiety and the other linker element comprising an aromatic or heteroaromatic boronic acid- or boronic ester-containing moiety;
(9) one linker element comprising a [2.2.1] bicyclic ring system comprising a cis-1,2-diol and cis-1,2-aminoalcohol-, or a cis-1,2-diol and cis-1,3-aminoalcohol-, or a cis-1,2-diol and cis-1,2-hydrazine-alcohol-containing moiety and the other linker element comprising an aromatic or heteroaromatic boronic acid- or 1,2-boronic acid and carbonyl-containing moiety;
(10) one linker element comprising a [2.2.1] bicyclic ring system comprising a cis-1,2-aminoalcohol and cis-1,3-diol- or a cis-1,2-aminoalcohol and a p-hydroxyketone-containing moiety and the other linker element comprising an aromatic or heteroaromatic boronic acid- or 1,2-boronic acid and carbonyl-containing moiety;
(11) one linker element comprising a cis-1,2-aminoalcohol-, or a ring system comprising a trans-1,2-aminoalcohol-containing an aromatic or heteroaromatic boronic acid- or boronic ester- or 1,2-boronic acid and carbonyl-containing moiety;
(12) one linker element comprising a cis-1,3-aminoalcohol-containing moiety and the other linker element comprising an aromatic or heteroaromatic boronic acid- or boronic ester- or 1,2-boronic acid and carbonyl-containing moiety;
(13) one linker element comprising an acyl or aromatic hydrazine-containing moiety and the other linker molecule comprising an aromatic or heteroaromatic 1,2-boronic acid and carbonyl-containing moiety; and
(14) one linker element comprising an α-hydroxyketone-containing moiety and the other linker molecule comprising an α-hydroxyketone-containing moiety.

2. The therapeutic composition of claim 1, wherein one of the linker elements L₁ or L₂ is derived from an aromatic 1,2-diol-containing compound comprising the following structure, or salt, enantiomer, stereoisomer, or polymorph thereof: 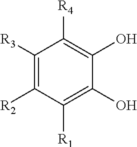
wherein
R₁ to R₄ are independently —H, —OH, —C₁₋₆ alkyl, —C₁₋₆ alkoxy, alkyl amine, —C(O)NH₂, —CN, aryl, heteroaryl, an electron donating moiety, or a bond to —C₁-E3ULB or —C₂-TPB; wherein when two of R₁ to R₄ are adjacent they may optionally be taken together to form one or more fused 5- or 6-membered aromatic, heteroaromatic, carbocyclic, or heterocyclic rings; and wherein one of R₁ to R₄ comprises a bond to —C₁-E3ULB or —C₂-TPB;
and the other linker element, L₂ or L₁ respectively, is derived from an aromatic or heteroaromatic boronic acid- or boronic ester-containing compound is comprised of one of the following structures, or salts, enantiomers, stereoisomers, or polymorphs thereof: 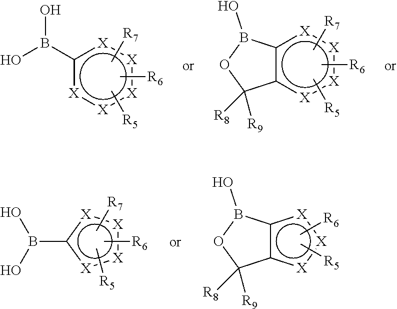
wherein
R₅ to R₇ are independently —H, -halogen, —CF₃, —NO₂, —CN, —OCH₃, —CH₂OH, —C₁₋₆ alkyl, —C₁₋₆ alkoxy, aryl, heteroaryl, —C(O)CH₃, —C(O)CH₂CH₃, or a bond to —C₁-E3ULB or —C₂-TPB;
R₈ and R₉ are independently —H, —C₁₋₆ alkyl, aryl, heteroaryl, a bond to —C₁-E3ULB or —C₂-TPB, or can be connected to each other via a spiro 3-, 4-, 5-, or 6-membered ring;
X is independently C, N, O, or S; and
wherein when two of R₅ to R₇ are adjacent they may optionally be taken together to form one or more fused 5- or 6-membered aromatic, heteroaromatic, carbocyclic, or heterocyclic rings; and one of R₅ to R₇ comprises a bond to —C₂-TPB, or one of R₅ to R₇ comprises a bond to —C₁-E3ULB.

3. The therapeutic composition of claim 2, wherein one of the linker elements L₁ or L₂ is comprised of one of the following structures, or salts, enantiomers, stereoisomers, or polymorphs thereof: 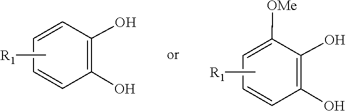
wherein
R₁ is a bond to either —C₁-E3ULB or —C₂-TPB;
and the other linker element, L₂ or L₁, respectively, is comprised of one of the following structures, or salts, enantiomers, stereoisomers, or polymorphs thereof: 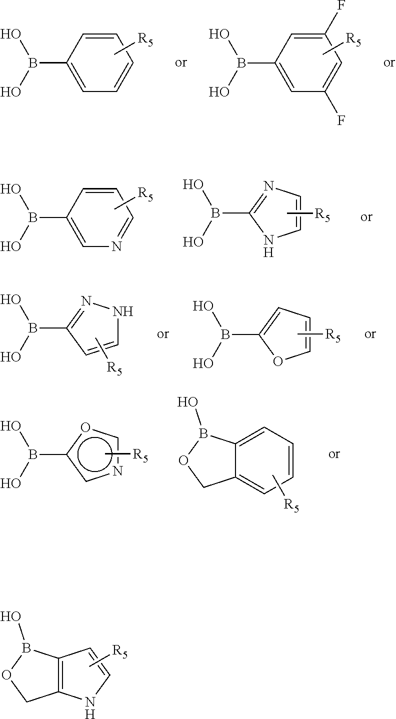
wherein
R₅ is a bond to either —C₁-E3ULB or —C₂-TPB.

4.-29. (canceled)

30. The therapeutic composition of claim 1, wherein the connector element C₁ and/or C₂ comprises the following structure, or salt, enantiomer, stereoisomer, or polymorph thereof: 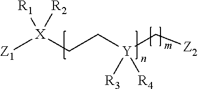
wherein
n and m are independently integers from 0 to 6;
X and Y are independently O, N, C, S, Si, P, or B;
R₁ to R₄ can independently be —H, —OH, —C₁₋₆ alkyl, —C₁₋₆ alkoxy, alkyl amine, aryl, heteroaryl, or —C(O)NH₂; and
Z₁ and Z₂ are independently a bond to E3ULB, -TPB, -L₁ or -L₂; wherein when Z₁ is a bond to -E3ULB or -TPB, Z₂ is a bond to -L₁, or -L₂; and wherein when Z₁ is a bond to -L₁, or -L₂, Z₂ is a bond to E3ULB or -TPB.

31. The therapeutic composition of claim 30, wherein the connector element C₁ and/or C₂ is comprised of one of the following structures, or salts, enantiomers, stereoisomers, or polymorphs thereof: 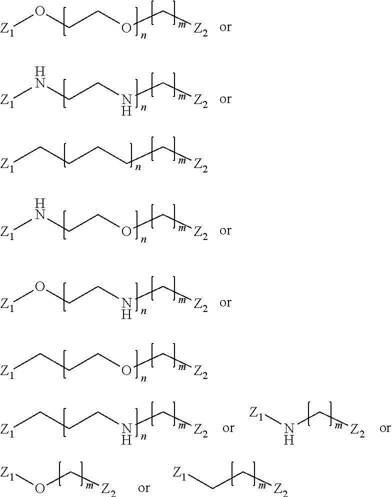
wherein
n and m are independently integers from 0 to 6; and
Z₁ and Z₂ are independently a bond to -E3ULB, -TPB, -L₁ or -L₂; wherein when Z₁ is a bond to -E3ULB or -TPB, Z₂ is a bond to -L₁, or -L₂; and wherein when Z₁ is a bond to -L₁, or -L₂, Z₂ is a bond to -E3ULB or -TPB.

32.-39. (canceled)

40. The therapeutic composition of claim 1, wherein the TPB is a BET domain protein binding moiety, and has the following structure, or salts, enantiomers, stereoisomers, or polymorphs thereof: 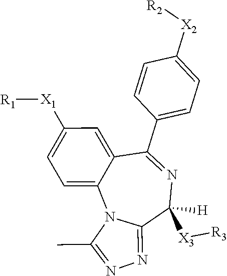
wherein
X₁ to X₃ are independently C, O, N, S, B, F, Cl, or Br;
R₁ to R₃ are independently a lone pair of electrons, —H, —C₁₋₆ alkyl, —C₁₋₆ alkoxy, alkyl amine, aryl, heteroaryl, —C₁₋₄ ester, —C(O)OH, an amide, or a bond to —C₂-L₂; and wherein one of R₁ to R₃ comprises a bond to —C₂-L₂.

41. The therapeutic composition of claim 40, wherein the BET domain protein binding moiety has the following structure, or salts, enantiomers, stereoisomers, or polymorphs thereof: 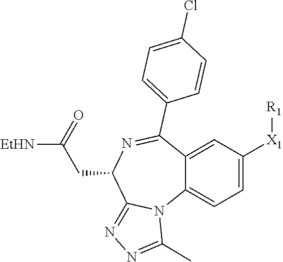
wherein
X₁ is C, O, N, S, or B; and
R₁ comprises a bond to —C₂-L_{2;}
and the E3ULB-C₁-L₁ first precursor compound is one of the following structures, or salts, enantiomers, stereoisomers, or polymorphs thereof:

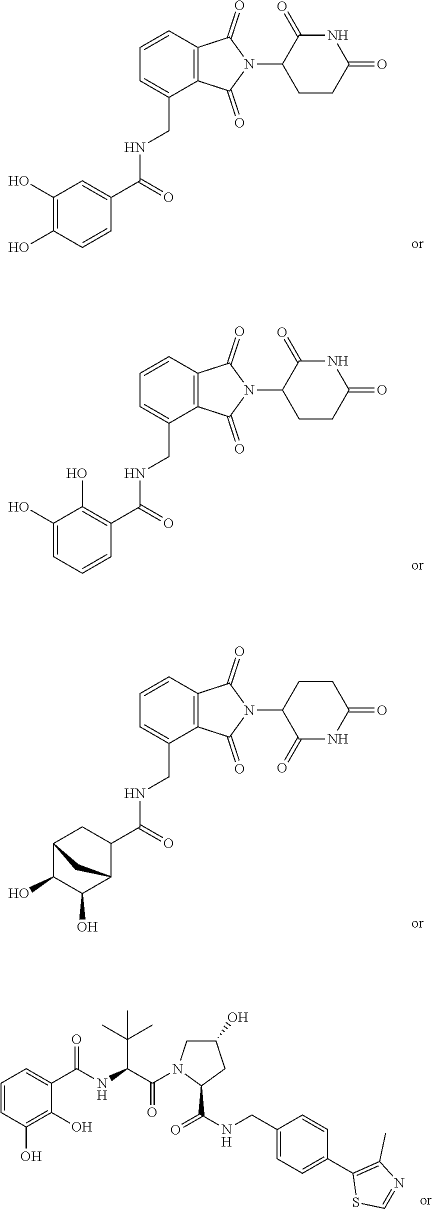 [SMILES: O=C1CCC(N2C(=O)c3cccc(CNC(=O)c4ccc(O)c(O)c4)c3C2=O)C(=O)N1 ; O=C1CCC(N2C(=O)c3cccc(CNC(=O)c4cccc(O)c4O)c3C2=O)C(=O)N1 ; O=C1CCC(N2C(=O)c3cccc(CNC(=O)C4C[C@@H]5C[C@H]4[C@@H](O)[C@H]5O)c3C2=O)C(=O)N1 ; Cc1ncsc1-c1ccc(CNC(=O)[C@@H]2C[C@@H](O)CN2C(=O)[C@@H](NC(=O)c2cccc(O)c2O)C(C)(C)C)cc1]

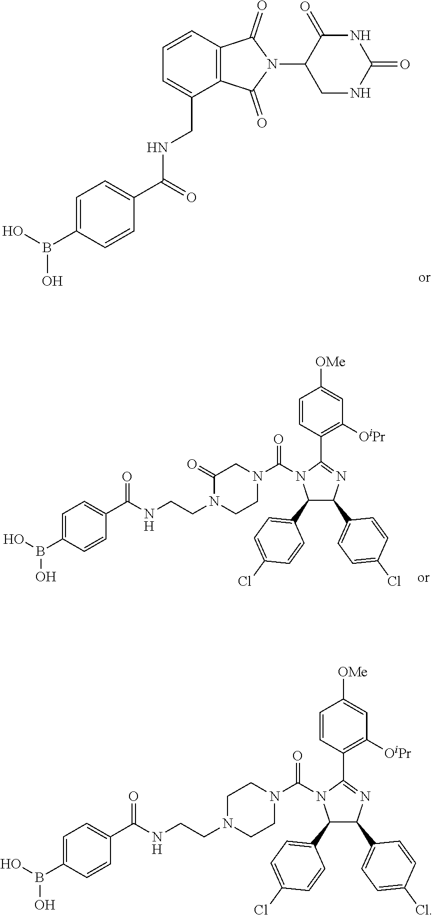 [SMILES: O=C1NCC(N2C(=O)c3cccc(CNC(=O)c4ccc(B(O)O)cc4)c3C2=O)C(=O)N1 ; COc1ccc(C2=N[C@@H](c3ccc(Cl)cc3)[C@@H](c3ccc(Cl)cc3)N2C(=O)N2CCN(CCNC(=O)c3ccc(B(O)O)cc3)C(=O)C2)c(OC(C)C)c1 ; COc1ccc(C2=N[C@@H](c3ccc(Cl)cc3)[C@@H](c3ccc(Cl)cc3)N2C(=O)N2CCN(CCNC(=O)c3ccc(B(O)O)cc3)CC2)c(OC(C)C)c1]

42. The therapeutic composition of claim 41, wherein the BET domain protein binding moiety-containing second precursor compound is one of the following structures, or salts, enantiomers, stereoisomers, or polymorphs thereof:

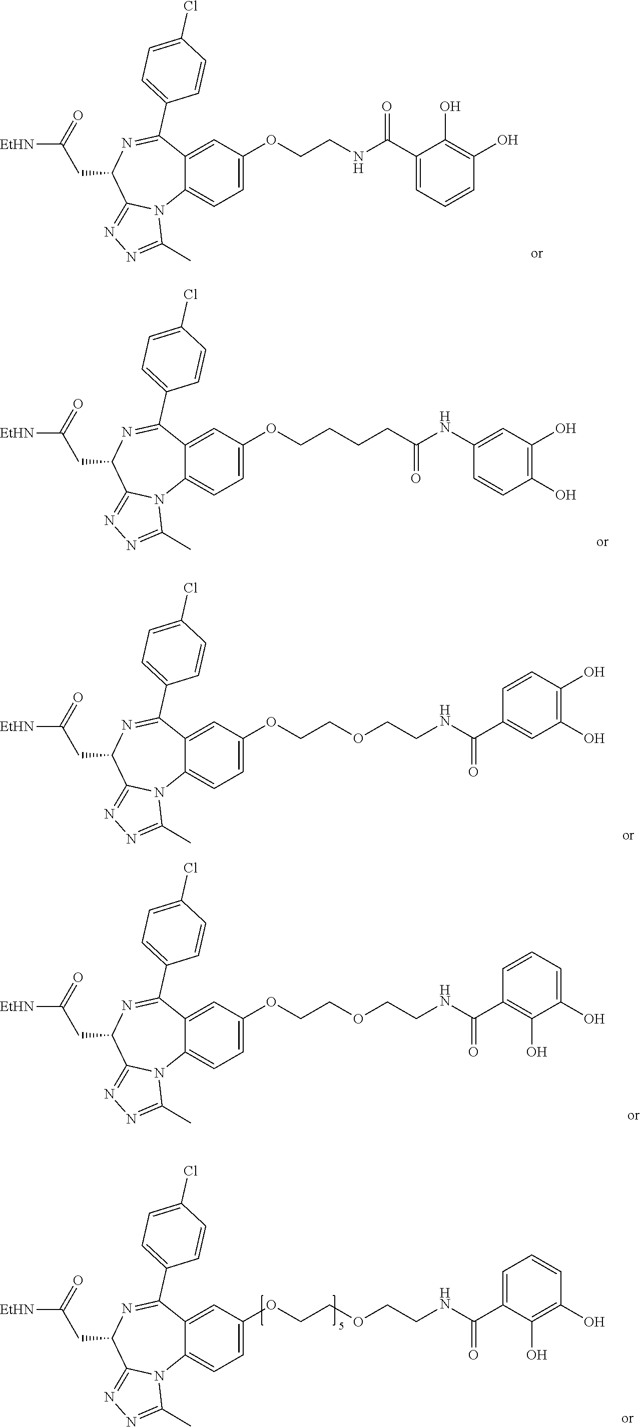

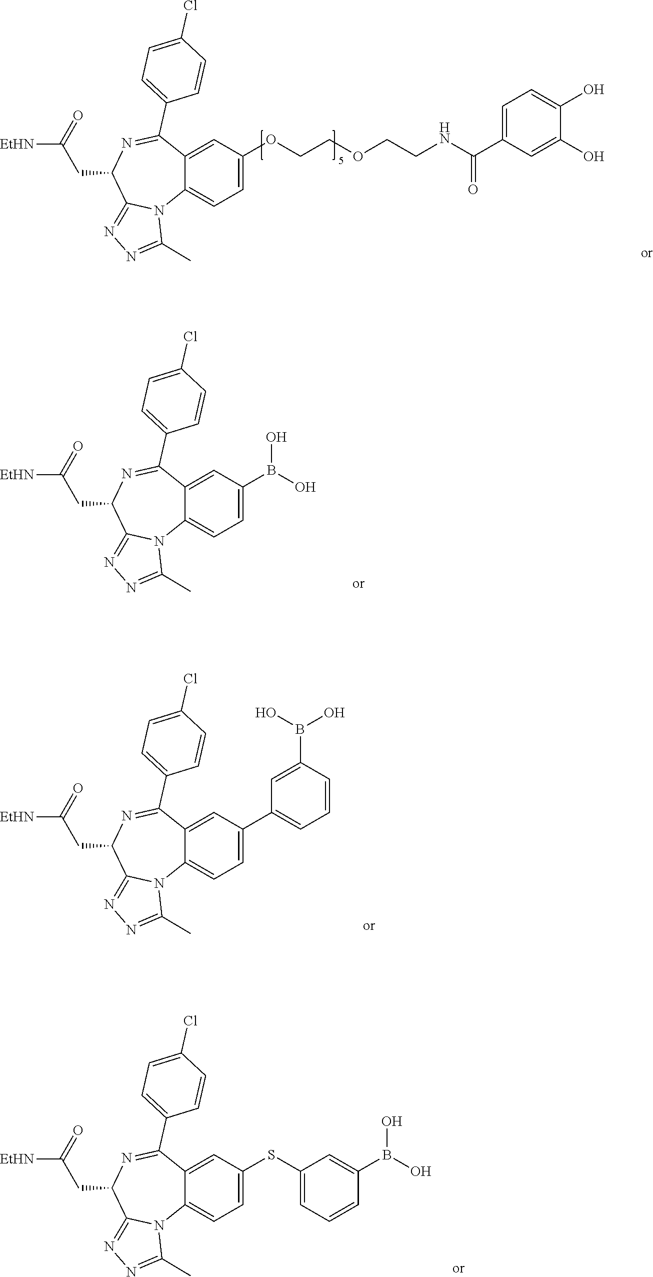

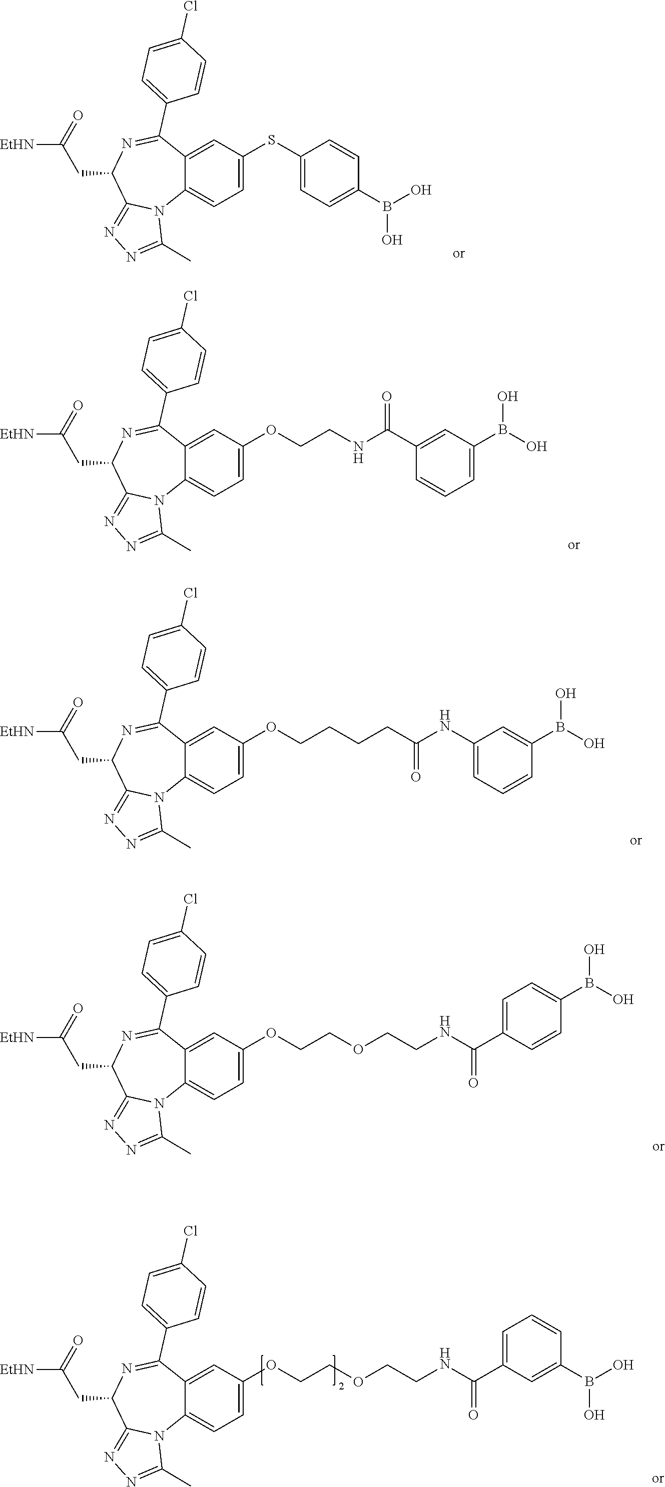

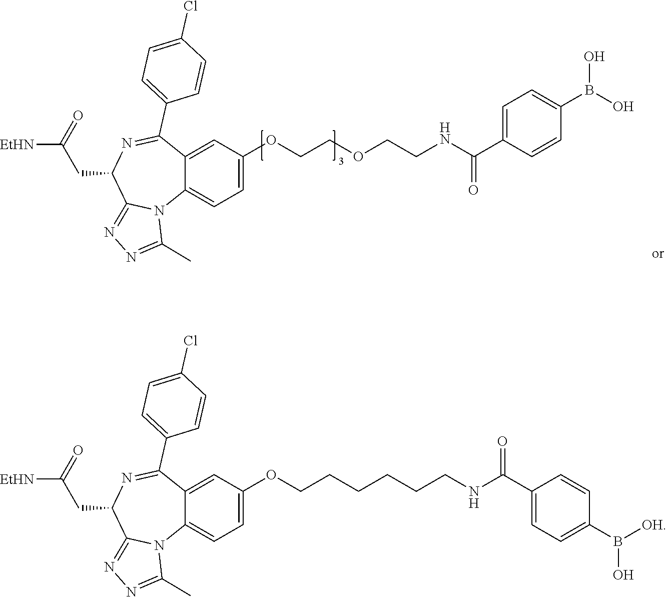

43. The therapeutic composition of claim 40, wherein the BET domain protein binding moiety has the following structure, or salts, enantiomers, stereoisomers, or polymorphs thereof: 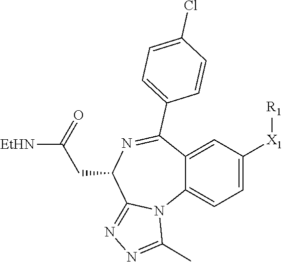
wherein
X₁ is C, O, N, S, or B;
R₁ comprises a bond to —C₂-L₂;
the linker element L₂ is comprised of an aromatic or heteroaromatic boronic acid- or boronic ester-containing moiety; and
the E3ULB-C₁-L₁ first precursor compound is one of the following structures, or salts, enantiomers, stereoisomers, or polymorphs thereof: 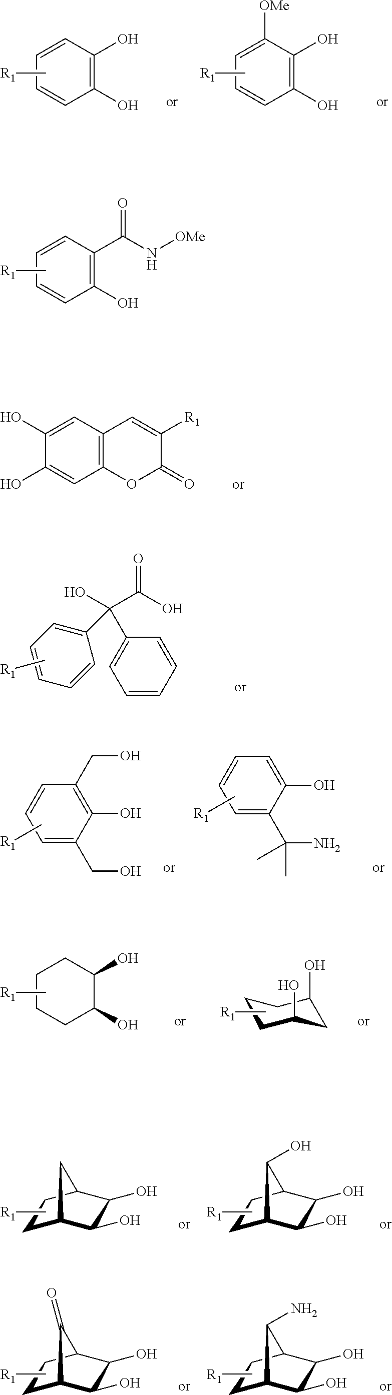 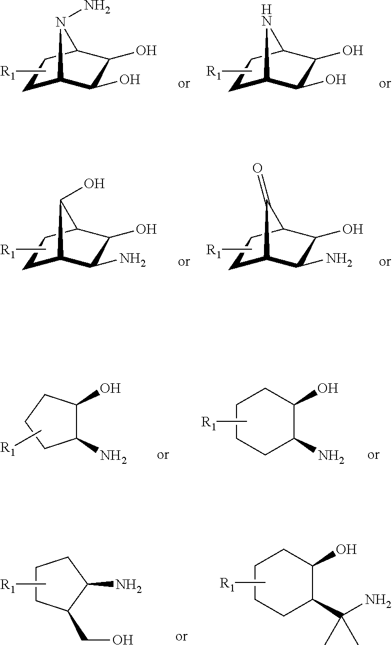
wherein
R₁ is —C₁-E3ULB.

44.-46. (canceled)

47. The therapeutic composition of claim 40, wherein the BET domain protein binding moiety has the following structure, or salts, enantiomers, stereoisomers, or polymorphs thereof: 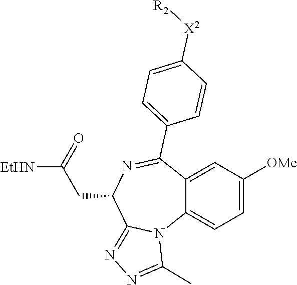
wherein
X₂ is C, O, N, S, or B; and
R₂ comprises a bond to —C₂-L₂;
and the E3ULB-C₁-L₁ moiety-containing first precursor compound is comprised of one of the following structures, or salts, enantiomers, stereoisomers, or polymorphs thereof:

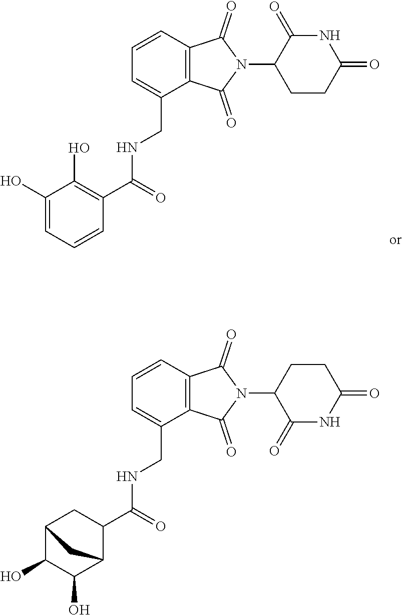 [SMILES: O=C1CCC(N2C(=O)c3cccc(CNC(=O)c4cccc(O)c4O)c3C2=O)C(=O)N1 ; O=C1CCC(N2C(=O)c3cccc(CNC(=O)C4C[C@@H]5C[C@H]4[C@@H](O)[C@H]5O)c3C2=O)C(=O)N1]

48. The therapeutic composition of claim 47, wherein the BET domain protein binding moiety-containing second precursor compound has the following structure, or salts, enantiomers, stereoisomers, or polymorphs thereof:

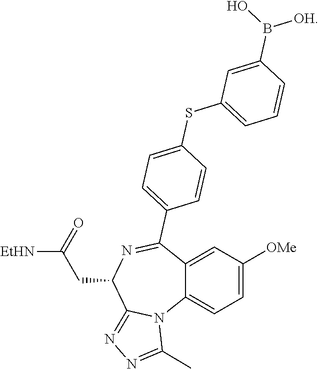

49. The therapeutic composition of claim 40, wherein the BET domain protein binding moiety has the following structure, or salts, enantiomers, stereoisomers, or polymorphs thereof: 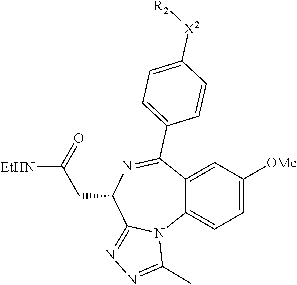
wherein
X₂ is C, O, N, S, or B; and
R₂ comprises a bond to —C₂-L₂;
the linker element L₂ is comprised of an aromatic or heteroaromatic boronic acid- or boronic ester-containing moiety; and
the E3ULB-C₁-L₁ first precursor compound is one of the following structures, or salts, enantiomers, stereoisomers, or polymorphs thereof: 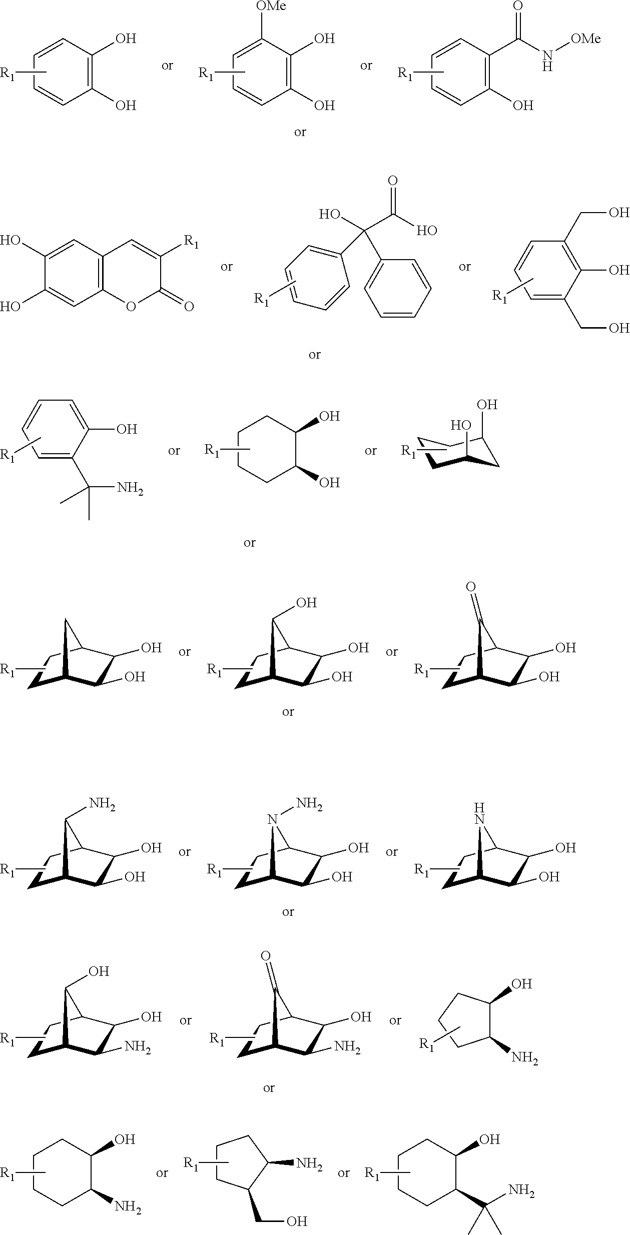
wherein
R₁ is —C₁-E3ULB.

50.-52. (canceled)

53. The therapeutic composition of claim 40, wherein the BET domain protein binding moiety has the following structure, or salts, enantiomers, stereoisomers, or polymorphs thereof: 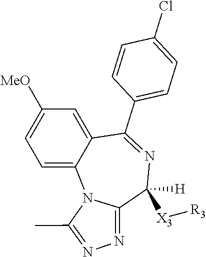
wherein
X₃ is C, O, N, S, or B; and
R₃ comprises a bond to —C₂-L₂;
the linker element L₂ is comprised of an aromatic or heteroaromatic boronic acid- or boronic ester-containing moiety; and
and the E3ULB-C₁-L₁-containing first precursor compound is one of the following structures, or salts, enantiomers, stereoisomers, or polymorphs thereof:

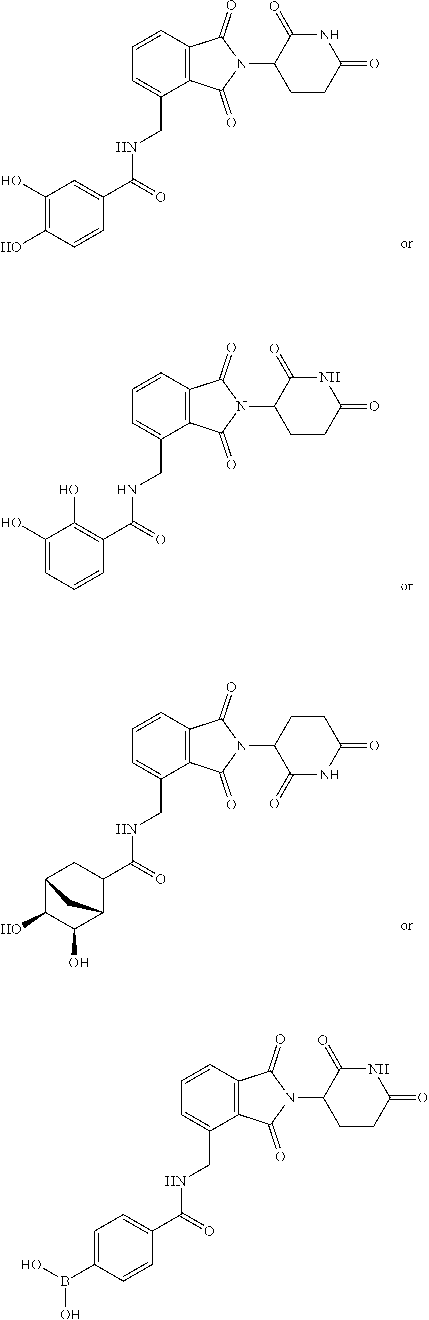 [SMILES: O=C1CCC(N2C(=O)c3cccc(CNC(=O)c4ccc(O)c(O)c4)c3C2=O)C(=O)N1 ; O=C1CCC(N2C(=O)c3cccc(CNC(=O)c4cccc(O)c4O)c3C2=O)C(=O)N1 ; O=C1CCC(N2C(=O)c3cccc(CNC(=O)C4C[C@@H]5C[C@H]4[C@@H](O)[C@H]5O)c3C2=O)C(=O)N1 ; O=C1CCC(N2C(=O)c3cccc(CNC(=O)c4ccc(B(O)O)cc4)c3C2=O)C(=O)N1]

54. The therapeutic composition of claim 53, wherein the BET domain protein binding moiety-containing second precursor compound is one of the following structures, or salts, enantiomers, stereoisomers, or polymorphs thereof:

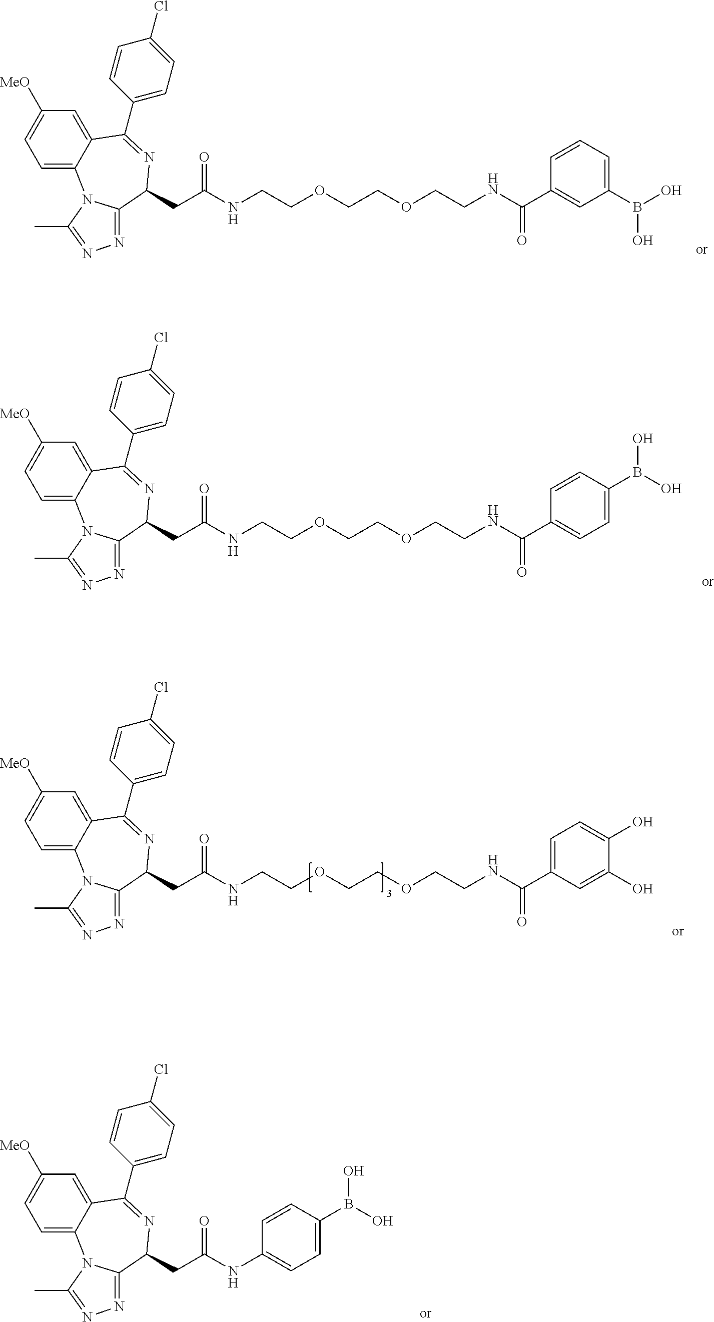

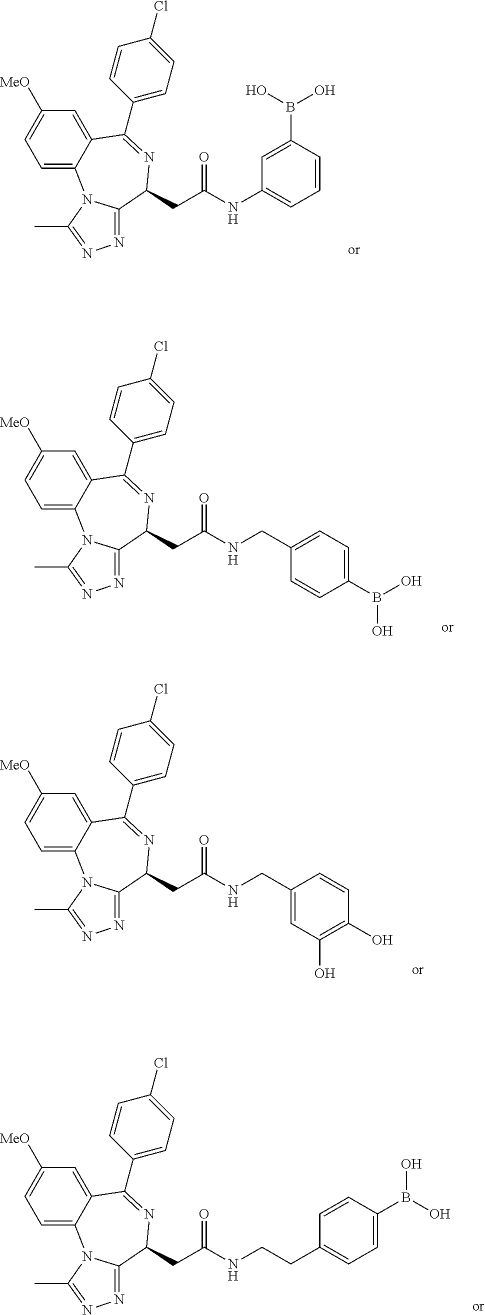 [SMILES: COc1ccc2c(c1)C(c1ccc(Cl)cc1)=N[C@@H](CC(=O)Nc1cccc(B(O)O)c1)c1nnc(C)n1-2 ; COc1ccc2c(c1)C(c1ccc(Cl)cc1)=N[C@@H](CC(=O)NCc1ccc(B(O)O)cc1)c1nnc(C)n1-2 ; COc1ccc2c(c1)C(c1ccc(Cl)cc1)=N[C@@H](CC(=O)NCc1ccc(O)c(O)c1)c1nnc(C)n1-2 ; COc1ccc2c(c1)C(c1ccc(Cl)cc1)=N[C@@H](CC(=O)NCCc1ccc(B(O)O)cc1)c1nnc(C)n1-2]

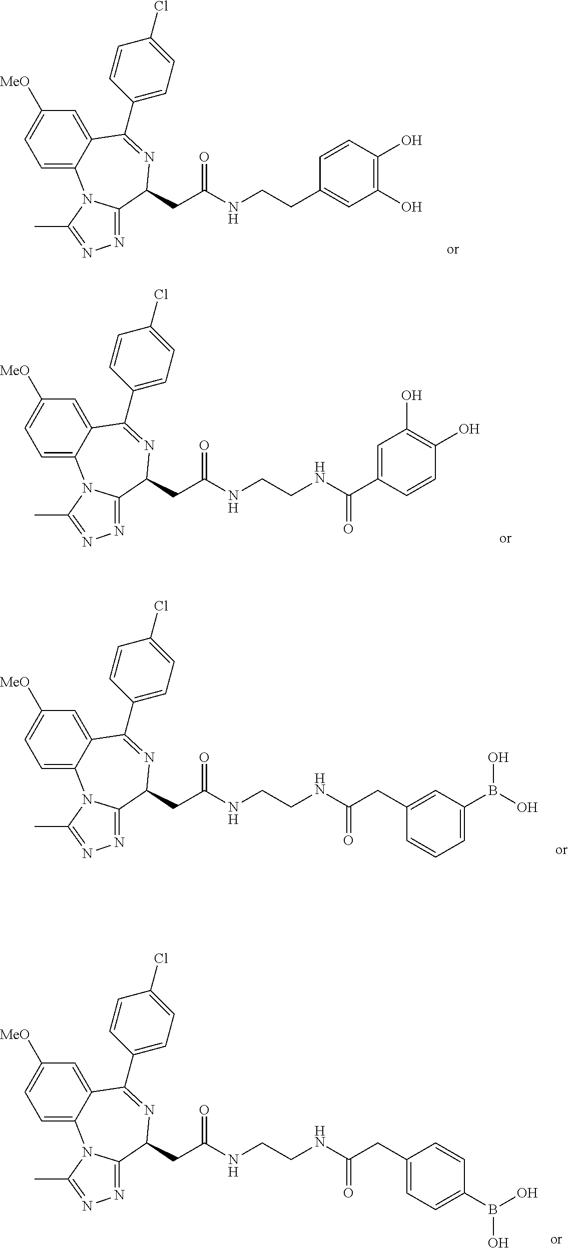 [SMILES: COc1ccc2c(c1)C(c1ccc(Cl)cc1)=N[C@@H](CC(=O)NCCc1ccc(O)c(O)c1)c1nnc(C)n1-2 ; COc1ccc2c(c1)C(c1ccc(Cl)cc1)=N[C@@H](CC(=O)NCCNC(=O)c1ccc(O)c(O)c1)c1nnc(C)n1-2 ; COc1ccc2c(c1)C(c1ccc(Cl)cc1)=N[C@@H](CC(=O)NCCNC(=O)Cc1cccc(B(O)O)c1)c1nnc(C)n1-2 ; COc1ccc2c(c1)C(c1ccc(Cl)cc1)=N[C@@H](CC(=O)NCCNC(=O)Cc1ccc(B(O)O)cc1)c1nnc(C)n1-2]

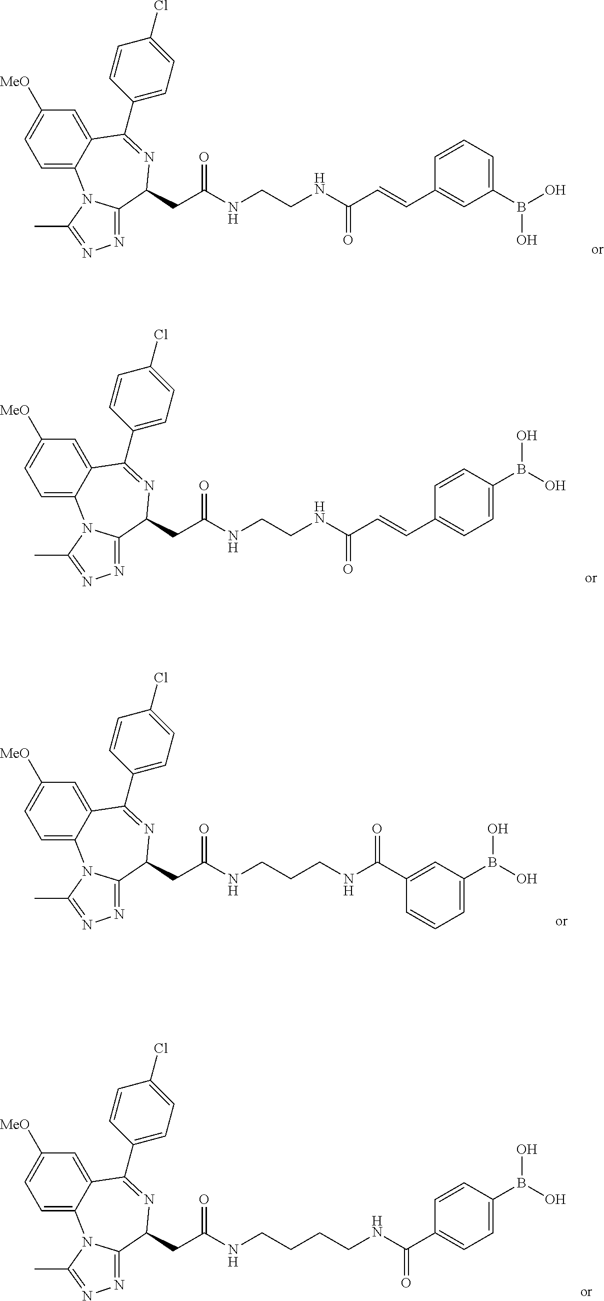 [SMILES: COc1ccc2c(c1)C(c1ccc(Cl)cc1)=N[C@@H](CC(=O)NCCNC(=O)/C=C/c1cccc(B(O)O)c1)c1nnc(C)n1-2 ; COc1ccc2c(c1)C(c1ccc(Cl)cc1)=N[C@@H](CC(=O)NCCNC(=O)/C=C/c1ccc(B(O)O)cc1)c1nnc(C)n1-2 ; COc1ccc2c(c1)C(c1ccc(Cl)cc1)=N[C@@H](CC(=O)NCCCNC(=O)c1cccc(B(O)O)c1)c1nnc(C)n1-2 ; COc1ccc2c(c1)C(c1ccc(Cl)cc1)=N[C@@H](CC(=O)NCCCCNC(=O)c1ccc(B(O)O)cc1)c1nnc(C)n1-2]

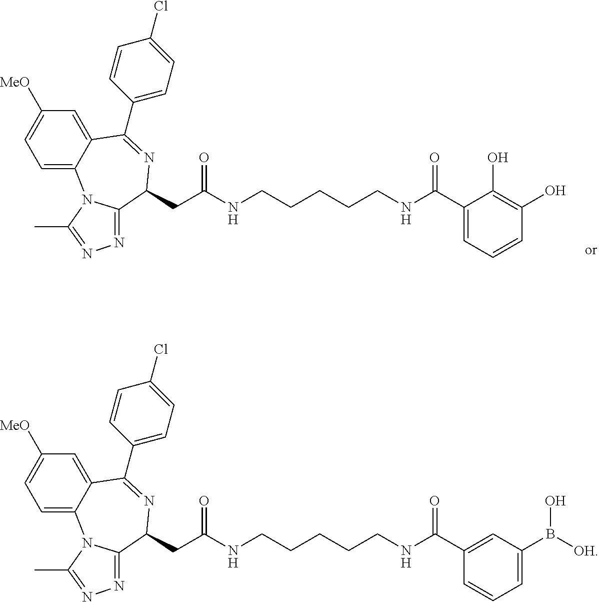 [SMILES: COc1ccc2c(c1)C(c1ccc(Cl)cc1)=N[C@@H](CC(=O)NCCCCCNC(=O)c1cccc(O)c1O)c1nnc(C)n1-2 ; COc1ccc2c(c1)C(c1ccc(Cl)cc1)=N[C@@H](CC(=O)NCCCCCNC(=O)c1cccc(B(O)O)c1)c1nnc(C)n1-2]

55. The therapeutic composition of claim 1, wherein the E3ULB ubiquitin binding moiety binds to the CRBN subunit of the CULLIN4A or CULLIN4B E3 ligase machinery and has the following structure, or salts, enantiomers, stereoisomers, or polymorphs thereof: 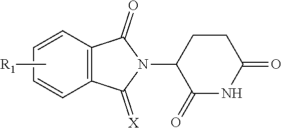
wherein
X is H₂, NH, O, or S; and
R₁ comprises a bond to —C₁-L₁;
or has the following structure, or salts, enantiomers, stereoisomers, or polymorphs thereof: 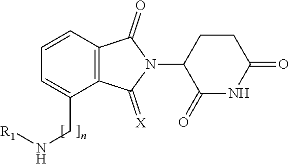
wherein
X is —H₂, —NH, —O, or —S;
n is an integer from 0-10; and
R₁ comprises a bond to —C₁-L₁;
or has the following structure, or salts, enantiomers, stereoisomers, or polymorphs thereof: 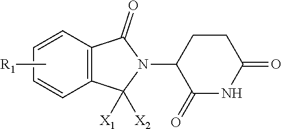
wherein
X₁ and X₂ are independently —H or —C₁₋₆ alkyl; and
R₁ comprises a bond to —C₁-L₁;
or has the following structure, or salts, enantiomers, stereoisomers, or polymorphs thereof: 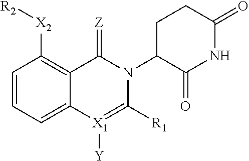
wherein
X₁ and X₂ are independently C, O, N, or S;
R₁ or R₂ are independently —H; C₁₋₆ alkyl, —C₁₋₆ alkoxy, alkyl amine, —C(O)NH₂, or a bond to —C₁-L₁;
Y is a lone pair, —H, —C₁₋₆ alkyl, —C₁₋₆ alkoxy, alkyl amine, —C(O)NH₂, or a bond to —C₁-L₁; and
Z is —H₂, —NH, —O, or —S;
wherein one of R₁, R₂, or Y comprises a bond to —C₁-L₁;
or has the following structure, or salts, enantiomers, stereoisomers, or polymorphs thereof: 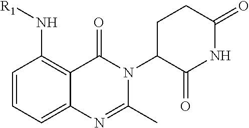
wherein
R₁ comprises a bond to —C₁-L₁.

56.-57. (canceled)

58. The therapeutic composition of claim 1, wherein the E3ULB ubiquitin binding moiety binds to a VHL subunit of the CULLIN2 or CULLIN5 E3 ligase machinery and has the following structure, or salts, enantiomers, stereoisomers, or polymorphs thereof: 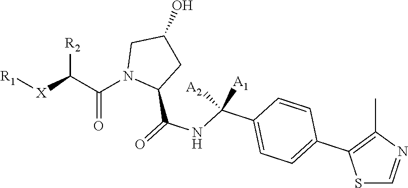
wherein
R₁ to R₂ are independently —H, —C₁₋₆ alkyl, or a bond to —C₁-L₁;
A₁ and A₂ are independently —H, —C₁₋₆ alkyl, —C₁₋₆ alkoxy, alkyl amine, —C(O)NH₂, or a bond to —C₁-L₁; and
X is independently —H, —C₁₋₆ alkyl, heteroalkyl, aryl, heteroaryl, alkyl(aryl), alkyl(heteroaryl), or a natural or unnatural amino acid;
wherein one of R₁, R₂, A₁, or A₂ comprises a bond to —C₁-L₁;
or has the following structure, or salts, enantiomers, stereoisomers, or polymorphs thereof: 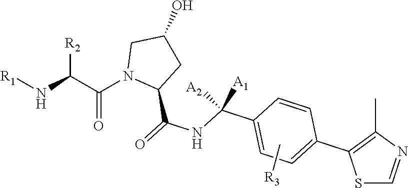
wherein
R₁ to R₃ are independently —H, —C₁₋₆ alkyl, aryl, heteroaryl, —C₁₋₆ alkyl aryl, —C₁₋₆ alkyl(heteroaryl), an amino acid or a bond to —C₁-L₁; and
A₁ and A₂ are independently —H, —C₁₋₆ alkyl, —C₁₋₆ alkoxy, alkyl amine, —CH₂C(O)OH; —CH₂C(O)NH₂, —C(O)NH₂, or a bond to —C₁-L₁;
wherein one of R₁ to R₃, A₁, or A₂ comprises a bond to —C₁-L₁,
or has the following structure, or salts, enantiomers, stereoisomers, or polymorphs thereof: 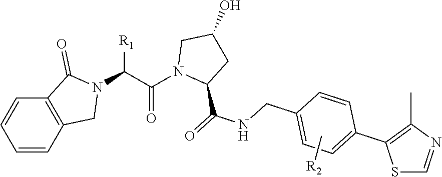
wherein
R₁ to R₂ are independently —H, —C₁₋₆ alkyl, or a bond to —C₁-L₁;
wherein one of R₁ to R₂ comprises a bond to —C₁-L₁;
or has one of the following structures, or salts, enantiomers, stereoisomers, or polymorphs thereof: 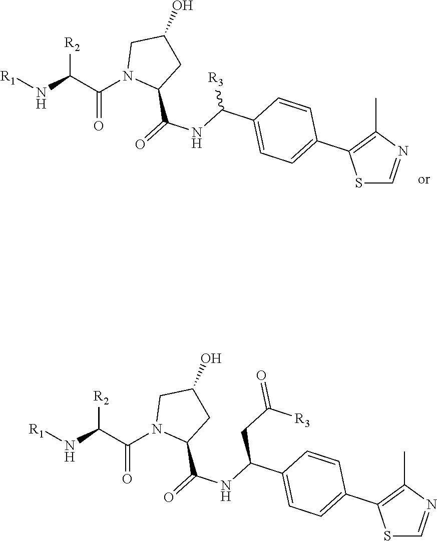
wherein
R₁ to R₃ are independently —H, —C₁₋₆ alkyl, heteroalkyl, aryl, heteroaryl, alkyl(aryl), alkyl(heteroaryl), natural or unnatural amino acid, or a bond to —C₁-L₁; wherein one of R₁ to R₃ comprises a bond to —C₁-L₁.

59. The therapeutic composition of claim 58, wherein the E3ULB ubiquitin binding moiety that binds to the VHL subunit of the CULLIN2 or CULLIN5 E3 ligase machinery has the following structure, or salts, enantiomers, stereoisomers, or polymorphs thereof: 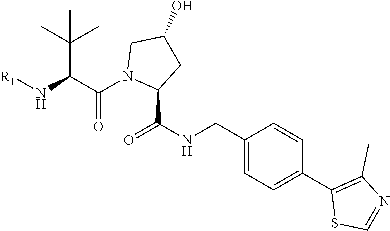
wherein
R₁ comprises a bond to —C₁-L₁.

60. The therapeutic composition of claim 1, wherein the E3ULB ubiquitin binding moiety binds to the MDM2 E3 ligase and has the following structure, or salts, enantiomers, stereoisomers, or polymorphs thereof: 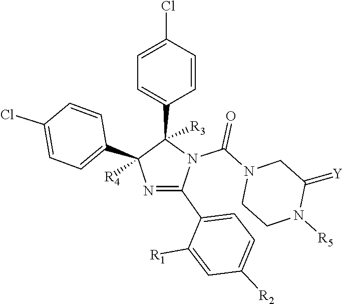
wherein
R₁ to R₅ are independently —H, —OH, —C₁₋₆ alkyl, —C₁₋₆ alkoxy, alkyl amine, aryl, heteroaryl, —C(O)NH₂, or a bond to —C₁-L₁; and
Y is independently H₂ or O;
wherein one of R₁ to R₅ comprises a bond to —C₁-L₁;
or has the following structure, or salts, enantiomers, stereoisomers, or polymorphs thereof: 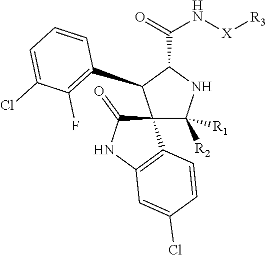
wherein
R₁ to R₃ are independently —H, —OH, —C₁₋₆ alkyl, —C₁₋₆ alkoxy, alkyl amine, aryl, heteroaryl, or a bond to —C₁-L₁; and
X is independently H₂, R₃, a carbocycle, heterocycle, aryl, heteroaryl, -alkyl(aryl), or -alkyl(heteroaryl) group; and
wherein one of R₁ to R₃ comprises a bond to —C₁-L₁;
or has the following structure, or salts, enantiomers, stereoisomers, or polymorphs thereof: 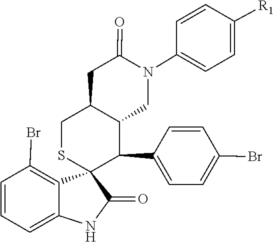
wherein R₁ comprises a bond to —C₁-L₁,
or has the following structure, or salts, enantiomers, stereoisomers, or polymorphs thereof: 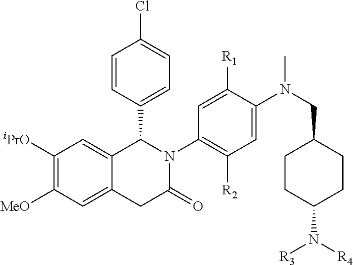
wherein
R₁ to R₄ are independently —H, —OH, —C₁₋₆ alkyl, —C₁₋₆ alkoxy, alkyl amine, aryl, heteroaryl, or a bond to —C₁-L₁, wherein one of R₁ to R₄ comprises a bond to —C₁-L₁;
or comprises the following structure, or salt, enantiomer, stereoisomer, or polymorph thereof: 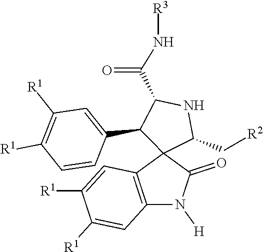
wherein
R₁ are independently —H, —OH, or halogen; and
R² and R³ are independently —H, —C₁₋₆ alkyl, —C₁₋₆ alkoxy, aryl, heteroaryl, alkyl amine, —C(O)NH₂, or a bond to —C₁-L₁, wherein one of R² or R³ comprises a bond to —C₁-L₁;
or comprises the following structure, or salt, enantiomer, stereoisomer, or polymorph thereof: 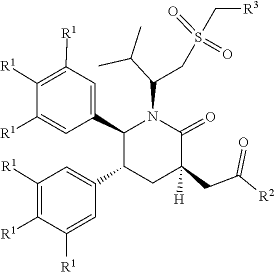
wherein
R¹ are independently —H, —OH, or halogen; and
R² and R³ are independently —H, —C₁₋₆ alkyl, —C₁₋₆ alkoxy, aryl, heteroaryl, alkyl amine, —C(O)NH₂, or a bond to —C₁-L₁, wherein one of R² or R³ comprises a bond to —C₁-L₁;
or comprises the following structure, or salt, enantiomer, stereoisomer, or polymorph thereof: 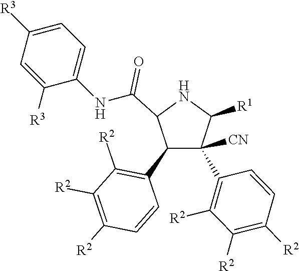
wherein
R² are independently —H, —OH, or halogen; and
R¹ and R³ are independently —H, —C₁₋₆ alkyl, —C₁₋₆ alkoxy, aryl, heteroaryl, alkyl amine, —C(O)NH₂, —COOH, or a bond to —C₁-L₁, wherein one of R or R³ comprises a bond to —C₁-L₁;
or comprises the following structure, or salt, enantiomer, stereoisomer, or polymorph thereof: 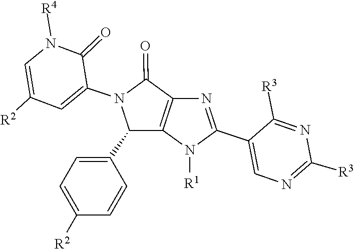
wherein
R² are independently —H, —OH, or halogen; and
R¹, R³ and R⁴ are independently —H, —C₁₋₆ alkyl, —C₁₋₆ alkoxy, aryl, heteroaryl, alkyl amine, —C(O)NH₂, or a bond to —C₁-L₁, wherein one of R¹, R³ or R⁴ comprises a bond to —C₁-L₁,
or comprises the following structure, or salt, enantiomer, stereoisomer, or polymorph thereof: 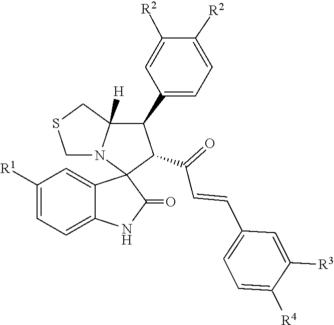
wherein
R¹ is —H, —OH, or halogen; and
R², R³ and R⁴ are independently —H, —C₁₋₆ alkyl, —C₁₋₆ alkoxy, aryl, heteroaryl, halogen, alkyl amine, —C(O)NH₂, or a bond to —C₁-L₁, wherein one of R², R³ or R⁴ comprises a bond to —C₁-L₁;
or comprises the following structure, or salt, enantiomer, stereoisomer, or polymorph thereof: 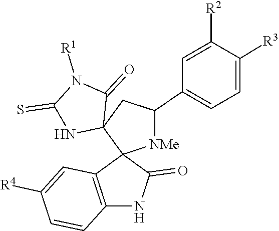
wherein
R¹ is —H, —C₁₋₆ alkyl, —C₁₋₆, aryl, heteroaryl, alkyl amine, —C(O)NH₂, or a bond to —C₁-L₁;
R² and R³ are independently —H, —C₁₋₆ alkyl, —C₁₋₆ alkoxy, aryl, heteroaryl, halogen, alkyl amine, —C(O)NH₂, or a bond to —C₁-L₁; and
R⁴ is —H, —OH, or halogen, wherein one of R¹, R² or R³ comprises a bond to —C₁-L₁;
or comprises the following structure, or salt, enantiomer, stereoisomer, or polymorph thereof: 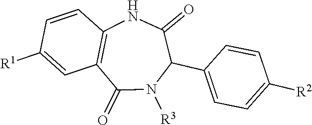
wherein
R¹ and R² are independently —H, —CH₃, —CH₂CH₃, —CH(CH₃)₂, —CF₃, —OCF₃, —OH, —OMe, or halogen; and
R³ is a bond to —C₁-L₁,
or comprises the following structure, or salt, enantiomer, stereoisomer, or polymorph thereof: 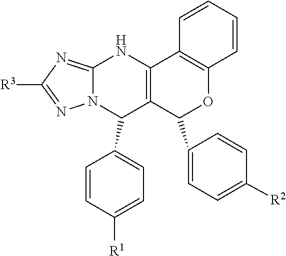
wherein
R¹ and R² are independently —H, —OH, or halogen; and
R³ is a bond to —C₁-L₁.

61.-62. (canceled)

63. The therapeutic composition of claim 1, wherein the E3ULB ubiquitin binding moiety binds to an inhibitor of apoptosis proteins E3 ubiquitin ligase and has the following structure: 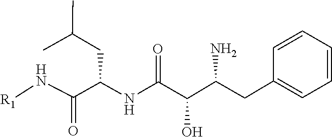
wherein
R₁ comprises a bond to —C₁-L₁;
or has the following structure, or salts, enantiomers, stereoisomers, or polymorphs thereof: 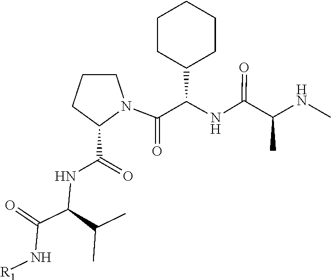
wherein
R₁ comprises a bond to —C₁-L₁;
or has the following structure, or salts, enantiomers, stereoisomers, or polymorphs thereof: 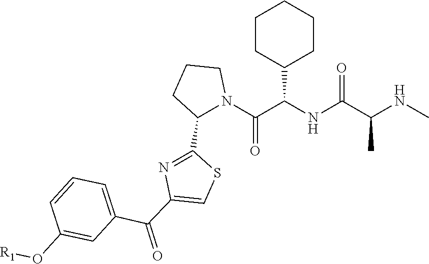
wherein
R₁ comprises a bond to —C₁-L₁.

64.-75. (canceled)

76. The therapeutic composition of claim 1 further comprising:
a third precursor compound having the chemical structure:
E3ULB₂—C₃-L₃,
or a pharmaceutically acceptable salt, enantiomer, stereoisomer, solvate, or polymorph thereof, wherein:
E3ULB₂ is a small molecule E3 ubiquitin ligase binding moiety that binds an E3 ubiquitin ligase, an E3 ubiquitin ligase complex, or subunit thereof that differs in structure from E3ULB,
C₃ is a bond or a connector element, and
L₃ is linker element having a molecular weight of 54 to 420 Daltons and capable of binding to L₂, by two or more reversible bonds that form under physiological conditions, wherein L₂ and L₃ are selected from the group consisting of linker element pairs (1) to (14).

77. The therapeutic composition of claim 1 further comprising:
a third precursor compound having the chemical structure:
TPB₂—C₃-L₃,
or a pharmaceutically acceptable salt, enantiomer, stereoisomer, solvate, or polymorph thereof, wherein:
TPB₂ is a small molecule comprising a BET domain protein binding moiety that differs in structure from TPB,
C₃ is a bond or a connector element, and
L₃ is linker element having a molecular weight of 54 to 420 Daltons and capable of binding to L₁, by two or more reversible covalent bonds that form under physiological conditions, wherein L₃ and L₁ are selected from the group consisting of linker element pairs (1) to (14).

78. The therapeutic composition of claim 1 further comprising:
a third precursor compound having the chemical structure:
E3ULB₂—C₃-L₃,
or a pharmaceutically acceptable salt, enantiomer, stereoisomer, solvate, or polymorph thereof, and
a fourth precursor compound having the chemical structure:
TPB₂—C₄-L₄,
or a pharmaceutically acceptable salt, enantiomer, stereoisomer, solvate, or polymorph thereof, wherein:
E3ULB₂ is a small molecule E3 ubiquitin ligase binding moiety that binds an E3 ubiquitin ligase, an E3 ubiquitin ligase complex, or subunit thereof that differs in structure from E3ULB,
TPB₂ is a small molecule comprising a BET domain protein binding moiety that differs in structure from TPB,
C₃ and C₄ are bonds or connector elements, and
L₃ and L₄ are linker elements having a molecular weight of 54 to 420 Daltons, with L₁ being capable of binding to L₂ or L₄ but not to L₃, with L₃ being capable of binding to L₂ or L₄ but not L₁, by two or more reversible covalent bonds that form under physiological conditions, wherein L₃ and L₄ are selected from the group consisting of linker element pairs (1) to (13).

79. A method of binding to and redirecting the specificity of an E3 ubiquitin ligase, an E3 ubiquitin ligase complex, or subunit thereof to induce the ubiquitination and degradation of a BET domain protein in a biological sample, comprising:
contacting the sample with the composition of claim 1.

80. A method of treating a BET domain protein mediated disorder, condition, or disease in a patient comprising:
administering to said patient the composition of claim 1.

81.-82. (canceled)


SEQUENCE LISTING (14 sequences)

SEQ ID NO: 1 - protein, 14 aa, Artificial Sequence
  V5 tag
GKPIPNPLLGLDST

SEQ ID NO: 2 - protein, 9 aa, Artificial Sequence
  V5 tag
IPNPLLGLD

SEQ ID NO: 3 - protein, 8 aa, Artificial Sequence
  FLAG-tag
DYKDDDDK

SEQ ID NO: 4 - protein, 10 aa, Artificial Sequence
  Myc-tag
EQKLISEEDL

SEQ ID NO: 5 - protein, 9 aa, Artificial Sequence
  HA-tag
YPYDVPDYA

SEQ ID NO: 6 - protein, 6 aa, Artificial Sequence
  His-tag
HHHHHH

SEQ ID NO: 7 - protein, 11 aa, Artificial Sequence
  LgBiT to capture HiBiT 11-amino acid tag
VSGWRLFKKIS

SEQ ID NO: 8 - protein, 12 aa, Artificial Sequence
  tetracysteine binding mofif
FLNCCPGCCMEP

SEQ ID NO: 9 - DNA, 19 bp, Artificial Sequence
  GAPDH-F primer
agccacatcgctcagacac

SEQ ID NO: 10 - DNA, 19 bp, Artificial Sequence
  GAPDH-R primer
gcccaatacgaccaaatcc

SEQ ID NO: 11 - DNA, 18 bp, Artificial Sequence
  ACTB-F primer
ccaaccgcgagaagatga

SEQ ID NO: 12 - DNA, 20 bp, Artificial Sequence
  ACTB-R primer
ccagaggcgtacagggatag

SEQ ID NO: 13 - DNA, 20 bp, Artificial Sequence
  SLC19A1-F primer
atggcccccaagaagtagat

SEQ ID NO: 14 - DNA, 20 bp, Artificial Sequence
  SLC19A1-R primer
gtcaacacgttctttgccac